(19) United States
(12) Patent Application Publication
(10) Pub. No.: US 2026/0139051 A1
(43) Pub. Date: May 21, 2026

(54) COMBINATION THERAPIES USING MOLECULES BINDING TO TCR

(71) Applicant: Marengo Therapeutics, Inc., Cambridge, MA (US)

(72) Inventors: Madan Katragadda, Acton, MA (US); Andrew Bayliffe, London (GB)

(21) Appl. No.: 19/370,251

(22) Filed: Oct. 27, 2025

Related U.S. Application Data
(63) Continuation of application No. PCT/US24/26686, filed on Apr. 26, 2024.
(60) Provisional application No. 63/498,671, filed on Apr. 27, 2023.

(51) Int. Cl. C07K 16/28; A61K 38/00; A61K 39/00; A61P 35/00; C07K 14/54; C07K 14/55; C07K 14/715; C07K 16/32

(52) U.S. Cl. CPC C07K 16/2809; A61P 35/00; C07K 14/5434; C07K 14/5443; C07K 14/55; C07K 14/7155; C07K 16/2803; C07K 16/2866; C07K 16/32; A61K 38/00; A61K 2039/505; C07K 2317/31; C07K 2317/52; C07K 2317/565; C07K 2319/70

(57) ABSTRACT

Provides herein is method of treating a disease or condition in a subject, or a combination therapy using agents that bind to a T cell receptor alpha variable region (TCRαV) and/or a T cell receptor beta variable region (TCRβV).

### RELATED APPLICATIONS

This application is a continuation of International Application No. PCT/US2024/026686 filed on Apr. 26, 2024, which claims the benefit of U.S. Provisional Application No. 63/498,671 filed on Apr. 27, 2023, each of which is incorporated herein by reference in its entirety

### SEQUENCE LISTING

The instant application contains a Sequence listing which has been submitted electronically in XML format and is herein incorporated by reference in its entirety. Said XML copy, created on Jun. 18, 2024, is named 53676-758_301_SL.xml and is 1,397,765 bytes in size.

### BACKGROUND

Currently available molecules designed to redirect T cells to promote tumor cell lysis for cancer immunotherapy typically target the CD3 epsilon (CD3e) subunit of the T cell receptor (TCR). However, there are limitations to this approach. Previous studies have shown that, e.g., low doses of anti-CD3e monoclonal antibody (mAb) can cause T cell dysfunction and exert immunosuppressive effects. In addition, anti-CD3e mAbs bind to all T cells and thus activate a large number of T cells. Such non-physiological massive activation of T cells by these anti-CD3e mAbs can result in the production of proinflammatory cytokines such as IFN-gamma, IL-1-beta, IL-6, IL-10 and TNF-alpha, causing a “cytokine storm” known as the cytokine release syndrome (CRS), which is also associated with neurotoxicity (NT). Thus, there is a need for improved T cell receptor-binding molecules that redirect T cells for cancer immunotherapy.

### SUMMARY

In an aspect, provided herein is, inter alia, a method of treating a disease or condition in a subject in need thereof comprising administering a first agent that comprises a first binder that binds to a first target molecule to the subject, and administering a second agent that comprises a second binder that binds to a second target molecule to the subject, thereby treating the disease or condition in the subject; wherein an administration of the first agent is followed by an administration of the second agent; wherein the first target molecule and the second target molecule are independently selected from the group consisting of a T cell receptor alpha variable region (TCRαV) and a T cell receptor beta variable region (TCRβV); and wherein the first target molecule and the second target molecule are different.

In some embodiments, the first agent and the second agent are not concomitantly administered to the subject.

In some embodiments, administering the first agent comprises administering a therapeutically effective amount of the first agent to the subject.

In some embodiments, administering the second agent comprises administering a therapeutically effective amount of the second agent to the subject.

In some embodiments, the method is more effective to treat the disease or condition in the subject relative to a method in which the first agent is administered to the subject but not the second agent.

In some embodiments, the method is more effective to treat the disease or condition in the subject relative to a method in which the second agent is administered to the subject but not the first agent.

In some embodiments, the method is more effective to treat the disease or condition in the subject relative to a method in which the first agent is administered to the subject at a first administration and the first agent is administered to the subject subsequently at a second administration.

In some embodiments, the method is more effective to treat the disease or condition in the subject relative to a method in which the second agent is administered to the subject at a first administration and the second agent is administered to the subject subsequently at a second administration.

In some embodiments, the method is more effective to treat the disease or condition in the subject relative to a method in which the second agent is administered to the subject at a first administration and the first agent is administered to the subject subsequently at a second administration.

In some embodiments, the administration of the first agent comprises one or more doses.

In some embodiments, the administration of the first agent comprises two or more doses.

In some embodiments, the administration of the first agent comprises one dose per week.

In some embodiments, the administration of the first agent comprises two or more doses per week.

In some embodiments, the first agent is administered to the subject once every two weeks.

In some embodiments, the first agent is administered to the subject once every three weeks.

In some embodiments, the first agent is administered to the subject once every week for at least 1, 2, 3, or 4 weeks, at least 1, 2, 3, 4, 5, 6, 7, 8, 9, 10, 11 or 12 months, or at least 1, 2, or 3 years.

In some embodiments, the administration of the first agent comprises two or more doses per week for at least 1, 2, 3, or 4 weeks, at least 1, 2, 3, 4, 5, 6, 7, 8, 9, 10, 11 or 12 months, or at least 1, 2, or 3 years.

In some embodiments, the first agent is administered to the subject once every two weeks for at least 1, 2, 3, or 4 weeks, at least 1, 2, 3, 4, 5, 6, 7, 8, 9, 10, 11 or 12 months, or at least 1, 2, or 3 years.

In some embodiments, the first agent is administered to the subject once every three weeks for at least 1, 2, 3, or 4 weeks, at least 1, 2, 3, 4, 5, 6, 7, 8, 9, 10, 11 or 12 months, or at least 1, 2, or 3 years.

In some embodiments, the administration of the second agent comprises one or more doses.

In some embodiments, the administration of the second agent comprises two or more doses.

In some embodiments, the administration of the second agent comprises one dose per week.

In some embodiments, the administration of the second agent comprises two or more doses per week.

In some embodiments, the second agent is administered to the subject once every two weeks.

In some embodiments, the second agent is administered to the subject once every three weeks.

In some embodiments, the second agent is administered to the subject once every week for at least 1, 2, 3, or 4 weeks, at least 1, 2, 3, 4, 5, 6, 7, 8, 9, 10, 11 or 12 months, or at least 1, 2, or 3 years.

In some embodiments, the administration of the second agent comprises two or more doses per week for at least 1, 2, 3, or 4 weeks, at least 1, 2, 3, 4, 5, 6, 7, 8, 9, 10, 11 or 12 months, or at least 1, 2, or 3 years.

In some embodiments, the second agent is administered to the subject once every two weeks for at least 1, 2, 3, or 4 weeks, at least 1, 2, 3, 4, 5, 6, 7, 8, 9, 10, 11 or 12 months, or at least 1, 2, or 3 years.

In some embodiments, the second agent is administered to the subject once every three weeks for at least 1, 2, 3, or 4 weeks, at least 1, 2, 3, 4, 5, 6, 7, 8, 9, 10, 11 or 12 months, or at least 1, 2, or 3 years.

In some embodiments, the administration of the first agent and the administration of the second agent is separated by one week.

In some embodiments, the administration of the first agent and the administration of the second agent is separated by two or more weeks.

In some embodiments, the administration of the first agent and the administration of the second agent is separated by 1 day, 2 days, 3 days, 4 days, 5 days, 6 days, 1 week, 2 weeks, 3 weeks, 4 weeks, 1 month, 2 months, 3 months, 4 months, 5 months, 6 months, 7 months, 8 months, 9 months, 10 months, 11 months, or 12 months.

In some embodiments, the administration of the first agent and the second agent is repeated twice or more.

In some embodiments, the first agent and the second agent are administered to the subject according to a dosing regimen, wherein the dosing regimen comprises an administration of the first agent once every week for two weeks followed by an administration of the second agent once a week for two weeks, wherein the second agent is administered to the subject one week after the last administration of the first agent.

In some embodiments, the dosing regimen is repeated twice or more.

In some embodiments, the dosing regimen is repeated at the interval of one week.

In some embodiments, the dosing regimen is repeated at the interval of 1 day, 2 days, 3 days, 4 days, 5 days, 6 days, 1 week, 2 weeks, 3 weeks, 4 weeks, 1 month, 2 months, 3 months, 4 months, 5 months, 6 months, 7 months, 8 months, 9 months, 10 months, 11 months, or 12 months.

In some embodiments, the first agent and the second agent are administered to the subject in a same amount.

In some embodiments, the first agent and the second agent are administered to the subject in different amounts.

In some embodiments, the administration of the first agent comprises two or more doses, and each dose comprises a same amount of the first agent.

In some embodiments, the administration of the first agent comprises two or more doses, and each dose comprises different amounts of the first agent.

In some embodiments, the administration of the second agent comprises two or more doses, and each dose comprises a same amount of the second agent.

In some embodiments, the administration of the second agent comprises two or more doses, and each dose comprises different amounts of the second agent.

In some embodiments, the first agent is administered to the subject at a dose of from about 0.001 mg/kg to about 10 mg/kg.

In some embodiments, the second agent is administered to the subject at a dose of from about 0.001 mg/kg to about 10 mg/kg.

In some embodiments, the first agent is administered to the subject at a dose of about 0.3 mg/kg or about 1 mg/kg.

In some embodiments, the second agent is administered to the subject at a dose of about 0.3 mg/kg or about 1 mg/kg.

In some embodiments, the first target molecule is a first TCRβV, and the second target molecule is a second TCRβV, wherein the first TCRβV and the second TCRβV are different.

In some embodiments, the first target molecule is a first TCRαV, and the second target molecule is a second TCRαV, wherein the first TCRαV and the second TCRαV are different.

In some embodiments, the first target molecule is a TCRβV, and the second target molecule is a TCRαV.

In some embodiments, the first target molecule is a TCRαV, and the second target molecule is a TCRβV.

In some embodiments, the first target molecule is human TCRβV6.

In some embodiments, the first target molecule is human TCRβV10.

In some embodiments, the second target molecule is human TCRβV15.

In some embodiments, the second target molecule is human TCRβV27.

In some embodiments, the second target molecule is human TCRβV5.

In some embodiments, the first target molecule is human TCRβV6, and the second target molecule is human TCRβV5.

In some embodiments, the first target molecule is human TCRβV6, and the second target molecule is human TCRβV15.

In some embodiments, the first target molecule is human TCRβV6, and the second target molecule is human TCRβV27.

In some embodiments, the first target molecule is human TCRβV10, and the second target molecule is human TCRβV5.

In some embodiments, the first target molecule is human TCRβV10, and the second target molecule is human TCRβV15.

In some embodiments, the first target molecule is human TCRβV10, and the second target molecule is human TCRβV27.

In some embodiments, the first target molecule is human TRBV5, human TRBV6, human TRBV10, human TRBV7, human TRBV19, human TRBV20, or human TRBV28.

In some embodiments, the first target molecule is human TRBV5-1, human TRBV6-5, human TRBV10-1, human TRBV10-2, human TRBV10-3, human TRBV7-2, human TRBV7-3, human TRBV19, human TRBV20-1, or human TRBV28.

In some embodiments, the first target molecule is human TRAV12, human TRAV13, human TRAV19, human TRAV21, or human TRAV30.

In some embodiments, the first target molecule is human TRAV12-2, human TRAV13-1, human TRAV19, human TRAV21, or human TRAV30.

In some embodiments, the second target molecule is human TRBV5, human TRBV6, human TRBV10, human TRBV7, human TRBV15, human TRBV27, human TRBV19, human TRBV20, or human TRBV28.

In some embodiments, the second target molecule is human TRBV5-1, human TRBV6-5, human TRBV10-1, human TRBV10-2, human TRBV10-3, human TRBV7-2, human TRBV7-3, human TRBV15, human TRBV27, human TRBV19, human TRBV20-1, or human TRBV28.

In some embodiments, the second target molecule is human TRAV12, human TRAV13, human TRAV19, human TRAV21, or human TRAV30.

In some embodiments, the second target molecule is human TRAV12-2, human TRAV13-1, human TRAV19, human TRAV21, or human TRAV30.

In some embodiments, the first target molecule is human TRBV5, human TRBV6, human TRBV10, human TRBV7, human TRBV19, human TRBV20, or human TRBV28, and the second target molecule is human TRBV5, human TRBV6, human TRBV10, human TRBV7, human TRBV15, human TRBV27, human TRBV19, human TRBV20, or human TRBV28, wherein the first target molecule and the second target molecule are different.

In some embodiments, the first target molecule and the second target molecule are: human TRBV5 and human TRBV5, respectively; human TRBV5 and human TRBV6, respectively; human TRBV5 and human TRBV10, respectively; human TRBV5 and human TRBV7, respectively; human TRBV5 and human TRBV19, respectively; human TRBV5 and human TRBV20, respectively; human TRBV5 and human TRBV28, respectively; human TRBV6 and human TRBV5, respectively; human TRBV6 and human TRBV6, respectively; human TRBV6 and human TRBV10, respectively; human TRBV6 and human TRBV7, respectively; human TRBV6 and human TRBV15, respectively; human TRBV6 and human TRBV27, respectively; human TRBV6 and human TRBV19, respectively; human TRBV6 and human TRBV20, respectively; human TRBV6 and human TRBV28, respectively; human TRBV10 and human TRBV5, respectively; human TRBV10 and human TRBV10, respectively; human TRBV10 and human TRBV6, respectively; human TRBV10 and human TRBV7, respectively; human TRBV10 and human TRBV15, respectively; human TRBV10 and human TRBV27, respectively; human TRBV10 and human TRBV19, respectively; human TRBV10 and human TRBV20, respectively; human TRBV10 and human TRBV28, respectively; human TRBV7 and human TRBV5, respectively; human TRBV7 and human TRBV6, respectively; human TRBV7 and human TRBV10, respectively; human TRBV7 and human TRBV7, respectively; human TRBV7 and human TRBV19, respectively; human TRBV7 and human TRBV20, respectively; human TRBV7 and human TRBV28, respectively; human TRBV19 and human TRBV5, respectively; human TRBV19 and human TRBV6, respectively; human TRBV19 and human TRBV10, respectively; human TRBV19 and human TRBV7, respectively, human TRBV19 and human TRBV19, respectively; human TRBV19 and human TRBV20, respectively; human TRBV19 and human TRBV28, respectively; human TRBV20 and human TRBV5, respectively; human TRBV20 and human TRBV6, respectively; human TRBV20 and human TRBV10, respectively; human TRBV20 and human TRBV7, respectively; human TRBV20 and human TRBV19, respectively; human TRBV20 and human TRBV20, respectively; human TRBV20 and human TRBV28, respectively; human TRBV28 and human TRBV5, respectively; human TRBV28 and human TRBV6, respectively; human TRBV28 and human TRBV10, respectively; human TRBV28 and human TRBV7, respectively, human TRBV28 and human TRBV19, respectively; human TRBV28 and human TRBV20, respectively; or human TRBV28 and human TRBV28, respectively.

In some embodiments, the first target molecule is human TRBV5-1, human TRBV6-5, human TRBV10-1, human TRBV10-2, human TRBV10-3, human TRBV7-2, human TRBV7-3, human TRBV19, human TRBV20-1, or human TRBV28, and the second target molecule is human TRBV5-1, human TRBV6-5, human TRBV10-1, human TRBV10-2, human TRBV10-3, human TRBV7-2, human TRBV7-3, human TRBV15, human TRBV27, human TRBV19, human TRBV20-1, or human TRBV28, wherein the first target molecule and the second target molecule are different.

In some embodiments, the first target molecule and the second target molecule are: human TRBV5-1 and human TRBV6-5, respectively; human TRBV5-1 and human TRBV10-1, respectively; human TRBV5-1 and human TRBV10-2, respectively; human TRBV5-1 and human TRBV10-3, respectively; human TRBV5-1 and human TRBV7-2, respectively; human TRBV5-1 and human TRBV7-3, respectively; human TRBV5-1 and human TRBV19, respectively; human TRBV5-1 and human TRBV20-1, respectively; human TRBV5-1 and human TRBV28, respectively; human TRBV6-5 and human TRBV5-1, respectively; human TRBV6-5 and human TRBV7-2, respectively; human TRBV6-5 and human TRBV7-3, respectively; human TRBV6-5 and human TRBV15, respectively; human TRBV6-5 and human TRBV27, respectively; human TRBV6-5 and human TRBV19, respectively; human TRBV6-5 and human TRBV20-1, respectively; human TRBV6-5 and human TRBV28, respectively; human TRBV10-1 and human TRBV5-1, respectively; human TRBV10-1 and human TRBV7-2, respectively; human TRBV10-1 and human TRBV7-3, respectively; human TRBV10-1 and human TRBV15, respectively; human TRBV10-1 and human TRBV27, respectively; human TRBV10-1 and human TRBV19, respectively; human TRBV10-1 and human TRBV20-1, respectively; human TRBV10-1 and human TRBV28, respectively; human TRBV10-2 and human TRBV5-1, respectively; human TRBV10-2 and human TRBV7-2, respectively; human TRBV10-2 and human TRBV7-3, respectively; human TRBV10-2 and human TRBV15, respectively; human TRBV10-2 and human TRBV27, respectively; human TRBV10-2 and human TRBV19, respectively; human TRBV10-2 and human TRBV20-1, respectively; human TRBV10-2 and human TRBV28, respectively; human TRBV10-3 and human TRBV5-1, respectively; human TRBV10-3 and human TRBV7-2, respectively; human TRBV10-3 and human TRBV7-3, respectively; human TRBV10-3 and human TRBV15, respectively; human TRBV10-3 and human TRBV27, respectively; human TRBV10-3 and human TRBV19, respectively; human TRBV10-3 and human TRBV20-1, respectively; human TRBV10-3 and human TRBV28, respectively; human TRBV7-2 and human TRBV5-1, respectively; human TRBV7-2 and human TRBV6-5, respectively; human TRBV7-2 and human TRBV10-1, respectively; human TRBV7-2 and human TRBV10-2, respectively; human TRBV7-2 and human TRBV10-3, respectively; human TRBV7-2 and human TRBV7-3, respectively; human TRBV7-2 and human TRBV19, respectively; human TRBV7-2 and human TRBV20-1, respectively; human TRBV7-2 and human TRBV28, respectively; human TRBV7-3 and human TRBV5-1, respectively; human TRBV7-3 and human TRBV6-5, respectively; human TRBV7-3 and human TRBV10-1, respectively; human TRBV7-3 and human TRBV10-2, respectively; human TRBV7-3 and human TRBV10-3, respectively; human TRBV7-3 and human TRBV7-2, respectively; human TRBV7-3 and human TRBV19, respectively; human TRBV7-3 and human TRBV20-1, respectively; human TRBV7-3 and human TRBV28, respectively; human TRBV19 and human TRBV5-1, respectively; human TRBV19 and human TRBV6-5, respectively; human TRBV19 and human TRBV10-1, respectively; human TRBV19 and human TRBV10-2, respectively; human TRBV19 and human TRBV10-3, respectively; human TRBV19 and human TRBV7-2, respectively; human TRBV19 and human TRBV7-3, respectively; human TRBV19 and human TRBV20-1, respectively; human TRBV19 and human TRBV28, respectively; human TRBV20-1 and human TRBV5-1, respectively; human TRBV20-1 and human TRBV6-5, respectively; human TRBV20-1 and human TRBV10-1, respectively; human TRBV20-1 and human TRBV10-2, respectively; human TRBV20-1 and human TRBV10-3, respectively; human TRBV20-1 and human TRBV7-2, respectively; human TRBV20-1 and human TRBV7-3, respectively;
human TRBV20-1 and human TRBV19, respectively; human TRBV20-1 and human TRBV28, respectively; human TRBV28 and human TRBV5-1, respectively; human TRBV28 and human TRBV6-5, respectively; human TRBV28 and human TRBV10-1, respectively; human TRBV28 and human TRBV10-2, respectively; human TRBV28 and human TRBV10-3, respectively; human TRBV28 and human TRBV7-2, respectively; human TRBV28 and human TRBV7-3, respectively; human TRBV28 and human TRBV19, respectively; or human TRBV28 and human TRBV20-1, respectively.

In some embodiments, the first target molecule is human TRAV12, human TRAV13, human TRAV19, human TRAV21, or human TRAV30, and the second target molecule is human TRAV12, human TRAV13, human TRAV19, human TRAV21, or human TRAV30, wherein the first target molecule and the second target molecule are different.

In some embodiments, the first target molecule and the second target molecule are:
human TRAV12 and human TRAV12, respectively;
human TRAV12 and human TRAV13, respectively;
human TRAV12 and human TRAV19, respectively;
human TRAV12 and human TRAV21, respectively;
human TRAV12 and human TRAV30, respectively;
human TRAV13 and human TRAV12, respectively;
human TRAV13 and human TRAV13, respectively;
human TRAV13 and human TRAV19, respectively;
human TRAV13 and human TRAV21, respectively;
human TRAV13 and human TRAV30, respectively;
human TRAV19 and human TRAV12, respectively;
human TRAV19 and human TRAV13, respectively;
human TRAV19 and human TRAV19, respectively;
human TRAV19 and human TRAV21, respectively;
human TRAV19 and human TRAV30, respectively;
human TRAV21 and human TRAV12, respectively;
human TRAV21 and human TRAV13, respectively;
human TRAV21 and human TRAV19, respectively;
human TRAV21 and human TRAV21, respectively;
human TRAV21 and human TRAV30, respectively;
human TRAV30 and human TRAV12, respectively;
human TRAV30 and human TRAV13, respectively;
human TRAV30 and human TRAV19, respectively;
human TRAV30 and human TRAV21, respectively; or
human TRAV30 and human TRAV30, respectively.

In some embodiments, the first target molecule is human TRAV12-2, human TRAV13-1, human TRAV19, human TRAV21, or human TRAV30, and the second target molecule is human TRAV12-2, human TRAV13-1, human TRAV19, human TRAV21, or human TRAV30, wherein the first target molecule and the second target molecule are different.

In some embodiments, the first target molecule and the second target molecule are:
human TRAV12-2 and human TRAV13-1, respectively;
human TRAV12-2 and human TRAV19, respectively;
human TRAV12-2 and human TRAV21, respectively;
human TRAV12-2 and human TRAV30, respectively;
human TRAV13-1 and human TRAV12-2, respectively;
human TRAV13-1 and human TRAV19, respectively;
human TRAV13-1 and human TRAV21, respectively;
human TRAV13-1 and human TRAV30, respectively;
human TRAV19 and human TRAV12-2, respectively;
human TRAV19 and human TRAV13-1, respectively;
human TRAV19 and human TRAV21, respectively;
human TRAV19 and human TRAV30, respectively;
human TRAV21 and human TRAV12-2, respectively;
human TRAV21 and human TRAV13-1, respectively;
human TRAV21 and human TRAV19, respectively;
human TRAV21 and human TRAV30, respectively;
human TRAV30 and human TRAV12-2, respectively;
human TRAV30 and human TRAV13-1, respectively;
human TRAV30 and human TRAV19, respectively; or
human TRAV30 and human TRAV21, respectively.

In some embodiments, the first target molecule is human TRBV5, human TRBV6, human TRBV10, human TRBV7, human TRBV19, human TRBV20, or human TRBV28, and the second target molecule is human TRAV12, human TRAV13, human TRAV19, human TRAV21, or human TRAV30.

In some embodiments, the first target molecule and the second target molecule are:
human TRBV5 and human TRAV12, respectively; human TRBV5 and human TRAV13, respectively;
human TRBV5 and human TRAV19, respectively; human TRBV5 and human TRAV21, respectively;
human TRBV5 and human TRAV30, respectively; human TRBV6 and human TRAV12, respectively;
human TRBV6 and human TRAV13, respectively; human TRBV6 and human TRAV19, respectively;
human TRBV6 and human TRAV21, respectively; human TRBV6 and human TRAV30, respectively;
human TRBV10 and human TRAV12, respectively; human TRBV10 and human TRAV13, respectively;
human TRBV10 and human TRAV19, respectively; human TRBV10 and human TRAV21, respectively;
human TRBV10 and human TRAV30, respectively; human TRBV7 and human TRAV12, respectively;
human TRBV7 and human TRAV13, respectively; human TRBV7 and human TRAV19, respectively;
human TRBV7 and human TRAV21, respectively; human TRBV7 and human TRAV30, respectively;
human TRBV19 and human TRAV12, respectively; human TRBV19 and human TRAV13, respectively;
human TRBV19 and human TRAV19, respectively; human TRBV19 and human TRAV21, respectively;
human TRBV19 and human TRAV30, respectively; human TRBV20 and human TRAV12, respectively;
human TRBV20 and human TRAV13, respectively; human TRBV20 and human TRAV19, respectively;
human TRBV20 and human TRAV21, respectively; human TRBV20 and human TRAV30, respectively;
human TRBV28 and human TRAV12, respectively; human TRBV28 and human TRAV13, respectively;
human TRBV28 and human TRAV19, respectively; human TRBV28 and human TRAV21, respectively;
or human TRBV28 and human TRAV30, respectively.

In some embodiments, the first target molecule is human TRBV5, human TRBV6, human TRBV10, human TRBV7, human TRBV19, human TRBV20, or human TRBV28, and the second target molecule is human TRAV12-2, human TRAV13-1, human TRAV19, human TRAV21, or human TRAV30.

In some embodiments, the first target molecule and the second target molecule are: human TRBV5 and human TRAV12-2, respectively; human TRBV5 and human TRAV13-1, respectively; human TRBV5 and human TRAV19, respectively; human TRBV5 and human TRAV21, respectively; human TRBV5 and human TRAV30, respectively; human TRBV6 and human TRAV12-2, respectively; human TRBV6 and human TRAV13-1, respectively; human TRBV6 and human TRAV19, respectively; human TRBV6 and human TRAV21, respectively; human TRBV6 and human TRAV30, respectively; human TRBV10 and human TRAV12-2, respectively; human TRBV10 and human TRAV13-1, respectively; human TRBV10 and human TRAV19, respectively; human TRBV10 and human TRAV21, respectively; human TRBV10 and human TRAV30, respectively; human TRBV7 and human TRAV12-2, respectively; human TRBV7 and human TRAV13-1, respectively; human TRBV7 and human TRAV19, respectively; human TRBV7 and human TRAV21, respectively; human TRBV7 and human TRAV30, respectively; human TRBV19 and human TRAV12-2, respectively; human TRBV19 and human TRAV13-1, respectively; human TRBV19 and human TRAV19, respectively; human TRBV19 and human TRAV21, respectively; human TRBV19 and human TRAV30, respectively; human TRBV20 and human TRAV12-2, respectively; human TRBV20 and human TRAV13-1, respectively; human TRBV20 and human TRAV19, respectively; human TRBV20 and human TRAV21, respectively; human TRBV20 and human TRAV30, respectively; human TRBV28 and human TRAV12-2, respectively; human TRBV28 and human TRAV13-1, respectively; human TRBV28 and human TRAV19, respectively; human TRBV28 and human TRAV21, respectively; or human TRBV28 and human TRAV30, respectively.

In some embodiments, the first target molecule is human TRBV5-1, human TRBV6-5, human TRBV10-1, human TRBV10-2, human TRBV10-3, human TRBV7-2, human TRBV7-3, human TRBV19, human TRBV20-1, or human TRBV28, and the second target molecule is human TRAV12, human TRAV13, human TRAV19, human TRAV21, or human TRAV30.

In some embodiments, the first target molecule and the second target molecule are: human TRBV5-1 and human TRAV12, respectively; human TRBV5-1 and human TRAV13, respectively; human TRBV5-1 and human TRAV19, respectively; human TRBV5-1 and human TRAV21, respectively; human TRBV5-1 and human TRAV30, respectively; human TRBV6-5 and human TRAV12, respectively; human TRBV6-5 and human TRAV13, respectively; human TRBV6-5 and human TRAV19, respectively; human TRBV6-5 and human TRAV21, respectively; human TRBV6-5 and human TRAV30, respectively; human TRBV10-1 and human TRAV12, respectively; human TRBV10-1 and human TRAV13, respectively; human TRBV10-1 and human TRAV19, respectively; human TRBV10-1 and human TRAV21, respectively; human TRBV10-1 and human TRAV30, respectively; human TRBV10-2 and human TRAV12, respectively; human TRBV10-2 and human TRAV13, respectively; human TRBV10-2 and human TRAV19, respectively; human TRBV10-2 and human TRAV21, respectively; human TRBV10-2 and human TRAV30, respectively; human TRBV10-3 and human TRAV12, respectively; human TRBV10-3 and human TRAV13, respectively; human TRBV10-3 and human TRAV19, respectively; human TRBV10-3 and human TRAV21, respectively; human TRBV10-3 and human TRAV30, respectively; human TRBV7-2 and human TRAV12, respectively; human TRBV7-2 and human TRAV13, respectively; human TRBV7-2 and human TRAV19, respectively; human TRBV7-2 and human TRAV21, respectively; human TRBV7-2 and human TRAV30, respectively; human TRBV7-3 and human TRAV12, respectively; human TRBV7-3 and human TRAV13, respectively; human TRBV7-3 and human TRAV19, respectively; human TRBV7-3 and human TRAV21, respectively; human TRBV7-3 and human TRAV30, respectively; human TRBV19 and human TRAV12, respectively, human TRBV19 and human TRAV13, respectively; human TRBV19 and human TRAV19, respectively; human TRBV19 and human TRAV21, respectively; human TRBV19 and human TRAV30, respectively; human TRBV20-1 and human TRAV12, respectively; human TRBV20-1 and human TRAV13, respectively; human TRBV20-1 and human TRAV19, respectively; human TRBV20-1 and human TRAV21, respectively; human TRBV20-1 and human TRAV30, respectively; human TRBV28 and human TRAV12, respectively; human TRBV28 and human TRAV13, respectively; human TRBV28 and human TRAV19, respectively; human TRBV28 and human TRAV21, respectively; or human TRBV28 and human TRAV30, respectively.

In some embodiments, the first target molecule is human TRBV5-1, human TRBV6-5, human TRBV10-1, human TRBV10-2, human TRBV10-3, human TRBV7-2, human TRBV7-3, human TRBV19, human TRBV20-1, or human TRBV28, and the second target molecule is human TRAV12-2, human TRAV13-1, human TRAV19, human TRAV21, or human TRAV30.

In some embodiments, the first target molecule and the second target molecule are:
human TRBV5-1 and human TRAV12-2, respectively; human TRBV5-1 and human TRAV13-1, respectively; human TRBV5-1 and human TRAV19, respectively; human TRBV5-1 and human TRAV21, respectively; human TRBV5-1 and human TRAV30, respectively; human TRBV6-5 and human TRAV12-2, respectively; human TRBV6-5 and human TRAV13-1, respectively; human TRBV6-5 and human TRAV19, respectively; human TRBV6-5 and human TRAV21, respectively; human TRBV6-5 and human TRAV30, respectively; human TRBV10-1 and human TRAV12-2, respectively; human TRBV10-1 and human TRAV13-1, respectively; human TRBV10-1 and human TRAV19, respectively; human TRBV10-1 and human TRAV21, respectively; human TRBV10-1 and human TRAV30, respectively; human TRBV10-2 and human TRAV12-2, respectively; human TRBV10-2 and human TRAV13-1, respectively; human TRBV10-2 and human TRAV19, respectively; human TRBV10-2 and human TRAV21, respectively; human TRBV10-2 and human TRAV30, respectively; human TRBV10-3 and human TRAV12-2, respectively; human TRBV10-3 and human TRAV13-1, respectively; human TRBV10-3 and human TRAV19, respectively; human TRBV10-3 and human TRAV21, respectively; human TRBV10-3 and human TRAV30, respectively; human TRBV7-2 and human TRAV12-2, respectively; human TRBV7-2 and human TRAV13-1, respectively; human TRBV7-2 and human TRAV19, respectively; human TRBV7-2 and human TRAV21, respectively; human TRBV7-2 and human TRAV30, respectively, human TRBV7-3 and human TRAV12-2, respectively; human TRBV7-3 and human TRAV13-1, respectively; human TRBV7-3 and human TRAV19, respectively; human TRBV7-3 and human TRAV21, respectively; human TRBV7-3 and human TRAV30, respectively; human TRBV19 and human TRAV12-2, respectively; human TRBV19 and human TRAV13-1, respectively; human TRBV19 and human TRAV19, respectively; human TRBV19 and human TRAV21, respectively; human TRBV19 and human TRAV30, respectively; human TRBV20-1 and human TRAV12-2, respectively; human TRBV20-1 and human TRAV13-1, respectively; human TRBV20-1 and human TRAV19, respectively; human TRBV20-1 and human TRAV21, respectively; human TRBV20-1 and human TRAV30, respectively; human TRBV28 and human TRAV12-2, respectively; human TRBV28 and human TRAV13-1, respectively; human TRBV28 and human TRAV19, respectively; human TRBV28 and human TRAV21, respectively; or human TRBV28 and human TRAV30, respectively.

In some embodiments, the first target molecule is human TRAV12, human TRAV13, human TRAV19, human TRAV21, or human TRAV30, and the second target molecule is human TRBV5, human TRBV6, human TRBV10, human TRBV7, human TRBV19, human TRBV20, or human TRBV28.

In some embodiments, the first target molecule and the second target molecule are:
human TRAV12 and human TRBV5, respectively; human TRAV12 and human TRBV6, respectively; human TRAV12 and human TRBV10, respectively; human TRAV12 and human TRBV7, respectively; human TRAV12 and human TRBV19, respectively; human TRAV12 and human TRBV20, respectively; human TRAV12 and human TRBV28, respectively; human TRAV13 and human TRBV5, respectively; human TRAV13 and human TRBV6, respectively; human TRAV13 and human TRBV10, respectively; human TRAV13 and human TRBV7, respectively; human TRAV13 and human TRBV19, respectively; human TRAV13 and human TRBV20, respectively; human TRAV13 and human TRBV28, respectively; human TRAV19 and human TRBV5, respectively; human TRAV19 and human TRBV6, respectively; human TRAV19 and human TRBV10, respectively; human TRAV19 and human TRBV7, respectively; human TRAV19 and human TRBV19, respectively; human TRAV19 and human TRBV20, respectively; human TRAV19 and human TRBV28, respectively; human TRAV21 and human TRBV5, respectively; human TRAV21 and human TRBV6, respectively; human TRAV21 and human TRBV10, respectively; human TRAV21 and human TRBV7, respectively; human TRAV21 and human TRBV19, respectively; human TRAV21 and human TRBV20, respectively; human TRAV21 and human TRBV28, respectively; human TRAV30 and human TRBV5, respectively; human TRAV30 and human TRBV6, respectively; human TRAV30 and human TRBV10, respectively; human TRAV30 and human TRBV7, respectively; human TRAV30 and human TRBV19, respectively; human TRAV30 and human TRBV20, respectively; or human TRAV30 and human TRBV28, respectively.

In some embodiments, the first target molecule is human TRAV12-2, human TRAV13-1, human TRAV19, human TRAV21, or human TRAV30, and the second target molecule is human TRBV5, human TRBV6, human TRBV10, human TRBV7, human TRBV19, human TRBV20, or human TRBV28.

In some embodiments, the first target molecule and the second target molecule are: human TRAV12-2 and human TRBV5, respectively; human TRAV12-2 and human TRBV6, respectively; human TRAV12-2 and human TRBV10, respectively; human TRAV12-2 and human TRBV7, respectively; human TRAV12-2 and human TRBV19, respectively; human TRAV12-2 and human TRBV20, respectively; human TRAV12-2 and human TRBV28, respectively; human TRAV13-1 and human TRBV5, respectively; human TRAV13-1 and human TRBV6, respectively; human TRAV13-1 and human TRBV10, respectively; human TRAV13-1 and human TRBV7, respectively; human TRAV13-1 and human TRBV19, respectively; human TRAV13-1 and human TRBV20, respectively; human TRAV13-1 and human TRBV28, respectively; human TRAV19 and human TRBV5, respectively; human TRAV19 and human TRBV6, respectively; human TRAV19 and human TRBV10, respectively; human TRAV19 and human TRBV7, respectively; human TRAV19 and human TRBV19, respectively; human TRAV19 and human TRBV20, respectively; human TRAV19 and human TRBV28, respectively; human TRAV21 and human TRBV5, respectively; human TRAV21 and human TRBV6, respectively; human TRAV21 and human TRBV10, respectively; human TRAV21 and human TRBV7, respectively; human TRAV21 and human TRBV19, respectively; human TRAV21 and human TRBV20, respectively; human TRAV21 and human TRBV28, respectively; human TRAV30 and human TRBV5, respectively; human TRAV30 and human TRBV6, respectively; human TRAV30 and human TRBV10, respectively; human TRAV30 and human TRBV7, respectively; human TRAV30 and human TRBV19, respectively; human TRAV30 and human TRBV20, respectively; or human TRAV30 and human TRBV28, respectively.

In some embodiments, the first target molecule is human TRAV12, human TRAV13, human TRAV19, human TRAV21, or human TRAV30, and the second target molecule is human TRBV5-1, human TRBV6-5, human TRBV10-1, human TRBV10-2, human TRBV10-3, human TRBV7-2, human TRBV7-3, human TRBV19, human TRBV20-1, or human TRBV28.

In some embodiments, the first target molecule and the second target molecule are:
human TRAV12 and human TRBV5-1, respectively; human TRAV12 and human TRBV6-5, respectively; human TRAV12 and human TRBV10-1, respectively; human TRAV12 and human TRBV10-2, respectively; human TRAV12 and human TRBV10-3, respectively; human TRAV12 and human TRBV7-2, respectively; human TRAV12 and human TRBV7-3, respectively; human TRAV12 and human TRBV19, respectively; human TRAV12 and human TRBV20-1, respectively; human TRAV12 and human TRBV28, respectively; human TRAV13 and human TRBV5-1, respectively; human TRAV13 and human TRBV6-5, respectively; human TRAV13 and human TRBV10-1, respectively; human TRAV13 and human TRBV10-2, respectively; human TRAV13 and human TRBV10-3, respectively; human TRAV13 and human TRBV7-2, respectively; human TRAV13 and human TRBV7-3, respectively; human TRAV13 and human TRBV19, respectively; human TRAV13 and human TRBV20-1, respectively; human TRAV13 and human TRBV28, respectively; human TRAV19 and human TRBV5-1, respectively; human TRAV19 and human TRBV6-5, respectively; human TRAV19 and human TRBV10-1, respectively; human TRAV19 and human TRBV10-2, respectively; human TRAV19 and human TRBV10-3, respectively; human TRAV19 and human TRBV7-2, respectively; human TRAV19 and human TRBV7-3, respectively; human TRAV19 and human TRBV19, respectively; human TRAV19 and human TRBV20-1, respectively; human TRAV19 and human TRBV28, respectively; human TRAV21 and human TRBV5-1, respectively; human TRAV21 and human TRBV6-5, respectively; human TRAV21 and human TRBV10-1, respectively; human TRAV21 and human TRBV10-2, respectively; human TRAV21 and human TRBV10-3, respectively; human TRAV21 and human TRBV7-2, respectively; human TRAV21 and human TRBV7-3, respectively; human TRAV21 and human TRBV19, respectively; human TRAV21 and human TRBV20-1, respectively; human TRAV21 and human TRBV28, respectively; human TRAV30 and human TRBV5-1, respectively; human TRAV30 and human TRBV6-5, respectively; human TRAV30 and human TRBV10-1, respectively; human TRAV30 and human TRBV10-2, respectively; human TRAV30 and human TRBV10-3, respectively; human TRAV30 and human TRBV7-2, respectively; human TRAV30 and human TRBV7-3, respectively; human TRAV30 and human TRBV19, respectively; human TRAV30 and human TRBV20-1, respectively; or human TRAV30 and human TRBV28, respectively.

In some embodiments, the first target molecule is human TRAV12-2, human TRAV13-1, human TRAV19, human TRAV21, or human TRAV30, and the second target molecule is human TRBV5-1, human TRBV6-5, human TRBV10-1, human TRBV10-2, human TRBV10-3, human TRBV7-2, human TRBV7-3, human TRBV19, human TRBV20-1, or human TRBV28.

In some embodiments, the first target molecule and the second target molecule are:
human TRAV12-2 and human TRBV5-1, respectively; human TRAV12-2 and human TRBV6-5, respectively; human TRAV12-2 and human TRBV10-1, respectively; human TRAV12-2 and human TRBV10-2, respectively; human TRAV12-2 and human TRBV10-3, respectively; human TRAV12-2 and human TRBV7-2, respectively; human TRAV12-2 and human TRBV7-3, respectively; human TRAV12-2 and human TRBV19, respectively; human TRAV12-2 and human TRBV20-1, respectively; human TRAV12-2 and human TRBV28, respectively; human TRAV13-1 and human TRBV5-1, respectively;
human TRAV13-1 and human TRBV6-5, respectively; human TRAV13-1 and human TRBV10-1, respectively; human TRAV13-1 and human TRBV10-2, respectively; human TRAV13-1 and human TRBV10-3, respectively; human TRAV13-1 and human TRBV7-2, respectively; human TRAV13-1 and human TRBV7-3, respectively; human TRAV13-1 and human TRBV19, respectively; human TRAV13-1 and human TRBV20-1, respectively; human TRAV13-1 and human TRBV28, respectively; human TRAV19 and human TRBV5-1, respectively; human TRAV19 and human TRBV6-5, respectively; human TRAV19 and human TRBV10-1, respectively; human TRAV19 and human TRBV10-2, respectively; human TRAV19 and human TRBV10-3, respectively; human TRAV19 and human TRBV7-2, respectively; human TRAV19 and human TRBV7-3, respectively; human TRAV19 and human TRBV19, respectively; human TRAV19 and human TRBV20-1, respectively; human TRAV19 and human TRBV28, respectively; human TRAV21 and human TRBV5-1, respectively; human TRAV21 and human TRBV6-5, respectively; human TRAV21 and human TRBV10-1, respectively; human TRAV21 and human TRBV10-2, respectively; human TRAV21 and human TRBV10-3, respectively; human TRAV21 and human TRBV7-2, respectively; human TRAV21 and human TRBV7-3, respectively; human TRAV21 and human TRBV19, respectively; human TRAV21 and human TRBV20-1, respectively; human TRAV21 and human TRBV28, respectively; human TRAV30 and human TRBV5-1, respectively; human TRAV30 and human TRBV6-5, respectively; human TRAV30 and human TRBV10-1, respectively; human TRAV30 and human TRBV10-2, respectively; human TRAV30 and human TRBV10-3, respectively; human TRAV30 and human TRBV7-2, respectively; human TRAV30 and human TRBV7-3, respectively; human TRAV30 and human TRBV19, respectively; human TRAV30 and human TRBV20-1, respectively; or human TRAV30 and human TRBV28, respectively.

In some embodiments, the first target molecule is human TCRβV27, and the second target molecule is human TCRβV6.

In some embodiments, the first binder comprises a full antibody or antigen-binding fragment thereof, a bivalent antibody, bispecific antibody, or a biparatopic antibody.

In some embodiments, the second binder comprises a full antibody or antigen-binding fragment thereof, a bivalent antibody, bispecific antibody, or a biparatopic antibody.

In some embodiments, the antigen-binding fragment is a Fab, F(ab′)2, Fv, a single chain Fv (scFv), a single domain antibody, a diabody, a single domain antibody, or a camelid antibody.

In some embodiments, the first binder comprises a heavy chain variable region (VH) comprising a heavy chain complementarity determining region 3 (HC CDR3) comprising the sequence of SEQ ID NO: 3343, SEQ ID NO: 3323, SEQ ID NO: 3353, SEQ ID NO: 1187, SEQ ID NO: 3373, SEQ ID NO: 3383, SEQ ID NO: 3393, SEQ ID NO: 3403, or SEQ ID NO: 3413.

In some embodiments, the first binder comprises a VH comprising a heavy chain complementarity determining region 1 (HC CDR1) comprising the sequence of SEQ ID NO: 3341, SEQ ID NO: 3321, SEQ ID NO: 3351, SEQ ID NO:1190, SEQ ID NO: 3371, SEQ ID NO: 3381, SEQ ID NO: 3391, SEQ ID NO: 3401, SEQ ID NO: 3411, SEQ ID NO: 3421, or SEQ ID NO: 3702.

In some embodiments, the first binder comprises a VH comprising a heavy chain complementarity determining region 2 (HC CDR2) comprising the sequence of SEQ ID NO: 3342, SEQ ID NO: 3322, SEQ ID NO: 3352, SEQ ID NO: 1186, SEQ ID NO: 3372, SEQ ID NO: 3382, SEQ ID NO: 3392, SEQ ID NO: 3402, or SEQ ID NO: 3412.

In some embodiments, the first binder comprises a VH comprising:
(i) a HC CDR1 comprising the sequence of SEQ ID NO: 3341, a HC CDR2 comprising the sequence of SEQ ID NO: 3342, and a HC CDR3 comprising the sequence of SEQ ID NO: 3343;
(ii) a HC CDR1 comprising the sequence of SEQ ID NO: 3321, a HC CDR2 comprising the sequence of SEQ ID NO: 3322, and a HC CDR3 comprising the sequence of SEQ ID NO: 3323;
(iii) a HC CDR1 comprising the sequence of SEQ ID NO: 3351, a HC CDR2 comprising the sequence of SEQ ID NO: 3352, and a HC CDR3 comprising the sequence of SEQ ID NO: 3353;
(iv) a HC CDR1 comprising the sequence of SEQ ID NO: 1190, a HC CDR2 comprising the sequence of SEQ ID NO: 1186, and a HC CDR3 comprising the sequence of SEQ ID NO: 1187;
(v) a HC CDR1 comprising the sequence of SEQ ID NO: 3371, a HC CDR2 comprising the sequence of SEQ ID NO: 3372, and a HC CDR3 comprising the sequence of SEQ ID NO: 3373;
(vi) a HC CDR1 comprising the sequence of SEQ ID NO: 3381, a HC CDR2 comprising the sequence of SEQ ID NO: 3382, and a HC CDR3 comprising the sequence of SEQ ID NO: 3383;
(vii) a HC CDR1 comprising the sequence of SEQ ID NO: 3391, a HC CDR2 comprising the sequence of SEQ ID NO: 3392, and a HC CDR3 comprising the sequence of SEQ ID NO: 3393;
(viii) a HC CDR1 comprising the sequence of SEQ ID NO: 3401, a HC CDR2 comprising the sequence of SEQ ID NO: 3402, and a HC CDR3 comprising the sequence of SEQ ID NO: 3403;
(ix) a HC CDR1 comprising the sequence of SEQ ID NO: 3351, SEQ ID NO: 3421, or SEQ ID NO: 3702, a HC CDR2 comprising the sequence of SEQ ID NO: 3352, and a HC CDR3 comprising the sequence of SEQ ID NO: 3353; or
(x) a HC CDR1 comprising the sequence of SEQ ID NO: 3411, a HC CDR2 comprising the sequence of SEQ ID NO: 3412, and a HC CDR3 comprising the sequence of SEQ ID NO: 3413.

In some embodiments, the first binder comprises a light chain variable region (VL) comprising a light chain complementarity determining region 3 (LC CDR3) comprising the sequence of SEQ ID NO: 3346, SEQ ID NO: 3326, SEQ ID NO: 3356, SEQ ID NO: 1193, SEQ ID NO: 3376, SEQ ID NO: 3386, SEQ ID NO: 3396, SEQ ID NO: 3406, or SEQ ID NO: 3416.

In some embodiments, the first binder comprises a VL comprising a light chain complementarity determining region 1 (LC CDR1) comprising the sequence of SEQ ID NO: 3344, SEQ ID NO: 3324, SEQ ID NO: 3354, SEQ ID NO: 1191, SEQ ID NO: 3374, SEQ ID NO: 3384, SEQ ID NO: 3394, SEQ ID NO: 3404, or SEQ ID NO: 3414.

In some embodiments, the first binder comprises a VL comprising a light chain complementarity determining region 2 (LC CDR2) comprising the sequence of SEQ ID NO: 3345, SEQ ID NO: 3325, SEQ ID NO: 3355, SEQ ID NO:1192, SEQ ID NO: 3375, SEQ ID NO: 3395, SEQ ID NO: 3405, SEQ ID NO: 3415, or SEQ ID NO: 3443.

In some embodiments, the first binder comprises a VL comprising:
(i) a LC CDR1 comprising the sequence of SEQ ID NO: 3344, a LC CDR2 comprising the sequence of SEQ ID NO: 3345, and a LC CDR3 comprising the sequence of SEQ ID NO: 3346;
(ii) a LC CDR1 comprising the sequence of SEQ ID NO: 3324, a LC CDR2 comprising the sequence of SEQ ID NO: 3325, and a LC CDR3 comprising the sequence of SEQ ID NO: 3326;
(iii) a LC CDR1 comprising the sequence of SEQ ID NO: 3354, a LC CDR2 comprising the sequence of SEQ ID NO: 3355, and a LC CDR3 comprising the sequence of SEQ ID NO: 3356;
(iv) a LC CDR1 comprising the sequence of SEQ ID NO: 1191, a LC CDR2 comprising the sequence of SEQ ID NO: 1192, and a LC CDR3 comprising the sequence of SEQ ID NO: 1193;
(v) a LC CDR1 comprising the sequence of SEQ ID NO: 3374, a LC CDR2 comprising the sequence of SEQ ID NO: 3375, and a LC CDR3 comprising the sequence of SEQ ID NO: 3376;
(vi) a LC CDR1 comprising the sequence of SEQ ID NO: 3384, a LC CDR2 comprising the sequence of SEQ ID NO: 3325, and a LC CDR3 comprising the sequence of SEQ ID NO: 3386;
(vii) a LC CDR1 comprising the sequence of SEQ ID NO: 3394, a LC CDR2 comprising the sequence of SEQ ID NO: 3395, and a LC CDR3 comprising the sequence of SEQ ID NO: 3396;
(viii) a LC CDR1 comprising the sequence of SEQ ID NO: 3404, a LC CDR2 comprising the sequence of SEQ ID NO: 3405, and a LC CDR3 comprising the sequence of SEQ ID NO: 3406;
(ix) a LC CDR1 comprising the sequence of SEQ ID NO: 3414, a LC CDR2 comprising the sequence of SEQ ID NO: 3415, and a LC CDR3 comprising the sequence of SEQ ID NO: 3416; or
(x) a LC CDR1 comprising the sequence of SEQ ID NO: 3354, a LC CDR2 comprising the sequence of SEQ ID NO: 3443, and a LC CDR3 comprising the sequence of SEQ ID NO: 3356.

In some embodiments, the first binder comprises:
(i) a VH comprising a HC CDR1 comprising the sequence of SEQ ID NO: 3341, a HC CDR2 comprising the sequence of SEQ ID NO: 3342, a HC CDR3 comprising the sequence of SEQ ID NO: 3343, and a VL comprising a LC CDR1 comprising the sequence of SEQ ID NO: 3344, a LC CDR2 comprising the sequence of SEQ ID NO: 3345, and a LC CDR3 comprising the sequence of SEQ ID NO: 3346;
(ii) a VH comprising a HC CDR1 comprising the sequence of SEQ ID NO: 3321, a HC CDR2 comprising the sequence of SEQ ID NO: 3322, a HC CDR3 comprising the sequence of SEQ ID NO: 3323, and a VL comprising a LC CDR1 comprising the sequence of SEQ ID NO: 3324, a LC CDR2 comprising the sequence of SEQ ID NO: 3325, and a LC CDR3 comprising the sequence of SEQ ID NO: 3326;
(iii) a VH comprising a HC CDR1 comprising the sequence of SEQ ID NO: 3351, a HC CDR2 comprising the sequence of SEQ ID NO: 3352, a HC CDR3 comprising the sequence of SEQ ID NO: 3353, and a VL comprising a LC CDR1 comprising the sequence of SEQ ID NO: 3354, a LC CDR2 comprising the sequence of SEQ ID NO: 3355, and a LC CDR3 comprising the sequence of SEQ ID NO: 3356;
(iv) a VH comprising a HC CDR1 comprising the sequence of SEQ ID NO: 1190, a HC CDR2 comprising the sequence of SEQ ID NO: 1186, a HC CDR3 comprising the sequence of SEQ ID NO: 1187, and a VL comprising a LC CDR1 comprising the sequence of SEQ ID NO: 1191, a LC CDR2 comprising the sequence of SEQ ID NO: 1192, and a LC CDR3 comprising the sequence of SEQ ID NO: 1193;
(v) a VH comprising a HC CDR1 comprising the sequence of SEQ ID NO: 3371, a HC CDR2 comprising the sequence of SEQ ID NO: 3372, a HC CDR3 comprising the sequence of SEQ ID NO: 3373, and a VL comprising a LC CDR1 comprising the sequence of SEQ ID NO: 3374, a LC CDR2 comprising the sequence of SEQ ID NO: 3375, and a LC CDR3 comprising the sequence of SEQ ID NO: 3376;
(vi) a VH comprising a HC CDR1 comprising the sequence of SEQ ID NO: 3381, a HC CDR2 comprising the sequence of SEQ ID NO: 3382, a HC CDR3 comprising the sequence of SEQ ID NO: 3383, and a VL comprising a LC CDR1 comprising the sequence of SEQ ID NO: 3384, a LC CDR2 comprising the sequence of SEQ ID NO: 3325, and a LC CDR3 comprising the sequence of SEQ ID NO: 3386;
(vii) a VH comprising a HC CDR1 comprising the sequence of SEQ ID NO: 3391, a HC CDR2 comprising the sequence of SEQ ID NO: 3392, a HC CDR3 comprising the sequence of SEQ ID NO: 3393, and a VL comprising a LC CDR1 comprising the sequence of SEQ ID NO: 3394, a LC CDR2 comprising the sequence of SEQ ID NO: 3395, and a LC CDR3 comprising the sequence of SEQ ID NO: 3396;
(viii) a VH comprising a HC CDR1 comprising the sequence of SEQ ID NO: 3401, a HC CDR2 comprising the sequence of SEQ ID NO: 3402, a HC CDR3 comprising the sequence of SEQ ID NO: 3403, and a VL comprising a LC CDR1 comprising the sequence of SEQ ID NO: 3404, a LC CDR2 comprising the sequence of SEQ ID NO: 3405, and a LC CDR3 comprising the sequence of SEQ ID NO: 3406;
(ix) a VH comprising a HC CDR1 comprising the sequence of SEQ ID NO: 3411, a HC CDR2 comprising the sequence of SEQ ID NO: 3412, a HC CDR3 comprising the sequence of SEQ ID NO: 3413, and a VL comprising a LC CDR1 comprising the sequence of SEQ ID NO: 3414, a LC CDR2 comprising the sequence of SEQ ID NO: 3415, and a LC CDR3 comprising the sequence of SEQ ID NO: 3416:
(x) a VH comprising a HC CDR1 comprising the sequence of SEQ ID NO: 3351, SEQ ID NO: 3421, or SEQ ID NO: 3702, a HC CDR2 comprising the sequence of SEQ ID NO: 3352, a HC CDR3 comprising the sequence of SEQ ID NO: 3353, and a VL comprising a LC CDR1 comprising the sequence of SEQ ID NO: 3354, a LC CDR2 comprising the sequence of SEQ ID NO: 3355, and a LC CDR3 comprising the sequence of SEQ ID NO: 3356; or
(xi) a VH comprising a HC CDR1 comprising the sequence of SEQ ID NO: 3351, SEQ ID NO: 3421, or SEQ ID NO: 3702, a HC CDR2 comprising the sequence of SEQ ID NO: 3352, a HC CDR3 comprising the sequence of SEQ ID NO: 3353, and a VL comprising a LC CDR1 comprising the sequence of SEQ ID NO: 3354, a LC CDR2 comprising the sequence of SEQ ID NO: 3443, and a LC CDR3 comprising the sequence of SEQ ID NO: 3356.

In some embodiments, the first binder comprises a VH comprising a sequence having at least 75% sequence identity to any one sequence selected from the group consisting of: SEQ ID NO: 3445, SEQ ID NO: 3448, SEQ ID NO: 3450, SEQ ID NO: 1346, SEQ ID NO: 1196, SEQ ID NO: 3455, SEQ ID NO: 3457, SEQ ID NO: 3463, SEQ ID NO: 3465, and SEQ ID NO: 3470.

In some embodiments, the first binder comprises a VH comprising any one sequence selected from the group consisting of: SEQ ID NO: 3445, SEQ ID NO: 3448, SEQ ID NO: 3450, SEQ ID NO: 1346, SEQ ID NO: 1196, SEQ ID NO: 3455, SEQ ID NO: 3457, SEQ ID NO: 3463, SEQ ID NO: 3465, and SEQ ID NO: 3470.

In some embodiments, the first binder comprises a VL comprising a sequence having at least 75% sequence identity to any one sequence selected from the group consisting of: SEQ ID NO: 3446, SEQ ID NO: 3449, SEQ ID NO: 3451, SEQ ID NO: 1349, SEQ ID NO:1195, SEQ ID NO: 3456, SEQ ID NO: 3458, SEQ ID NO: 3464, SEQ ID NO: 3466, SEQ ID NO: 3471, SEQ ID NO: 3475, and SEQ ID NO: 3476.

In some embodiments, the first binder comprises a VL comprising any one sequence selected from the group consisting of: SEQ ID NO: 3446, SEQ ID NO: 3449, SEQ ID NO: 3451, SEQ ID NO: 1349, SEQ ID NO:1195, SEQ ID NO: 3456, SEQ ID NO: 3458, SEQ ID NO: 3464, SEQ ID NO: 3466, SEQ ID NO: 3471, SEQ ID NO: 3475, and SEQ ID NO: 3476.

In some embodiments, the first binder comprises:
(i) a VH comprising a sequence having at least 75% sequence identity to any one sequence selected from the group consisting of: SEQ ID NO: 3445, SEQ ID NO: 3448, SEQ ID NO: 3450, SEQ ID NO: 1346, SEQ ID NO: 1196, SEQ ID NO: 3455, SEQ ID NO: 3457, SEQ ID NO: 3463, SEQ ID NO: 3465, and SEQ ID NO: 3470; and
(ii) a VL comprising a sequence having at least 75% sequence identity to any one sequence selected from the group consisting of: SEQ ID NO: 3446, SEQ ID NO: 3449, SEQ ID NO: 3451, SEQ ID NO: 1349, SEQ ID NO:1195, SEQ ID NO: 3456, SEQ ID NO: 3458, SEQ ID NO: 3464, SEQ ID NO: 3466, SEQ ID NO: 3471, SEQ ID NO: 3475, and SEQ ID NO: 3476.

In some embodiments, the first binder comprises:
(i) a VH comprising any one sequence selected from the group consisting of: SEQ ID NO: 3445, SEQ ID NO: 3448, SEQ ID NO: 3450, SEQ ID NO: 1346, SEQ ID NO: 1196, SEQ ID NO: 3455, SEQ ID NO: 3457, SEQ ID NO: 3463, SEQ ID NO: 3465, and SEQ ID NO: 3470; and
(ii) a VL comprising any one sequence selected from the group consisting of: SEQ ID NO: 3446, SEQ ID NO: 3449, SEQ ID NO: 3451, SEQ ID NO: 1349, SEQ ID NO:1195, SEQ ID NO: 3456, SEQ ID NO: 3458, SEQ ID NO: 3464, SEQ ID NO: 3466, SEQ ID NO: 3471, SEQ ID NO: 3475, and SEQ ID NO: 3476.

In some embodiments, the first binder comprises:
(i) a VH comprising a sequence having at least 75% sequence identity to the sequence of SEQ ID NO: 3445, and a VL comprising a sequence having at least 75% sequence identity to the sequence of SEQ ID NO: 3446;
(ii) a VH comprising a sequence having at least 75% sequence identity to the sequence of SEQ ID NO: 3448, and a VL comprising a sequence having at least 75% sequence identity to the sequence of SEQ ID NO: 3449;
(iii) a VH comprising a sequence having at least 75% sequence identity to the sequence of SEQ ID NO: 3450, and a VL comprising a sequence having at least 75% sequence identity to the sequence of SEQ ID NO: 3451;
(iv) a VH comprising a sequence having at least 75% sequence identity to the sequence of SEQ ID NO: 1346, and a VL comprising a sequence having at least 75% sequence identity to the sequence of SEQ ID NO: 1349;
(v) a VH comprising a sequence having at least 75% sequence identity to the sequence of SEQ ID NO: 1196, and a VL comprising a sequence having at least 75% sequence identity to the sequence of SEQ ID NO: 1195;
(vi) a VH comprising a sequence having at least 75% sequence identity to the sequence of SEQ ID NO: 3455, and a VL comprising a sequence having at least 75% sequence identity to the sequence of SEQ ID NO: 3456;
(vii) a VH comprising a sequence having at least 75% sequence identity to the sequence of SEQ ID NO: 3457, and a VL comprising a sequence having at least 75% sequence identity to the sequence of SEQ ID NO: 3458;
(viii) a VH comprising a sequence having at least 75% sequence identity to the sequence of SEQ ID NO: 3463, and a VL comprising a sequence having at least 75% sequence identity to the sequence of SEQ ID NO: 3464;
(ix) a VH comprising a sequence having at least 75% sequence identity to the sequence of SEQ ID NO: 3465, and a VL comprising a sequence having at least 75% sequence identity to the sequence of SEQ ID NO: 3466;
(x) a VH comprising a sequence having at least 75% sequence identity to the sequence of SEQ ID NO: 3470, and a VL comprising a sequence having at least 75% sequence identity to the sequence of SEQ ID NO: 3471;
(xi) a VH comprising a sequence having at least 75% sequence identity to the sequence of SEQ ID NO: 1346, and a VL comprising a sequence having at least 75% sequence identity to the sequence of SEQ ID NO: 3475; or
(xii) a VH comprising a sequence having at least 75% sequence identity to the sequence of SEQ ID NO: 1346, and a VL comprising a sequence having at least 75% sequence identity to the sequence of SEQ ID NO: 3476.

In some embodiments, the first binder comprises:
(i) a VH comprising the sequence of SEQ ID NO: 3445, and a VL comprising the sequence of SEQ ID NO: 3446;
(ii) a VH comprising the sequence of SEQ ID NO: 3448, and a VL comprising the sequence of SEQ ID NO: 3449;
(iii) a VH comprising the sequence of SEQ ID NO: 3450, and a VL comprising the sequence of SEQ ID NO: 3451;
(iv) a VH comprising the sequence of SEQ ID NO: 1346, and a VL comprising the sequence of SEQ ID NO: 1349;
(v) a VH comprising the sequence of SEQ ID NO: 1196, and a VL comprising the sequence of SEQ ID NO: 1195;
(vi) a VH comprising the sequence of SEQ ID NO: 3455, and a VL comprising the sequence of SEQ ID NO: 3456;
(vii) a VH comprising the sequence of SEQ ID NO: 3457, and a VL comprising the sequence of SEQ ID NO: 3458;
(viii) a VH comprising the sequence of SEQ ID NO: 3463, and a VL comprising the sequence of SEQ ID NO: 3464;
(ix) a VH comprising the sequence of SEQ ID NO: 3465, and a VL comprising the sequence of SEQ ID NO: 3466;
(x) a VH comprising the sequence of SEQ ID NO: 3470, and a VL comprising the sequence of SEQ ID NO: 3471;
(xi) a VH comprising the sequence of SEQ ID NO: 1346, and a VL comprising the sequence of SEQ ID NO: 3475; or
(xii) a VH comprising the sequence of SEQ ID NO: 1346, and a VL comprising the sequence of SEQ ID NO: 3476.

In some embodiments, the second binder comprises a heavy chain variable region (VH) comprising a heavy chain complementarity determining region 3 (HC CDR3) comprising the sequence of SEQ ID NO: 3343, SEQ ID NO: 3323, SEQ ID NO: 3353, SEQ ID NO: 1187, SEQ ID NO: 3373, SEQ ID NO: 3383, SEQ ID NO: 3393, SEQ ID NO: 3403, or SEQ ID NO: 3413.

In some embodiments, the second binder comprises a VH comprising a heavy chain complementarity determining region 1 (HC CDR1) comprising the sequence of SEQ ID NO: 3341, SEQ ID NO: 3321, SEQ ID NO: 3351, SEQ ID NO:1190, SEQ ID NO: 3371, SEQ ID NO: 3381, SEQ ID NO: 3391, SEQ ID NO: 3401, SEQ ID NO: 3411, SEQ ID NO: 3421, or SEQ ID NO: 3702.

In some embodiments, the second binder comprises a VH comprising a heavy chain complementarity determining region 2 (HC CDR2) comprising the sequence of SEQ ID NO: 3342, SEQ ID NO: 3322, SEQ ID NO: 3352, SEQ ID NO: 1186, SEQ ID NO: 3372, SEQ ID NO: 3382, SEQ ID NO: 3392, SEQ ID NO: 3402, or SEQ ID NO: 3412.

In some embodiments, the second binder comprises a VH comprising:
(i) a HC CDR1 comprising the sequence of SEQ ID NO: 3341, a HC CDR2 comprising the sequence of SEQ ID NO: 3342, and a HC CDR3 comprising the sequence of SEQ ID NO: 3343;
(ii) a HC CDR1 comprising the sequence of SEQ ID NO: 3321, a HC CDR2 comprising the sequence of SEQ ID NO: 3322, and a HC CDR3 comprising the sequence of SEQ ID NO: 3323;
(iii) a HC CDR1 comprising the sequence of SEQ ID NO: 3351, a HC CDR2 comprising the sequence of SEQ ID NO: 3352, and a HC CDR3 comprising the sequence of SEQ ID NO: 3353;
(vi) a HC CDR1 comprising the sequence of SEQ ID NO: 1190, a HC CDR2 comprising the sequence of SEQ ID NO: 1186, and a HC CDR3 comprising the sequence of SEQ ID NO: 1187;
(v) a HC CDR1 comprising the sequence of SEQ ID NO: 3371, a HC CDR2 comprising the sequence of SEQ ID NO: 3372, and a HC CDR3 comprising the sequence of SEQ ID NO: 3373;
(vi) a HC CDR1 comprising the sequence of SEQ ID NO: 3381, a HC CDR2 comprising the sequence of SEQ ID NO: 3382, and a HC CDR3 comprising the sequence of SEQ ID NO: 3383;
(vii) a HC CDR1 comprising the sequence of SEQ ID NO: 3391, a HC CDR2 comprising the sequence of SEQ ID NO: 3392, and a HC CDR3 comprising the sequence of SEQ ID NO: 3393;
(viii) a HC CDR1 comprising the sequence of SEQ ID NO: 3401, a HC CDR2 comprising the sequence of SEQ ID NO: 3402, and a HC CDR3 comprising the sequence of SEQ ID NO: 3403;
(ix) a HC CDR1 comprising the sequence of SEQ ID NO: 3351, SEQ ID NO: 3421, or SEQ ID NO: 3702, a HC CDR2 comprising the sequence of SEQ ID NO: 3352, and a HC CDR3 comprising the sequence of SEQ ID NO: 3353; or
(x) a HC CDR1 comprising the sequence of SEQ ID NO: 3411, a HC CDR2 comprising the sequence of SEQ ID NO: 3412, and a HC CDR3 comprising the sequence of SEQ ID NO: 3413.

In some embodiments, the second binder comprises a light chain variable region (VL) comprising a light chain complementarity determining region 3 (LC CDR3) comprising the sequence of SEQ ID NO: 3346, SEQ ID NO: 3326, SEQ ID NO: 3356, SEQ ID NO: 1193, SEQ ID NO: 3376, SEQ ID NO: 3386, SEQ ID NO: 3396, SEQ ID NO: 3406, or SEQ ID NO: 3416.

In some embodiments, the second binder comprises a VL comprising a light chain complementarity determining region 1 (LC CDR1) comprising the sequence of SEQ ID NO: 3344, SEQ ID NO: 3324, SEQ ID NO: 3354, SEQ ID NO: 1191, SEQ ID NO: 3374, SEQ ID NO: 3384, SEQ ID NO: 3394, SEQ ID NO: 3404, or SEQ ID NO: 3414.

In some embodiments, the second binder comprises a VL comprising a light chain complementarity determining region 2 (LC CDR2) comprising the sequence of SEQ ID NO: 3345, SEQ ID NO: 3325, SEQ ID NO: 3355, SEQ ID NO:1192, SEQ ID NO: 3375, SEQ ID NO: 3395, SEQ ID NO: 3405, SEQ ID NO: 3415, or SEQ ID NO: 3443.

In some embodiments, the second binder comprises a VL comprising:
(i) a LC CDR1 comprising the sequence of SEQ ID NO: 3344, a LC CDR2 comprising the sequence of SEQ ID NO: 3345, and a LC CDR3 comprising the sequence of SEQ ID NO: 3346;
(ii) a LC CDR1 comprising the sequence of SEQ ID NO: 3324, a LC CDR2 comprising the sequence of SEQ ID NO: 3325, and a LC CDR3 comprising the sequence of SEQ ID NO: 3326;
(iii) a LC CDR1 comprising the sequence of SEQ ID NO: 3354, a LC CDR2 comprising the sequence of SEQ ID NO: 3355, and a LC CDR3 comprising the sequence of SEQ ID NO: 3356;
(iv) a LC CDR1 comprising the sequence of SEQ ID NO: 1191, a LC CDR2 comprising the sequence of SEQ ID NO: 1192, and a LC CDR3 comprising the sequence of SEQ ID NO: 1193;
(v) a LC CDR1 comprising the sequence of SEQ ID NO: 3374, a LC CDR2 comprising the sequence of SEQ ID NO: 3375, and a LC CDR3 comprising the sequence of SEQ ID NO: 3376;
(vi) a LC CDR1 comprising the sequence of SEQ ID NO: 3384, a LC CDR2 comprising the sequence of SEQ ID NO: 3325, and a LC CDR3 comprising the sequence of SEQ ID NO: 3386;
(vii) a LC CDR1 comprising the sequence of SEQ ID NO: 3394, a LC CDR2 comprising the sequence of SEQ ID NO: 3395, and a LC CDR3 comprising the sequence of SEQ ID NO: 3396;
(viii) a LC CDR1 comprising the sequence of SEQ ID NO: 3404, a LC CDR2 comprising the sequence of SEQ ID NO: 3405, and a LC CDR3 comprising the sequence of SEQ ID NO: 3406;
(ix) a LC CDR1 comprising the sequence of SEQ ID NO: 3414, a LC CDR2 comprising the sequence of SEQ ID NO: 3415, and a LC CDR3 comprising the sequence of SEQ ID NO: 3416; or
(x) a LC CDR1 comprising the sequence of SEQ ID NO: 3354, a LC CDR2 comprising the sequence of SEQ ID NO: 3443, and a LC CDR3 comprising the sequence of SEQ ID NO: 3356.

In some embodiments, the second binder comprises:
(i) a VH comprising a HC CDR1 comprising the sequence of SEQ ID NO: 3341, a HC CDR2 comprising the sequence of SEQ ID NO: 3342, a HC CDR3 comprising the sequence of SEQ ID NO: 3343, and a VL comprising a LC CDR1 comprising the sequence of SEQ ID NO: 3344, a LC CDR2 comprising the sequence of SEQ ID NO: 3345, and a LC CDR3 comprising the sequence of SEQ ID NO: 3346;
(ii) a VH comprising a HC CDR1 comprising the sequence of SEQ ID NO: 3321, a HC CDR2 comprising the sequence of SEQ ID NO: 3322, a HC CDR3 comprising the sequence of SEQ ID NO: 3323, and a VL comprising a LC CDR1 comprising the sequence of SEQ ID NO: 3324, a LC CDR2 comprising the sequence of SEQ ID NO: 3325, and a LC CDR3 comprising the sequence of SEQ ID NO: 3326;
(iii) a VH comprising a HC CDR1 comprising the sequence of SEQ ID NO: 3351, a HC CDR2 comprising the sequence of SEQ ID NO: 3352, a HC CDR3 comprising the sequence of SEQ ID NO: 3353, and a VL comprising a LC CDR1 comprising the sequence of SEQ ID NO: 3354, a LC CDR2 comprising the sequence of SEQ ID NO: 3355, and a LC CDR3 comprising the sequence of SEQ ID NO: 3356;
(iv) a VH comprising a HC CDR1 comprising the sequence of SEQ ID NO: 1190, a HC CDR2 comprising the sequence of SEQ ID NO: 1186, a HC CDR3 comprising the sequence of SEQ ID NO: 1187, and a VL comprising a LC CDR1 comprising the sequence of SEQ ID NO: 1191, a LC CDR2 comprising the sequence of SEQ ID NO: 1192, and a LC CDR3 comprising the sequence of SEQ ID NO: 1193;
(v) a VH comprising a HC CDR1 comprising the sequence of SEQ ID NO: 3371, a HC CDR2 comprising the sequence of SEQ ID NO: 3372, a HC CDR3 comprising the sequence of SEQ ID NO: 3373, and a VL comprising a LC CDR1 comprising the sequence of SEQ ID NO: 3374, a LC CDR2 comprising the sequence of SEQ ID NO: 3375, and a LC CDR3 comprising the sequence of SEQ ID NO: 3376;
(vi) a VH comprising a HC CDR1 comprising the sequence of SEQ ID NO: 3381, a HC CDR2 comprising the sequence of SEQ ID NO: 3382, a HC CDR3 comprising the sequence of SEQ ID NO: 3383, and a VL comprising a LC CDR1 comprising the sequence of SEQ ID NO: 3384, a LC CDR2 comprising the sequence of SEQ ID NO: 3325, and a LC CDR3 comprising the sequence of SEQ ID NO: 3386;
(vii) a VH comprising a HC CDR1 comprising the sequence of SEQ ID NO: 3391, a HC CDR2 comprising the sequence of SEQ ID NO: 3392, a HC CDR3 comprising the sequence of SEQ ID NO: 3393, and a VL comprising a LC CDR1 comprising the sequence of SEQ ID NO: 3394, a LC CDR2 comprising the sequence of SEQ ID NO: 3395, and a LC CDR3 comprising the sequence of SEQ ID NO: 3396;
(viii) a VH comprising a HC CDR1 comprising the sequence of SEQ ID NO: 3401, a HC CDR2 comprising the sequence of SEQ ID NO: 3402, a HC CDR3 comprising the sequence of SEQ ID NO: 3403, and a VL comprising a LC CDR1 comprising the sequence of SEQ ID NO: 3404, a LC CDR2 comprising the sequence of SEQ ID NO: 3405, and a LC CDR3 comprising the sequence of SEQ ID NO: 3406;
(ix) a VH comprising a HC CDR1 comprising the sequence of SEQ ID NO: 3411, a HC CDR2 comprising the sequence of SEQ ID NO: 3412, a HC CDR3 comprising the sequence of SEQ ID NO: 3413, and a VL comprising a LC CDR1 comprising the sequence of SEQ ID NO: 3414, a LC CDR2 comprising the sequence of SEQ ID NO: 3415, and a LC CDR3 comprising the sequence of SEQ ID NO: 3416;
(x) a VH comprising a HC CDR1 comprising the sequence of SEQ ID NO: 3351, SEQ ID NO: 3421, or SEQ ID NO: 3702, a HC CDR2 comprising the sequence of SEQ ID NO: 3352, a HC CDR3 comprising the sequence of SEQ ID NO: 3353, and a VL comprising a LC CDR1 comprising the sequence of SEQ ID NO: 3354, a LC CDR2 comprising the sequence of SEQ ID NO: 3355, and a LC CDR3 comprising the sequence of SEQ ID NO: 3356; or
(xi) a VH comprising a HC CDR1 comprising the sequence of SEQ ID NO: 3351, SEQ ID NO: 3421, or SEQ ID NO: 3702, a HC CDR2 comprising the sequence of SEQ ID NO: 3352, a HC CDR3 comprising the sequence of SEQ ID NO: 3353, and a VL comprising a LC CDR1 comprising the sequence of SEQ ID NO: 3354, a LC CDR2 comprising the sequence of SEQ ID NO: 3443, and a LC CDR3 comprising the sequence of SEQ ID NO: 3356.

In some embodiments, the second binder comprises a VH comprising a sequence having at least 75% sequence identity to any one sequence selected from the group consisting of: SEQ ID NO: 3445, SEQ ID NO: 3448, SEQ ID NO: 3450, SEQ ID NO: 1346, SEQ ID NO: 1196, SEQ ID NO: 3455, SEQ ID NO: 3457, SEQ ID NO: 3463, SEQ ID NO: 3465, and SEQ ID NO: 3470.

In some embodiments, the second binder comprises a VH comprising any one sequence selected from the group consisting of: SEQ ID NO: 3445, SEQ ID NO: 3448, SEQ ID NO: 3450, SEQ ID NO: 1346, SEQ ID NO: 1196, SEQ ID NO: 3455, SEQ ID NO: 3457, SEQ ID NO: 3463, SEQ ID NO: 3465, and SEQ ID NO: 3470.

In some embodiments, the second binder comprises a VL comprising a sequence having at least 75% sequence identity to any one sequence selected from the group consisting of: SEQ ID NO: 3446, SEQ ID NO: 3449, SEQ ID NO: 3451, SEQ ID NO: 1349, SEQ ID NO:1195, SEQ ID NO: 3456, SEQ ID NO: 3458, SEQ ID NO: 3464, SEQ ID NO: 3466, SEQ ID NO: 3471, SEQ ID NO: 3475, and SEQ ID NO: 3476.

In some embodiments, the second binder comprises a VL comprising any one sequence selected from the group consisting of: SEQ ID NO: 3446, SEQ ID NO: 3449, SEQ ID NO: 3451, SEQ ID NO: 1349, SEQ ID NO:1195, SEQ ID NO: 3456, SEQ ID NO: 3458, SEQ ID NO: 3464, SEQ ID NO: 3466, SEQ ID NO: 3471, SEQ ID NO: 3475, and SEQ ID NO: 3476.

In some embodiments, the second binder comprises:
(i) a VH comprising a sequence having at least 75% sequence identity to any one sequence selected from the group consisting of: SEQ ID NO: 3445, SEQ ID NO: 3448, SEQ ID NO: 3450, SEQ ID NO: 1346, SEQ ID NO: 1196, SEQ ID NO: 3455, SEQ ID NO: 3457, SEQ ID NO: 3463, SEQ ID NO: 3465, and SEQ ID NO: 3470; and
(ii) a VL comprising a sequence having at least 75% sequence identity to any one sequence selected from the group consisting of: SEQ ID NO: 3446, SEQ ID NO: 3449, SEQ ID NO: 3451, SEQ ID NO: 1349, SEQ ID NO:1195, SEQ ID NO: 3456, SEQ ID NO: 3458, SEQ ID NO: 3464, SEQ ID NO: 3466, SEQ ID NO: 3471, SEQ ID NO: 3475, and SEQ ID NO: 3476.

In some embodiments, the second binder comprises:
(i) a VH comprising any one sequence selected from the group consisting of: SEQ ID NO: 3445, SEQ ID NO: 3448, SEQ ID NO: 3450, SEQ ID NO: 1346, SEQ ID NO: 1196, SEQ ID NO: 3455, SEQ ID NO: 3457, SEQ ID NO: 3463, SEQ ID NO: 3465, and SEQ ID NO: 3470; and
(ii) a VL comprising any one sequence selected from the group consisting of: SEQ ID NO: 3446, SEQ ID NO: 3449, SEQ ID NO: 3451, SEQ ID NO: 1349, SEQ ID NO: 1195, SEQ ID NO: 3456, SEQ ID NO: 3458, SEQ ID NO: 3464, SEQ ID NO: 3466, SEQ ID NO: 3471, SEQ ID NO: 3475, and SEQ ID NO: 3476.

In some embodiments, the second binder comprises:
(i) a VH comprising a sequence having at least 75% sequence identity to the sequence of SEQ ID NO: 3445, and a VL comprising a sequence having at least 75% sequence identity to the sequence of SEQ ID NO: 3446;
(ii) a VH comprising a sequence having at least 75% sequence identity to the sequence of SEQ ID NO: 3448, and a VL comprising a sequence having at least 75% sequence identity to the sequence of SEQ ID NO: 3449;
(iii) a VH comprising a sequence having at least 75% sequence identity to the sequence of SEQ ID NO: 3450, and a VL comprising a sequence having at least 75% sequence identity to the sequence of SEQ ID NO: 3451;
(iv) a VH comprising a sequence having at least 75% sequence identity to the sequence of SEQ ID NO: 1346, and a VL comprising a sequence having at least 75% sequence identity to the sequence of SEQ ID NO: 1349;
(v) a VH comprising a sequence having at least 75% sequence identity to the sequence of SEQ ID NO: 1196, and a VL comprising a sequence having at least 75% sequence identity to the sequence of SEQ ID NO: 1195;
(vi) a VH comprising a sequence having at least 75% sequence identity to the sequence of SEQ ID NO: 3455, and a VL comprising a sequence having at least 75% sequence identity to the sequence of SEQ ID NO: 3456;
(vii) a VH comprising a sequence having at least 75% sequence identity to the sequence of SEQ ID NO: 3457, and a VL comprising a sequence having at least 75% sequence identity to the sequence of SEQ ID NO: 3458;
(viii) a VH comprising a sequence having at least 75% sequence identity to the sequence of SEQ ID NO: 3463, and a VL comprising a sequence having at least 75% sequence identity to the sequence of SEQ ID NO: 3464;
(ix) a VH comprising a sequence having at least 75% sequence identity to the sequence of SEQ ID NO: 3465, and a VL comprising a sequence having at least 75% sequence identity to the sequence of SEQ ID NO: 3466;
(x) a VH comprising a sequence having at least 75% sequence identity to the sequence of SEQ ID NO: 3470, and a VL comprising a sequence having at least 75% sequence identity to the sequence of SEQ ID NO: 3471;
(xi) a VH comprising a sequence having at least 75% sequence identity to the sequence of SEQ ID NO: 1346, and a VL comprising a sequence having at least 75% sequence identity to the sequence of SEQ ID NO: 3475; or
(xii) a VH comprising a sequence having at least 75% sequence identity to the sequence of SEQ ID NO: 1346, and a VL comprising a sequence having at least 75% sequence identity to the sequence of SEQ ID NO: 3476.

In some embodiments, the second binder comprises:
(i) a VH comprising the sequence of SEQ ID NO: 3445, and a VL comprising the sequence of SEQ ID NO: 3446;
(ii) a VH comprising the sequence of SEQ ID NO: 3448, and a VL comprising the sequence of SEQ ID NO: 3449;
(iii) a VH comprising the sequence of SEQ ID NO: 3450, and a VL comprising the sequence of SEQ ID NO: 3451;
(iv) a VH comprising the sequence of SEQ ID NO: 1346, and a VL comprising the sequence of SEQ ID NO: 1349;
(v) a VH comprising the sequence of SEQ ID NO: 1196, and a VL comprising the sequence of SEQ ID NO: 1195;
(vi) a VH comprising the sequence of SEQ ID NO: 3455, and a VL comprising the sequence of SEQ ID NO: 3456;
(vii) a VH comprising the sequence of SEQ ID NO: 3457, and a VL comprising the sequence of SEQ ID NO: 3458;
(viii) a VH comprising the sequence of SEQ ID NO: 3463, and a VL comprising the sequence of SEQ ID NO: 3464;
(ix) a VH comprising the sequence of SEQ ID NO: 3465, and a VL comprising the sequence of SEQ ID NO: 3466;
(x) a VH comprising the sequence of SEQ ID NO: 3470, and a VL comprising the sequence of SEQ ID NO: 3471;
(xi) a VH comprising the sequence of SEQ ID NO: 1346, and a VL comprising the sequence of SEQ ID NO: 3475; or
(xii) a VH comprising the sequence of SEQ ID NO: 1346, and a VL comprising the sequence of SEQ ID NO: 3476.

In some embodiments, the first binder and the second binder bind to different TCRβV regions.

In some embodiments, the first binder and the second binder bind to different TCRβVs belonging to different subfamilies.

In some embodiments, the first binder and the second binder bind to different members of the same TCRβV subfamily.

In some embodiments, the first binder comprises one or more heavy chain constant regions selected from the group consisting of IgG1 heavy chain constant region or fragment thereof, IgG2 heavy chain constant region or fragment thereof. IgG3 heavy chain constant region or fragment thereof, IgGA1 heavy chain constant region or fragment thereof, IgGA2 heavy chain constant region or fragment thereof, IgG4 heavy chain constant region or fragment thereof, IgJ heavy chain constant region or fragment thereof, IgM heavy chain constant region or fragment thereof, IgD heavy chain constant region or fragment thereof, and IgE heavy chain constant region or fragment thereof.

In some embodiments, the first binder comprises a kappa light chain constant region or fragment thereof, a lambda light chain constant region or fragment thereof, or a combination thereof.

In some embodiments, the second binder comprises one or more heavy chain constant regions selected from the group consisting of IgG1 heavy chain constant region or fragment thereof, IgG2 heavy chain constant region or fragment thereof, IgG3 heavy chain constant region or fragment thereof, IgGA1 heavy chain constant region or fragment thereof, IgGA2 heavy chain constant region or fragment thereof, IgG4 heavy chain constant region or fragment thereof. IgJ heavy chain constant region or fragment thereof, IgM heavy chain constant region or fragment thereof, IgD heavy chain constant region or fragment thereof, and IgE heavy chain constant region or fragment thereof.

In some embodiments, the second binder comprises a kappa light chain constant region or fragment thereof, a lambda light chain constant region or fragment thereof, or a combination thereof.

In some embodiments, the first agent is a multifunctional molecule.

In some embodiments, the first agent is a multispecific molecule.

In some embodiments, the second agent is a multifunctional molecule.

In some embodiments, the second agent is a multispecific molecule.

In some embodiments, the first agent, the second agent, or a combination thereof comprises at least two non-contiguous polypeptide chains;
wherein a first polypeptide chain of the at least two non-contiguous polypeptide chains comprises a first Fc region, and a second polypeptide chain of the at least two non-contiguous polypeptide chains comprises a second Fc region; and
wherein the first Fc region and the second Fc region comprise an Fc interface with a knob-in-a hole.

In some embodiments, the first Fc region, the second Fc region, or a combination thereof comprises an Asn297Ala mutation, a Leu234Ala/Leu235Ala mutation, or a combination thereof.

In some embodiments, the first agent is a multispecific molecule that further comprises one or more of a tumor-targeting moiety, a cytokine molecule, a stromal modifying moiety, or an immune cell engager.

In some embodiments, the second agent is a multispecific molecule that further comprises one or more of a tumor-targeting moiety, a cytokine molecule, a stromal modifying moiety, or an immune cell engager.

In some embodiments, the first agent comprises the following con-figuration:

A-,B-[dimerization module]-C,-D,

wherein:
(a) the dimerization module comprises a first immunoglobulin chain constant region and a second immunoglobulin chain constant region;
(b) A and C are linked to the first immunoglobulin chain constant region; and B and D are linked to the second immunoglobulin chain constant region;
(c) A, B, C, and D are independently (i) absent; (ii) the first binder; (iii) the tumor targeting moiety, (iv) the cytokine molecule; (v) stromal modifying moiety; or (vi) the immune cell engager; and wherein at least one, two, or three of A, B, C, and D comprises the first binder.

In some embodiments, the second agent comprises the following configuration:

A-,B-[dimerization module]-C,-D,

wherein:
(a) the dimerization module comprises a first immunoglobulin chain constant region and a second immunoglobulin chain constant region;
(b) A and C are linked to the first immunoglobulin chain constant region; and B and D are linked to the second immunoglobulin chain constant region;
(c) A, B, C. and D are independently (i) absent; (ii) the second binder; (iii) the tumor targeting moiety, (iv) the cytokine molecule; (v) stromal modifying moiety; or (vi) the immune cell engager; and
wherein at least one, two, or three of A, B, C, and D comprises the second binder.

In some embodiments, the first immunoglobulin chain constant region comprises the first fragment crystallizable region (Fc region) and the second first immunoglobulin chain constant region comprises the second fragment crystallizable region (Fc region).

In some embodiments, dimerization of the first Fc region and the second Fc region is enhanced by providing an Fc interface of the first Fc region and the second Fc region with one or more of a paired cavity-protuberance, an electrostatic interaction, or a strand-exchange, such that a greater ratio of heteromultimer to homomultimer forms relative to a non-engineered interface.

In some embodiments, the immune cell engager is selected from the group consisting of a T cell engager, an NK cell engager, a B cell engager, a dendritic cell engager, a macrophage cell engager, and any combination thereof.

In some embodiments, the cytokine molecule is selected from the group consisting of interleukin-2 (IL-2) or functional variant thereof, interleukin-7 (IL-7) or functional variant thereof, interleukin-12 (IL-12) or functional variant thereof, interleukin-15 (IL-15) or functional variant thereof; interleukin-18 (IL-18) or functional variant thereof, inter-leukin-21 (IL-21) or functional variant thereof, interferon gamma or functional variant thereof, and any combination thereof.

In some embodiments, the cytokine molecule comprises interleukin-2 (IL-2) or functional variant thereof.

In some embodiments, the interleukin-2 (IL-2) or functional variant thereof comprises the sequence of SEQ ID NO: 2270 or SEQ ID NO: 2191.

In some embodiments, the cytokine molecule comprises interleukin-15 (IL-15) or functional variant thereof.

In some embodiments, the interleukin-15 (IL-15) or functional variant thereof comprises the sequence of SEQ ID NO: 2170.

In some embodiments, the cytokine molecule further comprises an IL15Ralpha dimerizing domain covalently linked the interleukin-15 (IL-15) or functional variant thereof.

In some embodiments, the IL15Ralpha dimerizing domain comprises an IL-15 receptor alpha sushi domain or functional variant thereof.

In some embodiments, the IL15Ralpha dimerizing domain comprises the sequence of SEQ ID NO: 3472.

In some embodiments, the interleukin-15 (IL-15) or functional variant thereof is covalently linked to the IL15Ralpha dimerizing domain via a linker.

In some embodiments, the interleukin-15 (IL-15) or functional variant thereof is covalently linked to the IL15Ralpha dimerizing domain via a linker comprising the sequence of SEQ ID NO: 3473.

In some embodiments, the cytokine molecule comprises the sequence of SEQ ID NO: 3474.

In some embodiments, the stromal modifying moiety is selected from the group consisting of a hyaluronidase, a collagenase, a chondroitinase, and a matrix metalloproteinase.

In some embodiments, the tumor-targeting moiety binds to a cancer antigen.

In some embodiments, wherein the tumor-targeting moiety binds to a cancer antigen.

In some embodiments, the cancer antigen is present on a hematological cancer, a solid tumor, a metastatic cancer, a soft tissue tumor, a metastatic lesion, or a combination thereof.

In some embodiments, the cancer antigen is a tumor antigen, a stromal antigen, or a hematological antigen.

In some embodiments, the cancer antigen is selected from the group consisting of BCMA, CD19, CD20, CD22, FcRH5, PDL1, CD47, ganglioside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, MC1R, β-catenin, BRCA1/2, CDK4, CML66, Fibronectin, p53, Ras, TGF-B receptor, AFP, ETA, MAGE, CA-125, BAGE, GAGE, CDC27, a actinin-4, TRP1/gp75, TRP2, gangliosides, WT1, Epidermal growth factor receptor (EGFR), MART-2, MUC1, MUC2, MUM1, MUM2, MUM3, NA88-1, NPM, OA1, OGT, RCC, RU11, RU12, SAGE, TRG, TSTA, Folate receptor alpha, L1-CAM, CAIX, gpA33, GD3, GM2, VEGFR, Integrins, Carbohydrates, IGF1R, TRAILR1, TRAILR2, RANKL, FAP, TGF-beta, hyaluronic acid, collagen, tenascin C, and tenascin W.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3445, wherein the VH is linked to a heavy chain constant region comprising the sequence of SEQ ID NO: 3649, wherein the heavy chain constant region comprises the first Fc region comprising the sequence of SEQ ID NO: 3447,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 3446, wherein the VL is linked to a light chain constant region comprising the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3448, wherein the VH is linked to a heavy chain constant region comprising the sequence of SEQ ID NO: 3649, wherein the heavy chain constant region comprises the first Fc region comprising the sequence of SEQ ID NO: 3447,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 3449, wherein the VL is linked to a light chain constant region comprising the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3450, wherein the VH is linked to a heavy chain constant region comprising the sequence of SEQ ID NO: 3649, wherein the heavy chain constant region comprises the first Fc region comprising the sequence of SEQ ID NO: 3447,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 3451, wherein the VL is linked to a light chain constant region comprising the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3450, wherein the VH is linked to a heavy chain constant region comprising the sequence of SEQ ID NO: 3649, wherein the heavy chain constant region comprises the first Fc region comprising the sequence of SEQ ID NO: 3447,
(ii) the second polypeptide chain comprises a cytokine molecule, wherein the cytokine molecule is linked to the second Fc region comprising the sequence of SEQ ID NO: 3454 or the sequence of SEQ ID NO: 3501 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 3451, wherein the VL is linked to a light chain constant region comprising the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 1346, wherein the VH is linked to a heavy chain constant region comprising the sequence of SEQ ID NO: 3452 or the sequence of SEQ ID NO: 3504, wherein the heavy chain constant region comprises the first Fc region comprising the sequence of SEQ ID NO: 3453 or the sequence of SEQ ID NO: 3505,
(ii) the second polypeptide chain comprises a cytokine molecule, wherein the cytokine molecule is linked to the second Fc region comprising the sequence of SEQ ID NO: 3454 or the sequence of SEQ ID NO: 3501 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 1349, wherein the VL is linked to a light chain constant region comprising the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 1196, wherein the VH is linked to a heavy chain constant region comprising the sequence of SEQ ID NO: 3649, wherein the heavy chain constant region comprises the first Fc region comprising the sequence of SEQ ID NO: 3447,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 1195, wherein the VL is linked to a light chain constant region comprising the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3455, wherein the VH is linked to a heavy chain constant region comprising the sequence of SEQ ID NO: 3649, wherein the heavy chain constant region comprises the first Fc region comprising the sequence of SEQ ID NO: 3447,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 3456, wherein the VL is linked to a light chain constant region comprising the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3457, wherein the VH is linked to a heavy chain constant region comprising the sequence of SEQ ID NO: 3459, wherein the heavy chain constant region comprises the first Fc region comprising the sequence of SEQ ID NO: 3460,
(ii) the second polypeptide chain comprises a cytokine molecule, wherein the cytokine molecule is linked to the second Fc region comprising the sequence of SEQ ID NO: 3462 via a linker comprising the sequence of SEQ ID NO: 3308, wherein the cytokine molecule comprises IL-2 or functional variant thereof comprising the sequence of SEQ ID NO: 2191, and
(iii) the third polypeptide chain comprises a VL comprising the sequence of SEQ ID NO: 3458, wherein the VL is linked to a light chain constant region comprising the sequence of SEQ ID NO: 3461.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3463, wherein the VH is linked to a heavy chain constant region comprising the sequence of SEQ ID NO: 3459, wherein the heavy chain constant region comprises the first Fc region comprising the sequence of SEQ ID NO: 3460,
(ii) the second polypeptide chain comprises a cytokine molecule, wherein the cytokine molecule is linked to the second Fc region comprising the sequence of SEQ ID NO: 3462 via a linker comprising the sequence of SEQ ID NO: 3308, wherein the cytokine molecule comprises IL-2 or functional variant thereof comprising the sequence of SEQ ID NO: 2191, and
(iii) the third polypeptide chain comprises a VL comprising the sequence of SEQ ID NO: 3464, wherein the VL is linked to a light chain constant region comprising the sequence of SEQ ID NO: 3461.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3465, wherein the VH is linked to a heavy chain constant region comprising the sequence of SEQ ID NO: 3467, wherein the heavy chain constant region comprises the first Fc region comprising the sequence of SEQ ID NO: 3468,
(ii) the second polypeptide chain comprises a cytokine molecule, wherein the cytokine molecule is linked to the second Fc region comprising the sequence of SEQ ID NO: 3469 via a linker comprising the sequence of SEQ ID NO: 3308, wherein the cytokine molecule comprises IL-2 or functional variant thereof comprising the sequence of SEQ ID NO: 2191, and
(iii) the third polypeptide chain comprises a VL comprising the sequence of SEQ ID NO: 3466, wherein the VL is linked to a light chain constant region comprising the sequence of SEQ ID NO: 3461.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3470, wherein the VH is linked to a heavy chain constant region comprising the sequence of SEQ ID NO: 3649, wherein the heavy chain constant region comprises the first Fc region comprising the sequence of SEQ ID NO: 3447,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 3471, wherein the VL is linked to a light chain constant region comprising the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 1346, wherein the VH is linked to a heavy chain constant region comprising the sequence of SEQ ID NO: 3452 or the sequence of SEQ ID NO: 3504, wherein the heavy chain constant region comprises the first Fc region comprising the sequence of SEQ ID NO: 3453 or the sequence of SEQ ID NO: 3505,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-15 or functional variant thereof comprising the sequence of SEQ ID NO: 2170, wherein the IL-15 or functional variant thereof is covalently linked to an IL15Ralpha dimerizing domain comprising the sequence of the SEQ ID NO: 3472 via a linker comprising the sequence of SEQ ID NO: 3473, and
(iii) the third polypeptide chain comprises a VL comprising the sequence of SEQ ID NO: 1349, wherein the VL is linked to a light chain constant region comprising the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 1346, wherein the VH is linked to a heavy chain constant region comprising the sequence of SEQ ID NO: 3452 or the sequence of SEQ ID NO: 3504, wherein the heavy chain constant region comprises the first Fc region comprising the sequence of SEQ ID NO: 3453 or the sequence of SEQ ID NO: 3505,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-15 or functional variant thereof comprising the sequence of SEQ ID NO: 2170, wherein the IL-15 or functional variant thereof is covalently linked to an IL15Ralpha dimerizing domain comprising the sequence of the SEQ ID NO: 3472 via a linker comprising the sequence of SEQ ID NO: 3473, and
(iii) the third polypeptide chain comprises a VL comprising the sequence of SEQ ID NO: 3475, wherein the VL is linked to a light chain constant region comprising the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 1346, wherein the VH is linked to a heavy chain constant region comprising the sequence of SEQ ID NO: 3452 or the sequence of SEQ ID NO: 3504, wherein the heavy chain constant region comprises the first Fc region comprising the sequence of SEQ ID NO: 3453 or the sequence of SEQ ID NO: 3505,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-15 or functional variant thereof comprising the sequence of SEQ ID NO: 2170, wherein the IL-15 or functional variant thereof is covalently linked to an IL15Ralpha dimerizing domain comprising the sequence of the SEQ ID NO: 3472 via a linker comprising the sequence of SEQ ID NO: 3473, and
(iii) the third polypeptide chain comprises a VL comprising the sequence of SEQ ID NO: 3476, wherein the VL is linked to a light chain constant region comprising the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3445, wherein the VH is linked to a heavy chain constant region comprising the sequence of SEQ ID NO: 3649, wherein the heavy chain constant region comprises the first Fc region comprising the sequence of SEQ ID NO: 3447,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 3446, wherein the VL is linked to a light chain constant region comprising the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3448, wherein the VH is linked to a heavy chain constant region comprising the sequence of SEQ ID NO: 3649, wherein the heavy chain constant region comprises the first Fc region comprising the sequence of SEQ ID NO: 3447,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 3449, wherein the VL is linked to a light chain constant region comprising the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3450, wherein the VH is linked to a heavy chain constant region comprising the sequence of SEQ ID NO: 3649, wherein the heavy chain constant region comprises the first Fc region comprising the sequence of SEQ ID NO: 3447,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 3451, wherein the VL is linked to a light chain constant region comprising the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3450, wherein the VH is linked to a heavy chain constant region comprising the sequence of SEQ ID NO: 3649, wherein the heavy chain constant region comprises the first Fc region comprising the sequence of SEQ ID NO: 3447,
(ii) the second polypeptide chain comprises a cytokine molecule, wherein the cytokine molecule is linked to the second Fc region comprising the sequence of SEQ ID NO: 3454 or the sequence of SEQ ID NO: 3501 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 3451, wherein the VL is linked to a light chain constant region comprising the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 1346, wherein the VH is linked to a heavy chain constant region comprising the sequence of SEQ ID NO: 3452 or the sequence of SEQ ID NO: 3504, wherein the heavy chain constant region comprises the first Fc region comprising the sequence of SEQ ID NO: 3453 or the sequence of SEQ ID NO: 3505,
(ii) the second polypeptide chain comprises a cytokine molecule, wherein the cytokine molecule is linked to the second Fc region comprising the sequence of SEQ ID NO: 3454 or the sequence of SEQ ID NO: 3501 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 1349, wherein the VL is linked to a light chain constant region comprising the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 1196, wherein the VH is linked to a heavy chain constant region comprising the sequence of SEQ ID NO: 3649, wherein the heavy chain constant region comprises the first Fc region comprising the sequence of SEQ ID NO: 3447,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 1195, wherein the VL is linked to a light chain constant region comprising the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3455, wherein the VH is linked to a heavy chain constant region comprising the sequence of SEQ ID NO: 3649, wherein the heavy chain constant region comprises the first Fc region comprising the sequence of SEQ ID NO: 3447,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 3456, wherein the VL is linked to a light chain constant region comprising the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3457, wherein the VH is linked to a heavy chain constant region comprising the sequence of SEQ ID NO: 3459, wherein the heavy chain constant region comprises the first Fc region comprising the sequence of SEQ ID NO: 3460,
(ii) the second polypeptide chain comprises a cytokine molecule, wherein the cytokine molecule is linked to the second Fc region comprising the sequence of SEQ ID NO: 3462 via a linker comprising the sequence of SEQ ID NO: 3308, wherein the cytokine molecule comprises IL-2 or functional variant thereof comprising the sequence of SEQ ID NO: 2191, and
(iii) the third polypeptide chain comprises a VL comprising the sequence of SEQ ID NO: 3458, wherein the VL is linked to a light chain constant region comprising the sequence of SEQ ID NO: 3461.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3463, wherein the VH is linked to a heavy chain constant region comprising the sequence of SEQ ID NO: 3459, wherein the heavy chain constant region comprises the first Fc region comprising the sequence of SEQ ID NO: 3460,
(ii) the second polypeptide chain comprises a cytokine molecule, wherein the cytokine molecule is linked to the second Fc region comprising the sequence of SEQ ID NO: 3462 via a linker comprising the sequence of SEQ ID NO: 3308, wherein the cytokine molecule comprises IL-2 or functional variant thereof comprising the sequence of SEQ ID NO: 2191, and
(iii) the third polypeptide chain comprises a VL comprising the sequence of SEQ ID NO: 3464, wherein the VL is linked to a light chain constant region comprising the sequence of SEQ ID NO: 3461.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3465, wherein the VH is linked to a heavy chain constant region comprising the sequence of SEQ ID NO: 3467, wherein the heavy chain constant region comprises the first Fc region comprising the sequence of SEQ ID NO: 3468,
(ii) the second polypeptide chain comprises a cytokine molecule, wherein the cytokine molecule is linked to the second Fc region comprising the sequence of SEQ ID NO: 3469 via a linker comprising the sequence of SEQ ID NO: 3308, wherein the cytokine molecule comprises IL-2 or functional variant thereof comprising the sequence of SEQ ID NO: 2191, and
(iii) the third polypeptide chain comprises a VL comprising the sequence of SEQ ID NO: 3466, wherein the VL is linked to a light chain constant region comprising the sequence of SEQ ID NO: 3461.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3470, wherein the VH is linked to a heavy chain constant region comprising the sequence of SEQ ID NO: 3649, wherein the heavy chain constant region comprises the first Fc region comprising the sequence of SEQ ID NO: 3447,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 3471, wherein the VL is linked to a light chain constant region comprising the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 1346, wherein the VH is linked to a heavy chain constant region comprising the sequence of SEQ ID NO: 3452 or the sequence of SEQ ID NO: 3504, wherein the heavy chain constant region comprises the first Fc region comprising the sequence of SEQ ID NO: 3453 or the sequence of SEQ ID NO: 3505,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-15 or functional variant thereof comprising the sequence of SEQ ID NO: 2170, wherein the IL-15 or functional variant thereof is covalently linked to an IL15Ralpha dimerizing domain comprising the sequence of the SEQ ID NO: 3472 via a linker comprising the sequence of SEQ ID NO: 3473, and
(iii) the third polypeptide chain comprises a VL comprising the sequence of SEQ ID NO: 1349, wherein the VL is linked to a light chain constant region comprising the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 1346, wherein the VH is linked to a heavy chain constant region comprising the sequence of SEQ ID NO: 3452 or the sequence of SEQ ID NO: 3504, wherein the heavy chain constant region comprises the first Fc region comprising the sequence of SEQ ID NO: 3453 or the sequence of SEQ ID NO: 3505,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-15 or functional variant thereof comprising the sequence of SEQ ID NO: 2170, wherein the IL-15 or functional variant thereof is covalently linked to an IL15Ralpha dimerizing domain comprising the sequence of the SEQ ID NO: 3472 via a linker comprising the sequence of SEQ ID NO: 3473, and
(iii) the third polypeptide chain comprises a VL comprising the sequence of SEQ ID NO: 3475, wherein the VL is linked to a light chain constant region comprising the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 1346, wherein the VH is linked to a heavy chain constant region comprising the sequence of SEQ ID NO: 3452 or the sequence of SEQ ID NO: 3504, wherein the heavy chain constant region comprises the first Fc region comprising the sequence of SEQ ID NO: 3453 or the sequence of SEQ ID NO: 3505,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-15 or functional variant thereof comprising the sequence of SEQ ID NO: 2170, wherein the IL-15 or functional variant thereof is covalently linked to an IL15Ralpha dimerizing domain comprising the sequence of the SEQ ID NO: 3472 via a linker comprising the sequence of SEQ ID NO: 3473, and
(iii) the third polypeptide chain comprises a VL comprising the sequence of SEQ ID NO: 3476, wherein the VL is linked to a light chain constant region comprising the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3338;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3339.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3318;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3319.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3328;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3329.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3328;
(ii) the second polypeptide chain comprises a sequence having at least 75% sequence identity to the sequence of SEQ ID NO: 3350 or the sequence of SEQ ID NO: 3500; and
(iii) the third polypeptide chain comprises a sequence having at least 75% sequence identity to the sequence of SEQ ID NO: 3329.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3348 or the sequence of SEQ ID NO: 3502;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3349.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3358;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3359.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3368;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3369.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3378;
(ii) the second polypeptide chain comprises a sequence having at least 75% sequence identity to the sequence of SEQ ID NO: 3380; and
(iii) the third polypeptide chain comprises a sequence having at least 75% sequence identity to the sequence of SEQ ID NO: 3379.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3388;
(ii) the second polypeptide chain comprises a sequence having at least 75% sequence identity to the sequence of SEQ ID NO: 3390; and
(iii) the third polypeptide chain comprises a sequence having at least 75% sequence identity to the sequence of SEQ ID NO: 3389.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3398;
(ii) the second polypeptide chain comprises a sequence having at least 75% sequence identity to the sequence of SEQ ID NO: 3400; and
(iii) the third polypeptide chain comprises a sequence having at least 75% sequence identity to the sequence of SEQ ID NO: 3399.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3408;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3409.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3348 or the sequence of SEQ ID NO: 3502;
(ii) the second polypeptide chain comprises a sequence having at least 75% sequence identity to the sequence of SEQ ID NO: 3420; and
(iii) the third polypeptide chain comprises a sequence having at least 75% sequence identity to the sequence of SEQ ID NO: 3349.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3348 or the sequence of SEQ ID NO: 3502;
(ii) the second polypeptide chain comprises a sequence having at least 75% sequence identity to the sequence of SEQ ID NO: 3420; and
(iii) the third polypeptide chain comprises a sequence having at least 75% sequence identity to the sequence of SEQ ID NO: 3428.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3348 or the sequence of SEQ ID NO: 3502;
(ii) the second polypeptide chain comprises a sequence having at least 75% sequence identity to the sequence of SEQ ID NO: 3420; and
(iii) the third polypeptide chain comprises a sequence having at least 75% sequence identity to the sequence of SEQ ID NO: 3437.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3338;
(ii) the second polypeptide chain comprises the sequence of SEQ ID NO: 3340 or the sequence of SEQ ID NO: 3503; and
(iii) the third polypeptide chain comprises the sequence of SEQ ID NO: 3339.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3318;
(ii) the second polypeptide chain comprises the sequence of SEQ ID NO: 3340 or the sequence of SEQ ID NO: 3503; and
(iii) the third polypeptide chain comprises the sequence of SEQ ID NO: 3319.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3328;
(ii) the second polypeptide chain comprises the sequence of SEQ ID NO: 3340 or the sequence of SEQ ID NO: 3503; and
(iii) the third polypeptide chain comprises the sequence of SEQ ID NO: 3329.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3328;
(ii) the second polypeptide chain comprises the sequence of SEQ ID NO: 3350 or the sequence of SEQ ID NO: 3500; and
(iii) the third polypeptide chain comprises the sequence of SEQ ID NO: 3329.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3348 or the sequence of SEQ ID NO: 3502;
(ii) the second polypeptide chain comprises the sequence of SEQ ID NO: 3340 or the sequence of SEQ ID NO: 3503; and
(iii) the third polypeptide chain comprises the sequence of SEQ ID NO: 3349.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3358;
(ii) the second polypeptide chain comprises the sequence of SEQ ID NO: 3340 or the sequence of SEQ ID NO: 3503; and
(iii) the third polypeptide chain comprises the sequence of SEQ ID NO: 3359.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3368;
(ii) the second polypeptide chain comprises the sequence of SEQ ID NO: 3340 or the sequence of SEQ ID NO: 3503; and
(iii) the third polypeptide chain comprises the sequence of SEQ ID NO: 3369.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3378;
(ii) the second polypeptide chain comprises the sequence of SEQ ID NO: 3380; and
(iii) the third polypeptide chain comprises the sequence of SEQ ID NO: 3379.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3388;
(ii) the second polypeptide chain comprises the sequence of SEQ ID NO: 3390; and
(iii) the third polypeptide chain comprises the sequence of SEQ ID NO: 3389.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3398;
(ii) the second polypeptide chain comprises the sequence of SEQ ID NO: 3400; and
(iii) the third polypeptide chain comprises the sequence of SEQ ID NO: 3399.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3408;
(ii) the second polypeptide chain comprises the sequence of SEQ ID NO: 3340 or the sequence of SEQ ID NO: 3503; and
(iii) the third polypeptide chain comprises the sequence of SEQ ID NO: 3409.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3348 or the sequence of SEQ ID NO: 3502;
(ii) the second polypeptide chain comprises the sequence of SEQ ID NO: 3420; and
(iii) the third polypeptide chain comprises the sequence of SEQ ID NO: 3349.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3348 or the sequence of SEQ ID NO: 3502;
(ii) the second polypeptide chain comprises the sequence of SEQ ID NO: 3420; and
(iii) the third polypeptide chain comprises the sequence of SEQ ID NO: 3428.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3348 or the sequence of SEQ ID NO: 3502;
(ii) the second polypeptide chain comprises the sequence of SEQ ID NO: 3420; and
(iii) the third polypeptide chain comprises the sequence of SEQ ID NO: 3437.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3338;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3339.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3318;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3319.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3328;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3329.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3328;
(ii) the second polypeptide chain comprises a sequence having at least 75% sequence identity to the sequence of SEQ ID NO: 3350 or the sequence of SEQ ID NO: 3500; and
(iii) the third polypeptide chain comprises a sequence having at least 75% sequence identity to the sequence of SEQ ID NO: 3329.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3348 or the sequence of SEQ ID NO: 3502;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3349.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3358;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3359.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3368;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3369.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3378;
(ii) the second polypeptide chain comprises a sequence having at least 75% sequence identity to the sequence of SEQ ID NO: 3380; and
(iii) the third polypeptide chain comprises a sequence having at least 75% sequence identity to the sequence of SEQ ID NO: 3379.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3388;
(ii) the second polypeptide chain comprises a sequence having at least 75% sequence identity to the sequence of SEQ ID NO: 3390; and
(iii) the third polypeptide chain comprises a sequence having at least 75% sequence identity to the sequence of SEQ ID NO: 3389.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3398;
(ii) the second polypeptide chain comprises a sequence having at least 75% sequence identity to the sequence of SEQ ID NO: 3400; and
(iii) the third polypeptide chain comprises a sequence having at least 75% sequence identity to the sequence of SEQ ID NO: 3399.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3408;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3409.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3348 or the sequence of SEQ ID NO: 3502;
(ii) the second polypeptide chain comprises a sequence having at least 75% sequence identity to the sequence of SEQ ID NO: 3420; and
(iii) the third polypeptide chain comprises a sequence having at least 75% sequence identity to the sequence of SEQ ID NO: 3349.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3348 or the sequence of SEQ ID NO: 3502;
(ii) the second polypeptide chain comprises a sequence having at least 75% sequence identity to the sequence of SEQ ID NO: 3420; and
(iii) the third polypeptide chain comprises a sequence having at least 75% sequence identity to the sequence of SEQ ID NO: 3428.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3348 or the sequence of SEQ ID NO: 3502;
(ii) the second polypeptide chain comprises a sequence having at least 75% sequence identity to the sequence of SEQ ID NO: 3420; and
(iii) the third polypeptide chain comprises a sequence having at least 75% sequence identity to the sequence of SEQ ID NO: 3437.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3338;
(ii) the second polypeptide chain comprises the sequence of SEQ ID NO: 3340 or the sequence of SEQ ID NO: 3503; and
(iii) the third polypeptide chain comprises the sequence of SEQ ID NO: 3339.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3318;
(ii) the second polypeptide chain comprises the sequence of SEQ ID NO: 3340 or the sequence of SEQ ID NO: 3503; and
(iii) the third polypeptide chain comprises the sequence of SEQ ID NO: 3319.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3328.
(ii) the second polypeptide chain comprises the sequence of SEQ ID NO: 3340 or the sequence of SEQ ID NO: 3503; and
(iii) the third polypeptide chain comprises the sequence of SEQ ID NO: 3329.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3328;
(ii) the second polypeptide chain comprises the sequence of SEQ ID NO: 3350 or the sequence of SEQ ID NO: 3500; and
(iii) the third polypeptide chain comprises the sequence of SEQ ID NO: 3329.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3348 or the sequence of SEQ ID NO: 3502;
(ii) the second polypeptide chain comprises the sequence of SEQ ID NO: 3340 or the sequence of SEQ ID NO: 3503; and
(iii) the third polypeptide chain comprises the sequence of SEQ ID NO: 3349.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3358;
(ii) the second polypeptide chain comprises the sequence of SEQ ID NO: 3340 or the sequence of SEQ ID NO: 3503; and
(iii) the third polypeptide chain comprises the sequence of SEQ ID NO: 3359.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3368;
(ii) the second polypeptide chain comprises the sequence of SEQ ID NO: 3340 or the sequence of SEQ ID NO: 3503; and
(iii) the third polypeptide chain comprises the sequence of SEQ ID NO: 3369.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3378;
(ii) the second polypeptide chain comprises the sequence of SEQ ID NO: 3380; and
(iii) the third polypeptide chain comprises the sequence of SEQ ID NO: 3379.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3388.
(ii) the second polypeptide chain comprises the sequence of SEQ ID NO: 3390; and
(iii) the third polypeptide chain comprises the sequence of SEQ ID NO: 3389.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3398;
(ii) the second polypeptide chain comprises the sequence of SEQ ID NO: 3400; and
(iii) the third polypeptide chain comprises the sequence of SEQ ID NO: 3399.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3408;
(ii) the second polypeptide chain comprises the sequence of SEQ ID NO: 3340 or the sequence of SEQ ID NO: 3503; and
(iii) the third polypeptide chain comprises the sequence of SEQ ID NO: 3409.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3348 or the sequence of SEQ ID NO: 3502;
(ii) the second polypeptide chain comprises the sequence of SEQ ID NO: 3420; and
(iii) the third polypeptide chain comprises the sequence of SEQ ID NO: 3349.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3348 or the sequence of SEQ ID NO: 3502;
(ii) the second polypeptide chain comprises the sequence of SEQ ID NO: 3420; and
(iii) the third polypeptide chain comprises the sequence of SEQ ID NO: 3428.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3348 or the sequence of SEQ ID NO: 3502;
(ii) the second polypeptide chain comprises the sequence of SEQ ID NO: 3420; and
(iii) the third polypeptide chain comprises the sequence of SEQ ID NO: 3437.

In some embodiments, the disease or condition is cancer.

In some embodiments, the cancer is a solid tumor.

In some embodiments, the solid tumor is melanoma, pancreatic cancer, breast cancer, colorectal cancer, lung cancer, skin cancer, ovarian cancer, or liver cancer.

In some embodiments, the solid tumor is selected from the group consisting of high mutational burden (TMB-H), microsatellite instability/DNA mismatch repair (MSI-H/dMMR), virally associated tumor, metastatic triple-negative breast cancer (mTNBC), re-lapsed and refractory epithelial ovarian cancer, metastatic castration-resistant prostate cancer (mCRPC), K-Ras wild type CRC, K-Ras mutant CRC, and primary stage IV or recurrent non-small cell lung cancer (NSCLC).

In some embodiments, the virally associated tumor comprises Merkel cell carcinoma, cervical cancer, oropharyngeal cancer, anal cancer, penile cancer, vaginal cancer, or vulvar cancer.

In some embodiments, the cancer is a hematological cancer.

In some embodiments, the hematological cancer is a B-cell or T cell malignancy.

In some embodiments, the B-cell or T cell malignancy is Hodgkin's lymphoma, Non-Hodgkin's lymphoma, acute myeloid leukemia (AML), chronic myeloid leukemia, myelodysplastic syndrome, multiple myeloma, or acute lymphocytic leukemia.

In some embodiments, the Non-Hodgkin's lymphoma is B cell lymphoma, diffuse large B cell lymphoma, follicular lymphoma, chronic lymphocytic leukemia, mantle cell lymphoma, marginal zone B-cell lymphoma, Burkitt lymphoma, lymphoplasmacytic lymphoma, or hairy cell leukemia.

In some embodiments, the administering comprises administering a first pharmaceutical composition comprising the first agent, and a second pharmaceutical com-position comprising the second agent, wherein the first pharmaceutical composition and the second pharmaceutical composition independently further comprises a pharmaceutically acceptable excipient, carrier or diluent.

In another aspect, provided herein is, inter alia, a combination therapy for treating a disease or condition in a subject in need thereof comprising administering a first agent that comprises a first binder that binds to a first target molecule to the subject, and administering a second agent that comprises a second binder that binds to a second target molecule to the subject, thereby treating the disease or condition in the subject:
wherein an administration of the first agent is followed by an administration of the second agent;
wherein the first target molecule and the second target molecule are independently selected from the group consisting of a T cell receptor alpha variable region (TCRαV) and a T cell receptor beta variable region (TCRβV); and
wherein the first target molecule and the second target molecule are different.

In some embodiments, the first agent and the second agent are not concomitantly administered to the subject.

In some embodiments, administering the first agent comprises administering a therapeutically effective amount of the first agent to the subject.

In some embodiments, administering the second agent comprises administering a therapeutically effective amount of the second agent to the subject.

In some embodiments, the combination therapy is more effective to treat the disease or condition in the subject relative to a method in which the first agent is administered to the subject but not the second agent.

In some embodiments, the combination therapy is more effective to treat the disease or condition in the subject relative to a method in which the second agent is administered to the subject but not the first agent.

In some embodiments, the combination therapy is more effective to treat the disease or condition in the subject relative to a method in which the first agent is administered to the subject at a first administration and the first agent is administered to the subject subsequently at a second administration.

In some embodiments, the combination therapy is more effective to treat the disease or condition in the subject relative to a method in which the second agent is administered to the subject at a first administration and the second agent is administered to the subject subsequently at a second administration.

In some embodiments, the combination therapy is more effective to treat the disease or condition in the subject relative to a method in which the second agent is administered to the subject at a first administration and the first agent is administered to the subject subsequently at a second administration.

In some embodiments, the administration of the first agent comprises one or more doses.

In some embodiments, the administration of the first agent comprises two or more doses.

In some embodiments, the administration of the first agent comprises one dose per week.

In some embodiments, the administration of the first agent comprises two or more doses per week.

In some embodiments, the first agent is administered to the subject once every two weeks.

In some embodiments, the first agent is administered to the subject once every three weeks.

In some embodiments, the first agent is administered to the subject once every week for at least 1, 2, 3, or 4 weeks, at least 1, 2, 3, 4, 5, 6, 7, 8, 9, 10, 11 or 12 months, or at least 1, 2, or 3 years.

In some embodiments, the administration of the first agent comprises two or more doses per week for at least 1, 2, 3, or 4 weeks, at least 1, 2, 3, 4, 5, 6, 7, 8, 9, 10, 11 or 12 months, or at least 1, 2, or 3 years.

In some embodiments, the first agent is administered to the subject once every two weeks for at least 1, 2, 3, or 4 weeks, at least 1, 2, 3, 4, 5, 6, 7, 8, 9, 10, 11 or 12 months, or at least 1, 2, or 3 years.

In some embodiments, the first agent is administered to the subject once every three weeks for at least 1, 2, 3, or 4 weeks, at least 1, 2, 3, 4, 5, 6, 7, 8, 9, 10, 11 or 12 months, or at least 1, 2, or 3 years.

In some embodiments, the administration of the second agent comprises one or more doses.

In some embodiments, the administration of the second agent comprises two or more doses.

In some embodiments, the administration of the second agent comprises one dose per week.

In some embodiments, the administration of the second agent comprises two or more doses per week.

In some embodiments, the second agent is administered to the subject once every two weeks.

In some embodiments, the second agent is administered to the subject once every three weeks.

In some embodiments, the second agent is administered to the subject once every week for at least 1, 2, 3, or 4 weeks, at least 1, 2, 3, 4, 5, 6, 7, 8, 9, 10, 11 or 12 months, or at least 1, 2, or 3 years.

In some embodiments, the administration of the second agent comprises two or more doses per week for at least 1, 2, 3, or 4 weeks, at least 1, 2, 3, 4, 5, 6, 7, 8, 9, 10, 11 or 12 months, or at least 1, 2, or 3 years.

In some embodiments, the second agent is administered to the subject once every two weeks for at least 1, 2, 3, or 4 weeks, at least 1, 2, 3, 4, 5, 6, 7, 8, 9, 10, 11 or 12 months, or at least 1, 2, or 3 years.

In some embodiments, the second agent is administered to the subject once every three weeks for at least 1, 2, 3, or 4 weeks, at least 1, 2, 3, 4, 5, 6, 7, 8, 9, 10, 11 or 12 months, or at least 1, 2, or 3 years.

In some embodiments, the administration of the first agent and the administration of the second agent is separated by one week.

In some embodiments, the administration of the first agent and the administration of the second agent is separated by two or more weeks.

In some embodiments, the administration of the first agent and the administration of the second agent is separated by 1 day, 2 days, 3 days, 4 days, 5 days, 6 days, 1 week, 2 weeks, 3 weeks, 4 weeks, 1 month, 2 months, 3 months, 4 months, 5 months, 6 months, 7 months, 8 months, 9 months, 10 months, 11 months, or 12 months.

In some embodiments, the administration of the first agent and the second agent is repeated twice or more.

In some embodiments, the first agent and the second agent are administered to the subject according to a dosing regimen, wherein the dosing regimen comprises an administration of the first agent once every week for two weeks followed by an administration of the second agent once a week for two weeks, wherein the second agent is administered to the subject one week after the last administration of the first agent.

In some embodiments, the dosing regimen is repeated twice or more.

In some embodiments, the dosing regimen is repeated at the interval of one week.

In some embodiments, the dosing regimen is repeated at the interval of 1 day, 2 days, 3 days, 4 days, 5 days, 6 days, 1 week, 2 weeks, 3 weeks, 4 weeks, 1 month, 2 months, 3 months, 4 months, 5 months, 6 months, 7 months, 8 months, 9 months, 10 months, 11 months, or 12 months.

In some embodiments, the first agent and the second agent are administered to the subject in a same amount.

In some embodiments, the first agent and the second agent are administered to the subject in different amounts.

In some embodiments, the administration of the first agent comprises two or more doses, and each dose comprises a same amount of the first agent.

In some embodiments, the administration of the first agent comprises two or more doses, and each dose comprises different amounts of the first agent.

In some embodiments, the administration of the second agent comprises two or more doses, and each dose comprises a same amount of the second agent.

In some embodiments, the administration of the second agent comprises two or more doses, and each dose comprises different amounts of the second agent.

In some embodiments, the first agent is administered to the subject at a dose of from about 0.001 mg/kg to about 10 mg/kg.

In some embodiments, the second agent is administered to the subject at a dose of from about 0.001 mg/kg to about 10 mg/kg.

In some embodiments, the first agent is administered to the subject at a dose of about 0.3 mg/kg or about 1 mg/kg.

In some embodiments, the second agent is administered to the subject at a dose of about 0.3 mg/kg or about 1 mg/kg.

In some embodiments, the first target molecule is a first TCRβV, and the second target molecule is a second TCRβV, wherein the first TCRβV and the second TCRβV are different.

In some embodiments, the first target molecule is a first TCRαV, and the second target molecule is a second TCRαV, wherein the first TCRαV and the second TCRαV are different.

In some embodiments, the first target molecule is a TCRβV, and the second target molecule is a TCRαV.

In some embodiments, the first target molecule is a TCRαV, and the second target molecule is a TCRβV.

In some embodiments, the first target molecule is human TCRβV6.

In some embodiments, the first target molecule is human TCRβV10.

In some embodiments, the second target molecule is human TCRβV15.

In some embodiments, the second target molecule is human TCRβV27.

In some embodiments, the second target molecule is human TCRβV5.

In some embodiments, the first target molecule is human TCRβV6, and the second target molecule is human TCRβV5.

In some embodiments, the first target molecule is human TCRβV6, and the second target molecule is human TCRβV15.

In some embodiments, the first target molecule is human TCRβV6, and the second target molecule is human TCRβV27.

In some embodiments, the first target molecule is human TCRβV10, and the second target molecule is human TCRβV5.

In some embodiments, the first target molecule is human TCRβV10, and the second target molecule is human TCRβV15.

In some embodiments, the first target molecule is human TCRβV10, and the second target molecule is human TCRβV27.

In some embodiments, the first target molecule is human TRBV5, human TRBV6, human TRBV10, human TRBV7, human TRBV19, human TRBV20, or human TRBV28.

In some embodiments, the first target molecule is human TRBV5-1, human TRBV6-5, human TRBV10-1, human TRBV10-2, human TRBV10-3, human TRBV7-2, human TRBV7-3, human TRBV19, human TRBV20-1, or human TRBV28.

In some embodiments, the first target molecule is human TRAV12, human TRAV13, human TRAV19, human TRAV21, or human TRAV30.

In some embodiments, the first target molecule is human TRAV12-2, human TRAV13-1, human TRAV19, human TRAV21, or human TRAV30.

In some embodiments, the second target molecule is human TRBV5, human TRBV15, human TRBV27, human TRBV6, human TRBV10, human TRBV7, human TRBV19, human TRBV20, or human TRBV28.

In some embodiments, the second target molecule is human TRBV5-1, human TRBV6-5, human TRBV10-1, human TRBV10-2, human TRBV10-3, human TRBV7-2, human TRBV7-3, human TRBV15, human TRBV27, human TRBV19, human TRBV20-1, or human TRBV28.

In some embodiments, the second target molecule is human TRAV12, human TRAV13, human TRAV19, human TRAV21, or human TRAV30.

In some embodiments, the second target molecule is human TRAV12-2, human TRAV13-1, human TRAV19, human TRAV21, or human TRAV30.

In some embodiments, the first target molecule is human TRBV5, human TRBV6, human TRBV10, human TRBV7, human TRBV19, human TRBV20, or human TRBV28, and the second target molecule is human TRBV5, human TRBV6, human TRBV10, human TRBV7, human TRBV15, human TRBV27, human TRBV19, human TRBV20, or human TRBV28, wherein the first target molecule and the second target molecule are different.

In some embodiments, the first target molecule and the second target molecule are:
human TRBV5 and human TRBV5, respectively; human TRBV5 and human TRBV6, respectively; human TRBV5 and human TRBV10, respectively; human TRBV5 and human TRBV7, respectively; human TRBV5 and human TRBV19, respectively; human TRBV5 and human TRBV20, respectively; human TRBV5 and human TRBV28, respectively; human TRBV6 and human TRBV5, respectively; human TRBV6 and human TRBV6, respectively; human TRBV6 and human TRBV10, respectively; human TRBV6 and human TRBV7, respectively; human TRBV6 and human TRBV15, respectively; human TRBV6 and human TRBV27, respectively; human TRBV6 and human TRBV19, respectively; human TRBV6 and human TRBV20, respectively; human TRBV6 and human TRBV28, respectively; human TRBV10 and human TRBV5, respectively; human TRBV10 and human TRBV6, respectively; human TRBV10 and human TRBV10, respectively; human TRBV10 and human TRBV7, respectively; human TRBV10 and human TRBV15, respectively; human TRBV10 and human TRBV27, respectively; human TRBV10 and human TRBV19, respectively; human TRBV10 and human TRBV20, respectively; human TRBV10 and human TRBV28, respectively; human TRBV7 and human TRBV5, respectively; human TRBV7 and human TRBV6, respectively; human TRBV7 and human TRBV10, respectively; human TRBV7 and human TRBV7, respectively; human TRBV7 and human TRBV19, respectively; human TRBV7 and human TRBV20, respectively; human TRBV7 and human TRBV28, respectively; human TRBV19 and human TRBV5, respectively; human TRBV19 and human TRBV6, respectively; human TRBV19 and human TRBV10, respectively; human TRBV19 and human TRBV7, respectively; human TRBV19 and human TRBV19, respectively; human TRBV19 and human TRBV20, respectively; human TRBV19 and human TRBV28, respectively; human TRBV20 and human TRBV5, respectively; human TRBV20 and human TRBV6, respectively; human TRBV20 and human TRBV10, respectively, human TRBV20 and human TRBV7, respectively; human TRBV20 and human TRBV19, respectively; human TRBV20 and human TRBV20, respectively; human TRBV20 and human TRBV28, respectively; human TRBV28 and human TRBV5, respectively; human TRBV28 and human TRBV6, respectively; human TRBV28 and human TRBV10, respectively; human TRBV28 and human TRBV7, respectively; human TRBV28 and human TRBV19, respectively; human TRBV28 and human TRBV20, respectively; or human TRBV28 and human TRBV28, respectively.

In some embodiments, the first target molecule is human TRBV5-1, human TRBV6-5, human TRBV10-1, human TRBV10-2, human TRBV10-3, human TRBV7-2, human TRBV7-3, human TRBV19, human TRBV20-1, or human TRBV28, and the second target molecule is human TRBV5-1, human TRBV6-5, human TRBV10-1, human TRBV10-2, human TRBV10-3, human TRBV7-2, human TRBV7-3, human TRBV15, human TRBV27, human TRBV19, human TRBV20-1, or human TRBV28, wherein the first target molecule and the second target molecule are different.

In some embodiments, the first target molecule and the second target molecule are:
Human TRBV5-1 and human TRBV6-5, respectively; human TRBV5-1 and human TRBV10-1, respectively; human TRBV5-1 and human TRBV10-2, respectively; human TRBV5-1 and human TRBV10-3, respectively; human TRBV5-1 and human TRBV7-2, respectively; human TRBV5-1 and human TRBV7-3, respectively; human TRBV5-1 and human TRBV19, respectively; human TRBV5-1 and human TRBV20-1, respectively; human TRBV5-1 and human TRBV28, respectively; human TRBV6-5 and human TRBV5-1, respectively; human TRBV6-5 and human TRBV7-2, respectively; human TRBV6-5 and human TRBV7-3, respectively; human TRBV6-5 and human TRBV15, respectively; human TRBV6-5 and human TRBV27, respectively; human TRBV6-5 and human TRBV19, respectively; human TRBV6-5 and human TRBV20-1, respectively; human TRBV6-5 and human TRBV28, respectively; human TRBV10-1 and human TRBV5-1, respectively; human TRBV10-1 and human TRBV7-2, respectively; human TRBV10-1 and human TRBV7-3, respectively; human TRBV10-1 and human TRBV15, respectively; human TRBV10-1 and human TRBV27, respectively; human TRBV10-1 and human TRBV19, respectively; human TRBV10-1 and human TRBV20-1, respectively; human TRBV10-1 and human TRBV28, respectively; human TRBV10-2 and human TRBV5-1, respectively, human TRBV10-2 and human TRBV7-2, respectively; human TRBV10-2 and human TRBV7-3, respectively; human TRBV10-2 and human TRBV15, respectively; human TRBV10-2 and human TRBV27, respectively; human TRBV10-2 and human TRBV19, respectively; human TRBV10-2 and human TRBV20-1, respectively; human TRBV10-2 and human TRBV28, respectively; human TRBV10-3 and human TRBV5-1, respectively; human TRBV10-3 and human TRBV7-2, respectively; human TRBV10-3 and human TRBV7-3, respectively; human TRBV10-3 and human TRBV15, respectively; human TRBV10-3 and human TRBV27, respectively; human TRBV10-3 and human TRBV19, respectively; human TRBV10-3 and human TRBV20-1, respectively; human TRBV10-3 and human TRBV28, respectively; human TRBV7-2 and human TRBV5-1, respectively; human TRBV7-2 and human TRBV6-5, respectively; human TRBV7-2 and human TRBV10-1, respectively; human TRBV7-2 and human TRBV10-2, respectively; human TRBV7-2 and human TRBV10-3, respectively; human TRBV7-2 and human TRBV7-3, respectively; human TRBV7-2 and human TRBV19, respectively; human TRBV7-2 and human TRBV20-1, respectively; human TRBV7-2 and human TRBV28, respectively; human TRBV7-3 and human TRBV5-1, respectively; human TRBV7-3 and human TRBV6-5, respectively; human TRBV7-3 and human TRBV10-1, respectively; human TRBV7-3 and human TRBV10-2, respectively; human TRBV7-3 and human TRBV10-3, respectively; human TRBV7-3 and human TRBV7-2, respectively; human TRBV7-3 and human TRBV19, respectively; human TRBV7-3 and human TRBV20-1, respectively; human TRBV7-3 and human TRBV28, respectively; human TRBV19 and human TRBV5-1, respectively; human TRBV19 and human TRBV6-5, respectively; human TRBV19 and human TRBV10-1, respectively; human TRBV19 and human TRBV10-2, respectively; human TRBV19 and human TRBV10-3, respectively; human TRBV19 and human TRBV7-2, respectively; human TRBV19 and human TRBV7-3, respectively; human TRBV19 and human TRBV20-1, respectively; human TRBV19 and human TRBV28, respectively; human TRBV20-1 and human TRBV5-1, respectively; human TRBV20-1 and human TRBV6-5, respectively; human TRBV20-1 and human TRBV10-1, respectively; human TRBV20-1 and human TRBV10-2, respectively; human TRBV20-1 and human TRBV10-3, respectively; human TRBV20-1 and human TRBV7-2, respectively; human TRBV20-1 and human TRBV7-3, respectively; human TRBV20-1 and human TRBV19, respectively; human TRBV20-1 and human TRBV28, respectively; human TRBV28 and human TRBV5-1, respectively; human TRBV28 and human TRBV6-5, respectively; human TRBV28 and human TRBV10-1, respectively; human TRBV28 and human TRBV10-2, respectively; human TRBV28 and human TRBV10-3, respectively; human TRBV28 and human TRBV7-2, respectively; human TRBV28 and human TRBV7-3, respectively; human TRBV28 and human TRBV19, respectively; or human TRBV28 and human TRBV20-1, respectively.

In some embodiments, the first target molecule is human TRAV12, human TRAV13, human TRAV19, human TRAV21, or human TRAV30, and the second target molecule is human TRAV12, human TRAV13, human TRAV19, human TRAV21, or human TRAV30, wherein the first target molecule and the second target molecule are different.

In some embodiments, the first target molecule and the second target molecule are:
human TRAV12 and human TRAV12, respectively;
human TRAV12 and human TRAV13, respectively;
human TRAV12 and human TRAV19, respectively;
human TRAV12 and human TRAV21, respectively;
human TRAV12 and human TRAV30, respectively;
human TRAV13 and human TRAV12, respectively;
human TRAV13 and human TRAV13, respectively;
human TRAV13 and human TRAV19, respectively;
human TRAV13 and human TRAV21, respectively;
human TRAV13 and human TRAV30, respectively;
human TRAV19 and human TRAV12, respectively;
human TRAV19 and human TRAV13, respectively;
human TRAV19 and human TRAV19, respectively;
human TRAV19 and human TRAV21, respectively;
human TRAV19 and human TRAV30, respectively;
human TRAV21 and human TRAV12, respectively;
human TRAV21 and human TRAV13, respectively;
human TRAV21 and human TRAV19, respectively;
human TRAV21 and human TRAV21, respectively;
human TRAV21 and human TRAV30, respectively;
human TRAV30 and human TRAV12, respectively;
human TRAV30 and human TRAV13, respectively;
human TRAV30 and human TRAV19, respectively;
human TRAV30 and human TRAV21, respectively; or
human TRAV30 and human TRAV30, respectively.

In some embodiments, the first target molecule is human TRAV12-2, human TRAV13-1, human TRAV19, human TRAV21, or human TRAV30, and the second target molecule is human TRAV12-2, human TRAV13-1, human TRAV19, human TRAV21, or human TRAV30, wherein the first target molecule and the second target molecule are different.

In some embodiments, the first target molecule and the second target molecule are:
human TRAV12-2 and human TRAV13-1, respectively;
human TRAV12-2 and human TRAV19, respectively;
human TRAV12-2 and human TRAV21, respectively;
human TRAV12-2 and human TRAV30, respectively;
human TRAV13-1 and human TRAV12-2, respectively;
human TRAV13-1 and human TRAV19, respectively;
human TRAV13-1 and human TRAV21, respectively;
human TRAV13-1 and human TRAV30, respectively;
human TRAV19 and human TRAV12-2, respectively;
human TRAV19 and human TRAV13-1, respectively;
human TRAV19 and human TRAV21, respectively;
human TRAV19 and human TRAV30, respectively;
human TRAV21 and human TRAV12-2, respectively;
human TRAV21 and human TRAV13-1, respectively;
human TRAV21 and human TRAV19, respectively;
human TRAV21 and human TRAV30, respectively;
human TRAV30 and human TRAV12-2, respectively;
human TRAV30 and human TRAV13-1, respectively;
human TRAV30 and human TRAV19, respectively; or
human TRAV30 and human TRAV21, respectively.

In some embodiments, the first target molecule is human TRBV5, human TRBV6, human TRBV10, human TRBV7, human TRBV19, human TRBV20, or human TRBV28, and the second target molecule is human TRAV12, human TRAV13, human TRAV19, human TRAV21, or human TRAV30.

In some embodiments, the first target molecule and the second target molecule are:
human TRBV5 and human TRAV12, respectively; human TRBV5 and human TRAV13, respectively; human TRBV5 and human TRAV19, respectively; human TRBV5 and human TRAV21, respectively; human TRBV5 and human TRAV30, respectively; human TRBV6 and human TRAV12, respectively; human TRBV6 and human TRAV13, respectively; human TRBV6 and human TRAV19, respectively; human TRBV6 and human TRAV21, respectively; human TRBV6 and human TRAV30, respectively; human TRBV10 and human TRAV12, respectively; human TRBV10 and human TRAV13, respectively; human TRBV10 and human TRAV19, respectively; human TRBV10 and human TRAV21, respectively; human TRBV10 and human TRAV30, respectively; human TRBV7 and human TRAV12, respectively; human TRBV7 and human TRAV13, respectively; human TRBV7 and human TRAV19, respectively; human TRBV7 and human TRAV21, respectively; human TRBV7 and human TRAV30, respectively; human TRBV19 and human TRAV12, respectively; human TRBV19 and human TRAV13, respectively; human TRBV19 and human TRAV19, respectively; human TRBV19 and human TRAV21, respectively; human TRBV19 and human TRAV30, respectively; human TRBV20 and human TRAV12, respectively; human TRBV20 and human TRAV13, respectively; human TRBV20 and human TRAV19, respectively; human TRBV20 and human TRAV21, respectively; human TRBV20 and human TRAV30, respectively; human TRBV28 and human TRAV12, respectively; human TRBV28 and human TRAV13, respectively; human TRBV28 and human TRAV19, respectively; human TRBV28 and human TRAV21, respectively; or human TRBV28 and human TRAV30, respectively.

In some embodiments, the first target molecule is human TRBV5, human TRBV6, human TRBV10, human TRBV7, human TRBV19, human TRBV20, or human TRBV28, and the second target molecule is human TRAV12-2, human TRAV13-1, human TRAV19, human TRAV21, or human TRAV30.

In some embodiments, the first target molecule and the second target molecule are:
human TRBV5 and human TRAV12-2, respectively; human TRBV5 and human TRAV13-1, respectively; human TRBV5 and human TRAV19, respectively; human TRBV5 and human TRAV21, respectively; human TRBV5 and human TRAV30, respectively; human TRBV6 and human TRAV12-2, respectively; human TRBV6 and human TRAV13-1, respectively; human TRBV6 and human TRAV19, respectively; human TRBV6 and human TRAV21, respectively; human TRBV6 and human TRAV30, respectively; human TRBV10 and human TRAV12-2, respectively; human TRBV10 and human TRAV13-1, respectively; human TRBV10 and human TRAV19, respectively; human TRBV10 and human TRAV21, respectively; human TRBV10 and human TRAV30, respectively; human TRBV7 and human TRAV12-2, respectively; human TRBV7 and human TRAV13-1, respectively; human TRBV7 and human TRAV19, respectively; human TRBV7 and human TRAV21, respectively; human TRBV7 and human TRAV30, respectively; human TRBV19 and human TRAV12-2, respectively; human TRBV19 and human TRAV13-1, respectively; human TRBV19 and human TRAV19, respectively; human TRBV19 and human TRAV21, respectively; human TRBV19 and human TRAV30, respectively; human TRBV20 and human TRAV12-2, respectively; human TRBV20 and human TRAV13-1, respectively; human TRBV20 and human TRAV19, respectively; human TRBV20 and human TRAV21, respectively; human TRBV20 and human TRAV30, respectively; human TRBV28 and human TRAV12-2, respectively; human TRBV28 and human TRAV13-1, respectively; human TRBV28 and human TRAV19, respectively; human TRBV28 and human TRAV21, respectively; or human TRBV28 and human TRAV30, respectively.

In some embodiments, the first target molecule is human TRBV5-1, human TRBV6-5, human TRBV10-1, human TRBV10-2, human TRBV10-3, human TRBV7-2, human TRBV7-3, human TRBV19, human TRBV20-1, or human TRBV28, and the second target molecule is human TRAV12, human TRAV13, human TRAV19, human TRAV21, or human TRAV30.

In some embodiments, the first target molecule and the second target molecule are:
human TRBV5-1 and human TRAV12, respectively; human TRBV5-1 and human TRAV13, respectively; human TRBV5-1 and human TRAV19, respectively; human TRBV5-1 and human TRAV21, respectively; human TRBV5-1 and human TRAV30, respectively; human TRBV6-5 and human TRAV12, respectively; human TRBV6-5 and human TRAV13, respectively; human TRBV6-5 and human TRAV19, respectively; human TRBV6-5 and human TRAV21, respectively; human TRBV6-5 and human TRAV30, respectively; human TRBV10-1 and human TRAV12, respectively; human TRBV10-1 and human TRAV13, respectively; human TRBV10-1 and human TRAV19, respectively; human TRBV10-1 and human TRAV21, respectively; human TRBV10-1 and human TRAV30, respectively; human TRBV10-2 and human TRAV12, respectively; human TRBV10-2 and human TRAV13, respectively; human TRBV10-2 and human TRAV19, respectively; human TRBV10-2 and human TRAV21, respectively; human TRBV10-2 and human TRAV30, respectively; human TRBV10-3 and human TRAV12, respectively; human TRBV10-3 and human TRAV13, respectively; human TRBV10-3 and human TRAV19, respectively; human TRBV10-3 and human TRAV21, respectively; human TRBV10-3 and human TRAV30, respectively; human TRBV7-2 and human TRAV12, respectively; human TRBV7-2 and human TRAV13, respectively; human TRBV7-2 and human TRAV19, respectively; human TRBV7-2 and human TRAV21, respectively; human TRBV7-2 and human TRAV30, respectively; human TRBV7-3 and human TRAV12, respectively; human TRBV7-3 and human TRAV13, respectively; human TRBV7-3 and human TRAV19, respectively; human TRBV7-3 and human TRAV21, respectively; human TRBV7-3 and human TRAV30, respectively; human TRBV19 and human TRAV12, respectively; human TRBV19 and human TRAV13, respectively; human TRBV19 and human TRAV19, respectively; human TRBV19 and human TRAV21, respectively; human TRBV19 and human TRAV30, respectively; human TRBV20-1 and human TRAV12, respectively; human TRBV20-1 and human TRAV13, respectively; human TRBV20-1 and human TRAV19, respectively; human TRBV20-1 and human TRAV21, respectively; human TRBV20-1 and human TRAV30, respectively; human TRBV28 and human TRAV12, respectively; human TRBV28 and human TRAV13, respectively; human TRBV28 and human TRAV19, respectively; human TRBV28 and human TRAV21, respectively; or human TRBV28 and human TRAV30, respectively.

In some embodiments, the first target molecule is human TRBV5-1, human TRBV6-5, human TRBV10-1, human TRBV10-2, human TRBV10-3, human TRBV7-2, human TRBV7-3, human TRBV19, human TRBV20-1, or human TRBV28, and the second target molecule is human TRAV12-2, human TRAV13-1, human TRAV19, human TRAV21, or human TRAV30.

In some embodiments, the first target molecule and the second target molecule are:
human TRBV5-1 and human TRAV12-2, respectively; human TRBV5-1 and human TRAV13-1, respectively; human TRBV5-1 and human TRAV19, respectively; human TRBV5-1 and human TRAV21, respectively; human TRBV5-1 and human TRAV30, respectively; human TRBV6-5 and human TRAV12-2, respectively; human TRBV6-5 and human TRAV13-1, respectively; human TRBV6-5 and human TRAV19, respectively; human TRBV6-5 and human TRAV21, respectively; human TRBV6-5 and human TRAV30, respectively; human TRBV10-1 and human TRAV12-2, respectively; human TRBV10-1 and human TRAV13-1, respectively; human TRBV10-1 and human TRAV19, respectively; human TRBV10-1 and human TRAV21, respectively; human TRBV10-1 and human TRAV30, respectively; human TRBV10-2 and human TRAV12-2, respectively; human TRBV10-2 and human TRAV13-1, respectively; human TRBV10-2 and human TRAV19, respectively; human TRBV10-2 and human TRAV21, respectively; human TRBV10-2 and human TRAV30, respectively; human TRBV10-3 and human TRAV12-2, respectively; human TRBV10-3 and human TRAV13-1, respectively; human TRBV10-3 and human TRAV19, respectively; human TRBV10-3 and human TRAV21, respectively; human TRBV10-3 and human TRAV30, respectively; human TRBV7-2 and human TRAV12-2, respectively; human TRBV7-2 and human TRAV13-1, respectively; human TRBV7-2 and human TRAV19, respectively; human TRBV7-2 and human TRAV21, respectively; human TRBV7-2 and human TRAV30, respectively; human TRBV7-3 and human TRAV12-2, respectively; human TRBV7-3 and human TRAV13-1, respectively; human TRBV7-3 and human TRAV19, respectively; human TRBV7-3 and human TRAV21, respectively; human TRBV7-3 and human TRAV30, respectively; human TRBV19 and human TRAV12-2, respectively; human TRBV19 and human TRAV13-1, respectively; human TRBV19 and human TRAV19, respectively; human TRBV19 and human TRAV21, respectively; human TRBV19 and human TRAV30, respectively; human TRBV20-1 and human TRAV12-2, respectively; human TRBV20-1 and human TRAV13-1, respectively; human TRBV20-1 and human TRAV19, respectively, human TRBV20-1 and human TRAV21, respectively; human TRBV20-1 and human TRAV30, respectively; human TRBV28 and human TRAV12-2, respectively; human TRBV28 and human TRAV13-1, respectively; human TRBV28 and human TRAV19, respectively; human TRBV28 and human TRAV21, respectively; or human TRBV28 and human TRAV30, respectively.

In some embodiments, the first target molecule is human TRAV12, human TRAV13, human TRAV19, human TRAV21, or human TRAV30, and the second target molecule is human TRBV5, human TRBV6, human TRBV10, human TRBV7, human TRBV19, human TRBV20, or human TRBV28.

In some embodiments, the first target molecule and the second target molecule are:
human TRAV12 and human TRBV5, respectively; human TRAV12 and human TRBV6, respectively; human TRAV12 and human TRBV10, respectively; human TRAV12 and human TRBV7, respectively; human TRAV12 and human TRBV19, respectively; human TRAV12 and human TRBV20, respectively; human TRAV12 and human TRBV28, respectively; human TRAV13 and human TRBV5, respectively; human TRAV13 and human TRBV6, respectively; human TRAV13 and human TRBV10, respectively; human TRAV13 and human TRBV7, respectively; human TRAV13 and human TRBV19, respectively; human TRAV13 and human TRBV20, respectively; human TRAV13 and human TRBV28, respectively; human TRAV19 and human TRBV5, respectively; human TRAV19 and human TRBV6, respectively; human TRAV19 and human TRBV10, respectively; human TRAV19 and human TRBV7, respectively; human TRAV19 and human TRBV19, respectively; human TRAV19 and human TRBV20, respectively; human TRAV19 and human TRBV28, respectively; human TRAV21 and human TRBV5, respectively; human TRAV21 and human TRBV6, respectively; human TRAV21 and human TRBV10, respectively; human TRAV21 and human TRBV7, respectively; human TRAV21 and human TRBV19, respectively; human TRAV21 and human TRBV20, respectively; human TRAV21 and human TRBV28, respectively; human TRAV30 and human TRBV5, respectively; human TRAV30 and human TRBV6, respectively; human TRAV30 and human TRBV10, respectively; human TRAV30 and human TRBV7, respectively; human TRAV30 and human TRBV19, respectively; human TRAV30 and human TRBV20, respectively; or human TRAV30 and human TRBV28, respectively.

In some embodiments, the first target molecule is human TRAV12-2, human TRAV13-1, human TRAV19, human TRAV21, or human TRAV30, and the second target molecule is human TRBV5, human TRBV6, human TRBV10, human TRBV7, human TRBV19, human TRBV20, or human TRBV28.

In some embodiments, the first target molecule and the second target molecule are:
human TRAV12-2 and human TRBV5, respectively; human TRAV12-2 and human TRBV6, respectively; human TRAV12-2 and human TRBV10, respectively; human TRAV12-2 and human TRBV7, respectively; human TRAV12-2 and human TRBV19, respectively; human TRAV12-2 and human TRBV20, respectively; human TRAV12-2 and human TRBV28, respectively; human TRAV13-1 and human TRBV5, respectively; human TRAV13-1 and human TRBV6, respectively; human TRAV13-1 and human TRBV10, respectively; human TRAV13-1 and human TRBV7, respectively; human TRAV13-1 and human TRBV19, respectively; human TRAV13-1 and human TRBV20, respectively; human TRAV13-1 and human TRBV28, respectively; human TRAV19 and human TRBV5, respectively; human TRAV19 and human TRBV6, respectively; human TRAV19 and human TRBV10, respectively; human TRAV19 and human TRBV7, respectively; human TRAV19 and human TRBV19, respectively; human TRAV19 and human TRBV20, respectively; human TRAV19 and human TRBV28, respectively; human TRAV21 and human TRBV5, respectively; human TRAV21 and human TRBV6, respectively; human TRAV21 and human TRBV10, respectively; human TRAV21 and human TRBV7, respectively; human TRAV21 and human TRBV19, respectively; human TRAV21 and human TRBV20, respectively; human TRAV21 and human TRBV28, respectively; human TRAV30 and human TRBV5, respectively; human TRAV30 and human TRBV6, respectively; human TRAV30 and human TRBV10, respectively; human TRAV30 and human TRBV7, respectively; human TRAV30 and human TRBV19, respectively; human TRAV30 and human TRBV20, respectively; or human TRAV30 and human TRBV28, respectively.

In some embodiments, the first target molecule is human TRAV12, human TRAV13, human TRAV19, human TRAV21, or human TRAV30, and the second target molecule is human TRBV5-1, human TRBV6-5, human TRBV10-1, human TRBV10-2, human TRBV10-3, human TRBV7-2, human TRBV7-3, human TRBV19, human TRBV20-1, or human TRBV28.

In some embodiments, the first target molecule and the second target molecule are:
human TRAV12 and human TRBV5-1, respectively; human TRAV12 and human TRBV6-5, respectively; human TRAV12 and human TRBV10-1, respectively; human TRAV12 and human TRBV10-2, respectively; human TRAV12 and human TRBV10-3, respectively; human TRAV12 and human TRBV7-2, respectively; human TRAV12 and human TRBV7-3, respectively; human TRAV12 and human TRBV19, respectively; human TRAV12 and human TRBV20-1, respectively; human TRAV12 and human TRBV28, respectively; human TRAV13 and human TRBV5-1, respectively; human TRAV13 and human TRBV6-5, respectively; human TRAV13 and human TRBV10-1, respectively; human TRAV13 and human TRBV10-2, respectively; human TRAV13 and human TRBV10-3, respectively; human TRAV13 and human TRBV7-2, respectively; human TRAV13 and human TRBV7-3, respectively; human TRAV13 and human TRBV19, respectively; human TRAV13 and human TRBV20-1, respectively; human TRAV13 and human TRBV28, respectively; human TRAV19 and human TRBV5-1, respectively; human TRAV19 and human TRBV6-5, respectively; human TRAV19 and human TRBV10-1, respectively; human TRAV19 and human TRBV10-2, respectively; human TRAV19 and human TRBV10-3, respectively; human TRAV19 and human TRBV7-2, respectively; human TRAV19 and human TRBV7-3, respectively; human TRAV19 and human TRBV19, respectively; human TRAV19 and human TRBV20-1, respectively; human TRAV19 and human TRBV28, respectively; human TRAV21 and human TRBV5-1, respectively; human TRAV21 and human TRBV6-5, respectively; human TRAV21 and human TRBV10-1, respectively; human TRAV21 and human TRBV10-2, respectively; human TRAV21 and human TRBV10-3, respectively; human TRAV21 and human TRBV7-2, respectively; human TRAV21 and human TRBV7-3, respectively; human TRAV21 and human TRBV19, respectively; human TRAV21 and human TRBV20-1, respectively; human TRAV21 and human TRBV28, respectively; human TRAV30 and human TRBV5-1, respectively; human TRAV30 and human TRBV6-5, respectively; human TRAV30 and human TRBV10-1, respectively; human TRAV30 and human TRBV10-2, respectively; human TRAV30 and human TRBV10-3, respectively; human TRAV30 and human TRBV7-2, respectively; human TRAV30 and human TRBV7-3, respectively; human TRAV30 and human TRBV19, respectively; human TRAV30 and human TRBV20-1, respectively; or human TRAV30 and human TRBV28, respectively.

In some embodiments, the first target molecule is human TRAV12-2, human TRAV13-1, human TRAV19, human TRAV21, or human TRAV30, and the second target molecule is human TRBV5-1, human TRBV6-5, human TRBV10-1, human TRBV10-2, human TRBV10-3, human TRBV7-2, human TRBV7-3, human TRBV19, human TRBV20-1, or human TRBV28.

In some embodiments, the first target molecule and the second target molecule are:
human TRAV12-2 and human TRBV5-1, respectively; human TRAV12-2 and human TRBV6-5, respectively; human TRAV12-2 and human TRBV10-1, respectively; human TRAV12-2 and human TRBV10-2, respectively; human TRAV12-2 and human TRBV10-3, respectively; human TRAV12-2 and human TRBV7-2, respectively; human TRAV12-2 and human TRBV7-3, respectively; human TRAV12-2 and human TRBV19, respectively; human TRAV12-2 and human TRBV20-1, respectively; human TRAV12-2 and human TRBV28, respectively; human TRAV13-1 and human TRBV5-1, respectively; human TRAV13-1 and human TRBV6-5, respectively; human TRAV13-1 and human TRBV10-1, respectively; human TRAV13-1 and human TRBV10-2, respectively; human TRAV13-1 and human TRBV10-3, respectively; human TRAV13-1 and human TRBV7-2, respectively; human TRAV13-1 and human TRBV7-3, respectively; human TRAV13-1 and human TRBV19, respectively; human TRAV13-1 and human TRBV20-1, respectively; human TRAV13-1 and human TRBV28, respectively; human TRAV19 and human TRBV5-1, respectively; human TRAV19 and human TRBV6-5, respectively; human TRAV19 and human TRBV10-1, respectively; human TRAV19 and human TRBV10-2, respectively; human TRAV19 and human TRBV10-3, respectively; human TRAV19 and human TRBV7-2, respectively; human TRAV19 and human TRBV7-3, respectively; human TRAV19 and human TRBV19, respectively; human TRAV19 and human TRBV20-1, respectively; human TRAV19 and human TRBV28, respectively; human TRAV21 and human TRBV5-1, respectively; human TRAV21 and human TRBV6-5, respectively; human TRAV21 and human TRBV10-1, respectively; human TRAV21 and human TRBV10-2, respectively; human TRAV21 and human TRBV10-3, respectively; human TRAV21 and human TRBV7-2, respectively; human TRAV21 and human TRBV7-3, respectively; human TRAV21 and human TRBV19, respectively; human TRAV21 and human TRBV20-1, respectively; human TRAV21 and human TRBV28, respectively; human TRAV30 and human TRBV5-1, respectively; human TRAV30 and human TRBV6-5, respectively; human TRAV30 and human TRBV10-1, respectively; human TRAV30 and human TRBV10-2, respectively; human TRAV30 and human TRBV10-3, respectively; human TRAV30 and human TRBV7-2, respectively; human TRAV30 and human TRBV7-3, respectively; human TRAV30 and human TRBV19, respectively; human TRAV30 and human TRBV20-1, respectively; or human TRAV30 and human TRBV28, respectively.

In some embodiments, the first binder comprises a full antibody or antigen-binding fragment thereof, a bivalent antibody, bispecific antibody, or a biparatopic antibody.

In some embodiments, the second binder comprises a full antibody or antigen-binding fragment thereof, a bivalent antibody, bispecific antibody, or a biparatopic antibody.

In some embodiments, the antigen-binding fragment is a Fab, F(ab′)2, Fv, a single chain Fv (scFv), a single domain antibody, a diabody (dAb), a single do-main antibody, or a camelid antibody.

In some embodiments, the first binder comprises a heavy chain variable region (VH) comprising a heavy chain complementarity determining region 3 (HC CDR3) comprising the sequence of SEQ ID NO: 3343, SEQ ID NO: 3323, SEQ ID NO: 3353, SEQ ID NO: 1187, SEQ ID NO: 3373, SEQ ID NO: 3383, SEQ ID NO: 3393, SEQ ID NO: 3403, or SEQ ID NO: 3413.

In some embodiments, the first binder comprises a VH comprising a heavy chain complementarity determining region 1 (HC CDR1) comprising the sequence of SEQ ID NO: 3341, SEQ ID NO: 3321, SEQ ID NO: 3351, SEQ ID NO:1190, SEQ ID NO: 3371, SEQ ID NO: 3381, SEQ ID NO: 3391, SEQ ID NO: 3401, SEQ ID NO: 3411, SEQ ID NO: 3421, or SEQ ID NO: 3702.

In some embodiments, the first binder comprises a VH comprising a heavy chain complementarity determining region 2 (HC CDR2) comprising the sequence of SEQ ID NO: 3342, SEQ ID NO: 3322, SEQ ID NO: 3352, SEQ ID NO: 1186, SEQ ID NO: 3372, SEQ ID NO: 3382, SEQ ID NO: 3392, SEQ ID NO: 3402, or SEQ ID NO: 3412.

In some embodiments, the first binder comprises a VH comprising:
(i) a HC CDR1 comprising the sequence of SEQ ID NO: 3341, a HC CDR2 comprising the sequence of SEQ ID NO: 3342, and a HC CDR3 comprising the sequence of SEQ ID NO: 3343;
(ii) a HC CDR1 comprising the sequence of SEQ ID NO: 3321, a HC CDR2 comprising the sequence of SEQ ID NO: 3322, and a HC CDR3 comprising the sequence of SEQ ID NO: 3323;
(iii) a HC CDR1 comprising the sequence of SEQ ID NO: 3351, a HC CDR2 comprising the sequence of SEQ ID NO: 3352, and a HC CDR3 comprising the sequence of SEQ ID NO: 3353;
(iv) a HC CDR1 comprising the sequence of SEQ ID NO: 1190, a HC CDR2 comprising the sequence of SEQ ID NO: 1186, and a HC CDR3 comprising the sequence of SEQ ID NO: 1187;
(v) a HC CDR1 comprising the sequence of SEQ ID NO: 3371, a HC CDR2 comprising the sequence of SEQ ID NO: 3372, and a HC CDR3 comprising the sequence of SEQ ID NO: 3373;
(vi) a HC CDR1 comprising the sequence of SEQ ID NO: 3381, a HC CDR2 comprising the sequence of SEQ ID NO: 3382, and a HC CDR3 comprising the sequence of SEQ ID NO: 3383;
(vii) a HC CDR1 comprising the sequence of SEQ ID NO: 3391, a HC CDR2 comprising the sequence of SEQ ID NO: 3392, and a HC CDR3 comprising the sequence of SEQ ID NO: 3393;
(viii) a HC CDR1 comprising the sequence of SEQ ID NO: 3401, a HC CDR2 comprising the sequence of SEQ ID NO: 3402, and a HC CDR3 comprising the sequence of SEQ ID NO: 3403;
(ix) a HC CDR1 comprising the sequence of SEQ ID NO: 3351, SEQ ID NO: 3421, or SEQ ID NO: 3702, a HC CDR2 comprising the sequence of SEQ ID NO: 3352, and a HC CDR3 comprising the sequence of SEQ ID NO: 3353; or
(x) a HC CDR1 comprising the sequence of SEQ ID NO: 3411, a HC CDR2 comprising the sequence of SEQ ID NO: 3412, and a HC CDR3 comprising the sequence of SEQ ID NO: 3413.

In some embodiments, the first binder comprises a light chain variable region (VL) comprising a light chain complementarity determining region 3 (LC CDR3) comprising the sequence of SEQ ID NO: 3346, SEQ ID NO: 3326, SEQ ID NO: 3356, SEQ ID NO: 1193, SEQ ID NO: 3376, SEQ ID NO: 3386, SEQ ID NO: 3396, SEQ ID NO: 3406, or SEQ ID NO: 3416.

In some embodiments, the first binder comprises a VL comprising a light chain complementarity determining region 1 (LC CDR1) comprising the sequence of SEQ ID NO: 3344, SEQ ID NO: 3324, SEQ ID NO: 3354, SEQ ID NO: 1191, SEQ ID NO: 3374, SEQ ID NO: 3384, SEQ ID NO: 3394, SEQ ID NO: 3404, or SEQ ID NO: 3414.

In some embodiments, the first binder comprises a VL comprising a light chain complementarity determining region 2 (LC CDR2) comprising the sequence of SEQ ID NO: 3345, SEQ ID NO: 3325, SEQ ID NO: 3355, SEQ ID NO:1192, SEQ ID NO: 3375, SEQ ID NO: 3395, SEQ ID NO: 3405, SEQ ID NO: 3415, or SEQ ID NO: 3443.

In some embodiments, the first binder comprises a VL comprising:
(i) a LC CDR1 comprising the sequence of SEQ ID NO: 3344, a LC CDR2 comprising the sequence of SEQ ID NO: 3345, and a LC CDR3 comprising the sequence of SEQ ID NO: 3346;
(ii) a LC CDR1 comprising the sequence of SEQ ID NO: 3324, a LC CDR2 comprising the sequence of SEQ ID NO: 3325, and a LC CDR3 comprising the sequence of SEQ ID NO: 3326;
(iii) a LC CDR1 comprising the sequence of SEQ ID NO: 3354, a LC CDR2 comprising the sequence of SEQ ID NO: 3355, and a LC CDR3 comprising the sequence of SEQ ID NO: 3356;
(iv) a LC CDR1 comprising the sequence of SEQ ID NO: 1191, a LC CDR2 comprising the sequence of SEQ ID NO: 1192, and a LC CDR3 comprising the sequence of SEQ ID NO: 1193;
(v) a LC CDR1 comprising the sequence of SEQ ID NO: 3374, a LC CDR2 comprising the sequence of SEQ ID NO: 3375, and a LC CDR3 comprising the sequence of SEQ ID NO: 3376;
(vi) a LC CDR1 comprising the sequence of SEQ ID NO: 3384, a LC CDR2 comprising the sequence of SEQ ID NO: 3325, and a LC CDR3 comprising the sequence of SEQ ID NO: 3386;
(vii) a LC CDR1 comprising the sequence of SEQ ID NO: 3394, a LC CDR2 comprising the sequence of SEQ ID NO: 3395, and a LC CDR3 comprising the sequence of SEQ ID NO: 3396;
(viii) a LC CDR1 comprising the sequence of SEQ ID NO: 3404, a LC CDR2 comprising the sequence of SEQ ID NO: 3405, and a LC CDR3 comprising the sequence of SEQ ID NO: 3406;
(ix) a LC CDR1 comprising the sequence of SEQ ID NO: 3414, a LC CDR2 comprising the sequence of SEQ ID NO: 3415, and a LC CDR3 comprising the sequence of SEQ ID NO: 3416; or
(x) a LC CDR1 comprising the sequence of SEQ ID NO: 3354, a LC CDR2 comprising the sequence of SEQ ID NO: 3443, and a LC CDR3 comprising the sequence of SEQ ID NO: 3356.

In some embodiments, the first binder comprises:
(i) a VH comprising a HC CDR1 comprising the sequence of SEQ ID NO: 3341, a HC CDR2 comprising the sequence of SEQ ID NO: 3342, a HC CDR3 comprising the sequence of SEQ ID NO: 3343, and a VL comprising a LC CDR1 comprising the sequence of SEQ ID NO: 3344, a LC CDR2 comprising the sequence of SEQ ID NO: 3345, and a LC CDR3 comprising the sequence of SEQ ID NO: 3346;
(ii) a VH comprising a HC CDR1 comprising the sequence of SEQ ID NO: 3321, a HC CDR2 comprising the sequence of SEQ ID NO: 3322, a HC CDR3 comprising the sequence of SEQ ID NO: 3323, and a VL comprising a LC CDR1 comprising the sequence of SEQ ID NO: 3324, a LC CDR2 comprising the sequence of SEQ ID NO: 3325, and a LC CDR3 comprising the sequence of SEQ ID NO: 3326;
(iii) a VH comprising a HC CDR1 comprising the sequence of SEQ ID NO: 3351, a HC CDR2 comprising the sequence of SEQ ID NO: 3352, a HC CDR3 comprising the sequence of SEQ ID NO: 3353, and a VL comprising a LC CDR1 comprising the sequence of SEQ ID NO: 3354, a LC CDR2 comprising the sequence of SEQ ID NO: 3355, and a LC CDR3 comprising the sequence of SEQ ID NO: 3356;
(iv) a VH comprising a HC CDR1 comprising the sequence of SEQ ID NO: 1190, a HC CDR2 comprising the sequence of SEQ ID NO: 1186, a HC CDR3 comprising the sequence of SEQ ID NO: 1187, and a VL comprising a LC CDR1 comprising the sequence of SEQ ID NO: 1191, a LC CDR2 comprising the sequence of SEQ ID NO: 1192, and a LC CDR3 comprising the sequence of SEQ ID NO: 1193;
(v) a VH comprising a HC CDR1 comprising the sequence of SEQ ID NO: 3371, a HC CDR2 comprising the sequence of SEQ ID NO: 3372, a HC CDR3 comprising the sequence of SEQ ID NO: 3373, and a VL comprising a LC CDR1 comprising the sequence of SEQ ID NO: 3374, a LC CDR2 comprising the sequence of SEQ ID NO: 3375, and a LC CDR3 comprising the sequence of SEQ ID NO: 3376;
(vi) a VH comprising a HC CDR1 comprising the sequence of SEQ ID NO: 3381, a HC CDR2 comprising the sequence of SEQ ID NO: 3382, a HC CDR3 comprising the sequence of SEQ ID NO: 3383, and a VL comprising a LC CDR1 comprising the sequence of SEQ ID NO: 3384, a LC CDR2 comprising the sequence of SEQ ID NO: 3325, and a LC CDR3 comprising the sequence of SEQ ID NO: 3386;
(vi) a VH comprising a HC CDR1 comprising the sequence of SEQ ID NO: 3391, a HC CDR2 comprising the sequence of SEQ ID NO: 3392, a HC CDR3 comprising the sequence of SEQ ID NO: 3393, and a VL comprising a LC CDR1 comprising the sequence of SEQ ID NO: 3394, a LC CDR2 comprising the sequence of SEQ ID NO: 3395, and a LC CDR3 comprising the sequence of SEQ ID NO: 3396;
(vii) a VH comprising a HC CDR1 comprising the sequence of SEQ ID NO: 3401, a HC CDR2 comprising the sequence of SEQ ID NO: 3402, a HC CDR3 comprising the sequence of SEQ ID NO: 3403, and a VL comprising a LC CDR1 comprising the sequence of SEQ ID NO: 3404, a LC CDR2 comprising the sequence of SEQ ID NO: 3405, and a LC CDR3 comprising the sequence of SEQ ID NO: 3406;
(viii) a VH comprising a HC CDR1 comprising the sequence of SEQ ID NO: 3411, a HC CDR2 comprising the sequence of SEQ ID NO: 3412, a HC CDR3 comprising the sequence of SEQ ID NO: 3413, and a VL comprising a LC CDR1 comprising the sequence of SEQ ID NO: 3414, a LC CDR2 comprising the sequence of SEQ ID NO: 3415, and a LC CDR3 comprising the sequence of SEQ ID NO: 3416;
(ix) a VH comprising a HC CDR1 comprising the sequence of SEQ ID NO: 3351, SEQ ID NO: 3421, or SEQ ID NO: 3702, a HC CDR2 comprising the sequence of SEQ ID NO: 3352, a HC CDR3 comprising the sequence of SEQ ID NO: 3353, and a VL comprising a LC CDR1 comprising the sequence of SEQ ID NO: 3354, a LC CDR2 comprising the sequence of SEQ ID NO: 3355, and a LC CDR3 comprising the sequence of SEQ ID NO: 3356; or
(x) a VH comprising a HC CDR1 comprising the sequence of SEQ ID NO: 3351, SEQ ID NO: 3421, or SEQ ID NO: 3702, a HC CDR2 comprising the sequence of SEQ ID NO: 3352, a HC CDR3 comprising the sequence of SEQ ID NO: 3353, and a VL comprising a LC CDR1 comprising the sequence of SEQ ID NO: 3354, a LC CDR2 comprising the sequence of SEQ ID NO: 3443, and a LC CDR3 comprising the sequence of SEQ ID NO: 3356.

In some embodiments, the first binder comprises a VH comprising a sequence having at least 75% sequence identity to any one sequence selected from the group consisting of: SEQ ID NO: 3445, SEQ ID NO: 3448, SEQ ID NO: 3450, SEQ ID NO: 1346, SEQ ID NO: 1196, SEQ ID NO: 3455, SEQ ID NO: 3457, SEQ ID NO: 3463, SEQ ID NO: 3465, and SEQ ID NO: 3470.

In some embodiments, the first binder comprises a VH comprising any one sequence selected from the group consisting of: SEQ ID NO: 3445, SEQ ID NO: 3448, SEQ ID NO: 3450, SEQ ID NO: 1346, SEQ ID NO: 1196, SEQ ID NO: 3455, SEQ ID NO: 3457, SEQ ID NO: 3463, SEQ ID NO: 3465, and SEQ ID NO: 3470.

In some embodiments, the first binder comprises a VL comprising a sequence having at least 75% sequence identity to any one sequence selected from the group consisting of: SEQ ID NO: 3446, SEQ ID NO: 3449, SEQ ID NO: 3451, SEQ ID NO: 1349, SEQ ID NO:1195, SEQ ID NO: 3456, SEQ ID NO: 3458, SEQ ID NO: 3464, SEQ ID NO: 3466, SEQ ID NO: 3471, SEQ ID NO: 3475, and SEQ ID NO: 3476.

In some embodiments, the first binder comprises a VL comprising any one sequence selected from the group consisting of: SEQ ID NO: 3446, SEQ ID NO: 3449, SEQ ID NO: 3451, SEQ ID NO: 1349, SEQ ID NO:1195, SEQ ID NO: 3456, SEQ ID NO: 3458, SEQ ID NO: 3464, SEQ ID NO: 3466, SEQ ID NO: 3471, SEQ ID NO: 3475, and SEQ ID NO: 3476.

In some embodiments, the first binder comprises:
(i) a VH comprising a sequence having at least 75% sequence identity to any one sequence selected from the group consisting of: SEQ ID NO: 3445, SEQ ID NO: 3448, SEQ ID NO: 3450, SEQ ID NO: 1346, SEQ ID NO: 1196, SEQ ID NO: 3455, SEQ ID NO: 3457, SEQ ID NO: 3463, SEQ ID NO: 3465, and SEQ ID NO: 3470; and
(ii) a VL comprising a sequence having at least 75% sequence identity to any one sequence selected from the group consisting of: SEQ ID NO: 3446, SEQ ID NO: 3449, SEQ ID NO: 3451, SEQ ID NO: 1349, SEQ ID NO:1195, SEQ ID NO: 3456, SEQ ID NO: 3458, SEQ ID NO: 3464, SEQ ID NO: 3466, SEQ ID NO: 3471, SEQ ID NO: 3475, and SEQ ID NO: 3476.

In some embodiments, the first binder comprises:
(i) a VH comprising any one sequence selected from the group consisting of: SEQ ID NO: 3445, SEQ ID NO: 3448, SEQ ID NO: 3450, SEQ ID NO: 1346, SEQ ID NO: 1196, SEQ ID NO: 3455, SEQ ID NO: 3457, SEQ ID NO: 3463, SEQ ID NO: 3465, and SEQ ID NO: 3470; and
(ii) a VL comprising any one sequence selected from the group consisting of: SEQ ID NO: 3446, SEQ ID NO: 3449, SEQ ID NO: 3451, SEQ ID NO: 1349, SEQ ID NO:1195, SEQ ID NO: 3456, SEQ ID NO: 3458, SEQ ID NO: 3464, SEQ ID NO: 3466, SEQ ID NO: 3471, SEQ ID NO: 3475, and SEQ ID NO: 3476.

In some embodiments, the first binder comprises:
(i) a VH comprising a sequence having at least 75% sequence identity to the sequence of SEQ ID NO: 3445, and a VL comprising a sequence having at least 75% sequence identity to the sequence of SEQ ID NO: 3446;
(ii) a VH comprising a sequence having at least 75% sequence identity to the sequence of SEQ ID NO: 3448, and a VL comprising a sequence having at least 75% sequence identity to the sequence of SEQ ID NO: 3449;
(iii) a VH comprising a sequence having at least 75% sequence identity to the sequence of SEQ ID NO: 3450, and a VL comprising a sequence having at least 75% sequence identity to the sequence of SEQ ID NO: 3451;
(iv) a VH comprising a sequence having at least 75% sequence identity to the sequence of SEQ ID NO: 1346, and a VL comprising a sequence having at least 75% sequence identity to the sequence of SEQ ID NO: 1349;
(v) a VH comprising a sequence having at least 75% sequence identity to the sequence of SEQ ID NO: 1196, and a VL comprising a sequence having at least 75% sequence identity to the sequence of SEQ ID NO: 1195;
(v) a VH comprising a sequence having at least 75% sequence identity to the sequence of SEQ ID NO: 3455, and a VL comprising a sequence having at least 75% sequence identity to the sequence of SEQ ID NO: 3456;
(vi) a VH comprising a sequence having at least 75% sequence identity to the sequence of SEQ ID NO: 3457, and a VL comprising a sequence having at least 75% sequence identity to the sequence of SEQ ID NO: 3458;
(vii) a VH comprising a sequence having at least 75% sequence identity to the sequence of SEQ ID NO: 3463, and a VL comprising a sequence having at least 75% sequence identity to the sequence of SEQ ID NO: 3464;
(viii) a VH comprising a sequence having at least 75% sequence identity to the sequence of SEQ ID NO: 3465, and a VL comprising a sequence having at least 75% sequence identity to the sequence of SEQ ID NO: 3466;
(ix) a VH comprising a sequence having at least 75% sequence identity to the sequence of SEQ ID NO: 3470, and a VL comprising a sequence having at least 75% sequence identity to the sequence of SEQ ID NO: 3471;
(x) a VH comprising a sequence having at least 75% sequence identity to the sequence of SEQ ID NO: 1346, and a VL comprising a sequence having at least 75% sequence identity to the sequence of SEQ ID NO: 3475; or
(xi) a VH comprising a sequence having at least 75% sequence identity to the sequence of SEQ ID NO: 1346, and a VL comprising a sequence having at least 75% sequence identity to the sequence of SEQ ID NO: 3476.

In some embodiments, the first binder comprises:
(i) a VH comprising the sequence of SEQ ID NO: 3445, and a VL comprising the sequence of SEQ ID NO: 3446;
(ii) a VH comprising the sequence of SEQ ID NO: 3448, and a VL comprising the sequence of SEQ ID NO: 3449;
(iii) a VH comprising the sequence of SEQ ID NO: 3450, and a VL comprising the sequence of SEQ ID NO: 3451;
(iv) a VH comprising the sequence of SEQ ID NO: 1346, and a VL comprising the sequence of SEQ ID NO: 1349;
(v) a VH comprising the sequence of SEQ ID NO: 1196, and a VL comprising the sequence of SEQ ID NO: 1195;
(vi) a VH comprising the sequence of SEQ ID NO: 3455, and a VL comprising the sequence of SEQ ID NO: 3456;
(vii) a VH comprising the sequence of SEQ ID NO: 3457, and a VL comprising the sequence of SEQ ID NO: 3458;
(viii) a VH comprising the sequence of SEQ ID NO: 3463, and a VL comprising the sequence of SEQ ID NO: 3464;
(ix) a VH comprising the sequence of SEQ ID NO: 3465, and a VL comprising the sequence of SEQ ID NO: 3466;
(x) a VH comprising the sequence of SEQ ID NO: 3470, and a VL comprising the sequence of SEQ ID NO: 3471;
(xi) a VH comprising the sequence of SEQ ID NO: 1346, and a VL comprising the sequence of SEQ ID NO: 3475; or
(xii) a VH comprising the sequence of SEQ ID NO: 1346, and a VL comprising the sequence of SEQ ID NO: 3476.

In some embodiments, the second binder comprises a heavy chain variable region (VH) comprising a heavy chain complementarity determining region 3 (HC CDR3) comprising the sequence of SEQ ID NO: 3343, SEQ ID NO: 3323, SEQ ID NO: 3353, SEQ ID NO: 1187, SEQ ID NO: 3373, SEQ ID NO: 3383, SEQ ID NO: 3393, SEQ ID NO: 3403, or SEQ ID NO: 3413.

In some embodiments, the second binder comprises a VH comprising a heavy chain complementarity determining region 1 (HC CDR1) comprising the sequence of SEQ ID NO: 3341, SEQ ID NO: 3321, SEQ ID NO: 3351, SEQ ID NO:1190, SEQ ID NO: 3371, SEQ ID NO: 3381, SEQ ID NO: 3391, SEQ ID NO: 3401, SEQ ID NO: 3411, SEQ ID NO: 3421, or SEQ ID NO: 3702.

In some embodiments, the second binder comprises a VH comprising a heavy chain complementarity determining region 2 (HC CDR2) comprising the sequence of SEQ ID NO: 3342, SEQ ID NO: 3322, SEQ ID NO: 3352, SEQ ID NO: 1186, SEQ ID NO: 3372, SEQ ID NO: 3382, SEQ ID NO: 3392, SEQ ID NO: 3402, or SEQ ID NO: 3412.

In some embodiments, the second binder comprises a VH comprising:
(i) a HC CDR1 comprising the sequence of SEQ ID NO: 3341, a HC CDR2 comprising the sequence of SEQ ID NO: 3342, and a HC CDR3 comprising the sequence of SEQ ID NO: 3343;
(ii) a HC CDR1 comprising the sequence of SEQ ID NO: 3321, a HC CDR2 comprising the sequence of SEQ ID NO: 3322, and a HC CDR3 comprising the sequence of SEQ ID NO: 3323;
(iii) a HC CDR1 comprising the sequence of SEQ ID NO: 3351, a HC CDR2 comprising the sequence of SEQ ID NO: 3352, and a HC CDR3 comprising the sequence of SEQ ID NO: 3353;
(iv) a HC CDR1 comprising the sequence of SEQ ID NO: 1190, a HC CDR2 comprising the sequence of SEQ ID NO: 1186, and a HC CDR3 comprising the sequence of SEQ ID NO: 1187;
(v) a HC CDR1 comprising the sequence of SEQ ID NO: 3371, a HC CDR2 comprising the sequence of SEQ ID NO: 3372, and a HC CDR3 comprising the sequence of SEQ ID NO: 3373;
(vi) a HC CDR1 comprising the sequence of SEQ ID NO: 3381, a HC CDR2 comprising the sequence of SEQ ID NO: 3382, and a HC CDR3 comprising the sequence of SEQ ID NO: 3383;
(vii) a HC CDR1 comprising the sequence of SEQ ID NO: 3391, a HC CDR2 comprising the sequence of SEQ ID NO: 3392, and a HC CDR3 comprising the sequence of SEQ ID NO: 3393;
(viii) a HC CDR1 comprising the sequence of SEQ ID NO: 3401, a HC CDR2 comprising the sequence of SEQ ID NO: 3402, and a HC CDR3 comprising the sequence of SEQ ID NO: 3403;
(ix) a HC CDR1 comprising the sequence of SEQ ID NO: 3351, SEQ ID NO: 3421, or SEQ ID NO: 3702, a HC CDR2 comprising the sequence of SEQ ID NO: 3352, and a HC CDR3 comprising the sequence of SEQ ID NO: 3353; or
(x) a HC CDR1 comprising the sequence of SEQ ID NO: 3411, a HC CDR2 comprising the sequence of SEQ ID NO: 3412, and a HC CDR3 comprising the sequence of SEQ ID NO: 3413.

In some embodiments, the second binder comprises a light chain variable region (VL) comprising a light chain complementarity determining region 3 (LC CDR3) comprising the sequence of SEQ ID NO: 3346, SEQ ID NO: 3326, SEQ ID NO: 3356, SEQ ID NO: 1193, SEQ ID NO: 3376, SEQ ID NO: 3386, SEQ ID NO: 3396, SEQ ID NO: 3406, or SEQ ID NO: 3416.

In some embodiments, the second binder comprises a VL comprising a light chain complementarity determining region 1 (LC CDR1) comprising the sequence of SEQ ID NO: 3344, SEQ ID NO: 3324, SEQ ID NO: 3354, SEQ ID NO: 1191, SEQ ID NO: 3374, SEQ ID NO: 3384, SEQ ID NO: 3394, SEQ ID NO: 3404, or SEQ ID NO: 3414.

In some embodiments, the second binder comprises a VL comprising a light chain complementarity determining region 2 (LC CDR2) comprising the sequence of SEQ ID NO: 3345, SEQ ID NO: 3325, SEQ ID NO: 3355, SEQ ID NO:1192, SEQ ID NO: 3375, SEQ ID NO: 3395, SEQ ID NO: 3405, SEQ ID NO: 3415, or SEQ ID NO: 3443.

In some embodiments, the second binder comprises a VL comprising:
(i) a LC CDR1 comprising the sequence of SEQ ID NO: 3344, a LC CDR2 comprising the sequence of SEQ ID NO: 3345, and a LC CDR3 comprising the sequence of SEQ ID NO: 3346;
(ii) a LC CDR1 comprising the sequence of SEQ ID NO: 3324, a LC CDR2 comprising the sequence of SEQ ID NO: 3325, and a LC CDR3 comprising the sequence of SEQ ID NO: 3326;
(iii) a LC CDR1 comprising the sequence of SEQ ID NO: 3354, a LC CDR2 comprising the sequence of SEQ ID NO: 3355, and a LC CDR3 comprising the sequence of SEQ ID NO: 3356;
(iv) a LC CDR1 comprising the sequence of SEQ ID NO: 1191, a LC CDR2 comprising the sequence of SEQ ID NO: 1192, and a LC CDR3 comprising the sequence of SEQ ID NO: 1193;
(v) a LC CDR1 comprising the sequence of SEQ ID NO: 3374, a LC CDR2 comprising the sequence of SEQ ID NO: 3375, and a LC CDR3 comprising the sequence of SEQ ID NO: 3376;
(vi) a LC CDR1 comprising the sequence of SEQ ID NO: 3384, a LC CDR2 comprising the sequence of SEQ ID NO: 3325, and a LC CDR3 comprising the sequence of SEQ ID NO: 3386;
(vii) a LC CDR1 comprising the sequence of SEQ ID NO: 3394, a LC CDR2 comprising the sequence of SEQ ID NO: 3395, and a LC CDR3 comprising the sequence of SEQ ID NO: 3396;
(viii) a LC CDR1 comprising the sequence of SEQ ID NO: 3404, a LC CDR2 comprising the sequence of SEQ ID NO: 3405, and a LC CDR3 comprising the sequence of SEQ ID NO: 3406;
(ix) a LC CDR1 comprising the sequence of SEQ ID NO: 3414, a LC CDR2 comprising the sequence of SEQ ID NO: 3415, and a LC CDR3 comprising the sequence of SEQ ID NO: 3416; or
(x) a LC CDR1 comprising the sequence of SEQ ID NO: 3354, a LC CDR2 comprising the sequence of SEQ ID NO: 3443, and a LC CDR3 comprising the sequence of SEQ ID NO: 3356.

In some embodiments, the second binder comprises:
(i) a VH comprising a HC CDR1 comprising the sequence of SEQ ID NO: 3341, a HC CDR2 comprising the sequence of SEQ ID NO: 3342, a HC CDR3 comprising the sequence of SEQ ID NO: 3343, and a VL comprising a LC CDR1 comprising the sequence of SEQ ID NO: 3344, a LC CDR2 comprising the sequence of SEQ ID NO: 3345, and a LC CDR3 comprising the sequence of SEQ ID NO: 3346;
(ii) a VH comprising a HC CDR1 comprising the sequence of SEQ ID NO: 3321, a HC CDR2 comprising the sequence of SEQ ID NO: 3322, a HC CDR3 comprising the sequence of SEQ ID NO: 3323, and a VL comprising a LC CDR1 comprising the sequence of SEQ ID NO: 3324, a LC CDR2 comprising the sequence of SEQ ID NO: 3325, and a LC CDR3 comprising the sequence of SEQ ID NO: 3326;
(iii) a VH comprising a HC CDR1 comprising the sequence of SEQ ID NO: 3351, a HC CDR2 comprising the sequence of SEQ ID NO: 3352, a HC CDR3 comprising the sequence of SEQ ID NO: 3353, and a VL comprising a LC CDR1 comprising the sequence of SEQ ID NO: 3354, a LC CDR2 comprising the sequence of SEQ ID NO: 3355, and a LC CDR3 comprising the sequence of SEQ ID NO: 3356;
(iv) a VH comprising a HC CDR1 comprising the sequence of SEQ ID NO: 1190, a HC CDR2 comprising the sequence of SEQ ID NO: 1186, a HC CDR3 comprising the sequence of SEQ ID NO: 1187, and a VL comprising a LC CDR1 comprising the sequence of SEQ ID NO: 1191, a LC CDR2 comprising the sequence of SEQ ID NO: 1192, and a LC CDR3 comprising the sequence of SEQ ID NO: 1193;
(v) a VH comprising a HC CDR1 comprising the sequence of SEQ ID NO: 3371, a HC CDR2 comprising the sequence of SEQ ID NO: 3372, a HC CDR3 comprising the sequence of SEQ ID NO: 3373, and a VL comprising a LC CDR1 comprising the sequence of SEQ ID NO: 3374, a LC CDR2 comprising the sequence of SEQ ID NO: 3375, and a LC CDR3 comprising the sequence of SEQ ID NO: 3376;
(vi) a VH comprising a HC CDR1 comprising the sequence of SEQ ID NO: 3381, a HC CDR2 comprising the sequence of SEQ ID NO: 3382, a HC CDR3 comprising the sequence of SEQ ID NO: 3383, and a VL comprising a LC CDR1 comprising the sequence of SEQ ID NO: 3384, a LC CDR2 comprising the sequence of SEQ ID NO: 3325, and a LC CDR3 comprising the sequence of SEQ ID NO: 3386;
(vii) a VH comprising a HC CDR1 comprising the sequence of SEQ ID NO: 3391, a HC CDR2 comprising the sequence of SEQ ID NO: 3392, a HC CDR3 comprising the sequence of SEQ ID NO: 3393, and a VL comprising a LC CDR1 comprising the sequence of SEQ ID NO: 3394, a LC CDR2 comprising the sequence of SEQ ID NO: 3395, and a LC CDR3 comprising the sequence of SEQ ID NO: 3396;
(viii) a VH comprising a HC CDR1 comprising the sequence of SEQ ID NO: 3401, a HC CDR2 comprising the sequence of SEQ ID NO: 3402, a HC CDR3 comprising the sequence of SEQ ID NO: 3403, and a VL comprising a LC CDR1 comprising the sequence of SEQ ID NO: 3404, a LC CDR2 comprising the sequence of SEQ ID NO: 3405, and a LC CDR3 comprising the sequence of SEQ ID NO: 3406;
(ix) a VH comprising a HC CDR1 comprising the sequence of SEQ ID NO: 3411, a HC CDR2 comprising the sequence of SEQ ID NO: 3412, a HC CDR3 comprising the sequence of SEQ ID NO: 3413, and a VL comprising a LC CDR1 comprising the sequence of SEQ ID NO: 3414, a LC CDR2 comprising the sequence of SEQ ID NO: 3415, and a LC CDR3 comprising the sequence of SEQ ID NO: 3416;
(x) a VH comprising a HC CDR1 comprising the sequence of SEQ ID NO: 3351, SEQ ID NO: 3421, or SEQ ID NO: 3702, a HC CDR2 comprising the sequence of SEQ ID NO: 3352, a HC CDR3 comprising the sequence of SEQ ID NO: 3353, and a VL comprising a LC CDR1 comprising the sequence of SEQ ID NO: 3354, a LC CDR2 comprising the sequence of SEQ ID NO: 3355, and a LC CDR3 comprising the sequence of SEQ ID NO: 3356; or
(xi) a VH comprising a HC CDR1 comprising the sequence of SEQ ID NO: 3351, SEQ ID NO: 3421, or SEQ ID NO: 3702, a HC CDR2 comprising the sequence of SEQ ID NO: 3352, a HC CDR3 comprising the sequence of SEQ ID NO: 3353, and a VL comprising a LC CDR1 comprising the sequence of SEQ ID NO: 3354, a LC CDR2 comprising the sequence of SEQ ID NO: 3443, and a LC CDR3 comprising the sequence of SEQ ID NO: 3356.

In some embodiments, the second binder comprises a VH comprising a sequence having at least 75% sequence identity to any one sequence selected from the group consisting of: SEQ ID NO: 3445, SEQ ID NO: 3448, SEQ ID NO: 3450, SEQ ID NO: 1346, SEQ ID NO: 1196, SEQ ID NO: 3455, SEQ ID NO: 3457, SEQ ID NO: 3463, SEQ ID NO: 3465, and SEQ ID NO: 3470.

In some embodiments, the second binder comprises a VH comprising any one sequence selected from the group consisting of: SEQ ID NO: 3445, SEQ ID NO: 3448, SEQ ID NO: 3450, SEQ ID NO: 1346, SEQ ID NO: 1196, SEQ ID NO: 3455, SEQ ID NO: 3457, SEQ ID NO: 3463, SEQ ID NO: 3465, and SEQ ID NO: 3470.

In some embodiments, the second binder comprises a VL comprising a sequence having at least 75% sequence identity to any one sequence selected from the group consisting of: SEQ ID NO: 3446, SEQ ID NO: 3449, SEQ ID NO: 3451, SEQ ID NO: 1349, SEQ ID NO: 1195, SEQ ID NO: 3456, SEQ ID NO: 3458, SEQ ID NO: 3464, SEQ ID NO: 3466, SEQ ID NO: 3471, SEQ ID NO: 3475, and SEQ ID NO: 3476.

In some embodiments, the second binder comprises a VL comprising any one sequence selected from the group consisting of: SEQ ID NO: 3446, SEQ ID NO: 3449, SEQ ID NO: 3451, SEQ ID NO: 1349, SEQ ID NO:1195, SEQ ID NO: 3456, SEQ ID NO: 3458, SEQ ID NO: 3464, SEQ ID NO: 3466, SEQ ID NO: 3471, SEQ ID NO: 3475, and SEQ ID NO: 3476.

In some embodiments, the second binder comprises:
(i) a VH comprising a sequence having at least 75% sequence identity to any one sequence selected from the group consisting of: SEQ ID NO: 3445, SEQ ID NO: 3448, SEQ ID NO: 3450, SEQ ID NO: 1346, SEQ ID NO: 1196, SEQ ID NO: 3455, SEQ ID NO: 3457, SEQ ID NO: 3463, SEQ ID NO: 3465, and SEQ ID NO: 3470; and
(ii) a VL comprising a sequence having at least 75% sequence identity to any one sequence selected from the group consisting of: SEQ ID NO: 3446, SEQ ID NO: 3449, SEQ ID NO: 3451, SEQ ID NO: 1349, SEQ ID NO:1195, SEQ ID NO: 3456, SEQ ID NO: 3458, SEQ ID NO: 3464, SEQ ID NO: 3466, SEQ ID NO: 3471, SEQ ID NO: 3475, and SEQ ID NO: 3476.

In some embodiments, the second binder comprises:
(i) a VH comprising any one sequence selected from the group consisting of: SEQ ID NO: 3445, SEQ ID NO: 3448, SEQ ID NO: 3450, SEQ ID NO: 1346, SEQ ID NO: 1196, SEQ ID NO: 3455, SEQ ID NO: 3457, SEQ ID NO: 3463, SEQ ID NO: 3465, and SEQ ID NO: 3470; and
(ii) a VL comprising any one sequence selected from the group consisting of: SEQ ID NO: 3446, SEQ ID NO: 3449, SEQ ID NO: 3451, SEQ ID NO: 1349, SEQ ID NO:1195, SEQ ID NO: 3456, SEQ ID NO: 3458, SEQ ID NO: 3464, SEQ ID NO: 3466, SEQ ID NO: 3471, SEQ ID NO: 3475, and SEQ ID NO: 3476.

In some embodiments, the second binder comprises:
(i) a VH comprising a sequence having at least 75% sequence identity to the sequence of SEQ ID NO: 3445, and a VL comprising a sequence having at least 75% sequence identity to the sequence of SEQ ID NO: 3446;
(ii) a VH comprising a sequence having at least 75% sequence identity to the sequence of SEQ ID NO: 3448, and a VL comprising a sequence having at least 75% sequence identity to the sequence of SEQ ID NO: 3449;
(iii) a VH comprising a sequence having at least 75% sequence identity to the sequence of SEQ ID NO: 3450, and a VL comprising a sequence having at least 75% sequence identity to the sequence of SEQ ID NO: 3451;
(iv) a VH comprising a sequence having at least 75% sequence identity to the sequence of SEQ ID NO: 1346, and a VL comprising a sequence having at least 75% sequence identity to the sequence of SEQ ID NO: 1349;
(v) a VH comprising a sequence having at least 75% sequence identity to the sequence of SEQ ID NO: 1196, and a VL comprising a sequence having at least 75% sequence identity to the sequence of SEQ ID NO: 1195;
(vi) a VH comprising a sequence having at least 75% sequence identity to the sequence of SEQ ID NO: 3455, and a VL comprising a sequence having at least 75% sequence identity to the sequence of SEQ ID NO: 3456;
(vii) a VH comprising a sequence having at least 75% sequence identity to the sequence of SEQ ID NO: 3457, and a VL comprising a sequence having at least 75% sequence identity to the sequence of SEQ ID NO: 3458;
(viii) a VH comprising a sequence having at least 75% sequence identity to the sequence of SEQ ID NO: 3463, and a VL comprising a sequence having at least 75% sequence identity to the sequence of SEQ ID NO: 3464;
(ix) a VH comprising a sequence having at least 75% sequence identity to the sequence of SEQ ID NO: 3465, and a VL comprising a sequence having at least 75% sequence identity to the sequence of SEQ ID NO: 3466;
(x) a VH comprising a sequence having at least 75% sequence identity to the sequence of SEQ ID NO: 3470, and a VL comprising a sequence having at least 75% sequence identity to the sequence of SEQ ID NO: 3471;
(xi) a VH comprising a sequence having at least 75% sequence identity to the sequence of SEQ ID NO: 1346, and a VL comprising a sequence having at least 75% sequence identity to the sequence of SEQ ID NO: 3475; or
(xii) a VH comprising a sequence having at least 75% sequence identity to the sequence of SEQ ID NO: 1346, and a VL comprising a sequence having at least 75% sequence identity to the sequence of SEQ ID NO: 3476.

In some embodiments, the second binder comprises:
(i) a VH comprising the sequence of SEQ ID NO: 3445, and a VL comprising the sequence of SEQ ID NO: 3446;
(ii) a VH comprising the sequence of SEQ ID NO: 3448, and a VL comprising the sequence of SEQ ID NO: 3449;
(iii) a VH comprising the sequence of SEQ ID NO: 3450, and a VL comprising the sequence of SEQ ID NO: 3451;
(iv) a VH comprising the sequence of SEQ ID NO: 1346, and a VL comprising the sequence of SEQ ID NO: 1349;
(v) a VH comprising the sequence of SEQ ID NO: 1196, and a VL comprising the sequence of SEQ ID NO: 1195;
(vi) a VH comprising the sequence of SEQ ID NO: 3455, and a VL comprising the sequence of SEQ ID NO: 3456;
(vii) a VH comprising the sequence of SEQ ID NO: 3457, and a VL comprising the sequence of SEQ ID NO: 3458;
(viii) a VH comprising the sequence of SEQ ID NO: 3463, and a VL comprising the sequence of SEQ ID NO: 3464;
(ix) a VH comprising the sequence of SEQ ID NO: 3465, and a VL comprising the sequence of SEQ ID NO: 3466;
(x) a VH comprising the sequence of SEQ ID NO: 3470, and a VL comprising the sequence of SEQ ID NO: 3471;
(xi) a VH comprising the sequence of SEQ ID NO: 1346, and a VL comprising the sequence of SEQ ID NO: 3475; or
(xii) a VH comprising the sequence of SEQ ID NO: 1346, and a VL comprising the sequence of SEQ ID NO: 3476.

In some embodiments, the first binder and the second binder bind to different TCRβV regions.

In some embodiments, the first binder and the second binder bind to different TCRβVs belonging to different subfamilies.

In some embodiments, the first binder and the second binder bind to different members of the same TCRβV subfamily.

In some embodiments, the first binder comprises one or more heavy chain constant regions selected from the group consisting of IgG1 heavy chain constant region or fragment thereof, IgG2 heavy chain constant region or fragment thereof, IgG3 heavy chain constant region or fragment thereof, IgGA1 heavy chain constant region or fragment thereof, IgGA2 heavy chain constant region or fragment thereof, IgG4 heavy chain constant region or fragment thereof, IgJ heavy chain constant region or fragment thereof, IgM heavy chain constant region or fragment thereof, IgD heavy chain constant region or fragment thereof, and IgE heavy chain constant region or fragment thereof.

In some embodiments, the first binder comprises a kappa light chain constant region or fragment thereof, a lambda light chain constant region or fragment thereof, or a combination thereof.

In some embodiments, the second binder comprises one or more heavy chain constant regions selected from the group consisting of IgG1 heavy chain constant region or fragment thereof, IgG2 heavy chain constant region or fragment there-of. IgG3 heavy chain constant region or fragment thereof, IgGA1 heavy chain constant region or fragment thereof, IgGA2 heavy chain constant region or fragment thereof. IgG4 heavy chain constant region or fragment thereof, IgJ heavy chain constant region or fragment thereof. IgM heavy chain constant region or fragment thereof, IgD heavy chain constant region or fragment thereof, and IgE heavy chain constant region or fragment thereof.

In some embodiments, the second binder comprises a kappa light chain constant region or fragment thereof, a lambda light chain constant region or fragment thereof, or a combination thereof.

In some embodiments, the first agent is a multifunctional molecule.

In some embodiments, the first agent is a multispecific molecule.

In some embodiments, the second agent is a multifunctional molecule.

In some embodiments, the second agent is a multispecific molecule.

In some embodiments, the first agent, the second agent, or a combination thereof comprises at least two non-contiguous polypeptide chains; wherein a first polypeptide chain of the at least two non-contiguous polypeptide chains comprises a first Fc region, and a second polypeptide chain of the at least two non-contiguous polypeptide chains comprises a second Fc region; and wherein the first Fc region and the second Fc region comprise an Fc interface with a knob-in-a hole.

In some embodiments, the first Fc region, the second Fc region, or a combination thereof comprises an Asn297Ala mutation, a Leu234Ala/Leu235Ala mutation, or a combination thereof.

In some embodiments, the first agent is a multispecific molecule that further comprises one or more of a tumor-targeting moiety, a cytokine molecule, a stromal modifying moiety, or an immune cell engager.

In some embodiments, the second agent is a multispecific molecule that further comprises one or more of a tumor-targeting moiety, a cytokine molecule, a stromal modifying moiety, or an immune cell engager.

In some embodiments, the first agent comprises the following configuration:

A-,B-[dimerization module]-C,-D,

wherein:
(a) the dimerization module comprises a first immunoglobulin chain constant region and a second immunoglobulin chain constant region;
(b) A and C are linked to the first immunoglobulin chain constant region; and B and D are linked to the second immunoglobulin chain constant region;
(c) A, B, C, and D are independently (i) absent; (ii) the first binder; (iii) the tumor targeting moiety. (iv) the cytokine molecule; (v) stromal modifying moiety; or (vi) the immune cell engager; and wherein at least one, two, or three of A, B, C, and D comprises the first binder.

In some embodiments, the second agent comprises the following configuration:

A-,B-[dimerization module]-C,-D,

wherein:
(a) the dimerization module comprises a first immunoglobulin chain constant region and a second immunoglobulin chain constant region;
(b) A and C are linked to the first immunoglobulin chain constant region; and B and D are linked to the second immunoglobulin chain constant region;
(c) A, B, C, and D are independently (i) absent; (ii) the second binder; (iii) the tumor targeting moiety, (iv) the cytokine molecule; (v) stromal modifying moiety; or (vi) the immune cell engager; and wherein at least one, two, or three of A, B, C, and D comprises the second binder.

In some embodiments, the first immunoglobulin chain constant region comprises the first fragment crystallizable region (Fc region) and the second first immunoglobulin chain constant region comprises the second fragment crystallizable region (Fc region).

In some embodiments, dimerization of the first Fc region and the second Fc region is enhanced by providing an Fc interface of the first Fc region and the second Fc region with one or more of a paired cavity-protuberance, an electrostatic interaction, or a strand-exchange, such that a greater ratio of heteromultimer to homomultimer forms relative to a non-engineered interface.

In some embodiments, the immune cell engager is selected from the group consisting of a T cell engager, an NK cell engager, a B cell engager, a dendritic cell engager, a macrophage cell engager, and any combination thereof.

In some embodiments, the cytokine molecule is selected from the group consisting of interleukin-2 (IL-2) or functional variant thereof, interleukin-7 (IL-7) or functional variant thereof, interleukin-12 (IL-12) or functional variant thereof, interleukin-15 (IL-15) or functional variant thereof, interleukin-18 (IL-18) or functional variant thereof, interleukin-21 (IL-21) or functional variant thereof, interferon gamma or functional variant thereof, and any combination thereof.

In some embodiments, the cytokine molecule comprises interleukin-2 (IL-2) or functional variant thereof.

In some embodiments, the interleukin-2 (IL-2) or functional variant thereof comprises the sequence of SEQ ID NO: 2270 or SEQ ID NO: 2191.

In some embodiments, the cytokine molecule comprises interleukin-15 (IL-15) or functional variant thereof.

In some embodiments, the interleukin-15 (IL-15) or functional variant thereof comprises the sequence of SEQ ID NO: 2170.

In some embodiments, the cytokine molecule further comprises an IL15Ralpha dimerizing domain covalently linked the interleukin-15 (IL-15) or functional variant thereof.

In some embodiments, the IL15Ralpha dimerizing domain comprises an IL-15 receptor alpha sushi domain or functional variant thereof.

In some embodiments, the IL15Ralpha dimerizing domain comprises the sequence of SEQ ID NO: 3472.

In some embodiments, the interleukin-15 (IL-15) or functional variant thereof is covalently linked to the IL15Ralpha dimerizing domain via a link-er.

In some embodiments, the interleukin-15 (IL-15) or functional variant thereof is covalently linked to the IL15Ralpha dimerizing domain via a linker comprising the sequence of SEQ ID NO: 3473.

In some embodiments, the cytokine molecule comprises the sequence of SEQ ID NO: 3474.

In some embodiments, the stromal modifying moiety is selected from the group consisting of a hyaluronidase, a collagenase, a chondroitinase, and a matrix metalloproteinase.

In some embodiments, the tumor-targeting moiety binds to a cancer antigen.

In some embodiments, the tumor-targeting moiety binds to a cancer antigen.

In some embodiments, the cancer antigen is present on a hematological cancer, a solid tumor, a metastatic cancer, a soft tissue tumor, a metastatic lesion, or a combination thereof.

In some embodiments, the cancer antigen is a tumor antigen, a stromal antigen, or a hematological antigen.

In some embodiments, the cancer antigen is selected from the group consisting of BCMA, CD19, CD20, CD22, FcRH5, PDL1, CD47, ganglioside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor. TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, MC1R, β-catenin, BRCA1/2, CDK4, CML66, Fibronectin, p53, Ras, TGF-B receptor, AFP, ETA, MAGE, CA-125, BAGE, GAGE, CDC27, a actinin-4, TRP1/gp75, TRP2, gangliosides, WT1, Epidermal growth factor receptor (EGFR), MART-2, MUC1, MUC2, MUM1, MUM2, MUM3, NA88-1, NPM, OA1, OGT, RCC, RU11, RU12, SAGE, TRG, TSTA, Folate receptor alpha, L1-CAM, CAIX, gpA33, GD3, GM2, VEGFR. Integrins, Carbohydrates. IGF1R, TRAILR1, TRAILR2, RANKL, FAP, TGF-beta, hyaluronic acid, collagen, tenascin C, and tenascin W.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3445, wherein the VH is linked to a heavy chain constant region comprising the sequence of SEQ ID NO: 3649, wherein the heavy chain constant region comprises the first Fc region comprising the sequence of SEQ ID NO: 3447,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 3446, wherein the VL is linked to a light chain constant region comprising the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3448, wherein the VH is linked to a heavy chain constant region comprising the sequence of SEQ ID NO: 3649, wherein the heavy chain constant region comprises the first Fc region comprising the sequence of SEQ ID NO: 3447,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 3449, wherein the VL is linked to a light chain constant region comprising the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3450, wherein the VH is linked to a heavy chain constant region comprising the sequence of SEQ ID NO: 3649, wherein the heavy chain constant region comprises the first Fc region comprising the sequence of SEQ ID NO: 3447,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 3451, wherein the VL is linked to a light chain constant region comprising the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3450, wherein the VH is linked to a heavy chain constant region comprising the sequence of SEQ ID NO: 3649, wherein the heavy chain constant region comprises the first Fc region comprising the sequence of SEQ ID NO: 3447,
(ii) the second polypeptide chain comprises a cytokine molecule, wherein the cytokine molecule is linked to the second Fc region comprising the sequence of SEQ ID NO: 3454 or the sequence of SEQ ID NO: 3501 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 3451, wherein the VL is linked to a light chain constant region comprising the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 1346, wherein the VH is linked to a heavy chain constant region comprising the sequence of SEQ ID NO: 3452 or the sequence of SEQ ID NO: 3504, wherein the heavy chain constant region comprises the first Fc region comprising the sequence of SEQ ID NO: 3453 or the sequence of SEQ ID NO: 3505,
(ii) the second polypeptide chain comprises a cytokine molecule, wherein the cytokine molecule is linked to the second Fc region comprising the sequence of SEQ ID NO: 3454 or the sequence of SEQ ID NO: 3501 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 1349, wherein the VL is linked to a light chain constant region comprising the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 1196, wherein the VH is linked to a heavy chain constant region comprising the sequence of SEQ ID NO: 3649, wherein the heavy chain constant region comprises the first Fc region comprising the sequence of SEQ ID NO: 3447,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 1195, wherein the VL is linked to a light chain constant region comprising the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3455, wherein the VH is linked to a heavy chain constant region comprising the sequence of SEQ ID NO: 3649, wherein the heavy chain constant region comprises the first Fc region comprising the sequence of SEQ ID NO: 3447,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 3456, wherein the VL is linked to a light chain constant region comprising the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3457, wherein the VH is linked to a heavy chain constant region comprising the sequence of SEQ ID NO: 3459, wherein the heavy chain constant region comprises the first Fc region comprising the sequence of SEQ ID NO: 3460,
(ii) the second polypeptide chain comprises a cytokine molecule, wherein the cytokine molecule is linked to the second Fc region comprising the sequence of SEQ ID NO: 3462 via a linker comprising the sequence of SEQ ID NO: 3308, wherein the cytokine molecule comprises IL-2 or functional variant thereof comprising the sequence of SEQ ID NO: 2191, and
(iii) the third polypeptide chain comprises a VL comprising the sequence of SEQ ID NO: 3458, wherein the VL is linked to a light chain constant region comprising the sequence of SEQ ID NO: 3461.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3463, wherein the VH is linked to a heavy chain constant region comprising the sequence of SEQ ID NO: 3459, wherein the heavy chain constant region comprises the first Fc region comprising the sequence of SEQ ID NO: 3460,
(ii) the second polypeptide chain comprises a cytokine molecule, wherein the cytokine molecule is linked to the second Fc region comprising the sequence of SEQ ID NO: 3462 via a linker comprising the sequence of SEQ ID NO: 3308, wherein the cytokine molecule comprises IL-2 or functional variant thereof comprising the sequence of SEQ ID NO: 2191, and
(iii) the third polypeptide chain comprises a VL comprising the sequence of SEQ ID NO: 3464, wherein the VL is linked to a light chain constant region comprising the sequence of SEQ ID NO: 3461.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3465, wherein the VH is linked to a heavy chain constant region comprising the sequence of SEQ ID NO: 3467, wherein the heavy chain constant region comprises the first Fc region comprising the sequence of SEQ ID NO: 3468,
(ii) the second polypeptide chain comprises a cytokine molecule, wherein the cytokine molecule is linked to the second Fc region comprising the sequence of SEQ ID NO: 3469 via a linker comprising the sequence of SEQ ID NO: 3308, wherein the cytokine molecule comprises IL-2 or functional variant thereof comprising the sequence of SEQ ID NO: 2191, and
(iii) the third polypeptide chain comprises a VL comprising the sequence of SEQ ID NO: 3466, wherein the VL is linked to a light chain constant region comprising the sequence of SEQ ID NO: 3461.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3470, wherein the VH is linked to a heavy chain constant region comprising the sequence of SEQ ID NO: 3649, wherein the heavy chain constant region comprises the first Fc region comprising the sequence of SEQ ID NO: 3447,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 3471, wherein the VL is linked to a light chain constant region comprising the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 1346, wherein the VH is linked to a heavy chain constant region comprising the sequence of SEQ ID NO: 3452 or the sequence of SEQ ID NO: 3504, wherein the heavy chain constant region comprises the first Fc region comprising the sequence of SEQ ID NO: 3453 or the sequence of SEQ ID NO: 3505,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-15 or functional variant thereof comprising the sequence of SEQ ID NO: 2170, wherein the IL-15 or functional variant thereof is covalently linked to an IL15Ralpha dimerizing domain comprising the sequence of the SEQ ID NO: 3472 via a linker comprising the sequence of SEQ ID NO: 3473, and
(iii) the third polypeptide chain comprises a VL comprising the sequence of SEQ ID NO: 1349, wherein the VL is linked to a light chain constant region comprising the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 1346, wherein the VH is linked to a heavy chain constant region comprising the sequence of SEQ ID NO: 3452 or the sequence of SEQ ID NO: 3504, wherein the heavy chain constant region comprises the first Fc region comprising the sequence of SEQ ID NO: 3453 or the sequence of SEQ ID NO: 3505,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-15 or functional variant thereof comprising the sequence of SEQ ID NO: 2170, wherein the IL-15 or functional variant thereof is covalently linked to an IL15Ralpha dimerizing domain comprising the sequence of the SEQ ID NO: 3472 via a linker comprising the sequence of SEQ ID NO: 3473, and
(iii) the third polypeptide chain comprises a VL comprising the sequence of SEQ ID NO: 3475, wherein the VL is linked to a light chain constant region comprising the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 1346, wherein the VH is linked to a heavy chain constant region comprising the sequence of SEQ ID NO: 3452 or the sequence of SEQ ID NO: 3504, wherein the heavy chain constant region comprises the first Fc region comprising the sequence of SEQ ID NO: 3453 or the sequence of SEQ ID NO: 3505,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-15 or functional variant thereof comprising the sequence of SEQ ID NO: 2170, wherein the IL-15 or functional variant thereof is covalently linked to an IL15Ralpha dimerizing domain comprising the sequence of the SEQ ID NO: 3472 via a linker comprising the sequence of SEQ ID NO: 3473, and
(iii) the third polypeptide chain comprises a VL comprising the sequence of SEQ ID NO: 3476, wherein the VL is linked to a light chain constant region comprising the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3445, wherein the VH is linked to a heavy chain constant region comprising the sequence of SEQ ID NO: 3649, wherein the heavy chain constant region comprises the first Fc region comprising the sequence of SEQ ID NO: 3447,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 3446, wherein the VL is linked to a light chain constant region comprising the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3448, wherein the VH is linked to a heavy chain constant region comprising the sequence of SEQ ID NO: 3649, wherein the heavy chain constant region comprises the first Fc region comprising the sequence of SEQ ID NO: 3447,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 3449, wherein the VL is linked to a light chain constant region comprising the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3450, wherein the VH is linked to a heavy chain constant region comprising the sequence of SEQ ID NO: 3649, wherein the heavy chain constant region comprises the first Fc region comprising the sequence of SEQ ID NO: 3447,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 3451, wherein the VL is linked to a light chain constant region comprising the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3450, wherein the VH is linked to a heavy chain constant region comprising the sequence of SEQ ID NO: 3649, wherein the heavy chain constant region comprises the first Fc region comprising the sequence of SEQ ID NO: 3447,
(ii) the second polypeptide chain comprises a cytokine molecule, wherein the cytokine molecule is linked to the second Fc region comprising the sequence of SEQ ID NO: 3454 or the sequence of SEQ ID NO: 3501 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 3451, wherein the VL is linked to a light chain constant region comprising the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 1346, wherein the VH is linked to a heavy chain constant region comprising the sequence of SEQ ID NO: 3452 or the sequence of SEQ ID NO: 3504, wherein the heavy chain constant region comprises the first Fc region comprising the sequence of SEQ ID NO: 3453 or the sequence of SEQ ID NO: 3505,
(ii) the second polypeptide chain comprises a cytokine molecule, wherein the cytokine molecule is linked to the second Fc region comprising the sequence of SEQ ID NO: 3454 or the sequence of SEQ ID NO: 3501 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 1349, wherein the VL is linked to a light chain constant region comprising the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 1196, wherein the VH is linked to a heavy chain constant region comprising the sequence of SEQ ID NO: 3649, wherein the heavy chain constant region comprises the first Fc region comprising the sequence of SEQ ID NO: 3447,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 1195, wherein the VL is linked to a light chain constant region comprising the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3455, wherein the VH is linked to a heavy chain constant region comprising the sequence of SEQ ID NO: 3649, wherein the heavy chain constant region comprises the first Fc region comprising the sequence of SEQ ID NO: 3447,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 3456, wherein the VL is linked to a light chain constant region comprising the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3457, wherein the VH is linked to a heavy chain constant region comprising the sequence of SEQ ID NO: 3459, wherein the heavy chain constant region comprises the first Fc region comprising the sequence of SEQ ID NO: 3460,
(ii) the second polypeptide chain comprises a cytokine molecule, wherein the cytokine molecule is linked to the second Fc region comprising the sequence of SEQ ID NO: 3462 via a linker comprising the sequence of SEQ ID NO: 3308, wherein the cytokine molecule comprises IL-2 or functional variant thereof comprising the sequence of SEQ ID NO: 2191, and
(iii) the third polypeptide chain comprises a VL comprising the sequence of SEQ ID NO: 3458, wherein the VL is linked to a light chain constant region comprising the sequence of SEQ ID NO: 3461.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3463, wherein the VH is linked to a heavy chain constant region comprising the sequence of SEQ ID NO: 3459, wherein the heavy chain constant region comprises the first Fc region comprising the sequence of SEQ ID NO: 3460,
(ii) the second polypeptide chain comprises a cytokine molecule, wherein the cytokine molecule is linked to the second Fc region comprising the sequence of SEQ ID NO: 3462 via a linker comprising the sequence of SEQ ID NO: 3308, wherein the cytokine molecule comprises IL-2 or functional variant thereof comprising the sequence of SEQ ID NO: 2191, and
(iii) the third polypeptide chain comprises a VL comprising the sequence of SEQ ID NO: 3464, wherein the VL is linked to a light chain constant region comprising the sequence of SEQ ID NO: 3461.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3465, wherein the VH is linked to a heavy chain constant region comprising the sequence of SEQ ID NO: 3467, wherein the heavy chain constant region comprises the first Fc region comprising the sequence of SEQ ID NO: 3468,
(ii) the second polypeptide chain comprises a cytokine molecule, wherein the cytokine molecule is linked to the second Fc region comprising the sequence of SEQ ID NO: 3469 via a linker comprising the sequence of SEQ ID NO: 3308, wherein the cytokine molecule comprises IL-2 or functional variant thereof comprising the sequence of SEQ ID NO: 2191, and
(iii) the third polypeptide chain comprises a VL comprising the sequence of SEQ ID NO: 3466, wherein the VL is linked to a light chain constant region comprising the sequence of SEQ ID NO: 3461.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 3470, wherein the VH is linked to a heavy chain constant region comprising the sequence of SEQ ID NO: 3649, wherein the heavy chain constant region comprises the first Fc region comprising the sequence of SEQ ID NO: 3447,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-2 or functional variant thereof comprising the sequence of SEQ ID NO: 2270, and
(iii) the third polypeptide chain comprises a VL comprising the sequence of SEQ ID NO: 3471, wherein the VL is linked to a light chain constant region comprising the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 1346, wherein the VH is linked to a heavy chain constant region comprising the sequence of SEQ ID NO: 3452 or the sequence of SEQ ID NO: 3504, wherein the heavy chain constant region comprises the first Fc region comprising the sequence of SEQ ID NO: 3453 or the sequence of SEQ ID NO: 3505,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-15 or functional variant thereof comprising the sequence of SEQ ID NO: 2170, wherein the IL-15 or functional variant thereof is covalently linked to an IL15Ralpha dimerizing domain comprising the sequence of the SEQ ID NO: 3472 via a linker comprising the sequence of SEQ ID NO: 3473, and
(iii) the third polypeptide chain comprises a VL comprising the sequence of SEQ ID NO: 1349, wherein the VL is linked to a light chain constant region comprising the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 1346, wherein the VH is linked to a heavy chain constant region comprising the sequence of SEQ ID NO: 3452 or the sequence of SEQ ID NO: 3504, wherein the heavy chain constant region comprises the first Fc region comprising the sequence of SEQ ID NO: 3453 or the sequence of SEQ ID NO: 3505,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-15 or functional variant thereof comprising the sequence of SEQ ID NO: 2170, wherein the IL-15 or functional variant thereof is covalently linked to an IL15Ralpha dimerizing domain comprising the sequence of the SEQ ID NO: 3472 via a linker comprising the sequence of SEQ ID NO: 3473, and
(iii) the third polypeptide chain comprises a VL comprising the sequence of SEQ ID NO: 3475, wherein the VL is linked to a light chain constant region comprising the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising the sequence of SEQ ID NO: 1346, wherein the VH is linked to a heavy chain constant region comprising the sequence of SEQ ID NO: 3452 or the sequence of SEQ ID NO: 3504, wherein the heavy chain constant region comprises the first Fc region comprising the sequence of SEQ ID NO: 3453 or the sequence of SEQ ID NO: 3505,
(ii) the second polypeptide chain comprises a cytokine molecule, wherein the cytokine molecule is linked to the second Fc region comprising the sequence of SEQ ID NO: 3648 via a linker comprising the sequence of SEQ ID NO: 3308, wherein the cytokine molecule comprises IL-15 or functional variant thereof comprising the sequence of SEQ ID NO: 2170, wherein the IL-15 or functional variant thereof is covalently linked to an IL15Ralpha dimerizing domain comprising the sequence of the SEQ ID NO: 3472 via a linker comprising the sequence of SEQ ID NO: 3473, and
(iii) the third polypeptide chain comprises a VL comprising the sequence of SEQ ID NO: 3476, wherein the VL is linked to a light chain constant region comprising the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3338;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3339.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3318;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3319.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3328;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3329.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3328;
(ii) the second polypeptide chain comprises a sequence having at least 75% sequence identity to the sequence of SEQ ID NO: 3350 or the sequence of SEQ ID NO: 3500; and
(iii) the third polypeptide chain comprises a sequence having at least 75% sequence identity to the sequence of SEQ ID NO: 3329.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3348 or the sequence of SEQ ID NO: 3502;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3349.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3358;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3359.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3368;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3369.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3378;
(ii) the second polypeptide chain comprises a sequence having at least 75% sequence identity to the sequence of SEQ ID NO: 3380; and
(iii) the third polypeptide chain comprises a sequence having at least 75% sequence identity to the sequence of SEQ ID NO: 3379.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3388;
(ii) the second polypeptide chain comprises a sequence having at least 75% sequence identity to the sequence of SEQ ID NO: 3390; and
(iii) the third polypeptide chain comprises a sequence having at least 75% sequence identity to the sequence of SEQ ID NO: 3389.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3398;
(ii) the second polypeptide chain comprises a sequence having at least 75% sequence identity to the sequence of SEQ ID NO: 3400; and
(iii) the third polypeptide chain comprises a sequence having at least 75% sequence identity to the sequence of SEQ ID NO: 3399.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3408;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3409.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3348 or the sequence of SEQ ID NO: 3502;
(ii) the second polypeptide chain comprises a sequence having at least 75% sequence identity to the sequence of SEQ ID NO: 3420; and
(iii) the third polypeptide chain comprises a sequence having at least 75% sequence identity to the sequence of SEQ ID NO: 3349.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3348 or the sequence of SEQ ID NO: 3502;
(ii) the second polypeptide chain comprises a sequence having at least 75% sequence identity to the sequence of SEQ ID NO: 3420; and
(iii) the third polypeptide chain comprises a sequence having at least 75% sequence identity to the sequence of SEQ ID NO: 3428.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3348 or the sequence of SEQ ID NO: 3502;
(ii) the second polypeptide chain comprises a sequence having at least 75% sequence identity to the sequence of SEQ ID NO: 3420; and
(iii) the third polypeptide chain comprises a sequence having at least 75% sequence identity to the sequence of SEQ ID NO: 3437.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3338;
(ii) the second polypeptide chain comprises the sequence of SEQ ID NO: 3340 or the sequence of SEQ ID NO: 3503; and
(iii) the third polypeptide chain comprises the sequence of SEQ ID NO: 3339.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3318;
(ii) the second polypeptide chain comprises the sequence of SEQ ID NO: 3340 or the sequence of SEQ ID NO: 3503; and
(iii) the third polypeptide chain comprises the sequence of SEQ ID NO: 3319.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3328;
(ii) the second polypeptide chain comprises the sequence of SEQ ID NO: 3340 or the sequence of SEQ ID NO: 3503; and
(iii) the third polypeptide chain comprises the sequence of SEQ ID NO: 3329.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3328;
(ii) the second polypeptide chain comprises the sequence of SEQ ID NO: 3350 or the sequence of SEQ ID NO: 3500; and
(iii) the third polypeptide chain comprises the sequence of SEQ ID NO: 3329.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3348 or the sequence of SEQ ID NO: 3502;
(ii) the second polypeptide chain comprises the sequence of SEQ ID NO: 3340 or the sequence of SEQ ID NO: 3503; and
(iii) the third polypeptide chain comprises the sequence of SEQ ID NO: 3349.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3358;
(ii) the second polypeptide chain comprises the sequence of SEQ ID NO: 3340 or the sequence of SEQ ID NO: 3503; and
(iii) the third polypeptide chain comprises the sequence of SEQ ID NO: 3359.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3368;
(ii) the second polypeptide chain comprises the sequence of SEQ ID NO: 3340 or the sequence of SEQ ID NO: 3503; and
(iii) the third polypeptide chain comprises the sequence of SEQ ID NO: 3369.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3378;
(ii) the second polypeptide chain comprises the sequence of SEQ ID NO: 3380; and
(iii) the third polypeptide chain comprises the sequence of SEQ ID NO: 3379.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3388;
(ii) the second polypeptide chain comprises the sequence of SEQ ID NO: 3390; and
(iii) the third polypeptide chain comprises the sequence of SEQ ID NO: 3389.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3398;
(ii) the second polypeptide chain comprises the sequence of SEQ ID NO: 3400; and
(iii) the third polypeptide chain comprises the sequence of SEQ ID NO: 3399.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3408;
(ii) the second polypeptide chain comprises the sequence of SEQ ID NO: 3340 or the sequence of SEQ ID NO: 3503; and
(iii) the third polypeptide chain comprises the sequence of SEQ ID NO: 3409.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3348 or the sequence of SEQ ID NO: 3502;
(ii) the second polypeptide chain comprises the sequence of SEQ ID NO: 3420; and
(iii) the third polypeptide chain comprises the sequence of SEQ ID NO: 3349.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3348 or the sequence of SEQ ID NO: 3502;
(ii) the second polypeptide chain comprises the sequence of SEQ ID NO: 3420; and
(iii) the third polypeptide chain comprises the sequence of SEQ ID NO: 3428.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3348 or the sequence of SEQ ID NO: 3502;
(ii) the second polypeptide chain comprises the sequence of SEQ ID NO: 3420; and
(iii) the third polypeptide chain comprises the sequence of SEQ ID NO: 3437.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3338;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3339.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3318;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3319.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3328;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3329.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3328;
(ii) the second polypeptide chain comprises a sequence having at least 75% sequence identity to the sequence of SEQ ID NO: 3350 or the sequence of SEQ ID NO: 3500; and
(iii) the third polypeptide chain comprises a sequence having at least 75% sequence identity to the sequence of SEQ ID NO: 3329.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3348 or the sequence of SEQ ID NO: 3502;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3349.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3358;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3359.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3368;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3369.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3378;
(ii) the second polypeptide chain comprises a sequence having at least 75% sequence identity to the sequence of SEQ ID NO: 3380; and
(iii) the third polypeptide chain comprises a sequence having at least 75% sequence identity to the sequence of SEQ ID NO: 3379.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3388;
(ii) the second polypeptide chain comprises a sequence having at least 75% sequence identity to the sequence of SEQ ID NO: 3390; and
(iii) the third polypeptide chain comprises a sequence having at least 75% sequence identity to the sequence of SEQ ID NO: 3389.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3398;
(ii) the second polypeptide chain comprises a sequence having at least 75% sequence identity to the sequence of SEQ ID NO: 3400; and
(iii) the third polypeptide chain comprises a sequence having at least 75% sequence identity to the sequence of SEQ ID NO: 3399.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3408;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3409.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3348 or the sequence of SEQ ID NO: 3502;
(ii) the second polypeptide chain comprises a sequence having at least 75% sequence identity to the sequence of SEQ ID NO: 3420; and
(iii) the third polypeptide chain comprises a sequence having at least 75% sequence identity to the sequence of SEQ ID NO: 3349.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3348 or the sequence of SEQ ID NO: 3502;
(ii) the second polypeptide chain comprises a sequence having at least 75% sequence identity to the sequence of SEQ ID NO: 3420; and
(iii) the third polypeptide chain comprises a sequence having at least 75% sequence identity to the sequence of SEQ ID NO: 3428.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having at least 75% sequence identity to the sequence of SEQ ID NO: 3348 or the sequence of SEQ ID NO: 3502;
(ii) the second polypeptide chain comprises a sequence having at least 75% sequence identity to the sequence of SEQ ID NO: 3420; and
(iii) the third polypeptide chain comprises a sequence having at least 75% sequence identity to the sequence of SEQ ID NO: 3437.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3338;
(ii) the second polypeptide chain comprises the sequence of SEQ ID NO: 3340 or the sequence of SEQ ID NO: 3503; and
(iii) the third polypeptide chain comprises the sequence of SEQ ID NO: 3339.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3318;
(ii) the second polypeptide chain comprises the sequence of SEQ ID NO: 3340 or the sequence of SEQ ID NO: 3503; and
(iii) the third polypeptide chain comprises the sequence of SEQ ID NO: 3319.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3328;
(ii) the second polypeptide chain comprises the sequence of SEQ ID NO: 3340 or the sequence of SEQ ID NO: 3503; and
(iii) the third polypeptide chain comprises the sequence of SEQ ID NO: 3329.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3328;
(ii) the second polypeptide chain comprises the sequence of SEQ ID NO: 3350 or the sequence of SEQ ID NO: 3500; and
(iii) the third polypeptide chain comprises the sequence of SEQ ID NO: 3329.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3348 or the sequence of SEQ ID NO: 3502;
(ii) the second polypeptide chain comprises the sequence of SEQ ID NO: 3340 or the sequence of SEQ ID NO: 3503; and
(iii) the third polypeptide chain comprises the sequence of SEQ ID NO: 3349.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3358;
(ii) the second polypeptide chain comprises the sequence of SEQ ID NO: 3340 or the sequence of SEQ ID NO: 3503; and
(iii) the third polypeptide chain comprises the sequence of SEQ ID NO: 3359.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3368;
(ii) the second polypeptide chain comprises the sequence of SEQ ID NO: 3340 or the sequence of SEQ ID NO: 3503; and
(iii) the third polypeptide chain comprises the sequence of SEQ ID NO: 3369.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3378;
(ii) the second polypeptide chain comprises the sequence of SEQ ID NO: 3380; and
(iii) the third polypeptide chain comprises the sequence of SEQ ID NO: 3379.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3388;
(ii) the second polypeptide chain comprises the sequence of SEQ ID NO: 3390; and
(iii) the third polypeptide chain comprises the sequence of SEQ ID NO: 3389.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3398;
(ii) the second polypeptide chain comprises the sequence of SEQ ID NO: 3400; and
(iii) the third polypeptide chain comprises the sequence of SEQ ID NO: 3399.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3408;
(ii) the second polypeptide chain comprises the sequence of SEQ ID NO: 3340 or the sequence of SEQ ID NO: 3503; and
(iii) the third polypeptide chain comprises the sequence of SEQ ID NO: 3409.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3348 or the sequence of SEQ ID NO: 3502;
(ii) the second polypeptide chain comprises the sequence of SEQ ID NO: 3420; and
(iii) the third polypeptide chain comprises the sequence of SEQ ID NO: 3349.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3348 or the sequence of SEQ ID NO: 3502;
(ii) the second polypeptide chain comprises the sequence of SEQ ID NO: 3420; and
(iii) the third polypeptide chain comprises the sequence of SEQ ID NO: 3428.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises the sequence of SEQ ID NO: 3348 or the sequence of SEQ ID NO: 3502;
(ii) the second polypeptide chain comprises the sequence of SEQ ID NO: 3420; and
(iii) the third polypeptide chain comprises the sequence of SEQ ID NO: 3437.

In some embodiments, the disease or condition is cancer.

In some embodiments, the cancer is a solid tumor.

In some embodiments, the solid tumor is melanoma, pancreatic cancer, breast cancer, colorectal cancer, lung cancer, skin cancer, ovarian cancer, or liver cancer.

In some embodiments, the solid tumor is selected from the group consisting of high mutational burden (TMB-H), microsatellite instability/DNA mismatch repair (MSI-H/dMMR), virally associated tumor, metastatic triple-negative breast cancer (mTNBC), relapsed and refractory epithelial ovarian cancer, metastatic castration-resistant prostate cancer (mCRPC), K-Ras wild type CRC, K-Ras mutant CRC, and primary stage IV or recurrent non-small cell lung cancer (NSCLC).

In some embodiments, the virally associated tumor comprises Merkel cell carcinoma, cervical cancer, oropharyngeal cancer, anal cancer, penile cancer, vaginal cancer, or vulvar cancer.

In some embodiments, the cancer is a hematological cancer.

In some embodiments, the hematological cancer is a B-cell or T cell malignancy.

In some embodiments, the B-cell or T cell malignancy is Hodgkin's lymphoma, Non-Hodgkin's lymphoma, acute myeloid leukemia (AML), chronic myeloid leukemia, myelodysplastic syndrome, multiple myeloma, or acute lymphocytic leukemia.

In some embodiments, the Non-Hodgkin's lymphoma is B cell lymphoma, diffuse large B cell lymphoma, follicular lymphoma, chronic lymphocytic leukemia, mantle cell lymphoma, marginal zone B-cell lymphoma, Burkitt lymphoma, lymphoplasmacytic lymphoma, or hairy cell leukemia.

In some embodiments, the administering comprises administering a first pharmaceutical composition comprising the first agent, and a second pharmaceutical composition comprising the second agent, wherein the first pharmaceutical composition and the second pharmaceutical composition independently further comprises a pharmaceutically acceptable excipient, carrier or diluent.

In another aspect, provided herein is, inter alia, a kit for the method as provided herein or the combination therapy as provided herein comprising the first agent, and the second agent.

In another aspect, provided herein is, inter alia, a kit for the method as provided herein or the combination therapy as provided herein comprising a first pharmaceutical composition comprising the first agent, and a second pharmaceutical composition comprising the second agent, wherein the first pharmaceutical composition and the second pharmaceutical composition independently further comprises a pharmaceutically acceptable excipient, carrier or diluent.

In some embodiments, the kit comprises the first agent in an amount sufficient for the administration to the subject at a dose of from about 0.001 mg/kg to about 10 mg/kg.

In some embodiments, the kit comprises the first agent in an amount sufficient for the administration to the subject at a dose of about 0.3 mg/kg or about 1 mg/kg.

In some embodiments, the kit comprises the second agent in an amount sufficient for the administration to the subject at a dose of from about 0.001 mg/kg to about 10 mg/kg.

In some embodiments, the kit comprises the second agent in an amount sufficient for the administration to the subject at a dose of about 0.3 mg/kg or about 1 mg/kg.

In another aspect, provided herein is, inter alia, a dose regimen for the combination therapy as provided herein or the combination therapy as provided herein.

In some embodiments, the first agent and the second agent are not concomitantly administered to the subject.

In some embodiments, the administration of the first agent comprises one or more doses.

In some embodiments, the administration of the first agent comprises two or more doses.

In some embodiments, the administration of the first agent comprises one dose per week.

In some embodiments, the administration of the first agent comprises two or more doses per week.

In some embodiments, the first agent is administered to the subject once every two weeks.

In some embodiments, the first agent is administered to the subject once every three weeks.

In some embodiments, the first agent is administered to the subject once every week for at least 1, 2, 3, or 4 weeks, at least 1, 2, 3, 4, 5, 6, 7, 8, 9, 10, 11 or 12 months, or at least 1, 2, or 3 years.

In some embodiments, the administration of the first agent comprises two or more doses per week for at least 1, 2, 3, or 4 weeks, at least 1, 2, 3, 4, 5, 6, 7, 8, 9, 10, 11 or 12 months, or at least 1, 2, or 3 years.

In some embodiments, the first agent is administered to the subject once every two weeks for at least 1, 2, 3, or 4 weeks, at least 1, 2, 3, 4, 5, 6, 7, 8, 9, 10, 11 or 12 months, or at least 1, 2, or 3 years.

In some embodiments, the first agent is administered to the subject once every three weeks for at least 1, 2, 3, or 4 weeks, at least 1, 2, 3, 4, 5, 6, 7, 8, 9, 10, 11 or 12 months, or at least 1, 2, or 3 years.

In some embodiments, the administration of the second agent comprises one or more doses.

In some embodiments, the administration of the second agent comprises two or more doses.

In some embodiments, the administration of the second agent comprises one dose per week.

In some embodiments, the administration of the second agent comprises two or more doses per week.

In some embodiments, the second agent is administered to the subject once every two weeks.

In some embodiments, the second agent is administered to the subject once every three weeks.

In some embodiments, the second agent is administered to the subject once every week for at least 1, 2, 3, or 4 weeks, at least 1, 2, 3, 4, 5, 6, 7, 8, 9, 10, 11 or 12 months, or at least 1, 2, or 3 years.

In some embodiments, the administration of the second agent comprises two or more doses per week for at least 1, 2, 3, or 4 weeks, at least 1, 2, 3, 4, 5, 6, 7, 8, 9, 10, 11 or 12 months, or at least 1, 2, or 3 years.

In some embodiments, the second agent is administered to the subject once every two weeks for at least 1, 2, 3, or 4 weeks, at least 1, 2, 3, 4, 5, 6, 7, 8, 9, 10, 11 or 12 months, or at least 1, 2, or 3 years.

In some embodiments, the second agent is administered to the subject once every three weeks for at least 1, 2, 3, or 4 weeks, at least 1, 2, 3, 4, 5, 6, 7, 8, 9, 10, 11 or 12 months, or at least 1, 2, or 3 years.

In some embodiments, the administration of the first agent and the administration of the second agent is separated by one week.

In some embodiments, the administration of the first agent and the administration of the second agent is separated by two or more weeks.

In some embodiments, the administration of the first agent and the administration of the second agent is separated by 1 day, 2 days, 3 days, 4 days, 5 days, 6 days, 1 week, 2 weeks, 3 weeks, 4 weeks, 1 month, 2 months, 3 months, 4 months, 5 months, 6 months, 7 months, 8 months, 9 months, 10 months, 11 months, or 12 months.

In some embodiments, the administration of the first agent and the second agent is repeated twice or more.

In some embodiments, the dose regimen comprises an administration of the first agent once every week for two weeks followed by an administration of the second agent once a week for two weeks, wherein the second agent is administered to the subject one week after the last administration of the first agent.

In some embodiments, the dose regimen is repeated twice or more.

In some embodiments, the dose regimen is repeated at the interval of one week.

In some embodiments, the dose regimen is repeated at the interval of 1 day, 2 days, 3 days, 4 days, 5 days, 6 days, 1 week, 2 weeks, 3 weeks, 4 weeks, 1 month, 2 months, 3 months, 4 months, 5 months, 6 months, 7 months, 8 months, 9 months, 10 months, 11 months, or 12 months.

In some embodiments, the first agent and the second agent are administered to the subject in a same amount.

In some embodiments, the first agent and the second agent are administered to the subject in different amounts.

In some embodiments, the administration of the first agent comprises two or more doses, and each dose comprises a same amount of the first agent.

In some embodiments, the administration of the first agent comprises two or more doses, and each dose comprises different amounts of the first agent.

In some embodiments, the administration of the second agent comprises two or more doses, and each dose comprises a same amount of the second agent.

In some embodiments, the administration of the second agent comprises two or more doses, and each dose comprises different amounts of the second agent.

In some embodiments, the first agent is administered to the subject at a dose of from about 0.001 mg/kg to about 10 mg/kg.

In some embodiments, the second agent is administered to the subject at a dose of from about 0.001 mg/kg to about 10 mg/kg.

In some embodiments, the first agent is administered to the subject at a dose of about 0.3 mg/kg or about 1 mg/kg.

In some embodiments, the second agent is administered to the subject at a dose of about 0.3 mg/kg or about 1 mg/kg.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts exemplary embodiments of sequentially administration regimen schedule of anti-TCRβV antibody molecule combination therapy described herein in tumor-bearing mice.

FIG. 2 depicts tumor volumes during the administration regimen of anti-TCRβV antibody molecule combination therapy in various treatment groups (e.g., murine TCRβV-13 antibody followed by murine TCRβV-17 antibody.

FIG. 3 depicts the baseline frequency of various exemplary TCRβVs (or TCR Vβs), i.e., mouse TCR Vβ13 (“TCR Vβ13”), mouse TCR Vβ15 (“TCR Vβ15”), mouse TCR Vβ17(“TCR Vβ17”), and mouse TCR Vβ29 (“TCR Vβ29”) in balb/c and c57bl/6 albino mice.

FIG. 4A and FIG. 4B depict the anti-tumor effects of the exemplary agents that comprises a binder that binds to a target molecule, i.e., a T cell receptor beta variable region (TCRβV) binder, and a cytokine molecule, as provided herein, in a mouse tumor model bearing the murine CT26 colon carcinoma cells. FIG. 4A shows that administration of the exemplary agents that comprises a binder that binds to a target molecule, i.e., a T cell receptor beta variable region (TCRβV) binder, and a cytokine molecule, as provided herein, delayed tumor growth or reduced the tumor volume in a mouse tumor model bearing the murine CT26 colon carcinoma cells, relative to administration of vehicle control PBS (phosphate-buffered saline). FIG. 4B shows the percentage of tumor growth inhibition (TGI %) at 24 day post grafting of the CT26 colon carcinoma cells. The agent “m1302” (also referred as “mSTAR1302” or “Vβ13×IL-2”) comprises a binder that binds to mouse TCR Vβ13, and IL-2, and is a mouse surrogate for an agent that comprises a binder that binds to human TCR Vβ06, and IL-2 (referred as “0602”, “STAR0602”, or “Vβ06×IL-2”). The agent “m1502” (also referred as “mSTAR1502” or “Vβ15×IL-2”) comprises a binder that binds to mouse TCR Vβ15, and IL-2, and is a mouse surrogate for an agent that comprises a binder that binds to human TCR Vβ12, and IL-2 (referred as “1202”, “STAR1202”, or “Vβ12×IL-2”). The agent “m1702” (also referred as “mSTAR1702” or “Vβ17×IL-2”) comprises a binder that binds to mouse TCR Vβ17, and IL-2, and is a mouse surrogate for an agent that comprises a binder that binds to human TCR Vβ15, and IL-2 (referred as “1502”, “STAR1502”, or “Vβ15×IL-2”). The agent “m2902” (also referred as “mSTAR2902” or “Vβ29×IL-2”) comprises a binder that binds to mouse TCR Vβ29, and IL-2, and is a mouse surrogate for an agent that comprises a binder that binds to human TCR Vβ28, and IL-2 (referred as “2802”, “STAR2802”, or “Vβ28×IL-2”). “0.3” indicates administration of the agent at the dose of 0.3 mg/kg, and “1.0” indicates administration of the agent at the dose of 1.0 mg/kg. The agents were administered once weekly (QW).

FIG. 5A and FIG. 5B depict the anti-tumor effects of the exemplary agents that comprises a binder that binds to a target molecule, i.e., a T cell receptor beta variable region (TCRβV) binder, and a cytokine molecule, as provided herein, in a mouse tumor model bearing the murine B16-F10 melanoma cells. FIG. 5A shows that administration of the exemplary agents that comprises a binder that binds to a target molecule, i.e., a T cell receptor beta variable region (TCRβV) binder, and a cytokine molecule, as provided herein, delayed tumor growth or reduced the tumor volume in a mouse tumor model bearing the murine B16-F10 melanoma cells, relative to administration of vehicle control PBS (phosphate-buffered saline). FIG. 5B shows the percentage of tumor growth inhibition (TGI %) at 21 day post grafting of the B16-F10 melanoma cells. The agent “m1302” (also referred as “mSTAR1302” or “Vβ13×IL-2”) comprises a binder that binds to mouse TCR Vβ13, and IL-2, and is a mouse surrogate for an agent that comprises a binder that binds to human TCR Vβ06, and IL-2 (referred as “0602”, “STAR0602”, or “Vβ06×IL-2”). The agent “m1502” (also referred as “mSTAR1502” or “Vβ15×IL-2”) comprises a binder that binds to mouse TCR Vβ15, and IL-2, and is a mouse surrogate for an agent that comprises a binder that binds to human TCR Vβ12, and IL-2 (referred as “1202”, “STAR1202”, or “V12×IL-2”). The agent “m1702” (also referred as “mSTAR1702” or “Vβ17×IL-2”) comprises a binder that binds to mouse TCR Vβ17, and IL-2, and is a mouse surrogate for an agent that comprises a binder that binds to human TCR Vβ15, and IL-2 (referred as “1502”, “STAR1502”, or “Vβ15×IL-2”). The agent “m2902” (also referred as “mSTAR2902” or “Vβ29×IL-2”) comprises a binder that binds to mouse TCR Vβ29, and IL-2, and is a mouse surrogate for an agent that comprises a binder that binds to human TCR Vβ28, and IL-2 (referred as “2802”, “STAR2802”, or “Vβ28×IL-2”). “0.3” or “0.3 mpk” indicates administration of the agent at the dose of 0.3 mg/kg, and “1.0” or “1.0 mpk” indicates administration of the agent at the dose of 1.0 mg/kg. The agents were administered once weekly (QW).

FIGS. 6A-6C depict the anti-tumor effects of the exemplary agents that comprises a binder that binds to a target molecule, i.e., a T cell receptor beta variable region (TCRβV) binder, and a cytokine molecule, as provided herein, in a mouse MC38 tumor model of colorectal carcinoma. FIG. 6A shows that administration of m1502, an exemplary agent that comprises a binder that binds to a target molecule, i.e., a T cell receptor beta variable region (TCRβV) binder, and a cytokine molecule, as provided herein, improved the survival of the mouse MC38 tumor model of colorectal carcinoma, relative to administration of vehicle control PBS (phosphate-buffered saline). The agent “m1502” (also referred as “mSTAR1502” or “Vβ15×IL-2”) comprises a binder that binds to mouse TCR Vβ15, and IL-2, and is a mouse surrogate for an agent that comprises a binder that binds to human TCR Vβ12, and IL-2 (referred as “1202”, “STAR1202”, or “Vβ12×IL-2”). “0.3 mg/kg” indicates administration of the agent at the dose of 0.3 mg/kg, and “1.0 mg/kg” indicates administration of the agent at the dose of 1.0 mg/kg. FIG. 6B shows that administration of m1702, an exemplary agent that comprises a binder that binds to a target molecule, i.e., a T cell receptor beta variable region (TCRβV) binder, and a cytokine molecule, as provided herein, improved the survival of the mouse MC38 tumor model of colorectal carcinoma, relative to administration of vehicle control PBS (phosphate-buffered saline). The agent “m1702” (also referred as “mSTAR1702” or “Vβ17×IL-2”) comprises a binder that binds to mouse TCR Vβ17, and IL-2, and is a mouse surrogate for an agent that comprises a binder that binds to human TCR Vβ15, and IL-2 (referred as “1502”, “STAR1502”, or “Vβ15×IL-2”). “0.3 mg/kg” indicates administration of the agent at the dose of 0.3 mg/kg, and “1.0 mg/kg” indicates administration of the agent at the dose of 1.0 mg/kg. FIG. 6C shows that administration of m2902, an exemplary agent that comprises a binder that binds to a target molecule, i.e., a T cell receptor beta variable region (TCRβV) binder, and a cytokine molecule, as provided herein, improved the survival of the mouse MC38 tumor model of colorectal carcinoma, relative to administration of vehicle control PBS (phosphate-buffered saline). The agent “m2902” (also referred as “mSTAR2902” or “Vβ29×IL-2”) comprises a binder that binds to mouse TCR Vβ29, and IL-2, and is a mouse surrogate for an agent that comprises a binder that binds to human TCR Vβ28, and IL-2 (referred as “2802”, “STAR2802”, or “Vβ28×IL-2”). “0.3 mg/kg” indicates administration of the agent at the dose of 0.3 mg/kg, and “1.0 mg/kg” indicates administration of the agent at the dose of 1.0 mg/kg. The agents were administered once weekly (QW).

FIG. 7 shows that sequential administration of mSTAR1302 (“Vβ13×IL-2”), an exemplary first agent that comprises a first binder that binds to a first target molecule, i.e., a binder that binds to mouse TCRVβ13, and a cytokine molecule, i.e., IL-2, as provided herein, followed by administration of mSTAR1702 (“Vβ17×IL-2”), an exemplary second agent that comprises a second binder that binds to a second target molecule, i.e., a binder that binds to mouse TCRVβ17, and a cytokine molecule, i.e., IL-2, as provided herein, at the dose of 1.0 mg/kg once weekly (QW) to a mouse MC38 tumor model of colorectal carcinoma reversed tumor stasis and drove complete responses to the treatment. For example, the graph in FIG. 7 shows that sequential administration of mSTAR1302 (“Vβ13×IL-2”), an exemplary first agent that comprises a first binder that binds to a first target molecule, i.e., a binder that binds to mouse TCRVβ13, and a cytokine molecule, i.e., IL-2, as provided herein, followed by administration of mSTAR1702 (“Vβ17×IL-2”), an exemplary second agent that comprises a second binder that binds to a second target molecule, i.e., a binder that binds to mouse TCRVβ17, and a cytokine molecule, i.e., IL-2, as provided herein, at the dose of 1.0 mg/kg once weekly (QW) to a mouse MC38 tumor model of colorectal carcinoma dramatically reduced the tumor volume, relative to administration of vehicle control PBS (phosphate-buffered saline), administration of mSTAR1302 (“Vβ13×IL-2”) alone, or administration of mSTAR1702 (“Vβ17×IL-2”) alone. For example, on 33 days post implant of MC38 colorectal carcinoma cells, the tumor almost completely disappeared by sequential administration of mSTAR1302 (“Vβ13×IL-2”) followed by administration of mSTAR1702 (“Vβ17×IL-2”). The agent “mSTAR1302” (also referred as “m1302” or “Vβ13×IL-2”) comprises a binder that binds to mouse TCR Vβ13, and IL-2, and is a mouse surrogate for an agent that comprises a binder that binds to human TCR Vβ06, and IL-2 (referred as “0602”, “STAR0602”, or “Vβ06×IL-2”). The agent “mSTAR1702” (also referred as “m1702” or “Vβ17×IL-2”) comprises a binder that binds to mouse TCR Vβ17, and IL-2, and is a mouse surrogate for an agent that comprises a binder that binds to human TCR Vβ15, and IL-2 (referred as “1502”, “STAR1502”, or “Vβ15×IL-2”). The agent “Vβ29×IL-2” (also referred as “m2902” or “mSTAR2902”) comprises a binder that binds to mouse TCR Vβ29, and IL-2, and is a mouse surrogate for an agent that comprises a binder that binds to human TCR Vβ28, and IL-2 (referred as “2802”, “STAR2802”, or “Vβ28×IL-2”).

FIG. 8 shows the reversed tumor statis and complete responses to the treatment observed in 10 individual mice by sequential administration of mSTAR1302 (“Vβ13×IL-2”), an exemplary first agent that comprises a first binder that binds to a first target molecule, i.e., a binder that binds to mouse TCRVβ13, and a cytokine molecule, i.e., IL-2, as provided herein, followed by administration of mSTAR1702 (“Vβ17×IL-2”), an exemplary second agent that comprises a second binder that binds to a second target molecule, i.e., a binder that binds to mouse TCRVβ17, and a cytokine molecule, i.e., IL-2, as provided herein, at the dose of 1.0 mg/kg once weekly (QW) to a mouse MC38 tumor model of colorectal carcinoma. Nine out of 10 mice exhibited tumor-free status by sequential administration of mSTAR1302 (“Vβ13×IL-2”) followed by administration of mSTAR1702 (“Vβ17×IL-2”). In some embodiments, addition of mSTAR1702 appeared to rescue anti-tumor activity in mSTAR1302-resistant tumors, as indicated by the upper arrow. In some embodiments, addition of mSTAR1702 also appeared to drive furth anti-tumor activity in mice with stable disease (SD), as indicated by the lower arrow.

FIGS. 9A and 9B show that concurrent combination of mSTAR1302 (“Vβ13×IL-2”), an exemplary first agent that comprises a first binder that binds to a first target molecule, i.e., a binder that binds to mouse TCRVβ13, and a cytokine molecule, i.e., IL-2, as provided herein, and Vβ17×IL-2 (mSTAR1702), an exemplary second agent that comprises a second binder that binds to a second target molecule, i.e., a binder that binds to mouse TCRVβ17, and a cytokine molecule, i.e., IL-2, as provided herein, did not lead to enhanced anti-tumor activity in the mouse MC38 tumor model of colorectal carcinoma. FIG. 9A shows that concurrent administration of an exemplary first agent, mSTAR1302 (“Vβ13×IL-2”), that comprises a first binder that binds to a first target molecule, i.e., a binder that binds to mouse TCRVβ13, and a cytokine molecule, i.e., IL-2, as provided herein, and an exemplary second agent, Vβ17×IL-2 (mSTAR1702), that comprises a second binder that binds to a second target molecule, i.e., a binder that binds to mouse TCRVβ17, and a cytokine molecule, i.e., IL-2, as provided herein, did further not delay tumor growth or reduce the tumor volume in the mouse MC38 tumor model of colorectal carcinoma, relative to administration of mSTAR1302 (“Vβ13×IL-2”) alone or administration of Vβ17×IL-2 (mSTAR1702) alone. FIG. 9B shows that administration of an exemplary first agent, mSTAR1302 (“Vβ13×IL-2”), that comprises a first binder that binds to a first target molecule, i.e., a binder that binds to mouse TCRVβ13, and a cytokine molecule, i.e., IL-2, as provided herein, alone, administration of an exemplary second agent, Vβ17×IL-2 (mSTAR1702), that comprises a second binder that binds to a second target molecule, i.e., a binder that binds to mouse TCRVβ17, and a cytokine molecule, i.e., IL-2, as provided herein, alone, or concurrent administration of mSTAR1302 (“Vβ13×IL-2”) and m1702 (“Vβ17×IL-2”) had minimal impact on the body weight at the dose of 0.3 mg/kg or 1 mg/kg in the mouse MC38 tumor model of colorectal carcinoma, compared with administration of vehicle control PBS phosphate-buffered saline), administration of mSTAR1302 (“Vβ13×IL-2”) alone, or administration of m1702 (“Vβ17×IL-2”) alone, the agent “m1302” (also referred as “mSTAR1302” or “Vβ13×IL-2”) comprises a binder that binds to mouse TCR Vβ13, and IL-2, and is a mouse surrogate for an agent that comprises a binder that binds to human TCR Vβ6, and IL-2 (referred as “0602”, “STAR0602”, or “Vβ06×IL-2”). The agent “m1702”, also referred as “mSTAR1702” or “Vβ17×IL-2”, comprises a binder that binds to mouse TCR Vβ17, and IL-2, and is a mouse surrogate for an agent that comprises a binder that binds to human TCR Vβ15, and IL-2 (referred as “1502”, “STAR1502”, or “Vβ15×IL-2”). “0.3” indicates administration of the agent at the dose of 0.3 mg/kg, and “1.0” indicates administration of the agent at the dose of 1.0 mg/kg. The agents were administered once weekly (QW).

### DETAILED DESCRIPTION

### Definition

Certain specific details of this description are set forth in order to provide a thorough understanding of various embodiments. However, one skilled in the art will understand that the present disclosure may be practiced without these details. In other instances, well-known structures have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

As used in this specification and the appended claims, the singular forms “a.” “an,” and “the” include plural referents unless the content clearly dictates otherwise. The use of the words “a” or “an” when used in conjunction with the term “comprising” herein may mean “one,” but it is also consistent with the meaning of “one or more,” “at least one,” and “one or more than one.”

It should also be noted that the term “or” is generally employed in its sense including “and/or” unless the content clearly dictates otherwise.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods. As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values.

The term “acquire” or “acquiring” as the terms are used herein, refer to obtaining possession of a physical entity (e.g., a sample, a polypeptide, a nucleic acid, or a sequence), or a value, e.g., a numerical value, by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third party laboratory that directly acquired the physical entity or value). Directly acquiring a physical entity includes performing a process that includes a physical change in a physical substance, e.g., a starting material. Directly acquiring a value includes performing a process that includes a physical change in a sample or another substance, e.g., performing an analytical process which includes a physical change in a substance, e.g., a sample.

“Antibody molecule” as used herein refers to a protein, e.g., an immunoglobulin chain or fragment thereof, comprising at least one immunoglobulin variable domain structure and/or sequence. An antibody molecule encompasses antibodies (e.g., full-length antibodies) and antibody fragments. In some embodiments, an antibody molecule comprises an antigen binding or functional fragment of a full length antibody, or a full length immunoglobulin chain. For example, a full-length antibody is an immunoglobulin (Ig) molecule (e.g., an IgG antibody) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. An antibody fragment, e.g., functional fragment, is a portion of an antibody, e.g., Fab, Fab′, F(ab′)2, F(ab)2, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)2 fragments, and single chain variable fragments (scFvs). In some embodiments, the antibody molecule is an antibody mimetic. In some embodiments, the antibody molecule is, or comprises, an antibody-like framework or scaffold, such as, fibronectins, ankyrin repeats (e.g., designed ankyrin repeat proteins (DARPins)), avimers, affibody affinity ligands, anticalins, or affilin molecules.

The term “human-like antibody molecule” as used herein refers to a humanized antibody molecule, human antibody molecule or an antibody molecule having at least 95% sequence identity with a non-murine germline framework region, e.g., FR1, FR2, FR3 and/or FR4. In some embodiments, the human-like antibody molecule comprises a framework region having at least 95% sequence identity to a human germline framework region, e.g., a FR1, FR2, FR3 and/or FR4 of a human germline framework region. In some embodiments, the human-like antibody molecule is a recombinant antibody. In some embodiments, the human-like antibody molecule is a humanized antibody molecule. In some embodiments, the human-like antibody molecule is human antibody molecule. In some embodiments, the human-like antibody molecule is a phage display or a yeast display antibody molecule. In some embodiments, the human-like antibody molecule is a chimeric antibody molecule. In some embodiments, the human-like antibody molecule is a CDR grafted antibody molecule.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

In embodiments, an antibody molecule is monospecific, e.g., it comprises binding specificity for a single epitope. In some embodiments, an antibody molecule is multispecific, e.g., it comprises a plurality of immunoglobulin variable domain sequences, where a first immunoglobulin variable domain sequence has binding specificity for a first epitope and a second immunoglobulin variable domain sequence has binding specificity for a second epitope. In some embodiments, an antibody molecule is a bispecific antibody molecule. “Bispecific antibody molecule” as used herein refers to an antibody molecule that has specificity for more than one (e.g., two, three, four, or more) epitope and/or antigen.

“Antigen” (Ag) as used herein refers to a molecule that can provoke an immune response, e.g., involving activation of certain immune cells and/or antibody generation. Any macromolecule, including almost all proteins or peptides, can be an antigen. Antigens can also be derived from genomic recombinant or DNA. For example, any DNA comprising a nucleotide sequence or a partial nucleotide sequence that encodes a protein capable of eliciting an immune response encodes an “antigen.” In embodiments, an antigen does not need to be encoded solely by a full length nucleotide sequence of a gene, nor does an antigen need to be encoded by a gene at all. In embodiments, an antigen can be synthesized or can be derived from a biological sample. e.g., a tissue sample, a tumor sample, a cell, or a fluid with other biological components. As used, herein a “tumor antigen” or interchangeably, a “cancer antigen” includes any molecule present on, or associated with, a cancer, e.g., a cancer cell or a tumor microenvironment that can provoke an immune response. As used, herein an “immune cell antigen” includes any molecule present on, or associated with, an immune cell that can provoke an immune response.

The “antigen-binding site,” or “binding portion” of an antibody molecule refers to the part of an antibody molecule, e.g., an immunoglobulin (Ig) molecule, that participates in antigen binding. In embodiments, the antigen binding site is formed by amino acid residues of the variable (V) regions of the heavy (H) and light (L) chains. Three highly divergent stretches within the variable regions of the heavy and light chains, referred to as hypervariable regions, are disposed between more conserved flanking stretches called “framework regions,” (FRs). FRs are amino acid sequences that are naturally found between, and adjacent to, hypervariable regions in immunoglobulins. In embodiments, in an antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917. Each variable chain (e.g., variable heavy chain and variable light chain) is typically made up of three CDRs and four FRs, arranged from amino-terminus to carboxy-terminus in the amino acid order: FR1, CDR1, FR2, CDR2, FR3, CDR3, and FR4.

As used herein, an “immune cell” refers to any of various cells that function in the immune system, e.g., to protect against agents of infection and foreign matter. In embodiments, this term includes leukocytes, e.g., neutrophils, eosinophils, basophils, lymphocytes, and monocytes. Innate leukocytes include phagocytes (e.g., macrophages, neutrophils, and dendritic cells), mast cells, eosinophils, basophils, and natural killer cells. Innate leukocytes identify and eliminate pathogens, either by attacking larger pathogens through contact or by engulfing and then killing microorganisms, and are mediators in the activation of an adaptive immune response. The cells of the adaptive immune system are special types of leukocytes, called lymphocytes. B cells and T cells are important types of lymphocytes and are derived from hematopoietic stem cells in the bone marrow. B cells are involved in the humoral immune response, whereas T cells are involved in cell-mediated immune response. The term “immune cell” includes immune effector cells.

“Immune effector cell,” as that term is used herein, refers to a cell that is involved in an immune response, e.g., in the promotion of an immune effector response. Examples of immune effector cells include, but are not limited to, T cells, e.g., alpha/beta T cells and gamma/delta T cells, B cells, natural killer (NK) cells, natural killer T (NK T) cells, and mast cells.

The term “effector function” or “effector response” refers to a specialized function of a cell. Effector function of a T cell, for example, may be cytolytic activity or helper activity including the secretion of cytokines.

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature. An isolated polynucleotide (ribonucleic acid (RNA), deoxyribonucleic acid (DNA)), or polypeptide is free of the genes/nucleic acids or sequences/amino acids that flank it in its naturally-occurring state.

The compositions and methods of the present invention encompass polypeptides and nucleic acids having the sequences specified, or sequences substantially identical or similar thereto, e.g., sequences at least 80%, 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 80%, 85%, 90%, 91%, 92%, 93%, 94%, 95%, 96%, 97%, 98% 99%, 99.5%, 99.9%, or 100% sequence identity to a reference sequence, e.g., a sequence provided herein. In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 80%, 85%, 90%, 91%, 92%, 93%, 94%, 95%, 96%, 97%, 98% 99%, 99.5%, 99.9%, or 100% sequence identity to a reference sequence, e.g., a sequence provided herein.

The term “variant” refers to a polypeptide that has a substantially identical amino acid sequence to a reference amino acid sequence, or is encoded by a substantially identical nucleotide sequence. In some embodiments, the variant is a functional variant. In some embodiments, a TCRβV variant can bind to TCRα and form a TCR α:β complex.

The term “functional variant” refers to a polypeptide that has a substantially identical amino acid sequence to a reference amino acid sequence, or is encoded by a substantially identical nucleotide sequence, and is capable of having one or more activities of the reference amino acid sequence.

The term “functional fragment” refers to a polypeptide that has a partial amino acid sequence of a reference amino acid sequence, and is capable of having one or more activities of the reference amino acid sequence. In some embodiments, the functional fragment comprises at least about 30%, 35%, 40%, 45%, 50%, 55%, 60%, 65%, 70%, 75%, 80%, 85%, 90%, 91%, 92%, 93%, 94%, 95%, 96%, 97%, 98% 99%, 99.5%, or 99.9% amino acid sequence of a reference amino acid sequence. In some embodiments, the functional fragment comprises an amino acid sequence that has at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid deletion from the reference amino acid sequence. In some embodiments, the functional fragment comprises an amino acid sequence that has at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, or 500 amino acids of the reference amino acid sequence.

Calculations of homology or sequence identity between sequences (the terms are used interchangeably herein) are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4. The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid molecule of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used.

It is understood that the molecules of the present invention may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

As used herein, the term “molecule” as used in, e.g., antibody molecule, cytokine molecule, receptor molecule, includes full-length, naturally-occurring molecules, as well as variants, e.g., functional variants (e.g., truncations, fragments, mutated (e.g., substantially similar sequences) or derivatized form thereof), so long as at least one function and/or activity of the unmodified (e.g., naturally-occurring) molecule remains.

As used herein, the term “mutation” refers to an alteration in the nucleotide sequence of the genome of an organism, virus, or extrachromosomal DNA. In some embodiments, the mutation may be a large-scale mutation, such as amplifications (or gene duplications) or repetitions of a chromosomal segment, deletions of large chromosomal regions, chromosomal rearrangements (e.g., chromosomal translocations, chromosomal inversions, non-homologous chromosomal crossover, and interstitial deletions), and loss of heterozygosity. In some embodiments, the mutation may be a small-scale mutation, such as insertions, deletions, and substitution mutations. As used herein, the term “substitution mutation” refers to the transition that exchange a single nucleotide for another.

“Interleukin-2” also known as IL2, IL-2, IL 2, TCGF, lymphokine, and interleukin 2, as referred to herein, includes any of the recombinant or naturally-occurring forms of IL-2 or variants or homologs thereof that have or maintain IL-2 activity (e.g., at least 40% 50%, 60%, 70%, 80%, 90%, 95%, 96%, 97%, 98%, 99% or 100% activity). In some aspects, the variants or homologs have at least 40%, 45%, 50%, 55%, 60%, 65%, 70%, 75%, 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring IL-2. In some embodiments, IL-2 is substantially identical to the protein identified by the UniProt reference number P60568 or a variant or homolog having substantial identity thereto.

### Anti-TCRβV Antibodies

### Human T Cell Receptor (TCR) Complex

TCR is a disulfide-linked membrane-anchored heterodimeric protein normally consisting of the highly variable alpha (α) and beta (β) chains expressed as part of a complex with the invariant CD3 chain molecules. TCR on αβ T cells is formed by a heterodimer of one alpha chain and one beta chain. Each alpha or beta chain consists of a constant domain and a highly variable domain classified as the Immunoglobulin superfamily (IgSF) fold. The TCRβV chains can be further classified into subfamilies. Despite their high structural and functional homology, the amino acid sequence homology in the TRBV genes is very low. Only 4 amino acids out of approximately 95 are identical while 10 additional amino acids are conserved among all subfamilies. Nevertheless, TCRs formed between alpha and beta chains of highly diverse sequences show a remarkable structural homology and elicit a similar function, e.g., activation of T cells.

T cell receptors (TCR) can be found on the surface of T cells. TCRs recognize antigens, e.g., peptides, presented on, e.g., bound to, major histocompatibility complex (MHC) molecules on the surface of cells, e.g., antigen-presenting cells. TCRs are heterodimeric molecules and can comprise an alpha chain, a beta chain, a gamma chain or a delta chain. TCRs comprising an alpha chain and a beta chain are also referred to as TCRαβ. The TCR beta chain consists of the following regions (also known as segments): variable (V), diversity (D), joining (J) and constant (C) (see Mayer G. and Nyland J. (2010) Chapter 10: Major Histocompatibility Complex and T-cell Receptors-Role in Immune Responses. In: Microbiology and Immunology on-line, University of South Carolina School of Medicine). The TCR alpha chain consists of V, J and C regions. The rearrangement of the T-cell receptor (TCR) through somatic recombination of V (variable), D (diversity), J (joining), and C (constant) regions is a defining event in the development and maturation of a T cell. TCR gene rearrangement takes place in the thymus.

TCRs can comprise a receptor complex, known as the TCR complex, which comprises a TCR heterodimer comprising of an alpha chain and a beta chain, and dimeric signaling molecules, e.g., CD3 co-receptors, e.g., CD3δ/ε, and/or CD3γ/ε.

As used herein, the term “T cell receptor beta variable chain” or “TCRβV,” refers to an extracellular region of the T cell receptor beta chain which comprises the antigen recognition domain of the T cell receptor. The term TCRβV includes isoforms, mammalian, e.g., human TCRβV, species homologs of human and analogs comprising at least one common epitope with TCRβV. Human TCRβV comprises a gene family comprising subfamilies including, but not limited to: a TCRβ V5 subfamily, a TCRβ V6 subfamily, a TCRβ V7 subfamily, a TCRβ V10 subfamily, a TCRβ V2 subfamily, a TCRβ V15 subfamily, a TCRβ V19 subfamily, a TCRβ V20 subfamily, or a TCRβ V28 subfamily, as well as family members of said subfamilies, and variants thereof (e.g., a structural or functional variant thereof). In some embodiments, the TCRβ V6 subfamily comprises: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments, TCRβV comprises TCRβ V6-5*01, or a variant thereof, e.g., a variant having 85%, 90%, 95%, 99% or more identity the naturally-occurring sequence. TCRβ V6-5*01 is also known as TRBV65; TCRBV6S5; TCRBV13S1, or TCRβ V13.1. The amino acid sequence of TCRβ V6-5*01, e.g., human TCRβ V6-5*01, is known in that art, e.g., as provided by IMGT ID L36092. In some embodiments, TCRβ V6-5*01 is encoded by the nucleic acid sequence of SEQ ID NO: 43, or a sequence having 85%, 90%, 95%, 99% or more identity thereof. In some embodiments, TCRβ V6-5*01 comprises the amino acid sequence of SEQ ID NO: 44, or a sequence having 85%, 90%, 95%, 99% or more identity thereof.

SEQ ID NO: 43
ATGAGCATCGGCCTCCTGTGCTGTGCAGCCTTGTCTCTCCTGTGGGCAG
GTCCAGTGAATGCTGGTGTCACTCAGACCCCAAAATTCCAGGTCCTGAA
GACAGGACAGAGCATGACACTGCAGTGTGCCCAGGATATGAACCATGAA
TACATGTCCTGGTATCGACAAGACCCAGGCATGGGGCTGAGGCTGATTC
ATTACTCAGTTGGTGCTGGTATCACTGACCAAGGAGAAGTCCCCAATGG
CTACAATGTCTCCAGATCAACCACAGAGGATTTCCCGCTCAGGCTGCTG
TCGGCTGCTCCCTCCCAGACATCTGTGTACTTCTGTGCCAGCAGTTACTC
SEQ ID NO: 44
MSIGLLCCAALSLLWAGPVNAGVTQTPKFQVLKTGQSMTLQCAQDMNHE
YMSWYRQDPGMGLRLIHY-SVGAGITDQGEVPNGYNVSRSTTEDFPLRL
LSAAPSQTSVYFCASSY

### TCR Beta V (TCRβV)

Diversity in the immune system enables protection against a huge array of pathogens. Since the germline genome is limited in size, diversity is achieved not only by the process of V(D)J recombination but also by junctional (junctions between V-D and D-J segments) deletion of nucleotides and addition of pseudo-random, non-templated nucleotides. The TCR beta gene undergoes gene arrangement to generate diversity.

The TCR V beta repertoire varies between individuals and populations because of, e.g., 7 frequently occurring inactivating polymorphisms in functional gene segments and a large insertion/deletion-related polymorphism encompassing 2 V beta gene segments.

Provided herein are, inter alia, antibody molecules and fragments thereof, that bind, e.g., specifically bind, to a human TCR beta V chain (TCRβV), e.g., a TCRβV gene family (also referred to as a group), e.g., a TCRβV subfamily (also referred to as a subgroup), e.g., as described herein. TCR beta V families and subfamilies are known in the art, e.g., as described in Yassai et al., (2009) Immunogenetics 61(7) pp: 493-502; Wei S. and Concannon P. (1994) Human Immunology 41(3) pp: 201-206. The antibodies described herein can be recombinant antibodies, e.g., recombinant non-murine antibodies, e.g., recombinant human or humanized antibodies.

The terms TCRBV, TCRVB, TRBV, TCRβV, TCRVβ or TRβV are used interchangeably herein and refer to a TCR beta V chain, e.g., as described herein.

In some embodiments, provided herein is an anti-TCRβV antibody molecule that binds to human TCRβV, e.g., a TCRβV family, e.g., gene family or a variant thereof. In some embodiments a TCRBV gene family comprises one or more subfamilies, e.g., as described herein, e.g., Table 8A. In some embodiments, the TCRβV gene family comprises: a TCRβ V6 subfamily, a TCRβ V10 subfamily, a TCRβ V5 subfamily, a TCRβ V7 subfamily, a TCRβ V19 subfamily, a TCRβ V20 subfamily, or a TCRβ V28 subfamily.

In some embodiments, TCRβ V6 subfamily is also known as TCRβ V13.1. In some embodiments, the TCRβ V6 subfamily comprises: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01, or a variant thereof. In some embodiments, TCRβ V6 comprises TCRβ V6-4*01, or a variant thereof. In some embodiments, TCRβ V6 comprises TCRβ V6-4*02, or a variant thereof. In some embodiments, TCRβ V6 comprises TCRβ V6-9*01, or a variant thereof. In some embodiments, TCRβ V6 comprises TCRβ V6-8*01, or a variant thereof. In some embodiments, TCRβ V6 comprises TCRβ V6-5*01, or a variant thereof. In some embodiments, TCRβ V6 comprises TCRβ V6-6*02, or a variant thereof. In some embodiments. TCRβ V6 comprises TCRβ V6-6*01, or a variant thereof. In some embodiments, TCRβ V6 comprises TCRβ V6-2*01, or a variant thereof. In some embodiments, TCRβ V6 comprises TCRβ V6-3*01, or a variant thereof. In some embodiments, TCRβ V6 comprises TCRβ V6-1*01, or a variant thereof.

In some embodiments, TCRβ V6 comprises TCRβ V6-5*01, or a variant thereof. In some embodiments, TCRβ V6, e.g., TCRβ V6-5*01, is recognized, e.g., bound, by SEQ ID NO: 1 and/or SEQ ID NO: 2. In some embodiments, TCRβ V6, e.g., TCRβ V6-5*01, is recognized, e.g., bound, by SEQ ID NO: 9 and/or SEQ ID NO: 10. In some embodiments, TCRβ V6 is recognized, e.g., bound, by SEQ ID NO: 9 and/or SEQ ID NO: 11.

In some embodiments, TCRβ V10 subfamily is also known as TCRβ V12. In some embodiments, the TCRβ V10 subfamily comprises: TCRβ V10-1*01, TCRβ V10-1*02, TCRβ V10-3*01 or TCRβ V10-2*01, or a variant thereof.

In some embodiments, TCRβ V12 subfamily is also known as TCRβ V8.1. In some embodiments, the TCRβ V12 subfamily comprises: TCRβ V12-4*01, TCRβ V12-3*01, or TCRβ V12-5*01, or a variant thereof. In some embodiments, TCRβ V12 is recognized, e.g., bound, by SEQ ID NO: 15 and/or SEQ ID NO: 16. In some embodiments, TCRβ V12 is recognized, e.g., bound, by any one of SEQ ID NOs 23-25, and/or any one of SEQ ID NO: 26-30:
In some embodiments, the TCRβ V5 subfamily is chosen from: TCRβ V5-5*01, TCRβ V5-6*01, TCRβ V5-4*01, TCRβ V5-8*01, TCRβ V5-1*01, or a variant thereof.

In some embodiments, the TCRβ V7 subfamily comprises TCRβ V7-7*01, TCRβ V7-6*01, TCRβ V7-8*02, TCRβ V7-4*01, TCRβ V7-2*02, TCRβ V7-2*03, TCRβ V7-2*01, TCRβ V7-3*01, TCRβ V7-9*03, or TCRβ V7-9*01, or a variant thereof.

In some embodiments, the TCRβ V15 subfamily comprises TCRβ V15*01, or a variant thereof in some embodiments, the TCRβ V19 subfamily comprises TCRβ V19*01, or TCRβ V19*02, or a variant thereof. In some embodiments, the TCRβ V28 subfamily comprises TCRβ V28*01, or a variant thereof. In some embodiments, the TCRβ V20 subfamily comprises TCRβ V20-1*01, or TCRβ V20-1*02, or a variant thereof. In some embodiments, the TCRβ V28 subfamily comprises TCRβ V28-1*01, or a variant thereof.

Exemplary amino acid sequences for TCRβV subfamily members can be found on the ImMunoGeneTics Information System website: http://www.imgt.org/, or in a similar resource.

### Anti-TCRβV Antibodies

Current bispecific constructs designed to redirect T cells to promote tumor cell lysis for cancer immunotherapy typically utilize antibody fragments (Fab, scFv, VH, single domain antibody, etc.) that are derived from monoclonal antibodies (mAb) directed against the CD3e subunit of the T cell receptor (TCR). However, there are limitations to this approach which may prevent the full realization of the therapeutic potential for such bispecific constructs. Previous studies have shown that even low “activating” doses of anti-CD3e mAb can cause long-term T cell dysfunction and exert immunosuppressive effects. In addition, anti-CD3e mAbs have been associated with side effects that result from massive T cell activation. The large number of activated T cells secrete substantial amounts of cytokines, the most important of which is Interferon gamma (IFNγ). This excess amount of IFNγ in turn activates macrophages which then overproduce proinflammatory cytokines such as IL-1beta, IL-6, IL-10 and TNF-alpha, causing a “cytokine storm” known as the cytokine release syndrome (CRS) (Shimabukuro-Vornhagen et al., J Immunother Cancer. 2018 Jun. 15; 6(1):56, herein incorporated by reference in its entirety). Thus, the need exists for developing antibodies that are capable of binding and activating only a subset of effector T cells, e.g., to re-duce the CRS and/or neurotoxicity (NT).

Described herein are molecules targeting the TCRβV chain of TCR and methods thereof. Without wishing to be bound by theory, such molecules are capable of binding, activating, and/or expanding only a subset of T cells, avoiding or reducing CRS and/or NT and minimizing potential immunosuppressive effects of anti-CD3 mAbs.

Described herein is a class of antibodies, i.e., anti-TCRβV antibody molecules as described herein, which despite having low sequence similarity (e.g., low sequence identity among the different antibody molecules that recognize different TCRβV subfamilies), recognize a structurally conserved, yet sequence-wise variable, region, e.g., domain, on the TCRβV protein and have a similar function (e.g., activation of T cells and a similar cytokine profile as described herein). Thus, the anti-TCRβV antibody molecules as described herein share a structure-function relationship.

Without wishing to be bound by theory, in some embodiments, the anti-TCRβV antibody molecules as described herein bind to an outward facing epitope of a TCRβV protein when it is in a complex with a TCRalpha protein. In some embodiments, the anti-TCRβV antibody molecules as described herein recognize (e.g., bind to), a domain (e.g., an epitope) on the TCRβV protein that is: (1) structurally conserved among different TCRβV subfamilies; and (2) has minimal sequence identity among the different TCRβV subfamilies. TCRβV proteins from the different TCRβV subfamilies share minimal sequence similarity. However. TCRβV proteins which have minimal sequence similarity, share a similar 3D conformation and structure.

The alignment of TCRβV amino acid sequences in ta9 underscores the diversity of TCR sequences. In particular, the TRBV sequences from different subfamilies are considerably different from each other.

In some embodiments, the anti-TCRβV antibody molecules as described herein do not recognize, e.g., bind to, an interface of a TCRβV:TCRalpha complex. In some embodiments, the anti-TCRβV antibody molecules as described herein do not recognize, e.g., bind to, a constant region of a TCRβV protein. An exemplary antibody that binds to a constant region of a TCRβV region is JOVI.1 as de-scribed in Viney et al., (Hybridoma. 1992 December; 11(6):701-13). In some embodiments, the anti-TCRβV antibody molecules as described herein do not recognize, e.g., bind to, one or more (e.g., all) of a complementarity determining region (e.g., CDR1, CDR2 and/or CDR3) of a TCRβV protein.

Provided herein are, inter alia, antibody molecules directed to the variable chain of the beta subunit of TCR (TCRβV) which bind and, e.g., activate a subset of T cells. The anti-TCRβV antibody molecules as described herein result in lesser or no production of cytokines associated with CRS, e.g., IL-6, IL-1beta, IL-10 and TNF alpha; and enhanced and/or delayed production of IL-2 and IFNγ. In some embodiments, the anti-TCRβV antibodies as described herein have a cytokine profile, e.g., as described herein, which differs from a cytokine profile of a T cell engager that binds to a receptor or molecule other than a TCRβV region (“a non-TCRβV-binding T cell engager”). In some embodiments, the non-TCRβV-binding T cell engager comprises an antibody that binds to a CD3 molecule (e.g., CD3 epsilon (CD3e) molecule); or a TCR alpha (TCRα) molecule. In some embodiments, the non-TCRβV-binding T cell engager is an OKT3 antibody or an SP34-2 antibody.

In some embodiments, the anti-TCRβV antibodies as described herein result in expansion of TCRβV+ T cells, e.g., a subset of memory effector T cells known as TEMRA. Without wishing to be bound by theory, it is believed that in some embodiments, TEMRA cells can promote tumor cell lysis but not CRS. Accordingly, provided herein are methods of making said anti-TCRβV antibody molecules and uses thereof. Also described herein are multispecific molecules, e.g., bispecific molecules comprising said anti-TCRβV antibody molecules. In some embodiments, compositions comprising anti-TCRβV antibody molecules of the present disclosure, can be used, e.g., to: (1) activate and redirect T cells to promote tumor cell lysis for cancer immuno-therapy; and/or (2) expand TCRβV+ T cells. In some embodiments, compositions comprising anti-TCRβV antibody molecules as described herein limit the harmful side-effects of CRS and/or NT, e.g., CRS and/or NT associated with anti-CD3e targeting.

In some embodiments, the anti-TCRβV antibody molecule binds to one or more of TRBV5-1, TRBV5-4, TRBV5-5, TRBV5-6, TRBV5-8, TRBV6-1, TRBV6-2, TRBV6-3, TRBV6-4, TRBV6-5, TRBV6-6, TRBV6-8, TRBV6-9, TRBV7-2, TRBV7-3, TRBV7-4, TRBV7-6, TRBV7-7, TRBV10-1, TRBV10-2, TRBV10-3, TRBV12-3, TRBV12-4, TRBV12-5, TRBV15, TRBV19, TRBV20-1, and TRBV28. In some embodiments, the anti-TCRβV antibody molecule binds to one or more of TRBV6-1, TRBV6-2, TRBV6-3, TRBV6-4, TRBV6-5, TRBV6-6, TRBV6-8 and TRBV6-9. In some embodiments, the anti-TCRβV antibody molecule is an anti-TRBV5-1, anti-TRBV5-4, anti-TRBV5-5, anti-TRBV5-6, anti-TRBV5-8, anti-TRBV6-1, anti-TRBV6-2, anti-TRBV6-3, anti-TRBV6-4, anti-TRBV6-5, anti-TRBV6-6, anti-TRBV6-8, anti-TRBV6-9, anti-TRBV7-2, anti-TRBV7-3, anti-TRBV7-4, anti-TRBV7-6, anti-TRBV7-7, anti-TRBV7-8, anti-TRBV7-9, anti-TRBV10-1, anti-TRBV10-2, anti-TRBV10-3, anti-TRBV12-3, anti-TRBV12-4, anti-TRBV12-5, anti-TRBV15, anti-TRBV19, anti-TRBV20- or anti-TRBV28. Exemplary anti-TCRβV antibody molecules and the corresponding TCRβV subfamilies recognized by said anti-TCRβV antibody molecules are disclosed in Table 10A.

In some embodiments, the anti-TCRβV antibody molecule binds specifically to TRBV5-1, TRBV5-4, TRBV5-5, TRBV5-6, TRBV5-8, TRBV6-1, TRBV6-2, TRBV6-3, TRBV6-4, TRBV6-5, TRBV6-6, TRBV6-8, TRBV6-9, TRBV7-2, TRBV7-3, TRBV7-4, TRBV7-6, TRBV7-7, TRBV7-8, TRBV10-1, TRBV10-2, TRBV10-3, TRBV12-3, TRBV12-4, TRBV12-5, TRBV15, TRBV19, TRBV20-1, TRBV27 or TRBV28. In some embodiments, the anti-TCRβV antibody molecule binds specifically to TRBV6-1. In some embodiments, the anti-TCRβV antibody molecule binds specifically to TRBV6-2. In some embodiments, the anti-TCRβV antibody molecule binds specifically to TRBV6-3. In some embodiments, the anti-TCRβV antibody molecule binds specifically to TRBV6-4. In some embodiments, the anti-TCRβV antibody molecule binds specifically to TRBV6-5. In some embodiments, the anti-TCRβV antibody molecule binds specifically to TRBV6-6. In some embodiments, the anti-TCRβV antibody molecule binds specifically to TRBV6-8. In some embodiments, the anti-TCRβV antibody molecule binds specifically to TRBV6-9.

In some embodiments, the anti-TCRβV antibody molecule does not bind to TCRβ V12, or binds to TCRβ V2 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of the 16G8 murine antibody or a humanized version thereof as de-scribed in U.S. Pat. No. 5,861,155.

In some embodiments, the anti-TCRβV antibody molecule binds to TCRβ V12 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the 16G8 murine antibody or a humanized version thereof as described in U.S. Pat. No. 5,861,155.

In some embodiments, the anti-TCRβV antibody molecule binds to a TCRβV region other than TCRβ V12 (e.g., TCRβV region as described herein, e.g., TCRβ V6 subfamily (e.g., TCRβ V6-5*01) with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the 16G8 murine antibody or a humanized version thereof as de-scribed in U.S. Pat. No. 5,861,155.

In some embodiments, the anti-TCRβV antibody molecule does not comprise the CDRs of the Antibody B murine antibody.

In some embodiments, the anti-TCRβV antibody molecule does not bind to TCRβ V5-5*01 or TCRβ V5-1*01, or binds to TCRβ V5-5*01 or TCRβ V5-1*01 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of the TM23 murine antibody or a humanized version thereof as described in U.S. Pat. No. 5,861,155.

In some embodiments, the anti-TCRβV antibody molecule binds to TCRβ V5-5*01 or TCRβ V5-1*01 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the TM23 murine antibody or a humanized version thereof as de-scribed in U.S. Pat. No. 5,861,155.

In some embodiments, the anti-TCRβV antibody molecule binds to a TCRβV region other than TCRβ V5-5*01 or TCRβ V5-1*01 (e.g., TCRβV region as described herein. e.g., TCRβ V6 subfamily (e.g., TCRβ V6-5*01) with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the TM23 murine antibody or a humanized version thereof as described in U.S. Pat. No. 5,861,155.

In some embodiments, the anti-TCRβV antibody molecule binds to one or more of murine TRBV13-1, TRBV13-2, TRBV13-3, TRBV17, TRBV29, and TRBV15. In some embodiments, the anti-TCRβV antibody molecule is anti-TRBV13-1, anti-TRBV13-2, anti-TRBV13-3, TRBV17, TRBV19, TRBV29, and TRBV15.

In some embodiments, the anti-TCRβV antibody molecule binds specifically to murine TRBV13-1, TRBV13-2, TRBV13-3, TRBV17, TRBV19, TRBV29 or TRBV15. In some embodiments, the anti-TCRβV antibody molecule binds specifically to murine TRBV13-1. In some embodiments, the anti-TCRβV antibody molecule binds specifically to murine TRBV13-2. In some embodiments, the anti-TCRβV antibody molecule binds specifically to murine TRBV13-3. In some embodiments, the anti-TCRβV antibody molecule binds specifically to murine TRBV17. In some embodiments, the anti-TCRβV antibody molecule binds specifically to murine TRBV19. In some embodiments, the anti-TCRβV antibody molecule binds specifically to murine TRBV29. In some embodiments, the anti-TCRβV antibody molecule binds specifically to murine TRBV15.

In some embodiments, the light or the heavy chain variable framework (e.g., the region encompassing at least FR1, FR2, FR3, and optionally FR4) of the anti-TCRβV antibody molecule, e.g., anti-TCRβ V6 (e.g., anti-TCRβ V6-5*01) antibody molecule can be chosen from: (a) alight or heavy chain variable framework including at least 80%, 85%, 87% 90%, 92%, 93%, 95%, 97%, 98%, or 100% of the amino acid residues from a human light or heavy chain variable framework, e.g., a light or heavy chain variable framework residue from a human mature antibody, a human germline sequence, or a human consensus sequence; (b) a light or heavy chain variable framework including from 20% to 80%, 40% to 60%, 60% to 90%, or 70% to 95% of the amino acid residues from a human light or heavy chain variable framework, e.g., a light or heavy chain variable framework residue from a human mature antibody, a human germline sequence, or a human consensus sequence; (c) a non-human framework (e.g., a rodent framework); or (d) a non-human framework that has been modified. e.g., to remove antigenic or cytotoxic determinants, e.g., deimmunized, or partially humanized. In some embodiments, the light or heavy chain variable framework region (particularly FR1, FR2 and/or FR3) includes a light or heavy chain variable framework sequence at least 70, 75, 80, 85, 87, 88, 90, 92, 94, 95, 96, 97, 98, 99% identical or identical to the frameworks of a VL or VH segment of a human germline gene.

In some embodiments, the anti-TCRβV antibody molecule, e.g., anti-TCRβ V6 (e.g., anti-TCRβ V6-5*01) antibody molecule, comprises a heavy chain variable domain having at least one, two, three, four, five, six, seven, ten, fifteen, twenty or more changes, e.g., amino acid substitutions or deletions, from an amino acid sequence of any one of A-H.1 to A-H.85, e.g., A-H.1, A-H.2 or A-H.68, e.g., the amino acid sequence of the FR region in the entire variable region, e.g., in SEQ ID NO: 9.

Alternatively, or in combination with the heavy chain substitutions described herein, the anti-TCRβV antibody molecule, e.g., anti-TCRβ V6 (e.g., anti-TCRβ V6-5*01) antibody molecule, comprises a light chain variable domain having at least one, two, three, four, five, six, seven, ten, fifteen, twenty or more amino acid changes, e.g., amino acid substitutions or deletions, from an amino acid sequence of any one of A-H.1 to A-H.85, e.g., A-H.1, A-H.2 or A-H.68, e.g., the amino acid sequence of the FR region in the entire variable region, e.g SEQ ID NO: 10 or SEQ ID NO: 11.

In some embodiments, the anti-TCRβV antibody molecule, e.g., anti-TCRβ V6 (e.g., anti-TCRβ V6-5*01) antibody molecule, comprises the light chain framework region 1 of A-H.1 or A-H.2.

In some embodiments, the anti-TCRβV antibody molecule, e.g., anti-TCRβ V6 (e.g., anti-TCRβ V6-5*01) antibody molecule, comprises the light chain framework region 2 of A-H.1 or A-H.2.

In some embodiments, the anti-TCRβV antibody molecule, e.g., anti-TCRβ V6 (e.g., anti-TCRβ V6-5*01) antibody molecule, comprises the light chain framework region 3 of A-H.1 or A-H.2.

In some embodiments, the anti-TCRβV antibody molecule, e.g., anti-TCRβ V6 (e.g., anti-TCRβ V6-5*01) antibody molecule, comprises the light chain framework region 4 of A-H.1 or A-H.2.

In some embodiments, the anti-TCRβV antibody molecule, e.g., anti-TCRβ V6 (e.g., anti-TCRβ V6-5*01) antibody molecule, comprises a light chain variable domain comprising a framework region, e.g., framework region 1 (FR1), comprising a change, e.g., a substitution (e.g., a conservative substitution) at position 10 according to Kabat numbering. In some embodiments, the FR1 comprises a Phenylalanine at position 10, e.g., a Serine to Phenyalanine substitution. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising a framework region, e.g., framework region 1 (FR1), comprising a change, e.g., a substitution (e.g., a conservative substitution) at position 10 according to Kabat numbering. In some embodiments, the FR1 comprises a Phenylalanine at position 10, e.g., a Serine to Phenyalanine substitution. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising a framework region, e.g., framework region 2 (FR2), comprising a change, e.g., a substitution (e.g., a conservative substitution) at a position as described herein according to Kabat numbering. In some embodiments, FR2 comprises a Histidine at position 36, e.g., a substitution at position 36 according to Kabat numbering, e.g., a Tyrosine to Histidine substitution. In some embodiments, FR2 comprises an Alanine at position 46, e.g., a substitution at position 46 according to Kabat numbering, e.g., an Arginine to Alanine substitution. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising a framework region, e.g., framework region 3 (FR3), comprising a change, e.g., a substitution (e.g., a conservative substitution) at a position as described herein according to Kabat numbering. In some embodiments, FR3 comprises a Phenyalanine at position 87, e.g., a substitution at position 87 according to Kabat numbering, e.g., a Tyrosine to Phenyalanine substitution. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising: (a) a framework region 1 (FR1) comprising a Phenylalanine at position 10, e.g., a substitution at position 10 according to Kabat numbering. e.g., a Serine to Phenyalanine substitution; (b) a framework region 2 (FR2) comprising a Histidine at position 36, e.g., a substitution at position 36 according to Kabat numbering, e.g., a Tyrosine to Histidine substitution, and a Alanine at position 46, e.g., a substitution at position 46 according to Kabat numbering, e.g., a Arginine to Alanine substitution; and (c) a framework region 3 (FR3) comprising a Phenylalanine at position 87, e.g., a substitution at position 87 according to Kabat numbering, e.g., a Tyrosine to Phenyalanine substitution, e.g., as shown in the amino acid sequence of SEQ ID NO: 10. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising: (a) a framework region 2 (FR2) comprising a Histidine at position 36, e.g., a substitution at position 36 according to Kabat numbering, e.g., a Tyrosine to Histidine substitution, and a Alanine at position 46, e.g., a substitution at position 46 according to Kabat numbering, e.g., a Arginine to Alanine substitution; and (b) a framework region 3 (FR3) comprising a Phenylalanine at position 87, e.g., a substitution at position 87 according to Kabat numbering, e.g., a Tyrosine to Phenyalanine substitution, e.g., as shown in the amino acid sequence of SEQ ID NO: 11. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising: (a) a framework region 1 (FR1) comprising a change, e.g., a substitution (e.g., a conservative substitution) at one or more (e.g., all) positions as described herein according to Kabat numbering; (b) a framework region 2 (FR2) comprising a change, e.g., a substitution (e.g., a conservative substitution) at one or more (e.g., all) position as described herein according to Kabat numbering and (c) a framework region 3 (FR3) comprising a change, e.g., a substitution (e.g., a conservative substitution) at one or more (e.g., all) position as described herein according to Kabat numbering. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises the heavy chain framework region 1 of A-H.1 or A-H.2. In some embodiments, the anti-TCRβV antibody molecule, e.g., anti-TCRβ V6 (e.g., anti-TCRβ V6-5*01) antibody molecule, comprises the heavy chain framework region 2 of A-H.1 or A-H.2. In some embodiments, the anti-TCRβV antibody molecule, e.g., anti-TCRβ V6 (e.g., anti-TCRβ V6-5*01) antibody molecule, comprises the heavy chain framework region 3 of A-H.1 or A-H.2. In some embodiments, the anti-TCRβV antibody molecule, e.g., anti-TCRβ V6 (e.g., anti-TCRβ V6-5*01) antibody molecule, comprises the heavy chain framework region 4 of A-H.1 or A-H.2.

In some embodiments, the anti-TCRβV antibody molecule, e.g., anti-TCRβ V6 (e.g., anti-TCRβ V6-5*01) antibody molecule, comprises a heavy chain variable domain comprising a framework region, e.g., framework region 3 (FR3), comprising a change, e.g., a substitution (e.g., a conservative substitution) at a position as described herein according to Kabat numbering. In some embodiments, FR3 comprises a Threonine at position 73, e.g., a substitution at position 73 according to Kabat numbering, e.g., a Glutamic Acid to Threonine substitution. In some embodiments, FR3 comprises a Glycine at position 94, e.g., a substitution at position 94 according to Kabat numbering, e.g., an Arginine to Glycine substitution. In some embodiments, the substitution is relative to a human germline heavy chain framework region sequence.

In some embodiments, the anti-TCRβV antibody molecule, e.g., anti-TCRβ V6 (e.g., anti-TCRβ V6-5*01) antibody molecule, comprises a heavy chain variable domain comprising a framework region 3 (FR3) comprising a Threonine at position 73, e.g., a substitution at position 73 according to Kabat numbering, e.g., a Glutamic Acid to Threonine substitution, and a Glycine at position 94, e.g., a substitution at position 94 according to Kabat numbering, e.g., a Arginine to Glycine substitution, e.g., as shown in the amino acid sequence of SEQ ID NO: 10.

In some embodiments, the anti-TCRβV antibody molecule, e.g., anti-TCRβ V6 (e.g., anti-TCRβ V6-5*01) antibody molecule, comprises the heavy chain framework regions 1-4 of A-H.1 or A-H.2, e.g., SEQ ID NO: 9. In some embodiments, the anti-TCRβV antibody molecule, e.g., anti-TCRβ V6 (e.g., anti-TCRβ V6-5*01) antibody molecule, comprises the light chain framework regions 1-4 of A-H.1, e.g., SEQ ID NO: 10. In some embodiments, the anti-TCRβV antibody molecule, e.g., anti-TCRβ V6 (e.g., anti-TCRβ V6-5*01) antibody molecule, comprises the light chain framework regions 1-4 of A-H.2, e.g., SEQ ID NO: 11. In some embodiments, the anti-TCRβV antibody molecule, e.g., anti-TCRβ V6 (e.g., anti-TCRβ V6-5*01) antibody molecule, comprises the heavy chain framework regions 1-4 of A-H.1, e.g., SEQ ID NO: 9; and the light chain framework regions 1-4 of A-H.1, e.g., SEQ ID NO: 10. In some embodiments, the anti-TCRβV antibody molecule, e.g., anti-TCRβ V6 (e.g., anti-TCRβ V6-5*01) antibody molecule, comprises the heavy chain framework regions 1-4 of A-H.2, e.g., SEQ ID NO: 9; and the light chain framework regions 1-4 of A-H.2, e.g., SEQ ID NO: 11.

In some embodiments, the heavy or light chain variable domain, or both, of the anti-TCRβV antibody molecule, e.g., anti-TCRβ V6 (e.g., anti-TCRβ V6-5*01) antibody molecule, includes an amino acid sequence, which is substantially identical to an amino acid as described herein, e.g., at least 80%, 85%, 90%, 92%, 95%, 97%, 98%, 99% or higher identical to a variable region of an antibody described herein, e.g., an antibody chosen from any one of A-H.1 to A-H.85, e.g., A-H.1, A-H.2 or A-H.68, or as described in Table 1, or encoded by the nucleotide sequence in Table 1; or which differs at least 1 or 5 residues, but less than 40, 30, 20, or 10 residues, from a variable region of an antibody described herein.

In some embodiments, the anti-TCRβV antibody molecule, e.g., anti-TCRβ V6 (e.g., anti-TCRβ V6-5*01) antibody molecule, comprises at least one, two, three, or four antigen-binding regions, e.g., variable regions, having an amino acid sequence as set forth in Table 1, or a sequence substantially identical thereto (e.g., a sequence at least about 85%, 90%, 95%, 99% or more identical thereto, or which differs by no more than 1, 2, 5, 10, or 15 amino acid residues from the sequences shown in Table 1. In another embodiment, the anti-TCRβV antibody molecule, e.g., anti-TCRβ V6 (e.g., anti-TCRβ V6-5*01) antibody molecule includes a VH and/or VL domain encoded by a nucleic acid having a nucleotide sequence as set forth in Table 1, or a sequence substantially identical thereto (e.g., a sequence at least about 85%, 90%, 95%, 99% or more identical thereto, or which differs by no more than 3, 6, 15, 30, or 45 nucleotides from the sequences shown in Table 1.

In some embodiments, the anti-TCRβV antibody molecule, e.g., anti-TCRβ V6 (e.g., anti-TCRβ V6-5*01) antibody molecule, comprises: a VH domain comprising the amino acid sequence of SEQ ID NO: 9, an amino acid sequence at least about 85%, 90%, 95%, 99% or more identical to the amino acid sequence of SEQ ID NO: 9, or an amino acid sequence which differs by no more than 1, 2, 5, 10, or 15 amino acid residues from the amino acid sequence of SEQ ID NO: 9; and/or a VL domain comprising the amino acid sequence of SEQ ID NO: 10, an amino acid sequence at least about 85%, 90%, 95%, 99% or more identical to the amino acid sequence of SEQ ID NO: 10, or an amino acid sequence which differs by no more than 1, 2, 5, 10, or 15 amino acid residues from the amino acid sequence of SEQ ID NO: 10.

In some embodiments, the anti-TCRβV antibody molecule, e.g., anti-TCRβ V6 (e.g., anti-TCRβ V6-5*01) antibody molecule, comprises: a VH domain comprising the amino acid sequence of SEQ ID NO: 9, an amino acid sequence at least about 85%, 90%, 95%, 99% or more identical to the amino acid sequence of SEQ ID NO: 9, or an amino acid sequence which differs by no more than 1, 2, 5, 10, or 15 amino acid residues from the amino acid sequence of SEQ ID NO: 9; and/or a VL domain comprising the amino acid sequence of SEQ ID NO: 11, an amino acid sequence at least about 85%, 90%, 95%, 99% or more identical to the amino acid sequence of SEQ ID NO: 11, or an amino acid sequence which differs by no more than 1, 2, 5, 10, or 15 amino acid residues from the amino acid sequence of SEQ ID NO: 11.

In some embodiments, the anti-TCRβV antibody molecule, e.g., anti-TCRβ V6 (e.g., anti-TCRβ V6-5*01) antibody molecule is a full antibody or fragment thereof (e.g., a Fab, F(ab′)2, Fv, single domain antibody, or a single chain Fv fragment (scFv)). In embodiments, the anti-TCRβV antibody molecule, e.g., anti-TCRβ V6 (e.g., anti-TCRβ V6-5*01) antibody molecule is a monoclonal antibody or an antibody with single specificity. In some embodiments, the anti-TCRβV antibody molecule, e.g., anti-TCRβ V6 (e.g., anti-TCRβ V6-5*01) antibody molecule, can also be a humanized, chimeric, camelid, shark, or an in vitro-generated antibody molecule. In some embodiments, the anti-TCRβV antibody molecule, e.g., anti-TCRβ V6 (e.g., anti-TCRβ V6-5*01) antibody molecule, is a humanized antibody molecule. The heavy and light chains of the anti-TCRβV antibody molecule, e.g., anti-TCRβ V6 (e.g., anti-TCRβ V6-5*01) antibody molecule, can be full-length (e.g., an antibody can include at least one, and preferably two, complete heavy chains, and at least one, and preferably two, complete light chains) or can include an antigen-binding fragment (e.g., a Fab, F(ab′)2, Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRβV antibody molecule, e.g., anti-TCRβ V6 (e.g., anti-TCRβ V6-5*01) antibody molecule, is in the form of a multispecific molecule, e.g., a bispecific molecule, e.g., as described herein.

In some embodiments, the anti-TCRβV antibody molecule, e.g., anti-TCRβ V6 (e.g., anti-TCRβ V6-5*01) antibody molecule, has a heavy chain constant region (Fc) chosen from, e.g., the heavy chain constant regions of IgG1, IgG2, IgG3, IgG4, IgM, IgA1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgG1 or IgG2 (e.g., human IgG1, or IgG2). In some embodiments, the heavy chain constant region is human IgG1. In some embodiments, the Fc region comprises a Fc region variant, e.g., as described herein.

In some embodiments, the anti-TCRβV antibody molecule, e.g., anti-TCRβ V6 (e.g., anti-TCRβ V6-5*01) antibody molecule, has a light chain constant region chosen from, e.g., the light chain constant regions of kappa or lambda, preferably kappa (e.g., human kappa). In some embodiments, the constant region is altered, e.g., mutated, to modify the properties of the anti-TCRβV antibody molecule, e.g., anti-TCRβ V6 (e.g., anti-TCRβ V6-5*01) antibody molecule (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 212 or 214; or positions 135 (M to Y), 137 (S to T), 139 (T to E), 316 (H to K) and 317 (N to F) of SEQ ID NOs: 215, 216, 217 or 218), e.g., relative to human IgG1.

Antibody A-H.1 comprises a heavy chain comprising the amino acid sequence of SEQ ID NO: 3278 and a light chain comprising the amino acid sequence of SEQ ID NO: 72. Antibody A-H.2 comprises a heavy chain comprising the amino acid sequence of SEQ ID NO: 3278 and a light chain comprising the amino acid sequence of SEQ ID NO: 3279. Antibody A-H.68 comprises the amino acid sequence of SEQ ID NO: 1337, or a sequence having at least 85%, 90%, 95%, 96%, 97%, 98%, 99%, 99.5%, 99.9%, or 100% sequence identity thereto. Antibody A-H.69 comprises the amino acid sequence of SEQ ID NO: 1500, or a sequence having at least 85%, 90%, 95%, 96%, 97%, 98%, 99%, 99.5%, 99.9%, or 100% sequence identity thereto.

Additional exemplary humanized anti-TCRB V6 antibodies are provided in Table 1. In some embodiments, the anti-TCRβ V6 is antibody A. e.g., humanized antibody A (antibody A-H), as provided in Table 1. In some embodiments, the anti-TCRβV antibody comprises one or more (e.g., all three) of a LC CDR1, LC CDR2, and LC CDR3 provided in Table 1; and/or one or more (e.g., all three) of a HC CDR1, HC CDR2, and HC CDR3 provided in Table 1, or a sequence with at least 85%, 900, 95%, 96%, 97%, 98%, 99%, 99.5%, 99.9%, or 100% sequence identity thereto. In some embodiments, antibody A comprises a variable heavy chain (VH) and/or a variable light chain (VL) provided in Table 1, or a sequence with at least 85%, 90%, 95%, 96%, 97%, 98%, 99%, 99.5%, 99.9%, or 100% sequence identity thereto.

In some embodiments, the anti-TCRβV antibody molecule, e.g., anti-TCRβ V6 (e.g., anti-TCRβ V6-5*01) antibody molecule comprises a VH of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto.

In some embodiments, the anti-TCRβV antibody molecule, e.g., anti-TCRβ V6 (e.g., anti-TCRβ V6-5*01) antibody molecule comprises a VL of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto.

In some embodiments, the anti-TCRβV antibody molecule, e.g., anti-TCRβ V6 (e.g., anti-TCRβ V6-5*01) antibody molecule comprises a VH of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto; and a VL of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto.

Exemplary anti-TCRβV antibody molecules and the corresponding TCRβV subfamilies recognized by said anti-TCRβV antibody molecules are disclosed in Table 10A.

The various TCRβV subfamilies and/or subfamily members can be expressed at different levels in individuals, e.g., healthy individuals, as disclosed in Kitaura K. et al (2016), BMC Immunology vol 17: 38, the entire contents of which are hereby incorporated by reference. For example, TCRβ V6-5 is represented in approximately 3-6% healthy donors.

The representation of various TCRβV subfamilies and/or subfamily members can also be different in cancer cells. For example, TCRβV is present in about 3-6% of tumor infiltrating T cells irrespective of tumor type (see Li B. et al., Nature Genetics, 2016, vol: 48(7):725-32 the entire contents of which are hereby incorporated by references). Li et al., also disclose that TCRβ V6-5 is present at a high frequency in tumor cells.

### Anti-TCRβ V6 Antibodies

In one aspect, provided herein is an anti-TCRβV antibody molecule that binds to human TCRβ V6, e.g., a TCRβ V6 subfamily comprising: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments the TCRβ V6 subfamily comprises TCRβ V6-5*01 or a variant thereof. In some embodiments, TCRβ V6 comprises TCRβ V6-4*01, or a variant thereof. In some embodiments, TCRβ V6 comprises TCRβ V6-4*02, or a variant thereof. In some embodiments, TCRβ V6 comprises TCRβ V6-9*01, or a variant thereof. In some embodiments, TCRβ V6 comprises TCRβ V6-8*01, or a variant thereof. In some embodiments, TCRβ V6 comprises TCRβ V6-5*01, or a variant thereof. In some embodiments. TCRβ V6 comprises TCRβ V6-6*02, or a variant thereof. In some embodiments, TCRβ V6 comprises TCRβ V6-6*01, or a variant thereof. In some embodiments, TCRβ V6 comprises TCRβ V6-2*01, or a variant thereof. In some embodiments, TCRβ V6 comprises TCRβ V6-3*01, or a variant thereof. In some embodiments, TCRβ V6 comprises TCRβ V6-1*01, or a variant thereof.

In some embodiments, TCRβ V6-5*01 is encoded by the nucleic acid sequence of SEQ ID NO: 43, or a sequence having 85%, 90%, 95%, 99% or more identity thereof. In some embodiments, TCRβ V6-5*01 comprises the amino acid sequence of SEQ ID NO: 44, or an amino acid sequence having 85%, 90%, 95%, 99% or more identity thereof.

In some embodiments, the anti-TCRβV antibody molecule, e.g., anti-TCRβ V6 (e.g., anti-TCRβ V6-5*01) antibody molecule, is a non-murine antibody molecule, e.g., a human or humanized antibody molecule. In some embodiments, the anti-TCRβV antibody molecule, e.g., anti-TCRβ V6 (e.g., anti-TCRβ V6-5*01) antibody molecule is a human antibody molecule. In some embodiments, the anti-TCRβV antibody molecule, e.g., anti-TCRβ V6 (e.g., anti-TCRβ V6-5*01) antibody molecule is a humanized antibody molecule.

In some embodiments, the anti-TCRβV antibody molecule, e.g., anti-TCRβ V6 (e.g., anti-TCRβ V6-5*01) antibody molecule, is isolated or recombinant.

In some embodiments, the anti-TCRβV antibody molecule, e.g., anti-TCRβ V6 (e.g., anti-TCRβ V6-5*01) antibody molecule, comprises at least one antigen-binding region, e.g., a variable region or an antigen-binding fragment thereof, from an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, comprises at least one, two, three or four variable regions from an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, comprises at least one or two heavy chain variable regions from an antibody described herein, e.g., an antibody chosen from any one of A-H.1 to A-H.85, e.g., A-H.1, A-H.2 or A-H.68, or an antibody molecule described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule comprises a heavy chain variable region (VH) having a consensus sequence of SEQ ID NO: 231 or 3290.

-Consensus VH
SEQ ID NO: 231
QVQLVQSGAEVKKPGSSVKVSCKASGH/T/G/YD/T/SFH/R/D/K/
TL/D/K/T/NW/F/T/I/Y/GYIHWVRQAPGQGLEWMGR/WV/I/FF/
S/YA/PGSGN/ST/V/Y/IK/RYNEKFKGRVTITADTSTSTAYMELSSL
RSEDTAVYYCAG/VSY/IYSY/AD/GVLDYWGQGTTVTVSS

### SEQ ID NO: 3290—Consensus VH

QVQLVQSGAEVKKPGSSVKVSCKASGX1X2FX3X4X5YIHWVRQAPGQGLEWMGX6X7X8X9GS GX10X11X12YNEKFKGRVTITADTSTSTAYMELSSLRSEDTAVYYCAX13SX14YSX15X16VLDY WGQGTTVTVSS, where-in: X1 is H or T or G or Y; X2 is D or T or S; X3 is H or R or D or K or T; X4 is L or D or K or T or N; X5 is W or F or T or I or Y or G; X6 is R or W; X7 is V or I or F; X8 is F or S or Y; X9 is A or P; X10 is N or S; X11 is T or V or Y or I; X12 is K or R; X13 is G or V; X14 is Y or I; X15 is Y or A; and X16 is D or G.

In some embodiments, the anti-TCRβV antibody molecule, e.g., anti-TCRβ V6 (e.g., anti-TCRβ V6-5*01) antibody molecule, comprises at least one or two light chain variable regions from an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule comprises a light chain variable region (VL) having a consensus sequence of SEQ ID NO: 230 or 3289.

-Consensus VL
SEQ ID NO: 230
DIQMTQSPSFLSASVGDRVTITCKASQNVG/E/A/DN/DR/KVAWY/HQ
QKPGKAPKALIYSSSHRYK/SGVPSRFSGSGSGTEFTLTISSLQPEDFA
TYFCQQFKSYPLTFGQGTKLEIK

### SEQ ID NO: 3289—Consensus VL

DIQMTQSPSFLSASVGDRVTITCKASQNVX 1X2X3VAWX4QQKPGKAPKALIYSSSHRYX5GVPS RFSGSGSGTEFTLTISSLQPEDFATYFCQQFKSYPLTFGQGTKLEIK, wherein X1 is G, E, A or D; X2 is N or D; X3 is R or K; X4 is Y or H; and X5 is K or S.

In some embodiments, the anti-TCRβV antibody molecule, e.g., anti-TCRβ V6 (e.g., anti-TCRβ V6-5*01) antibody molecule, comprises a heavy chain constant region for an IgG4, e.g., a human IgG4. In still another embodiment, the anti-TCRβV antibody molecule, e.g., anti-TCRβ V6 (e.g., anti-TCRβ V6-5*01) antibody molecule includes a heavy chain constant region for an IgG1, e.g., a human IgG1. In some embodiments, the heavy chain constant region comprises an amino sequence set forth in Table 3, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V6 (e.g., anti-TCRβ V6-5*01) antibody molecule, includes a kappa light chain constant region, e.g., a human kappa light chain constant region. In some embodiments, the light chain constant region comprises an amino sequence set forth in Table 3, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V6 (e.g., anti-TCRβ V6-5*01) antibody molecule, includes at least one, two, or three complementarity determining regions (CDRs) from a heavy chain variable region (VH) of an antibody described herein. e.g., an antibody chosen from any one of A-H.1 to A-H.85, e.g., A-H.1, A-H.2 or A-H.68, or an antibody described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, includes at least one, two, or three CDRs (or collectively all of the CDRs) from a heavy chain variable region comprising an amino acid sequence shown in Table 1, or encoded by a nucleotide sequence shown in Table 1. In some embodiments, one or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1.

In some embodiments, the anti-TCRβV antibody molecule, e.g., anti-TCRβ V6 (e.g., anti-TCRβ V6-5*01) antibody molecule, includes at least one, two, or three complementarity determining regions (CDRs) from a light chain variable region of an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, includes at least one, two, or three CDRs (or collectively all of the CDRs) from a light chain variable region comprising an amino acid sequence shown in Table 1, or encoded by a nucleotide sequence shown in Table 1. In some embodiments, one or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1.

In some embodiments, the anti-TCRβV antibody molecule, e.g., anti-TCRβ V6 (e.g., anti-TCRβ V6-5*01) antibody molecule, includes at least one, two, three, four, five or six CDRs (or collectively all of the CDRs) from a heavy and light chain variable region comprising an amino acid sequence shown in Table 1, or encoded by a nucleotide sequence shown in Table 1. In some embodiments, one or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1.

In some embodiments, the anti-TCRβV antibody molecule, e.g., anti-TCRβ V6 (e.g., anti-TCRβ V6-5*01) antibody molecule, molecule includes all six CDRs from an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1, or closely related CDRs, e.g., CDRs which are identical or which have at least one amino acid alteration, but not more than two, three or four alterations (e.g., substitutions, deletions, or insertions, e.g., conservative substitutions). In some embodiments, the anti-TCRβV antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule includes at least one, two, or three CDRs according to Kabat et al. (e.g., at least one, two, or three CDRs according to the Kabat definition as set out in Table 1) from a heavy chain variable region of an antibody described herein, e.g., an antibody chosen from any one of A-H.1 to A-H.85, e.g., A-H.1, A-H.2 or A-H.68, or an antibody described in Table 1, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 1.

In some embodiments, the anti-TCRβV antibody molecule, e.g., anti-TCRβ V6 (e.g., anti-TCRβ V6-5*01) antibody molecule includes at least one, two, or three CDRs according to Kabat et al. (e.g., at least one, two, or three CDRs according to the Kabat definition as set out in Table 1) from a light chain variable region of an antibody described herein, e.g., an antibody chosen from any one of A-H.1 to A-H.85, e.g., A-H.1, A-H.2 or A-H.68, or an antibody described in Table 1, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 1.

In some embodiments, the anti-TCRβV antibody molecule, e.g., anti-TCRβ V6 (e.g., anti-TCRβ V6-5*01) antibody molecule, includes at least one, two, three, four, five, or six CDRs according to Kabat et al. (e.g., at least one, two, three, four, five, or six CDRs according to the Kabat definition as set out in Table 1) from the heavy and light chain variable regions of an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Kabat et al. shown in Table 1.

In some embodiments, the anti-TCRβV antibody molecule, e.g., anti-TCRβ V6 (e.g., anti-TCRβ V6-5*01) antibody molecule, includes all six CDRs according to Kabat et al. (e.g., all six CDRs according to the Kabat definition as set out in Table 1) from the heavy and light chain variable regions of an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions. e.g., conservative substitutions) relative to all six CDRs according to Kabat et al. shown in Table 1. In some embodiments, the anti-TCRβV antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule, includes at least one, two, or three hypervariable loops that have the same canonical structures as the corresponding hypervariable loop of an antibody described herein, e.g., an antibody chosen from chosen from any one of A-H.1 to A-H.85, e.g., A-H.1, A-H.2 or A-H.68, e.g., the same canonical structures as at least loop 1 and/or loop 2 of the heavy and/or light chain variable domains of an antibody described herein. See, e.g., Chothia et al., (1992) J. Mol. Biol. 227:799-817; Tomlinson et al., (1992) J. Mol. Biol. 227:776-798 for descriptions of hypervariable loop canonical structures. These structures can be determined by inspection of the tables described in these references.

In some embodiments, the anti-TCRβV antibody molecule, e.g., anti-TCRβ V6 (e.g., anti-TCRβ V6-5*01) antibody molecule includes at least one, two, or three CDRs according to Chothia et al. (e.g., at least one, two, or three CDRs according to the Chothia definition as set out in Table 1) from a heavy chain variable region of an antibody described herein, e.g., an antibody chosen from any one of A-H.1 to A-H.85, e.g., A-H.1, A-H.2 or A-H.68, or as described in Table 1, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 1.

In some embodiments, the anti-TCRβV antibody molecule, e.g., anti-TCRβ V6 (e.g., anti-TCRβ V6-5*01) antibody molecule includes at least one, two, or three CDRs according to Chothia et al. (e.g., at least one, two, or three CDRs according to the Chothia definition as set out in Table 1) from a light chain variable region of an antibody described herein, e.g., an antibody chosen from any one of A-H.1 to A-H.85, e.g., A-H.1, A-H.2 or A-H.68, or an antibody described in Table 1, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 1.

In some embodiments, the anti-TCRβV antibody molecule, e.g., anti-TCRβ V6 (e.g., anti-TCRβ V6-5*01) antibody molecule, includes at least one, two, three, four, five, or six CDRs according to Chothia et al. (e.g., at least one, two, three, four, five, or six CDRs according to the Chothia definition as set out in Table 1) from the heavy and light chain variable regions of an antibody described herein, e.g., an antibody chosen from any one of A-H.1 to A-H.85, e.g., A-H.1, A-H.2 or A-H.68, or an antibody described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Chothia et al. shown in Table 1.

In some embodiments, the anti-TCRβV antibody molecule, e.g., anti-TCRβ V6 (e.g., anti-TCRβ V6-5*01) antibody molecule, includes all six CDRs according to Chothia et al. (e.g., all six CDRs according to the Chothia definition as set out in Table 1) from the heavy and light chain variable regions of an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Chothia et al. shown in Table 1. In some embodiments, the anti-TCRβV antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule, molecule includes a combination of CDRs or hypervariable loops defined according to Kabat et al., Chothia et al., or as described in Table 1.

In some embodiments, the anti-TCRβV antibody molecule, e.g., anti-TCRβ V6 (e.g., anti-TCRβ V6-5*01) antibody molecule, can contain any combination of CDRs or hypervariable loops according to the Kabat and Chothia definitions.

In some embodiments, a combined CDR as set out in Table 1 is a CDR that comprises a Kabat CDR and a Chothia CDR.

In some embodiments, the anti-TCRβV antibody molecule, e.g., anti-TCRβ V6 (e.g., anti-TCRβ V6-5*01) antibody molecule, molecule includes a combination of CDRs or hypervariable loops identified as combined CDRs in Table 1. In some embodiments, the anti-TCRβV antibody molecule, e.g., anti-TCRβ V6 (e.g., anti-TCRβ V6-5*01) antibody molecule, can contain any combination of CDRs or hypervariable loops according the “combined” CDRs are described in Table 1.

In some embodiments, e.g., an embodiment comprising a variable region, a CDR (e.g., a combined CDR, Chothia CDR or Kabat CDR), or other sequence referred to herein, e.g., in Table 1, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments the antibody molecule comprises a multispecific molecule, e.g., a bispecific molecule, e.g., as described herein.

In some embodiments, the anti-TCRβV antibody molecule, e.g., anti-TCRβ V6 (e.g., anti-TCRβ V6-5*01) antibody molecule includes: (i) one, two or all of a light chain complementarity determining region 1 (LC CDR1), a light chain complementarity determining region 2 (LC CDR2), and a light chain complementarity determining region 3 (LC CDR3) of SEQ ID NO: 2, SEQ ID NO: 10 or SEQ ID NO: 11, and/or (ii) one, two or all of a heavy chain complementarity determining region 1 (HC CDR1), heavy chain complementarity determining region 2 (HC CDR2), and a heavy chain complementarity determining region 3 (HC CDR3) of SEQ ID NO: 1 or SEQ ID NO: 9.

In some embodiments, the anti-TCRβV antibody molecule, e.g., anti-TCRβ V6 (e.g., anti-TCRβ V6-5*01) antibody molecule comprises a LC CDR1, LC CDR2, and LC CDR3 of SEQ ID NO: 2, and a HC CDR1, HC CDR2, and HC CDR3 of SEQ ID NO: 1.

In some embodiments the anti-TCRβV antibody molecule, e.g., anti-TCRβ V6 (e.g., anti-TCRβ V6-5*01) antibody molecule comprises a LC CDR1, LC CDR2, and LC CDR3 of SEQ ID NO: 10, and a HC CDR1, HC CDR2, and HC CDR3 of SEQ ID NO: 9.

In some embodiments, the anti-TCRβV antibody molecule, e.g., anti-TCRβ V6 (e.g., anti-TCRβ V6-5*01) antibody molecule comprises a LC CDR1, LC CDR2, and LC CDR3 of SEQ ID NO: 11, and a HC CDR1, HC CDR2, and HC CDR3 of SEQ ID NO: 9.

In some embodiments, the anti-TCRβV antibody molecule, e.g., anti-TCRβ V6 (e.g., anti-TCRβ V6-5*01) antibody molecule comprises: (i) a LC CDR1 amino acid sequence of SEQ ID NO: 6, a LC CDR2 amino acid sequence of SEQ ID NO: 7, or a LC CDR3 amino acid sequence of SEQ ID NO: 8; and/or (ii) a HC CDR1 amino acid sequence of SEQ ID NO: 3, a HC CDR2 amino acid sequence of SEQ ID NO: 4, or a HC CDR3 amino acid sequence of SEQ ID NO: 5.

In some embodiments, the anti-TCRβV antibody molecule, e.g., anti-TCRβ V6 (e.g., anti-TCRβ V6-5*01) antibody molecule comprises: (i) a light chain variable region (VL) comprising a LC CDR1 amino acid sequence of SEQ ID NO: 6, a LC CDR2 amino acid sequence of SEQ ID NO: 7, or a LC CDR3 amino acid sequence of SEQ ID NO: 8; and/or (ii) a heavy chain variable region (VH) comprising a HC CDR1 amino acid sequence of SEQ ID NO: 3, a HC CDR2 amino acid sequence of SEQ ID NO: 4, or a HC CDR3 amino acid sequence of SEQ ID NO: 5.

In some embodiments, the anti-TCRβV antibody molecule, e.g., anti-TCRβ V6 (e.g., anti-TCRβ V6-5*01) antibody molecule comprises: (i) a LC CDR1 amino acid sequence of SEQ ID NO: 51, a LC CDR2 amino acid sequence of SEQ ID NO: 52, or a LC CDR3 amino acid sequence of SEQ ID NO: 53; and/or (ii) a HC CDR1 amino acid sequence of SEQ ID NO: 45, a HC CDR2 amino acid sequence of SEQ ID NO: 46, or a HC CDR3 amino acid sequence of SEQ ID NO: 47.

In some embodiments, the anti-TCRβV antibody molecule, e.g., anti-TCRβ V6 (e.g., anti-TCRβ V6-5*01) antibody molecule comprises: (i) a light chain variable region (VL) comprising a LC CDR1 amino acid sequence of SEQ ID NO: 51, a LC CDR2 amino acid sequence of SEQ ID NO: 52, or a LC CDR3 amino acid sequence of SEQ ID NO: 53; and/or (ii) a heavy chain variable region (VH) comprising a HC CDR1 amino acid sequence of SEQ ID NO: 45, a HC CDR2 amino acid sequence of SEQ ID NO: 46, or a HC CDR3 amino acid sequence of SEQ ID NO: 47.

In some embodiments, the anti-TCRβV antibody molecule, e.g., anti-TCRβ V6 (e.g., anti-TCRβ V6-5*01) antibody molecule comprises: (i) a LC CDR1 amino acid sequence of SEQ ID NO: 54, a LC CDR2 amino acid sequence of SEQ ID NO: 55, or a LC CDR3 amino acid sequence of SEQ ID NO: 56; and/or (ii) a HC CDR1 amino acid sequence of SEQ ID NO: 48, a HC CDR2 amino acid sequence of SEQ ID NO: 49, or a HC CDR3 amino acid sequence of SEQ ID NO: 50.

In some embodiments, the anti-TCRβV antibody molecule, e.g., anti-TCRβ V6 (e.g., anti-TCRβ V6-5*01) antibody molecule comprises: (i) a light chain variable region (VL) comprising a LC CDR1 amino acid sequence of SEQ ID NO: 54, a LC CDR2 amino acid sequence of SEQ ID NO: 55, or a LC CDR3 amino acid sequence of SEQ ID NO: 56; and/or (ii) a heavy chain variable region (VH) comprising a HC CDR1 amino acid sequence of SEQ ID NO: 48, a HC CDR2 amino acid sequence of SEQ ID NO: 49, or a HC CDR3 amino acid sequence of SEQ ID NO: 50.

In some embodiments, the anti-TCRβV antibody molecule, e.g., anti-TCRβ V6 (e.g., anti-TCRβ V6-5*01) antibody molecule comprises a VH and/or a VL of an antibody described in Table 1, or a sequence with at least 80%, 85%, 90%, 95%, 96%, 97%, 98%, 99% or more identity thereto.

In some embodiments, the anti-TCRβV antibody molecule, e.g., anti-TCRβ V6 (e.g., anti-TCRβ V6-5*01) antibody molecule comprises a VH and a VL of an antibody described in Table 1, or a sequence with at least 80%, 85%, 90%, 95%, 96%, 970, 98%, 99% or more identity thereto.

In some embodiments, an anti-TCRVb antibody as described herein has an antigen binding domain having a VL having a consensus sequence of SEQ ID NO: 230, wherein position 30 is G, E, A or D; position 31 is N or D; position 32 is R or K; position 36 is Y or H; and/or position 56 is K or S.

In some embodiments, an anti-TCRVb antibody as described herein has an antigen binding domain having a VH having a consensus sequence of SEQ ID NO: 231, wherein: position 27 is H or T or G or Y; position 28 is D or T or S; position 30 is H or R or D or K or T; position 31 is L or D or K or T or N; position 32 is W or F or T or I or Y or G; position 49 is R or W; position 50 is V or I or F; position 51 is F or S or Y; position 52 is A or P; position 56 is N or S; position 57 is T or V or Y or 1; position 58 is K or R; position 97 is G or V; position 99 is Y or I; position 102 is Y or A; and/or position 103 is D or G.

Exemplary anti-TCRβ V6 antibodies are provided in Table 16. In some embodiments, the anti-TCRβ V6 is antibody 0, as provided in Table 16. In some embodiments, the anti-TCRβV antibody comprises one or more (e.g., all three) of a LC CDR1, LC CDR2, and LC CDR3 provided in Table 16; and/or one or more (e.g., all three) of a HC CDR1, HC CDR2, and HC CDR3 provided in Table 16, or a sequence with at least 95% sequence identity thereto. In some embodiments, antibody comprises a variable heavy chain (VH) and/or a variable light chain (VL) provided in Table 16, or a sequence with at least 95% sequence identity thereto.

In some embodiments, the anti-TCRβ V6 antibody molecule comprises a VH and/or a VL of an antibody described in Table 16, or a sequence with at least 80%, 85%, 90%, 95%, 96%, 97%, 98%, 99% or more identity thereto.

In some embodiments, the anti-TCRβ V6 antibody molecule comprises a VH and a VL of an antibody described in Table 16, or a sequence with at least 80%, 85%, 90%, 95%, 96%, 97%, 98%, 99% or more identity thereto.

### Anti-TCRβ V5 Antibodies

In one aspect, provided herein is an anti-TCRβV antibody molecule that binds to human TCRβ V5. In some embodiments, the TCRβ V5 subfamily comprises TCRβ V5-5*01, TCRβ V5-6*01, TCRβ V5-4*01, TCRβ V5-8*01, TCRβ V5-1*01, or a variant thereof.

In some embodiments, the anti-TCRβV antibody molecule, e.g., anti-TCRβ V5 (e.g., anti-TCRβ V5-1*01) antibody molecule, is a non-murine antibody molecule, e.g., a human or humanized antibody molecule. In some embodiments, the anti-TCRβV antibody molecule, e.g., anti-TCRβ V5 (e.g., anti-TCRs V-1*01) antibody molecule is a human antibody molecule. In some embodiments, the anti-TCRβV antibody molecule, e.g., anti-TCRβ V5 (e.g., anti-TCRβ V5-1*01) antibody molecule is a humanized antibody molecule.

In some embodiments, the anti-TCRβV antibody molecule, e.g., anti-TCRβ V5 (e.g., anti-TCRβ V5-1*01) antibody molecule, is isolated or recombinant.

Exemplary anti-TCRβ V5 antibodies are provided in Table 10B. In some embodiments, the anti-TCRβ V5 is an antibody C, e.g., humanized antibody C (antibody C-H), as provided in Table 10B. In some embodiments, the anti-TCRβV antibody comprises one or more (e.g., all three) of a LC CDR1, LC CDR2, and LC CDR3 provided in Table 10B; and/or one or more (e.g., all three) of a HC CDR1, HC CDR2, and HC CDR3 provided in Table 10B, or a sequence with at least 95% sequence identity thereto. In some embodiments, antibody C comprises a variable heavy chain (VH) and/or a variable light chain (VL) provided in Table 10B, or a sequence with at least 95% sequence identity thereto.

Exemplary anti-TCRβ V5 antibodies are provided in Table 10C. In some embodiments, the anti-TCRβ V5 is antibody N, as provided in Table 10C. In some embodiments, the anti-TCRβV antibody comprises one or more (e.g., all three) of a LC CDR1, LC CDR2, and LC CDR3 provided in Table 10C; and/or one or more (e.g., all three) of a HC CDR1, HC CDR2, and HC CDR3 provided in Table 10C, or a sequence with at least 95% sequence identity thereto. In some embodiments, antibody comprises a variable heavy chain (VH) and/or a variable light chain (VL) provided in Table 10C, or a sequence with at least 95% sequence identity thereto.

Exemplary anti-TCRβ V5 antibodies are provided in Table 11. In some embodiments, the anti-TCRβ V5 is antibody E, e.g., humanized antibody E (antibody E-H), as provided in Table 11. In some embodiments, the anti-TCRβV antibody comprises one or more (e.g., all three) of a LC CDR1, LC CDR2, and LC CDR3 provided in Table 11; and/or one or more (e.g., all three) of a HC CDR1, HC CDR2, and HC CDR3 provided in Table 11, or a sequence with at least 95% sequence identity thereto. In some embodiments, antibody E comprises a variable heavy chain (VH) and/or a variable light chain (VL) provided in Table 11, or a sequence with at least 95% sequence identity thereto.

In some embodiments, antibody E comprises a heavy chain comprising the amino acid sequence of SEQ ID NO: 3284 and/or a light chain comprising the amino acid sequence of SEQ ID NO: 3285, or a sequence with at least 95% sequence identity thereto.

In some embodiments, the anti-TCRβ V5 antibody molecule comprises a VH and/or a VL of an antibody described in Table 10B, or a sequence with at least 80%, 85%, 90%, 95%, 96%, 97%, 98%, 99% or more identity thereto.

In some embodiments, the anti-TCRβ V5 antibody molecule comprises a VH and a VL of an antibody described in Table 10B, or a sequence with at least 80%, 85%, 90%, 95%, 96%, 97%, 98%, 99% or more identity thereto.

In some embodiments, the anti-TCRβ V5 antibody molecule comprises a VH and/or a VL of an antibody described in Table 10C, or a sequence with at least 80%, 85%, 90%, 95%, 96%, 97%, 98%, 99% or more identity thereto.

In some embodiments, the anti-TCRβ V5 antibody molecule comprises a VH and a VL of an antibody described in Table 10C, or a sequence with at least 80%, 85%, 90%, 95%, 96%, 97%, 98%, 99% or more identity thereto.

In some embodiments, the anti-TCRβ V5 antibody molecule comprises a VH and/or a VL of an antibody described in Table 11, or a sequence with at least 80%, 85%, 90%, 95%, 96%, 97%, 98%, 99% or more identity thereto.

In some embodiments, the anti-TCRβ V5 antibody molecule comprises a VH and a VL of an antibody described in Table 11, or a sequence with at least 80%, 85%, 90%, 95%, 96%, 97%, 98%, 99% or more identity thereto.

### Anti-TCRβ V12 Antibodies

In one aspect, provided herein is an anti-TCRβV antibody molecule that binds to human TCRβ V12, e.g., a TCRβ V12 subfamily comprising: TCRβ V12-4*01, TCRβ V12-3*01 or TCRβ V12-5*01. In some embodiments the TCRβ V12 subfamily comprises TCRβ V12-4*01. In some embodiments the TCRβ V12 subfamily comprises TCRβ V12-3*01.

In some embodiments, the anti-TCRβV antibody molecule, e.g., anti-TCRβ V12 antibody molecule, is a non-murine antibody molecule, e.g., a human or humanized antibody molecule. In some embodiments, the anti-TCRβV antibody molecule, e.g., anti-TCRβ V12 antibody molecule is a human antibody molecule. In some embodiments, the anti-TCRβV antibody molecule, e.g., anti-TCRβ V12 antibody molecule is a humanized antibody molecule.

In some embodiments, the anti-TCRβV antibody molecule, e.g., anti-TCRβ V12 antibody molecule, is isolated or recombinant.

In some embodiments, the anti-TCRβV antibody molecule, e.g., anti-TCRβ V12 antibody molecule, comprises at least one antigen-binding region, e.g., a variable region or an antigen-binding fragment thereof, from an antibody described herein, e.g., an antibody described in Table 2, or encoded by a nucleotide sequence in Table 2, or a sequence substantially identical (e.g., at least 80%, 85%, 90%, 92%, 95%, 970, 98%, 99% or higher identical) to any of the aforesaid sequences.

In some embodiments, the anti-TCRβV antibody molecule, e.g., anti-TCRβ V12 antibody molecule, comprises at least one, two, three or four variable regions from an antibody described herein, e.g., an antibody as described in Table 2, or encoded by a nucleotide sequence in Table 2, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V2 antibody molecule, comprises at least one or two heavy chain variable regions from an antibody described herein, e.g., an antibody as described in Table 2, or encoded by a nucleotide sequence in Table 2, or a sequence substantially identical (e.g., at least 80%, 85%, 90%, 92%, 95%, 97%, 98%, 99/a or higher identical) to any of the aforesaid sequences.

In some embodiments, the anti-TCRβV antibody molecule, e.g., anti-TCRβ V12 antibody molecule, comprises at least one or two light chain variable regions from an antibody described herein, e.g., an antibody as described in Table 2, or encoded by a nucleotide sequence in Table 2, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, comprises a heavy chain constant region for an IgG4, e.g., a human IgG4. In still another embodiment, the anti-TCRβV antibody molecule, e.g., anti-TCRβ V12 antibody molecule, includes a heavy chain constant region for an IgG1, e.g., a human IgG1. In some embodiments, the heavy chain constant region comprises an amino sequence set forth in Table 3, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V12 antibody molecule, includes a kappa light chain constant region, e.g., a human kappa light chain constant region. In some embodiments, the light chain constant region comprises an amino sequence set forth in Table 3, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V12 antibody molecule, includes at least one, two, or three complementarity determining regions (CDRs) from a heavy chain variable region of an antibody described herein, e.g., an antibody as described in Table 2, or encoded by the nucleotide sequence in Table 2, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, includes at least one, two, or three CDRs (or collectively all of the CDRs) from a heavy chain variable region comprising an amino acid sequence shown in Table 2, or encoded by a nucleotide sequence shown in Table 2. In some embodiments, one or more of the CDRs (or collectively all of the CDRs) have one, two, three, four, five, six or more changes, e.g., amino acid substitutions or deletions, relative to the amino acid sequence shown in Table 2, or encoded by a nucleotide sequence shown in Table 2.

In some embodiments, the anti-TCRβV antibody molecule, e.g., anti-TCRβ V12 antibody molecule, includes at least one, two, or three complementarity determining regions (CDRs) from a light chain variable region of an antibody described herein, e.g., an antibody as described in Table 2, or encoded by the nucleotide sequence in Table 2, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, includes at least one, two, or three CDRs (or collectively all of the CDRs) from a light chain variable region comprising an amino acid sequence shown in Table 2, or encoded by a nucleotide sequence shown in Table 2. In some embodiments, one or more of the CDRs (or collectively all of the CDRs) have one, two, three, four, five, six or more changes, e.g., amino acid substitutions or deletions, relative to the amino acid sequence shown in Table 2, or encoded by a nucleotide sequence shown in Table 2.

In some embodiments, the anti-TCRβV antibody molecule, e.g., anti-TCRβ V12 antibody molecule, includes at least one, two, three, four, five or six CDRs (or collectively all of the CDRs) from a heavy and light chain variable region comprising an amino acid sequence shown in Table 2, or encoded by a nucleotide sequence shown in Table 2. In some embodiments, one or more of the CDRs (or collectively all of the CDRs) have one, two, three, four, five, six or more changes, e.g., amino acid substitutions or deletions, relative to the amino acid sequence shown in Table 2, or encoded by a nucleotide sequence shown in Table 2.

In some embodiments, the anti-TCRβV antibody molecule, e.g., anti-TCRβ V12 antibody molecule, molecule includes all six CDRs from an antibody described herein, e.g., an antibody as described in Table 2, or encoded by the nucleotide sequence in Table 2, or closely related CDRs, e.g., CDRs which are identical or which have at least one amino acid alteration, but not more than two, three or four alterations (e.g., substitutions, deletions, or insertions, e.g., conservative substitutions). In some embodiments, the anti-TCRβV antibody molecule, e.g., anti-TCRβ V12 antibody molecule, may include any CDR described herein.

In some embodiments, the anti-TCRβV antibody molecule, e.g., anti-TCRβ V12 antibody molecule includes at least one, two, or three CDRs according to Kabat et al. (e.g., at least one, two, or three CDRs according to the Kabat definition as set out in Table 2) from a heavy chain variable region of an antibody described herein, e.g., an antibody chosen as described in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 2.

In some embodiments, the anti-TCRβV antibody molecule, e.g., anti-TCRβ V12 antibody molecule includes at least one, two, or three CDRs according to Kabat et al. (e.g., at least one, two, or three CDRs according to the Kabat definition as set out in Table 2) from a light chain variable region of an antibody described herein, e.g., an antibody as described in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 2.

In some embodiments, the anti-TCRβV antibody molecule, e.g., anti-TCRβ V12 antibody molecule includes at least one, two, three, four, five, or six CDRs according to Kabat et al. (e.g., at least one, two, three, four, five, or six CDRs according to the Kabat definition as set out in Table 2) from the heavy and light chain variable regions of an antibody described herein, e.g., an antibody as described in Table 2, or encoded by the nucleotide sequence in Table 2; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Kabat et al. shown in Table 2.

In some embodiments, the anti-TCRβV antibody molecule, e.g., anti-TCRβ V12 antibody molecule includes all six CDRs according to Kabat et al. (e.g., all six CDRs according to the Kabat definition as set out in Table 2) from the heavy and light chain variable regions of an antibody described herein, e.g., an antibody as described in Table 2, or encoded by the nucleotide sequence in Table 2; or encoded by the nucleotide sequence in Table 2; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Kabat et al. shown in Table 2. In some embodiments, the anti-TCRβV antibody molecule, e.g., anti-TCRβ V12 antibody molecule may include any CDR described herein.

In some embodiments, the anti-TCRβV antibody molecule, e.g., anti-TCRβ V12 antibody molecule includes at least one, two, or three hypervariable loops that have the same canonical structures as the corresponding hypervariable loop of an antibody described herein, e.g., an antibody described in Table 2, e.g., the same canonical structures as at least loop 1 and/or loop 2 of the heavy and/or light chain variable domains of an antibody described herein. See, e.g., Chothia et al., (1992) J. Mol. Biol. 227:799-817; Tomlinson et al., (1992) J. Mol. Biol. 227:776-798 for descriptions of hypervariable loop canonical structures. These structures can be determined by inspection of the tables described in these references.

In some embodiments, the anti-TCRβV antibody molecule, e.g., anti-TCRβ V12 antibody molecule includes at least one, two, or three CDRs according to Chothia et al. (e.g., at least one, two, or three CDRs according to the Chothia definition as set out in Table 2) from a heavy chain variable region of an antibody described herein, e.g., an antibody chosen as described in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 2.

In some embodiments, the anti-TCRβV antibody molecule, e.g., anti-TCRβ V12 antibody molecule includes at least one, two, or three CDRs according to Chothia et al. (e.g., at least one, two, or three CDRs according to the Chothia definition as set out in Table 2) from a light chain variable region of an antibody described herein, e.g., an antibody as described in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions. e.g., conservative substitutions) relative to one, two, or three CDRs according to Chothia et al. shown in Table 2.

In some embodiments, the anti-TCRβV antibody molecule, e.g., anti-TCRβ V12 antibody molecule includes at least one, two, three, four, five, or six CDRs according to Chothia et al. (e.g., at least one, two, three, four, five, or six CDRs according to the Chothia definition as set out in Table 2) from the heavy and light chain variable regions of an antibody described herein, e.g., an antibody as described in Table 2, or encoded by the nucleotide sequence in Table 2; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Chothia et al. shown in Table 2.

In some embodiments, the anti-TCRβV antibody molecule, e.g., anti-TCRβ V12 antibody molecule includes all six CDRs according to Chothia et al. (e.g., all six CDRs according to the Chothia definition as set out in Table 2) from the heavy and light chain variable regions of an antibody described herein, e.g., an antibody as described in Table 2, or encoded by the nucleotide sequence in Table 2; or encoded by the nucleotide sequence in Table 2; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Chothia et al. shown in Table 2. In some embodiments, the anti-TCRβV antibody molecule, e.g., anti-TCRβ V12 antibody molecule may include any CDR described herein.

In some embodiments, the anti-TCRβV antibody molecule, e.g., anti-TCRβ V12 antibody molecule includes at least one, two, or three CDRs according to a combined CDR (e.g., at least one, two, or three CDRs according to the combined CDR definition as set out in Table 2) from a heavy chain variable region of an antibody described herein, e.g., an antibody chosen as described in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to combined CDR shown in Table 2.

In some embodiments, the anti-TCRβV antibody molecule, e.g., anti-TCRβ V12 antibody molecule includes at least one, two, or three CDRs according to a combined CDR (e.g., at least one, two, or three CDRs according to the combined CDR definition as set out in Table 2) from a light chain variable region of an antibody described herein, e.g., an antibody as described in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to a combined CDR shown in Table 2.

In some embodiments, the anti-TCRβV antibody molecule, e.g., anti-TCRβ V12 antibody molecule includes at least one, two, three, four, five, or six CDRs according to a combined CDR. (e.g., at least one, two, three, four, five, or six CDRs according to the combined CDR definition as set out in Table 2) from the heavy and light chain variable regions of an antibody described herein, e.g., an antibody as described in Table 2, or encoded by the nucleotide sequence in Table 2; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to a combined CDR shown in Table 2.

In some embodiments, the anti-TCRβV antibody molecule, e.g., anti-TCRβ V12 antibody molecule includes all six CDRs according to a combined CDR (e.g., all six CDRs according to the combined CDR definition as set out in Table 2) from the heavy and light chain variable regions of an antibody described herein, e.g., an antibody as described in Table 2, or encoded by the nucleotide sequence in Table 2; or encoded by the nucleotide sequence in Table 2; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to a combined CDR shown in Table 2. In some embodiments, the anti-TCRβV antibody molecule, e.g., anti-TCRβ V12 antibody molecule may include any CDR described herein.

In some embodiments, a combined CDR as set out in Table 1 is a CDR that comprises a Kabat CDR and a Chothia CDR.

In some embodiments, the anti-TCRβV antibody molecule, e e.g., anti-TCRβ V12 antibody molecule, molecule includes a combination of CDRs or hypervariable loops identified as combined CDRs in Table 1. In some embodiments, the anti-TCRβV antibody molecule, e.g., anti-TCRβ V12 antibody molecule, can contain any combination of CDRs or hypervariable loops according the “combined” CDRs are described in Table 1.

In some embodiments, the anti-TCRβV antibody molecule, e.g., anti-TCRβ V12 antibody molecule includes a combination of CDRs or hypervariable loops defined according to the Kabat et al, and Chothia et al., or as described in Table 1.

In some embodiments, the anti-TCRβV antibody molecule, e.g., anti-TCRβ V12 antibody molecule can contain any combination of CDRs or hypervariable loops according to the Kabat and Chothia definitions.

In some embodiments, e.g., an embodiment comprising a variable region, a CDR (e.g., a combined CDR, Chothia CDR or Kabat CDR), or other sequence referred to herein, e.g., in Table 2, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments the antibody molecule comprises a multispecific molecule, e.g., a bispecific molecule, e.g., as described herein.

In some embodiments, the anti-TCRβV antibody molecule, e.g., anti-TCRβ V12 antibody molecule includes: (i) one, two or all of a light chain complementarity determining region 1 (LC CDR1), a light chain complementarity determining region 2 (LC CDR2), and a light chain complementarity determining region 3 (LC CDR3) of SEQ ID NO: 16, SEQ ID NO: 26, SEQ ID NO: 27, SEQ ID NO: 28, SEQ ID NO: 29 or SEQ ID NO: 30, and/or (ii) one, two or all of a heavy chain complementarity determining region 1 (HC CDR1), heavy chain complementarity determining region 2 (HC CDR2), and a heavy chain complementarity determining region 3 (HC CDR3) of SEQ ID NO: 15, SEQ ID NO: 23, SEQ ID NO: 24 or SEQ ID NO: 25.

In some embodiments, the anti-TCRβV antibody molecule, e.g., anti-TCRβ V12 antibody molecule comprises: (i) a LC CDR1 amino acid sequence of SEQ ID NO: 20, a LC CDR2 amino acid sequence of SEQ ID NO: 21, or a LC CDR3 amino acid sequence of SEQ ID NO: 22, and/or (ii) a HC CDR1 amino acid sequence of SEQ ID NO: 17, a HC CDR2 amino acid sequence of SEQ ID NO: 18, or a HC CDR3 amino acid sequence of SEQ ID NO: 19.

In some embodiments, the anti-TCRβV antibody molecule, e.g., anti-TCRβ V12 antibody molecule comprises: (i) a light chain variable region (VL) comprising a LC CDR1 amino acid sequence of SEQ ID NO: 20, a LC CDR2 amino acid sequence of SEQ ID NO: 21, and a LC CDR3 amino acid sequence of SEQ ID NO: 2; and/or (ii) a heavy chain variable region (VH) comprising a HC CDR1 amino acid sequence of SEQ ID NO: 17, a HC CDR2 amino acid sequence of SEQ ID NO: 18, and a HC CDR3 amino acid sequence of SEQ ID NO: 19.

In some embodiments, the anti-TCRβV antibody molecule, e.g., anti-TCRβ V12 antibody molecule comprises: (i) a LC CDR1 amino acid sequence of SEQ ID NO: 63, a LC CDR2 amino acid sequence of SEQ ID NO: 64, or a LC CDR3 amino acid sequence of SEQ ID NO: 65; and/or (ii) a HC CDR1 amino acid sequence of SEQ ID NO: 57, a HC CDR2 amino acid sequence of SEQ ID NO: 58, or a HC CDR3 amino acid sequence of SEQ ID NO: 59.

In some embodiments, the anti-TCRβV antibody molecule, e.g., anti-TCRβ V12 antibody molecule comprises: (i) a light chain variable region (VL) comprising a LC CDR1 amino acid sequence of SEQ ID NO: 63, a LC CDR2 amino acid sequence of SEQ ID NO: 64, or a LC CDR3 amino acid sequence of SEQ ID NO: 65; and/or (ii) a heavy chain variable region (VH) comprising a HC CDR1 amino acid sequence of SEQ ID NO: 57, a HC CDR2 amino acid sequence of SEQ ID NO: 58, or a HC CDR3 amino acid sequence of SEQ ID NO: 59.

In some embodiments, the anti-TCRβV antibody molecule, e.g., anti-TCRβ V12 antibody molecule comprises: (i) a LC CDR1 amino acid sequence of SEQ ID NO: 66, a LC CDR2 amino acid sequence of SEQ ID NO: 67, or a LC CDR3 amino acid sequence of SEQ ID NO: 68; and/or (ii) a HC CDR1 amino acid sequence of SEQ ID NO: 60, a HC CDR2 amino acid sequence of SEQ ID NO: 61, or a HC CDR3 amino acid sequence of SEQ ID NO: 62.

In some embodiments, the anti-TCRβV antibody molecule, e.g., anti-TCRβ V12 antibody molecule comprises: (i) a light chain variable region (VL) comprising a LC CDR1 amino acid sequence of SEQ ID NO: 63, a LC CDR2 amino acid sequence of SEQ ID NO: 64, or a LC CDR3 amino acid sequence of SEQ ID NO: 65; and/or (ii) a heavy chain variable region (VH) comprising a HC CDR1 amino acid sequence of SEQ ID NO: 57, a HC CDR2 amino acid sequence of SEQ ID NO: 58, or a HC CDR3 amino acid sequence of SEQ ID NO: 59.

In some embodiments, the light or the heavy chain variable framework (e.g., the region encompassing at least FR1, FR2, FR3, and optionally FR4) of the anti-TCRβV antibody molecule, e.g., anti-TCRβ V12 antibody molecule can be chosen from: (a) a light or heavy chain variable framework including at least 80%, 85%, 87% 90%, 92%, 93%, 95%, 97%, 98%, or 100% of the amino acid residues from a human light or heavy chain variable framework, e.g., a light or heavy chain variable framework residue from a human mature antibody, a human germline sequence, or a human consensus sequence; (b) a light or heavy chain variable framework including from 20% to 80%, 40% to 60%, 60% to 90%, or 70% to 95% of the amino acid residues from a human light or heavy chain variable framework, e.g., a light or heavy chain variable framework residue from a human mature antibody, a human germline sequence, or a human consensus sequence; (c) a non-human framework (e.g., a rodent framework); or (d) a non-human framework that has been modified, e.g., to remove antigenic or cytotoxic determinants, e.g., deimmunized, or partially humanized. In some embodiments, the light or heavy chain variable framework region (particularly FR1, FR2 and/or FR3) includes a light or heavy chain variable framework sequence at least 70, 75, 80, 85, 87, 88, 90, 92, 94, 95, 96, 97, 98, 99% identical or identical to the frameworks of a VL or VH segment of a human germline gene.

In some embodiments, the anti-TCRβV antibody molecule, e.g., anti-TCRβ V12 antibody molecule, comprises a heavy chain variable domain having at least one, two, three, four, five, six, seven, ten, fifteen, twenty or more changes, e.g., amino acid substitutions or deletions, from an amino acid sequence described in Table 2.e.g., the amino acid sequence of the FR region in the entire variable region, e.g., SEQ ID NOs: 23-25.

Alternatively, or in combination with the heavy chain substitutions described herein the anti-TCRβV antibody molecule, e.g., anti-TCRβ V12 antibody molecule comprises a light chain variable domain having at least one, two, three, four, five, six, seven, ten, fifteen, twenty or more amino acid changes, e.g., amino acid substitutions or deletions, from an amino acid sequence of an antibody described herein. e.g., the amino acid sequence of the FR region in the entire variable region, e.g., SEQ ID NOs: 26-30.

In some embodiments, the anti-TCRβV antibody molecule, e.g., anti-TCRβ V12 antibody molecule includes one, two, three, or four heavy chain framework regions a sequence substantially identical thereto.

In some embodiments, the anti-TCRβV antibody molecule, e.g., anti-TCRβ V12 antibody molecule includes one, two, three, or four light chain framework regions or a sequence substantially identical thereto. In some embodiments, the anti-TCRβV antibody molecule, e.g., anti-TCRβ V12 antibody molecule comprises the light chain framework region 1. In some embodiments, the anti-TCRβV antibody molecule, e.g., anti-TCRβ V12 antibody molecule comprises the light chain framework region 2. In some embodiments, the anti-TCRβV antibody molecule, e.g., anti-TCRβ V12 antibody molecule comprises the light chain framework region 3. In some embodiments, the anti-TCRβV antibody molecule, e.g., anti-TCRβ V12 antibody molecule comprises the light chain framework region 4.

In some embodiments, the anti-TCRβV antibody molecule, e.g., anti-TCRβ V12 antibody molecule comprises a light chain comprising a framework region, e.g., framework region 1 (FR1), comprising a change, e.g., a substitution (e.g., a conservative substitution) at one or more, e.g., all, position as described herein according to Kabat numbering. In some embodiments, FR1 comprises an Aspartic Acid at position 1, e.g., a substitution at position 1 according to Kabat numbering, e.g., an Alanine to Aspartic Acid substitution. In some embodiments, FR1 comprises an Asparagine at position 2, e.g., a substitution at position 2 according to Kabat numbering, e.g., an Isoleucine to Asparagine substitution, Serine to Asparagine substitution or Tyrosine to Asparagine substitution. In some embodiments, FR1 comprises a Leucine at position 4, e.g., a substitution at position 4 according to Kabat numbering, e.g., a Methionine to Leucine substitution.

In some embodiments, the anti-TCRβV antibody molecule, e.g., anti-TCRβ V12 antibody molecule comprises a light chain comprising a framework region, e.g., framework region 1 (FR1), comprising a substitution at position 1 according to Kabat numbering, e.g., an Alanine to Aspartic Acid substitution, a substitution at position 2 according to Kabat numbering, e.g., an Isoleucine to Asparagine substitution, Serine to Asparagine substitution or Tyrosine to Asparagine substitution, and a substitution at position 4 according to Kabat numbering. e.g., a Methionine to Leucine substitution. In some embodiments, the anti-TCRβV antibody molecule, e.g., anti-TCRβ V12 antibody molecule comprises a light chain comprising a framework region, e.g., framework region 1 (FR1), comprising a substitution at position 1 according to Kabat numbering, e.g., an Alanine to Aspartic Acid substitution, and a substitution at position 2 according to Kabat numbering, e.g., an Isoleucine to Asparagine substitution, Serine to Asparagine substitution or Tyrosine to Asparagine substitution. In some embodiments, the anti-TCRβV antibody molecule, e.g., anti-TCRβ V12 antibody molecule comprises a light chain comprising a framework region, e.g., framework region 1 (FR1), comprising a substitution at position 1 according to Kabat numbering, e.g., an Alanine to Aspartic Acid substitution, and a substitution at position 4 according to Kabat numbering, e.g., a Methionine to Leucine substitution. In some embodiments, the anti-TCRβV antibody molecule, e.g., anti-TCRβ V12 antibody molecule comprises a light chain comprising a framework region, e.g., framework region 1 (FR1), comprising a substitution at position 2 according to Kabat numbering, e.g., an Isoleucine to Asparagine substitution, Serine to Asparagine substitution or Tyrosine to Asparagine substitution, and a substitution at position 4 according to Kabat numbering, e.g., a Methionine to Leucine substitution. In some embodiments, the substitution is relative to a human germline light chain framework region sequence.

In some embodiments, the anti-TCRβV antibody molecule, e.g., anti-TCRβ V2 antibody molecule comprises a light chain comprising a framework region, e.g., framework region 3 (FR3), comprising a change, e.g., a substitution (e.g., a conservative substitution) at one or more, e.g., all, position as described herein according to Kabat numbering. In some embodiments, FR3 comprises a Glycine at position 66, e.g., a substitution at position 66 according to Kabat numbering, e.g., a Lysine to Glycine substitution, or a Serine to Glycine substitution. In some embodiments, FR3 comprises an Asparagine at position 69, e.g., a substitution at position 69 according to Kabat numbering, e.g., a Tyrosine to Asparagine substitution. In some embodiments, FR3 comprises a Tyrosine at position 71, e.g., a substitution at position 71 according to Kabat numbering, e.g., a Phenylalanine to Tyrosine substitution, or an Alanine to Tyrosine substitution.

In some embodiments, the anti-TCRβV antibody molecule, e.g., anti-TCRβ V12 antibody molecule comprises a light chain comprising a framework region, e.g., framework region 3 (FR3), comprising a substitution at position 66 according to Kabat numbering, e.g., a Lysine to Glycine substitution, or a Serine to Glycine substitution, and a substitution at position 69 according to Kabat numbering, e.g., a Tyrosine to Asparagine substitution. In some embodiments, the anti-TCRβV antibody molecule, e.g., anti-TCRβ V12 antibody molecule comprises a light chain comprising a framework region, e.g., framework region 3 (FR3), comprising a substitution at position 66 according to Kabat numbering, e.g., Lysine to Glycine substitution, or a Serine to Glycine substitution, and a substitution at position 71 according to Kabat numbering, e.g., a Phenylalanine to Tyrosine substitution, or an Alanine to Tyrosine substitution. In some embodiments, the anti-TCRβV antibody molecule, e.g., anti-TCRβ V12 antibody molecule comprises a light chain comprising a framework region, e.g., framework region 3 (FR3), comprising a substitution at position 69 according to Kabat numbering. e.g., a Tyrosine to Asparagine substitution and a substitution at position 71 according to Kabat numbering, e.g., a Phenylalanine to Tyrosine substitution, or an Alanine to Tyrosine substitution. In some embodiments, the anti-TCRβV antibody molecule, e.g., anti-TCRβ V12 antibody molecule comprises a light chain comprising a framework region, e.g., framework region 3 (FR3), comprising a substitution at position 66 according to Kabat numbering, e.g., a Lysine to Glycine substitution, or a Serine to Glycine substitution, a substitution at position 69 according to Kabat numbering, e.g., a Tyrosine to Asparagine substitution and a substitution at position 71 according to Kabat numbering, e.g., a Phenylalanine to Tyrosine substitution, or an Alanine to Tyrosine substitution. In some embodiments, the substitution is relative to a human germline light chain framework region sequence.

In some embodiments, the anti-TCRβV antibody molecule, e.g., anti-TCRβ V12 antibody molecule comprises a light chain comprising: a framework region 1 (FR1) comprising a substitution at position 2 according to Kabat numbering, e.g., a Isoleucine to Asparagine substitution; and a framework region 3 (FR3), comprising a substitution at position 69 according to Kabat numbering, e.g., a Threonine to Asparagine substitution and a substitution at position 71 according to Kabat numbering, e.g., a Phenylalanine to Tyrosine substitution, e.g., as shown in the amino acid sequence of SEQ ID NO: 26. In some embodiments, the substitution is relative to a human germline light chain framework region sequence.

In some embodiments, the anti-TCRβV antibody molecule, e.g., anti-TCRβ V12 antibody molecule comprises a light chain comprising: (a) a framework region 1 (FR1) comprising a substitution at position 1 according to Kabat numbering. e.g., a Alanine to Aspartic Acid substitution, and a substitution at position 2 according to Kabat numbering, e.g., a Isoleucine to Asparagine substitution; and (b) a framework region 3 (FR3), comprising a substitution at position 69 according to Kabat numbering, e.g., a Threonine to Asparagine substitution and a substitution at position 71 according to Kabat numbering, e.g., a Phenylalanine to Tyrosine substitution, e.g., as shown in the amino acid sequence of SEQ ID NO: 27 In some embodiments, the substitution is relative to a human germline light chain framework region sequence.

In some embodiments, the anti-TCRβV antibody molecule, e.g., anti-TCRβ V12 antibody molecule comprises a light chain comprising: (a) a framework region 1 (FR1) comprising a substitution at position 2 according to Kabat numbering, e.g., a Serine to Asparagine substitution; and a substitution at position 4 according to Kabat numbering, e.g., a Methionine to Leucine substitution; and (b) a framework region 3 (FR3), comprising a substitution at position 69 according to Kabat numbering, e.g., a Threonine to Asparagine substitution and a substitution at position 71 according to Kabat numbering, e.g., a Phenylalanine to Tyrosine substitution, e.g., as shown in the amino acid sequence of SEQ ID NO: 28 In some embodiments, the substitution is relative to a human germline light chain framework region sequence.

In some embodiments, the anti-TCRβV antibody molecule, e.g., anti-TCRβ V12 antibody molecule comprises a light chain comprising: (a) a framework region 1 (FR1) comprising a substitution at position 2 according to Kabat numbering, e.g., a Serine to Asparagine substitution; and (b) a framework region 3 (FR3) comprising a substitution at position 66 according to Kabat numbering, e.g., a Lysine to Glycine substitution; a substitution at position 69 according to Kabat numbering, e.g., a Threonine to Asparagine substitution; and a substitution at position 71 according to Kabat numbering, e.g., a Alanine to Tyrosine substitution, e.g., as shown in the amino acid sequence of SEQ ID NO: 29. In some embodiments, the substitution is relative to a human germline light chain framework region sequence.

In some embodiments, the anti-TCRβV antibody molecule, e.g., anti-TCRβ V12 antibody molecule comprises a light chain comprising: (a) a framework region 1 (FR1) comprising a substitution at position 2 according to Kabat numbering. e.g., a Tyrosine to Asparagine substitution; and (b) a framework region 3 (FR3) comprising a substitution at position 66 according to Kabat numbering, e.g., a Serine to Glycine substitution; a substitution at position 69 according to Kabat numbering, e.g., a Threonine to Asparagine substitution; and a substitution at position 71 according to Kabat numbering, e.g., a Alanine to Tyrosine substitution, e.g., as shown in the amino acid sequence of SEQ ID NO: 29. In some embodiments, the substitution is relative to a human germline light chain framework region sequence.

In some embodiments, the anti-TCRβV antibody molecule, e.g., anti-TCRβ V12 antibody molecule comprises a light chain variable domain comprising: (a) a framework region 1 (FR1) comprising a change, e.g., a substitution (e.g., a conservative substitution) at one or more (e.g., all) positions as described herein according to Kabat numbering, and (b) a framework region 3 (FR3) comprising a change, e.g., a substitution (e.g., a conservative substitution) at one or more (e.g., all) position as described herein according to Kabat numbering. In some embodiments, the substitution is relative to a human germline light chain framework region sequence.

In some embodiments, the anti-TCRβV antibody molecule, e.g., anti-TCRβ V12 antibody molecule comprises the heavy chain framework region 1. In some embodiments, the anti-TCRβV antibody molecule, e.g., anti-TCRβ V12 antibody molecule comprises the heavy chain framework region 2. In some embodiments, the anti-TCRβV antibody molecule, e.g., anti-TCRβ V12 antibody molecule comprises the heavy chain framework region 3. In some embodiments, the anti-TCRβV antibody molecule, e.g., anti-TCRβ V12 antibody molecule comprises the heavy chain framework region 4. In some embodiments, the anti-TCRβV antibody molecule, e.g., anti-TCRβ V12 antibody molecule comprises the heavy chain framework regions 1-4, e.g., SEQ ID NOS: 20-23. In some embodiments, the anti-TCRβV antibody molecule, e.g., anti-TCRβ V12 antibody molecule comprises the light chain framework regions 1-4, e.g., SEQ ID NOs: 26-30.

In some embodiments, the anti-TCRβV antibody molecule, e.g., anti-TCRβ V12 antibody molecule comprises the heavy chain framework regions 1-4, e.g., SEQ ID NOs: 23-25; and the light chain framework regions 1-4, e.g., SEQ ID NOs: 26-30.

In some embodiments, the heavy or light chain variable domain, or both, of, the anti-TCRβV antibody molecule, e.g., anti-TCRβ V12 antibody molecule includes an amino acid sequence, which is substantially identical to an amino acid as described herein, e.g., at least 80%, 85%, 90%, 92%, 95%, 97%, 98%, 99/o or higher identical to a variable region of an antibody described herein, e.g., an antibody as described in Table 2, or encoded by the nucleotide sequence in Table 2; or which differs at least 1 or 5 residues, but less than 40, 30, 20, or 10 residues, from a variable region of an antibody described herein.

In some embodiments, the anti-TCRβV antibody molecule, e.g., anti-TCRβ V12 antibody molecule comprises at least one, two, three, or four antigen-binding regions, e.g., variable regions, having an amino acid sequence as set forth in Table 2, or a sequence substantially identical thereto (e.g., a sequence at least about 85%, 90%, 95%, 99% or more identical thereto, or which differs by no more than 1, 2, 5, 10, or 15 amino acid residues from the sequences shown in Table 2. In another embodiment, the anti-TCRβV antibody molecule, e.g., anti-TCRβ V12 antibody molecule includes a VH and/or VL domain encoded by a nucleic acid having a nucleotide sequence as set forth in Table 2, or a sequence substantially identical thereto (e.g., a sequence at least about 85%, 90%, 95%, 99% or more identical thereto, or which differs by no more than 3, 6, 15, 30, or 45 nucleotides from the sequences shown in Table 2.

In some embodiments, the anti-TCRβV antibody molecule, e.g., anti-TCRβ V12 antibody molecule comprises: a VH domain comprising an amino acid sequence chosen from the amino acid sequence of SEQ ID NO: 23, SEQ ID NO:24 or SEQ ID NO:25, an amino acid sequence at least about 85%, 90%, 95%, 99% or more identical to the amino acid sequence SEQ ID NO: 23, SEQ ID NO:24 or SEQ ID NO:25, or an amino acid sequence which differs by no more than 1, 2, 5, 10, or 15 amino acid residues from the amino acid sequence of SEQ ID NO: 23, SEQ ID NO:24 or SEQ ID NO:25; and/or a VL domain comprising an amino acid sequence chosen from the amino acid sequence of SEQ ID NO: 26, SEQ ID NO: 27, SEQ ID NO: 28, SEQ ID NO: 29 or SEQ ID NO: 30, an amino acid sequence at least about 85%, 90%, 95%, 99% or more identical to the amino acid sequence of SEQ ID NO: 26, SEQ ID NO: 27, SEQ ID NO: 28, SEQ ID NO: 29 or SEQ ID NO: 30, or an amino acid sequence which differs by no more than 1, 2, 5, 10, or 15 amino acid residues from the amino acid sequence of SEQ ID NO: 26, SEQ ID NO: 27, SEQ ID NO: 28, SEQ ID NO: 29 or SEQ ID NO: 30. In some embodiments, the anti-TCRβV antibody molecule, e.g., anti-TCRβ V12 antibody molecule comprises: a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and a VL domain comprising the amino acid sequence of SEQ ID NO: 26, an amino acid sequence at least about 85%, 90%, 95%, 99% or more identical to the amino acid sequence SEQ ID NO: 26, or an amino acid sequence which differs by no more than 1, 2, 5, 10, or 15 amino acid residues from the amino acid sequence of SEQ ID NO: 26.

In some embodiments, the anti-TCRβV antibody molecule, e.g., anti-TCRβ V12 antibody molecule comprises: a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and a VL domain comprising the amino acid sequence of SEQ ID NO: 27, an amino acid sequence at least about 85%, 90%, 95%, 99% or more identical to the amino acid sequence SEQ ID NO: 27, or an amino acid sequence which differs by no more than 1, 2, 5, 10, or 15 amino acid residues from the amino acid sequence of SEQ ID NO: 27.

In some embodiments, the anti-TCRβV antibody molecule, e.g., anti-TCRβ V12 antibody molecule comprises: a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and a VL domain comprising the amino acid sequence of SEQ ID NO: 28, an amino acid sequence at least about 85%, 90%, 95%, 99% or more identical to the amino acid sequence SEQ ID NO: 28, or an amino acid sequence which differs by no more than 1, 2, 5, 10, or 15 amino acid residues from the amino acid sequence of SEQ ID NO: 28.

In some embodiments, the anti-TCRβV antibody molecule, e.g., anti-TCRβ V12 antibody molecule comprises: a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and a VL domain comprising the amino acid sequence of SEQ ID NO: 29, an amino acid sequence at least about 85%, 90%, 95%, 99% or more identical to the amino acid sequence SEQ ID NO: 29, or an amino acid sequence which differs by no more than 1, 2, 5, 10, or 15 amino acid residues from the amino acid sequence of SEQ ID NO: 29.

In some embodiments, the anti-TCRβV antibody molecule, e.g., anti-TCRβ V12 antibody molecule comprises: a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and a VL domain comprising the amino acid sequence of SEQ ID NO: 30, an amino acid sequence at least about 85%, 90%, 95%, 99% or more identical to the amino acid sequence SEQ ID NO: 30, or an amino acid sequence which differs by no more than 1, 2, 5, 10, or 15 amino acid residues from the amino acid sequence of SEQ ID NO: 30.

In some embodiments, the anti-TCRβV antibody molecule, e.g., anti-TCRβ V12 antibody molecule comprises: a VH domain comprising the amino acid sequence of SEQ ID NO: 24 or 25, an amino acid sequence at least about 85%, 90%, 95%, 99% or more identical to the amino acid sequence SEQ ID NO: 24 or 25, or an amino acid sequence which differs by no more than 1, 2, 5, 10, or 15 amino acid residues from the amino acid sequence of SEQ ID NO: 24 or 25; and a VL domain comprising the amino acid sequence of SEQ ID NO: 26, an amino acid sequence at least about 85%, 90%, 95%, 99/o or more identical to the amino acid sequence SEQ ID NO: 26, or an amino acid sequence which differs by no more than 1, 2, 5, 10, or 15 amino acid residues from the amino acid sequence of SEQ ID NO: 26.

In some embodiments, the anti-TCRβV antibody molecule, e.g., anti-TCRβ V12 antibody molecule comprises: a VH domain comprising the amino acid sequence of SEQ ID NO: 24 or 25, an amino acid sequence at least about 85%, 90%, 95%, 99% or more identical to the amino acid sequence SEQ ID NO: 24 or 25, or an amino acid sequence which differs by no more than 1, 2, 5, 10, or 15 amino acid residues from the amino acid sequence of SEQ ID NO: 24 or 25; and a VL domain comprising the amino acid sequence of SEQ ID NO: 27, an amino acid sequence at least about 85%, 90%, 95%, 99% or more identical to the amino acid sequence SEQ ID NO: 27, or an amino acid sequence which differs by no more than 1, 2, 5, 10, or 15 amino acid residues from the amino acid sequence of SEQ ID NO: 27.

In some embodiments, the anti-TCRβV antibody molecule, e.g., anti-TCRβ V12 antibody molecule comprises: a VH domain comprising the amino acid sequence of SEQ ID NO: 24 or 25, an amino acid sequence at least about 85%, 90%, 95%, 99% or more identical to the amino acid sequence SEQ ID NO: 24 or 25, or an amino acid sequence which differs by no more than 1, 2, 5, 10, or 15 amino acid residues from the amino acid sequence of SEQ ID NO: 24 or 25; and a VL domain comprising the amino acid sequence of SEQ ID NO: 28, an amino acid sequence at least about 85%, 90%, 95%, 99% or more identical to the amino acid sequence SEQ ID NO: 28, or an amino acid sequence which differs by no more than 1, 2, 5, 10, or 15 amino acid residues from the amino acid sequence of SEQ ID NO: 28.

In some embodiments, the anti-TCRβV antibody molecule, e.g., anti-TCRβ V12 antibody molecule comprises: a VH domain comprising the amino acid sequence of SEQ ID NO: 24 or 25, an amino acid sequence at least about 85%, 90%, 95%, 99% or more identical to the amino acid sequence SEQ ID NO: 24 or 25, or an amino acid sequence which differs by no more than 1, 2, 5, 10, or 15 amino acid residues from the amino acid sequence of SEQ ID NO: 24 or 25; and a VL domain comprising the amino acid sequence of SEQ ID NO: 29, an amino acid sequence at least about 85%, 90%, 95%, 99% or more identical to the amino acid sequence SEQ ID NO: 29, or an amino acid sequence which differs by no more than 1, 2, 5, 10, or 15 amino acid residues from the amino acid sequence of SEQ ID NO: 29.

In some embodiments, the anti-TCRβV antibody molecule, e.g., anti-TCRβ V12 antibody molecule comprises: a VH domain comprising the amino acid sequence of SEQ ID NO: 24 or 25, an amino acid sequence at least about 85%, 90%, 95%, 99% or more identical to the amino acid sequence SEQ ID NO: 24 or 25, or an amino acid sequence which differs by no more than 1, 2, 5, 10, or 15 amino acid residues from the amino acid sequence of SEQ ID NO: 24 or 25; and a VL domain comprising the amino acid sequence of SEQ ID NO: 30, an amino acid sequence at least about 85%, 90%, 95%, 99% or more identical to the amino acid sequence SEQ ID NO: 30, or an amino acid sequence which differs by no more than 1, 2, 5, 10, or 15 amino acid residues from the amino acid sequence of SEQ ID NO: 30.

In some embodiments, the anti-TCRβV antibody molecule, e.g., anti-TCRβ V12 antibody molecule comprises: a VH domain comprising the amino acid sequence of SEQ ID NO: 25 or 23, an amino acid sequence at least about 85%, 90%, 95%, 99% or more identical to the amino acid sequence SEQ ID NO: 25 or 23, or an amino acid sequence which differs by no more than 1, 2, 5, 10, or 15 amino acid residues from the amino acid sequence of SEQ ID NO: 25 or 23; and a VL domain comprising the amino acid sequence of SEQ ID NO: 26, an amino acid sequence at least about 85%, 90%, 95%, 99% or more identical to the amino acid sequence SEQ ID NO: 26, or an amino acid sequence which differs by no more than 1, 2, 5, 10, or 15 amino acid residues from the amino acid sequence of SEQ ID NO: 26.

In some embodiments, the anti-TCRβV antibody molecule, e.g., anti-TCRβ V12 antibody molecule comprises: a VH domain comprising the amino acid sequence of SEQ ID NO: 25 or 23, an amino acid sequence at least about 85%, 90%, 95%, 99% or more identical to the amino acid sequence SEQ ID NO: 25 or 23, or an amino acid sequence which differs by no more than 1, 2, 5, 10, or 15 amino acid residues from the amino acid sequence of SEQ ID NO: 25 or 23; and a VL domain comprising the amino acid sequence of SEQ ID NO: 27, an amino acid sequence at least about 85%, 90%, 95%, 99% or more identical to the amino acid sequence SEQ ID NO: 27, or an amino acid sequence which differs by no more than 1, 2, 5, 10, or 15 amino acid residues from the amino acid sequence of SEQ ID NO: 27.

In some embodiments, the anti-TCRβV antibody molecule, e.g., anti-TCRβ V12 antibody molecule comprises: a VH domain comprising the amino acid sequence of SEQ ID NO: 25 or 23, an amino acid sequence at least about 85%, 90%, 95%, 99% or more identical to the amino acid sequence SEQ ID NO: 25 or 23, or an amino acid sequence which differs by no more than 1, 2, 5, 10, or 15 amino acid residues from the amino acid sequence of SEQ ID NO: 25 or 23; and a VL domain comprising the amino acid sequence of SEQ ID NO: 28, an amino acid sequence at least about 85%, 90%, 95%, 99% or more identical to the amino acid sequence SEQ ID NO: 28, or an amino acid sequence which differs by no more than 1, 2, 5, 10, or 15 amino acid residues from the amino acid sequence of SEQ ID NO: 28.

In some embodiments, the anti-TCRβV antibody molecule, e.g., anti-TCRβ V12 antibody molecule comprises: a VH domain comprising the amino acid sequence of SEQ ID NO: 25 or 23, an amino acid sequence at least about 85%, 90%, 95%, 99% or more identical to the amino acid sequence SEQ ID NO: 25 or 23, or an amino acid sequence which differs by no more than 1, 2, 5, 10, or 15 amino acid residues from the amino acid sequence of SEQ ID NO: 25 or 23; and a VL domain comprising the amino acid sequence of SEQ ID NO: 29, an amino acid sequence at least about 85%, 90%, 95%, 99% or more identical to the amino acid sequence SEQ ID NO: 29, or an amino acid sequence which differs by no more than 1, 2, 5, 10, or 15 amino acid residues from the amino acid sequence of SEQ ID NO: 29.

In some embodiments, the anti-TCRβV antibody molecule, e.g., anti-TCRβ V12 antibody molecule comprises: a VH domain comprising the amino acid sequence of SEQ ID NO: 25 or 23, an amino acid sequence at least about 85%, 90%, 95%, 99% or more identical to the amino acid sequence SEQ ID NO: 25 or 23, or an amino acid sequence which differs by no more than 1, 2, 5, 10, or 15 amino acid residues from the amino acid sequence of SEQ ID NO: 25 or 23; and a VL domain comprising the amino acid sequence of SEQ ID NO: 30, an amino acid sequence at least about 85%, 90%, 95%, 99/o or more identical to the amino acid sequence SEQ ID NO: 30, or an amino acid sequence which differs by no more than 1, 2, 5, 10, or 15 amino acid residues from the amino acid sequence of SEQ ID NO: 30.

In some embodiments, the anti-TCRβV antibody molecule, e.g., anti-TCRβ V12 antibody molecule is a full antibody or fragment thereof (e.g., a Fab, F(ab′)2, Fv, or a single chain Fv fragment (scFv)). In embodiments, the anti-TCRβV antibody molecule, e.g., anti-TCRβ V6 (e.g., anti-TCRβ V6-5*01) antibody molecule is a monoclonal antibody or an antibody with single specificity. In some embodiments, the anti-TCRβV antibody molecule, e.g., anti-TCRβ V12 antibody molecule, can also be a humanized, chimeric, camelid, shark, or an in vitro-generated antibody molecule. In some embodiments, the anti-TCRβV antibody molecule, e.g., anti-TCRβ V12 antibody molecule is a humanized antibody molecule. The heavy and light chains of the anti-TCRβV antibody molecule, e.g., anti-TCRβ V12 antibody molecule can be full-length (e.g., an antibody can include at least one, and preferably two, complete heavy chains, and at least one, and preferably two, complete light chains) or can include an antigen-binding fragment (e.g., a Fab, F(ab′)2, Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRβV antibody molecule, e.g., anti-TCRβ V12 antibody molecule is in the form of a multispecific molecule, e.g., a bispecific molecule, e.g., as described herein.

In some embodiments, the anti-TCRβV antibody molecule, e.g., anti-TCRβ V12 antibody molecule has a heavy chain constant region (Fc) chosen from, e.g., the heavy chain constant regions of IgG1, IgG2, IgG3, IgG4, IgM, IgA1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgG1 or IgG2 (e.g., human IgG1, or IgG2). In some embodiments, the heavy chain constant region is human IgG1.

In some embodiments, the anti-TCRβV antibody molecule, e.g., anti-TCRβ V12 antibody molecule has a light chain constant region chosen from, e.g., the light chain constant regions of kappa or lambda, preferably kappa (e.g., human kappa). In some embodiments, the constant region is altered, e.g., mutated, to modify the properties of the anti-TCRβV antibody molecule, e.g., anti-TCRβ V12 antibody molecule (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 212 or 214; or positions 135 (M to Y), 137 (S to T), 139 (T to E), 316 (H to K) and 317 (N to F) of SEQ ID NOs: 215, 216, 217 or 218).

Antibody B-H.1 comprises a first chain comprising the amino acid sequence of SEQ ID NO: 3280 and a second chain comprising the amino acid sequence of SEQ ID NO: 3281.

Additional exemplary anti-TCRβ V12 antibodies are provided in Table 2. In some embodiments, the anti-TCRβ V12 is antibody B, e.g., humanized antibody B (antibody B-H), as provided in Table 2. In some embodiments, the anti-TCRβV antibody comprises one or more (e.g., all three) of a LC CDR1, LC CDR2, and LC CDR3 provided in Table 2, and/or one or more (e.g., all three) of a HC CDR1, HC CDR2, and HC CDR3 provided in Table 2, or a sequence with at least 95% sequence identity thereto. In some embodiments, antibody B comprises a variable heavy chain (VH) and/or a variable light chain (VL) provided in Table 2, or a sequence with at least 95% sequence identity thereto.

In some embodiments, the anti-TCRVB 12 antibody molecule (e.g., anti-TCRVB 12-3 or anti-TCRVB 12-4 antibody molecule) comprises a VH of B-H.1A, B-H.1B, B-H.1C, B-H.1D, B-H.1E, B-H.1F, B-H.1G, B-H.1H, B-H.1, B-H.2, B-H.3, B-H.4, B-H.5, or B-H.6, or a sequence with at least 80%, 85%, 90%, 95%, 96%, 97%, 98%, 99% or more identity thereto.

In some embodiments, the anti-TCRVB 12 antibody molecule (e.g., anti-TCRVB 12-3 or anti-TCRVB 12-4 antibody molecule) comprises a VL of B-H.1A, B-H.1B, B-H.1C, B-H.1D, B-H.1E, B-H.1F, B-H.1G, B-H.1H, B-H.1, B-H.2, B-H.3, B-H.4, B-H.5, or B-H.6, or a sequence with at least 80%, 85%, 90%, 95%, 96%, 97%, 98%, 99% or more identity thereto.

In some embodiments, the anti-TCRVB 12 antibody molecule (e.g., anti-TCRVB 12-3 or anti-TCRVB 12-4 antibody molecule) comprises a VH of B-H.1A, B-H.1B, B-H.1C, B-H.1D, B-H.1E, B-H.1F, B-H.1G, B-H.1H, B-H.1, B-H.2, B-H.3, B-H.4, B-H.5, or B-H.6, or a sequence with at least 80%, 85%, 90%, 95%, 96%, 97%, 98%, 99% or more identity thereto; and a VL of B-H.1A, B-H.1B, B-H.1C, B-H.1D, B-H.1E, B-H.1F, B-H.1G, B-H.1H, B-H.1, B-H.2, B-H.3, B-H.4, B-H.5, or B-H.6, or a sequence with at least 80%, 85%, 90%, 95%, 96%, 97%, 98%, 99% or more identity thereto.

### Anti-TCRβ V10 Antibodies

In one aspect, provided herein is an anti-TCRβV antibody molecule that binds to a human TCRβ V10 subfamily member. In some embodiments, TCRβ V10 subfamily is also known as TCR Vβ12. In some embodiments, the TCRβ V10 subfamily comprises: TCRβ V10-1*01, TCRβ V10-1*02, TCRβ V10-3*01 or TCRβ V10-2*01, or a variant thereof.

Exemplary anti-TCRβ V10 antibodies are provided in Table 12. In some embodiments, the anti-TCRβ V10 is antibody D. e.g., humanized antibody D (antibody D-H), as provided in Table 12. In some embodiments, antibody D comprises one or more (e.g., three) light chain CDRs and/or one or more (e.g., three) heavy chain CDRs provided in Table 12, or a sequence with at least 95% sequence identity thereto. In some embodiments, antibody D comprises a variable heavy chain (VH) and/or a variable light chain (VL) provided in Table 12, or a sequence with at least 95% sequence identity thereto.

In some embodiments, the anti-TCRβ V10 antibody molecule comprises a VH and/or a VL of an antibody described in Table 12, or a sequence with at least 80%, 85%, 90%, 95%, 96%, 97%, 98%, 99% or more identity thereto.

In some embodiments, the anti-TCRβ V10 antibody molecule comprises a VH and a VL of an antibody described in Table 12, or a sequence with at least 80%, 85%, 90%, 95%, 96%, 97%, 98%, 99% or more identity thereto.

### Anti-TCRβ V19 Antibodies

In one aspect, provided herein is an anti-TCRβV antibody molecule that binds to a human TCRβ V19 subfamily member. In some embodiments, the TCRβ V19 subfamily comprises: TCRβ V19-1*01, TCRβ V19-1*02, or TCRβ V19-1*03, or a variant thereof.

Exemplary anti-TCRβ V19 antibodies are provided in Table 18. In some embodiments, the anti-TCRβ V19 is antibody Q, as provided in Table 18. In some embodiments, antibody Q comprises one or more (e.g., three) light chain CDRs and/or one or more (e.g., three) heavy chain CDRs provided in Table 18, or a sequence with at least 95% sequence identity thereto. In some embodiments, antibody Q comprises a variable heavy chain (VH) and/or a variable light chain (VL) provided in Table 18, or a sequence with at least 95% sequence identity thereto.

In some embodiments, the anti-TCRβ V19 antibody molecule comprises a VH and/or a VL of an antibody described in Table 18, or a sequence with at least 80%, 85%, 90%, 95%, 96%, 97%, 98%, 99% or more identity thereto.

In some embodiments, the anti-TCRβ V19 antibody molecule comprises a VH and a VL of an antibody described in Table 18, or a sequence with at least 80%, 85%, 90%, 95%, 96%, 97%, 98%, 99% or more identity thereto.

### Anti-TCRβ V20 Antibodies

In one aspect, provided herein is an anti-TCRβV antibody molecule that binds to a human TCRβ V20 subfamily member. In some embodiments, the TCRβ V20 subfamily comprises: TCRβ V20-1*01, TCRβ V20-1*02, TCRβ V20-1*03, TCRβ V20-1*04, TCRβ V20-1*05, TCRβ V20-1*06, or TCRβ V20-1*07, or a variant thereof.

Exemplary anti-TCRβ V20 antibodies are provided in Table 15. In some embodiments, the anti-TCRβ V20 is antibody L, as provided in Table 15. In some embodiments, antibody L comprises one or more (e.g., three) light chain CDRs and/or one or more (e.g., three) heavy chain CDRs provided in Table 15, or a sequence with at least 95% sequence identity thereto. In some embodiments, antibody L comprises a variable heavy chain (VH) and/or a variable light chain (VL) provided in Table 15, or a sequence with at least 95% sequence identity thereto.

In some embodiments, the anti-TCRβ V20 is antibody M, as provided in Table 15. In some embodiments, antibody M comprises one or more (e.g., three) light chain CDRs and/or one or more (e.g., three) heavy chain CDRs provided in Table 15, or a sequence with at least 95% sequence identity thereto.

In some embodiments, antibody M comprises a variable heavy chain (VH) and/or a variable light chain (VL) provided in Table 15, or a sequence with at least 95% sequence identity thereto.

In some embodiments, the anti-TCRβ V20 antibody molecule comprises a VH and/or a VL of an antibody described in Table 15, or a sequence with at least 80%, 85%, 90%, 95%, 96%, 97%, 98%, 99% or more identity thereto.

In some embodiments, the anti-TCRβ V20 antibody molecule comprises a VH and a VL of an antibody described in Table 15, or a sequence with at least 80%, 85%, 90%, 95%, 96%, 97%, 98%, 99% or more identity thereto.

### Anti-TCRβ V28 Antibodies

In one aspect, provided herein is an anti-TCRβV antibody molecule that binds to a human TCRβ V28 subfamily member. In some embodiments, the TCRβ V28 subfamily comprises: TCRβ V28-1*01, or a variant thereof.

Exemplary anti-TCRβ V28 antibodies are provided in Table 17. In some embodiments, the anti-TCRβ V28 is antibody P, as provided in Table 17. In some embodiments, antibody P comprises one or more (e.g., three) light chain CDRs and/or one or more (e.g., three) heavy chain CDRs provided in Table 17, or a sequence with at least 95% sequence identity thereto. In some embodiments, antibody P comprises a variable heavy chain (VH) and/or a variable light chain (VL) provided in Table 17, or a sequence with at least 95% sequence identity thereto.

In some embodiments, the anti-TCRβ V28 antibody molecule comprises a VH and/or a VL of an antibody described in Table 17, or a sequence with at least 80%, 85%, 90%, 95%, 96%, 97%, 98%, 99% or more identity thereto.

In some embodiments, the anti-TCRβ V28 antibody molecule comprises a VH and a VL of an antibody described in Table 17, or a sequence with at least 80%, 85%, 90%, 95%, 96%, 97%, 98%, 99% or more identity thereto.

### Anti-TCRβV15 Antibodies

In one aspect, provided herein is an anti-TCRβV antibody molecule that binds to a human TCRβ V15 subfamily member. In some embodiments, the TCRβ V15 subfamily comprises: TCRβ V15*01, TCRβ V15*02, or TCRβ V15*03, or a variant thereof.

### Anti-TCRβ V7 Antibodies

In one aspect, provided herein is an anti-TCRβV antibody molecule that binds to a human TCRβ V7 subfamily member. In some embodiments, the TCRβ V7 subfamily comprises: TCRβ V7-1*01, TCRβ V7-2*01, TCRβ V7-2*02, TCRβ V2-1*03, TCRβ V7-2*04, TCRβ V7-3*01, TCRβ V7-3*02, TCRβ V7-3*03, TCRβ V7-3*04, TCRβ V7-3*05, TCRβ V7-4*01, TCRβ V7-4*02, TCRβ V7-5*01, TCRβ V7-5*02, TCRβ V7-6*01, TCRβ V7-6*02, TCRβ V7-7*01, TCRβ V7-7*02, TCRβ V7-8*01, TCRβ V7-8*02, TCRβ V7-8*03, TCRβ V7-9*01, TCRβ V7-9*02, TCRβ V7-9*03, TCRβ V7-9*04, TCRβ V7-9*05, TCRβ V7-9*06, or TCRβ V7-9*07, or a variant thereof.

### Murine Anti-TCRβ V17 Antibodies

In one aspect, provided herein is an anti-TCRβV antibody molecule that binds to a murine TCRβ V17 subfamily member. In some embodiments, the murine TCRβ V17 subfamily comprises: TCRβ V17-1*01, or a variant thereof.

Exemplary anti-TCRβ V17 antibodies are provided in Table 18. In some embodiments, the anti-TCRβ V17 is antibody R, as provided in Table 19. In some embodiments, antibody R comprises one or more (e.g., three) light chain CDRs and/or one or more (e.g., three) heavy chain CDRs provided in Table 19, or a sequence with at least 95% sequence identity thereto. In some embodiments, antibody Q comprises a variable heavy chain (VH) and/or a variable light chain (VL) provided in Table 19, or a sequence with at least 95% sequence identity thereto.

In some embodiments, the anti-TCRβ V17 antibody molecule comprises a VH and/or a VL of an antibody described in Table 19, or a sequence with at least 80%, 85%, 90%, 95%, 96%, 97%, 98%, 99% or more identity thereto.

In some embodiments, the anti-TCRβ V17 antibody molecule comprises a VH and a VL of an antibody described in Table 19, or a sequence with at least 80%, 85%, 90%, 95%, 96%, 97%, 98%, 99% or more identity thereto.

### Murine Anti-TCRβ V29 Antibodies

In one aspect, provided herein is an anti-TCRβV antibody molecule that binds to a murine TCRβ V29 subfamily member. In some embodiments, the murine TCRβ V29 subfamily comprises: TCRβ V29-1*01, or TCRβ V29-1*02, or a variant thereof.

Exemplary anti-TCRβ V29 antibodies are provided in Table 20. In some embodiments, the anti-TCRβ V18 is antibody S, as provided in Table 20. In some embodiments, antibody S comprises one or more (e.g., three) light chain CDRs and/or one or more (e.g., three) heavy chain CDRs provided in Table 20, or a sequence with at least 95% sequence identity thereto. In some embodiments, antibody S comprises a variable heavy chain (VH) and/or a variable light chain (VL) provided in Table 20, or a sequence with at least 95% sequence identity thereto.

In some embodiments, the anti-TCRβ V29 antibody molecule comprises a VH and/or a VL of an antibody described in Table 20, or a sequence with at least 80%, 85%, 90%, 95%, 96%, 97%, 98%, 99% or more identity thereto.

In some embodiments, the anti-TCRβ V29 antibody molecule comprises a VH and a VL of an antibody described in Table 20, or a sequence with at least 80%, 85%, 90%, 95%, 96%, 97%, 98%, 99% or more identity thereto.

### Murine Anti-TCRβ V13 Antibodies

In one aspect, provided herein is an anti-TCRβV antibody molecule that binds to a murine TCRβ V13 subfamily member. In some embodiments, the murine TCRβ V13 subfamily comprises: TCRβ V13-1*01, TCRβ V13-1*02, TCRβ V13-2*01, TCRβ V13-2*02, TCRβ V13-2*03, TCRβ V13-2*04, TCRβ V13-2*05, or TCRβ V13-3*01, or a variant thereof.

Exemplary anti-TCRβ V13 antibodies are provided in Table 21. In some embodiments, the anti-TCRβ V13 is antibody T, as provided in Table 21. In some embodiments, antibody S comprises one or more (e.g., three) light chain CDRs and/or one or more (e.g., three) heavy chain CDRs provided in Table 21, or a sequence with at least 95% sequence identity thereto. In some embodiments, antibody T comprises a variable heavy chain (VH) and/or a variable light chain (VL) provided in Table 21, or a sequence with at least 95% sequence identity thereto.

In some embodiments, the anti-TCRβ V13 antibody molecule comprises a VH and/or a VL of an antibody described in Table 21, or a sequence with at least 80%, 85%, 90%, 95%, 96%, 97%, 98%, 99% or more identity thereto.

In some embodiments, the anti-TCRβ V13 antibody molecule comprises a VH and a VL of an antibody described in Table 21, or a sequence with at least 80%, 85%, 90%, 95%, 96%, 97%, 98%, 99% or more identity thereto.

Additional anti-TCRVβ Antibodies

Additional exemplary anti-TCRβV antibodies are provided in Table 13. In some embodiments, the anti-TCRβV antibody is a humanized antibody, e.g., as provided in Table 13. In some embodiments, the anti-TCRβV antibody comprises one or more (e.g., all three) of a LC CDR1, LC CDR2, and LC CDR3 provided in Table 13; and/or one or more (e.g., all three) of a HC CDR1, HC CDR2, and HC CDR3 provided in Table 13, or a sequence with at least 95% sequence identity thereto. In some embodiments, the anti-TCRβV antibody comprises a variable heavy chain (VH) and/or a variable light chain (VL) provided in Table 13, or a sequence with at least 95% sequence identity thereto.

### Anti-TCRαV Antibodies

### Human T Cell Receptor (TCR) Complex

TCR is a disulfide-linked membrane-anchored heterodimeric protein normally consisting of the highly variable alpha (α) and beta (β) chains expressed as part of a complex with the invariant CD3 chain molecules. TCR on αβ T cells is formed by a heterodimer of one alpha chain and one beta chain. Each alpha or beta chain consists of a constant domain and a highly variable domain classified as the Immunoglobulin superfamily (IgSF) fold. The TCRαV chains can be further classified into subfamilies (TRAV12, 13, 19, 21, or 30). Despite their high structural and functional homology, the amino acid sequence homology in the TRAV genes is very low. Nevertheless, TCRs formed between alpha and beta chains of highly diverse sequences show a remarkable structural and elicit a similar function, e.g., activation of T cells.

T cell receptors (TCR) can be found on the surface of T cells. TCRs recognize antigens, e.g., peptides, presented on, e.g., bound to, major histocompatibility complex (MHC) molecules on the surface of cells, e.g., antigen-presenting cells. TCRs are heterodimeric molecules and can comprise an alpha chain, a beta chain, a gamma chain or a delta chain. TCRs comprising an alpha chain and a beta chain are also referred to as TCRαβ. The TCR beta chain consists of the following regions (also known as segments); variable (V), diversity (D), joining (J) and constant (C) (see Mayer G. and Nyland J. (2010) Chapter 10: Major Histocompatibility Complex and T-cell Receptors-Role in Immune Responses. In: Microbiology and Immunology on-line, University of South Carolina School of Medicine). The TCR alpha chain consists of V. J and C regions. The rearrangement of the T-cell receptor (TCR) through somatic recombination of V (variable), D (diversity), J (joining), and C (constant) regions is a defining event in the development and maturation of a T cell. TCR gene rearrangement takes place in the thymus.

TCRs can comprise a receptor complex, known as the TCR complex, which comprises a TCR heterodimer comprising of an alpha chain and a beta chain, and dimeric signaling molecules, e.g., CD3 co-receptors, e.g., CD3δ/ε, and/or CD3γ/ε.

As used herein, the term “T cell receptor alpha variable chain” or “TCRαV,” or “TRAV,” refers to an extracellular region of the T cell receptor alpha chain which can comprise a portion of the antigen recognition domain of the T cell receptor. The term TCRαV includes isoforms, mammalian, e.g., human TCRαV, species homologs of human and analogs comprising at least one common epitope with TCRαV. Human TCRαV comprises a gene family comprising subfamilies including, but not limited to: a TCRα V12 subfamily, a TCRα V13 subfamily, a TCRα V19 subfamily, a TCRα V21 subfamily, or a TCRα V30 subfamily, as well as family members of said subfamilies, and variants thereof (e.g., a structural or functional variant thereof).

In some embodiments, the TCRα V12 subfamily comprises: TCRαV12-1, TCRαV12-2, or TCRαV12-3, or a variant thereof.

In some embodiments, the TCRα V13 subfamily comprises: TCRαV13-1 or TCRαV13-2, or a variant thereof.

### TCR Alpha V (TCRαV)

Diversity in the immune system enables protection against a huge array of pathogens. Since the germline genome is limited in size, diversity is achieved not only by the process of V(D)J recombination but also by junctional (junctions between V-D and D-J segments) deletion of nucleotides and addition of pseudo-random, non-templated nucleotides. The TCR alpha gene undergoes gene arrangement to generate diversity.

The TCR V alpha repertoire varies between individuals and populations because of, e.g., 7 frequently occurring inactivating polymorphisms in functional gene segments and a large insertion/deletion-related polymorphism encompassing 2 V alpha gene segments.

Provided herein are, inter alia, antibody molecules and fragments thereof, that bind, e.g., specifically bind, to a human TCR alpha V chain (TCRαV), e.g., a TCRαV gene family (also referred to as a group), e.g., a TCRαV subfamily (also referred to as a subgroup), e.g., as described herein. TCR alpha V families and subfamilies are known in the art, e.g., as described in Yassai et al., (2009) Immunogenetics 61(7) pp: 493-502; Wei S. and Concannon P. (1994) Human Immunology 41(3) pp: 201-206. The antibodies described herein can be recombinant antibodies, e.g., recombinant non-murine antibodies, e.g., recombinant human or humanized antibodies.

The terms TCRAV, TCRVA, TRAV, TCRαV, TCRVα or TRαV are used interchangeably herein and refer to a TCR alpha V chain, e.g., as described herein.

In some embodiments, provided herein is an anti-TCRαV antibody molecule that binds to human TCRαV, e.g., a TCRαV family, e.g., gene family or a variant thereof.

Exemplary amino acid sequences for TCRαV subfamily members can be found on the ImMunoGeneTics Information System website: http://www.imgt.org/, or in a similar resource.

### Anti-TCRαV Antibodies

Current bispecific constructs designed to redirect T cells to promote tumor cell lysis for cancer immunotherapy typically utilize antibody fragments (Fab, scFv, VH, single domain antibody, etc.) that are derived from monoclonal antibodies (mAb) directed against the CD3e subunit of the T cell receptor (TCR). However, there are limitations to this approach which may prevent the full realization of the therapeutic potential for such bispecific constructs. Previous studies have shown that even low “activating” doses of anti-CD3e mAb can cause long-term T cell dysfunction and exert immunosuppressive effects. In addition, anti-CD3e mAbs have been associated with side effects that result from massive T cell activation. The large number of activated T cells secrete substantial amounts of cytokines, the most important of which is Interferon gamma (IFNγ). This excess amount of IFNγ in turn activates macrophages which then overproduce proinflammatory cytokines such as IL-1beta, IL-6, IL-10 and TNF-alpha, causing a “cytokine storm” known as the cytokine release syndrome (CRS) (Shimabukuro-Vornhagen et al., J Immunother Cancer. 2018 Jun. 15; 6(1):56, herein incorporated by reference in its entirety). Thus, the need exists for developing antibodies that are capable of binding and activating only a subset of effector T cells, e.g., to re-duce the CRS and/or neurotoxicity (NT).

Described herein are molecules targeting the TCRαV chain of TCR and methods thereof. Without wishing to be bound by theory, such molecules are capable of binding, activating, and/or expanding only a subset of T cells, avoiding or reducing CRS and/or NT and minimizing potential immunosuppressive effects of anti-CD3 mAbs.

Described herein is a class of antibodies, i.e., anti-TCRαV antibody molecules as described herein, which despite having low sequence similarity (e.g., low sequence identity among the different antibody molecules that recognize different TCRαV subfamilies), recognize a structurally conserved, yet sequence-wise variable, region, e.g., domain, on the TCRαV protein and have a similar function (e.g., activation of T cells and a similar cytokine profile as described herein). Thus, the anti-TCRαV antibody molecules as described herein share a structure-function relationship.

In some embodiments, the anti-TCRαV antibody molecules as described herein do not recognize, e.g., bind to, an interface of a TCRβV:TCRalpha complex. In some embodiments, the anti-TCRαV antibody molecules as described herein do not recognize, e.g., bind to, a constant region of a TCRβV protein. In some embodiments, the anti-TCRαV antibody molecules as described herein do not recognize, e.g., bind to, one or more (e.g., all) of a complementarity determining region (e.g., CDR1, CDR2 and/or CDR3) of a TCRβV protein.

Provided herein are, inter alia, antibody molecules directed to the variable chain of the alpha subunit of TCR (TCRαV) which bind and, e.g., activate a subset of T cells. The anti-TCRαV antibody molecules as described herein result in lesser or no production of cytokines associated with CRS, e.g., IL-6, IL-1beta, IL-10 and TNF alpha; and enhanced and/or delayed production of IL-2 and IFNγ. In some embodiments, the anti-TCRαV antibodies as described herein have a cytokine profile, e.g., as described herein, which differs from a cytokine profile of a T cell engager that binds to a receptor or molecule other than a TCRαV region (“a non-TCRαV-binding T cell engager”). In some embodiments, the non-TCRαV-binding T cell engager comprises an antibody that binds to a CD3 molecule (e.g., CD3 epsilon (CD3e) molecule); or a TCR alpha (TCRα) molecule. In some embodiments, the non-TCRαV-binding T cell engager is an OKT3 antibody or an SP34-2 antibody.

In some embodiments, the anti-TCRαV antibodies as described herein result in expansion of TCRαV+ T cells, e.g., a subset of memory effector T cells known as TEMRA. Without wishing to be bound by theory, it is believed that in some embodiments, TEMRA cells can promote tumor cell lysis but not CRS. Accordingly, provided herein are methods of making said anti-TCRαV antibody molecules and uses thereof. Also described herein are multispecific molecules, e.g., bispecific molecules comprising said anti-TCRαV antibody molecules. In some embodiments, compositions comprising anti-TCRαV antibody molecules of the present disclosure, can be used, e.g., to: (1) activate and redirect T cells to promote tumor cell lysis for cancer immuno-therapy, and/or (2) expand TCRαV+ T cells. In some embodiments, compositions comprising anti-TCRαV antibody molecules as described herein limit the harmful side-effects of CRS and/or NT, e.g., CRS and/or NT associated with anti-CD3e targeting.

In some embodiments, the anti-TCRαV antibody molecule binds to one or more of TRAV12-1, TRAV12-2, TRAV12-3, TRAV13-1, TRAV13-2, TRAV19-1, TRAV21-1, and TRAV30-1. In some embodiments, the anti-TCRαV antibody molecule is an anti-TRAV12-1, anti-TRAV12-2, anti-TRAV12-3, anti-TRAV13-1, anti-TRAV13-2, anti-TRAV19-1, anti-TRAV21-1, or anti-TRAV30-1.

In some embodiments, the anti-TCRαV antibody molecule binds specifically to TRAV12-1, TRAV12-2, TRAV12-3, TRAV13-1, TRAV13-2, TRAV19-1, TRAV21-1, or TRAV30-1.

In some embodiments, the anti-TCRαV antibody molecule is a full antibody or fragment thereof (e.g., a Fab, F(ab′)2, Fv, single domain antibody, or a single chain Fv fragment (scFv)). In embodiments, the anti-TCRαV antibody molecule is a monoclonal antibody or an antibody with single specificity. In some embodiments, the anti-TCRαV antibody molecule can also be a humanized, chimeric, camelid, shark, or an in vitro-generated antibody molecule. In some embodiments, the anti-TCRαV antibody molecule is a humanized antibody molecule. The heavy and light chains of the anti-TCRαV antibody molecule can be full-length (e.g., an antibody can include at least one, and preferably two, complete heavy chains, and at least one, and preferably two, complete light chains) or can include an antigen-binding fragment (e.g., a Fab, F(ab′)2, Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRαV antibody molecule is in the form of a multispecific molecule, e.g., a bispecific molecule, e.g., as described herein.

In some embodiments, the anti-TCRαV antibody molecule has a heavy chain constant region (Fc) chosen from, e.g., the heavy chain constant regions of IgG1, IgG2, IgG3, IgG4, IgM, IgA1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgG1 or IgG2 (e.g., human IgG1, or IgG2). In some embodiments, the heavy chain constant region is human IgG1. In some embodiments, the Fc region comprises a Fc region variant, e.g., as described herein.

In some embodiments, the anti-TCRαV antibody molecule has a light chain constant region chosen from, e.g., the light chain constant regions of kappa or lambda, preferably kappa (e.g., human kappa). In some embodiments, the constant region is altered, e.g., mutated, to modify the properties of the anti-TCRαV antibody molecule (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 212 or 214; or positions 135 (M to Y), 137 (S to T), 139 (T to E), 316 (H to K) and 317 (N to F) of SEQ ID NOs: 215, 216, 217 or 218), e.g., relative to human IgG1.

The various TCRαV subfamilies and/or subfamily members can be expressed at different levels in individuals, e.g., healthy individuals, as disclosed in Kitaura K. et al (2016), BMC Immunology vol 17: 38, the entire contents of which are hereby incorporated by reference.

In some embodiments, the anti-TCRαV antibody molecule is a non-murine antibody molecule, e.g., a human or humanized antibody molecule. In some embodiments, the anti-TCRαV antibody molecule is a human antibody molecule. In some embodiments, the anti-TCRαV antibody molecule is a humanized antibody molecule.

In some embodiments, the anti-TCRαV antibody molecule is isolated or recombinant.

In some embodiments, the anti-TCRαV antibody molecule comprises a heavy chain constant region for an IgG4, e.g., a human IgG4. In still another embodiment, the anti-TCRαV antibody molecule includes a heavy chain constant region for an IgG1, e.g., a human IgG1. In some embodiments, the heavy chain constant region comprises an amino sequence set forth in Table 3, or a sequence substantially identical (e.g., at least 80%, 85%, 90%, 92%, 95%, 97%, 98%, 99% or higher identical) thereto.

In some embodiments, the anti-TCRαV antibody molecule includes a kappa light chain constant region, e.g., a human kappa light chain constant region. In some embodiments, the light chain constant region comprises an amino sequence set forth in Table 3, or a sequence substantially identical (e.g., at least 80%, 85%, 90%, 92%, 95%, 97%, 98%, 99% or higher identical) thereto.

In some embodiments, e.g., an embodiment comprising a variable region, a CDR (e.g., a combined CDR, Chothia CDR or Kabat CDR), or other sequence referred to herein, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments the antibody molecule comprises a multispecific molecule, e.g., a bispecific molecule, e.g., as described herein.

In some embodiments, the anti-TCRαV antibody molecule, is a non-murine antibody molecule, e.g., a human or humanized antibody molecule. In some embodiments, the anti-TCRαV antibody molecule is a human antibody molecule. In some embodiments, the anti-TCRαV antibody molecule is a humanized antibody molecule.

In some embodiments, the anti-TCRαV antibody molecule, is isolated or recombinant.

In some embodiments, the anti-TCRαV antibody molecule can contain any combination of CDRs or hypervariable loops according to the Kabat and Chothia definitions.

In some embodiments, e.g., an embodiment comprising a variable region, a CDR (e.g., a combined CDR, Chothia CDR or Kabat CDR), or other sequence referred to herein, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments the antibody molecule comprises a multispecific molecule, e.g., a bispecific molecule, e.g., as described herein.

In some embodiments, the anti-TCRαV antibody molecule comprises a light chain variable domain comprising: (a) a framework region 1 (FR1) comprising a change, e.g., a substitution (e.g., a conservative substitution) at one or more (e.g., all) positions as described herein according to Kabat numbering, and (b) a framework region 3 (FR3) comprising a change, e.g., a substitution (e.g., a conservative substitution) at one or more (e.g., all) position as described herein according to Kabat numbering. In some embodiments, the substitution is relative to a human germline light chain framework region sequence.

In some embodiments, the anti-TCRαV antibody molecule is a full antibody or fragment thereof (e.g., a Fab, F(ab′)2, Fv, or a single chain Fv fragment (scFv)). In embodiments, the anti-TCRαV antibody molecule is a monoclonal antibody or an antibody with single specificity. In some embodiments, the anti-TCRαV antibody molecule can also be a humanized, chimeric, camelid, shark, or an in vitro-generated antibody molecule. In some embodiments, the anti-TCRαV antibody molecule is a humanized antibody molecule. The heavy and light chains of the anti-TCRαV antibody molecule can be full-length (e.g., an antibody can include at least one, and preferably two, complete heavy chains, and at least one, and preferably two, complete light chains) or can include an antigen-binding fragment (e.g., a Fab, F(ab′)2, Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRαV antibody molecule is in the form of a multispecific molecule, e.g., a bispecific molecule, e.g., as described herein.

In some embodiments, the anti-TCRαV antibody molecule has a heavy chain constant region (Fc) chosen from, e.g., the heavy chain constant regions of IgG1, IgG2, IgG3, IgG4, IgM, IgA1. IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgG1 or IgG2 (e.g., human IgG1, or IgG2). In some embodiments, the heavy chain constant region is human IgG1.

In some embodiments, the anti-TCRαV antibody molecule has a light chain constant region chosen from, e.g., the light chain constant regions of kappa or lambda, preferably kappa (e.g., human kappa). In some embodiments, the constant region is altered, e.g., mutated, to modify the properties of the anti-TCRαV antibody molecule (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 212 or 214; or positions 135 (M to Y), 137 (S to T), 139 (T to E), 316 (H to K) and 317 (N to F) of SEQ ID NOs: 215, 216, 217 or 218).

### Anti-TCRα V12 Antibodies

In one aspect, provided herein is an anti-TCRαV antibody molecule that binds to a human TCRα V12 subfamily member. In some embodiments, the TCRα V12 subfamily comprises: TCRα V12-1*01, TCRα V12-1*02, TCRα V12-2*01, TCRα V12-2*02, TCRα V12-2*03, TCRα V12-3*01, or TCRα V12-3*02, or a variant thereof.

### Anti-TCRα V13 Antibodies

In one aspect, provided herein is an anti-TCRαV antibody molecule that binds to a human TCRα V13 subfamily member. In some embodiments, the TCRα V13 subfamily comprises: TCRα V13-1*01, TCRαV13-1*02, TCRαV13-1*03, TCRαV13-2*01, or TCRαV13-2*02, or a variant thereof.

### Anti-TCRα V19 Antibodies

In one aspect, provided herein is an anti-TCRαV antibody molecule that binds to a human TCRα V19 subfamily member. In some embodiments, the TCRα V19 subfamily comprises: TCRα V19-1*01, or a variant thereof.

Exemplary anti-TCRα V19 antibodies are provided in Table 22. In some embodiments, the anti-TCRα V19 is antibody U, as provided in Table 22. In some embodiments, antibody U comprises one or more (e.g., three) light chain CDRs and/or one or more (e.g., three) heavy chain CDRs provided in Table 22, or a sequence with at least 95% sequence identity thereto. In some embodiments, antibody T comprises a variable heavy chain (VH) and/or a variable light chain (VL) provided in Table 22, or a sequence with at least 95% sequence identity thereto.

In some embodiments, the anti-TCRα V19 antibody molecule comprises a VH and/or a VL of an antibody described in Table 22, or a sequence with at least 80%, 85%, 90%, 95%, 96%, 97%, 98%, 99% or more identity thereto.

In some embodiments, the anti-TCRβ V19 antibody molecule comprises a VH and a VL of an antibody described in Table 22, or a sequence with at least 80%, 85%, 90%, 95%, 96%, 97%, 98%, 99% or more identity thereto.

### Anti-TCRα V21 Antibodies

In one aspect, provided herein is an anti-TCRαV antibody molecule that binds to a human TCRα V21 subfamily member. In some embodiments, TCRα V21 subfamily is also known as TCRα V21. In some embodiments, the TCRα V21 subfamily comprises: TCRα V21-1*01, or TCRα V21-1*02, or a variant thereof.

### Anti-TCRα V30 Antibodies

In one aspect, provided herein is an anti-TCRαV antibody molecule that binds to a human TCRα V30 subfamily member. In some embodiments, TCRα V30 subfamily is also known as TCRα V30. In some embodiments, the TCRα V30 subfamily comprises: TCRα V30-1*01, TCRα V30-1*02, TCRα V30-1*03, TCRα V30-1*04, or TCRα V30-1*05, or a variant thereof.

### Antibody-Like Frameworks or Scaffolds

A wide variety of antibody/immunoglobulin frameworks or scaffolds can be employed in the anti-TCRvb or anti-TCRva antibody molecules as described herein or multifunctional formats thereof so long as the resulting polypeptide includes at least one binding region which specifically binds to the target antigen, e.g., a TCRvb or TCRva, a tumor antigen, among others. Such frameworks or scaffolds include the 5 main idiotypes of human immunoglobulins, or fragments thereof, and include immunoglobulins of other animal species, preferably having humanized aspects. Novel frameworks, scaffolds and fragments continue to be discovered and developed by those skilled in the art.

In some embodiments, the anti-TCRvb or anti-TCRva antibody molecules as described herein or multifunctional formats thereof include non-immunoglobulin based antibodies using non-immunoglobulin scaffolds onto which CDRs can be grafted. Any non-immunoglobulin frameworks and scaffolds may be employed, as long as they comprise a binding region specific for the target antigen (e.g., TCRvb, TCRva, or a tumor antigen). Exemplary non-immunoglobulin frameworks or scaffolds include, but are not limited to, fibronectin (Compound Therapeutics, Inc., Waltham, MA), ankyrin (Molecular Partners AG, Zurich, Switzerland), domain antibodies (Domantis, Ltd., Cambridge, MA, and Ablynx nv, Zwijnaarde, Belgium), lipocalin (Pieris Proteolab AG, Freising, Germany), small modular immuno-pharmaceuticals (Trubion Pharmaceuticals Inc., Seattle, WA), maxybodies (Avidia, Inc., Mountain View, CA), Protein A (Affibody AG, Sweden), and affilin (gamma-crystallin or ubiquitin) (Scil Proteins GmbH, Halle, Germany).

Fibronectin scaffolds are typically based on fibronectin type III domain (e.g., the tenth module of the fibronectin type III (10 Fn3 domain)). The fibronectin type III domain has 7 or 8 beta strands which are distributed between two beta sheets, which themselves pack against each other to form the core of the protein, and further containing loops (analogous to CDRs) which connect the beta strands to each other and are solvent exposed. There are at least three such loops at each edge of the beta sheet sandwich, where the edge is the boundary of the protein perpendicular to the direction of the beta strands (see U.S. Pat. No. 6,818,418). Because of this structure, the non-immunoglobulin antibody mimics antigen binding properties that are similar in nature and affinity to those of antibodies. These scaffolds can be used in a loop randomization and shuffling strategy in vitro that is similar to the process of affinity maturation of antibodies in vivo. These fibronectin-based molecules can be used as scaffolds where the loop regions of the molecule can be replaced with CDRs of the invention using standard cloning techniques.

The ankyrin technology is based on using proteins with ankyrin derived repeat modules as scaffolds for bearing variable regions which can be used for binding to different targets. The ankyrin repeat module typically is a about 33 amino acid polypeptide consisting of two anti-parallel α-helices and a β-turn. Binding of the variable regions can be optimized by using ribosome display.

Avimers are used by nature for protein-protein interactions and in human over 250 proteins are structurally based on A-domains. Avimers consist of a number of different “A-domain” monomers (2-10) linked via amino acid linkers. Avimers can be created that can bind to the target antigen using the methodology described in, for example, U.S. Patent Application Publication Nos. 20040175756; 20050053973; 20050048512; and 20060008844.

Affibody affinity ligands are small, simple proteins composed of a three-helix bundle based on the scaffold of one of the IgG-binding domains of Protein A. Protein A is a surface protein from the bacterium Staphylococcus aureus. This scaffold domain consists of 58 amino acids, 13 of which are randomized to generate affibody libraries with a large number of ligand variants (See e.g., U.S. Pat. No. 5,831,012). Affibody molecules mimic antibodies, they have a molecular weight of 6 kDa, compared to the molecular weight of antibodies, which is 150 kDa. In spite of its small size, the binding site of affibody molecules is similar to that of an antibody.

Anticalins are known commercially, e.g., Pieris ProteoLab AG. They are derived from lipocalins, a widespread group of small and robust proteins that are usually involved in the physiological transport or storage of chemically sensitive or insoluble compounds. Several natural lipocalins occur in human tissues or body liquids. The protein architecture is reminiscent of immunoglobulins, with hypervariable loops on top of a rigid framework. However, in contrast with antibodies or their recombinant fragments, lipocalins are composed of a single polypeptide chain with 160 to 180 amino acid residues, being just marginally bigger than a single immunoglobulin domain. The set of four loops, which makes up the binding pocket, shows pronounced structural plasticity and tolerates a variety of side chains. The binding site can thus be reshaped in a proprietary process in order to recognize prescribed target molecules of different shape with high affinity and specificity. One protein of lipocalin family, the bilin-binding protein (BBP) of Pieris brassicae has been used to develop anticalins by mutagenizing the set of four loops. One example of a patent application describing anticalins is in PCT Publication No. WO 199916873.

Affilin molecules are small non-immunoglobulin proteins which are designed for specific affinities towards proteins and small molecules. New affilin molecules can be very quickly selected from two libraries, each of which is based on a different human derived scaffold protein. Affilin molecules do not show any structural homology to immunoglobulin proteins. Currently, two affilin scaffolds are employed, one of which is gamma crystalline, a human structural eye lens protein and the other is “ubiquitin” superfamily proteins. Both human scaffolds are very small, show high temperature stability and are almost resistant to pH changes and denaturing agents. This high stability is mainly due to the expanded beta sheet structure of the proteins. Examples of gamma crystalline derived proteins are described in WO200104144 and examples of “ubiquitin-like” proteins are described in WO2004106368.

Protein epitope mimetics (PEM) are medium-sized, cyclic, peptide-like molecules (MW 1-2 kDa) mimicking beta-hairpin secondary structures of proteins, the major secondary structure involved in protein-protein interactions.

Domain antibodies (dAbs) can be used in the anti-TCRvb antibody molecules as described herein or multifunctional formats thereof are small functional binding fragments of antibodies, corresponding to the variable regions of either the heavy or light chains of antibodies. Domain antibodies are well expressed in bacterial, yeast, and mammalian cell systems. Further details of domain antibodies and methods of production thereof are known in the art (see, for example, U.S. Pat. Nos. 6,291,158; 6,582,915; 6,593,081; 6,172,197; 6,696,245 European Patents 0368684 & 0616640; WO05/035572, WO04/101790, WO04/081026, WO04/058821, WO04/003019 and WO03/002609. Nanobodies are derived from the heavy chains of an antibody.

A nanobody typically comprises a single variable domain and two constant domains (CH2 and CH3) and retains antigen-binding capacity of the original antibody. Nanobodies can be prepared by methods known in the art (See e.g., U.S. Pat. Nos. 6,765,087, 6,838,254, WO 06/079372). Unibodies consist of one light chain and one heavy chain of an IgG4 antibody. Unibodies may be made by the removal of the hinge region of IgG4 antibodies. Further details of unibodies and methods of preparing them may be found in WO2007/059782.

### Anti-TCRVβAntibody or Anti-TCRVα Effector Function and Fc Variants

In some embodiments, an anti-TCRβ or anti-TCRVα antibody as described herein comprises an Fc region, e.g., as described herein. In some embodiments, the Fc region is a wildtype Fc region, e.g., a wildtype human Fc region. In some embodiments, the Fc region comprises a variant, e.g., an Fc region comprising an addition, substitution, or deletion of at least one amino acid residue in the Fc region which results in, e.g., reduced or ablated affinity for at least one Fc receptor.

The Fc region of an antibody interacts with a number of receptors or ligands including Fc Receptors (e.g., FcγRI, FcγRIIA, FcγRIIIA), the complement protein CIq, and other molecules such as proteins A and G. These interactions are essential for a variety of effector functions and downstream signaling events including: antibody dependent cell-mediated cytotoxicity (ADCC), Antibody-dependent cellular phagocytosis (ADCP) and complement dependent cytotoxicity (CDC).

In some embodiments, an anti-TCRVβ or anti-TCRva antibody comprising a variant Fc region has reduced, e.g., ablated, affinity for an Fc receptor, e.g., an Fc receptor described herein. In some embodiments, the reduced affinity is compared to an otherwise similar antibody with a wildtype Fc region.

In some embodiments, an anti-TCRVβ or anti-TCRva antibody comprising a variant Fc region has one or more of the following properties; (1) reduced effector function (e.g., reduced ADCC, ADCP and/or CDC); (2) reduced binding to one or more Fc receptors; and/or (3) reduced binding to C1q complement. In some embodiments, the reduction in any one, or all of properties (1)-(3) is compared to an otherwise similar antibody with a wildtype Fc region.

In some embodiments, an anti-TCRVβ or anti-TCRvα antibody comprising a variant Fc region has reduced affinity to a human Fc receptor, e.g., FcγR I, FcγR II and/or FcγR III. In some embodiments, the anti-TCRVβ or anti-TCRvα antibody comprising a variant Fc region comprises a human IgG1 region or a human IgG4 region.

In some embodiments, an anti-TCRVβ or anti-TCRvα antibody comprising a variant Fc region activates and/or expands T cells, e.g., as described herein. In some embodiments, an anti-TCRVβ or anti-TCRva antibody comprising a variant Fc region has a cytokine profile described herein, e.g., a cytokine profile that differs from a cytokine profile of a T cell engager that binds to a receptor or molecule other than a TCRβV region (“a non-TCRβV-binding T cell engager”) or a TCRαV region (“a non-TCRαV-binding T cell engager”). In some embodiments, the non-TCRβV-binding or non-TCRαV-binding T cell engager comprises an antibody that binds to a CD3 molecule (e.g., CD3 epsilon (CD3e) molecule); or a TCR alpha (TCRα) molecule.

Exemplary Fc region variants are provided in Table 14 and also disclosed in Saunders O, (2019) Frontiers in Immunology; vol 10, article 1296, the entire contents of which is hereby incorporated by reference.

In some embodiments, an anti-TCRVβ or anti-TCRVα antibody as described herein comprises any one or all, or any combination of Fc region variants disclosed in Table 14.

In some embodiments, an anti-TCRVβ or anti-TCRVα antibody as described herein comprises any one or all, or any combination of Fc region variants, e.g., mutations, disclosed in Table 14. In some embodiments, an anti-TCRVβ or anti-TCRVα antibody as described herein comprise an Asn297Ala (N297A) mutation. In some embodiments, an anti-TCRVβ antibody or anti-TCRVα as described herein comprise a Leu234Ala/Leu235Ala (LALA) mutation.

### Multifunctional Molecules

As used herein, a “multifunctional” or a “multispecific” molecule refers to molecule, e.g., a polypeptide, that has two or more functionalities, e.g., two or more binding specificities. In some embodiments, the functionalities can include one or more immune cell engagers, one or more tumor binding molecules, one or more cytokine molecules, one or more stromal modifiers, and other moieties described herein. In some embodiments, the multispecific molecule is a multispecific antibody molecule, e.g., a bispecific antibody molecule. In some embodiments, the multispecific molecule includes an anti-TCRVb antibody molecule as described herein.

Described herein, in certain embodiments, is a multifunctional polypeptide molecule comprising a first polypeptide, a second polypeptide, a third polypeptide, a fourth polypeptide, and at least one cytokine polypeptide or a functional fragment or a functional variant thereof, wherein the first polypeptide, the second polypeptide, the third polypeptide, and the fourth polypeptide are non-contiguous, wherein: (i) the first polypeptide comprising a first portion of a first T cell receptor variable beta (TCRβV)-binding moiety and a first dimerization module linked to the first portion of the first TCRβV-binding moiety; (ii) the second polypeptide comprising a second portion of the first TCRβV-binding moiety; (iii) the third polypeptide comprising a first portion of a second TCRβV-binding moiety and a second dimerization module linked to the first portion of the second TCRβV-binding moiety; and (iv) the fourth polypeptide comprising a second portion of the second TCRβV-binding moiety; and wherein the at least one cytokine polypeptide or a functional fragment or a functional variant thereof is covalently linked to the first polypeptide, the second polypeptide, the third polypeptide, the fourth polypeptide, or a combination thereof.

Described herein, in certain embodiments, is a multifunctional polypeptide molecule comprising a first polypeptide, a second polypeptide, a third polypeptide, and at least one cytokine polypeptide or a functional fragment or a functional variant thereof, wherein the first polypeptide, the second polypeptide, and the third polypeptide are non-contiguous, wherein: (i) the first polypeptide comprising a first portion of a first TCRβV-binding moiety and a first dimerization module linked to the first portion of the first TCRβV-binding moiety; (ii) the second polypeptide comprising a second portion of the first TCRβV-binding moiety; and (iii) the third polypeptide comprising a second dimerization module; and wherein the at least one cytokine polypeptide or a functional fragment or a functional variant thereof is covalently linked to the first polypeptide, the second polypeptide, the third polypeptide, or a combination thereof.

Described herein, in certain embodiments, is a multifunctional polypeptide molecule comprising a first polypeptide, a second polypeptide, a third polypeptide, and at least one cytokine polypeptide or a functional fragment or a functional variant thereof, wherein the first polypeptide, the second polypeptide, and the third polypeptide are non-contiguous, wherein: (i) the first polypeptide comprising a first portion of a first TCRβV-binding moiety and a first dimerization module linked to the first portion of the first TCRβV-binding moiety; (ii) the second polypeptide comprising a second portion of the first TCRβV-binding moiety; and (iii) the third polypeptide comprising a second dimerization module; wherein the at least one cytokine polypeptide or a functional fragment or a functional variant thereof is covalently linked to the first polypeptide, the second polypeptide, the third polypeptide, or a combination thereof; and wherein the multifunctional polypeptide molecule does not comprise an additional TCRβV-binding moiety except the first TCRβV-binding moiety.

In some embodiments, the first portion of the first TCRβV-binding moiety comprises a first heavy chain variable domain (VH) and a first heavy chain constant domain 1 (CH1) linked to the first VH. In some embodiments, the first CH1 is linked to the C-terminus of the first VH. In some embodiments, the second portion of the first TCRβV-binding moiety comprises a first light chain variable domain (VL) and a first light chain constant domain (CL) linked to the first VL. In some embodiments, first CL is linked to the C-terminus of the first VL. In some embodiments, wherein the first dimerization module is linked to the first portion of the first TCRβV-binding moiety. In some embodiments, the first dimerization module is linked to the C-terminus of the first portion of the first TCRβV-binding moiety. In some embodiments, wherein the first portion of the second TCRβV-binding moiety comprises a second VH and a second CH1 linked to the second VH. In some embodiments, the second CH1 is linked to the C-terminus of the second VH. In some embodiments, the second portion of the second TCRβV-binding moiety comprises a second VL and a second CL linked to the second VL. In some embodiments, the second CL is linked to the C-terminus of the second VL. In some embodiments, the second dimerization module is linked to the first portion of the second TCRβV-binding moiety. In some embodiments, the second dimerization module is linked to the C-terminus of the first portion of the second TCRβV-binding moiety.

In some embodiments, (a) the N-terminus of the first polypeptide is linked to a first cytokine polypeptide or a functional fragment or a functional variant thereof; the C-terminus of the first polypeptide is linked to a second cytokine polypeptide or a functional fragment or a functional variant thereof; or a combination thereof; (b) the N-terminus of the second polypeptide is linked to a third cytokine polypeptide or a functional fragment or a functional variant thereof; the C-terminus of the second polypeptide is linked to a fourth cytokine polypeptide or a functional fragment or a functional variant thereof; or a combination thereof; (c) the N-terminus of the third polypeptide is linked to a fifth cytokine polypeptide or a functional fragment or a functional variant thereof; the C-terminus of the third polypeptide is linked to a sixth cytokine polypeptide or a functional fragment or a functional variant thereof; or a combination thereof; (d) the N-terminus of the fourth polypeptide is linked to a seventh cytokine polypeptide or a functional fragment or a functional variant thereof; the C-terminus of the fourth polypeptide is linked to an eighth cytokine polypeptide or a functional fragment or a functional variant thereof; or a combination thereof; or (e) a combination thereof.

In some embodiments, (a-1) the N-terminus of the first polypeptide is linked to the first cytokine polypeptide or a functional fragment or a functional variant thereof; the C-terminus of the first polypeptide is linked to the second cytokine polypeptide or a functional fragment or a functional variant thereof; or a combination thereof; and (a-2) the N-terminus of the second polypeptide is linked to the third cytokine polypeptide or a functional fragment or a functional variant thereof; the C-terminus of the second polypeptide is linked to the fourth cytokine polypeptide or a functional fragment or a functional variant thereof; or a combination thereof; (b-1) the N-terminus of the first polypeptide is linked to the first cytokine polypeptide or a functional fragment or a functional variant thereof; the C-terminus of the first polypeptide is linked to the second cytokine polypeptide or a functional fragment or a functional variant thereof; or a combination thereof; and (b-2) the N-terminus of the third polypeptide is linked to the fifth cytokine polypeptide or a functional fragment or a functional variant thereof; the C-terminus of the third polypeptide is linked to the sixth cytokine polypeptide or a functional fragment or a functional variant thereof; or a combination thereof; (c-1) the N-terminus of the first polypeptide is linked to the first cytokine polypeptide or a functional fragment or a functional variant thereof; the C-terminus of the first polypeptide is linked to the second cytokine polypeptide or a functional fragment or a functional variant thereof; or a combination thereof; and (c-2) the N-terminus of the fourth polypeptide is linked to the seventh cytokine polypeptide or a functional fragment or a functional variant thereof; the C-terminus of the fourth polypeptide is linked to the eighth cytokine polypeptide or a functional fragment or a functional variant thereof; or a combination thereof; (d-1) the N-terminus of the second polypeptide is linked to the third cytokine polypeptide or a functional fragment or a functional variant thereof; the C-terminus of the second polypeptide is linked to the fourth cytokine polypeptide or a functional fragment or a functional variant thereof; or a combination thereof; and (d-2) the N-terminus of the third polypeptide is linked to the fifth cytokine polypeptide or a functional fragment or a functional variant thereof; the C-terminus of the third polypeptide is linked to the sixth cytokine polypeptide or a functional fragment or a functional variant thereof; or a combination thereof; (e-1) the N-terminus of the second polypeptide is linked to the third cytokine polypeptide or a functional fragment or a functional variant thereof; the C-terminus of the second polypeptide is linked to the fourth cytokine polypeptide or a functional fragment or a functional variant thereof; or a combination thereof; and (e-2) the N-terminus of the fourth polypeptide is linked to the seventh cytokine polypeptide or a functional fragment or a functional variant thereof; the C-terminus of the fourth polypeptide is linked to the eighth cytokine polypeptide or a functional fragment or a functional variant thereof; or a combination thereof; or (f-1) the N-terminus of the third polypeptide is linked to the fifth cytokine polypeptide or a functional fragment or a functional variant thereof; the C-terminus of the third polypeptide is linked to the sixth cytokine polypeptide or a functional fragment or a functional variant thereof; or a combination thereof; and (f-2) the N-terminus of the fourth polypeptide is linked to the seventh cytokine polypeptide or a functional fragment or a functional variant thereof; the C-terminus of the fourth polypeptide is linked to the eighth cytokine polypeptide or a functional fragment or a functional variant thereof; or a combination thereof.

In some embodiments, (a-1) the N-terminus of the first polypeptide is linked to the first cytokine polypeptide or a functional fragment or a functional variant thereof; the C-terminus of the first polypeptide is linked to the second cytokine polypeptide or a functional fragment or a functional variant thereof; or a combination thereof; (a-2) the N-terminus of the second polypeptide is linked to the third cytokine polypeptide or a functional fragment or a functional variant thereof; the C-terminus of the second polypeptide is linked to the fourth cytokine polypeptide or a functional fragment or a functional variant thereof; or a combination thereof; and (a-3) the N-terminus of the third polypeptide is linked to the fifth cytokine polypeptide or a functional fragment or a functional variant thereof; the C-terminus of the third polypeptide is linked to the sixth cytokine polypeptide or a functional fragment or a functional variant thereof; or a combination thereof; (b-1) the N-terminus of the first polypeptide is linked to the first cytokine polypeptide or a functional fragment or a functional variant thereof; the C-terminus of the first polypeptide is linked to the second cytokine polypeptide or a functional fragment or a functional variant thereof; or a combination thereof; (b-2) the N-terminus of the second polypeptide is linked to the third cytokine polypeptide or a functional fragment or a functional variant thereof; the C-terminus of the second polypeptide is linked to the fourth cytokine polypeptide or a functional fragment or a functional variant thereof; or a combination thereof; and (b-3) the N-terminus of the fourth polypeptide is linked to the seventh cytokine polypeptide or a functional fragment or a functional variant thereof; the C-terminus of the fourth polypeptide is linked to the eighth cytokine polypeptide or a functional fragment or a functional variant thereof; or a combination thereof; or (c-1) the N-terminus of the second polypeptide is linked to the third cytokine polypeptide or a functional fragment or a functional variant thereof; the C-terminus of the second polypeptide is linked to the fourth cytokine polypeptide or a functional fragment or a functional variant thereof; or a combination thereof; (c-2) the N-terminus of the third polypeptide is linked to the fifth cytokine polypeptide or a functional fragment or a functional variant thereof; the C-terminus of the third polypeptide is linked to the sixth cytokine polypeptide or a functional fragment or a functional variant thereof; or a combination thereof; and (c-3) the N-terminus of the fourth polypeptide is linked to the seventh cytokine polypeptide or a functional fragment or a functional variant thereof; the C-terminus of the fourth polypeptide is linked to the eighth cytokine polypeptide or a functional fragment or a functional variant thereof; or a combination thereof.

In some embodiments, (1) the N-terminus of the first polypeptide is linked to the first cytokine polypeptide or a functional fragment or a functional variant thereof; the C-terminus of the first polypeptide is linked to the second cytokine polypeptide or a functional fragment or a functional variant thereof; or a combination thereof; (2) the N-terminus of the second polypeptide is linked to the third cytokine polypeptide or a functional fragment or a functional variant thereof; the C-terminus of the second polypeptide is linked to the fourth cytokine polypeptide or a functional fragment or a functional variant thereof; or a combination thereof; (3) the N-terminus of the third polypeptide is linked to the fifth cytokine polypeptide or a functional fragment or a functional variant thereof; the C-terminus of the third polypeptide is linked to the sixth cytokine polypeptide or a functional fragment or a functional variant thereof; or a combination thereof; and (4) the N-terminus of the fourth polypeptide is linked to the seventh cytokine polypeptide or a functional fragment or a functional variant thereof; the C-terminus of the fourth polypeptide is linked to the eighth cytokine polypeptide or a functional fragment or a functional variant thereof; or a combination thereof.

In some embodiments, the first cytokine polypeptide, the second cytokine polypeptide, or a combination thereof is within a single contiguous polypeptide chain of the first polypeptide, the third cytokine polypeptide, the fourth cytokine polypeptide, or a combination thereof is within a single contiguous polypeptide chain of the second polypeptide, the fifth cytokine polypeptide, the sixth cytokine polypeptide, or a combination thereof is within a single contiguous polypeptide chain of the third polypeptide, the seventh cytokine polypeptide, the eighth cytokine polypeptide, or a combination thereof is within a single contiguous polypeptide chain of the fourth polypeptide, or a combination thereof.

In some embodiments, (a) the N-terminus of the first polypeptide is linked to a first cytokine polypeptide or a functional fragment or a functional variant thereof; the C-terminus of the first polypeptide is linked to a second cytokine polypeptide or a functional fragment or a functional variant thereof; or a combination thereof; (b) the N-terminus of the second polypeptide is linked to a third cytokine polypeptide or a functional fragment or a functional variant thereof; the C-terminus of the second polypeptide is linked to a fourth cytokine polypeptide or a functional fragment or a functional variant thereof; or a combination thereof; (c) the N-terminus of the third polypeptide is linked to a fifth cytokine polypeptide or a functional fragment or a functional variant thereof; the C-terminus of the third polypeptide is linked to a sixth cytokine polypeptide or a functional fragment or a functional variant thereof; or a combination thereof, or (d) a combination thereof.

In some embodiments, (a-1) the N-terminus of the first polypeptide is linked to the first cytokine polypeptide or a functional fragment or a functional variant thereof; the C-terminus of the first polypeptide is linked to the second cytokine polypeptide or a functional fragment or a functional variant thereof, or a combination thereof, and (a-2) the N-terminus of the second polypeptide is linked to the third cytokine polypeptide or a functional fragment or a functional variant thereof, the C-terminus of the second polypeptide is linked to the fourth cytokine polypeptide or a functional fragment or a functional variant thereof, or a combination thereof; (b-1) the N-terminus of the first polypeptide is linked to the first cytokine polypeptide or a functional fragment or a functional variant thereof; the C-terminus of the first polypeptide is linked to the second cytokine polypeptide or a functional fragment or a functional variant thereof, or a combination thereof; and (b-2) the N-terminus of the third polypeptide is linked to the fifth cytokine polypeptide or a functional fragment or a functional variant thereof; the C-terminus of the third polypeptide is linked to the sixth cytokine polypeptide or a functional fragment or a functional variant thereof, or a combination thereof; or (c-1) the N-terminus of the second polypeptide is linked to the third cytokine polypeptide or a functional fragment or a functional variant thereof, the C-terminus of the second polypeptide is linked to the fourth cytokine polypeptide or a functional fragment or a functional variant thereof; or a combination thereof; and (c-2) the N-terminus of the third polypeptide is linked to the fifth cytokine polypeptide or a functional fragment or a functional variant thereof; the C-terminus of the third polypeptide is linked to the sixth cytokine polypeptide or a functional fragment or a functional variant thereof, or a combination thereof.

In some embodiments, (1) the N-terminus of the first polypeptide is linked to the first cytokine polypeptide or a functional fragment or a functional variant thereof, the C-terminus of the first polypeptide is linked to the second cytokine polypeptide or a functional fragment or a functional variant thereof; or a combination thereof; (2) the N-terminus of the second polypeptide is linked to the third cytokine polypeptide or a functional fragment or a functional variant thereof; the C-terminus of the second polypeptide is linked to the fourth cytokine polypeptide or a functional fragment or a functional variant thereof, or a combination thereof, and (3) the N-terminus of the third polypeptide is linked to the fifth cytokine polypeptide or a functional fragment or a functional variant thereof, the C-terminus of the third polypeptide is linked to the sixth cytokine polypeptide or a functional fragment or a functional variant thereof, or a combination thereof.

In some embodiments, the first cytokine polypeptide, the second cytokine polypeptide, or a combination thereof is within a single contiguous polypeptide chain of the first polypeptide, the third cytokine polypeptide, the fourth cytokine polypeptide, or a combination thereof is within a single contiguous polypeptide chain of the second polypeptide, the fifth cytokine polypeptide, the sixth cytokine polypeptide, or a combination thereof is within a single contiguous polypeptide chain of the third polypeptide, or a combination thereof.

In some embodiments, the multifunctional polypeptide molecule as described herein further comprises a linker between the first portion of the first TCRβV-binding moiety and the first dimerization module, a linker between the first portion of the second TCRβV-binding moiety and the second dimerization module, a linker between the first VH and the first CH1, a linker between the first VL and the first CL, a linker between the second VH and the second CH1, a linker between the second VL and the second CL, a linker between the at least one cytokine polypeptide or a functional fragment or a functional variant thereof and the first polypeptide, a linker between the at least one cytokine polypeptide or a functional fragment or a functional variant thereof and the second polypeptide, a linker between the at least one cytokine polypeptide or a functional fragment or a functional variant thereof and the third polypeptide, a linker between the at least one cytokine polypeptide or a functional fragment or a functional variant thereof and the fourth polypeptide, or a combination thereof.

In some embodiments, the multifunctional polypeptide molecule as described herein further comprises comprising a linker between the first portion of the first TCRβV-binding moiety and the first dimerization module, a linker between the first VH and the first CH1, a linker between the first VL and the first CL, a linker between the at least one cytokine polypeptide or a functional fragment or a functional variant thereof and the first polypeptide, a linker between the at least one cytokine polypeptide or a functional fragment or a functional variant thereof and the second polypeptide, a linker between the at least one cytokine polypeptide or a functional fragment or a functional variant thereof and the third polypeptide, or a combination thereof. In some embodiments, linker is selected from the group consisting of a cleavable linker, a non-cleavable linker, a peptide linker, a flexible linker, a rigid linker, a helical linker, and a non-helical linker. In some embodiments, the linker is the peptide linker and wherein the linker is a GS linker. In some embodiments, the linker is the peptide linker and wherein the linker comprises the sequence of SEQ ID NO: 3308 or SEQ ID NO: 3643.

Described herein, in certain embodiments, is a multifunctional polypeptide molecule comprising a first polypeptide, a second polypeptide, a third polypeptide, a fourth polypeptide, a first cytokine polypeptide or a functional fragment or a functional variant thereof, and a second cytokine polypeptide or a functional fragment or a functional variant thereof, wherein the first polypeptide, the second polypeptide, the third polypeptide, and the fourth polypeptide are non-contiguous, wherein: (i) the first polypeptide comprising a first portion of a first TCRβV-binding moiety and a first dimerization module linked to the first portion of the first TCRβV-binding moiety; (ii) the second polypeptide comprising a second portion of the first TCRβV-binding moiety; (iii) the third polypeptide comprising a first portion of a second TCRβV-binding moiety and a second dimerization module linked to the first portion of the second TCRβV-binding moiety; and (iv) the fourth polypeptide comprising a second portion of the second TCRβV-binding moiety; and wherein the first cytokine polypeptide or a functional fragment or a functional variant thereof is covalently linked to the C-terminus of the second polypeptide, and the second cytokine polypeptide or a functional fragment or a functional variant thereof is covalently linked to the C-terminus of the fourth polypeptide.

Described herein, in certain embodiments, is a multifunctional polypeptide molecule comprising a first polypeptide, a second polypeptide, a third polypeptide, a fourth polypeptide, a cytokine polypeptide or a functional fragment or a functional variant thereof, wherein the first polypeptide, the second polypeptide, the third polypeptide, and the fourth polypeptide are non-contiguous, wherein: (i) the first polypeptide comprising a first portion of a first TCRβV-binding moiety and a first dimerization module linked to the first portion of the first TCRβV-binding moiety; (ii) the second polypeptide comprising a second portion of the first TCRβV-binding moiety; (iii) the third polypeptide comprising a first portion of a second TCRβV-binding moiety and a second dimerization module linked to the first portion of the second TCRβV-binding moiety; and (iv) the fourth polypeptide comprising a second portion of the second TCRβV-binding moiety; and wherein the cytokine polypeptide or a functional fragment or a functional variant thereof is covalently linked to the C-terminus of the second polypeptide or the C-terminus of the fourth polypeptide.

Described herein, in certain embodiments, is a multifunctional polypeptide molecule comprising a first polypeptide, a second polypeptide, a third polypeptide, a fourth polypeptide, a cytokine polypeptide or a functional fragment or a functional variant thereof, wherein the first polypeptide, the second polypeptide, the third polypeptide, and the fourth polypeptide are non-contiguous, wherein: (i) the first polypeptide comprising a first portion of a first TCRβV-binding moiety and a first dimerization module linked to the first portion of the first TCRβV-binding moiety; (ii) the second polypeptide comprising a second portion of the first TCRβV-binding moiety; (iii) the third polypeptide comprising a first portion of a second TCRβV-binding moiety and a second dimerization module linked to the first portion of the second TCRβV-binding moiety; and (iv) the fourth polypeptide comprising a second portion of the second TCRβV-binding moiety; and wherein the cytokine polypeptide or a functional fragment or a functional variant thereof is covalently linked to the C-terminus of the first polypeptide or the C-terminus of the third polypeptide.

Described herein, in certain embodiments, is a multifunctional polypeptide molecule comprising a first polypeptide, a second polypeptide, a third polypeptide, and a cytokine polypeptide or a functional fragment or a functional variant thereof, wherein the first polypeptide, the second polypeptide, and the third polypeptide are non-contiguous, wherein: (i) the first polypeptide comprising a first portion of a first TCRβV-binding moiety and a first dimerization module linked to the first portion of the first TCRβV-binding moiety; (ii) the second polypeptide comprising a second portion of the first TCRβV-binding moiety; and (iii) the third polypeptide comprising a second dimerization module; wherein the at least one cytokine polypeptide or a functional fragment or a functional variant thereof is covalently linked to the N terminus of the third polypeptide; and wherein the multifunctional polypeptide molecule does not comprise an additional TCRβV-binding moiety except the first TCRβV-binding moiety.

In some embodiments, the first portion of the first TCRβV-binding moiety comprises a first VH and a first CH1 linked to the first VH. In some embodiments, the first CH1 is linked to the C-terminus of the first VH.

In some embodiments, the second portion of the first TCRβV-binding moiety comprises a first VL and a first CL linked to the first VL. In some embodiments, first CL is linked to the C-terminus of the first VL.

In some embodiments, the first dimerization module is linked to the first portion of the first TCRβV-binding moiety. In some embodiments, the first dimerization module is linked to the C-terminus of the first portion of the first TCRβV-binding moiety. In some embodiments, the first portion of the second TCRβV-binding moiety comprises a second VH and a second CH1 linked to the second VH. In some embodiments, the second CH1 is linked to the C-terminus of the second VH. In some embodiments, the second portion of the second TCRβV-binding moiety comprises a second VL and a second CL linked to the second VL. In some embodiments, the second CL is linked to the C-terminus of the second VL. In some embodiments, the second dimerization module is linked to the first portion of the second TCRβV-binding moiety. In some embodiments, the second dimerization module is linked to the C-terminus of the first portion of the second TCRβV-binding moiety.

In some embodiments, the multifunctional polypeptide molecule as described herein further comprises a linker between the first portion of the first TCRβV-binding moiety and the first dimerization module, a linker between the first portion of the second TCRβV-binding moiety and the second dimerization module, a linker between the first VH and the first CH1, a linker between the first VL and the first CL, a linker between the second VH and the second CH1, a linker between the second VL and the second CL, a linker between the at least one cytokine polypeptide or a functional fragment or a functional variant thereof and the first polypeptide, a linker between the at least one cytokine polypeptide or a functional fragment or a functional variant thereof and the second polypeptide, a linker between the at least one cytokine polypeptide or a functional fragment or a functional variant thereof and the third polypeptide, a linker between the at least one cytokine polypeptide or a functional fragment or a functional variant thereof and the fourth polypeptide, or a combination thereof. In some embodiments, the multifunctional polypeptide molecule as described herein further comprises a linker between the first portion of the first TCRβV-binding moiety and the first dimerization module, a linker between the first VH and the first CH1, a linker between the first VL and the first CL, a linker between the at least one cytokine polypeptide or a functional fragment or a functional variant thereof and the third polypeptide, or a combination thereof. In some embodiments, linker is selected from the group consisting of a cleavable linker, a non-cleavable linker, a peptide linker, a flexible linker, a rigid linker, a helical linker, and a non-helical linker. In some embodiments, the linker is the peptide linker and wherein the linker is a GS linker. In some embodiments, the linker is the peptide linker and wherein the linker comprises the sequence of SEQ ID NO: 3308 or SEQ ID NO: 3643.

In some embodiments, the first TCRβV-binding moiety, the second TCRβV-binding moiety, or a combination thereof comprises any one selected from the group consisting of a Fab, F(ab′)2, Fv, a single chain Fv (scFv), a single domain antibody, a diabody (dAb), a camelid antibody and a combination thereof. In some embodiments, the first TCRβV-binding moiety, the second TCRβV-binding moiety, or a combination thereof comprises a scFv or a Fab.

In some embodiments, the multifunctional polypeptide molecule does not comprise an additional antigen-binding moiety except the TCRβV-binding moiety. In some embodiments, the multifunctional polypeptide molecule further comprise an additional antigen-binding moiety that is not the TCRβV-binding moiety.

Described herein, in certain embodiments, is a multifunctional polypeptide molecule comprising a first polypeptide, a second polypeptide, and at least one cytokine polypeptide or a functional fragment or a functional variant thereof, wherein the first polypeptide and the second polypeptide are non-contiguous, wherein: (i) the first polypeptide comprising a first TCRβV-binding moiety and a first dimerization module linked to the C-terminus of the first TCRβV-binding moiety, wherein the first TCRβV-binding moiety comprises a first VL and a first VH; and (ii) the second polypeptide comprising a second TCRβV-binding moiety and a second dimerization module linked to the C-terminus of the second TCRβV-binding moiety; wherein the at least one cytokine polypeptide or a functional fragment or a functional variant thereof is covalently linked to the first polypeptide, the second polypeptide, or a combination thereof, wherein the first TCRβV-binding moiety, the second TCRβV-binding moiety, or a combination thereof comprises a scFv; and wherein the multifunctional polypeptide molecule does not comprise an additional antigen-binding moiety except the first TCRβV-binding moiety and the second TCRβV-binding moiety.

Described herein, in certain embodiments, is a multifunctional polypeptide molecule comprising a first polypeptide, a second polypeptide, and at least one cytokine polypeptide or a functional fragment or a functional variant thereof, wherein the first polypeptide and the second polypeptide are non-contiguous, wherein: (i) the first polypeptide comprising a first TCRβV-binding moiety and a first dimerization module linked to the C-terminus of the first TCRβV-binding moiety, wherein the first TCRβV-binding moiety comprises a first VL and a first VH; and (ii) the second polypeptide comprising a second dimerization module; wherein the at least one cytokine polypeptide or a functional fragment or a functional variant thereof is covalently linked to the first polypeptide, the second polypeptide, or a combination thereof; wherein the first TCRβV-binding moiety comprises a scFv; wherein the multifunctional polypeptide molecule does not comprise an additional antigen-binding moiety except the first TCRβV-binding moiety; and wherein the multifunctional polypeptide molecule does not comprise an additional TCRβV-binding moiety except the first TCRβV-binding moiety.

In some embodiments, (a) the N-terminus of the first polypeptide is linked to a first cytokine polypeptide or a functional fragment or a functional variant thereof; the C-terminus of the first polypeptide is linked to a second cytokine polypeptide or a functional fragment or a functional variant thereof; or a combination thereof; (b) the N-terminus of the second polypeptide is linked to a third cytokine polypeptide or a functional fragment or a functional variant thereof; the C-terminus of the second polypeptide is linked to a fourth cytokine polypeptide or a functional fragment or a functional variant thereof; or a combination thereof; or (e) a combination thereof.

In some embodiments, the first cytokine polypeptide, the second cytokine polypeptide, or a combination thereof is within a single contiguous polypeptide chain of the first polypeptide, the third cytokine polypeptide, the fourth cytokine polypeptide, or a combination thereof is within a single contiguous polypeptide chain of the second polypeptide, or a combination thereof.

In some embodiments, the multifunctional polypeptide molecule as described herein further comprises a linker between the first TCRβV-binding moiety and the first dimerization module, a linker between the second TCRβV-binding moiety and the second dimerization module, a linker between the at least one cytokine polypeptide or a functional fragment or a functional variant thereof and the first polypeptide, a linker between the at least one cytokine polypeptide or a functional fragment or a functional variant thereof and the second polypeptide, or a combination thereof.

In some embodiments, the multifunctional polypeptide molecule as described herein further comprises a linker between the first TCRβV-binding moiety and the first dimerization module, a linker between the at least one cytokine polypeptide or a functional fragment or a functional variant thereof and the first polypeptide, a linker between the at least one cytokine polypeptide or a functional fragment or a functional variant thereof and the second polypeptide, or a combination thereof. In some embodiments, the linker is selected from the group consisting of a cleavable linker, a non-cleavable linker, a peptide linker, a flexible linker, a rigid linker, a helical linker, and a non-helical linker. In some embodiments, the linker is the peptide linker and wherein the linker is a GS linker. In some embodiments, the linker is the peptide linker and wherein the linker comprises the sequence of SEQ ID NO: 3308 or SEQ ID NO: 3643.

In some embodiments, the multifunctional polypeptide molecule comprises at least two of the cytokine polypeptides. In some embodiments, the multifunctional polypeptide molecule comprises at least three of the cytokine polypeptides. In some embodiments, the multifunctional polypeptide molecule comprises at least four of the cytokine polypeptides. In some embodiments, the multifunctional polypeptide molecule comprises at least five of the cytokine polypeptides. In some embodiments, the multifunctional polypeptide molecule comprises at least six of the cytokine polypeptides. In some embodiments, the multifunctional polypeptide molecule comprises at least seven of the cytokine polypeptides. In some embodiments, the multifunctional polypeptide molecule comprises at least eight of the cytokine polypeptides. In some embodiments, the multifunctional polypeptide molecule comprises two of the cytokine polypeptides. In some embodiments, the multifunctional polypeptide molecule comprises three of the cytokine polypeptides. In some embodiments, the multifunctional polypeptide molecule comprises four of the cytokine polypeptides. In some embodiments, the multifunctional polypeptide molecule comprises five of the cytokine polypeptides. In some embodiments, the multifunctional polypeptide molecule comprises six of the cytokine polypeptides. In some embodiments, the multifunctional polypeptide molecule comprises seven of the cytokine polypeptides. In some embodiments, the multifunctional polypeptide molecule comprises eight of the cytokine polypeptides. In some embodiments, the multifunctional polypeptide molecule comprises two of the cytokine polypeptide, each of which is linked to the first polypeptide and the second polypeptide; the first polypeptide and the third polypeptides; the first polypeptide and the fourth polypeptide; the second and the third polypeptide; the second polypeptide and the fourth polypeptide; or the third polypeptide and the fourth polypeptide, respectively. In some embodiments, the multifunctional polypeptide molecule comprises three of the cytokine polypeptides, each of which is linked to the first polypeptide, the second polypeptide, and the third polypeptide; the first polypeptide, the second polypeptide, and the fourth polypeptide; the first polypeptide, the third polypeptide, and the fourth polypeptide; or the second polypeptide, the third polypeptide, and the fourth polypeptide, respectively. In some embodiments, the multifunctional polypeptide molecule comprises four of the cytokine polypeptides, each of which is linked to the first polypeptide, the second polypeptide, the third polypeptide, and the fourth polypeptide, respectively. In some embodiments, the cytokine polypeptide is not linked to the polypeptides that comprise the first TCRβV-binding moiety.

In some embodiments, the at least one cytokine polypeptide is selected from the group consisting of interleukin-2 (IL-2) or a fragment or a functional fragment or a functional variant thereof, interleukin-7 (IL-7) or a fragment or a functional fragment or a functional variant thereof, interleukin-12 (IL-12) or a fragment or a functional fragment or a functional variant thereof, interleukin-15 (IL-15) or a fragment or a functional fragment or a functional variant thereof, interleukin-18 (IL-18) or a fragment or a functional fragment or a functional variant thereof, interleukin-21 (IL-21) or a fragment or a functional fragment or a functional variant thereof, or interferon gamma or a fragment or a functional fragment or a functional variant thereof, or a combination thereof.

In some embodiments, the at least one cytokine polypeptide comprises interleukin-2 (IL-2) or a fragment thereof. In some embodiments, the at least one cytokine polypeptide is interleukin-2 (IL-2) or a fragment thereof. In some embodiments, the at least one cytokine polypeptide comprises a sequence having at least 75%, 80%, 85%, 90%, 95%, 99%, 99.5%, 99.9%, or 100% sequence identity to the sequence of SEQ ID NO: 2191. In some embodiments, the at least one cytokine polypeptide comprises the sequence of SEQ ID NO: 2191. In some embodiments, the sequence of the at least one cytokine polypeptide is a sequence having at least 75%, 80%, 85%, 90%, 95%, 99%, 99.5%, 99.9/6, or 100% sequence identity to the sequence of SEQ ID NO: 2191. In some embodiments, the sequence of the at least one cytokine polypeptide is the sequence of SEQ ID NO: 2191.

In some embodiments, the variant of the at least one cytokine polypeptide comprises an IL-2 variant comprising a mutation. In some embodiments, the mutation comprises an insertion mutation, a deletion mutation, or a substitution mutation. In some embodiments, the mutation comprises the substitution mutation. In some embodiments, the variant comprises an IL-2 variant comprising C125A mutation. In some embodiments, the variant of the at least one cytokine polypeptide is an IL-2 variant comprising a mutation. In some embodiments, the mutation is an insertion mutation, a deletion mutation, or a substitution mutation. In some embodiments, the mutation is the substitution mutation. In some embodiments, the variant is an IL-2 variant comprising C125A mutation. In some embodiments, the variant comprises a sequence having at least 75%, 80%, 85%, 90%, 95%, 99%, 99.5%, 99.9%, or 100% sequence identity to the sequence of SEQ ID NO: 2270. In some embodiments, the variant comprises the sequence of SEQ ID NO: 2270. In some embodiments, the sequence of the variant is a sequence having at least 75%, 80%, 85%, 90%, 95%, 99%, 99.5%, 99.9%, or 100% sequence identity to the sequence of SEQ ID NO: 2270. In some embodiments, the sequence of the variant is the sequence of SEQ ID NO: 2270.

In some embodiments, the first dimerization module comprises a first immunoglobulin constant regions (Fc regions) and the second dimerization module comprises a second Fc region. In some embodiments, the first dimerization module is a first immunoglobulin constant regions (Fc regions) and the second dimerization module is a second Fc region.

In some embodiments, the first Fc region, the second Fc region, or a combination thereof is selected from an IgG1 Fc region or a fragment thereof, an IgG2 Fc region or a fragment thereof, an IgG3 Fc region or a fragment thereof, an IgGA1 Fc region or a fragment thereof, an IgGA2 Fc region or a fragment thereof, an IgG4 Fc region or a fragment thereof, an IgJ Fc region or a fragment thereof, an IgM Fc region or a fragment thereof, an IgD Fc region or a fragment thereof, and an IgE Fc region or a fragment thereof.

In some embodiments, the first Fc region, the second Fc region, or a combination thereof is selected from a human IgG1 Fc region or a fragment thereof, a human IgG2 Fc region or a fragment thereof, and a human IgG4 Fc region or a fragment thereof.

In some embodiments, the first Fc region, the second Fc region, or a combination thereof comprises an Fc interface with one or more of: a paired cavity-protuberance, an electrostatic interaction, or a strand-exchange, wherein the dimerization of the first Fc region and the second Fc region is enhanced as indicated by a greater ratio of heteromultimer:homomultimer forms relative to a dimerization of Fc regions with a non-engineered interface. In some embodiments, the dimerization of the first Fc region and the second Fc region is enhanced at least by 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 150 fold, 200 fold, 250 fold, 300 fold, 250 fold, 400 fold, 450 fold, 500 fold, 550 fold, 600 fold, 650 fold, 700 fold, 750 fold, 800 fold, 850 fold, 900 fold, 950 fold, 1000 fold, 2000 fold, 3000 fold, 4000 fold, 5000 fold, 6000 fold, 7000 fold, 8000 fold, 9000 fold, or 10000 fold relative to a dimerization of Fc regions with a non-engineered interface. In some embodiments, the dimerization of the first Fc region and the second Fc region is enhanced at most by 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 150 fold, 200 fold, 250 fold, 300 fold, 250 fold, 400 fold, 450 fold, 500 fold, 550 fold, 600 fold, 650 fold, 700 fold, 750 fold, 800 fold, 850 fold, 900 fold, 950 fold, 1000 fold, 2000 fold, 3000 fold, 4000 fold, 5000 fold, 6000 fold, 7000 fold, 8000 fold, 9000 fold, or 10000 fold relative to a dimerization of Fc regions with a non-engineered interface. In some embodiments, the dimerization of the first Fc region and the second Fc region is enhanced by 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 150 fold, 200 fold, 250 fold, 300 fold, 250 fold, 400 fold, 450 fold, 500 fold, 550 fold, 600 fold, 650 fold, 700 fold, 750 fold, 800 fold, 850 fold, 900 fold, 950 fold, 1000 fold, 2000 fold, 3000 fold, 4000 fold, 5000 fold, 6000 fold, 7000 fold, 8000 fold, 9000 fold, or 10000 fold relative to a dimerization of Fc regions with a non-engineered interface.

In some embodiments, the first Fc region, the second Fc region, or a combination thereof comprises an amino acid substitution listed in Table 14.

In some embodiments, the first Fc region, the second Fc region, or a combination thereof comprises an Asn297Ala (N297A) mutation or a Leu234Ala/Leu235Ala (LALA) mutation.

In some embodiments, the first Fc region, the second Fc region, or a combination thereof comprises a sequence having at least 75%, 80%, 85%, 90%, 95%, 99%, 99.5%, 99.9%, or 100% sequence identity to the sequence of SEQ ID NO: 40, SEQ ID NO: 42, SEQ ID NO: 3645, SEQ ID NO: 3646, SEQ ID NO: 3647, SEQ ID NO:3648, or SEQ ID NO: 3649. In some embodiments, the first Fc region, the second Fc region, or a combination thereof comprises the sequence of SEQ ID NO: 40, SEQ ID NO: 42, SEQ ID NO: 3645, SEQ ID NO: 3646, SEQ ID NO: 3647, SEQ ID NO:3648, or SEQ ID NO: 3649.

In some embodiments, the sequence of the first Fc region, the second Fc region, or a combination thereof is a sequence having at least 75%, 80%, 85%, 90%, 95%, 99%, 99.5%, 99.9%, or 100% sequence identity to the sequence of SEQ ID NO: 40, SEQ ID NO: 42, SEQ ID NO: 3645, SEQ ID NO: 3646, SEQ ID NO: 3647, SEQ ID NO:3648, or SEQ ID NO: 3649. In some embodiments, the sequence of the first Fc region, the second Fc region, or a combination thereof is the sequence of SEQ ID NO: 40, SEQ ID NO: 42, SEQ ID NO: 3645, SEQ ID NO: 3646, SEQ ID NO: 3647, SEQ ID NO:3648, or SEQ ID NO: 3649.

In some embodiments, the first TCRβV-binding moiety, the second TCRβV-binding moiety, or a combination thereof binds to one or more of a TCRβV subfamily selected from the group consisting of: (i) TCRβ V5 subfamily comprising one or more selected from TCRβ V5-6*01, TCRβ V5-4*01, TCRβ V5-1*01, and TCRβ V5-8*01; (ii) the TCRβ V6 subfamily comprising one or more selected from TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01, and TCRβ V6-1*01; (iii) TCRβ V7 subfamily comprising one or more selected from TCRβ V7-1*01, TCRβ V7-2*01, TCRβ V7-2*02, TCRβ V2-1*03, TCRβ V7-2*04, TCRβ V7-3*01, TCRβ V7-3*02, TCRβ V7-3*03, TCRβ V7-3*04, TCRβ V7-3*05, TCRβ V7-4*01, TCRβ V7-4*02, TCRβ V7-5*01, TCRβ V7-5*02, TCRβ V7-6*01, TCRβ V7-6*02, TCRβ V7-7*01, TCRβ V7-7*02, TCRβ V7-8*01, TCRβ V7-8*02, TCRβ V7-8*03, TCRβ V7-9*01, TCRβ V7-9*02, TCRβV7-9*03, TCRβ V7-9*04, TCRβ V7-9*05, TCRβ V7-9*06, and TCRβ V7-9*07; (iv) TCRβ V10 subfamily comprising one or more selected from TCRβ V10-1*01, TCRβ V10-1*02, TCRβ V10-3*01, and TCRβ V10-2*01; (v) TCRβ V12 subfamily comprising one or more selected from TCRβ V12-4*01, TCRβ V12-3*01, and TCRβ V12-5*01; (vi) TCRβ V15 subfamily one or more selected from comprising TCRβ V15*01, TCRβ V15*02, and TCRβ V15*03; (vii) TCRβ V19 subfamily comprising one or more selected from TCRβ V19*01 and TCRβ V19*02; (viii) TCRβ V20 subfamily comprising one or more selected from TCRβ V20-1*01, TCRβ V20-1*02, TCRβ V20-1*03, TCRβ V20-1*04, TCRβ V20-1*05, TCRβ V20-1*06, and TCRβ V20-1*07; and (ix) TCRβ V28 subfamily.

In some embodiments, the first TCRβV-binding moiety and the second TCRβV-binding moiety are same. In some embodiments, the first TCRβV-binding moiety and the second TCRβV-binding moiety are different.

In some embodiments, the first TCRβV-binding moiety and the second TCRβV-binding moiety binds: (i) one or more of a TCRβ V6 subfamily member and one or more of a TCRβ V10 subfamily member, respectively; (ii) one or more of a TCRβ V6 subfamily member and one or more of a TCRβ V5 subfamily member, respectively; (iii) one or more of a TCRβ V6 subfamily member and one or more of a TCRβ V12 subfamily member, respectively; (iv) one or more of a TCRβ V10 subfamily member and one or more of a TCRβ V5 subfamily member, respectively; (v) one or more of a TCRβ V10 subfamily member and one or more of a TCRβ V12 subfamily member, respectively; or (vi) one or more of a TCRβ V5 subfamily member and one or more of a TCRβ V12 subfamily member, respectively.

In some embodiments, the first TCRβV-binding moiety, the second TCRβV-binding moiety, or a combination thereof comprises: (i) a HC CDR1, a HC CDR2 and a HC CDR3 of an amino acid sequence having at least 75%, 80%, 85%, 90%, 95%, 99%, 99.5%, 99.9%, or 100% sequence identity to any one of the CDR1, CDR2, and CDR3 sequences listed in Table 1; (ii) a LC CDR1, a LC CDR2, and a LC CDR3 of an amino acid sequence having at least 75%, 80%, 85%, 90%, 95%, 99%, 99.5%, 99.9%, or 100% sequence identity to any one of the CDR1, CDR2, and CDR3 the sequences listed in Table 1; or (iii) a combination thereof. In some embodiments, the first TCRβV-binding moiety, the second TCRβV-binding moiety, or a combination thereof comprises: (i) a HC CDR1, a HC CDR2 and a HC CDR3 having any one of the CDR1, CDR2, and CDR3 sequences listed in Table 1; (ii) a LC CDR1, a LC CDR2, and a LC CDR3 having any one of the CDR1, CDR2, and CDR3 the sequences listed in Table 1; or (iii) a combination thereof.

In some embodiments, the first TCRβV-binding moiety, the second TCRβV-binding moiety, or a combination thereof comprises: (i) a HC CDR1, a HC CDR2 and a HC CDR3 of an amino acid sequence having at least 75%, 80%, 85%, 90%, 95%, 99%, 99.5%, 99.9%, or 100% sequence identity to any one of the CDR1, CDR2, and CDR3 sequences listed in Table 1, respectively; (ii) a LC CDR1, a LC CDR2, and a LC CDR3 of an amino acid sequence having at least 75%, 80%, 85%, 90%, 95%, 99%, 99.5%, 99.9%, or 100% sequence identity to any one of the CDR1, CDR2, and CDR3 the sequences listed in Table 1, respectively; or (iii) a combination thereof. In some embodiments, the first TCRβV-binding moiety, the second TCRβV-binding moiety, or a combination thereof comprises: (i) a HC CDR1, a HC CDR2 and a HC CDR3 having any one of the CDR1, CDR2, and CDR3 sequences listed in Table 1, respectively; (ii) a LC CDR1, a LC CDR2, and a LC CDR3 having any one of the CDR1, CDR2, and CDR3 the sequences listed in Table 1, respectively; or (iii) a combination thereof.

In some embodiments, the first TCRβV-binding moiety, the second TCRβV-binding moiety, or a combination thereof comprises: (i) a VH comprising a framework region (FR) comprising a framework 1 (FR1), a framework region 2 (FR2), a framework region 3 (FR3), and a framework region 4 (FR4) that have at least 75%, 80%, 85%, 90%, 95%, 99%, 99.5%, 99.9%, or 100% sequence identity with a non-murine germline FR1, a non-murine germline FR2, a non-murine germline FR3, and a non-murine germline FR4; (ii) a VL comprising a FR comprising a FR1, a FR2, a FR3, and a FR4 that have at least 75%, 80%, 85%, 90%, 95%, 99%, 99.5%, 99.9%, or 100% sequence identity with a non-murine germline FR1, a non-murine germline FR2, a non-murine germline FR3, and a non-murine germline FR4; or (iii) a combination thereof. In some embodiments, the first TCRβV-binding moiety, the second TCRβV-binding moiety, or a combination thereof comprises: (i) a VH comprising a FR comprising a FR1, a FR2, a FR3, and a FR4 having the sequences of a non-murine germline FR1, a non-murine germline FR2, a non-murine germline FR3, and a non-murine germline FR4; (ii) a VL comprising a FR comprising a FR1, a FR2, a FR3, and a FR4 having the sequences of a non-murine germline FR1, a non-murine germline FR2, a non-murine germline FR3, and a non-murine germline FR4; or (iii) a combination thereof.

In some embodiments, the first TCRβV-binding moiety, the second TCRβV-binding moiety, or a combination thereof comprises: (i) a VH comprising a FR1, a FR2, a FR3, and a FR4 that have at least 75%, 80%, 85%, 90%, 95%, 99%, 99.5%, 99.9%, or 100% sequence identity with a non-murine germline FR1, a non-murine germline FR2, a non-murine germline FR3, and a non-murine germline FR4, respectively; (ii) a VL comprising a FR comprising a FR1, a FR2, a FR3, and a FR4 that have at least 75%, 80%, 85%, 90%, 95%, 99%, 99.5%, 99.9%, or 100% sequence identity with a non-murine germline FR1, a non-murine germline FR2, a non-murine germline FR3, and a non-murine germline FR4, respectively; or (iii) a combination thereof. In some embodiments, the first TCRβV-binding moiety, the second TCRβV-binding moiety, or a combination thereof comprises: (i) a VH comprising a FR comprising a FR1, a FR2, a FR3, and a FR4 having the sequences of a non-murine germline FR1, a non-murine germline FR2, a non-murine germline FR3, and a non-murine germline FR4, respectively; (ii) a VL comprising a FR comprising a FR1, a FR2, a FR3, and a FR4 having the sequences of a non-murine germline FR1, a non-murine germline FR2, a non-murine germline FR3, and a non-murine germline FR4, respectively; or (iii) a combination thereof.

In some embodiments, the VH comprises the FR3 comprising (i) a Threonine at position 73 according to Kabat numbering; (ii) a Glycine a position 94 according to Kabat numbering; or (iii) a combination thereof. In some embodiments, the VL comprises the FR1 comprising a Phenyalanine at position 10 according to Kabat numbering. In some embodiments, the VL comprises the FR2 comprising (i) a Histidine at position 36 according to Kabat numbering; (ii) an Alanine at position 46 according to Kabat numbering; or (iii) a combination thereof. In some embodiments, the VL comprises the FR3 comprising a Phenyalanine at position 87 according to Kabat numbering.

In some embodiments, the first TCRβV-binding moiety, the second TCRβV-binding moiety, or a combination thereof comprises: (i) a HC CDR1, a HC CDR2 and a HC CDR3 of an amino acid sequence having at least 75%, 80%, 85%, 90%, 95%, 99%, 99.5%, 99.9%, or 100% sequence identity to any one of the CDR1, CDR2, and CDR3 sequences listed in Table 2; (ii) a LC CDR1, a LC CDR2, and a LC CDR3 of an amino acid sequence having at least 75%, 80%, 85%, 90%, 95%, 99%, 99.5%, 99.9%, or 100% sequence identity to any one of the CDR1, CDR2, and CDR3 sequences listed in Table 2; or (iii) a combination thereof. In some embodiments, the first TCRβV-binding moiety, the second TCRβV-binding moiety, or a combination thereof comprises: (i) a HC CDR1, a HC CDR2 and a HC CDR3 having any one of the CDR1, CDR2, and CDR3 sequences listed in Table 2; (ii) a LC CDR1, a LC CDR2, and a LC CDR3 having any one of the CDR1, CDR2, and CDR3 sequences listed in Table 2; or (iii) a combination thereof.

In some embodiments, the first TCRβV-binding moiety, the second TCRβV-binding moiety, or a combination thereof comprises: (i) a HC CDR1, a HC CDR2 and a HC CDR3 of an amino acid sequence having at least 75%, 80%, 85%, 90%, 95%, 99%, 99.5%, 99.9%, or 100% sequence identity to any one of the CDR1, CDR2, and CDR3 sequences listed in Table 2, respectively; (ii) a LC CDR1, a LC CDR2, and a LC CDR3 of an amino acid sequence having at least 75%, 80%, 85%, 90%, 95%, 99%, 99.5%, 99.9%, or 100% sequence identity to any one of the CDR1, CDR2, and CDR3 sequences listed in Table 2, respectively; or (iii) a combination thereof. In some embodiments, the first TCRβV-binding moiety, the second TCRβV-binding moiety, or a combination thereof comprises: (i) a HC CDR1, a HC CDR2 and a HC CDR3 having any one of the CDR1, CDR2, and CDR3 sequences listed in Table 2, respectively; (ii) a LC CDR1, a LC CDR2, and a LC CDR3 having any one of the CDR1, CDR2, and CDR3 sequences listed in Table 2, respectively; or (iii) a combination thereof.

In some embodiments, the first TCRβV-binding moiety, the second TCRβV-binding moiety, or a combination thereof comprises: (i) a VH comprising a FR comprising a FR1, a FR2, a FR3, and a FR4 that have at least 75%, 80%, 85%, 90%, 95%, 99%, 99.5%, 99.9%, or 100% sequence identity with a FR1, a FR2, a FR3, and a FR4 of a humanized B-H LC of Table 2; (ii) a VL comprising a FR comprising a FR1, a FR2, a FR3, and a FR4 that have at least 75%, 80%, 85%, 90%, 95%, 99%, 99.5%, 99.9%, or 100% sequence identity with a FR1, a FR2, a FR3, and a FR4 of a humanized B-H LC of Table 2; or (iii) a combination thereof. In some embodiments, the first TCRβV-binding moiety, the second TCRβV-binding moiety, or a combination thereof comprises: (i) a VH comprising a FR comprising a FR1, a FR2, a FR3, and a FR4 that have the sequence of a FR1, a FR2, a FR3, and a FR4 of a humanized B-H LC of Table 2; (ii) a VL comprising a FR comprising a FR1, a FR2, a FR3, and a FR4 that have the sequence of a FR1, a FR2, a FR3, and a FR4 of a humanized B-H LC of Table 2; or (iii) a combination thereof.

In some embodiments, the first TCRβV-binding moiety, the second TCRβV-binding moiety, or a combination thereof comprises: (i) a VH comprising a FR comprising a FR1, a FR2, a FR3, and a FR4 that have at least 75%, 80%, 85%, 90%, 95%, 99%, 99.5%, 99.9%, or 100% sequence identity with a FR1, a FR2, a FR3, and a FR4 of a humanized B-H LC of Table 2, respectively; (ii) a VL comprising a FR comprising a FR1, a FR2, a FR3, and a FR4 that have at least 75%, 80%, 85%, 90%, 95%, 99%, 99.5%, 99.9%, or 100% sequence identity with a FR1, a FR2, a FR3, and a FR4 of a humanized B-H LC of Table 2, respectively; or (iii) a combination thereof. In some embodiments, the first TCRβV-binding moiety, the second TCRβV-binding moiety, or a combination thereof comprises: (i) a VH comprising a FR comprising a FR1, a FR2, a FR3, and a FR4 that have the sequence of a FR1, a FR2, a FR3, and a FR4 of a humanized B-H LC of Table 2, respectively; (ii) a VL comprising a FR comprising a FR1, a FR2, a FR3, and a FR4 that have the sequence of a FR1, a FR2, a FR3, and a FR4 of a humanized B-H LC of Table 2, respectively; or (iii) a combination thereof.

In some embodiments, the first TCRβV-binding moiety, the second TCRβV-binding moiety, or a combination thereof comprises: (i) a VH comprising a sequence having at least 75%, 80%, 85%, 90%, 95%, 99%, 99.5%, 99.9%, or 100% sequence identity to the VH sequence of a humanized Antibody B-H listed in Table 2; (ii) a VL comprising a sequence having at least at least 75%, 80%, 85%, 90%, 95%, 99%, 99.5%, 99.9%, or 100% sequence identity to the VL sequence of a humanized Antibody B-H listed in Table 2, or (iii) a combination thereof. In some embodiments, the first TCRβV-binding moiety, the second TCRβV-binding moiety, or a combination thereof comprises: (i) a VH comprising the VH sequence of a humanized Antibody B-H listed in Table 2; (ii) a VL comprising the VL sequence of a humanized Antibody B-H listed in Table 2; or (iii) a combination thereof. In some embodiments, the first TCRβV-binding moiety, the second TCRβV-binding moiety, or a combination thereof comprises: (i) the VH of a humanized Antibody B-H listed in Table 2; (ii) the VL sequence of a humanized Antibody B-H listed in Table 2; or (iii) a combination thereof.

In some embodiments, the first polypeptide, the second polypeptide, the third polypeptide, the fourth polypeptide, or a combination thereof comprises a heavy chain constant region having a sequence having at least 75%, 80%, 85%, 90%, 95%, 99%, 99.5%, 99.9%, or 100% sequence identity to any one of the sequences listed in Table 3 or a combination thereof. In some embodiments, the first polypeptide, the second polypeptide, the third polypeptide, the fourth polypeptide, or a combination thereof comprises a heavy chain constant region having any one of the sequences listed in Table 3 or a combination thereof. In some embodiments, the first polypeptide, the second polypeptide, the third polypeptide, the fourth polypeptide, or a combination thereof comprises a heavy chain constant region of which sequence is a sequence having at least 75%, 80%, 85%, 90%, 95%, 99%, 99.5%, 99.9%, or 100% sequence identity to any one of the sequences listed in Table 3 or a combination thereof. In some embodiments, the first polypeptide, the second polypeptide, the third polypeptide, the fourth polypeptide, or a combination thereof comprises a heavy chain constant region having any one of the heavy chain constant region sequences listed in Table 3 or a combination thereof. In some embodiments, the first polypeptide, the second polypeptide, the third polypeptide, the fourth polypeptide, or a combination thereof comprises a heavy chain constant region of an IgM or a fragment thereof. In some embodiments, the heavy chain constant region of the IgM comprises a sequence having at least 75%, 80%, 85%, 90%, 95%, 99%, 99.5%, 99.9%, or 100% sequence identity to the sequence of SEQ ID NO: 73. In some embodiments, the heavy chain constant region of the IgM comprises the sequence of SEQ ID NO: 73. In some embodiments, the sequence of the heavy chain constant region of the IgM is the sequence of SEQ ID NO: 73.

In some embodiments, the first polypeptide, the second polypeptide, the third polypeptide, the fourth polypeptide, or a combination thereof comprises a heavy chain constant region of an IgJ or a fragment thereof. In some embodiments, the heavy chain constant region of the IgJ comprises a sequence having at least 75%, 80%, 85%, 90%, 95%, 99%, 99.5%, 99.9%, or 100% sequence identity to the sequence of SEQ ID NO: 76. In some embodiments, the heavy chain constant region of the IgJ comprises the sequence of SEQ ID NO: 76. In some embodiments, the sequence of the heavy chain constant region of the IgJ is the sequence of SEQ ID NO: 76.

In some embodiments, the first polypeptide, the second polypeptide, the third polypeptide, the fourth polypeptide, or a combination thereof comprises a heavy chain constant region of an IgGA1 or a fragment thereof. In some embodiments, the heavy chain constant region of the IgGA1 comprises a sequence having at least 75%, 80%, 85%, 90%, 95%, 99%, 99.5%, 99.9%, or 100% sequence identity to the sequence of SEQ ID NO: 74. In some embodiments, the heavy chain constant region of the IgGA1 comprises the sequence of SEQ ID NO: 74. In some embodiments, the sequence of the heavy chain constant region of the IgGA1 is the sequence of SEQ ID NO: 74.

In some embodiments, the first polypeptide, the second polypeptide, the third polypeptide, the fourth polypeptide, or a combination thereof comprises a heavy chain constant region of an IgGA2 or a fragment thereof. In some embodiments, the heavy chain constant region of the IgGA2 comprises a sequence having at least 75%, 80%, 85%, 90%, 95%, 99%, 99.5%, 99.9%, or 100% sequence identity to the sequence of SEQ ID NO: 75. In some embodiments, the heavy chain constant region of the IgGA2 comprises the sequence of SEQ ID NO: 75. In some embodiments, the sequence of the heavy chain constant region of the IgGA2 is the sequence of SEQ ID NO: 75.

In some embodiments, the first polypeptide, the second polypeptide, the third polypeptide, the fourth polypeptide, or a combination thereof comprises a heavy chain constant region of an IgG1 or a fragment thereof. In some embodiments, the heavy chain constant region of the IgG1 comprises a sequence having at least 75%, 80%, 85%, 90%, 95%, 99%, 99.5%, 99.9%, or 100% sequence identity to the sequence of SEQ ID NO: 41. In some embodiments, the heavy chain constant region of the IgG1 comprises the sequence of SEQ ID NO: 41. In some embodiments, the sequence of the heavy chain constant region of the IgG1 is the sequence of SEQ ID NO: 41. In some embodiments, the heavy chain constant region of the IgG1 comprises a sequence having at least 75%, 80%, 85%, 90%, 95%, 99%, 99.5%, 99.9%, or 100/6 sequence identity to the sequence of SEQ ID NO: 3645. In some embodiments, the heavy chain constant region of the IgG1 comprises the sequence of SEQ ID NO: 3645. In some embodiments, the sequence of the heavy chain constant region of the IgG1 is the sequence of SEQ ID NO: 3645.

In some embodiments, the first polypeptide, the second polypeptide, the third polypeptide, the fourth polypeptide, or a combination thereof comprises a light chain constant region having a sequence having at least 75%, 80%, 85%, 90%, 95%, 99%, 99.5%, 99.9%, or 100% sequence identity to any one of the sequences listed in Table 3 or a combination thereof. In some embodiments, the first polypeptide, the second polypeptide, the third polypeptide, the fourth polypeptide, or a combination thereof comprises a light chain constant region having any one of the sequences listed in Table 3 or a combination thereof. In some embodiments, the first polypeptide, the second polypeptide, the third polypeptide, the fourth polypeptide, or a combination thereof comprises a light chain constant region having any one of the light chain constant region sequences listed in Table 3 or a combination thereof.

In some embodiments, the first polypeptide, the second polypeptide, the third polypeptide, the fourth polypeptide, or a combination thereof comprises a light chain constant region of a kappa chain or a fragment thereof. In some embodiments, the light chain constant region of a kappa chain comprises a light chain constant region sequence listed in Table 3.

In some embodiments, the light chain constant region of a kappa chain comprises a sequence having at least 75%, 80%, 85%, 90%, 95%, 99%, 99.5%, 99.9%, or 100% sequence identity to the sequence of SEQ ID NO: 39 or SEQ ID NO: 3644. In some embodiments, the light chain constant region of a kappa chain comprises the sequence of SEQ ID NO: 39 or SEQ ID NO: 3644. In some embodiments, the sequence of the light chain constant region of a kappa chain is a sequence having at least 75%, 80%, 85%, 90%, 95%, 99%, 99.5%, 99.9%, or 100% sequence identity to the sequence of SEQ ID NO: 39 or SEQ ID NO: 3644. In some embodiments, the sequence of the light chain constant region of a kappa chain is the sequence of SEQ ID NO: 39 or SEQ ID NO: 3644.

In some embodiments, the first TCRβV-binding moiety, the second TCRβV-binding moiety, or a combination thereof comprises: (i) a HC CDR1, a HC CDR2 and a HC CDR3 comprising amino acid sequences having at least 75%, 80%, 85%, 90%, 95%, 99%, 99.5%, 99.9%, or 100% sequence identity to CDR1, CDR2, and CDR3 sequences of a VH disclosed in Tables 1, 2, 10, 11, 12 or 13; (ii) a LC CDR1, a LC CDR2, and a LC CDR3 comprising an amino acid sequence having at least 75%, 80%, 85%, 90%, 95%, 99%, 99.5%, 99.9%, or 100/a sequence identity to CDR1, CDR2, and CDR3 sequences of a VL disclosed in Tables 1, 2, 10, 11, 12 or 13, or (iii) a combination thereof. In some embodiments, the first TCRβV-binding moiety, the second TCRβV-binding moiety, or a combination thereof comprises: (i) a HC CDR1, a HC CDR2 and a HC CDR3 comprising the CDR1. CDR2, and CDR3 sequences of a VH disclosed in Tables 1, 2, 10, 11, 12 or 13, (ii) a LC CDR1, a LC CDR2, and a LC CDR3 comprising the CDR1, CDR2, and CDR3 sequences of a VL disclosed in Tables 1, 2, 10, 11, 12 or 13; or (iii) a combination thereof. In some embodiments, the first TCRβV-binding moiety, the second TCRβV-binding moiety, or a combination thereof comprises: (i) a HC CDR1, a HC CDR2 and a HC CDR3 comprising amino acid sequences having at least 75%, 80%, 85%, 90%, 95%, 99%, 99.5%, 99.9%, or 100% sequence identity to CDR1, CDR2, and CDR3 sequences of a VH disclosed in Tables 1, 2, 10, 11, 12 or 13, respectively; (ii) a LC CDR1, a LC CDR2, and a LC CDR3 comprising an amino acid sequence having at least 75%, 80%, 850, 90%, 95%, 99%, 99.5%, 99.9%, or 100% sequence identity to CDR1, CDR2, and CDR3 sequences of a VL disclosed in Tables 1, 2, 10, 11, 12 or 13, respectively; or (iii) a combination thereof. In some embodiments, the first TCRβV-binding moiety, the second TCRβV-binding moiety, or a combination thereof comprises: (i) a HC CDR1, a HC CDR2 and a HC CDR3 comprising the CDR1, CDR2, and CDR3 sequences of a VH disclosed in Tables 1, 2, 10, 11, 12 or 13, respectively; (ii) a LC CDR1, a LC CDR2, and a LC CDR3 comprising the CDR1, CDR2, and CDR3 sequences of a VL disclosed in Tables 1, 2, 10, 11, 12 or 13, respectively; or (iii) a combination thereof. In some embodiments, the first TCRβV-binding moiety, the second TCRβV-binding moiety, or a combination thereof comprises: (i) a HC CDR1, a HC CDR2 and a HC CDR3 of a VH disclosed in Tables 1, 2, 10, 11, 12 or 13; (ii) a LC CDR1, a LC CDR2, and a LC CDR3 of a VL disclosed in Tables 1, 2, 10, 11, 12 or 13; or (iii) a combination thereof.

In some embodiments, the first TCRβV-binding moiety, the second TCRβV-binding moiety, or a combination thereof comprises a light chain comprising a FR1 comprising: (i) an Aspartic Acid at position 1 according to Kabat numbering: (ii) an Asparagine at position 2 according to Kabat numbering: (iii) a Leucine at position 4 according to Kabat numbering; or (iv) a combination thereof.

In some embodiments, the first TCRβV-binding moiety, the second TCRβV-binding moiety, or a combination thereof comprises a light chain comprising a FR3 comprising: (i) a Glycine at position 66 according to Kabat numbering: (ii) an Asparagine at position 69 according to Kabat numbering; (iii) a Tyrosine at position 71 according to Kabat numbering; or (iv) a combination thereof.

In some embodiments, the first TCRβV-binding moiety, the second TCRβV-binding moiety, or a combination thereof binds to an outward facing region on a TCRβV protein. In some embodiments, the outward facing region on the TCRβV protein comprises a structurally conserved region of TCRβV having a similar structure across one or more TCRβV subfamilies.

In some embodiments, the multispecific molecule includes an anti-TCRVA antibody molecule as described herein.

Described herein, in certain embodiments, is a multifunctional polypeptide molecule comprising a first polypeptide, a second polypeptide, a third polypeptide, a fourth polypeptide, and at least one cytokine polypeptide or a variant thereof, wherein the first polypeptide, the second polypeptide, the third polypeptide, and the fourth polypeptide are non-contiguous, wherein: (i) the first polypeptide comprising a first portion of a first T cell receptor variable alpha (TCRαV)-binding moiety and a first dimerization module linked to the first portion of the first TCRαV-binding moiety; (ii) the second polypeptide comprising a second portion of the first TCRαV-binding moiety; (iii) the third polypeptide comprising a first portion of a second TCRαV-binding moiety and a second dimerization module linked to the first portion of the second TCRαV-binding moiety; and (iv) the fourth polypeptide comprising a second portion of the second TCRαV-binding moiety; and wherein the at least one cytokine polypeptide or the variant thereof is covalently linked to the first polypeptide, the second polypeptide, the third polypeptide, the fourth polypeptide, or a combination thereof.

Described herein, in certain embodiments, is a multifunctional polypeptide molecule comprising a first polypeptide, a second polypeptide, a third polypeptide, and at least one cytokine polypeptide or a variant thereof, wherein the first polypeptide, the second polypeptide, and the third polypeptide are non-contiguous, wherein: (i) the first polypeptide comprising a first portion of a first TCRαV-binding moiety and a first dimerization module linked to the first portion of the first TCRαV-binding moiety; (ii) the second polypeptide comprising a second portion of the first TCRαV-binding moiety; and (iii) the third polypeptide comprising a second dimerization module; and wherein the at least one cytokine polypeptide or the variant thereof is covalently linked to the first polypeptide, the second polypeptide, the third polypeptide, or a combination thereof.

Described herein, in certain embodiments, is a multifunctional polypeptide molecule comprising a first polypeptide, a second polypeptide, a third polypeptide, and at least one cytokine polypeptide or a variant thereof, wherein the first polypeptide, the second polypeptide, and the third polypeptide are non-contiguous, wherein: (i) the first polypeptide comprising a first portion of a first TCRαV-binding moiety and a first dimerization module linked to the first portion of the first TCRαV-binding moiety; (ii) the second polypeptide comprising a second portion of the first TCRαV-binding moiety; and (iii) the third polypeptide comprising a second dimerization module; wherein the at least one cytokine polypeptide or the variant thereof is covalently linked to the first polypeptide, the second polypeptide, the third polypeptide, or a combination thereof; and wherein the multifunctional polypeptide molecule does not comprise an additional TCRαV-binding moiety except the first TCRαV-binding moiety.

In some embodiments, the first portion of the first TCRαV-binding moiety comprises a first heavy chain variable domain (VH) and a first heavy chain constant domain 1 (CH1) linked to the first VH. In some embodiments, the first CH1 is linked to the C-terminus of the first VH. In some embodiments, the second portion of the first TCRαV-binding moiety comprises a first light chain variable domain (VL) and a first light chain constant domain (CL) linked to the first VL. In some embodiments, first CL is linked to the C-terminus of the first VL. In some embodiments, wherein the first dimerization module is linked to the first portion of the first TCRαV-binding moiety. In some embodiments, the first dimerization module is linked to the C-terminus of the first portion of the first TCRαV-binding moiety. In some embodiments, wherein the first portion of the second TCRαV-binding moiety comprises a second VH and a second CH1 linked to the second VH. In some embodiments, the second CH1 is linked to the C-terminus of the second VH. In some embodiments, the second portion of the second TCRαV-binding moiety comprises a second VL and a second CL linked to the second VL. In some embodiments, the second CL is linked to the C-terminus of the second VL. In some embodiments, the second dimerization module is linked to the first portion of the second TCRαV-binding moiety. In some embodiments, the second dimerization module is linked to the C-terminus of the first portion of the second TCRαV-binding moiety.

In some embodiments, (a) the N-terminus of the first polypeptide is linked to a first cytokine polypeptide or a variant thereof; the C-terminus of the first polypeptide is linked to a second cytokine polypeptide or a variant thereof; or a combination thereof; (b) the N-terminus of the second polypeptide is linked to a third cytokine polypeptide or a variant thereof; the C-terminus of the second polypeptide is linked to a fourth cytokine polypeptide or a variant thereof; or a combination thereof; (c) the N-terminus of the third polypeptide is linked to a fifth cytokine polypeptide or a variant thereof; the C-terminus of the third polypeptide is linked to a sixth cytokine polypeptide or a variant thereof; or a combination thereof; (d) the N-terminus of the fourth polypeptide is linked to a seventh cytokine polypeptide or a variant thereof; the C-terminus of the fourth polypeptide is linked to an eighth cytokine polypeptide or a variant thereof; or a combination thereof; or (e) a combination thereof.

In some embodiments, (a-1) the N-terminus of the first polypeptide is linked to the first cytokine polypeptide or the variant thereof; the C-terminus of the first polypeptide is linked to the second cytokine polypeptide or the variant thereof; or a combination thereof; and (a-2) the N-terminus of the second polypeptide is linked to the third cytokine polypeptide or the variant thereof; the C-terminus of the second polypeptide is linked to the fourth cytokine polypeptide or the variant thereof; or a combination thereof; (b-1) the N-terminus of the first polypeptide is linked to the first cytokine polypeptide or the variant thereof; the C-terminus of the first polypeptide is linked to the second cytokine polypeptide or the variant thereof; or a combination thereof; and (b-2) the N-terminus of the third polypeptide is linked to the fifth cytokine polypeptide or the variant thereof; the C-terminus of the third polypeptide is linked to the sixth cytokine polypeptide or the variant thereof; or a combination thereof; (c-1) the N-terminus of the first polypeptide is linked to the first cytokine polypeptide or the variant thereof; the C-terminus of the first polypeptide is linked to the second cytokine polypeptide or the variant thereof; or a combination thereof; and (c-2) the N-terminus of the fourth polypeptide is linked to the seventh cytokine polypeptide or the variant thereof; the C-terminus of the fourth polypeptide is linked to the eighth cytokine polypeptide or the variant thereof; or a combination thereof; (d-1) the N-terminus of the second polypeptide is linked to the third cytokine polypeptide or the variant thereof; the C-terminus of the second polypeptide is linked to the fourth cytokine polypeptide or the variant thereof; or a combination thereof; and (d-2) the N-terminus of the third polypeptide is linked to the fifth cytokine polypeptide or the variant thereof; the C-terminus of the third polypeptide is linked to the sixth cytokine polypeptide or the variant thereof; or a combination thereof; (e-1) the N-terminus of the second polypeptide is linked to the third cytokine polypeptide or the variant thereof; the C-terminus of the second polypeptide is linked to the fourth cytokine polypeptide or the variant thereof; or a combination thereof; and (e-2) the N-terminus of the fourth polypeptide is linked to the seventh cytokine polypeptide or the variant thereof, the C-terminus of the fourth polypeptide is linked to the eighth cytokine polypeptide or the variant thereof, or a combination thereof, or (f-1) the N-terminus of the third polypeptide is linked to the fifth cytokine polypeptide or the variant thereof, the C-terminus of the third polypeptide is linked to the sixth cytokine polypeptide or the variant thereof, or a combination thereof, and (f-2) the N-terminus of the fourth polypeptide is linked to the seventh cytokine polypeptide or the variant thereof; the C-terminus of the fourth polypeptide is linked to the eighth cytokine polypeptide or the variant thereof, or a combination thereof.

In some embodiments, (a-1) the N-terminus of the first polypeptide is linked to the first cytokine polypeptide or the variant thereof, the C-terminus of the first polypeptide is linked to the second cytokine polypeptide or the variant thereof; or a combination thereof, (a-2) the N-terminus of the second polypeptide is linked to the third cytokine polypeptide or the variant thereof, the C-terminus of the second polypeptide is linked to the fourth cytokine polypeptide or the variant thereof, or a combination thereof, and (a-3) the N-terminus of the third polypeptide is linked to the fifth cytokine polypeptide or the variant thereof, the C-terminus of the third polypeptide is linked to the sixth cytokine polypeptide or the variant thereof, or a combination thereof, (b-1) the N-terminus of the first polypeptide is linked to the first cytokine polypeptide or the variant thereof, the C-terminus of the first polypeptide is linked to the second cytokine polypeptide or the variant thereof; or a combination thereof; (b-2) the N-terminus of the second polypeptide is linked to the third cytokine polypeptide or the variant thereof; the C-terminus of the second polypeptide is linked to the fourth cytokine polypeptide or the variant thereof, or a combination thereof; and (b-3) the N-terminus of the fourth polypeptide is linked to the seventh cytokine polypeptide or the variant thereof, the C-terminus of the fourth polypeptide is linked to the eighth cytokine polypeptide or the variant thereof, or a combination thereof, or (c-1) the N-terminus of the second polypeptide is linked to the third cytokine polypeptide or the variant thereof, the C-terminus of the second polypeptide is linked to the fourth cytokine polypeptide or the variant thereof, or a combination thereof, (c-2) the N-terminus of the third polypeptide is linked to the fifth cytokine polypeptide or the variant thereof, the C-terminus of the third polypeptide is linked to the sixth cytokine polypeptide or the variant thereof, or a combination thereof, and (c-3) the N-terminus of the fourth polypeptide is linked to the seventh cytokine polypeptide or the variant thereof; the C-terminus of the fourth polypeptide is linked to the eighth cytokine polypeptide or the variant thereof, or a combination thereof.

In some embodiments, (1) the N-terminus of the first polypeptide is linked to the first cytokine polypeptide or the variant thereof, the C-terminus of the first polypeptide is linked to the second cytokine polypeptide or the variant thereof, or a combination thereof, (2) the N-terminus of the second polypeptide is linked to the third cytokine polypeptide or the variant thereof; the C-terminus of the second polypeptide is linked to the fourth cytokine polypeptide or the variant thereof, or a combination thereof, (3) the N-terminus of the third polypeptide is linked to the fifth cytokine polypeptide or the variant thereof; the C-terminus of the third polypeptide is linked to the sixth cytokine polypeptide or the variant thereof, or a combination thereof, and (4) the N-terminus of the fourth polypeptide is linked to the seventh cytokine polypeptide or the variant thereof; the C-terminus of the fourth polypeptide is linked to the eighth cytokine polypeptide or the variant thereof; or a combination thereof.

In some embodiments, the first cytokine polypeptide, the second cytokine polypeptide, or a combination thereof is within a single contiguous polypeptide chain of the first polypeptide, the third cytokine polypeptide, the fourth cytokine polypeptide, or a combination thereof is within a single contiguous polypeptide chain of the second polypeptide, the fifth cytokine polypeptide, the sixth cytokine polypeptide, or a combination thereof is within a single contiguous polypeptide chain of the third polypeptide, the seventh cytokine polypeptide, the eighth cytokine polypeptide, or a combination thereof is within a single contiguous polypeptide chain of the fourth polypeptide, or a combination thereof.

In some embodiments, (a) the N-terminus of the first polypeptide is linked to a first cytokine polypeptide or a variant thereof; the C-terminus of the first polypeptide is linked to a second cytokine polypeptide or a variant thereof; or a combination thereof; (b) the N-terminus of the second polypeptide is linked to a third cytokine polypeptide or a variant thereof; the C-terminus of the second polypeptide is linked to a fourth cytokine polypeptide or a variant thereof; or a combination thereof; (c) the N-terminus of the third polypeptide is linked to a fifth cytokine polypeptide or a variant thereof; the C-terminus of the third polypeptide is linked to a sixth cytokine polypeptide or a variant thereof; or a combination thereof; or (d) a combination thereof.

In some embodiments, (a-1) the N-terminus of the first polypeptide is linked to the first cytokine polypeptide or the variant thereof; the C-terminus of the first polypeptide is linked to the second cytokine polypeptide or the variant thereof; or a combination thereof; and (a-2) the N-terminus of the second polypeptide is linked to the third cytokine polypeptide or the variant thereof; the C-terminus of the second polypeptide is linked to the fourth cytokine polypeptide or the variant thereof; or a combination thereof; (b-1) the N-terminus of the first polypeptide is linked to the first cytokine polypeptide or the variant thereof; the C-terminus of the first polypeptide is linked to the second cytokine polypeptide or the variant thereof; or a combination thereof; and (b-2) the N-terminus of the third polypeptide is linked to the fifth cytokine polypeptide or the variant thereof; the C-terminus of the third polypeptide is linked to the sixth cytokine polypeptide or the variant thereof; or a combination thereof; or (c-1) the N-terminus of the second polypeptide is linked to the third cytokine polypeptide or the variant thereof; the C-terminus of the second polypeptide is linked to the fourth cytokine polypeptide or the variant thereof; or a combination thereof; and (c-2) the N-terminus of the third polypeptide is linked to the fifth cytokine polypeptide or the variant thereof; the C-terminus of the third polypeptide is linked to the sixth cytokine polypeptide or the variant thereof; or a combination thereof.

In some embodiments, (1) the N-terminus of the first polypeptide is linked to the first cytokine polypeptide or the variant thereof; the C-terminus of the first polypeptide is linked to the second cytokine polypeptide or the variant thereof; or a combination thereof; (2) the N-terminus of the second polypeptide is linked to the third cytokine polypeptide or the variant thereof; the C-terminus of the second polypeptide is linked to the fourth cytokine polypeptide or the variant thereof; or a combination thereof; and (3) the N-terminus of the third polypeptide is linked to the fifth cytokine polypeptide or the variant thereof, the C-terminus of the third polypeptide is linked to the sixth cytokine polypeptide or the variant thereof; or a combination thereof.

In some embodiments, the first cytokine polypeptide, the second cytokine polypeptide, or a combination thereof is within a single contiguous polypeptide chain of the first polypeptide, the third cytokine polypeptide, the fourth cytokine polypeptide, or a combination thereof is within a single contiguous polypeptide chain of the second polypeptide, the fifth cytokine polypeptide, the sixth cytokine polypeptide, or a combination thereof is within a single contiguous polypeptide chain of the third polypeptide, or a combination thereof.

In some embodiments, the multifunctional polypeptide molecule as described herein further comprises a linker between the first portion of the first TCRαV-binding moiety and the first dimerization module, a linker between the first portion of the second TCRαV-binding moiety and the second dimerization module, a linker between the first VH and the first CH1, a linker between the first VL and the first CL, a linker between the second VH and the second CH1, a linker between the second VL and the second CL, a linker between the at least one cytokine polypeptide or the variant thereof and the first polypeptide, a linker between the at least one cytokine polypeptide or the variant thereof and the second polypeptide, a linker between the at least one cytokine polypeptide or the variant thereof and the third polypeptide, a linker between the at least one cytokine polypeptide or the variant thereof and the fourth polypeptide, or a combination thereof.

In some embodiments, the multifunctional polypeptide molecule as described herein further comprises comprising a linker between the first portion of the first TCRαV-binding moiety and the first dimerization module, a linker between the first VH and the first CH1, a linker between the first VL and the first CL, a linker between the at least one cytokine polypeptide or the variant thereof and the first polypeptide, a linker between the at least one cytokine polypeptide or the variant thereof and the second polypeptide, a linker between the at least one cytokine polypeptide or the variant thereof and the third polypeptide, or a combination thereof. In some embodiments, linker is selected from the group consisting of a cleavable linker, a non-cleavable linker, a peptide linker, a flexible linker, a rigid linker, a helical linker, and a non-helical linker. In some embodiments, the linker is the peptide linker and wherein the linker is a GS linker. In some embodiments, the linker is the peptide linker and wherein the linker comprises the sequence of SEQ ID NO: 3308 or SEQ ID NO: 3643.

Described herein, in certain embodiments, is a multifunctional polypeptide molecule comprising a first polypeptide, a second polypeptide, a third polypeptide, a fourth polypeptide, a first cytokine polypeptide or a variant thereof, and a second cytokine polypeptide or a variant thereof, wherein the first polypeptide, the second polypeptide, the third polypeptide, and the fourth polypeptide are non-contiguous, wherein: (i) the first polypeptide comprising a first portion of a first TCRαV-binding moiety and a first dimerization module linked to the first portion of the first TCRαV-binding moiety; (ii) the second polypeptide comprising a second portion of the first TCRαV-binding moiety; (iii) the third polypeptide comprising a first portion of a second TCRαV-binding moiety and a second dimerization module linked to the first portion of the second TCRαV-binding moiety; and (iv) the fourth polypeptide comprising a second portion of the second TCRαV-binding moiety, and wherein the first cytokine polypeptide or the variant thereof is covalently linked to the C-terminus of the second polypeptide, and the second cytokine polypeptide or the variant thereof is covalently linked to the C-terminus of the fourth polypeptide.

Described herein, in certain embodiments, is a multifunctional polypeptide molecule comprising a first polypeptide, a second polypeptide, a third polypeptide, a fourth polypeptide, a cytokine polypeptide or a variant thereof, wherein the first polypeptide, the second polypeptide, the third polypeptide, and the fourth polypeptide are non-contiguous, wherein: (i) the first polypeptide comprising a first portion of a first TCRαV-binding moiety and a first dimerization module linked to the first portion of the first TCRαV-binding moiety; (ii) the second polypeptide comprising a second portion of the first TCRαV-binding moiety; (iii) the third polypeptide comprising a first portion of a second TCRαV-binding moiety and a second dimerization module linked to the first portion of the second TCRαV-binding moiety; and (iv) the fourth polypeptide comprising a second portion of the second TCRαV-binding moiety; and wherein the cytokine polypeptide or the variant thereof is covalently linked to the C-terminus of the second polypeptide or the C-terminus of the fourth polypeptide.

Described herein, in certain embodiments, is a multifunctional polypeptide molecule comprising a first polypeptide, a second polypeptide, a third polypeptide, a fourth polypeptide, a cytokine polypeptide or a variant thereof, wherein the first polypeptide, the second polypeptide, the third polypeptide, and the fourth polypeptide are non-contiguous, wherein: (i) the first polypeptide comprising a first portion of a first TCRαV-binding moiety and a first dimerization module linked to the first portion of the first TCRαV-binding moiety; (ii) the second polypeptide comprising a second portion of the first TCRαV-binding moiety; (iii) the third polypeptide comprising a first portion of a second TCRαV-binding moiety and a second dimerization module linked to the first portion of the second TCRαV-binding moiety; and (iv) the fourth polypeptide comprising a second portion of the second TCRαV-binding moiety; and wherein the cytokine polypeptide or the variant thereof is covalently linked to the C-terminus of the first polypeptide or the C-terminus of the third polypeptide.

Described herein, in certain embodiments, is a multifunctional polypeptide molecule comprising a first polypeptide, a second polypeptide, a third polypeptide, and a cytokine polypeptide or a variant thereof, wherein the first polypeptide, the second polypeptide, and the third polypeptide are non-contiguous, wherein: (i) the first polypeptide comprising a first portion of a first TCRαV-binding moiety and a first dimerization module linked to the first portion of the first TCRαV-binding moiety; (ii) the second polypeptide comprising a second portion of the first TCRαV-binding moiety; and (iii) the third polypeptide comprising a second dimerization module; wherein the at least one cytokine polypeptide or the variant thereof is covalently linked to the N terminus of the third polypeptide; and wherein the multifunctional polypeptide molecule does not comprise an additional TCRαV-binding moiety except the first TCRαV-binding moiety.

In some embodiments, the first portion of the first TCRαV-binding moiety comprises a first VH and a first CH1 linked to the first VH. In some embodiments, the first CH1 is linked to the C-terminus of the first VH.

In some embodiments, the second portion of the first TCRαV-binding moiety comprises a first VL and a first CL linked to the first VL. In some embodiments, first CL is linked to the C-terminus of the first VL.

In some embodiments, the first dimerization module is linked to the first portion of the first TCRαV-binding moiety. In some embodiments, the first dimerization module is linked to the C-terminus of the first portion of the first TCRαV-binding moiety. In some embodiments, the first portion of the second TCRαV-binding moiety comprises a second VH and a second CH1 linked to the second VH. In some embodiments, the second CH1 is linked to the C-terminus of the second VH. In some embodiments, the second portion of the second TCRαV-binding moiety comprises a second VL and a second CL linked to the second VL. In some embodiments, the second CL is linked to the C-terminus of the second VL. In some embodiments, the second dimerization module is linked to the first portion of the second TCRαV-binding moiety. In some embodiments, the second dimerization module is linked to the C-terminus of the first portion of the second TCRαV-binding moiety.

In some embodiments, the multifunctional polypeptide molecule as described herein further comprises a linker between the first portion of the first TCRαV-binding moiety and the first dimerization module, a linker between the first portion of the second TCRαV-binding moiety and the second dimerization module, a linker between the first VH and the first CH1, a linker between the first VL and the first CL, a linker between the second VH and the second CH1, a linker between the second VL and the second CL, a linker between the at least one cytokine polypeptide or the variant thereof and the first polypeptide, a linker between the at least one cytokine polypeptide or the variant thereof and the second polypeptide, a linker between the at least one cytokine polypeptide or the variant thereof and the third polypeptide, a linker between the at least one cytokine polypeptide or the variant thereof and the fourth polypeptide, or a combination thereof. In some embodiments, the multifunctional polypeptide molecule as described herein further comprises a linker between the first portion of the first TCRαV-binding moiety and the first dimerization module, a linker between the first VH and the first CH1, a linker between the first VL and the first CL, a linker between the at least one cytokine polypeptide or the variant thereof and the third polypeptide, or a combination thereof. In some embodiments, linker is selected from the group consisting of a cleavable linker, a non-cleavable linker, a peptide linker, a flexible linker, a rigid linker, a helical linker, and a non-helical linker. In some embodiments, the linker is the peptide linker and wherein the linker is a GS linker. In some embodiments, the linker is the peptide linker and wherein the linker comprises the sequence of SEQ ID NO: 3308 or SEQ ID NO: 3643.

In some embodiments, the first TCRαV-binding moiety, the second TCRαV-binding moiety, or a combination thereof comprises any one selected from the group consisting of a Fab, F(ab′)2, Fv, a single chain Fv (scFv), a single domain antibody, a diabody (dAb), a camelid antibody and a combination thereof. In some embodiments, the first TCRαV-binding moiety, the second TCRαV-binding moiety, or a combination thereof comprises a scFv or a Fab.

In some embodiments, the multifunctional polypeptide molecule does not comprise an additional antigen-binding moiety except the TCRαV-binding moiety. In some embodiments, the multifunctional polypeptide molecule further comprise an additional antigen-binding moiety that is not the TCRαV-binding moiety.

Described herein, in certain embodiments, is a multifunctional polypeptide molecule comprising a first polypeptide, a second polypeptide, and at least one cytokine polypeptide or a variant thereof, wherein the first polypeptide and the second polypeptide are non-contiguous, wherein: (i) the first polypeptide comprising a first TCRαV-binding moiety and a first dimerization module linked to the C-terminus of the first TCRαV-binding moiety, wherein the first TCRαV-binding moiety comprises a first VL and a first VH; and (ii) the second polypeptide comprising a second TCRαV-binding moiety and a second dimerization module linked to the C-terminus of the second TCRαV-binding moiety; wherein the at least one cytokine polypeptide or the variant thereof is covalently linked to the first polypeptide, the second polypeptide, or a combination thereof; wherein the first TCRαV-binding moiety, the second TCRαV-binding moiety, or a combination thereof comprises a scFv; and wherein the multifunctional polypeptide molecule does not comprise an additional antigen-binding moiety except the first TCRαV-binding moiety and the second TCRαV-binding moiety.

Described herein, in certain embodiments, is a multifunctional polypeptide molecule comprising a first polypeptide, a second polypeptide, and at least one cytokine polypeptide or a variant thereof, wherein the first polypeptide and the second polypeptide are non-contiguous, wherein: (i) the first polypeptide comprising a first TCRαV-binding moiety and a first dimerization module linked to the C-terminus of the first TCRαV-binding moiety, wherein the first TCRαV-binding moiety comprises a first VL and a first VH; and (ii) the second polypeptide comprising a second dimerization module; wherein the at least one cytokine polypeptide or the variant thereof is covalently linked to the first polypeptide, the second polypeptide, or a combination thereof; wherein the first TCRαV-binding moiety comprises a scFv; wherein the multifunctional polypeptide molecule does not comprise an additional antigen-binding moiety except the first TCRαV-binding moiety, and wherein the multifunctional polypeptide molecule does not comprise an additional TCRαV-binding moiety except the first TCRαV-binding moiety.

In some embodiments, (a) the N-terminus of the first polypeptide is linked to a first cytokine polypeptide or a variant thereof; the C-terminus of the first polypeptide is linked to a second cytokine polypeptide or a variant thereof; or a combination thereof; (b) the N-terminus of the second polypeptide is linked to a third cytokine polypeptide or a variant thereof; the C-terminus of the second polypeptide is linked to a fourth cytokine polypeptide or a variant thereof; or a combination thereof; or (e) a combination thereof.

In some embodiments, the first cytokine polypeptide, the second cytokine polypeptide, or a combination thereof is within a single contiguous polypeptide chain of the first polypeptide, the third cytokine polypeptide, the fourth cytokine polypeptide, or a combination thereof is within a single contiguous polypeptide chain of the second polypeptide, or a combination thereof.

In some embodiments, the multifunctional polypeptide molecule as described herein further comprises a linker between the first TCRαV-binding moiety and the first dimerization module, a linker between the second TCRαV-binding moiety and the second dimerization module, a linker between the at least one cytokine polypeptide or the variant thereof and the first polypeptide, a linker between the at least one cytokine polypeptide or the variant thereof and the second polypeptide, or a combination thereof.

In some embodiments, the multifunctional polypeptide molecule as described herein further comprises a linker between the first TCRαV-binding moiety and the first dimerization module, a linker between the at least one cytokine polypeptide or the variant thereof and the first polypeptide, a linker between the at least one cytokine polypeptide or the variant thereof and the second polypeptide, or a combination thereof. In some embodiments, the linker is selected from the group consisting of a cleavable linker, a non-cleavable linker, a peptide linker, a flexible linker, a rigid linker, a helical linker, and a non-helical linker. In some embodiments, the linker is the peptide linker and wherein the linker is a GS linker. In some embodiments, the linker is the peptide linker and wherein the linker comprises the sequence of SEQ ID NO: 3308 or SEQ ID NO: 3643.

In some embodiments, the multifunctional polypeptide molecule comprises at least two of the cytokine polypeptides. In some embodiments, the multifunctional polypeptide molecule comprises at least three of the cytokine polypeptides. In some embodiments, the multifunctional polypeptide molecule comprises at least four of the cytokine polypeptides. In some embodiments, the multifunctional polypeptide molecule comprises at least five of the cytokine polypeptides. In some embodiments, the multifunctional polypeptide molecule comprises at least six of the cytokine polypeptides. In some embodiments, the multifunctional polypeptide molecule comprises at least seven of the cytokine polypeptides. In some embodiments, the multifunctional polypeptide molecule comprises at least eight of the cytokine polypeptides. In some embodiments, the multifunctional polypeptide molecule comprises two of the cytokine polypeptides. In some embodiments, the multifunctional polypeptide molecule comprises three of the cytokine polypeptides. In some embodiments, the multifunctional polypeptide molecule comprises four of the cytokine polypeptides. In some embodiments, the multifunctional polypeptide molecule comprises five of the cytokine polypeptides. In some embodiments, the multifunctional polypeptide molecule comprises six of the cytokine polypeptides. In some embodiments, the multifunctional polypeptide molecule comprises seven of the cytokine polypeptides. In some embodiments, the multifunctional polypeptide molecule comprises eight of the cytokine polypeptides. In some embodiments, the multifunctional polypeptide molecule comprises two of the cytokine polypeptide, each of which is linked to the first polypeptide and the second polypeptide; the first polypeptide and the third polypeptides; the first polypeptide and the fourth polypeptide; the second and the third polypeptide; the second polypeptide and the fourth polypeptide; or the third polypeptide and the fourth polypeptide, respectively. In some embodiments, the multifunctional polypeptide molecule comprises three of the cytokine polypeptides, each of which is linked to the first polypeptide, the second polypeptide, and the third polypeptide; the first polypeptide, the second polypeptide, and the fourth polypeptide; the first polypeptide, the third polypeptide, and the fourth polypeptide; or the second polypeptide, the third polypeptide, and the fourth polypeptide, respectively. In some embodiments, the multifunctional polypeptide molecule comprises four of the cytokine polypeptides, each of which is linked to the first polypeptide, the second polypeptide, the third polypeptide, and the fourth polypeptide, respectively. In some embodiments, the cytokine polypeptide is not linked to the polypeptides that comprise the first TCRαV-binding moiety.

In some embodiments, the at least one cytokine polypeptide is selected from the group consisting of interleukin-2 (IL-2) or a fragment or a variant thereof, interleukin-7 (IL-7) or a fragment or a variant thereof, interleukin-12 (IL-12) or a fragment or a variant thereof, interleukin-15 (IL-15) or a fragment or a variant thereof, interleukin-18 (IL-18) or a fragment or a variant thereof, interleukin-21 (IL-21) or a fragment or a variant thereof, or interferon gamma or a fragment or a variant thereof, or a combination thereof.

In some embodiments, the at least one cytokine polypeptide comprises interleukin-2 (IL-2) or a fragment thereof. In some embodiments, the at least one cytokine polypeptide is interleukin-2 (IL-2) or a fragment thereof. In some embodiments, the at least one cytokine polypeptide comprises a sequence having at least 75%, 80%, 85%, 90%, 95%, 99%, 99.5%, 99.9%, or 100% sequence identity to the sequence of SEQ ID NO: 2191. In some embodiments, the at least one cytokine polypeptide comprises the sequence of SEQ ID NO: 2191. In some embodiments, the sequence of the at least one cytokine polypeptide is a sequence having at least 75%, 80%, 85%, 90%, 95%, 99%, 99.5%, 99.9%, or 100% sequence identity to the sequence of SEQ ID NO: 2191. In some embodiments, the sequence of the at least one cytokine polypeptide is the sequence of SEQ ID NO: 2191.

In some embodiments, the variant of the at least one cytokine polypeptide comprises an IL-2 variant comprising a mutation. In some embodiments, the mutation comprises an insertion mutation, a deletion mutation, or a substitution mutation. In some embodiments, the mutation comprises the substitution mutation. In some embodiments, the variant comprises an IL-2 variant comprising C125A mutation. In some embodiments, the variant of the at least one cytokine polypeptide is an IL-2 variant comprising a mutation. In some embodiments, the mutation is an insertion mutation, a deletion mutation, or a substitution mutation. In some embodiments, the mutation is the substitution mutation. In some embodiments, the variant is an IL-2 variant comprising C125A mutation. In some embodiments, the variant comprises a sequence having at least 75%, 80%, 85%, 90%, 95%, 99%, 99.5%, 99.9%, or 100% sequence identity to the sequence of SEQ ID NO: 2270. In some embodiments, the variant comprises the sequence of SEQ ID NO: 2270. In some embodiments, the sequence of the variant is a sequence having at least 75%, 80%, 85%, 90%, 95%, 99%, 99.5%, 99.9%, or 100/6 sequence identity to the sequence of SEQ ID NO: 2270. In some embodiments, the sequence of the variant is the sequence of SEQ ID NO: 2270.

In some embodiments, the first dimerization module comprises a first immunoglobulin constant regions (Fc regions) and the second dimerization module comprises a second Fc region. In some embodiments, the first dimerization module is a first immunoglobulin constant regions (Fc regions) and the second dimerization module is a second Fc region.

In some embodiments, the first Fc region, the second Fc region, or a combination thereof is selected from an IgG1 Fc region or a fragment thereof, an IgG2 Fc region or a fragment thereof, an IgG3 Fc region or a fragment thereof, an IgGA1 Fc region or a fragment thereof, an IgGA2 Fc region or a fragment thereof, an IgG4 Fc region or a fragment thereof, an IgJ Fc region or a fragment thereof, an IgM Fc region or a fragment thereof, an IgD Fc region or a fragment thereof, and an IgE Fc region or a fragment thereof.

In some embodiments, the first Fc region, the second Fc region, or a combination thereof is selected from a human IgG1 Fc region or a fragment thereof, a human IgG2 Fc region or a fragment thereof, and a human IgG4 Fc region or a fragment thereof.

In some embodiments, the first Fc region, the second Fc region, or a combination thereof comprises an Fc interface with one or more of: a paired cavity-protuberance, an electrostatic interaction, or a strand-exchange, wherein the dimerization of the first Fc region and the second Fc region is enhanced as indicated by a greater ratio of heteromultimer:homomultimer forms relative to a dimerization of Fc regions with a non-engineered interface. In some embodiments, the dimerization of the first Fc region and the second Fc region is enhanced at least by 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 150 fold, 200 fold, 250 fold, 300 fold, 250 fold, 400 fold, 450 fold, 500 fold, 550 fold, 600 fold, 650 fold, 700 fold, 750 fold, 800 fold, 850 fold, 900 fold, 950 fold, 1000 fold, 2000 fold, 3000 fold, 4000 fold, 5000 fold, 6000 fold, 7000 fold, 8000 fold, 9000 fold, or 10000 fold relative to a dimerization of Fc regions with a non-engineered interface. In some embodiments, the dimerization of the first Fc region and the second Fc region is enhanced at most by 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 150 fold, 200 fold, 250 fold, 300 fold, 250 fold, 400 fold, 450 fold, 500 fold, 550 fold, 600 fold, 650 fold, 700 fold, 750 fold, 800 fold, 850 fold, 900 fold, 950 fold, 1000 fold, 2000 fold, 3000 fold, 4000 fold, 5000 fold, 6000 fold, 7000 fold, 8000 fold, 9000 fold, or 10000 fold relative to a dimerization of Fc regions with a non-engineered interface. In some embodiments, the dimerization of the first Fc region and the second Fc region is enhanced by 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 150 fold, 200 fold, 250 fold, 300 fold, 250 fold, 400 fold, 450 fold, 500 fold, 550 fold, 600 fold, 650 fold, 700 fold, 750 fold, 800 fold, 850 fold, 900 fold, 950 fold, 1000 fold, 2000 fold, 3000 fold, 4000 fold, 5000 fold, 6000 fold, 7000 fold, 8000 fold, 9000 fold, or 10000 fold relative to a dimerization of Fc regions with a non-engineered interface.

In some embodiments, the first Fc region, the second Fc region, or a combination thereof comprises an amino acid substitution listed in Table 14.

In some embodiments, the first Fc region, the second Fc region, or a combination thereof comprises an Asn297Ala (N297A) mutation or a Leu234Ala/Leu235Ala (LALA) mutation.

In some embodiments, the first Fc region, the second Fc region, or a combination thereof comprises a sequence having at least 75%, 80%, 85%, 90%, 95%, 99%, 99.5%, 99.9%, or 100% sequence identity to the sequence of SEQ ID NO: 40, SEQ ID NO: 42, SEQ ID NO: 3645, SEQ ID NO: 3646, SEQ ID NO: 3647, SEQ ID NO:3648, or SEQ ID NO: 3649. In some embodiments, the first Fc region, the second Fc region, or a combination thereof comprises the sequence of SEQ ID NO: 40, SEQ ID NO: 42, SEQ ID NO: 3645, SEQ ID NO: 3646, SEQ ID NO: 3647, SEQ ID NO:3648, or SEQ ID NO: 3649.

In some embodiments, the sequence of the first Fc region, the second Fc region, or a combination thereof is a sequence having at least 75%, 80%, 85%, 90%, 95%, 99%, 99.5%, 99.9%, or 100% sequence identity to the sequence of SEQ ID NO: 40, SEQ ID NO: 42, SEQ ID NO: 3645, SEQ ID NO: 3646, SEQ ID NO: 3647, SEQ ID NO:3648, or SEQ ID NO: 3649. In some embodiments, the sequence of the first Fc region, the second Fc region, or a combination thereof is the sequence of SEQ ID NO: 40, SEQ ID NO: 42, SEQ ID NO: 3645, SEQ ID NO: 3646, SEQ ID NO: 3647, SEQ ID NO:3648, or SEQ ID NO: 3649.

In some embodiments, the first TCRαV-binding moiety, the second TCRαV-binding moiety, or a combination thereof binds to one or more of a TCRαV subfamily selected from the group consisting of: (i) TCRα V12 subfamily comprising one or more selected from TCRα V12-1*01, TCRα V12-1*02, TCRα V12-2*01, TCRα V12-2*02, TCRα V12-2*03, TCRα V12-3*01, and TCRα V12-3*02; (ii) TCRα V13 subfamily comprising one or more selected from TCRα V13-1*01, TCRα V13-1*02, TCRα V13-1*03, TCRα V13-2*01, and TCRα V13-2*02; (iii) TCRα V19 subfamily; (iv) TCRα V21 subfamily comprising one or more selected from TCRα V21-1*01 and TCRα V21-1*02; and (v) TCRα V30 subfamily comprising one or more selected from TCRα V30-1*01, TCRα V30-1*02, TCRα V30-1*03, TCRα V30-1*04, and TCRα V30-1*05.

In some embodiments, the first TCRαV-binding moiety and the second TCRαV-binding moiety are same. In some embodiments, the first TCRαV-binding moiety and the second TCRαV-binding moiety are different.

In some embodiments, the first TCRαV-binding moiety, the second TCRαV-binding moiety, or a combination thereof comprises: (i) a VH comprising a framework region (FR) comprising a framework 1 (FR1), a framework region 2 (FR2), a framework region 3 (FR3), and a framework region 4 (FR4) that have at least 75%, 80%, 85%, 90%, 95%, 99%, 99.5%, 99.9%, or 100% sequence identity with a non-murine germline FR1, a non-murine germline FR2, a non-murine germline FR3, and a non-murine germline FR4; (ii) a VL comprising a FR comprising a FR1, a FR2, a FR3, and a FR4 that have at least 75%, 80%, 85%, 90%, 95%, 99%, 99.5%, 99.9%, or 100% sequence identity with a non-murine germline FR1, a non-murine germline FR2, a non-murine germline FR3, and a non-murine germline FR4; or (iii) a combination thereof. In some embodiments, the first TCRαV-binding moiety, the second TCRαV-binding moiety, or a combination thereof comprises: (i) a VH comprising a FR comprising a FR1, a FR2, a FR3, and a FR4 having the sequences of a non-murine germline FR1, a non-murine germline FR2, a non-murine germline FR3, and a non-murine germline FR4; (ii) a VL comprising a FR comprising a FR1, a FR2, a FR3, and a FR4 having the sequences of a non-murine germline FR1, a non-murine germline FR2, a non-murine germline FR3, and a non-murine germline FR4; or (iii) a combination thereof.

In some embodiments, the first TCRαV-binding moiety, the second TCRαV-binding moiety, or a combination thereof comprises: (i) a VH comprising a FR1, a FR2, a FR3, and a FR4 that have at least 75%, 80%, 85%, 90%, 95%, 99%, 99.5%, 99.9%, or 100% sequence identity with a non-murine germline FR1, a non-murine germline FR2, a non-murine germline FR3, and a non-murine germline FR4, respectively; (ii) a VL comprising a FR comprising a FR1, a FR2, a FR3, and a FR4 that have at least 75%, 80%, 85%, 90%, 95%, 99%, 99.5%, 99.9%, or 100% sequence identity with a non-murine germline FR1, a non-murine germline FR2, a non-murine germline FR3, and a non-murine germline FR4, respectively; or (iii) a combination thereof. In some embodiments, the first TCRαV-binding moiety, the second TCRαV-binding moiety, or a combination thereof comprises: (i) a VH comprising a FR comprising a FR1, a FR2, a FR3, and a FR4 having the sequences of a non-murine germline FR1, a non-murine germline FR2, a non-murine germline FR3, and a non-murine germline FR4, respectively; (ii) a VL comprising a FR comprising a FR1, a FR2, a FR3, and a FR4 having the sequences of a non-murine germline FR1, a non-murine germline FR2, a non-murine germline FR3, and a non-murine germline FR4, respectively; or (iii) a combination thereof.

In some embodiments, the first polypeptide, the second polypeptide, the third polypeptide, the fourth polypeptide, or a combination thereof comprises a heavy chain constant region having a sequence having at least 75%, 80%, 85%, 90%, 95%, 99%, 99.5%, 99.9%, or 100% sequence identity to any one of the sequences listed in Table 1 or a combination thereof. In some embodiments, the first polypeptide, the second polypeptide, the third polypeptide, the fourth polypeptide, or a combination thereof comprises a heavy chain constant region having any one of the sequences listed in Table 1 or a combination thereof. In some embodiments, the first polypeptide, the second polypeptide, the third polypeptide, the fourth polypeptide, or a combination thereof comprises a heavy chain constant region of which sequence is a sequence having at least 75%, 80%, 85%, 90%, 95%, 99%, 99.5%, 99.9%, or 100% sequence identity to any one of the sequences listed in Table 1 or a combination thereof. In some embodiments, the first polypeptide, the second polypeptide, the third polypeptide, the fourth polypeptide, or a combination thereof comprises a heavy chain constant region having any one of the heavy chain constant region sequences listed in Table 1 or a combination thereof. In some embodiments, the first polypeptide, the second polypeptide, the third polypeptide, the fourth polypeptide, or a combination thereof comprises a heavy chain constant region of an IgM or a fragment thereof. In some embodiments, the heavy chain constant region of the IgM comprises a sequence having at least 75%, 80%, 85%, 90%, 95%, 99%, 99.5%, 99.9%, or 100% sequence identity to the sequence of SEQ ID NO: 73. In some embodiments, the heavy chain constant region of the IgM comprises the sequence of SEQ ID NO: 73. In some embodiments, the sequence of the heavy chain constant region of the IgM is the sequence of SEQ ID NO: 73.

In some embodiments, the first polypeptide, the second polypeptide, the third polypeptide, the fourth polypeptide, or a combination thereof comprises a heavy chain constant region of an IgJ or a fragment thereof. In some embodiments, the heavy chain constant region of the IgJ comprises a sequence having at least 75%, 80%, 85%, 90%, 95%, 99%, 99.5%, 99.9%, or 100% sequence identity to the sequence of SEQ ID NO: 76. In some embodiments, the heavy chain constant region of the IgJ comprises the sequence of SEQ ID NO: 76. In some embodiments, the sequence of the heavy chain constant region of the IgJ is the sequence of SEQ ID NO: 76.

In some embodiments, the first polypeptide, the second polypeptide, the third polypeptide, the fourth polypeptide, or a combination thereof comprises a heavy chain constant region of an IgGA1 or a fragment thereof. In some embodiments, the heavy chain constant region of the IgGA1 comprises a sequence having at least 75%, 80%, 85%, 90%, 95%, 99%, 99.5% 99.9%, or 100% sequence identity to the sequence of SEQ ID NO: 74. In some embodiments, the heavy chain constant region of the IgGA1 comprises the sequence of SEQ ID NO: 74. In some embodiments, the sequence of the heavy chain constant region of the IgGA1 is the sequence of SEQ ID NO: 74.

In some embodiments, the first polypeptide, the second polypeptide, the third polypeptide, the fourth polypeptide, or a combination thereof comprises a heavy chain constant region of an IgGA2 or a fragment thereof. In some embodiments, the heavy chain constant region of the IgGA2 comprises a sequence having at least 75%, 80%, 85%, 90%, 95%, 99%, 99.5% 99.9%, or 100% sequence identity to the sequence of SEQ ID NO: 75. In some embodiments, the heavy chain constant region of the IgGA2 comprises the sequence of SEQ ID NO: 75. In some embodiments, the sequence of the heavy chain constant region of the IgGA2 is the sequence of SEQ ID NO: 75.

In some embodiments, the first polypeptide, the second polypeptide, the third polypeptide, the fourth polypeptide, or a combination thereof comprises a heavy chain constant region of an IgG1 or a fragment thereof. In some embodiments, the heavy chain constant region of the IgG1 comprises a sequence having at least 75%, 80%, 85%, 90%, 95%, 99%, 99.5%, 99.9%, or 100% sequence identity to the sequence of SEQ ID NO: 41. In some embodiments, the heavy chain constant region of the IgG1 comprises the sequence of SEQ ID NO: 41. In some embodiments, the sequence of the heavy chain constant region of the IgG1 is the sequence of SEQ ID NO: 41. In some embodiments, the heavy chain constant region of the IgG1 comprises a sequence having at least 75%, 80%, 85%, 90%, 95%, 99%, 99.5%, 99.9%, or 100% sequence identity to the sequence of SEQ ID NO: 3645. In some embodiments, the heavy chain constant region of the IgG1 comprises the sequence of SEQ ID NO: 3645. In some embodiments, the sequence of the heavy chain constant region of the IgG1 is the sequence of SEQ ID NO: 3645.

In some embodiments, the first polypeptide, the second polypeptide, the third polypeptide, the fourth polypeptide, or a combination thereof comprises a light chain constant region having a sequence having at least 75%, 80%, 85%, 90%, 95%, 99%, 99.5%, 99.9%, or 100% sequence identity to any one of the sequences listed in Table 1 or a combination thereof. In some embodiments, the first polypeptide, the second polypeptide, the third polypeptide, the fourth polypeptide, or a combination thereof comprises a light chain constant region having any one of the sequences listed in Table 1 or a combination thereof. In some embodiments, the first polypeptide, the second polypeptide, the third polypeptide, the fourth polypeptide, or a combination thereof comprises a light chain constant region having any one of the light chain constant region sequences listed in Table 1 or a combination thereof.

In some embodiments, the first polypeptide, the second polypeptide, the third polypeptide, the fourth polypeptide, or a combination thereof comprises a light chain constant region of a kappa chain or a fragment thereof. In some embodiments, the light chain constant region of a kappa chain comprises a light chain constant region sequence listed in Table 1.

In some embodiments, the light chain constant region of a kappa chain comprises a sequence having at least 75%, 80%, 85%, 90%, 95%, 99%, 99.5%, 99.9%, or 100% sequence identity to the sequence of SEQ ID NO: 39 or SEQ ID NO: 3644. In some embodiments, the light chain constant region of a kappa chain comprises the sequence of SEQ ID NO: 39 or SEQ ID NO: 3644. In some embodiments, the sequence of the light chain constant region of a kappa chain is a sequence having at least 75%, 80%, 85%, 90%, 95%, 99%, 99.5%, 99.9%, or 100% sequence identity to the sequence of SEQ ID NO: 39 or SEQ ID NO: 3644. In some embodiments, the sequence of the light chain constant region of a kappa chain is the sequence of SEQ ID NO: 39 or SEQ ID NO: 3644.

In some embodiments, the first TCRαV-binding moiety, the second TCRαV-binding moiety, or a combination thereof binds to an outward facing region on a TCRαV protein. In some embodiments, the outward facing region on the TCRαV protein comprises a structurally conserved region of TCRαV having a similar structure across one or more TCRαV subfamilies.

Described herein, in certain embodiments, is a multifunctional polypeptide molecule comprising a first polypeptide, a second polypeptide, a third polypeptide, a fourth polypeptide, and at least one cytokine polypeptide or a variant thereof, wherein the first polypeptide, the second polypeptide, the third polypeptide, and the fourth polypeptide are non-contiguous, wherein: (i) the first polypeptide comprising a first portion of a first T cell receptor variable alpha (TCRαV)-binding moiety and a first dimerization module linked to the first portion of the first TCRαV-binding moiety; (ii) the second polypeptide comprising a second portion of the first TCRαV-binding moiety; (iii) the third polypeptide comprising a first portion of a second TCRαV-binding moiety and a second dimerization module linked to the first portion of the second TCRαV-binding moiety; and (iv) the fourth polypeptide comprising a second portion of the second TCRαV-binding moiety; and wherein the at least one cytokine polypeptide or the variant thereof is covalently linked to the first polypeptide, the second polypeptide, the third polypeptide, the fourth polypeptide, or a combination thereof.

Described herein, in certain embodiments, is a multifunctional polypeptide molecule comprising a first polypeptide, a second polypeptide, a third polypeptide, and at least one cytokine polypeptide or a variant thereof, wherein the first polypeptide, the second polypeptide, and the third polypeptide are non-contiguous, wherein: (i) the first polypeptide comprising a first portion of a first TCRαV-binding moiety and a first dimerization module linked to the first portion of the first TCRαV-binding moiety; (ii) the second polypeptide comprising a second portion of the first TCRαV-binding moiety; and (iii) the third polypeptide comprising a second dimerization module; and wherein the at least one cytokine polypeptide or the variant thereof is covalently linked to the first polypeptide, the second polypeptide, the third polypeptide, or a combination thereof.

Described herein, in certain embodiments, is a multifunctional polypeptide molecule comprising a first polypeptide, a second polypeptide, a third polypeptide, and at least one cytokine polypeptide or a variant thereof, wherein the first polypeptide, the second polypeptide, and the third polypeptide are non-contiguous, wherein: (i) the first polypeptide comprising a first portion of a first TCRαV-binding moiety and a first dimerization module linked to the first portion of the first TCRαV-binding moiety; (ii) the second polypeptide comprising a second portion of the first TCRαV-binding moiety; and (iii) the third polypeptide comprising a second dimerization module; wherein the at least one cytokine polypeptide or the variant thereof is covalently linked to the first polypeptide, the second polypeptide, the third polypeptide, or a combination thereof; and wherein the multifunctional polypeptide molecule does not comprise an additional TCRαV-binding moiety except the first TCRαV-binding moiety.

In some embodiments, the first portion of the first TCRαV-binding moiety comprises a first heavy chain variable domain (VH) and a first heavy chain constant domain 1 (CH1) linked to the first VH. In some embodiments, the first CH1 is linked to the C-terminus of the first VH. In some embodiments, the second portion of the first TCRαV-binding moiety comprises a first light chain variable domain (VL) and a first light chain constant domain (CL) linked to the first VL. In some embodiments, first CL is linked to the C-terminus of the first VL. In some embodiments, wherein the first dimerization module is linked to the first portion of the first TCRαV-binding moiety. In some embodiments, the first dimerization module is linked to the C-terminus of the first portion of the first TCRαV-binding moiety. In some embodiments, wherein the first portion of the second TCRαV-binding moiety comprises a second VH and a second CH1 linked to the second VH. In some embodiments, the second CH1 is linked to the C-terminus of the second VH. In some embodiments, the second portion of the second TCRαV-binding moiety comprises a second VL and a second CL linked to the second VL. In some embodiments, the second CL is linked to the C-terminus of the second VL. In some embodiments, the second dimerization module is linked to the first portion of the second TCRαV-binding moiety. In some embodiments, the second dimerization module is linked to the C-terminus of the first portion of the second TCRαV-binding moiety.

In some embodiments, (a) the N-terminus of the first polypeptide is linked to a first cytokine polypeptide or a variant thereof; the C-terminus of the first polypeptide is linked to a second cytokine polypeptide or a variant thereof; or a combination thereof; (b) the N-terminus of the second polypeptide is linked to a third cytokine polypeptide or a variant thereof; the C-terminus of the second polypeptide is linked to a fourth cytokine polypeptide or a variant thereof; or a combination thereof; (c) the N-terminus of the third polypeptide is linked to a fifth cytokine polypeptide or a variant thereof; the C-terminus of the third polypeptide is linked to a sixth cytokine polypeptide or a variant thereof, or a combination thereof; (d) the N-terminus of the fourth polypeptide is linked to a seventh cytokine polypeptide or a variant thereof; the C-terminus of the fourth polypeptide is linked to an eighth cytokine polypeptide or a variant thereof; or a combination thereof; or (e) a combination thereof.

In some embodiments, (a-1) the N-terminus of the first polypeptide is linked to the first cytokine polypeptide or the variant thereof; the C-terminus of the first polypeptide is linked to the second cytokine polypeptide or the variant thereof; or a combination thereof; and (a-2) the N-terminus of the second polypeptide is linked to the third cytokine polypeptide or the variant thereof; the C-terminus of the second polypeptide is linked to the fourth cytokine polypeptide or the variant thereof; or a combination thereof; (b-1) the N-terminus of the first polypeptide is linked to the first cytokine polypeptide or the variant thereof; the C-terminus of the first polypeptide is linked to the second cytokine polypeptide or the variant thereof; or a combination thereof; and (b-2) the N-terminus of the third polypeptide is linked to the fifth cytokine polypeptide or the variant thereof; the C-terminus of the third polypeptide is linked to the sixth cytokine polypeptide or the variant thereof; or a combination thereof; (c-1) the N-terminus of the first polypeptide is linked to the first cytokine polypeptide or the variant thereof; the C-terminus of the first polypeptide is linked to the second cytokine polypeptide or the variant thereof; or a combination thereof; and (c-2) the N-terminus of the fourth polypeptide is linked to the seventh cytokine polypeptide or the variant thereof; the C-terminus of the fourth polypeptide is linked to the eighth cytokine polypeptide or the variant thereof; or a combination thereof; (d-1) the N-terminus of the second polypeptide is linked to the third cytokine polypeptide or the variant thereof; the C-terminus of the second polypeptide is linked to the fourth cytokine polypeptide or the variant thereof; or a combination thereof; and (d-2) the N-terminus of the third polypeptide is linked to the fifth cytokine polypeptide or the variant thereof; the C-terminus of the third polypeptide is linked to the sixth cytokine polypeptide or the variant thereof; or a combination thereof; (e-1) the N-terminus of the second polypeptide is linked to the third cytokine polypeptide or the variant thereof; the C-terminus of the second polypeptide is linked to the fourth cytokine polypeptide or the variant thereof; or a combination thereof; and (e-2) the N-terminus of the fourth polypeptide is linked to the seventh cytokine polypeptide or the variant thereof; the C-terminus of the fourth polypeptide is linked to the eighth cytokine polypeptide or the variant thereof; or a combination thereof; or (f-1) the N-terminus of the third polypeptide is linked to the fifth cytokine polypeptide or the variant thereof; the C-terminus of the third polypeptide is linked to the sixth cytokine polypeptide or the variant thereof; or a combination thereof; and (f-2) the N-terminus of the fourth polypeptide is linked to the seventh cytokine polypeptide or the variant thereof; the C-terminus of the fourth polypeptide is linked to the eighth cytokine polypeptide or the variant thereof; or a combination thereof.

In some embodiments, (a-1) the N-terminus of the first polypeptide is linked to the first cytokine polypeptide or the variant thereof; the C-terminus of the first polypeptide is linked to the second cytokine polypeptide or the variant thereof; or a combination thereof; (a-2) the N-terminus of the second polypeptide is linked to the third cytokine polypeptide or the variant thereof; the C-terminus of the second polypeptide is linked to the fourth cytokine polypeptide or the variant thereof; or a combination thereof; and (a-3) the N-terminus of the third polypeptide is linked to the fifth cytokine polypeptide or the variant thereof; the C-terminus of the third polypeptide is linked to the sixth cytokine polypeptide or the variant thereof; or a combination thereof; (b-1) the N-terminus of the first polypeptide is linked to the first cytokine polypeptide or the variant thereof; the C-terminus of the first polypeptide is linked to the second cytokine polypeptide or the variant thereof; or a combination thereof; (b-2) the N-terminus of the second polypeptide is linked to the third cytokine polypeptide or the variant thereof; the C-terminus of the second polypeptide is linked to the fourth cytokine polypeptide or the variant thereof; or a combination thereof; and (b-3) the N-terminus of the fourth polypeptide is linked to the seventh cytokine polypeptide or the variant thereof; the C-terminus of the fourth polypeptide is linked to the eighth cytokine polypeptide or the variant thereof; or a combination thereof; or (c-1) the N-terminus of the second polypeptide is linked to the third cytokine polypeptide or the variant thereof; the C-terminus of the second polypeptide is linked to the fourth cytokine polypeptide or the variant thereof; or a combination thereof; (c-2) the N-terminus of the third polypeptide is linked to the fifth cytokine polypeptide or the variant thereof; the C-terminus of the third polypeptide is linked to the sixth cytokine polypeptide or the variant thereof; or a combination thereof; and (c-3) the N-terminus of the fourth polypeptide is linked to the seventh cytokine polypeptide or the variant thereof; the C-terminus of the fourth polypeptide is linked to the eighth cytokine polypeptide or the variant thereof; or a combination thereof.

In some embodiments, (1) the N-terminus of the first polypeptide is linked to the first cytokine polypeptide or the variant thereof; the C-terminus of the first polypeptide is linked to the second cytokine polypeptide or the variant thereof; or a combination thereof; (2) the N-terminus of the second polypeptide is linked to the third cytokine polypeptide or the variant thereof; the C-terminus of the second polypeptide is linked to the fourth cytokine polypeptide or the variant thereof; or a combination thereof; (3) the N-terminus of the third polypeptide is linked to the fifth cytokine polypeptide or the variant thereof; the C-terminus of the third polypeptide is linked to the sixth cytokine polypeptide or the variant thereof; or a combination thereof; and (4) the N-terminus of the fourth polypeptide is linked to the seventh cytokine polypeptide or the variant thereof; the C-terminus of the fourth polypeptide is linked to the eighth cytokine polypeptide or the variant thereof; or a combination thereof.

In some embodiments, the first cytokine polypeptide, the second cytokine polypeptide, or a combination thereof is within a single contiguous polypeptide chain of the first polypeptide, the third cytokine polypeptide, the fourth cytokine polypeptide, or a combination thereof is within a single contiguous polypeptide chain of the second polypeptide, the fifth cytokine polypeptide, the sixth cytokine polypeptide, or a combination thereof is within a single contiguous polypeptide chain of the third polypeptide, the seventh cytokine polypeptide, the eighth cytokine polypeptide, or a combination thereof is within a single contiguous polypeptide chain of the fourth polypeptide, or a combination thereof.

In some embodiments, (a) the N-terminus of the first polypeptide is linked to a first cytokine polypeptide or a variant thereof; the C-terminus of the first polypeptide is linked to a second cytokine polypeptide or a variant thereof; or a combination thereof; (b) the N-terminus of the second polypeptide is linked to a third cytokine polypeptide or a variant thereof; the C-terminus of the second polypeptide is linked to a fourth cytokine polypeptide or a variant thereof; or a combination thereof; (c) the N-terminus of the third polypeptide is linked to a fifth cytokine polypeptide or a variant thereof; the C-terminus of the third polypeptide is linked to a sixth cytokine polypeptide or a variant thereof; or a combination thereof; or (d) a combination thereof.

In some embodiments, (a-1) the N-terminus of the first polypeptide is linked to the first cytokine polypeptide or the variant thereof; the C-terminus of the first polypeptide is linked to the second cytokine polypeptide or the variant thereof; or a combination thereof; and (a-2) the N-terminus of the second polypeptide is linked to the third cytokine polypeptide or the variant thereof; the C-terminus of the second polypeptide is linked to the fourth cytokine polypeptide or the variant thereof; or a combination thereof; (b-1) the N-terminus of the first polypeptide is linked to the first cytokine polypeptide or the variant thereof; the C-terminus of the first polypeptide is linked to the second cytokine polypeptide or the variant thereof; or a combination thereof; and (b-2) the N-terminus of the third polypeptide is linked to the fifth cytokine polypeptide or the variant thereof; the C-terminus of the third polypeptide is linked to the sixth cytokine polypeptide or the variant thereof; or a combination thereof; or (c-1) the N-terminus of the second polypeptide is linked to the third cytokine polypeptide or the variant thereof; the C-terminus of the second polypeptide is linked to the fourth cytokine polypeptide or the variant thereof; or a combination thereof; and (c-2) the N-terminus of the third polypeptide is linked to the fifth cytokine polypeptide or the variant thereof; the C-terminus of the third polypeptide is linked to the sixth cytokine polypeptide or the variant thereof; or a combination thereof.

In some embodiments, (1) the N-terminus of the first polypeptide is linked to the first cytokine polypeptide or the variant thereof; the C-terminus of the first polypeptide is linked to the second cytokine polypeptide or the variant thereof; or a combination thereof; (2) the N-terminus of the second polypeptide is linked to the third cytokine polypeptide or the variant thereof; the C-terminus of the second polypeptide is linked to the fourth cytokine polypeptide or the variant thereof; or a combination thereof; and (3) the N-terminus of the third polypeptide is linked to the fifth cytokine polypeptide or the variant thereof; the C-terminus of the third polypeptide is linked to the sixth cytokine polypeptide or the variant thereof; or a combination thereof.

In some embodiments, the first cytokine polypeptide, the second cytokine polypeptide, or a combination thereof is within a single contiguous polypeptide chain of the first polypeptide, the third cytokine polypeptide, the fourth cytokine polypeptide, or a combination thereof is within a single contiguous polypeptide chain of the second polypeptide, the fifth cytokine polypeptide, the sixth cytokine polypeptide, or a combination thereof is within a single contiguous polypeptide chain of the third polypeptide, or a combination thereof.

In some embodiments, the multifunctional polypeptide molecule as described herein further comprises a linker between the first portion of the first TCRαV-binding moiety and the first dimerization module, a linker between the first portion of the second TCRαV-binding moiety and the second dimerization module, a linker between the first VH and the first CH1, a linker between the first VL and the first CL, a linker between the second VH and the second CH1, a linker between the second VL and the second CL, a linker between the at least one cytokine polypeptide or the variant thereof and the first polypeptide, a linker between the at least one cytokine polypeptide or the variant thereof and the second polypeptide, a linker between the at least one cytokine polypeptide or the variant thereof and the third polypeptide, a linker between the at least one cytokine polypeptide or the variant thereof and the fourth polypeptide, or a combination thereof.

In some embodiments, the multifunctional polypeptide molecule as described herein further comprises comprising a linker between the first portion of the first TCRαV-binding moiety and the first dimerization module, a linker between the first VH and the first CH1, a linker between the first VL and the first CL, a linker between the at least one cytokine polypeptide or the variant thereof and the first polypeptide, a linker between the at least one cytokine polypeptide or the variant thereof and the second polypeptide, a linker between the at least one cytokine polypeptide or the variant thereof and the third polypeptide, or a combination thereof. In some embodiments, linker is selected from the group consisting of a cleavable linker, a non-cleavable linker, a peptide linker, a flexible linker, a rigid linker, a helical linker, and a non-helical linker. In some embodiments, the linker is the peptide linker and wherein the linker is a GS linker. In some embodiments, the linker is the peptide linker and wherein the linker comprises the sequence of SEQ ID NO: 3308 or SEQ ID NO: 3643.

Described herein, in certain embodiments, is a multifunctional polypeptide molecule comprising a first polypeptide, a second polypeptide, a third polypeptide, a fourth polypeptide, a first cytokine polypeptide or a variant thereof, and a second cytokine polypeptide or a variant thereof, wherein the first polypeptide, the second polypeptide, the third polypeptide, and the fourth polypeptide are non-contiguous, wherein: (i) the first polypeptide comprising a first portion of a first TCRαV-binding moiety and a first dimerization module linked to the first portion of the first TCRαV-binding moiety; (ii) the second polypeptide comprising a second portion of the first TCRαV-binding moiety; (iii) the third polypeptide comprising a first portion of a second TCRαV-binding moiety and a second dimerization module linked to the first portion of the second TCRαV-binding moiety; and (iv) the fourth polypeptide comprising a second portion of the second TCRαV-binding moiety; and wherein the first cytokine polypeptide or the variant thereof is covalently linked to the C-terminus of the second polypeptide, and the second cytokine polypeptide or the variant thereof is covalently linked to the C-terminus of the fourth polypeptide.

Described herein, in certain embodiments, is a multifunctional polypeptide molecule comprising a first polypeptide, a second polypeptide, a third polypeptide, a fourth polypeptide, a cytokine polypeptide or a variant thereof, wherein the first polypeptide, the second polypeptide, the third polypeptide, and the fourth polypeptide are non-contiguous, wherein: (i) the first polypeptide comprising a first portion of a first TCRαV-binding moiety and a first dimerization module linked to the first portion of the first TCRαV-binding moiety; (ii) the second polypeptide comprising a second portion of the first TCRαV-binding moiety; (iii) the third polypeptide comprising a first portion of a second TCRαV-binding moiety and a second dimerization module linked to the first portion of the second TCRαV-binding moiety, and (iv) the fourth polypeptide comprising a second portion of the second TCRαV-binding moiety; and wherein the cytokine polypeptide or the variant thereof is covalently linked to the C-terminus of the second polypeptide or the C-terminus of the fourth polypeptide.

Described herein, in certain embodiments, is a multifunctional polypeptide molecule comprising a first polypeptide, a second polypeptide, a third polypeptide, a fourth polypeptide, a cytokine polypeptide or a variant thereof, wherein the first polypeptide, the second polypeptide, the third polypeptide, and the fourth polypeptide are non-contiguous, wherein: (i) the first polypeptide comprising a first portion of a first TCRαV-binding moiety and a first dimerization module linked to the first portion of the first TCRαV-binding moiety; (ii) the second polypeptide comprising a second portion of the first TCRαV-binding moiety; (iii) the third polypeptide comprising a first portion of a second TCRαV-binding moiety and a second dimerization module linked to the first portion of the second TCRαV-binding moiety; and (iv) the fourth polypeptide comprising a second portion of the second TCRαV-binding moiety; and wherein the cytokine polypeptide or the variant thereof is covalently linked to the C-terminus of the first polypeptide or the C-terminus of the third polypeptide.

Described herein, in certain embodiments, is a multifunctional polypeptide molecule comprising a first polypeptide, a second polypeptide, a third polypeptide, and a cytokine polypeptide or a variant thereof, wherein the first polypeptide, the second polypeptide, and the third polypeptide are non-contiguous, wherein: (i) the first polypeptide comprising a first portion of a first TCRαV-binding moiety and a first dimerization module linked to the first portion of the first TCRαV-binding moiety; (ii) the second polypeptide comprising a second portion of the first TCRαV-binding moiety; and (iii) the third polypeptide comprising a second dimerization module; wherein the at least one cytokine polypeptide or the variant thereof is covalently linked to the N terminus of the third polypeptide; and wherein the multifunctional polypeptide molecule does not comprise an additional TCRαV-binding moiety except the first TCRαV-binding moiety.

In some embodiments, the first portion of the first TCRαV-binding moiety comprises a first VH and a first CH1 linked to the first VH. In some embodiments, the first CH1 is linked to the C-terminus of the first VH.

In some embodiments, the second portion of the first TCRαV-binding moiety comprises a first VL and a first CL linked to the first VL. In some embodiments, first CL is linked to the C-terminus of the first VL.

In some embodiments, the first dimerization module is linked to the first portion of the first TCRαV-binding moiety. In some embodiments, the first dimerization module is linked to the C-terminus of the first portion of the first TCRαV-binding moiety. In some embodiments, the first portion of the second TCRαV-binding moiety comprises a second VH and a second CH1 linked to the second VH. In some embodiments, the second CH1 is linked to the C-terminus of the second VH. In some embodiments, the second portion of the second TCRαV-binding moiety comprises a second VL and a second CL linked to the second VL. In some embodiments, the second CL is linked to the C-terminus of the second VL. In some embodiments, the second dimerization module is linked to the first portion of the second TCRαV-binding moiety. In some embodiments, the second dimerization module is linked to the C-terminus of the first portion of the second TCRαV-binding moiety.

In some embodiments, the multifunctional polypeptide molecule as described herein further comprises a linker between the first portion of the first TCRαV-binding moiety and the first dimerization module, a linker between the first portion of the second TCRαV-binding moiety and the second dimerization module, a linker between the first VH and the first CH1, a linker between the first VL and the first CL, a linker between the second VH and the second CH1, a linker between the second VL and the second CL, a linker between the at least one cytokine polypeptide or the variant thereof and the first polypeptide, a linker between the at least one cytokine polypeptide or the variant thereof and the second polypeptide, a linker between the at least one cytokine polypeptide or the variant thereof and the third polypeptide, a linker between the at least one cytokine polypeptide or the variant thereof and the fourth polypeptide, or a combination thereof. In some embodiments, the multifunctional polypeptide molecule as described herein further comprises a linker between the first portion of the first TCRαV-binding moiety and the first dimerization module, a linker between the first VH and the first CH1, a linker between the first VL and the first CL, a linker between the at least one cytokine polypeptide or the variant thereof and the third polypeptide, or a combination thereof. In some embodiments, linker is selected from the group consisting of a cleavable linker, a non-cleavable linker, a peptide linker, a flexible linker, a rigid linker, a helical linker, and a non-helical linker. In some embodiments, the linker is the peptide linker and wherein the linker is a GS linker. In some embodiments, the linker is the peptide linker and wherein the linker comprises the sequence of SEQ ID NO: 3308 or SEQ ID NO: 3643.

In some embodiments, the first TCRαV-binding moiety, the second TCRαV-binding moiety, or a combination thereof comprises any one selected from the group consisting of a Fab, F(ab′)2, Fv, a single chain Fv (scFv), a single domain antibody, a diabody (dAb), a camelid antibody and a combination thereof. In some embodiments, the first TCRαV-binding moiety, the second TCRαV-binding moiety, or a combination thereof comprises a scFv or a Fab.

In some embodiments, the multifunctional polypeptide molecule does not comprise an additional antigen-binding moiety except the TCRαV-binding moiety. In some embodiments, the multifunctional polypeptide molecule further comprises an additional antigen-binding moiety that is not the TCRαV-binding moiety.

Described herein, in certain embodiments, is a multifunctional polypeptide molecule comprising a first polypeptide, a second polypeptide, and at least one cytokine polypeptide or a variant thereof, wherein the first polypeptide and the second polypeptide are non-contiguous, wherein: (i) the first polypeptide comprising a first TCRαV-binding moiety and a first dimerization module linked to the C-terminus of the first TCRαV-binding moiety, wherein the first TCRαV-binding moiety comprises a first VL and a first VH; and (ii) the second polypeptide comprising a second TCRαV-binding moiety and a second dimerization module linked to the C-terminus of the second TCRαV-binding moiety; wherein the at least one cytokine polypeptide or the variant thereof is covalently linked to the first polypeptide, the second polypeptide, or a combination thereof; wherein the first TCRαV-binding moiety, the second TCRαV-binding moiety, or a combination thereof comprises a scFv; and wherein the multifunctional polypeptide molecule does not comprise an additional antigen-binding moiety except the first TCRαV-binding moiety and the second TCRαV-binding moiety.

Described herein, in certain embodiments, is a multifunctional polypeptide molecule comprising a first polypeptide, a second polypeptide, and at least one cytokine polypeptide or a variant thereof, wherein the first polypeptide and the second polypeptide are non-contiguous, wherein: (i) the first polypeptide comprising a first TCRαV-binding moiety and a first dimerization module linked to the C-terminus of the first TCRαV-binding moiety, wherein the first TCRαV-binding moiety comprises a first VL and a first VH; and (ii) the second polypeptide comprising a second dimerization module; wherein the at least one cytokine polypeptide or the variant thereof is covalently linked to the first polypeptide, the second polypeptide, or a combination thereof; wherein the first TCRαV-binding moiety comprises a scFv; wherein the multifunctional polypeptide molecule does not comprise an additional antigen-binding moiety except the first TCRαV-binding moiety; and wherein the multifunctional polypeptide molecule does not comprise an additional TCRαV-binding moiety except the first TCRαV-binding moiety.

In some embodiments, (a) the N-terminus of the first polypeptide is linked to a first cytokine polypeptide or a variant thereof, the C-terminus of the first polypeptide is linked to a second cytokine polypeptide or a variant thereof; or a combination thereof; (b) the N-terminus of the second polypeptide is linked to a third cytokine polypeptide or a variant thereof, the C-terminus of the second polypeptide is linked to a fourth cytokine polypeptide or a variant thereof, or a combination thereof; or (e) a combination thereof.

In some embodiments, the first cytokine polypeptide, the second cytokine polypeptide, or a combination thereof is within a single contiguous polypeptide chain of the first polypeptide, the third cytokine polypeptide, the fourth cytokine polypeptide, or a combination thereof is within a single contiguous polypeptide chain of the second polypeptide, or a combination thereof.

In some embodiments, the multifunctional polypeptide molecule as described herein further comprises a linker between the first TCRαV-binding moiety and the first dimerization module, a linker between the second TCRαV-binding moiety and the second dimerization module, a linker between the at least one cytokine polypeptide or the variant thereof and the first polypeptide, a linker between the at least one cytokine polypeptide or the variant thereof and the second polypeptide, or a combination thereof.

In some embodiments, the multifunctional polypeptide molecule as described herein further comprises a linker between the first TCRαV-binding moiety and the first dimerization module, a linker between the at least one cytokine polypeptide or the variant thereof and the first polypeptide, a linker between the at least one cytokine polypeptide or the variant thereof and the second polypeptide, or a combination thereof. In some embodiments, the linker is selected from the group consisting of a cleavable linker, a non-cleavable linker, a peptide linker, a flexible linker, a rigid linker, a helical linker, and a non-helical linker. In some embodiments, the linker is the peptide linker and wherein the linker is a GS linker. In some embodiments, the linker is the peptide linker and wherein the linker comprises the sequence of SEQ ID NO: 3308 or SEQ ID NO: 3643.

In some embodiments, the multifunctional polypeptide molecule comprises at least two of the cytokine polypeptides. In some embodiments, the multifunctional polypeptide molecule comprises at least three of the cytokine polypeptides. In some embodiments, the multifunctional polypeptide molecule comprises at least four of the cytokine polypeptides. In some embodiments, the multifunctional polypeptide molecule comprises at least five of the cytokine polypeptides. In some embodiments, the multifunctional polypeptide molecule comprises at least six of the cytokine polypeptides. In some embodiments, the multifunctional polypeptide molecule comprises at least seven of the cytokine polypeptides. In some embodiments, the multifunctional polypeptide molecule comprises at least eight of the cytokine polypeptides. In some embodiments, the multifunctional polypeptide molecule comprises two of the cytokine polypeptides. In some embodiments, the multifunctional polypeptide molecule comprises three of the cytokine polypeptides. In some embodiments, the multifunctional polypeptide molecule comprises four of the cytokine polypeptides. In some embodiments, the multifunctional polypeptide molecule comprises five of the cytokine polypeptides. In some embodiments, the multifunctional polypeptide molecule comprises six of the cytokine polypeptides. In some embodiments, the multifunctional polypeptide molecule comprises seven of the cytokine polypeptides. In some embodiments, the multifunctional polypeptide molecule comprises eight of the cytokine polypeptides. In some embodiments, the multifunctional polypeptide molecule comprises two of the cytokine polypeptide, each of which is linked to the first polypeptide and the second polypeptide; the first polypeptide and the third polypeptide; the first polypeptide and the fourth polypeptide; the second and the third polypeptide; the second polypeptide and the fourth polypeptide; or the third polypeptide and the fourth polypeptide, respectively. In some embodiments, the multifunctional polypeptide molecule comprises three of the cytokine polypeptide, each of which is linked to the first polypeptide, the second polypeptide, and the third polypeptide; the first polypeptide, the second polypeptide, and the fourth polypeptide; the first polypeptide, the third polypeptide, and the fourth polypeptide; or the second polypeptide, the third polypeptide, and the fourth polypeptide, respectively. In some embodiments, the multifunctional polypeptide molecule comprises four of the cytokine polypeptide, each of which is linked to the first polypeptide, the second polypeptide, the third polypeptide, and the fourth polypeptide, respectively. In some embodiments, the cytokine polypeptide is not linked to the polypeptides that comprise the first TCRαV-binding moiety.

In some embodiments, the at least one cytokine polypeptide is selected from the group consisting of interleukin-2 (IL-2) or a fragment or a variant thereof, interleukin-7 (IL-7) or a fragment or a variant thereof, interleukin-12 (IL-12) or a fragment or a variant thereof, interleukin-15 (IL-15) or a fragment or a variant thereof, interleukin-18 (IL-18) or a fragment or a variant thereof, interleukin-21 (IL-21) or a fragment or a variant thereof, or interferon gamma or a fragment or a variant thereof, or a combination thereof.

In some embodiments, the at least one cytokine polypeptide comprises interleukin-2 (IL-2) or a fragment thereof. In some embodiments, the at least one cytokine polypeptide is interleukin-2 (IL-2) or a fragment thereof. In some embodiments, the at least one cytokine polypeptide comprises a sequence having at least 75%, 80%, 85%, 90%, 95%, 99%, 99.5%, 99.9/0, or 100% sequence identity to the sequence of SEQ ID NO: 2191. In some embodiments, the at least one cytokine polypeptide comprises the sequence of SEQ ID NO: 2191. In some embodiments, the sequence of the at least one cytokine polypeptide is a sequence having at least 75%, 80%, 85%, 90%, 95%, 99%, 99.5%, 99.9%, or 100% sequence identity to the sequence of SEQ ID NO: 2191. In some embodiments, the sequence of the at least one cytokine polypeptide is the sequence of SEQ ID NO: 2191.

In some embodiments, the variant of the at least one cytokine polypeptide comprises an IL-2 variant comprising a mutation. In some embodiments, the mutation comprises an insertion mutation, a deletion mutation, or a substitution mutation. In some embodiments, the mutation comprises the substitution mutation. In some embodiments, the variant comprises an IL-2 variant comprising C125A mutation. In some embodiments, the variant of the at least one cytokine polypeptide is an IL-2 variant comprising a mutation. In some embodiments, the mutation is an insertion mutation, a deletion mutation, or a substitution mutation. In some embodiments, the mutation is the substitution mutation. In some embodiments, the variant is an IL-2 variant comprising C125A mutation. In some embodiments, the variant comprises a sequence having at least 75%, 80%, 85%, 90%, 95%, 99%, 99.5%, 99.9%, or 100% sequence identity to the sequence of SEQ ID NO: 2270. In some embodiments, the variant comprises the sequence of SEQ ID NO: 2270. In some embodiments, the sequence of the variant is a sequence having at least 75%, 80%, 85%, 90%, 95%, 99%, 99.5%, 99.9%, or 100% sequence identity to the sequence of SEQ ID NO: 2270. In some embodiments, the sequence of the variant is the sequence of SEQ ID NO: 2270.

In some embodiments, the first dimerization module comprises a first immunoglobulin constant regions (Fc regions) and the second dimerization module comprises a second Fc region. In some embodiments, the first dimerization module is a first immunoglobulin constant regions (Fc regions) and the second dimerization module is a second Fc region.

In some embodiments, the first Fc region, the second Fc region, or a combination thereof is selected from an IgG1 Fc region or a fragment thereof, an IgG2 Fc region or a fragment thereof, an IgG3 Fc region or a fragment thereof, an IgGA1 Fc region or a fragment thereof, an IgGA2 Fc region or a fragment thereof, an IgG4 Fc region or a fragment thereof, an IgJ Fc region or a fragment thereof, an IgM Fc region or a fragment thereof, an IgD Fc region or a fragment thereof, and an IgE Fc region or a fragment thereof.

In some embodiments, the first Fc region, the second Fc region, or a combination thereof is selected from a human IgG1 Fc region or a fragment thereof, a human IgG2 Fc region or a fragment thereof, and a human IgG4 Fc region or a fragment thereof.

In some embodiments, the first Fc region, the second Fc region, or a combination thereof comprises an Fc interface with one or more of: a paired cavity-protuberance, an electrostatic interaction, or a strand-exchange, wherein the dimerization of the first Fc region and the second Fc region is enhanced as indicated by a greater ratio of heteromultimer:homomultimer forms relative to a dimerization of Fc regions with a non-engineered interface. In some embodiments, the dimerization of the first Fc region and the second Fc region is enhanced at least by 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 150 fold, 200 fold, 250 fold, 300 fold, 250 fold, 400 fold, 450 fold, 500 fold, 550 fold, 600 fold, 650 fold, 700 fold, 750 fold, 800 fold, 850 fold, 900 fold, 950 fold, 1000 fold, 2000 fold, 3000 fold, 4000 fold, 5000 fold, 6000 fold, 7000 fold, 8000 fold, 9000 fold, or 10000 fold relative to a dimerization of Fc regions with a non-engineered interface. In some embodiments, the dimerization of the first Fc region and the second Fc region is enhanced at most by 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 150 fold, 200 fold, 250 fold, 300 fold, 250 fold, 400 fold, 450 fold, 500 fold, 550 fold, 600 fold, 650 fold, 700 fold, 750 fold, 800 fold, 850 fold, 900 fold, 950 fold, 1000 fold, 2000 fold, 3000 fold, 4000 fold, 5000 fold, 6000 fold, 7000 fold, 8000 fold, 9000 fold, or 10000 fold relative to a dimerization of Fc regions with a non-engineered interface. In some embodiments, the dimerization of the first Fc region and the second Fc region is enhanced by 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 150 fold, 200 fold, 250 fold, 300 fold, 250 fold, 400 fold, 450 fold, 500 fold, 550 fold, 600 fold, 650 fold, 700 fold, 750 fold, 800 fold, 850 fold, 900 fold, 950 fold, 1000 fold, 2000 fold, 3000 fold, 4000 fold, 5000 fold, 6000 fold, 7000 fold, 8000 fold, 9000 fold, or 10000 fold relative to a dimerization of Fc regions with a non-engineered interface.

In some embodiments, the first Fc region, the second Fc region, or a combination thereof comprises an amino acid substitution listed in Table 14.

In some embodiments, the first Fc region, the second Fc region, or a combination thereof comprises an Asn297Ala (N297A) mutation or a Leu234Ala/Leu235Ala (LALA) mutation.

In some embodiments, the first Fc region, the second Fc region, or a combination thereof comprises a sequence having at least 75%, 80%, 85%, 90%, 95%, 99%, 99.5%, 99.9%, or 100% sequence identity to the sequence of SEQ ID NO: 40, SEQ ID NO: 42, SEQ ID NO: 3645, SEQ ID NO: 3646, SEQ ID NO: 3647, SEQ ID NO:3648, or SEQ ID NO: 3649. In some embodiments, the first Fc region, the second Fc region, or a combination thereof comprises the sequence of SEQ ID NO: 40, SEQ ID NO: 42, SEQ ID NO: 3645, SEQ ID NO: 3646, SEQ ID NO: 3647, SEQ ID NO:3648, or SEQ ID NO: 3649.

In some embodiments, the sequence of the first Fc region, the second Fc region, or a combination thereof is a sequence having at least 75%, 80%, 85%, 90%, 95%, 99%, 99.5%, 99.9%, or 100% sequence identity to the sequence of SEQ ID NO: 40, SEQ ID NO: 42, SEQ ID NO: 3645, SEQ ID NO: 3646, SEQ ID NO: 3647, SEQ ID NO:3648, or SEQ ID NO: 3649. In some embodiments, the sequence of the first Fc region, the second Fc region, or a combination thereof is the sequence of SEQ ID NO: 40, SEQ ID NO: 42, SEQ ID NO: 3645, SEQ ID NO: 3646, SEQ ID NO: 3647, SEQ ID NO:3648, or SEQ ID NO: 3649.

In some embodiments, the first TCRαV-binding moiety and the second TCRαV-binding moiety are same. In some embodiments, the first TCRαV-binding moiety and the second TCRαV-binding moiety are different.

In some embodiments, the first TCRαV-binding moiety, the second TCRαV-binding moiety, or a combination thereof comprises: (i) a VH comprising a framework region (FR) comprising a framework 1 (FR1), a framework region 2 (FR2), a framework region 3 (FR3), and a framework region 4 (FR4) that have at least 75%, 80%, 85%, 90%, 95%, 99%, 99.5%, 99.9%, or 100% sequence identity with a non-murine germline FR1, a non-murine germline FR2, a non-murine germline FR3, and a non-murine germline FR4; (ii) a VL comprising a FR comprising a FR1, a FR2, a FR3, and a FR4 that have at least 75%, 80%, 85%, 90%, 95%, 99%, 99.5%, 99.9%, or 100% sequence identity with a non-murine germline FR1, a non-murine germline FR2, a non-murine germline FR3, and a non-murine germline FR4; or (iii) a combination thereof. In some embodiments, the first TCRαV-binding moiety, the second TCRαV-binding moiety, or a combination thereof comprises: (i) a VH comprising a FR comprising a FR1, a FR2, a FR3, and a FR4 having the sequences of a non-murine germline FR1, a non-murine germline FR2, a non-murine germline FR3, and a non-murine germline FR4; (ii) a VL comprising a FR comprising a FR1, a FR2, a FR3, and a FR4 having the sequences of a non-murine germline FR1, a non-murine germline FR2, a non-murine germline FR3, and a non-murine germline FR4; or (iii) a combination thereof.

In some embodiments, the first TCRαV-binding moiety, the second TCRαV-binding moiety, or a combination thereof comprises: (i) a VH comprising a FR1, a FR2, a FR3, and a FR4 that have at least 75%, 80%, 85%, 90%, 95%, 99%, 99.5%, 99.9%, or 100% sequence identity with a non-murine germline FR1, a non-murine germline FR2, a non-murine germline FR3, and a non-murine germline FR4, respectively; (ii) a VL comprising a FR comprising a FR1, a FR2, a FR3, and a FR4 that have at least 75%, 80%, 85%, 90%, 95%, 99%, 99.5%, 99.9%, or 100% sequence identity with a non-murine germline FR1, a non-murine germline FR2, a non-murine germline FR3, and a non-murine germline FR4, respectively; or
(iii) a combination thereof. In some embodiments, the first TCRαV-binding moiety, the second TCRαV-binding moiety, or a combination thereof comprises: (i) a VH comprising a FR comprising a FR1, a FR2, a FR3, and a FR4 having the sequences of a non-murine germline FR1, a non-murine germline FR2, a non-murine germline FR3, and a non-murine germline FR4, respectively; (ii) a VL comprising a FR comprising a FR1, a FR2, a FR3, and a FR4 having the sequences of a non-murine germline FR1, a non-murine germline FR2, a non-murine germline FR3, and a non-murine germline FR4, respectively; or (iii) a combination thereof.

In some embodiments, the first polypeptide, the second polypeptide, the third polypeptide, the fourth polypeptide, or a combination thereof comprises a heavy chain constant region having a sequence having at least 75%, 80%, 85%, 90%, 95%, 99%, 99.5%, 99.9%, or 100% sequence identity to any one of the sequences listed in Table 1 or a combination thereof. In some embodiments, the first polypeptide, the second polypeptide, the third polypeptide, the fourth polypeptide, or a combination thereof comprises a heavy chain constant region having any one of the sequences listed in Table 1 or a combination thereof. In some embodiments, the first polypeptide, the second polypeptide, the third polypeptide, the fourth polypeptide, or a combination thereof comprises a heavy chain constant region of which sequence is a sequence having at least 75%, 80%, 85%, 90%, 95%, 99%, 99.5%, 99.9%, or 100% sequence identity to any one of the sequences listed in Table 1 or a combination thereof. In some embodiments, the first polypeptide, the second polypeptide, the third polypeptide, the fourth polypeptide, or a combination thereof comprises a heavy chain constant region having any one of the heavy chain constant region sequences listed in Table 1 or a combination thereof. In some embodiments, the first polypeptide, the second polypeptide, the third polypeptide, the fourth polypeptide, or a combination thereof comprises a heavy chain constant region of an IgM or a fragment thereof. In some embodiments, the heavy chain constant region of the IgM comprises a sequence having at least 75%, 80%, 85%, 90%, 95%, 99%, 99.5%, 99.9%, or 100% sequence identity to the sequence of SEQ ID NO: 73. In some embodiments, the heavy chain constant region of the IgM comprises the sequence of SEQ ID NO: 73. In some embodiments, the sequence of the heavy chain constant region of the IgM is the sequence of SEQ ID NO: 73.

In some embodiments, the first polypeptide, the second polypeptide, the third polypeptide, the fourth polypeptide, or a combination thereof comprises a heavy chain constant region of an IgJ or a fragment thereof. In some embodiments, the heavy chain constant region of the IgJ comprises a sequence having at least 75%, 80%, 85%, 90%, 95%, 99%, 99.5%, 99.9%, or 100% sequence identity to the sequence of SEQ ID NO: 76. In some embodiments, the heavy chain constant region of the IgJ comprises the sequence of SEQ ID NO: 76. In some embodiments, the sequence of the heavy chain constant region of the IgJ is the sequence of SEQ ID NO: 76.

In some embodiments, the first polypeptide, the second polypeptide, the third polypeptide, the fourth polypeptide, or a combination thereof comprises a heavy chain constant region of an IgGA1 or a fragment thereof. In some embodiments, the heavy chain constant region of the IgGA1 comprises a sequence having at least 75%, 80%, 85%, 90%, 95%, 99%, 99.5%, 99.9%, or 100% sequence identity to the sequence of SEQ ID NO: 74. In some embodiments, the heavy chain constant region of the IgGA1 comprises the sequence of SEQ ID NO: 74. In some embodiments, the sequence of the heavy chain constant region of the IgGA1 is the sequence of SEQ ID NO: 74.

In some embodiments, the first polypeptide, the second polypeptide, the third polypeptide, the fourth polypeptide, or a combination thereof comprises a heavy chain constant region of an IgGA2 or a fragment thereof. In some embodiments, the heavy chain constant region of the IgGA2 comprises a sequence having at least 75, 80%, 85%, 90%, 95%, 99%, 99.5%, 99.9%, or 100% sequence identity to the sequence of SEQ ID NO: 75. In some embodiments, the heavy chain constant region of the IgGA2 comprises the sequence of SEQ ID NO: 75. In some embodiments, the sequence of the heavy chain constant region of the IgGA2 is the sequence of SEQ ID NO: 75.

In some embodiments, the first polypeptide, the second polypeptide, the third polypeptide, the fourth polypeptide, or a combination thereof comprises a heavy chain constant region of an IgG1 or a fragment thereof. In some embodiments, the heavy chain constant region of the IgG1 comprises a sequence having at least 75, 80%, 85%, 90%, 95%, 99%, 99.5%, 99.9%, or 100% sequence identity to the sequence of SEQ ID NO: 41. In some embodiments, the heavy chain constant region of the IgG1 comprises the sequence of SEQ ID NO: 41. In some embodiments, the sequence of the heavy chain constant region of the IgG1 is the sequence of SEQ ID NO: 41. In some embodiments, the heavy chain constant region of the IgG1 comprises a sequence having at least 75%, 80%, 85%, 90%, 95%, 99%, 99.5%, 99.9%, or 100% sequence identity to the sequence of SEQ ID NO: 3645. In some embodiments, the heavy chain constant region of the IgG1 comprises the sequence of SEQ ID NO: 3645. In some embodiments, the sequence of the heavy chain constant region of the IgG1 is the sequence of SEQ ID NO: 3645.

In some embodiments, the first polypeptide, the second polypeptide, the third polypeptide, the fourth polypeptide, or a combination thereof comprises a light chain constant region having a sequence having at least 75%, 80%, 85%, 90%, 95%, 99%, 99.5%, 99.9%, or 100% sequence identity to any one of the sequences listed in Table 1 or a combination thereof. In some embodiments, the first polypeptide, the second polypeptide, the third polypeptide, the fourth polypeptide, or a combination thereof comprises a light chain constant region having any one of the sequences listed in Table 3 or a combination thereof. In some embodiments, the first polypeptide, the second polypeptide, the third polypeptide, the fourth polypeptide, or a combination thereof comprises a light chain constant region having any one of the light chain constant region sequences listed in Table 3 or a combination thereof.

In some embodiments, the first polypeptide, the second polypeptide, the third polypeptide, the fourth polypeptide, or a combination thereof comprises a light chain constant region of a kappa chain or a fragment thereof. In some embodiments, the light chain constant region of a kappa chain comprises a light chain constant region sequence listed in Table 3.

In some embodiments, the light chain constant region of a kappa chain comprises a sequence having at least 75%, 80%, 85%, 90%, 95%, 99%, 99.5%, 99.9%, or 100% sequence identity to the sequence of SEQ ID NO: 39 or SEQ ID NO: 3644. In some embodiments, the light chain constant region of a kappa chain comprises the sequence of SEQ ID NO: 39 or SEQ ID NO: 3644. In some embodiments, the sequence of the light chain constant region of a kappa chain is a sequence having at least 75%, 80%, 85%, 90%, 95%, 99%, 99.5%, 99.9%, or 100% sequence identity to the sequence of SEQ ID NO: 39 or SEQ ID NO: 3644. In some embodiments, the sequence of the light chain constant region of a kappa chain is the sequence of SEQ ID NO: 39 or SEQ ID NO: 3644.

In some embodiments, the first TCRαV-binding moiety, the second TCRαV-binding moiety, or a combination thereof binds to an outward facing region on a TCRαV protein. In some embodiments, the outward facing region on the TCRαV protein comprises a structurally conserved region of TCRαV having a similar structure across one or more TCRαV subfamilies.

### Cytokine Molecules

In some embodiments, the multifunctional molecule includes a cytokine molecule. As used herein, a “cytokine molecule” or a “cytokine polypeptide” as interchangeably used herein, refers to full length, a fragment or a variant of a cytokine; a cytokine further comprising a receptor domain, e.g., a cytokine receptor dimerizing domain; or an agonist of a cytokine receptor, e.g., an antibody molecule (e.g., an agonistic antibody) to a cytokine receptor, that elicits at least one activity of a naturally-occurring cytokine. In some embodiments the cytokine molecule is chosen from interleukin-2 (IL-2), interleukin-7 (1L-7), interleukin-12 (IL-12), interleukin-10 (IL-10), interleukin-15 (IL-15), interleukin-18 (IL-18), interleukin-21 (IL-21), or interferon gamma, or a fragment or variant thereof, or a combination of any of the aforesaid cytokines. The cytokine molecule can be a monomer or a dimer. In embodiments, the cytokine molecule can further include a cytokine receptor dimerizing domain. In other embodiments, the cytokine molecule is an agonist of a cytokine receptor, e.g., an antibody molecule (e.g., an agonistic antibody) to a cytokine receptor chosen from an IL-15Ra or IL-21R.

Cytokines are generally polypeptides that influence cellular activity, for example, through signal transduction pathways. Accordingly, a cytokine of the multispecific or multifunctional polypeptide is useful and can be associated with receptor-mediated signaling that transmits a signal from outside the cell membrane to modulate a response within the cell. Cytokines are proteinaceous signaling compounds that are mediators of the immune response. They control many different cellular functions including proliferation, differentiation and cell survival/apoptosis; cytokines are also involved in several pathophysiological processes including viral infections and autoimmune diseases. Cytokines are synthesized under various stimuli by a variety of cells of both the innate (monocytes, macrophages, dendritic cells) and adaptive (T- and B-cells) immune systems. Cytokines can be classified into two groups: pro- and anti-inflammatory. Pro-inflammatory cytokines, including IFNγ, IL-1, IL-6 and TNF-alpha, are predominantly derived from the innate immune cells and Th1 cells. Anti-inflammatory cytokines, including IL-10, IL-4, IL-13 and IL-5, are synthesized from Th2 immune cells.

Provided herein are, inter alia, multispecific (e.g., bi-, tri-, quad-specific) or multifunctional molecules, that include, e.g., are engineered to contain, one or more cytokine molecules, e.g., immunomodulatory (e.g., proinflammatory) cytokines and variants, e.g., functional variants, thereof. Accordingly, in some embodiments, the cytokine molecule is an interleukin or a variant, e.g., a functional variant thereof. In some embodiments the interleukin is a proinflammatory interleukin. In some embodiments the interleukin is chosen from interleukin-2 (IL-2), interleukin-12 (IL-12), interleukin-15 (IL-15), interleukin-18 (IL-18), interleukin-21 (IL-21), interleukin-7 (IL-7), or interferon gamma. In some embodiments, the cytokine molecule is a proinflammatory cytokine.

In certain embodiments, the cytokine is a single chain cytokine. In certain embodiments, the cytokine is a multichain cytokine (e.g., the cytokine comprises 2 or more (e.g., 2) polypeptide chains. An exemplary multichain cytokine is IL-12.

Examples of useful cytokines include, but are not limited to, GM-CSF, IL-1a, IL-1p, IL-2, IL-3, IL-4, IL-5, IL-6, IL-7, IL-8, IL-10, IL-12, IL-21, IFN-α, IFN-β, IFN-γ, MIP-1α, MIP-1β, TGF-β, TNF-α, and TNFβ. In some embodiments the cytokine of the multispecific or multifunctional polypeptide is a cytokine selected from the group of GM-CSF, IL-2, IL-7, IL-8, IL-10, IL-12, IL-15, IL-21, IFN-α, IFN-γ, MIP-1α, MIP-1β and TGF-β. In some embodiments the cytokine of the i the multispecific or multifunctional polypeptide is a cytokine selected from the group of IL-2, IL-7, IL-10, IL-12, IL-15, IFN-α, and IFN-γ. In certain embodiments the cytokine is mutated to remove N- and/or O-glycosylation sites. Elimination of glycosylation increases homogeneity of the product obtainable in recombinant production.

In some embodiments, the cytokine of the multispecific or multifunctional polypeptide is IL-2. In a specific embodiment, the IL-2 cytokine can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity. In another particular embodiment, the IL-2 cytokine is a mutant IL-2 cytokine having reduced binding affinity to the .alpha.-subunit of the IL-2 receptor. Together with the .beta.- and .gamma.-subunits (also known as CD122 and CD132, respectively), the .alpha.-subunit (also known as CD25) forms the heterotrimeric high-affinity IL-2 receptor, while the dimeric receptor consisting only of the β- and γ-subunits is termed the intermediate-affinity IL-2 receptor. As described in PCT patent application number PCT/EP2012/051991, which is incorporated herein by reference in its entirety, a mutant IL-2 polypeptide with reduced binding to the .alpha.-subunit of the IL-2 receptor has a reduced ability to induce IL-2 signaling in regulatory T cells, induces less activation-induced cell death (AICD) in T cells, and has a reduced toxicity profile in vivo, compared to a wild-type IL-2 polypeptide. The use of such an cytokine with reduced toxicity is particularly advantageous in a multispecific or multifunctional polypeptide according to the invention, having a long serum half-life due to the presence of an Fc domain. In some embodiments, the mutant IL-2 cytokine of the multispecific or multifunctional polypeptide according to the invention comprises at least one amino acid mutation that reduces or abolishes the affinity of the mutant IL-2 cytokine to the .alpha.-subunit of the IL-2 receptor (CD25) but preserves the affinity of the mutant IL-2 cytokine to the intermediate-affinity IL-2 receptor (consisting of the β and γ subunits of the IL-2 receptor), compared to the non-mutated IL-2 cytokine. In some embodiments the one or more amino acid mutations are amino acid substitutions. In a specific embodiment, the mutant IL-2 cytokine comprises one, two or three amino acid substitutions at one, two or three position(s) selected from the positions corresponding to residue 42, 45, and 72 of human IL-2. In a more specific embodiment, the mutant IL-2 cytokine comprises three amino acid substitutions at the positions corresponding to residue 42, 45 and 72 of human IL-2. In an even more specific embodiment, the mutant IL-2 cytokine is human IL-2 comprising the amino acid substitutions F42A, Y45A and L72G. In some embodiments the mutant IL-2 cytokine additionally comprises an amino acid mutation at a position corresponding to position 3 of human IL-2, which eliminates the O-glycosylation site of IL-2. Particularly, said additional amino acid mutation is an amino acid substitution replacing a threonine residue by an alanine residue. A particular mutant IL-2 cytokine useful in the invention comprises four amino acid substitutions at positions corresponding to residues 3, 42, 45 and 72 of human IL-2. Specific amino acid substitutions are T3A, F42A, Y45A and L72G. As demonstrated in PCT patent application number PCT/EP2012/051991 and in the appended Examples, said quadruple mutant IL-2 polypeptide (IL-2 qm) exhibits no detectable binding to CD25, reduced ability to induce apoptosis in T cells, reduced ability to induce IL-2 signaling in T.sub.reg cells, and a reduced toxicity profile in vivo. However, it retains ability to activate IL-2 signaling in effector cells, to induce proliferation of effector cells, and to generate IFN-γ as a secondary cytokine by NK cells.

The IL-2 or mutant IL-2 cytokine according to any of the above embodiments may comprise additional mutations that provide further advantages such as increased expression or stability. For example, the cysteine at position 125 may be replaced with a neutral amino acid such as alanine, to avoid the formation of disulfide-bridged IL-2 dimers. Thus, in certain embodiments the IL-2 or mutant IL-2 cytokine of the multispecific or multifunctional polypeptide according to the invention comprises an additional amino acid mutation at a position corresponding to residue 125 of human IL-2. In some embodiments said additional amino acid mutation is the amino acid substitution C125A.

In a specific embodiment the IL-2 cytokine of the multispecific or multifunctional polypeptide comprises the polypeptide sequence of SEQ ID NO: 2270 [APTSSSTKKTQLQLEHLLLDLQMILNGINNYKNPKLTRMLTFKFYMPKKATELKHLQCLEEELK PLEEVLNLAQSKNFHLRPRDLISNINVIVLELKGSETITFMCEYADETATIVEFLNRWITFAQSIISTL T].

In another specific embodiment the IL-2 cytokine of the multispecific or multifunctional polypeptide comprises the polypeptide sequence of SEQ ID NO: 2280 [APASSSTKKTQLQLEHLLLDLQMILNGINNYKNPKLTRMLTAKFAMPKKATELKHLQCLEEELK PLEEVLNGAQSKNFHLRPRDLISNINVIVLELKGSETTFMCEYADETATIVEFLNRWITFAQSIISTL T].

In another embodiment the cytokine of the multispecific or multifunctional polypeptide is IL-12. In a specific embodiment said IL-12 cytokine is a single chain IL-12 cytokine. In an even more specific embodiment the single chain IL-12 cytokine comprises the polypeptide sequence of SEQ ID NO: 2290 [IWELKKDVYVVELDWYPDAPGEMVVLTCDTPEEDGITWTLDQSSEVLGSGKTLTIQVKEFGDA GQYTCHKGGEVLSHSLLLLHKKEDGIWSTDILKDQKEPKNKTFLRCEAKNYSGRFTCWWLTTIS TDLTFSVKSSRGSSDPQGVTCGAATLSAERVRGDNKEYEYSVECQEDSACPAAEESLPIEVMVD AVHKLKYENYTSSFFIRDIIKPDPPKNLQLKPLKNSRQVEVSWEYPDTWSTPHSYFSLTFCVQVQ GKSKREKKDRVFTDKTSATVICRKNASISVRAQDRYYSSSWSEWASVPCSGGGGSGGGGSGGG GSRNLPVATPDPGMFPCLHHSQNLLRAVSNMLQKARQTLEFYPCTSEEIDHEDITKDKTSTVEAC LPLELTKNESCLNSRETSFITNGSCLASRKTSFMMALCLSSIYEDLKMYQVEFKTMNAKLLMDPK RQIFLDQNMLAVIDELMQALNFNSETVPQKSSLEEPDFYKTKIKLCILLHAFRIRAVTIDRVMSYL NAS]. In some embodiments, the IL-12 cytokine can elicit one or more of the cellular responses selected from the group consisting of: proliferation in a NK cell, differentiation in a NK cell, proliferation in a T cell, and differentiation in a T cell.

In another embodiment the cytokine of the multispecific or multifunctional polypeptide is IL-10. In a specific embodiment said IL-10 cytokine is a single chain IL-10 cytokine. In an even more specific embodiment, the single chain IL-10 cytokine comprises the polypeptide sequence of SEQ ID NO: 2300 [SPGQGTQSENSCTHFPGNLPNMLRDLRDAFSRVKTFFQMKDQLDNLLLKESLLEDFKGYLGCQ ALSEMIQFYLEEVMPQAENQDPDIKAHVNSLGENLKTLRLRLRRCHRFLPCENKSKAVEQVKNA FNKLQEKGIYKAMSEFDIFINYIEAYMTMKIRNGGGGSGGGGSGGGGSGGGGSSPGQGTQSENS CTHFPGNLPNMLRDLRDAFSRVKTFFQMKDQLDNLLLKESLLEDFKGYLGCQALSEMIQFYLEE VMPQAENQDPDIKAHVNSLGENLKTLRLRLRRCHRFLPCENKSKAVEQVKNAFNKLQEKGIYK AMSEFDIFINYIEAYMTMKIRN].

In another specific embodiment the IL-10 cytokine is a monomeric IL-10 cytokine. In a more specific embodiment the monomeric IL-10 cytokine comprises the polypeptide sequence of SEQ ID NO: 2310 [SPGQGTQSENSCTHFPGNLPNMLRDLRDAFSRVKTFFQMKDQLDNLLLKESLLEDFKGYLGCQ ALSEMIQFYLEEVMPQAENQDPDIKAHVNSLGENLKTLRLRLRRCHRFLPCENGGGSGGKSKAV EQVKNAFNKLQEKGIYKAMSEFDIFINYIEAYMTMKIRN]. In some embodiments, the IL-10 cytokine can elicit one or more of the cellular responses selected from the group consisting of: inhibition of cytokine secretion, inhibition of antigen presentation by antigen presenting cells, reduction of oxygen radical release, and inhibition of T cell proliferation. A multispecific or multifunctional polypeptide according to the invention wherein the cytokine is IL-10 is particularly useful for downregulation of inflammation, e.g. in the treatment of an inflammatory disorder.

In another embodiment, the cytokine of the multispecific or multifunctional polypeptide is IL-15. In a specific embodiment said IL-15 cytokine is a mutant IL-15 cytokine having reduced binding affinity to the a-subunit of the IL-15 receptor. Without wishing to be bound by theory, a mutant IL-15 polypeptide with reduced binding to the .alpha.-subunit of the IL-15 receptor has a reduced ability to bind to fibroblasts throughout the body, resulting in improved pharmacokinetics and toxicity profile, compared to a wild-type IL-15 polypeptide. The use of an cytokine with reduced toxicity, such as the described mutant IL-2 and mutant IL-15 effector moieties, is particularly advantageous in a multispecific or multifunctional polypeptide according to the invention, having a long serum half-life due to the presence of an Fc domain. In some embodiments the mutant IL-15 cytokine of the multispecific or multifunctional polypeptide according to the invention comprises at least one amino acid mutation that reduces or abolishes the affinity of the mutant IL-15 cytokine to the alpha.-subunit of the IL-15 receptor but preserves the affinity of the mutant IL-15 cytokine to the intermediate-affinity IL-15/IL-2 receptor (consisting of the .beta.- and .gamma.-subunits of the IL-15/IL-2 receptor), compared to the non-mutated IL-15 cytokine. In some embodiments the amino acid mutation is an amino acid substitution. In a specific embodiment, the mutant IL-15 cytokine comprises an amino acid substitution at the position corresponding to residue 53 of human IL-15. In a more specific embodiment, the mutant IL-15 cytokine is human IL-15 comprising the amino acid substitution E53A. In some embodiments the mutant IL-15 cytokine additionally comprises an amino acid mutation at a position corresponding to position 79 of human IL-15, which eliminates the N-glycosylation site of IL-15. Particularly, said additional amino acid mutation is an amino acid substitution replacing an asparagine residue by an alanine residue. In an even more specific embodiment, the IL-15 cytokine comprises the polypeptide sequence of SEQ ID NO: 2320 [NWVNVISDLKKIEDLIQSMHIDATLYTESDVHPSCKVTAMKCFLLELQVISLASGDASIHDTVEN LIILANNSLSSNGAVTESGCKECEELEEKNIKEFLQSFVHIVQMFINTS]. In some embodiments, the IL-15 cytokine can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity.

Mutant cytokine molecules useful as effector moieties in the multispecific or multifunctional polypeptide can be prepared by deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing. Substitution or insertion may involve natural as well as non-natural amino acid residues. Amino acid modification includes well known methods of chemical modification such as the addition or removal of glycosylation sites or carbohydrate attachments, and the like.

In some embodiments, the cytokine, particularly a single-chain cytokine, of the multispecific or multifunctional polypeptide is GM-CSF. In a specific embodiment, the GM-CSF cytokine can elicit proliferation and/or differentiation in a granulocyte, a monocyte or a dendritic cell. In some embodiments, the cytokine, particularly a single-chain cytokine, of the multispecific or multifunctional polypeptide is IFN-α. In a specific embodiment, the IFN-α cytokine can elicit one or more of the cellular responses selected from the group consisting of: inhibiting viral replication in a virus-infected cell, and upregulating the expression of major histocompatibility complex I (MHC I). In another specific embodiment, the IFN-α cytokine can inhibit proliferation in a tumor cell. In some embodiments the cytokine, particularly a single-chain cytokine, of the multispecific or multifunctional polypeptide is IFNγ. In a specific embodiment, the IFN-γ cytokine can elicit one or more of the cellular responses selected from the group of: increased macrophage activity, increased expression of MHC molecules, and increased NK cell activity. In some embodiments the cytokine, particularly a single-chain cytokine, of the multispecific or multifunctional polypeptide is IL-7. In a specific embodiment, the IL-7 cytokine can elicit proliferation of T and/or B lymphocytes. In some embodiments, the cytokine, particularly a single-chain cytokine, of the multispecific or multifunctional polypeptide is IL-8. In a specific embodiment, the IL-8 cytokine can elicit chemotaxis in neutrophils. In some embodiments, the cytokine, particularly a single-chain cytokine, of the multispecific or multifunctional polypeptide, is MIP-1α. In a specific embodiment, the MIP-1α cytokine can elicit chemotaxis in monocytes and T lymphocyte cells. In some embodiments, the cytokine, particularly a single-chain cytokine, of the multispecific or multifunctional polypeptide is MIP-1β. In a specific embodiment, the MIP-1β cytokine can elicit chemotaxis in monocytes and T lymphocyte cells. In some embodiments, the cytokine, particularly a single-chain cytokine, of the multispecific or multifunctional polypeptide is TGF-β. In a specific embodiment, the TGF-βcytokine can elicit one or more of the cellular responses selected from the group consisting of: chemotaxis in monocytes, chemotaxis in macrophages, upregulation of IL-1 expression in activated macrophages, and upregulation of IgA expression in activated B cells.

In some embodiments, the multispecific or multifunctional polypeptide of the invention binds to a cytokine receptor with a dissociation constant (K_{D}) that is at least about 1, 1.5, 2, 2.5, 3, 3.5, 4, 4.5, 5, 5.5, 6, 6.5, 7, 7.5, 8, 8.5, 9, 9.5 or 10 times greater than that for a control cytokine. In another embodiment, the multispecific or multifunctional polypeptide binds to a cytokine receptor with a K_{D} that is at least 2, 3, 4, 5, 6, 7, 8, 9, or 10 times greater than that for a corresponding multispecific or multifunctional polypeptide comprising two or more effector moieties. In another embodiment, the multispecific or multifunctional polypeptide binds to a cytokine receptor with a dissociation constant K_{D} that is about 10 times greater than that for a corresponding the multispecific or multifunctional polypeptide comprising two or more cytokines.

In some embodiments, the multispecific molecules as described herein include a cytokine molecule. In embodiments, the cytokine molecule includes a full length, a fragment or a variant of a cytokine; a cytokine receptor domain, e.g., a cytokine receptor dimerizing domain; or an agonist of a cytokine receptor, e.g., an antibody molecule (e.g., an agonistic antibody) to a cytokine receptor.

In some embodiments the cytokine molecule is chosen from IL-2, IL-12, IL-15, IL-18, IL-7, IL-21, or interferon gamma, or a fragment or variant thereof, or a combination of any of the aforesaid cytokines. The cytokine molecule can be a monomer or a dimer. In embodiments, the cytokine molecule can further include a cytokine receptor dimerizing domain.

In other embodiments, the cytokine molecule is an agonist of a cytokine receptor, e.g., an antibody molecule (e.g., an agonistic antibody) to a cytokine receptor chosen from an IL-15Ra or IL-21R.

In some embodiments, the cytokine molecule is IL-15, e.g., human IL-15 (e.g., comprising the amino acid sequence: NWVNVISDLKKIEDLIQSMHIDATLYTESDVHPSCKVTAMKCFLLELQVISLESGDASIHDTVENL IILANNSLSSNGNVTESGCKECEELEEKNIKEFLQSFVHIVQMFINTS (SEQ ID NO: 2170), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 2170.

In some embodiments, the cytokine molecule comprises a receptor dimerizing domain, e.g., an IL15Ralpha dimerizing domain. In some embodiments, the IL15Ralpha dimerizing domain comprises the amino acid sequence: MAPRRARGCRTLGLPALLLLLLLRPPATRGITCPPPMSVEHADIWVKSYSLYSRERYICNSGFKR KAGTSSLTECVL (SEQ ID NO: 2180), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 2180. In some embodiments, the cytokine molecule (e.g., IL-15) and the receptor dimerizing domain (e.g., an IL15Ralpha dimerizing domain) of the multispecific molecule are covalently linked, e.g., via a linker (e.g., a Gly-Ser linker, e.g., a linker comprising the amino acid sequence SGGSGGGGSGGGSGGGGSLQ (SEQ ID NO: 2190). In other embodiments, the cytokine molecule (e.g., IL-15) and the receptor dimerizing domain (e.g., an IL15Ralpha dimerizing domain) of the multispecific molecule are not covalently linked, e.g., are non-covalently associated.

In other embodiments, the cytokine molecule is IL-2, e.g., human IL-2 (e.g., comprising the amino acid sequence: APTSSSTKKTQLQLEHLLLDLQMILNGINNYKNPKLTRMLTFKFYMPKKATELKHLQCLEEELKP LEEVLNLAQSKNFHLRPRDLISNINVIVLELKGSETTFMCEYADETATIVEFLNRWITFCQSIISTLT (SEQ ID NO: 2191), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO:2191).

In other embodiments, the cytokine molecule is IL-18, e.g., human IL-18 (e.g., comprising the amino acid sequence: YFGKLESKLSVIRNLNDQVLFIDQGNRPLFEDMTDSDCRDNAPRTIFIlSMYKDSQPRGMAVTISV KCEKISTLSCENKIISFKEMNPPDNIKDTKSDIIFFQRSVPGHDNKMQFESSSYEGYFLACEKERDL FKLILKKEDELGDRSIMFTVQNED (SEQ ID NO: 2192), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 2192).

In other embodiments, the cytokine molecule is IL-21, e.g., human IL-21 (e.g., comprising the amino acid sequence: QGQDRHMIRMRQLIDIVDQLKNYVNDLVPEFLPAPEDVETNCEWSAFSCFQKAQLKSANTGNNE RIINVSIKKLKRKPPSTNAGRRQKHRLTCPSCDSYEKKPPKEFLERFKSLLQKMIHQHLSSRTHGS EDS (SEQ ID NO: 2193), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 2193).

In yet other embodiments, the cytokine molecule is interferon gamma, e.g., human interferon gamma (e.g., comprising the amino acid sequence: QDPYVKEAENLKKYFNAGHSDVADNGTLFLGILKNWKEESDRKIMQSQIVSFYFKLFKNFKDDQ SIQKSVETIKEDMNVKFFNSNKKKRDDFEKLTNYSVTDLNVQRKAIHELIQVMAELSPAAKTGK RKRSQMLFRG (SEQ ID NO: 2194), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 2194).

### Immune Cell Engagers

In some embodiments, the multifunctional molecule further includes an immune cell engager. “An immune cell engager” refers to one or more binding specificities that bind and/or activate an immune cell, e.g., a cell involved in an immune response. In embodiments, the immune cell is chosen from a T cell, an NK cell, a B cell, a dendritic cell, and/or the macrophage cell. The immune cell engager can be an antibody molecule, a receptor molecule (e.g., a full length receptor, receptor fragment, or fusion thereof (e.g., a receptor-Fc fusion)), or a ligand molecule (e.g., a full length ligand, ligand fragment, or fusion thereof (e.g., a ligand-Fc fusion)) that binds to the immune cell antigen (e.g., the T cell, the NK cell antigen, the B cell antigen, the dendritic cell antigen, and/or the macrophage cell antigen). In embodiments, the immune cell engager specifically binds to the target immune cell, e.g., binds preferentially to the target immune cell. For example, when the immune cell engager is an antibody molecule, it binds to an immune cell antigen (e.g., a T cell antigen, an NK cell antigen, a B cell antigen, a dendritic cell antigen, and/or a macrophage cell antigen) with a dissociation constant of less than about 10 nM.

The immune cell engagers, e.g., first and/or second immune cell engager, of the multispecific or multifunctional molecules as described herein can mediate binding to, and/or activation of, an immune cell, e.g., an immune effector cell. In some embodiments, the immune cell is chosen from a T cell, an NK cell, a B cell, a dendritic cell, or a macrophage cell engager, or a combination thereof. In some embodiments, the immune cell engager is chosen from one, two, three, or all of a T cell engager, NK cell engager, a B cell engager, a dendritic cell engager, or a macrophage cell engager, or a combination thereof. The immune cell engager can be an agonist of the immune system. In some embodiments, the immune cell engager can be an antibody molecule, a ligand molecule (e.g., a ligand that further comprises an immunoglobulin constant region, e.g., an Fc region), a small molecule, a nucleotide molecule.

### Natural Killer Cell Engagers:

Natural Killer (NK) cells recognize and destroy tumors and virus-infected cells in an antibody-independent manner. The regulation of NK cells is mediated by activating and inhibiting receptors on the NK cell surface. One family of activating receptors is the natural cytotoxicity receptors (NCRs) which include NKp30, NKp44 and NKp46. The NCRs initiate tumor targeting by recognition of heparan sulfate on cancer cells. NKG2D is a receptor that provides both stimulatory and costimulatory innate immune responses on activated killer (NK) cells, leading to cytotoxic activity. DNAM1 is a receptor involved in intercellular adhesion, lymphocyte signaling, cytotoxicity and lymphokine secretion mediated by cytotoxic T-lymphocyte (CTL) and NK cell. DAP10 (also known as HCST) is a transmembrane adapter protein which associates with KLRK1 to form an activation receptor KLRK1-HCST in lymphoid and myeloid cells; this receptor plays a major role in triggering cytotoxicity against target cells expressing cell surface ligands such as MHC class I chain-related MICA and MICB, and U (optionally L1)6-binding proteins (ULBPs); it KLRK1-HCST receptor plays a role in immune surveillance against tumors and is required for cytolysis of tumors cells; indeed, melanoma cells that do not express KLRK1 ligands escape from immune surveillance mediated by NK cells. CD16 is a receptor for the Fc region of IgG, which binds complexed or aggregated IgG and also monomeric IgG and thereby mediates antibody-dependent cellular cytotoxicity (ADCC) and other antibody-dependent responses, such as phagocytosis.

In some embodiments, the NK cell engager is a viral hemagglutinin (HA), HA is a glycoprotein found on the surface of influenza viruses. It is responsible for binding the virus to cells with sialic acid on the membranes, such as cells in the upper respiratory tract or erythrocytes. HA has at least 18 different antigens. These subtypes are named H1 through H18. NCRs can recognize viral proteins. NKp46 has been shown to be able to interact with the HA of influenza and the HA-NA of Paramyxovirus, including Sendai virus and Newcastle disease virus. Besides NKp46, NKp44 can also functionally interact with HA of different influenza subtypes.

Provided herein are, inter alia, multispecific (e.g., bi-, tri-, quad-specific) or multifunctional molecules that are engineered to contain one or more NK cell engagers that mediate binding to and/or activation of an NK cell. Accordingly, in some embodiments, the NK cell engager is selected from an antigen binding domain or ligand that binds to (e.g., activates); NKp30, NKp40, NKp44, NKp46, NKG2D, DNAM1, DAP10, CD16 (e.g., CD16a, CD16b, or both), CRTAM, CD27, PSGL1, CD96, CD100 (SEMA4D), NKp80, CD244 (also known as SLAMF4 or 2B4), SLAMF6, SLAMF7, KIR2DS2, KIR2DS4, KIR3DS1, KIR2DS3, KIR2DS5, KIR2DS1, CD94, NKG2C, NKG2E, or CD160.

In some embodiments, the NK cell engager is a ligand of NKp30 is a B7-6, e.g., comprises the amino acid sequence of: DLKVEMMAGGTQITPLNDNVTIFCNIFYSQPLNITSMGITWFWKSLTFDKEVKVFEFFGDHQEAF RPGAIVSPWRLKSGDASLRLPGIQLEEAGEYRCEVVVTPLKAQGTVQLEVVASPASRLLLDQVG MKENEDKYMCESSGFYPEAINITWEKQTQKFPHPIEISEDVITGPTIKNMDGTFNVTSCLKLNSSQ EDPGTVYQCVVRHASLHTPLRSNFTLTAARHSLSETEKTDNFS (SEQ ID NO: 3291), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3291.

In other embodiments, the NK cell engager is a ligand of NKp44 or NKp46, which is a viral HA. Viral hemagglutinins (HA) are glyco proteins which are on the surface of viruses. HA proteins allow viruses to bind to the membrane of cells via sialic acid sugar moieties which contributes to the fusion of viral membranes with the cell membranes (see e.g., Eur J Immunol. 2001 September; 31(9):2680-9 “Recognition of viral hemagglutinins by NKp44 but not by NKp30”; and Nature. 2001 Feb. 22; 409(6823):1055-60 “Recognition of haemagglutinins on virus-infected cells by NKp46 activates lysis by human NK cells” the contents of each of which are incorporated by reference herein).

In other embodiments, the NK cell engager is a ligand of NKG2D chosen from MICA, MICB, or ULBP1, e.g., wherein: (i) MICA comprises the amino acid sequence: EPHSLRYNLTVLSWDGSVQSGFLTEVHLDGQPFLRCDRQKCRAKPQGQWAEDVLGNKTWDRE TRDLTGNGKDLRMTLAHIKDQKEGLHSLQEIRVCEIHEDNSTRSSQHFYYDGELFLSQNLETKEW TMPQSSRAQTLAMNVRNFLKEDAMKTKTHYHAMHADCLQELRRYLKSGVVLRRTVPPMVNVT RSEASEGNITVTCRASGFYPWNITLSWRQDGVSLSHDTQQWGDVLPDGNGTYQTWVATRICQG EEQRFTCYMEHSGNHSTHPVPSGKVLVLQSHW (SEQ ID NO: 3292), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3292. (ii) MICB comprises the amino acid sequence: AEPHSLRYNLMVLSQDESVQSGFLAEGHLDGQPFLRYDRQKRRAKPQGQWAEDVLGAKTWDT ETEDLTENGQDLRRTLTHIKDQKGGLHSLQEIRVCEIHEDSSTRGSRHFYYDGELFLSQNLETQES TVPQSSRAQTLAMNVTNFWKEDAMKTKTHYRAMQADCLQKLQRYLKSGVAIRRTVPPMVNVT CSEVSEGNITVTCRASSFYPRNITLTWRQDGVSLSHNTQQWGDVLPDGNGTYQTWVATRIRQGE EQRFTCYMEHSGNHGTHPVPSGKVLVLQSQRTD (SEQ ID NO: 3293), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3293; or (iii) ULBP1 comprises the amino acid sequence: GWVDTHCLCYDFIITPKSRPEPQWCEVQGLVDERPFLHYDCVNHKAKAFASLGKKVNVTKTWE EQTETLRDVVDFLKGQLLDIQVENLIPIEPLTLQARMSCEHEAHGHGRGSWQFLFNGQKFLLFDS NNRKWTALHPGAKKMTEKWEKNRDVTMFFQKISLGDCKMWLEEFLMYWEQMLDPTKPPSLA PG (SEQ ID NO: 3294), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3294.

In other embodiments, the NK cell engager is a ligand of DNAM1 chosen from NECTIN2 or NECL5, e.g., wherein: (i) NECTIN2 comprises the amino acid sequence: QDVRVQVLPEVRGQLGGTVELPCHLLPPVPGLYISLVTWQRPDAPANHQNVAAFHPKMGPSFPS PKPGSERLSFVSAKQSTGQDTEAELQDATLALHGLTVEDEGNYTCEFATFPKGSVRGMTWLRVI AKPKNQAEAQKVTFSQDPTTVALCISKEGRPPARISWLSSLDWEAKETQVSGTLAGTVTVTSRFT LVPSGRADGVTVTCKVEHESFEEPALIPVTLSVRYPPEVSISGYDDNWYLGRTDATLSCDVRSNP EPTGYDWSTTSGTFPTSAVAQGSQLVIHAVDSLFNTTFVCTVTNAVGMGRAEQVIFVRETPNTA GAGATGG (SEQ ID NO: 3295), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3295; or (ii) NECL5 comprises the amino acid sequence: WPPPGTGDVVVQAPTQVPGFLGDSVTLPCYLQVPNMEVTHVSQLTWARHGESGSMAVFHQTQ GPSYSESKRLEFVAARLGAELRNASLRMFGLRVEDEGNYTCLFVTFPQGSRSVDIWLRVLAKPQ NTAEVQKVQLTGEPVPMARCVSTGGRPPAQITWHSDLGGMPNTSQVPGFLSGTVTVTSLWILVP SSQVDGKNVTCKVEHESFEKPQLLTVNLTVYYPPEVSISGYDNNWYLGQNEATLTCDARSNPEP TGYNWSTTMGPLPPFAVAQGAQLLIRPVDKPINTTLICNVTNALGARQAELTVQVKEGPPSEHSG ISRN (SEQ ID NO: 3296), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3296.

In yet other embodiments, the NK cell engager is a ligand of DAP10, which is an adapter for NKG2D (see e.g., Proc Nat Acad Sci USA. 2005 May 24; 102(21): 7641-7646, and Blood, 15 Sep. 2011 Volume 118, Number 11, the full contents of each of which is incorporated by reference herein).

In other embodiments, the NK cell engager is a ligand of CD16, which is a CD16a/b ligand, e.g., a CD16a/b ligand further comprising an antibody Fc region (see e.g., Front Immunol. 2013; 4: 76 discusses how antibodies use the Fc to trigger NK cells through CD16, the full contents of which are incorporated herein).

In other embodiments, the NK cell engager is a ligand of CRTAM, which is NECL2, e.g., wherein NECL2 comprises the amino acid sequence: QNLFTKDVTVIEGEVATISCQVNKSDDSVIQLLNPNRQTIYFRDFRPLKDSRFQLLNFSSSELKVSL TNVSISDEGRYFCQLYTDPPQESYTTITVLVPPRNLMIDIQKDTAVEGEEIEVNCTAMASKPATTIR WFKGNTELKGKSEVEEWSDMYTVTSQLMLKVHKEDDGVPVICQVEHPAVTGNLQTQRYLEVQ YKPQVHIQMTYPLQGLTREGDALELTCEAIGKPQPVMVTWVRVDDEMPQHAVLSGPNLFINNL NKTDNGTYRCEASNIVGKAHSDYMLYVYDPPTTIPPPI IIIT ILTIITDSRAGEEGSIR AVDH (SEQ ID NO: 3297), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3297.

In other embodiments, the NK cell engager is a ligand of CD27, which is CD70, e.g., wherein CD70 comprises the amino acid sequence: QRFAQAQQQLPLESLGWDVAELQLNHTGPQQDPRLYWQGGPALGRSFLHGPELDKGQLRIHRD GIYMVHIQVTLAICSSTTASRHHPTTLAVGICSPASRSISLLRLSFHQGCTIASQRLTPLARGDTLCT NLTGTLLPSRNTDETFFGVQWVRP (SEQ ID NO: 3298), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3298.

In other embodiments, the NK cell engager is a ligand of PSGL1, which is L-selectin (CD62L), e.g., wherein L-selectin comprises the amino acid sequence: WTYHYSEKPMNWQRARRFCRDNYTDLVAIQNKAEIEYLEKTLPFSRSYYWIGIRKIGGIWTWVG TNKSLTEEAENWGDGEPNNKKNKEDCVEIYIKRNKDAGKWNDDACHKLKAALCYTASCQPWS CSGHGECVEIINNYTCNCDVGYYGPQCQFVIQCEPLEAPELGTMDCTHPLGNFSFSSQCAFSCSEG TNLTGIEETTCGPFGNWSSPEPTCQVIQCEPLSA PDLGIMNCSHPLA SFSFTSACTFICSEGTELIGK KKTICESSGIWSNPSPICQKLDKSFSMIKEGDYN (SEQ ID NO: 3299), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3299.

In other embodiments, the NK cell engager is a ligand of CD96, which is NECL5, e.g., wherein NECL5 comprises the amino acid sequence: WPPPGTGDVVVQAPTQVPGFLGDSVTLPCYLQVPNMEVTHVSQLTWARHGESGSMAVFHQTQ GPSYSESKRLEFVAARLGAELRNASLRMFGLRVEDEGNYTCLFVTFPQGSRSVDIWLRVLAKPQ NTAEVQKVQLTGEPVPMARCVSTGGRPPAQITWHSDLGGMPNTSQVPGFLSGTVTVTSLWILVP SSQVDGKNVTCKVEHESFEKPQLLTVNLTVYYPPEVSISGYDNNWYLGQNEATLTCDARSNPEP TGYNWSTTMGPLPPFAVAQGAQLLIRPVDKPINTTLICNVTNALGARQAELTVQVKEGPPSEHSG ISRN (SEQ ID NO: 3296), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3296.

In other embodiments, the NK cell engager is a ligand of CD100 (SEMA4D), which is CD72, e.g., wherein CD72 comprises the amino acid sequence:

(SEQ ID NO: 3300)
RYLQVSQQLQQTNRVLEVINSSLRQQLRLKITQLGQSAEDLQGSRRELA
QSQEALQVEQRAHQAAEGQLQACQADRQKTKETLQSEEQQRRALEQKLS
NMENRLKPFFTCGSADTCCPSGWIMHQKSCFYISLTSKNWQESQKQCET
LSSKLATFSEIYPQSHSYYFLNSLLPNGGSGNSYWTGLSSNKDWKLTDD
TQRTRTYAQSSKCNKVHKTWSWWTLESESCRSSLPYICEMTAFRFPD,

a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3300.

In other embodiments, the NK cell engager is a ligand of NKp80, which is CLEC2B (AICL), e.g., wherein CLEC2B (AICL) comprises the amino acid sequence: KLTRDSQSLCPYDWIGFQNKCYYFSKEEGDWNSSKYNCSTQHADLTIIDNIEEMNFLRRYKCSSD HWIGLKMAKNRTGQWVDGATFTKSFGMRGSEGCAYLSDDGAATARCYTERKWICRKRIH (SEQ ID NO: 3301), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3301.

In other embodiments, the NK cell engager is a ligand of CD244, which is CD48, e.g., wherein CD48 comprises the amino acid sequence: QGHLVHMTVVSGSNVTLNISESLPENYKQLTWFYTFDQKIVEWDSRKSKYFESKFKGRVRLDPQ SGALYISKVQKEDNSTYIMRVLKKTGNEQEWKIKLQVLDPVPKPVIKIEKIEDMDDNCYLKLSCV IPGESVNYTWYGDKRPFPKELQNSVLETTLMPHNYSRCYTCQVSNSVSSKNGTVCLSPPCTLARS (SEQ ID NO: 3302), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions. e.g., conservative substitutions) to the amino acid sequence of SEQ ID NO: 3302.

### T Cell Engagers

Provided herein are, inter alia, multispecific (e.g., bi-, tri-, quad-specific) or multifunctional molecules that are engineered to further contain one or more T cell engager that mediate binding to and/or activation of a T cell. In some embodiments, the T cell engager is an antigen binding domain that binds to, e.g., activates TCRβ, e.g., a TCRβV region, as described herein. In some embodiments, the T cell engager is selected from an antigen binding domain or ligand that binds to (e.g., and in some embodiments activates) one or more of CD3, TCRα, TCRγ, TCRζ, ICOS, CD28, CD27, HVEM, LIGHT, CD40, 4-1BB, OX40, DR3, GITR, CD30, TIM1, SLAM, CD2, or CD226. In other embodiments, the T cell engager is selected from an antigen binding domain or ligand that binds to and does not activate one or more of CD3, TCRα, TCRγ, TCRζ, ICOS, CD28, CD27, HVEM, LIGHT, CD40, 4-1BB, OX40, DR3, GITR, CD30, TIM1, SLAM, CD2, or CD226.

### B Cell, Macrophage & Dendritic Cell Engagers

Broadly, B cells, also known as B lymphocytes, are a type of white blood cell of the lymphocyte subtype. They function in the humoral immunity component of the adaptive immune system by secreting antibodies. Additionally, B cells present antigen (they are also classified as professional antigen-presenting cells (APCs)) and secrete cytokines. Macrophages are a type of white blood cell that engulfs and digests cellular debris, foreign substances, microbes, cancer cells via phagocytosis. Besides phagocytosis, they play important roles in nonspecific defense (innate immunity) and also help initiate specific defense mechanisms (adaptive immunity) by recruiting other immune cells such as lymphocytes. For example, they are important as antigen presenters to T cells. Beyond increasing inflammation and stimulating the immune system, macrophages also play an important anti-inflammatory role and can decrease immune reactions through the release of cytokines. Dendritic cells (DCs) are antigen-presenting cells that function in processing antigen material and present it on the cell surface to the T cells of the immune system.

Provided herein are, inter alia, multispecific (e.g., bi-, tri-, quad-specific) or multifunctional molecules that further include, e.g., are engineered to contain, one or more B cell, macrophage, and/or dendritic cell engager that mediate binding to and/or activation of a B cell, macrophage, and/or dendritic cell.

Accordingly, in some embodiments, the immune cell engager comprises a B cell, macrophage, and/or dendritic cell engager chosen from one or more of CD40 ligand (CD40L) or a CD70 ligand; an antibody molecule that binds to CD40 or CD70; an antibody molecule to OX40; an OX40 ligand (OX40L); an agonist of a Toll-like receptor (e.g., as described herein, e.g., a TLR4, e.g., a constitutively active TLR4 (caTLR4), or a TLR9 agonists); a 41BB; a CD2; a CD47; or a STING agonist, or a combination thereof.

In some embodiments, the B cell engager is a CD40L, an OX40L, or a CD70 ligand, or an antibody molecule that binds to OX40, CD40 or CD70.

In some embodiments, the macrophage engager is a CD2 agonist. In some embodiments, the macrophage engager is an antigen binding domain that binds to: CD40L or antigen binding domain or ligand that binds CD40, a Toll like receptor (TLR) agonist (e.g., as described herein), e.g., a TLR9 or TLR4 (e.g., caTLR4 (constitutively active TLR4), CD47, or a STING agonist. In some embodiments, the STING agonist is a cyclic dinucleotide, e.g., cyclic di-GMP (cdGMP) or cyclic di-AMP (cdAMP). In some embodiments, the STING agonist is biotinylated.

In some embodiments, the dendritic cell engager is a CD2 agonist. In some embodiments, the dendritic cell engager is a ligand, a receptor agonist, or an antibody molecule that binds to one or more of: OX40L, 41BB, a TLR agonist (e.g., as described herein) (e.g., TLR9 agonist, TLR4 (e.g., caTLR4 (constitutively active TLR4)), CD47, or and a STING agonist. In some embodiments, the STING agonist is a cyclic dinucleotide, e.g., cyclic di-GMP (cdGMP) or cyclic di-AMP (cdAMP). In some embodiments, the STING agonist is biotinylated.

In other embodiments, the immune cell engager mediates binding to, or activation of, one or more of a B cell, a macrophage, and/or a dendritic cell. Exemplary B cell, macrophage, and/or dendritic cell engagers can be chosen from one or more of CD40 ligand (CD40L) or a CD70 ligand; an antibody molecule that binds to CD40 or CD70; an antibody molecule to OX40; an OX40 ligand (OX40L); a Toll-like receptor agonist (e.g., a TLR4, e.g., a constitutively active TLR4 (caTLR4) or a TLR9 agonist); a 41BB agonist; a CD2; a CD47; or a STING agonist, or a combination thereof.

In some embodiments, the B cell engager is chosen from one or more of a CD40L, an OX40L, or a CD70 ligand, or an antibody molecule that binds to OX40, CD40 or CD70.

In other embodiments, the macrophage cell engager is chosen from one or more of a CD2 agonist; a CD40L; an OX40L; an antibody molecule that binds to OX40, CD40 or CD70; a Toll-like receptor agonist or a fragment thereof (e.g., a TLR4, e.g., a constitutively active TLR4 (caTLR4)); a CD47 agonist; or a STING agonist.

In other embodiments, the dendritic cell engager is chosen from one or more of a CD2 agonist, an OX40 antibody, an OX40L, 41BB agonist, a Toll-like receptor agonist or a fragment thereof (e.g., a TLR4, e.g., a constitutively active TLR4 (caTLR4)), CD47 agonist, or a STING agonist.

In some embodiments, the OX40L comprises the amino acid sequence: QVSHRYPRIQSIKVQFTEYKKEKGFILTSQKEDEIMKVQNNSVIINCDGFYLISLKGYFSQEVNISL HYQKDEEPLFQLKKVRSVNSLMVASLTYKDKVYLNVTDNTSLDDFHVNGGELILIHQNPGEFC VL (SEQ ID NO: 3303), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3303.

In another embodiment, the CD40L comprises the amino acid sequence: MQKGDQNPQIAAHVISEASSKTTSVLQWAEKGYYTMSNNLVTLENGKQLTVKRQGLYYIYAQV TFCSNREASSQAPFIASLCLKSPGRFERILLRAANTHSSAKPCGQQSIHLGGVFELQPGASVFVNVT DPSQVSHGTGFTSFGLLKL (SEQ ID NO: 3304), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3304.

In yet other embodiments, the STING agonist comprises a cyclic dinucleotide, e.g., a cyclic di-GMP (cdGMP), a cyclic di-AMP (cdAMP), or a combination thereof, optionally with 2′,5′ or 3′,5′ phosphate linkages.

In some embodiments, the immune cell engager includes 41BB ligand, e.g., comprising the amino acid sequence: ACPWAVSGARASPGSAASPRLREGPELSPDDPAGLLDLRQGMFAQLVAQNVLLIDGPLSWYSDP GLAGVSLTGGLSYKEDTKELVVAKAGVYYVFFQLELRRVVAGEGSGSVSLALHLQPLRSAAGA AALALTVDLPPASSEARNSAFGFQGRLLHLSAGQRLGVHLHTEARARHAWQLTQGATVLGLFR VTPEIPAGLPSPRSE (SEQ ID NO: 3305), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3305.

Toll-Like Receptors: Toll-Like Receptors (TLRs) are evolutionarily conserved receptors are homologues of the Drosophila Toll protein, and recognize highly conserved structural motifs known as pathogen-associated microbial patterns (PAMPs), which are exclusively expressed by microbial pathogens, or danger-associated molecular patterns (DAMPs) that are endogenous molecules released from necrotic or dying cells. PAMPs include various bacterial cell wall components such as lipopolysaccharide (LPS), peptidoglycan (PGN) and lipopeptides, as well as flagellin, bacterial DNA and viral double-stranded RNA. DAMPs include intracellular proteins such as heat shock proteins as well as protein fragments from the extracellular matrix. Stimulation of TLRs by the corresponding PAMPs or DAMPs initiates signaling cascades leading to the activation of transcription factors, such as AP-1, NF-κB and interferon regulatory factors (IRFs). Signaling by TLRs results in a variety of cellular responses, including the production of interferons (IFNs), pro-inflammatory cytokines and effector cytokines that direct the adaptive immune response. TLRs are implicated in a number of inflammatory and immune disorders and play a role in cancer (Rakoff-Nahoum S. & Medzhitov R., 2009. Toll-like receptors and cancer. Nat Revs Cancer 9:57-63).

TLRs are type I transmembrane proteins characterized by an extracellular domain containing leucine-rich repeats (LRRs) and a cytoplasmic tail that contains a conserved region called the Toll/IL-1 receptor (TIR) domain. Ten human and twelve murine TLRs have been characterized, TLR1 to TLR10 in humans, and TLR1 to TLR9, TLR11, TLR12 and TLR13 in mice, the homolog of TLR10 being a pseudogene. TLR2 is essential for the recognition of a variety of PAMPs from Gram-positive bacteria, including bacterial lipoproteins, lipomannans and lipoteichoic acids. TLR3 is implicated in virus-derived double-stranded RNA. TLR4 is predominantly activated by lipopolysaccharide. TLR5 detects bacterial flagellin and TLR9 is required for response to unmethylated CpG DNA. Finally, TLR7 and TLR8 recognize small synthetic antiviral molecules, and single-stranded RNA was reported to be their natural ligand. TLR11 has been reported to recognize uropathogenic E. coli and a profilin-like protein from Toxoplasma gondii. The repertoire of specificities of the TLRs is apparently extended by the ability of TLRs to heterodimerize with one another. For example, dimers of TLR2 and TLR6 are required for responses to diacylated lipoproteins while TLR2 and TLR1 interact to recognize triacylated lipoproteins. Specificities of the TLRs are also influenced by various adapter and accessory molecules, such as MD-2 and CD14 that form a complex with TLR4 in response to LPS.

TLR signaling consists of at least two distinct pathways: a MyD88-dependent pathway that leads to the production of inflammatory cytokines, and a MyD88-independent pathway associated with the stimulation of IFN-β and the maturation of dendritic cells. The MyD88-dependent pathway is common to all TLRs, except TLR3 (Adachi O. et al., 1998. Targeted disruption of the MyD88 gene results in loss of IL-1- and IL-18-mediated function. Immunity. 9(1):143-50). Upon activation by PAMPs or DAMPs, TLRs hetero- or homodimerize inducing the recruitment of adaptor proteins via the cytoplasmic TIR domain. Individual TLRs induce different signaling responses by usage of the different adaptor molecules. TLR4 and TLR2 signaling requires the adaptor TIRAP/Mal, which is involved in the MyD88-dependent pathway. TLR3 triggers the production of IFN-β in response to double-stranded RNA, in a MyD88-independent manner, through the adaptor TRIF/TICAM-1. TRAM/TICAM-2 is another adaptor molecule involved in the MyD88-independent pathway which function is restricted to the TLR4 pathway.

TLR3, TLR7, TLR8 and TLR9 recognize viral nucleic acids and induce type I IFNs. The signaling mechanisms leading to the induction of type I IFNs differ depending on the TLR activated. They involve the interferon regulatory factors. IRFs, a family of transcription factors known to play a critical role in antiviral defense, cell growth and immune regulation. Three IRFs (IRF3, IRF5 and IRF7) function as direct transducers of virus-mediated TLR signaling. TLR3 and TLR4 activate IRF3 and IRF7, while TLR7 and TLR8 activate IRF5 and IRF7 (Doyle S. et al., 2002. IRF3 mediates a TLR3/TLR4-specific antiviral gene program. Immunity. 17(3):251-63). Furthermore, type I IFN production stimulated by TLR9 ligand CpG-A has been shown to be mediated by PI(3)K and mTOR (Costa-Mattioli M. & Sonenberg N. 2008. RAPping production of type I interferon in pDCs through mTOR. Nature Immunol. 9: 1097-1099).

TLR-9: TLR9 recognizes unmethylated CpG sequences in DNA molecules. CpG sites are relatively rare (˜1%) on vertebrate genomes in comparison to bacterial genomes or viral DNA. TLR9 is expressed by numerous cells of the immune system such as B lymphocytes, monocytes, natural killer (NK) cells, and plasmacytoid dendritic cells. TLR9 is expressed intracellularly, within the endosomal compartments and functions to alert the immune system of viral and bacterial infections by binding to DNA rich in CpG motifs. TLR9 signals leads to activation of the cells initiating pro-inflammatory reactions that result in the production of cytokines such as type-I interferon and IL-12.

TLR Agonists: a TLR agonist can agonize one or more TLR, e.g., one or more of human TLR-1, 2, 3, 4, 5, 6, 7, 8, 9, or 10. In some embodiments, an adjunctive agent described herein is a TLR agonist. In some embodiments, the TLR agonist specifically agonizes human TLR-9. In some embodiments, the TLR-9 agonist is a CpG moiety. As used herein, a CpG moiety, is a linear dinucleotide having the sequence: 5′-C-phosphate-G-3′, that is, cytosine and guanine separated by only one phosphate. In some embodiments, the CpG moiety comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more CpG dinucleotides. In some embodiments, the CpG moiety consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 CpG dinucleotides. In some embodiments, the CpG moiety has 1-5, 1-10, 1-20, 1-30, 1-40, 1-50, 5-10, 5-20, 5-30, 10-20, 10-30, 10-40, or 10-50 CpG dinucleotides. In some embodiments, the TLR-9 agonist is a synthetic ODN (oligodeoxynucleotides). CpG ODNs are short synthetic single-stranded DNA molecules containing unmethylated CpG dinucleotides in particular sequence contexts (CpG motifs). CpG ODNs possess a partially or completely phosphorothioated (PS) backbone, as opposed to the natural phosphodiester (PO) backbone found in genomic bacterial DNA. There are three major classes of CpG ODNs: classes A, B and C, which differ in their immunostimulatory activities. CpG-A ODNs are characterized by a PO central CpG-containing palindromic motif and a PS-modified 3′ poly-G string. They induce high IFN-α production from pDCs but are weak stimulators of TLR9-dependent NF-κB signaling and pro-inflammatory cytokine (e.g. IL-6) production. CpG-B ODNs contain a full PS backbone with one or more CpG dinucleotides. They strongly activate B cells and TLR9-dependent NF-κB signaling but weakly stimulate IFN-α secretion. CpG-C ODNs combine features of both classes A and B. They contain a complete PS backbone and a CpG-containing palindromic motif. C-Class CpG ODNs induce strong IFN-α production from pDC as well as B cell stimulation.

### Stromal Modifying Moieties

In some embodiments, the multifunctional molecule further includes a stromal modifying moiety. A “stromal modifying moiety,” as used herein refers to an agent, e.g., a protein (e.g., an enzyme), that is capable of altering, e.g., degrading a component of, the stroma. In embodiments, the component of the stroma is chosen from, e.g., an ECM component, e.g., a glycosaminoglycan, e.g., hyaluronan (also known as hyaluronic acid or HA), chondroitin sulfate, chondroitin, demnatan sulfate, heparin sulfate, heparin, entactin, tenascin, aggrecan and keratin sulfate; or an extracellular protein, e.g., collagen, laminin, elastin, fibrinogen, fibronectin, and vitronectin.

Solid tumors have a distinct structure that mimics that of normal tissues and comprises two distinct but interdependent compartments: the parenchyma (neoplastic cells) and the stroma that the neoplastic cells induce and in which they are dispersed. All tumors have stroma and require stroma for nutritional support and for the removal of waste products. In the case of tumors which grow as cell suspensions (e.g., leukemias, ascites tumors), the blood plasma serves as stroma (Connolly J L et al. Tumor Structure and Tumor Stroma Generation. In: Kufe D W et al., editors. Holland-Frei Cancer Medicine. 6th edition. Hamilton: BC Decker; 2003). The stroma includes a variety of cell types, including fibroblasts/myofibroblasts, glial, epithelial, fat, vascular, smooth muscle, and immune cells along with extracellular matrix (ECM) and extracellular molecules (Li Hanchen et al. Tumor Microenvironment: The Role of the Tumor Stroma in Cancer. J of Cellular Biochemistry 101: 805-815 (2007)).

Stromal modifying moieties described herein include moieties (e.g., proteins, e.g., enzymes) capable of degrading a component of the stroma, e.g., an ECM component, e.g., a glycosaminoglycan, e.g., hyaluronan (also known as hyaluronic acid or HA), chondroitin sulfate, chondroitin, dermatan sulfate, heparin sulfate, heparin, entactin, tenascin, aggrecan and keratin sulfate; or an extracellular protein, e.g., collagen, laminin, elastin, fibrinogen, fibronectin, and vitronectin.

### Stromal Modifying Enzymes

In some embodiments, the stromal modifying moiety is an enzyme. For example, the stromal modifying moiety can include, but is not limited to a hyaluronidase, a collagenase, a chondroitinase, a matrix metalloproteinase (e.g., macrophage metalloelastase).

### Hyaluronidases

Hyaluronidases are a group of neutral- and acid-active enzymes found throughout the animal kingdom. Hyaluronidases vary with respect to substrate specificity, and mechanism of action. There are three general classes of hyaluronidases: (1) Mammalian-type hyaluronidases, (EC 3.2.1.35) which are endo-beta-N-acetylhexosaminidases with tetrasaccharides and hexasaccharides as the major end products. They have both hydrolytic and transglycosidase activities, and can degrade hyaluronan and chondroitin sulfates; (2) Bacterial hyaluronidases (EC 4.2.99.1) degrade hyaluronan and, and to various extents, chondroitin sulfate and dermatan sulfate. They are endo-beta-N-acetylhexosaminidases that operate by a beta elimination reaction that yields primarily disaccharide end products; (3) Hyaluronidases (EC 3.2.1.36) from leeches, other parasites, and crustaceans are endo-beta-glucuronidases that generate tetrasaccharide and hexasaccharide end products through hydrolysis of the beta 1-3 linkage.

Mammalian hyaluronidases can be further divided into two groups: (1) neutral active and (2) acid active enzymes. There are six hyaluronidase-like genes in the human genome, HYAL1, HYAL2, HYAL3 HYAL4 HYALP1 and PH20/SPAM1. HYALP1 is a pseudogene, and HYAL3 has not been shown to possess enzyme activity toward any known substrates. HYAL4 is a chondroitinase and lacks activity towards hyaluronan. HYAL1 is the prototypical acid-active enzyme and PH20 is the prototypical neutral-active enzyme. Acid active hyaluronidases, such as HYAL1 and HYAL2 lack catalytic activity at neutral pH. For example, HYAL1 has no catalytic activity in vitro over pH 4.5 (Frost and Stem, “A Microtiter-Based Assay for Hyaluronidase Activity Not Requiring Specialized Reagents”, Analytical Biochemistry, vol. 251, pp. 263-269 (1997). HYAL2 is an acid active enzyme with a very low specific activity in vitro.

In some embodiments the hyaluronidase is a mammalian hyaluronidase. In some embodiments the hyaluronidase is a recombinant human hyaluronidase. In some embodiments, the hyaluronidase is a neutral active hyaluronidase. In some embodiments, the hyaluronidase is a neutral active soluble hyaluronidase. In some embodiments, the hyaluronidase is a recombinant PH20 neutral-active enzyme. In some embodiments, the hyaluronidase is a recombinant PH20 neutral-active soluble enzyme. In some embodiments the hyaluronidase is glycosylated. In some embodiments, the hyaluronidase possesses at least one N-linked glycan. A recombinant hyaluronidase can be produced using conventional methods known to those of skill in the art, e.g., U.S. Pat. No. 7,767,429, the entire contents of which are incorporated by reference herein.

In some embodiments the hyaluronidase is rHuPH20 (also referred to as Hylenex®; presently manufactured by Halozyme; approved by the FDA in 2005 (see e.g., Scodeller P (2014) Hyaluronidase and other Extracellular Matrix Degrading Enzymes for Cancer Therapy: New Uses and Nano-Formulations. J Carcinog Mutage 5:178; U.S. Pat. Nos. 7,767,429; 8,202,517; 7,431,380; 8,450,470; 8,772,246; 8,580,252, the entire contents of each of which is incorporated by reference herein), rHuPH20 is produced by genetically engineered CHO cells containing a DNA plasmid encoding for a soluble fragment of human hyaluronidase PH20. In some embodiments the hyaluronidase is glycosylated. In some embodiments, the hyaluronidase possesses at least one N-linked glycan. A recombinant hyaluronidase can be produced using conventional methods known to those of skill in the art, e.g., U.S. Pat. No. 7,767,429, the entire contents of which are incorporated by reference herein. In some embodiments, rHuPH20 has a sequence at least 95% (e.g., at least 96%, 97%, 98%, 99%, 100%) identical to the amino acid sequence of

(SEQ ID NO: 3306)
LNFRAPPVIPNVPFLWAWNAPSEFCLGKFDEPLDMSLFSFIGSPRINAT
GQGVTIFYVDRLGYYPYIDSITGVTVNGGIPQKISLQDHLDKAKKDITF
YMPVDNLGMAVIDWEEWRPTWARNWKPKDVYKNRSIELVQQQNVQLSLT
EATEKAKQEFEKAGKDFLVETIKLGKLLRPNHLWGYYLFPDCYNHHYKK
PGYNGSCFNVEIKRNDDLSWLWNESTALYPSIYLNTQQSPVAATLYVRN
RVREAIRVSKIPDAKSPLPVFAYTRIVFTDQVLKFLSQDELVYTFGETV
ALGASGIVIWGTLSIMRSMKSCLLLDNYMETILNPYIINVTLAAKMCSQ
VLCQEQGVCIRKNWNSSDYLHLNPDNFAIQLEKGGKFTVRGKPTLEDLE
QFSEKFYCSCYSTLSCKEKADVKDTDAVDVCIADGVCIDAFLKPPMETE
EPQIFYNASPSTLS.

In any of the methods provided herein, the anti-hyaluronan agent can be an agent that degrades hyaluronan or can be an agent that inhibits the synthesis of hyaluronan. For example, the anti-hyaluronan agent can be a hyaluronan degrading enzyme. In another example, the anti-hyaluronan agent or is an agent that inhibits hyaluronan synthesis. For example, the anti-hyaluronan agent is an agent that inhibits hyaluronan synthesis such as a sense or antisense nucleic acid molecule against an HA synthase or is a small molecule drug. For example, an anti-hyaluronan agent is 4-methylumbelliferone (MU) or a derivative thereof, or leflunomide or a derivative thereof. Such derivatives include, for example, a derivative of 4-methylumbelliferone (MU) that is 6,7-dihydroxy-4-methyl coumarin or 5,7-dihydroxy-4-methyl coumarin.

In further examples of the methods provided herein, the hyaluronan degrading enzyme is a hyaluronidase. In some examples, the hyaluronan-degrading enzyme is a PH20 hyaluronidase or truncated form thereof to lacking a C-terminal glycosylphosphatidylinositol (GPI) attachment site or a portion of the GPI attachment site. In specific examples, the hyaluronidase is a PH20 selected from a human, monkey, bovine, ovine, rat, mouse or guinea pig PH20. For example, the hyaluronan-degrading enzyme is a human PH20 hyaluronidase that is neutral active and N-glycosylated and is selected from among (a) a hyaluronidase polypeptide that is a full-length PH20 or is a C-terminal truncated form of the PH20, wherein the truncated form includes at least amino acid residues 36-464 of SEQ ID NO: 139, such as 36-481, 36-482, 36-483, where the full-length PH20 has the sequence of amino acids set forth in SEQ ID NO: 139; or (b) a hyaluronidase polypeptide comprising a sequence of amino acids having at least 85%, 86%, 87%, 88%, 89%, 90%, 91%, 92%, 93%, 94%, 95%, 96%, 97%, 98%, 99% or more sequence identity with the polypeptide or truncated form of sequence of amino acids set forth in SEQ ID NO: 139; or (c) a hyaluronidase polypeptide of (a) or (b) comprising amino acid substitutions, whereby the hyaluronidase polypeptide has a sequence of amino acids having at least 85%, 86%, 87%, 88%, 89%, 90%, 91%, 92%, 93%, 94% q 95%, 96%, 97%, 98%, 99% or more sequence identity with the polypeptide set forth in SEQ ID NO: 139 or the with the corresponding truncated forms thereof. In exemplary examples, the hyaluronan-degrading enzyme is a PH20 that comprises a composition designated rHuPH20.

In other examples, the anti-hyaluronan agent is a hyaluronan degrading enzyme that is modified by conjugation to a polymer. The polymer can be a PEG and the anti-hyaluronan agent a PEGylated hyaluronan degrading enzyme. Hence, in some examples of the methods provided herein the hyaluronan-degrading enzyme is modified by conjugation to a polymer. For example, the hyaluronan-degrading enzyme is conjugated to a PEG, thus the hyaluronan degrading enzyme is PEGylated. In an exemplary example, the hyaluronan-degrading enzyme is a PEGylated PH20 enzyme (PEGPH20). In the methods provided herein, the corticosteroid can be a glucocorticoid that is selected from among cortisones, dexamethasones, hydrocortisones, methylprednisolones, prednisolones and prednisones.

### Chondroitinases

Chondroitinases are enzymes found throughout the animal kingdom which degrade glycosaminoglycans, specifically chondroitins and chondroitin sulfates, through an endoglycosidase reaction. In some embodiments the chondroitinase is a mammalian chondroitinase. In some embodiments the chondroitinase is a recombinant human chondroitinase. In some embodiments the chondroitinase is HYAL4. Other exemplary chondroitinases include chondroitinase ABC (derived from Proteus vulgaris; Japanese Patent Application Laid-open No 6-153947, T. Yamagata et al. J. Biol. Chem., 243, 1523 (1968), S. Suzuki et al, J. Biol. Chem., 243, 1543 (1968)), chondroitinase AC (derived from Flavobacterium heparinum; T. Yamagata et al., J. Biol. Chem., 243, 1523 (1968)), chondroitinase AC II (derived from Arthrobacter aurescens; K. Hiyama, and S. Okada, J. Biol. Chem., 250, 1824 (1975), K. Hiyama and S. Okada, J. Biochem. (Tokyo), 80, 1201 (1976)), Hyaluronidase ACIII (derived from Flavobacterium sp. Hp102; Hirofumi Miyazono et al., Seikagaku, 61, 1023 (1989)), chondroitinase B (derived from Flavobacterium heparinum; Y. M. Michelacci and C. P. Dietrich, Biochem. Biophys. Res. Commun., 56, 973 (1974), Y. M. Michelacci and C. P. Dietrich, Biochem. J., 151, 121 (1975), Kenichi Maeyama et al, Seikagaku, 57, 1189 (1985)), chondroitinase C (derived from Flavobacterium sp. Hp102; Hirofumi Miyazono et al, Seikagaku, 61, 1023 (1939)), and the like.

### Matrix Metalloproteinases

Matrix metalloproteases (MMPs) are zinc-dependent endopeptidases that are the major proteases involved in extracellular matrix (ECM) degradation. MMPs are capable of degrading a wide range of extracellular molecules and a number of bioactive molecules. Twenty-four MMP genes have been identified in humans, which can be organized into six groups based on domain organization and substrate preference: Collagenases (MMP-1, -8 and -13), Gelatinases (MMP-2 and MMP-9), Stromelysins (MMP-3, -10 and -11), Matrilysin (MMP-7 and MMP-26), Membrane-type (MT)-MMPs (MMP-14, -15, -16, -17, -24 and -25) and others (MMP-12, -19, -20, -21, -23, -27 and -28). In some embodiments, the stromal modifying moiety is a human recombinant MMP (e.g., MMP-1, -2, -3, -4, -5, -6, -7, -8, -9, 10, -11, -12, -13, -14, 15,-15, -17, -18, -19, 20, -21, -22, -23, or -24).

### Collagenases

The three mammalian collagenases (MMP-1, -8, and -13) are the principal secreted endopeptidases capable of cleaving collagenous extracellular matrix. In addition to fibrillar collagens, collagenases can cleave several other matrix and non-matrix proteins including growth factors. Collagenases are synthesized as inactive pro-forms, and once activated, their activity is inhibited by specific tissue inhibitors of metalloproteinases, TIMPs, as well as by non-specific proteinase inhibitors (Ala-aho R et al. Biochimie. Collagenases in cancer. 2005 March-April; 87(3-4):273-86). In some embodiments, the stromal modifying moiety is a collagenase. In some embodiments, the collagenase is a human recombinant collagenase. In some embodiments, the collagenase is MMP-1. In some embodiments, the collagenase is MMP-8. In some embodiments, the collagenase is MMP-13.

### Macrophage Metalloelastase

Macrophage metalloelastase (MME), also known as MMP-12, is a member of the stromelysin subgroup of MMPs and catalyzes the hydrolysis of soluble and insoluble elastin and a broad selection of matrix and nonmatrix substrates including type IV collagen, fibronectin, laminin, vitronectin, entactin, heparan, and chondroitin sulfates (Erja Kerkelä et al. Journal of Investigative Dermatology (2000) 114, 1113-1119; doi:10.1046/j.1523-1747.2000.00993). In some embodiments, the stromal modifying moiety is a MME. In some embodiments, the MME is a human recombinant MME. In some embodiments, the MME is MMP-12.

### Additional Stromal Modifying Moieties

In some embodiments, the stromal modifying moiety causes one or more of: decreases the level or production of a stromal or extracellular matrix (ECM) component; decreases tumor fibrosis; increases interstitial tumor transport, improves tumor perfusion; expands the tumor microvasculature; decreases interstitial fluid pressure (IFP) in a tumor; or decreases or enhances penetration or diffusion of an agent, e.g., a cancer therapeutic or a cellular therapy, into a tumor or tumor vasculature.

In some embodiments, the stromal or ECM component decreased is chosen from a glycosaminoglycan or an extracellular protein, or a combination thereof. In some embodiments, the glycosaminoglycan is chosen from hyaluronan (also known as hyaluronic acid or HA), chondroitin sulfate, chondroitin, dermatan sulfate, heparin, heparin sulfate, entactin, tenascin, aggrecan and keratin sulfate. In some embodiments, the extracellular protein is chosen from collagen, laminin, elastin, fibrinogen, fibronectin, or vitronectin. In some embodiments, the stromal modifying moiety includes an enzyme molecule that degrades a tumor stroma or extracellular matrix (ECM). In some embodiments, the enzyme molecule is chosen from a hyaluronidase molecule, a collagenase molecule, a chondroitinase molecule, a matrix metalloproteinase molecule (e.g., macrophage metalloelastase), or a variant (e.g., a fragment) of any of the aforesaid. The term “enzyme molecule” includes a full length, a fragment or a variant of the enzyme, e.g., an enzyme variant that retains at least one functional property of the naturally-occurring enzyme.

In some embodiments, the stromal modifying moiety decreases the level or production of hyaluronic acid. In other embodiments, the stromal modifying moiety comprises a hyaluronan degrading enzyme, an agent that inhibits hyaluronan synthesis, or an antibody molecule against hyaluronic acid.

In some embodiments, the hyaluronan degrading enzyme is a hyaluronidase molecule, e.g., a full length or a variant (e.g., fragment thereof) thereof. In some embodiments, the hyaluronan degrading enzyme is active in neutral or acidic pH, e.g., pH of about 4-5. In some embodiments, the hyaluronidase molecule is a mammalian hyaluronidase molecule, e.g., a recombinant human hyaluronidase molecule, e.g., a full length or a variant (e.g., fragment thereof, e.g., a truncated form) thereof. In some embodiments, the hyaluronidase molecule is chosen from HYAL1, HYAL2, or PH-20/SPAM1, or a functional fragment or a functional variant thereof (e.g., a truncated form thereof). In some embodiments, the truncated form lacks a C-terminal glycosylphosphatidylinositol (GPI) attachment site or a portion of the GPI attachment site. In some embodiments, the hyaluronidase molecule is glycosylated, e.g., comprises at least one N-linked glycan.

In some embodiments, the hyaluronidase molecule comprises the amino acid sequence: LNFRAPPVIPNVPFLWAWNAPSEFCLGKFDEPLDMSLFSFIGSPRINATGQGVTIFYVDRLGYYPY IDSITGVTVNGGIPQKISLQDHLDKAKKDITFYMPVDNLGMAVIDWEEWRPTWARNWKPKDVY KNRSIELVQQQNVQLSLTEATEKAKQEFEKAGKDFLVETIKLGKLLRPNHLWGYYLFPDCYNHH YKKPGYNGSCFNVEIKRNDDLSWLWNESTALYPSIYLNTQQSPVAATLYVRNRVREAIRVSKIPD AKSPLPVFAYTRIVFTDQVLKFLSQDELVYTFGETVALGASGIVIWGTLSIMRSMKSCLLLDNYM ETILNPYIINVTLAAKMCSQVLCQEQGVCIRKNWNSSDYLHLNPDNFAIQLEKGGKFTVRGKPTL EDLEQFSEKFYCSCYSTLSCKEKADVKDTDAVDVCIADGVCIDAFLKPPMETEEPQIFYNASPSTL S (SEQ ID NO:3311), or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3311.

In some embodiments, the hyaluronidase molecule comprises: (i) the amino acid sequence of 36-464 of SEQ ID NO: 3311; (ii) the amino acid sequence of 36-481, 36-482, or 36-483 of PH20, wherein PH20 has the sequence of amino acids set forth in SEQ ID NO: 3311; or (iii) an amino acid sequence having at least 95% to 100% sequence identity to the polypeptide or truncated form of sequence of amino acids set forth in SEQ ID NO: 3311; or (iv) an amino acid sequence having 30, 20, 10, 5 or fewer amino acid substitutions to the amino acid sequence set forth in SEQ ID NO: 3311. In some embodiments, the hyaluronidase molecule comprises an amino acid sequence at least 95% (e.g., at least 95%, 96%, 97%, 98%, 99%, 100%) identical to the amino acid sequence of SEQ ID NO: 3311. In some embodiments, the hyaluronidase molecule is encoded by a nucleotide sequence at least 95% (e.g., at least 96%, 97%, 98%, 99%, 100%) identical to the nucleotide sequence of SEQ ID NO: 3311.

In some embodiments, the hyaluronidase molecule is PH20, e.g., rHuPH20. In some embodiments, the hyaluronidase molecule is HYAL1 and comprises the amino acid sequence: FRGPLLPNRPFTTVWNANTQWCLERHGVDVDVSVFDVVANPGQTFRGPDMTIFYSSQGTYPYY TPTGEPVFGGLPQNASLIAHLARTFQDILAAIPAPDFSGLAVIDWEAWRPRWAFNWDTKDIYRQR SRALVQAQHPDWPAPQVEAVAQDQFQGAARAWMAGTLQLGRALRPRGLWGFYGFPDCYNYD FLSPNYTGQCPSGIRAQNDQLGWLWGQSRALYPSIYMPAVLEGTGKSQMYVQHRVAEAFRVAV AAGDPNLPVLPYVQIFYDTTNHFLPLDELEHSLGESAAQGAAGVVLWVSWENTRTKESCQAIKE YMDTTLGPFILNVTSGALLCSQALCSGHGRCVRRTSHPKALLLLNPA SFSIQLTPGGGPLSLRGAL SLEDQAQMAVEFKCRCYPGWQAPWCERKSMW (SEQ ID NO: 3312), or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3312.

In some embodiments, the hyaluronan degrading enzyme, e.g., the hyaluronidase molecule, further comprises a polymer, e.g., is conjugated to a polymer, e.g., PEG. In some embodiments, the hyaluronan-degrading enzyme is a PEGylated PH20 enzyme (PEGPH20). In some embodiments, the hyaluronan degrading enzyme, e.g., the hyaluronidase molecule, further comprises an immunoglobulin chain constant region (e.g., Fc region) chosen from, e.g., the heavy chain constant regions of IgG1, IgG2, IgG3, and IgG4, more particularly, the heavy chain constant region of human IgG1, IgG2, IgG3, or IgG4. In some embodiments, the immunoglobulin constant region (e.g., the Fc region) is linked, e.g., covalently linked to, the hyaluronan degrading enzyme, e.g., the hyaluronidase molecule. In some embodiments, the immunoglobulin chain constant region (e.g., Fc region) is altered, e.g., mutated, to increase or decrease one or more of: Fc receptor binding, antibody glycosylation, the number of cysteine residues, effector cell function, or complement function. In some embodiments, the hyaluronan degrading enzyme, e.g., the hyaluronidase molecule forms a dimer.

In some embodiments, the stromal modifying moiety comprises an inhibitor of the synthesis of hyaluronan, e.g., an HA synthase. In some embodiments, the inhibitor comprises a sense or an antisense nucleic acid molecule against an HA synthase or is a small molecule drug. In some embodiments, the inhibitor is 4-methylumbelliferone (MU) or a derivative thereof (e.g., 6,7-dihydroxy-4-methyl coumarin or 5,7-dihydroxy-4-methyl coumarin), or leflunomide or a derivative thereof.

In some embodiments, the stromal modifying moiety comprises antibody molecule against hyaluronic acid.

In some embodiments, the stromal modifying moiety comprises a collagenase molecule, e.g., a mammalian collagenase molecule, or a variant (e.g., fragment) thereof. In some embodiments, the collagenase molecule is collagenase molecule IV, e.g., comprising the amino acid sequence of: YNFFPRKPKWDKNQITYRIIGYTPDLDPETVDDAFARAFQVWSDVTPLRFSRIHDGEADIMINFG RWEHGDGYPFDGKDGLLAHAFAPGTGVGGDSHFDDDELWTLGEGQVVRVKYGNADGEYCKF PFLFNGKEYNSCTDTGRSDGFLWCSTTYNFEKDGKYGFCPHEALFTMGGNAEGQPCKFPFRFQG TSYDSCTTEGRTDGYRWCGTTEDYDRDKKYGFCPETAMSTVGGNSEGAPCVFPFTFLGNKYESC TSAGRSDGKMWCATTANYDDDRKWGFCPDQGYSLFLVAAHEFGHAMGLEHSQDPGALMAPIY TYTKNFRLSQDDIKGIQELYGASPDIDLGTGPTPTLGPVTPEICKQDIVFDGIAQIRGEIFFFKDRFI WRTVTPRDKPMGPLLVATFWPELPEKIDAVYEAPQEEKAVFFAGNEYWIYSASTLERGYPKPLT SLGLPPDVQRVDAAFNWSKNKKTYIFAGDKFWRYNEVKKKMDPGFPKLIADAWNAIPDNLDA VVDLQGGGHSYFFKGAYYLKLENQSLKSVKFGSIKSDWLGC (SEQ ID NO: 3313), or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3313.

### Tumor Antigen Moiety

In some embodiments, the multifunctional molecule further includes a tumor antigen moiety. In some embodiments, the tumor-targeting moiety is an antigen, e.g., a cancer antigen. In some embodiments, the cancer antigen is a tumor antigen or stromal antigen, or a hematological antigen.

“Cancer” as used herein can encompass all types of oncogenic processes and/or cancerous growths. In embodiments, cancer includes primary tumors as well as metastatic tissues or malignantly transformed cells, tissues, or organs. In embodiments, cancer encompasses all histopathologies and stages, e.g., stages of invasiveness/severity, of a cancer. In embodiments, cancer includes relapsed and/or resistant cancer. The terms “cancer” and “tumor” can be used interchangeably. For example, both terms encompass solid and liquid tumors. As used herein, the term “cancer” or “tumor” includes premalignant, as well as malignant cancers and tumors. As used herein, the terms “tumor antigen”, “tumor-associated antigen”, and “tumor associate antigen” can be used interchangeably.

In some embodiments, the tumor-targeting moiety, e.g., cancer antigen, is chosen from: BCMA, FcRH5, CD19, CD20, CD22, CD30, CD33, CD38, CD47, CD99, CD123, FcRH5, CLEC12, CD179A, SLAMF7, or NY-ESO1, PDL1, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDC27, a actinin-4, TRP1/gp75, TRP2, gp100, Melan-A/MART1, gangliosides, WT1, EphA3, Epidermal growth factor receptor (EGFR), MART-2, MART-1, MUC1, MUC2, MUM1, MUM2, MUM3, NA88-1, NPM, OA1, OGT, RCC, RUI1, RUI2, SAGE, TRG, TRP1, TSTA, Folate receptor alpha, L1-CAM, CAIX, gpA33, GD3, GM2, VEGFR, Integrins (Integrin alphaVbeta3, Integrin alpha5Beta1), Carbohydrates (Le), IGF1R, EPHA3, TRAILR1, TRAILR2, RANKL, (FAP), TGF-beta, hyaluronic acid, collagen, e.g., collagen IV, tenascin C, or tenascin W. In some embodiments, the tumor-targeting moiety, e.g., cancer antigen, is BCMA. In some embodiments, the tumor-targeting moiety, e.g., cancer antigen, is FcRH5.

In some embodiments, the tumor-targeting moiety binds to a cancer antigen chosen from: CD19, CD123, CD22, CD30, CD171, CS-1, C-type lectin-like molecule-1, CD33, epidermal growth factor receptor variant III (EGFRvIII), ganglioside G2 (GD2), ganglioside GD3, TNF receptor family member B cell maturation (BCMA). Tn antigen ((Tn Ag) or (GalNAcα-Ser/Thr)), prostate-specific membrane antigen (PSMA), Receptor tyrosine kinase-like orphan receptor 1 (ROR1), Fms-Like Tyrosine Kinase 3 (FLT3), Tumor-associated glycoprotein 72 (TAG72), CD38, CD44v6, Carcinoembryonic antigen (CEA), Epithelial cell adhesion molecule (EPCAM), B7H3 (CD276). KIT (CD117), Interleukin-13 receptor subunit alpha-2, mesothelin, Interleukin 11 receptor alpha (IL-11Ra), prostate stem cell antigen (PSCA), Protease Serine 21, vascular endothelial growth factor receptor 2 (VEGFR2), Lewis(Y) antigen, CD24, Platelet-derived growth factor receptor beta (PDGFR-beta), Stage-specific embryonic antigen-4 (SSEA-4), CD20, Folate receptor alpha, Receptor tyrosine-protein kinase ERBB2 (Her2/neu), Mucin 1, cell surface associated (MUC1), epidermal growth factor receptor (EGFR), neural cell adhesion molecule (NCAM). Prostase, prostatic acid phosphatase (PAP), elongation factor 2 mutated (ELF2M), Ephrin B2, fibroblast activation protein alpha (FAP), insulin-like growth factor 1 receptor (IGF-1 receptor), carbonic anhydrase IX (CAIX), Proteasome (Prosome, Macropain) Subunit, Beta Type, 9 (LMP2), glycoprotein 100 (gp100), oncogene fusion protein consisting of breakpoint cluster region (BCR) and Abelson murine leukemia viral oncogene homolog 1 (Abl) (bcr-abl), tyrosinase, ephrin type-A receptor 2 (EphA2), Fucosyl GM1, sialyl Lewis adhesion molecule (sLe), ganglioside GM3, transglutaminase 5 (TGS5), high molecular weight-melanoma-associated antigen (HMWMAA), o-acetyl-GD2 ganglioside (OAcGD2), Folate receptor beta, tumor endothelial marker 1 (TEM1/CD248), tumor endothelial marker 7-related (TEM7R), claudin 6 (CLDN6), thyroid stimulating hormone receptor (TSHR), G protein-coupled receptor class C group 5, member D (GPRC5D), chromosome X open reading frame 61 (CXORF61), CD97, CD179a, anaplastic lymphoma kinase (ALK), Polysialic acid, placenta-specific 1 (PLAC1), hexasaccharide portion of globoH glycoceramide (GloboH), mammary gland differentiation antigen (NY-BR-1), uroplakin 2 (UPK2), Hepatitis A virus cellular receptor 1 (HAVCR1), adrenoceptor beta 3 (ADRB3), pannexin 3 (PANX3), G protein-coupled receptor 20 (GPR20), lymphocyte antigen 6 complex, locus K 9 (LY6K), Olfactory receptor 51E2 (OR51E2), TCR Gamma Alternate Reading Frame Protein (TARP), Wilms tumor protein (WT1), Cancer/testis antigen 1 (NY-ESO-1), Cancer/testis antigen 2 (LAGE-1a), Melanoma-associated antigen 1 (MAGE-A1), ETS translocation-variant gene 6, located on chromosome 12p (ETV6-AML), sperm protein 17 (SPA17), X Antigen Family, Member 1A (XAGE1), angiopoietin-binding cell surface receptor 2 (Tie 2), melanoma cancer testis antigen-1 (MAD-CT-1), melanoma cancer testis antigen-2 (MAD-CT-2), Fos-related antigen 1, tumor protein p53 (p53), p53 mutant, prostein, surviving, telomerase, prostate carcinoma tumor antigen-1, melanoma antigen recognized by T cells 1, Rat sarcoma (Ras) mutant, human Telomerase reverse transcriptase (hTERT), sarcoma translocation breakpoints, melanoma inhibitor of apoptosis (ML-IAP), ERG (transmembrane protease, serine 2 (TMPRSS2) ETS fusion gene), N-Acetyl glucosaminyl-transferase V (NA17), paired box protein Pax-3 (PAX3), Androgen receptor, Cyclin B1, v-myc avian myelocytomatosis viral oncogene neuroblastoma derived homolog (MYCN), Ras Homolog Family Member C (RhoC), Tyrosinase-related protein 2 (TRP-2), Cytochrome P450 1B1 (CYP1B1), CCCTC-Binding Factor (Zinc Finger Protein)-Like, Squamous Cell Carcinoma Antigen Recognized By T Cells 3 (SART3), Paired box protein Pax-5 (PAX5), proacrosin binding protein sp32 (OY-TES1), lymphocyte-specific protein tyrosine kinase (LCK), A kinase anchor protein 4 (AKAP-4), synovial sarcoma, X breakpoint 2 (SSX2), Receptor for Advanced Glycation Endproducts (RAGE-1), renal ubiquitous 1 (RU1), renal ubiquitous 2 (RU2), legumain, human papilloma virus E6 (HPV E6), human papilloma virus E7 (HPV E7), intestinal carboxyl esterase, heat shock protein 70-2 mutated (mut hsp70-2), CD79a, CD79b, CD72, Leukocyte-associated immunoglobulin-like receptor 1 (LAIR1), Fc fragment of IgA receptor (FCAR or CD89), Leukocyte immunoglobulin-like receptor subfamily A member 2 (LILRA2), CD300 molecule-like family member f (CD300LF), C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), or immunoglobulin lambda-like polypeptide 1 (IGLL1).

### FcRH5 Targeting Moieties:

In some embodiments, the multispecific molecules as described herein include a targeting moiety that binds to FcRH5 (e.g., a FcRH5 targeting moiety). The FcRH5 targeting moiety can be chosen from an antibody molecule (e.g., an antigen binding domain as described herein), a receptor or a receptor fragment, or a ligand or a ligand fragment, or a combination thereof. In some embodiments, the FcRH5 targeting moiety associates with, e.g., binds to, a cancer or hematopoietic cell (e.g., a molecule, e.g., antigen, present on the surface of the cancer or hematopoietic cell). In certain embodiments, the FcRH5 targeting moiety targets, e.g., directs the multispecific molecules as described herein to a cancer or hematopoietic cell. In some embodiments, the cancer is a hematological cancer, e.g., multiple myeloma.

In some embodiments, the multispecific molecule, e.g., the FcRH5 targeting moiety, binds to a FcRH5 antigen on the surface of a cell, e.g., a cancer or hematopoietic cell. The FcRH5 antigen can be present on a primary tumor cell, or a metastatic lesion thereof. In some embodiments, the cancer is a hematological cancer, e.g., multiple myeloma. For example, the FcRH5 antigen can be present on a tumor, e.g., a tumor of a class typified by having one or more of: limited tumor perfusion, compressed blood vessels, or fibrotic tumor interstitium.

The multispecific molecules described herein includes a FcRH5 targeting moiety that comprises an anti-FcRH5 antibody or antigen-binding fragment thereof described in U.S. Pat. No. 7,999,077, US20150098900, U.S. Pat. Nos. 8,299,220, 7,105,149, 8,362,213, 8,466,260, 8,617,559, US20160368985, US20150166661, and US20080247944, the entire contents of any of the aforesaid publications are herein incorporated by reference.

In some embodiments, the multispecific molecules described herein includes a FcRH5 targeting moiety that comprises an anti-FcRH5 antibody or antigen-binding fragment thereof described in U.S. Pat. No. 7,999,077, the entire contents of which are herein incorporated by reference.

### BCMA Targeting Moieties:

In certain embodiments, the multispecific molecules as described herein include a targeting moiety that binds to BCMA (e.g., a BCMA targeting moiety). The BCMA targeting moiety can be chosen from an antibody molecule (e.g., an antigen binding domain as described herein), a receptor or a receptor fragment, or a ligand or a ligand fragment, or a combination thereof. In some embodiments, the BCMA targeting moiety associates with, e.g., binds to, a cancer or hematopoietic cell (e.g., a molecule, e.g., antigen, present on the surface of the cancer or hematopoietic cell). In certain embodiments, the BCMA targeting moiety targets, e.g., directs the multispecific molecules as described herein to a cancer or hematopoietic cell. In some embodiments, the cancer is a hematological cancer, e.g., multiple myeloma.

In some embodiments, the multispecific molecule, e.g., the BCMA targeting moiety, binds to a BCMA antigen on the surface of a cell, e.g., a cancer or hematopoietic cell. The BCMA antigen can be present on a primary tumor cell, or a metastatic lesion thereof. In some embodiments, the cancer is a hematological cancer, e.g., multiple myeloma. For example, the BCMA antigen can be present on a tumor, e.g., a tumor of a class typified by having one or more of: limited tumor perfusion, compressed blood vessels, or fibrotic tumor interstitium.

Exemplary BCMA targeting moieties: the multispecific molecules described herein can include a BCMA targeting moiety that comprises an anti-BCMA antibody or antigen-binding fragment thereof described in U.S. Pat. Nos. 8,920,776, 9,243,058, 9,340,621, 8,846,042, 7,083,785, 9,545,086, 7,276,241, 9,034,324, 7,799,902, 9,387,237, 8,821,883, US861745, US20130273055, US20160176973, US20150368351. US20150376287, US20170022284, US20160015749, US20140242077, US20170037128, US20170051068, US20160368988, US20160311915, US20160131654, US20120213768, US20110177093, US20160297885, EP3137500, EP2699259, EP2982694, EP3029068, EP3023437, WO2016090327, WO2017021450, WO2016110584, WO2016118641, WO2016168149, the entire contents of which are incorporated herein by reference.

In some embodiments, the BCMA-targeting moiety includes an antibody molecule (e.g., Fab or scFv) that binds to BCMA. In some embodiments, the antibody molecule to BCMA comprises one, two, or three CDRs from any of the heavy chain variable domain sequences of Table 1, or a closely related CDR, e.g., CDRs which have at least one amino acid alteration, but not more than two, three or four alterations (e.g., substitutions, deletions, or insertions, e.g., conservative substitutions) from any of the CDR sequences of Table 15. In some embodiments, the antibody molecule to BCMA comprises a heavy chain variable domain sequence chosen from any of the amino acid sequences of Table 15, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions)).

Alternatively, or in combination with the heavy chain to BCMA as described herein, the antibody molecule to BCMA comprises one, two, or three CDRs from any of the light chain variable domain sequences of Table 15, or a closely related CDR, e.g., CDRs which have at least one amino acid alteration, but not more than two, three or four alterations (e.g., substitutions, deletions, or insertions, e.g., conservative substitutions) from any of the CDR sequences of Table 15. In some embodiments, the antibody molecule to BCMA comprises a light chain variable domain sequence chosen from any of the amino acid sequences of Table 15, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions)).

### Tumor-Targeting Moieties

In some embodiments, the multifunctional or multispecific (e.g., bi-, tri-, tetra-specific) molecules as described herein further include, e.g., are engineered to further contain, one or more tumor specific targeting moieties that direct the molecule to a tumor cell.

In certain embodiments, the multispecific molecules as described herein further include a tumor-targeting moiety. The tumor targeting moiety can be chosen from an antibody molecule (e.g., an antigen binding domain as described herein), a receptor or a receptor fragment, or a ligand or a ligand fragment, or a combination thereof. In some embodiments, the tumor targeting moiety associates with, e.g., binds to, a tumor cell (e.g., a molecule, e.g., antigen, present on the surface of the tumor cell). In certain embodiments, the tumor targeting moiety targets, e.g., directs the multispecific molecules as described herein to a cancer (e.g., a cancer or tumor cells). In some embodiments, the cancer is chosen from a hematological cancer, a solid cancer, a metastatic cancer, or a combination thereof.

In some embodiments, the multispecific molecule, e.g., the tumor-targeting moiety, binds to a solid tumor antigen or a stromal antigen. The solid tumor antigen or stromal antigen can be present on a solid tumor, or a metastatic lesion thereof. In some embodiments, the solid tumor is chosen from one or more of pancreatic (e.g., pancreatic adenocarcinoma), breast, colorectal, lung (e.g., small or non-small cell lung cancer), skin, ovarian, or liver cancer. In some embodiments, the solid tumor is a fibrotic or desmoplastic solid tumor. For example, the solid tumor antigen or stromal antigen can be present on a tumor, e.g., a tumor of a class typified by having one or more of: limited tumor perfusion, compressed blood vessels, or fibrotic tumor interstitium.

In certain embodiments, the solid tumor antigen is chosen from one or more of: PDL1. CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDC27, CD47, a actinin-4, TRP1/gp75, TRP2, gp100, Melan-A/MART1, gangliosides, WT1, EphA3, Epidermal growth factor receptor (EGFR), MART-2, MART-1, MUC1, MUC2, MUM1, MUM2, MUM3, NA88-1, NPM, OA1, OGT, RCC, RUI1, RUI2, SAGE, TRG, TRP1, TSTA, Folate receptor alpha, Li-CAM, CAIX, EGFRvIII, gpA33, GD3, GM2, VEGFR, Intergrins (Integrin alphaVbeta3, Integrin alpha5Beta1), Carbohydrates (Le), IGF1R, EPHA3, TRAILR1, TRAILR2, or RANKL.

In other embodiments, the multispecific molecule, e.g., the tumor-targeting moiety, binds to a molecule, e.g., antigen, present on the surface of a hematological cancer, e.g., a leukemia or a lymphoma. In some embodiments, the hematological cancer is a B-cell or T cell malignancy. In some embodiments, the hematological cancer is chosen from one or more of a Hodgkin's lymphoma, Non-Hodgkin's lymphoma (e.g., B cell lymphoma, diffuse large B cell lymphoma, follicular lymphoma, chronic lymphocytic leukemia, mantle cell lymphoma, marginal zone B-cell lymphoma, Burkitt lymphoma, lymphoplasmacytic lymphoma, hairy cell leukemia), acute myeloid leukemia (AML), chronic myeloid leukemia, myelodysplastic syndrome (MDS), multiple myeloma, or acute lymphocytic leukemia. In embodiments, the cancer is other than acute myeloid leukemia (AML) or myelodysplastic syndrome (MDS). In embodiments, the hematological antigen is chosen from CD47, CD99, CD30, CD38, SLAMF7, or NY-ESO1. In some embodiments, the hematological antigen is chosen from is chosen from one or more of: BCMA, CD19, CD20, CD22, CD33, CD123, FcRH5, CLEC12, or CD179A.

In some embodiments, the tumor-associated antigen-binding moiety comprises an antibody or an antigen-binding moiety thereof that binds to CD19, CD123, CD22, CD30, CD171, CS-1, C-type lectin-like molecule-1, CD33, CISH, epidermal growth factor receptor variant III (EGFRvIII), ganglioside G2 (GD2), ganglioside GD3, TNF receptor family member B cell maturation (BCMA), Tn antigen, prostate-specific membrane antigen (PSMA), Receptor tyrosine kinase-like orphan receptor 1 (ROR1), Fms-Like Tyrosine Kinase 3 (FLT3), Tumor-associated glycoprotein 72 (TAG72), CD38, CD44v6, Carcinoembryonic antigen (CEA), Epithelial cell adhesion molecule (EPCAM), B7H3 (CD276), KIT (CD117), Interleukin-13 receptor subunit alpha-2, mesothelin, Interleukin 11 receptor alpha (IL-11Ra), prostate stem cell antigen (PSCA), Protease Serine 21, vascular endothelial growth factor receptor 2 (VEGFR2), Lewis(Y) antigen, CD24, Platelet-derived growth factor receptor beta (PDGFR-beta). Stage-specific embryonic antigen-4 (SSEA-4), CD20, Folate receptor alpha, Receptor tyrosine-protein kinase ERBB2 (Her2/neu), Mucin 1, cell surface associated (MUC1), epidermal growth factor receptor (EGFR), neural cell adhesion molecule (NCAM), Prostase, prostatic acid phosphatase (PAP), elongation factor 2 mutated (ELF2M), Ephrin B2, fibroblast activation protein alpha (FAP), insulin-like growth factor 1 receptor (IGF-I receptor), carbonic anhydrase IX (CAIX), Proteasome (Prosome, Macropain) Subunit, Beta Type, 9 (LMP2), glycoprotein 100 (gp100), oncogene fusion protein consisting of breakpoint cluster region (BCR) and Abelson murine leukemia viral oncogene homolog 1 (Abl) (bcr-abl), tyrosinase, ephrin type-A receptor 2 (EphA2), Fucosyl GM1, sialyl Lewis adhesion molecule (sLe), ganglioside GM3, transglutaminase 5 (TGS5), high molecular weight-melanoma-associated antigen (HMWMAA), o-acetyl-GD2 ganglioside (OAcGD2), Folate receptor beta, tumor endothelial marker 1 (TEM1/CD248), tumor endothelial marker 7-related (TEM7R), claudin 6 (CLDN6), thyroid stimulating hormone receptor (TSHR), G protein-coupled receptor class C group 5, member D (GPRC5D), chromosome X open reading frame 61 (CXORF61), CD97, CD179a, anaplastic lymphoma kinase (ALK). Polysialic acid, placenta-specific 1 (PLAC1), hexasaccharide portion of globoH glycoceramide (GloboH), mammary gland differentiation antigen (NY-BR-1), uroplakin 2 (UPK2), Hepatitis A virus cellular receptor 1 (HAVCR1), adrenoceptor beta 3 (ADRB3), pannexin 3 (PANX3), G protein-coupled receptor 20 (GPR20), lymphocyte antigen 6 complex, locus K 9 (LY6K), Olfactory receptor 51E2 (OR51E2), TCR Gamma Alternate Reading Frame Protein (TARP), Wilms tumor protein (WT1), Cancer/testis antigen 1 (NY-ESO-1), Cancer/testis antigen 2 (LAGE-1a), Melanoma-associated antigen 1 (MAGE-A1), ETS translocation-variant gene 6, located on chromosome 2p (ETV6-AML), sperm protein 17 (SPA17), X Antigen Family, Member 1A (XAGE1), angiopoietin-binding cell surface receptor 2 (Tie 2), melanoma cancer testis antigen-1 (MAD-CT-1), melanoma cancer testis antigen-2 (MAD-CT-2), Fos-related antigen 1, tumor protein p53 (p53), p53 mutant prostein, survivin, telomerase, prostate carcinoma tumor antigen-1, melanoma antigen recognized by T cells 1, Rat sarcoma (Ras) mutant, human Telomerase reverse transcriptase (hTERT), sarcoma translocation breakpoints, melanoma inhibitor of apoptosis (ML-IAP), ERG (transmembrane protease, serine 2 (TMPRSS2) ETS fusion gene), N-Acetyl glucosaminyl-transferase V (NA17), paired box protein Pax-3 (PAX3), Androgen receptor, Cyclin B1, v-myc avian myelocytomatosis viral oncogene neuroblastoma derived homolog (MYCN), Ras Homolog Family Member C (RhoC), Tyrosinase-related protein 2 (TRP-2), Cytochrome P450 1B1 (CYP1B1), CCCTC-Binding Factor (Zinc Finger Protein)-Like, Squamous Cell Carcinoma Antigen Recognized By T Cells 3 (SART3), Paired box protein Pax-5 (PAX5), proacrosin binding protein sp32 (OY-TES1), lymphocyte-specific protein tyrosine kinase (LCK), A kinase anchor protein 4 (AKAP-4), synovial sarcoma, X breakpoint 2 (SSX2), Receptor for Advanced Glycation Endproducts (RAGE-1), renal ubiquitous 1 (RU1), renal ubiquitous 2 (RU2), legumain, human papilloma virus E6 (HPV E6), human papilloma virus E7 (HPV E7), intestinal carboxyl esterase, heat shock protein 70-2 mutated (mut hsp70-2), CD79a, CD79b, CD72, Leukocyte-associated immunoglobulin-like receptor 1 (LAIR1), Fc fragment of IgA receptor (FCAR or CD89), Leukocyte immunoglobulin-like receptor subfamily A member 2 (LILRA2), CD300 molecule-like family member f (CD300LF). C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), or immunoglobulin lambda-like polypeptide 1 (IGLL1) antigen.

### Antibody Molecules

In some embodiments, the antibody molecule binds to a cancer antigen, e.g., a tumor antigen or a stromal antigen. In some embodiments, the cancer antigen is, e.g., a mammalian, e.g., a human, cancer antigen. In other embodiments, the antibody molecule binds to an immune cell antigen, e.g., a mammalian, e.g., a human, immune cell antigen. For example, the antibody molecule binds specifically to an epitope, e.g., linear or conformational epitope, on the cancer antigen or the immune cell antigen.

In some embodiments, an antibody molecule is a monospecific antibody molecule and binds a single epitope. E.g., a monospecific antibody molecule having a plurality of immunoglobulin variable domain sequences, each of which binds the same epitope.

In some embodiments, an antibody molecule is a multispecific or multifunctional antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein). In some embodiments, a multispecific antibody molecule comprises a third, fourth or fifth immunoglobulin variable domain. In some embodiments, a multispecific antibody molecule is a bispecific antibody molecule, a trispecific antibody molecule, or a tetraspecific antibody molecule.

In some embodiments, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein). In some embodiments, a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In some embodiments, a bispecific antibody molecule comprises a half antibody having binding specificity for a first epitope and a half antibody having binding specificity for a second epitope. In some embodiments, a bispecific antibody molecule comprises a half antibody, or fragment thereof, having binding specificity for a first epitope and a half antibody, or fragment thereof, having binding specificity for a second epitope. In some embodiments, a bispecific antibody molecule comprises a scFv or a Fab, or fragment thereof, have binding specificity for a first epitope and a scFv or a Fab, or fragment thereof, have binding specificity for a second epitope.

In some embodiments, an antibody molecule comprises a diabody, and a single-chain molecule, as well as an antigen-binding fragment of an antibody (e.g., Fab, F(ab′)₂, and Fv). For example, an antibody molecule can include a heavy (H) chain variable domain sequence (abbreviated herein as VH), and a light (L) chain variable domain sequence (abbreviated herein as VL). In some embodiments, an antibody molecule comprises or consists of a heavy chain and a light chain (referred to herein as a half antibody. In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The preparation of antibody molecules can be monoclonal or polyclonal. An antibody molecule can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

Examples of antigen-binding fragments of an antibody molecule include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments are obtained using conventional techniques known to those with skill in the art, and the fragments are screened for utility in the same manner as are intact antibodies.

Antibody molecules include intact molecules as well as functional fragments thereof. Constant regions of the antibody molecules can be altered, e.g., mutated, to modify the properties of the antibody (e.g., to increase or decrease one or more of: Fc receptor binding, antibody glycosylation, the number of cysteine residues, effector cell function, or complement function).

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition. U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann. R., Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia, the CDR amino acids in the VH are numbered 26-32(HCDR1), 52-56 (HCDR2), and 95-102 (HCDR3); and the amino acid residues in VL are numbered 26-32 (LCDR1), 50-52 (LCDR2), and 91-96 (LCDR3).

Each VH and VL typically includes three CDRs and four FRs, arranged from amino-terminus to carboxy-terminus in the following order: FR1, CDR1, FR2, CDR2, FR3, CDR3, FR4.

The antibody molecule can be a polyclonal or a monoclonal antibody.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

The antibody can be recombinantly produced, e.g., produced by phage display or by combinatorial methods, or by yeast display.

Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio-Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

The yeast display method for generating or identifying antibodies is known in the art, e.g., as described in Chao et al. (2006) Nature Protocols 1(2):755-68, the entire contents of which is incorporated by reference herein.

In some embodiments, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993PNAS 90:3720-3724; Bruggeman et al. 1991 Eur J Immunol 21:1323-1326).

An antibody molecule can be one in which the variable region, or a portion thereof, e.g., the CDRs, are generated in a non-human organism, e.g., a rat or mouse. Chimeric, CDR-grafted, and humanized antibodies are within the invention. Antibody molecules generated in a non-human organism, e.g., a rat or mouse, and then modified, e.g., in the variable framework or constant region, to decrease antigenicity in a human are within the invention.

An “effectively human” protein is a protein that does substantially not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see. e.g., Saleh et al., Cancer Immunol. Immunother., 32:180-190 (1990)) and also because of potential allergic reactions (see. e.g., LoBuglio et al., Hybridoma, 5:5117-5123 (1986)).

Chimeric antibodies can be produced by recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding to the antigen. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In some embodiments, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody molecule can be humanized by methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibody molecules can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987. Winter U.S. Pat. No. 5,225,539), the contents of which is expressly incorporated by reference.

Also within the scope of the invention are humanized antibody molecules in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The antibody molecule can be a single chain antibody. A single-chain antibody (scFV) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered. e.g., mutated, to modify the properties of the antibody (e.g., to increase or decrease one or more of: Fc receptor binding, antibody glycosylation, the number of cysteine residues, effector cell function, and/or complement function). In some embodiments the antibody has: effector function; and can fix complement. In other embodiments the antibody does not: recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Similar type of alterations could be described which if applied to the murine, or other species immunoglobulin would reduce or eliminate these functions.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the invention are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

### CDR-grafted Scaffolds

In some embodiments, the antibody molecule is a CDR-grafted scaffold domain. In some embodiments, the scaffold domain is based on a fibronectin domain, e.g., fibronectin type III domain. The overall fold of the fibronectin type III (Fn3) domain is closely related to that of the smallest functional antibody fragment, the variable domain of the antibody heavy chain. There are three loops at the end of Fn3; the positions of BC, DE and FG loops approximately correspond to those of CDR1, 2 and 3 of the VH domain of an antibody. Fn3 does not have disulfide bonds; and therefore Fn3 is stable under reducing conditions, unlike antibodies and their fragments (see, e.g., WO 98/56915; WO 01/64942: WO 00/34784). An Fn3 domain can be modified (e.g., using CDRs or hypervariable loops described herein) or varied, e.g., to select domains that bind to an antigen/marker/cell described herein.

In some embodiments, a scaffold domain, e.g., a folded domain, is based on an antibody, e.g., a “minibody” scaffold created by deleting three beta strands from a heavy chain variable domain of a monoclonal antibody (see, e.g., Tramontano et al., 1994, J Mol. Recognit. 7:9; and Martin et al., 1994, EMBO J. 13:5303-5309). The “minibody” can be used to present two hypervariable loops. In some embodiments, the scaffold domain is a V-like domain (see, e.g., Coia et al. WO 99/45110) or a domain derived from tendamistatin, which is a 74 residue, six-strand beta sheet sandwich held together by two disulfide bonds (see, e.g., McConnell and Hoess, 1995, J Mol. Biol. 250:460). For example, the loops of tendamistatin can be modified (e.g., using CDRs or hypervariable loops) or varied, e.g., to select domains that bind to a marker/antigen/cell described herein. Another exemplary scaffold domain is a beta-sandwich structure derived from the extracellular domain of CTLA-4 (see, e.g., WO 00/60070).

Other exemplary scaffold domains include but are not limited to T-cell receptors; MHC proteins; extracellular domains (e.g., fibronectin Type III repeats, EGF repeats); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases (particularly inactivated proteases), RNase; chaperones, e.g., thioredoxin, and heat shock proteins; and intracellular signaling domains (such as SH2 and SH3 domains). See, e.g., US 20040009530 and U.S. Pat. No. 7,501,121, incorporated herein by reference.

In some embodiments, a scaffold domain is evaluated and chosen. e.g., by one or more of the following criteria: (1) amino acid sequence, (2) sequences of several homologous domains, (3) 3-dimensional structure, and/or (4) stability data over a range of pH, temperature, salinity, organic solvent, oxidant concentration. In some embodiments, the scaffold domain is a small, stable protein domain, e.g., a protein of less than 100, 70, 50, 40 or 30 amino acids. The domain may include one or more disulfide bonds or may chelate a metal, e.g., zinc.

### Antibody-Based Fusions

A variety of formats can be generated which contain additional binding entities attached to the N or C terminus of antibodies. These fusions with single chain or disulfide stabilized Fvs or Fabs result in the generation of tetravalent molecules with bivalent binding specificity for each antigen. Combinations of scFvs and scFabs with IgGs enable the production of molecules which can recognize three or more different antigens.

### Antibody-Fab Fusion

Antibody-Fab fusions are bispecific antibodies comprising a traditional antibody to a first target and a Fab to a second target fused to the C terminus of the antibody heavy chain. Commonly the antibody and the Fab will have a common light chain. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Antibody-scFv Fusion

Antibody-scFv Fusions are bispecific antibodies comprising a traditional antibody and a scFv of unique specificity fused to the C terminus of the antibody heavy chain. The scFv can be fused to the C terminus through the Heavy Chain of the scFv either directly or through a linker peptide. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Variable Domain Immunoglobulin DVD

A related format is the dual variable domain immunoglobulin (DVD), which are composed of VH and VL domains of a second specificity place upon the N termini of the V domains by shorter linker sequences.

Other exemplary multispecific antibody formats include, e.g., those described in the following US20160114057A1, US20130243775A1, US20140051833, US20130022601, US20150017187A1, US20120201746A1, US20150133638A1, US20130266568A1, US20160145340A1, WO2015127158A1, US20150203591A1, US20140322221A1, US20130303396A1, US20110293613, US20130017200A1, US20160102135A1, WO2015197598A2, WO2015197582A1, U.S. Pat. No. 9,359,437, US20150018529, WO2016115274A1, WO2016087416A1, US20080069820A1, U.S. Pat. Nos. 9,145,588B, 7,919,257, and US20150232560A1. Exemplary multispecific molecules utilizing a full antibody-Fab/scFab format include those described in the following, U.S. Pat. No. 9,382,323B2, US20140072581A1, US20140308285A1, US20130165638A1, US20130267686A1, US20140377269A1, U.S. Pat. No. 7,741,446B2, and WO1995009917A1. Exemplary multispecific molecules utilizing a domain exchange format include those described in the following, US20150315296A1, WO2016087650A1, US20160075785A1, WO2016016299A1, US20160130347A1, US20150166670, U.S. Pat. No. 8,703,132B2, US20100316645, U.S. Pat. No. 8,227,577B2, US20130078249.

### Fc-Containing Multispecific Molecules

In some embodiments, the multispecific molecules as described herein includes an immunoglobulin constant region (e.g., an Fc region). Exemplary Fc regions can be chosen from the heavy chain constant regions of IgG1, IgG2, IgG3 or IgG4; more particularly, the heavy chain constant region of human IgG1, IgG2, IgG3, or IgG4.

In some embodiments, the immunoglobulin chain constant region (e.g., the Fc region) is altered. e.g., mutated, to increase or decrease one or more of: Fc receptor binding, antibody glycosylation, the number of cysteine residues, effector cell function, or complement function.

In other embodiments, an interface of a first and second immunoglobulin chain constant regions (e.g., a first and a second Fc region) is altered, e.g., mutated, to increase or decrease dimerization, e.g., relative to a non-engineered interface, e.g., a naturally-occurring interface. For example, dimerization of the immunoglobulin chain constant region (e.g., the Fc region) can be enhanced by providing an Fc interface of a first and a second Fc region with one or more of: a paired protuberance-cavity (“knob-in-a hole”), an electrostatic interaction, or a strand-exchange, such that a greater ratio of heteromultimer to homomultimer forms, e.g., relative to a non-engineered interface.

In some embodiments, the multispecific molecules include a paired amino acid substitution at a position chosen from one or more of 347, 349, 350, 351, 366, 368, 370, 392, 394, 395, 397, 398, 399, 405, 407, or 409, e.g., of the Fc region of human IgG1 For example, the immunoglobulin chain constant region (e.g., Fc region) can include a paired an amino acid substitution chosen from: T366S, L368A, or Y407V (e.g., corresponding to a cavity or hole), and T366W (e.g., corresponding to a protuberance or knob).

In other embodiments, the multifunctional molecule includes a half-life extender, e.g., a human serum albumin or an antibody molecule to human serum albumin.

In some embodiments, Fc contains exemplary Fc modifications listed in Table 14.

### Heterodimerized Antibody Molecules & Methods of Making

Various methods of producing multispecific antibodies have been disclosed to address the problem of incorrect heavy chain pairing. Exemplary methods are described below. Exemplary multispecific antibody formats and methods of making said multispecific antibodies are also disclosed in e.g., Speiss et al. Molecular Immunology 67 (2015) 95-106; and Klein et al mAbs 4:6, 653-663; November/December 2012: the entire contents of each of which are incorporated by reference herein.

Heterodimerized bispecific antibodies are based on the natural IgG structure, wherein the two binding arms recognize different antigens. IgG derived formats that enable defined monovalent (and simultaneous) antigen binding are generated by forced heavy chain heterodimerization, combined with technologies that minimize light chain mispairing (e.g., common light chain). Forced heavy chain heterodimerization can be obtained using, e.g., knob-in-hole OR strand exchange engineered domains (SEED).

### Knob-in-Hole

Knob-in-Hole as described in U.S. Pat. Nos. 5,731,116, 7,476,724 and Ridgway, J. et al. (1996) Prot. Engineering 9(7): 617-621, broadly involves: (1) mutating the CH3 domain of one or both antibodies to promote heterodimerization; and (2) combining the mutated antibodies under conditions that promote heterodimerization. “Knobs” or “protuberances” are typically created by replacing a small amino acid in a parental antibody with a larger amino acid (e.g., T366Y or T366W); “Holes” or “cavities” are created by replacing a larger residue in a parental antibody with a smaller amino acid (e.g., Y407T, T366S, L368A and/or Y407V).

Exemplary “hole” heavy chains are provided in Table 23. In some embodiments, the “hole” heavy chain comprises one or more (e.g., three) heavy chain CDRs provided in Table 23, or a sequence with at least 95% sequence identity thereto.

Exemplary “hole” light chains are provided in Table 23. In some embodiments, the “hole” heavy chain comprises one or more (e.g., three) heavy chain CDRs provided in Table 23, or a sequence with at least 95% sequence identity thereto.

For bispecific antibodies including an Fc domain, introduction of specific mutations into the constant region of the heavy chains to promote the correct heterodimerization of the Fc portion can be utilized. Several such techniques are reviewed in Klein et al. (mAbs (2012) 4:6, 1-11), the contents of which are incorporated herein by reference in their entirety. These techniques include the “knobs-into-holes” (KiH) approach which involves the introduction of a bulky residue into one of the CH3 domains of one of the antibody heavy chains. This bulky residue fits into a complementary “hole” in the other CH3 domain of the paired heavy chain so as to promote correct pairing of heavy chains (see e.g., U.S. Pat. No. 7,642,228).

Exemplary “knob” chains are provided in Table 23. In some embodiments, the “knob” chain comprises a sequence provided in Table 23, or a sequence with at least 95% sequence identity thereto.

Exemplary KiH mutations include S354C, T366W in the “knob” heavy chain and Y349C, T366S, L368A, Y407V in the “hole” heavy chain. Other exemplary KiH mutations are provided in Table 4, with additional optional stabilizing Fc cysteine mutations.

Other Fc mutations are provided by Igawa and Tsunoda who identified 3 negatively charged residues in the CH3 domain of one chain that pair with three positively charged residues in the CH3 domain of the other chain. These specific charged residue pairs are: E356-K439, E357-K370, D399-K409 and vice versa. By introducing at least two of the following three mutations in chain A: E356K, E357K and D399K, as well as K370E, K409D, K439E in chain B, alone or in combination with newly identified disulfide bridges, they were able to favor very efficient heterodimerization while suppressing homodimerization at the same time (Martens T et al. A novel one-armed antic-Met antibody inhibits glioblastoma growth in vivo. Clin Cancer Res 2006; 12:6144-52; PMID:17062691). Xencor defined 41 variant pairs based on combining structural calculations and sequence information that were subsequently screened for maximal heterodimerization, defining the combination of S364H, F405A (HA) on chain A and Y349T, T394F on chain B (TF) (Moore G L et al. A novel bispecific antibody format enables simultaneous bivalent and monovalent co-engagement of distinct target antigens. MAbs 2011; 3:546-57; PMID: 22123055).

Other exemplary Fc mutations to promote heterodimerization of multispecific antibodies include those described in the following references, the contents of each of which is incorporated by reference herein, WO2016071377A1, US20140079689A1, US20160194389A1, US20160257763, WO2016071376A2, WO2015107026A1, WO2015107025A1, WO2015107015A1, US20150353636A1, US20140199294A1, U.S. Pat. No. 7,750,128B2, US20160229915A1, US20150344570A1, U.S. Pat. No. 8,003,774A1, US20150337049A1, US20150175707A1, US20140242075A1, US20130195849A1, US20120149876A1, US20140200331A1, U.S. Pat. No. 9,309,311B2, U.S. Pat. No. 8,586,713, US20140037621A1, US20130178605A1, US20140363426A1, US20140051835A1 and US20110054151A1.

Stabilizing cysteine mutations have also been used in combination with KiH and other Fc heterodimerization promoting variants, see e.g., U.S. Pat. No. 7,183,076. Other exemplary cysteine modifications include, e.g., those disclosed in US20140348839A1, U.S. Pat. No. 7,855,275B2, and U.S. Pat. No. 9,000,130B2.

### Strand Exchange Engineered Domains (SEED)

Heterodimeric Fc platform that support the design of bispecific and asymmetric fusion proteins by devising strand-exchange engineered domain (SEED) C(H)3 heterodimers are known. These derivatives of human IgG and IgA C(H)3 domains create complementary human SEED C(H)3 heterodimers that are composed of alternating segments of human IgA and IgG C(H)3 sequences. The resulting pair of SEED C(H)3 domains preferentially associates to form heterodimers when expressed in mammalian cells. SEEDbody (Sb) fusion proteins consist of [IgG1 hinge]-C(H)2-[SEED C(H)3], that may be genetically linked to one or more fusion partners (see e.g., Davis J H et al. SEEDbodies: fusion proteins based on strand exchange engineered domain (SEED) CH3 heterodimers in an Fc analogue platform for asymmetric binders or immunofusions and bispecific antibodies. Protein Eng Des Sel 2010; 23:195-202; PMID:20299542 and U.S. Pat. No. 8,871,912. The contents of each of which are incorporated by reference herein).

### Fe-Containing Entities (Mini-Antibodies)

Fc-containing entities, also known as mini-antibodies, can be generated by fusing scFv to the C-termini of constant heavy region domain 3 (CH3-scFv) and/or to the hinge region (scFv-hinge-Fc) of an antibody with a different specificity. Trivalent entities can also be made which have disulfide stabilized variable domains (without peptide linker) fused to the C-terminus of CH3 domains of IgGs.

### Duobody

“Duobody” technology to produce bispecific antibodies with correct heavy chain pairing are known. The DuoBody technology involves three basic steps to generate stable bispecific human IgG1 antibodies in a post-production exchange reaction. In a first step, two IgG1s, each containing single matched mutations in the third constant (CH3) domain, are produced separately using standard mammalian recombinant cell lines. Subsequently, these IgG1 antibodies are purified according to standard processes for recovery and purification. After production and purification (post-production), the two antibodies are recombined under tailored laboratory conditions resulting in a bispecific antibody product with a very high yield (typically >95%) (see e.g., Labrijn et al, PNAS 2013; 110(13):5145-5150 and Labrijn et al. Nature Protocols 2014; 9(10):2450-63, the contents of each of which are incorporated by reference herein).

### Electrostatic Interactions

Methods of making multispecific antibodies using CH3 amino acid changes with charged amino acids such that homodimer formation is electrostatically unfavorable are disclosed. EP1870459 and WO 2009089004 describe other strategies for favoring heterodimer formation upon co-expression of different antibody domains in a host cell. In these methods, one or more residues that make up the heavy chain constant domain 3 (CH3), CH3-CH3 interfaces in both CH3 domains are replaced with a charged amino acid such that homodimer formation is electrostatically unfavorable and heterodimerization is electrostatically favorable. Additional methods of making multispecific molecules using electrostatic interactions are described in the following references, the contents of each of which is incorporated by reference herein, include US20100015133, U.S. Pat. No. 8,592,562B2, U.S. Pat. No. 9,200,060B2, US20140154254A1, and U.S. Pat. No. 9,358,286A1.

### Common Light Chain

Light chain mispairing needs to be avoided to generate homogenous preparations of bispecific IgGs. One way to achieve this is through the use of the common light chain principle, i.e. combining two binders that share one light chain but still have separate specificities. An exemplary method of enhancing the formation of a desired bispecific antibody from a mixture of monomers is by providing a common variable light chain to interact with each of the heteromeric variable heavy chain regions of the bispecific antibody. Compositions and methods of producing bispecific antibodies with a common light chain as disclosed in, e.g., U.S. Pat. No. 7,183,076B2, US20110177073A1, EP2847231A1, WO2016079081A1, and EP3055329A1, the contents of each of which is incorporated by reference herein.

### CrossMab

Another option to reduce light chain mispairing is the CrossMab technology which avoids non-specific L chain mispairing by exchanging CH1 and CL domains in the Fab of one half of the bispecific antibody. Such crossover variants retain binding specificity and affinity, but make the two arms so different that L chain mispairing is prevented. The CrossMab technology (as reviewed in Klein et al. Supra) involves domain swapping between heavy and light chains so as to promote the formation of the correct pairings. Briefly, to construct a bispecific IgG-like CrossMab antibody that could bind to two antigens by using two distinct light chain-heavy chain pairs, a two-step modification process is applied. First, a dimerization interface is engineered into the C-terminus of each heavy chain using a heterodimerization approach, e.g., Knob-into-hole (KiH) technology, to ensure that only a heterodimer of two distinct heavy chains from one antibody (e.g., Antibody A) and a second antibody (e.g., Antibody B) is efficiently formed. Next, the constant heavy 1 (CH1) and constant light (CL) domains of one antibody are exchanged (Antibody A), keeping the variable heavy (VH) and variable light (VL) domains consistent. The exchange of the CH1 and CL domains ensured that the modified antibody (Antibody A) light chain would only efficiently dimerize with the modified antibody (antibody A) heavy chain, while the unmodified antibody (Antibody B) light chain would only efficiently dimerize with the unmodified antibody (Antibody B) heavy chain; and thus only the desired bispecific CrossMab would be efficiently formed (see e.g., Cain, C. SciBX 4(28); doi:10.1038/scibx.2011.783, the contents of which are incorporated by reference herein).

### Common Heavy Chain

An exemplary method of enhancing the formation of a desired bispecific antibody from a mixture of monomers is by providing a common variable heavy chain to interact with each of the heteromeric variable light chain regions of the bispecific antibody. Compositions and methods of producing bispecific antibodies with a common heavy chain are disclosed in, e.g., US20120184716, US20130317200, and US20160264685A1, the contents of each of which is incorporated by reference herein.

### Amino Acid Modifications

Alternative compositions and methods of producing multispecific antibodies with correct light chain pairing include various amino acid modifications. For example, Zymeworks describes heterodimers with one or more amino acid modifications in the CH1 and/or CL domains, one or more amino acid modifications in the VH and/or VL domains, or a combination thereof, which are part of the interface between the light chain and heavy chain and create preferential pairing between each heavy chain and a desired light chain such that when the two heavy chains and two light chains of the heterodimer pair are co-expressed in a cell, the heavy chain of the first heterodimer preferentially pairs with one of the light chains rather than the other (see e.g., WO2015181805). Other exemplary methods are described in WO2016026943 (Argen-X), US20150211001, US20140072581A1, US20160039947A1, and US20150368352.

### Lambda/Kappa Formats

Multispecific molecules (e.g., multispecific antibody molecules) that include the lambda light chain polypeptide and a kappa light chain polypeptides, can be used to allow for heterodimerization. Methods for generating bispecific antibody molecules comprising the lambda light chain polypeptide and a kappa light chain polypeptides are disclosed in PCT/US17/53053 filed on Sep. 22, 2017 and designated publication number WO 2018/057955, incorporated herein by reference in its entirety.

In some embodiments, the multispecific molecule includes a multispecific antibody molecule, e.g., an antibody molecule comprising two binding specificities, e.g., a bispecific antibody molecule. The multispecific antibody molecule includes:
a lambda light chain polypeptide 1 (LLCP1) specific for a first epitope;
a heavy chain polypeptide 1 (HCP1) specific for the first epitope;
a kappa light chain polypeptide 2 (KLCP2) specific for a second epitope; and
a heavy chain polypeptide 2 (HCP2) specific for the second epitope.

“Lambda light chain polypeptide 1 (LLCP1)”, as that term is used herein, refers to a polypeptide comprising sufficient light chain (LC) sequence, such that when combined with a cognate heavy chain variable region, can mediate specific binding to its epitope and complex with an HCP1. In some embodiments, it comprises all or a fragment of a CH1 region. In some embodiments, an LLCP1 comprises LC-CDR1, LC-CDR2, LC-CDR3, FR1, FR2, FR3, FR4, and CH1, or sufficient sequence therefrom to mediate specific binding of its epitope and complex with an HCP1. LLCP1, together with its HCP1, provide specificity for a first epitope (while KLCP2, together with its HCP2, provide specificity for a second epitope). As described elsewhere herein, LLCP1 has a higher affinity for HCP1 than for HCP2.

“Kappa light chain polypeptide 2 (KLCP2)”, as that term is used herein, refers to a polypeptide comprising sufficient light chain (LC) sequence, such that when combined with a cognate heavy chain variable region, can mediate specific binding to its epitope and complex with an HCP2. In some embodiments, it comprises all or a fragment of a CH1 region. In some embodiments, a KLCP2 comprises LC-CDR1, LC-CDR2, LC-CDR3, FR1, FR2, FR3, FR4, and CH1, or sufficient sequence therefrom to mediate specific binding of its epitope and complex with an HCP2. KLCP2, together with its HCP2, provide specificity for a second epitope (while LLCP1, together with its HCP1, provide specificity for a first epitope).

“Heavy chain polypeptide 1 (HCP1)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In some embodiments, it comprises all or a fragment of a CH1region. In some embodiments, it comprises all or a fragment of a CH2 and/or CH3 region. In some embodiments, an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an LLCP1, (ii) to complex preferentially, as described herein to LLCP1 as opposed to KLCP2; and (iii) to complex preferentially, as described herein, to an HCP2, as opposed to another molecule of HCP1. HCP1, together with its LLCP1, provide specificity for a first epitope (while KLCP2, together with its HCP2, provide specificity for a second epitope).

“Heavy chain polypeptide 2 (HCP2)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In some embodiments, it comprises all or a fragment of a CH1 region. In some embodiments, it comprises all or a fragment of a CH2 and/or CH3 region. In some embodiments, an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an KLCP2, (ii) to complex preferentially, as described herein to KLCP2 as opposed to LLCP1; and (iii) to complex preferentially, as described herein, to an HCP1, as opposed to another molecule of HCP2. HCP2, together with its KLCP2, provide specificity for a second epitope (while LLCP1, together with its HCP1, provide specificity for a first epitope).

In some embodiments, in the multifunctional polypeptide molecule as described herein: LLCP1 has a higher affinity for HCP1 than for HCP2; and/or KLCP2 has a higher affinity for HCP2 than for HCP1.

In some embodiments, the affinity of LLCP1 for HCP1 is sufficiently greater than its affinity for HCP2, such that under preselected conditions, e.g., in aqueous buffer, e.g., at pH 7, in saline, e.g., at pH 7, or under physiological conditions, at least 75, 80, 90, 95, 98, 99, 99.5, or 99.9% of the multispecific antibody molecule molecules have a LLCP1 complexed, or interfaced with, a HCP1.

In some embodiments, in the multifunctional polypeptide molecule as described herein: the HCP1 has a greater affinity for HCP2, than for a second molecule of HCP1; and/or the HCP2 has a greater affinity for HCP1, than for a second molecule of HCP2.

In some embodiments, the affinity of HCP1 for HCP2 is sufficiently greater than its affinity for a second molecule of HCP1, such that under preselected conditions, e.g., in aqueous buffer, e.g., at pH 7, in saline, e.g., at pH 7, or under physiological conditions, at least 75%, 80, 90, 95, 98, 99 99.5 or 99.9% of the multispecific antibody molecule molecules have a HCP1 complexed, or interfaced with, a HCP2.

In another aspect, described herein is a method for making, or producing, a multispecific antibody molecule. The method includes:
(i) providing a first heavy chain polypeptide (e.g., a heavy chain polypeptide comprising one, two, three or all of a first heavy chain variable region (first VH), a first CH1, a first heavy chain constant region (e.g., a first CH2, a first CH3, or both));
(ii) providing a second heavy chain polypeptide (e.g., a heavy chain polypeptide comprising one, two, three or all of a second heavy chain variable region (second VH), a second CH1, a second heavy chain constant region (e.g., a second CH2, a second CH3, or both));
(iii) providing a lambda chain polypeptide (e.g., a lambda light variable region (VLλ), a lambda light constant chain (VLλ), or both) that preferentially associates with the first heavy chain polypeptide (e.g., the first VH); and
(iv) providing a kappa chain polypeptide (e.g., a lambda light variable region (VLλ), a lambda light constant chain (VLλ), or both) that preferentially associates with the second heavy chain polypeptide (e.g., the second VH), under conditions where (i)-(iv) associate.

In some embodiments, the first and second heavy chain polypeptides form an Fc interface that enhances heterodimerization.

In some embodiments. (i)-(iv) (e.g., nucleic acid encoding (i)-(iv)) are introduced in a single cell, e.g., a single mammalian cell, e.g., a CHO cell. In some embodiments, (i)-(iv) are expressed in the cell. In some embodiments, (i)-(iv) (e.g., nucleic acid encoding (i)-(iv)) are introduced in different cells, e.g., different mammalian cells, e.g., two or more CHO cell. In some embodiments, (i)-(iv) are expressed in the cells.

In some embodiments, the method further comprises purifying a cell-expressed antibody molecule, e.g., using a lambda- and/or -kappa-specific purification, e.g., affinity chromatography.

In some embodiments, the method further comprises evaluating the cell-expressed multispecific antibody molecule. For example, the purified cell-expressed multispecific antibody molecule can be analyzed by techniques known in the art, include mass spectrometry. In some embodiments, the purified cell-expressed antibody molecule is cleaved, e.g., digested with papain to yield the Fab moieties and evaluated using mass spectrometry.

In some embodiments, the method produces correctly paired kappa/lambda multispecific, e.g., bispecific, antibody molecules in a high yield, e.g., at least 75%, 80, 90, 95, 98, 99 99.5 or 99.9%.

In other embodiments, the multispecific, e.g., a bispecific, antibody molecule that includes:
(i) a first heavy chain polypeptide (HCP1) (e.g., a heavy chain polypeptide comprising one, two, three or all of a first heavy chain variable region (first VH), a first CH1, a first heavy chain constant region (e.g., a first CH2, a first CH3, or both)), e.g., wherein the HCP1 binds to a first epitope;
(ii) a second heavy chain polypeptide (HCP2) (e.g., a heavy chain polypeptide comprising one, two, three or all of a second heavy chain variable region (second VH), a second CH1, a second heavy chain constant region (e.g., a second CH2, a second CH3, or both)), e.g., wherein the HCP2 binds to a second epitope;
(iii) a lambda light chain polypeptide (LLCP1) (e.g., a lambda light variable region (VLλ), a lambda light constant chain (VLλ), or both) that preferentially associates with the first heavy chain polypeptide (e.g., the first VH), e.g., wherein the LLCP1 binds to a first epitope; and
(iv) a kappa light chain polypeptide (KLCP2) (e.g., a kappa light variable region (VLκ), a kappa light constant chain (VLκ), or both) that preferentially associates with the second heavy chain polypeptide (e.g., the second VH), e.g., wherein the KLCP2 binds to a second epitope.

In some embodiments, the first and second heavy chain polypeptides form an Fc interface that enhances heterodimerization. In some embodiments, the multispecific antibody molecule has a first binding specificity that includes a hybrid VLλ-CLλheterodimerized to a first heavy chain variable region connected to the Fc constant, CH2-CH3 domain (having a knob modification) and a second binding specificity that includes a hybrid VLκ-CLκ heterodimerized to a second heavy chain variable region connected to the Fe constant, CH2-CH3 domain (having a hole modification).

### Multispecific or Multifunctional Antibody Molecules

Exemplary structures of multispecific and multifunctional molecules defined herein are described throughout. Exemplary structures are further described in: Weidle U et al. (2013) The Intriguing Options of Multispecific Antibody Formats for Treatment of Cancer. Cancer Genomics & Proteomics 10: 1-18 (2013); and Spiess C et al. (2015) Alternative molecular formats and therapeutic applications for bispecific antibodies. Molecular Immunology 67: 95-106: the full contents of each of which is incorporated by reference herein).

In some embodiments, multispecific antibody molecules can comprise more than one antigen-binding site, where different sites are specific for different antigens. In some embodiments, multispecific antibody molecules can bind more than one (e.g., two or more) epitopes on the same antigen. In some embodiments, multispecific antibody molecules comprise an antigen-binding site specific for a target cell (e.g., cancer cell) and a different antigen-binding site specific for an immune effector cell. In some embodiments, the multispecific antibody molecule is a bispecific antibody molecule. Bispecific antibody molecules can be classified into five different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates.

BsIgG is a format that is monovalent for each antigen. Exemplary BsIgG formats include but are not limited to crossMab, DAF (two-in-one), DAF (four-in-one), DutaMab, DT-IgG, knobs-in-holes common LC, knobs-in-holes assembly, charge pair, Fab-arm exchange, SEEDbody, triomab, LUZ-Y, Fcab, κλ-body, orthogonal Fab. See Spiess et al. Mol. Immunol. 67(2015):95-106. Exemplary BsIgGs include catumaxomab (Fresenius Biotech, Trion Pharma, Neopharm), which contains an anti-CD3 arm and an anti-EpCAM arm; and ertumaxomab (Neovii Biotech, Fresenius Biotech), which targets CD3 and HER2. In some embodiments, BsIgG comprises heavy chains that are engineered for heterodimerization. For example, heavy chains can be engineered for heterodimerization using a “knobs-into-holes” strategy, a SEED platform, a common heavy chain (e.g., in KX-bodies), and use of heterodimeric Fc regions. See Spiess et al. Mol. Immunol. 67(2015):95-106. Strategies that have been used to avoid heavy chain pairing of homodimers in BsIgG include knobs-in-holes, duobody, asymmetric, charge pair, HA-TF. SEEDbody, and differential protein A affinity. See Id. BsIgG can be produced by separate expression of the component antibodies in different host cells and subsequent purification/assembly into a BsIgG. BsIgG can also be produced by expression of the component antibodies in a single host cell. BsIgG can be purified using affinity chromatography. e.g., using protein A and sequential pH elution.

IgG appended with an additional antigen-binding moiety is another format of bispecific antibody molecules. For example, monospecific IgG can be engineered to have bispecificity by appending an additional antigen-binding unit onto the monospecific IgG, e.g., at the N- or C-terminus of either the heavy or light chain. Exemplary additional antigen-binding units include single domain antibodies (e.g., variable heavy chain or variable light chain), engineered protein scaffolds, and paired antibody variable domains (e.g., single chain variable fragments or variable fragments). See Id. Examples of appended IgG formats include dual variable domain IgG (DVD-Ig), IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, zybody, and DVI-IgG (four-in-one). See Spiess et al. Mol. Immunol. 67(2015):95-106. An example of an IgG-scFv is MM-141 (Merrimack Pharmaceuticals), which binds IGF-1Rand HER3. Examples of DVD-Ig include ABT-981 (AbbVie), which binds IL-1α and IL-1β; and ABT-122 (AbbVie), which binds TNF and IL-17A.

Bispecific antibody fragments (BsAb) are a format of bispecific antibody molecules that lack some or all of the antibody constant domains. For example, some BsAb lack an Fc region. In some embodiments, bispecific antibody fragments include heavy and light chain regions that are connected by a peptide linker that permits efficient expression of the BsAb in a single host cell. Exemplary bispecific antibody fragments include but are not limited to nanobody, nanobody-HAS, BiTE, Diabody, DART, TandAb, scDiabody, scDiabody-CH3, Diabody-CH3, triple body, miniantibody, minibody, TriBi minibody, scFv-CH3 KIH, Fab-scFv, scFv-CH-CL-scFv, F(ab′)2, F(ab′)2-scFv2, scFv-KIH, Fab-scFv-Fc, tetravalent HCAb, scDiabody-Fc, Diabody-Fc, tandem scFv-Fc, and intrabody. See Id. For example, the BiTE format comprises tandem scFvs, where the component scFvs bind to CD3 on T cells and a surface antigen on cancer cells.

Bispecific fusion proteins include antibody fragments linked to other proteins, e.g., to add additional specificity and/or functionality. An example of a bispecific fusion protein is an immTAC, which comprises an anti-CD3 scFv linked to an affinity-matured T-cell receptor that recognizes HLA-presented peptides. In some embodiments, the dock-and-lock (DNL) method can be used to generate bispecific antibody molecules with higher valency. Also, fusions to albumin binding proteins or human serum albumin can be extend the serum half-life of antibody fragments. See Id.

In some embodiments, chemical conjugation, e.g., chemical conjugation of antibodies and/or antibody fragments, can be used to create BsAb molecules. See Id. An exemplary bispecific antibody conjugate includes the CovX-body format, in which a low molecular weight drug is conjugated site-specifically to a single reactive lysine in each Fab arm or an antibody or fragment thereof. In some embodiments, the conjugation improves the serum half-life of the low molecular weight drug. An exemplary CovX-body is CVX-241 (NCT01004822), which comprises an antibody conjugated to two short peptides inhibiting either VEGF or Ang2. Se Id.

The antibody molecules can be produced by recombinant expression, e.g., of at least one or more component, in a host system. Exemplary host systems include eukaryotic cells (e.g., mammalian cells, e.g., CHO cells, or insect cells, e.g., SF9 or S2 cells) and prokaryotic cells (e.g., E. coli). Bispecific antibody molecules can be produced by separate expression of the components in different host cells and subsequent purification/assembly. Alternatively, the antibody molecules can be produced by expression of the components in a single host cell. Purification of bispecific antibody molecules can be performed by various methods such as affinity chromatography, e.g., using protein A and sequential pH elution. In other embodiments, affinity tags can be used for purification. e.g., histidine-containing tag, myc tag, or streptavidin tag.

### Exemplary Bispecific Molecules

In an aspect, a multispecific molecule as described herein comprises a sequence as described herein, e.g., a sequence chosen from SEQ ID NOs: 1004-1007, 3275-3277, 3286, or 3287, or a sequence with at least 85%, 90%, 95%, 96%, 97%, 98%, 99% or more identity thereto. In some embodiments, a multispecific molecule as described herein comprises a leader sequence comprising the amino acid sequence of SEQ ID NO: 3288. In some embodiments, a multispecific molecule as described herein does not comprise a leader sequence comprising the amino acid sequence of SEQ ID NO: 3288.

Molecule F: aCD19×aVb6.5: Molecule F comprises a heavy chain comprising the amino acid sequence of SEQ ID NO: 1004 and alight chain comprising the amino acid sequence of SEQ ID NO: 1005.

Molecule F.1
(heavy chain) (Tcrvbeta6_5 scFv/anti-CD19 heavy chain)
SEQ ID NO: 1004
METDTLLLWVLLLWVPGSTGQVQLVQSGAEVKKPGSSVKVSCKASGYSFTTYYIHWVRQAPGQ
GLEWMGWFFPGSGNIKYNEKFKGRVTITADTSTSTAYMELSSLRSEDTAVYYCAGSYYSYDVLD
YWGQGTTVTVSSGGGGSGGGGSGGGGSGGGGSDIQMTQSPSFLSASVGDRVTITCKASQNVGIN
VVWHQQKPGKAPKALIYSSSHRYSGVPSRFSGSGSGTEFTLTISSLQPEDFATYFCQQFKSYPLTF
GQGTKLEIKGGGGSQVTLRESGPALVKPTQTLTLTCTFSGFSLSTSGMGVGWIRQPPGKALEWLA
HIWWDDDKRYNPALKSRLTISKDTSKNQVFLTMTNMDPVDTATYYCARMELWSYYFDYWGQ
GTTVTVSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYASTYRVVS
VLTVLHQDWLNGKEYKCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGNVFSCSVMHEAL
HNHYTQKSLSLSPGK
Molecule F.2
(light chain) (anti-CD19 light chain)
SEQ ID NO: 1005
METPAQLLFLLLLWLPDTTGENVLTQSPATLSLSPGERATLSCSASSSVSYMHWYQQKPGQAPR
LLIYDTSKLASGIPARFSGSGSGTDHTLTISSLEPEDFAVYYCFQGSVYPFTFGQGTKLEIKRTVAA
PSVFIFPPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC

In an aspect, a multispecific molecule as described herein comprises SEQ ID NO: 1004 and/or SEQ ID NO: 1005 or a sequence with at least 85%, 90%, 95%, 96%, 97%, 98%, 99% or more identity thereto.

Molecule G: aBCMA×aVb6.5: Molecule G comprises a heavy chain comprising the amino acid sequence of SEQ ID NO: 1006 and alight chain comprising the amino acid sequence of SEQ ID NO: 1007.

Molecule G.1
(heavy chain)
SEQ ID NO: 1006
METDTLLLWVLLLWVPGSTGQVQLVQSGAEVKKPGSSVKVSCKASGYSFTTYYIHWVRQAPGQ
GLEWMGWFFPGSGNIKYNEKFKGRVTITADTSTSTAYMELSSLRSEDTAVYYCAGSYYSYDVLD
YWGQGTTVTVSSGGGGSGGGGSGGGGSGGGGSDIQMTQSPSFLSASVGDRVTITCKASQNVGIN
VVWHQQKPGKAPKALIYSSSHRYSGVPSRFSGSGSGTEFTLTISSLQPEDFATYFCQQFKSYPLTF
GQGTKLEIKGGGGSQVQLVESGGGVVQPGRSLRLSCAASGIDFSRYWMSWVRQAPGKGLEWV
GEINPDSSTINYAPSLKDRFTISRDNSKNTLYLQMSSLRAEDTAVYYCASLYYDYGDAMDYWGQ
GTTVTVSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYASTYRVVS
VLTVLHQDWLNGKEYKCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGNVFSCSVMHEAL
HNRFTQKSLSLSPGK
Molecule G.2
(light chain)
SEQ ID NO: 1007
METDTLLLWVLLLWVPGSTGDIQMTQSPSSLSASVGDRVTITCKASQSVDSNVAWYQQKPEKAP
KALIFSASLRFSGVPSRFSGSGSGTDFTLTISSLQPEDFATYFCQQYNNYPLTFGQGTKLEIKRTVA
APSVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC

In an aspect, a multispecific molecule as described herein comprises SEQ ID NO: 1006 and/or SEQ ID NO: 1007 or a sequence with at least 85%, 90%, 95%, 96%, 97%, 98%, 99% or more identity thereto.

Molecule H: aBCMA×aTCRvbeta6_5: Molecule H comprises a first heavy chain comprising the amino acid sequence of SEQ ID NO: 3275, a light chain comprising the amino acid sequence of SEQ ID NO: 3277, and a second heavy chain comprising the amino acid sequence of SEQ ID NO: 3276.

Molecule H.1
(anti-BCMA heavy chain)
SEQ ID NO: 3275
METDTLLLWVLLLWVPGSTGQVQLVESGGGVVQPGRSLRLSCAASGIDFSRYWMSWVRQAPG
KGLEWVGEINPDSSTINYAPSLKDRFTISRDNSKNTLYLQMSSLRAEDTAVYYCASLYYDYGDA
MDYWGQGTTVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEPKSCDKTHTCPPCPAPE
LLGGPSVFLFPPKPKDTLMISRTPEVTCVVVDVSHEDPEVKFNWYVDGVEVHNAKTKPREEQYN
ATYRVVSVLTVLHQDWLNGKEYKCKVSNKALPAPIEKTISKAKGQPREPQVYTLPPCREEMTKN
QVSLWCLVKGFYPSDIAVEWESNGQPENNYKTTPPVLDSDGSFFLYSKLTVDKSRWQQGNVFSC
SVMHEALHNHYTQKSLSLSPGK
Molecule H.2
(TCRvbeta_6_5 scFv humanized)
SEQ ID NO: 3276
METDTLLLWVLLLWVPGSTGQVQLVQSGAEVKKPGSSVKVSCKASGYSFTTYYIHWVRQAPGQ
GLEWMGWFFPGSGNIKYNEKFKGRVTITADTSTSTAYMELSSLRSEDTAVYYCAGSYYSYDVLD
YWGQGTTVTVSSGGGGSGGGGSGGGGSGGGGSDIQMTQSPSFLSASVGDRVTITCKASQNVGIN
VVWHQQKPGKAPKALIYSSSHRYSGVPSRFSGSGSGTEFTLTISSLQPEDFATYFCQQFKSYPLTF
GQGTKLEIKGGGGSGGGGSDKTHTCPPCPAPELLGGPSVFLFPPKPKDTLMISRTPEVTCVVVDV
SHEDPEVKFNWYVDGVEVHNAKTKPREEQYASTYRVVSVLTVLHQDWLNGKEYKCKVSNKAL
PAPIEKTISKAKGQPREPQVCTLPPSREEMTKNQVSLSCAVKGFYPSDIAVEWESNGQPENNYKT
TPPVLDSDGSFFLVSKLTVDKSRWQQGNVFSCSVMHEALHNHYTQKSLSLSPGK
Molecule H.3
(anti-BCMA light chain)
SEQ ID NO: 3277
METDTLLLWVLLLWVPGSTGDIQMTQSPSSLSASVGDRVTITCKASQSVDSNVAWYQQKPEKAP
KALIFSASLRFSGVPSRFSGSGSGTDFTLTISSLQPEDFATYFCQQYNNYPLTFGQGTKLEIKRTVA
APSVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC

In an aspect, a multispecific molecule as described herein comprises SEQ ID NO: 3275, SEQ ID NO: 3276, and/or SEQ ID NO: 3277 or a sequence with at least 85%, 90%, 95%, 96%, 97%, 98%, 99% or more identity thereto.

Molecule 1: half arm BCMA Fab with c-terminal scFv TCRvbeta: Molecule I comprises a first heavy chain comprising the amino acid sequence of SEQ ID NO: 3286, a light chain comprising the amino acid sequence of SEQ ID NO: 3277, and a second heavy chain comprising the amino acid sequence of SEQ ID NO: 3287.

Molecule I.1
(heavy chain 1)
SEQ ID NO: 3286
METDTLLLWVLLLWVPGSTGQVQLVESGGGVVQPGRSLRLSCAASGIDFSRYWMSWVRQAPG
KGLEWVGEINPDSSTINYAPSLKDRFTISRDNSKNTLYLQMSSLRAEDTAVYYCASLYYDYGDA
MDYWGQGTTVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEPKSCDKTHTCPPCPAPE
LLGGPSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPCREEMTKN
QVSLWCLVKGFYPSDIAVEWESNGQPENNYKTTPPVLDSDGSFFLYSKLTVDKSRWQQGNVFSC
SVMHEALHNHYTQKSLSLSPGKGGGGSGGGGSGGGGSEVQLVESGGGLVQPGGSLRLSCAASG
FTFSTYAMNWVRQAPGKGLEWVSRIRSKYNNYATYYADSVKDRFTISRDDSKNTLYLQMNSLK
TEDTAVYYCVRHGNFGNSYVSWFAYWGQGTLVTVSSGGGGSGGGGSGGGGSGGGGSQAVVT
QEPSLTVSPGGTVTLTCRSSTGAVTTSNYANWVQQKPGQAPRGLIGGTNKRAPWTPARFSGSLL
GGKAALTLSGAQPEDEAEYYCALWYSNLWVFGGGTKLTVL
Molecule I.2
(light chain)
SEQ ID NO: 3277
METDTLLLWVLLLWVPGSTGDIQMTQSPSSLSASVGDRVTITCKASQSVDSNVAWYQQKPEKAP
KALIFSASLRFSGVPSRFSGSGSGTDFTLTISSLQPEDFATYFCQQYNNYPLTFGQGTKLEIKRTVA
APSVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Molecule I.3
(heavy chain 2)
SEQ ID NO: 3287
METDTLLLWVLLLWVPGSTGDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNK
ALPAPIEKTISKAKGQPREPQVCTLPPSREEMTKNQVSLSCAVKGFYPSDIAVEWESNGQPENNY
KTTPPVLDSDGSFFLVSKLTVDKSRWQQGNVFSCSVMHEALHNHYTQKSLSLSPGK

In an aspect, a multispecific molecule as described herein comprises SEQ ID NO: 3286, SEQ ID NO: 3277, and/or SEQ ID NO: 3287 or a sequence with at least 85%, 90%, 95%, 96%, 97%, 98%, 99% or more identity thereto.

In some embodiments, the multispecific or multifunctional molecule as described herein binds to immune cells. In some embodiments, the multispecific or multifunctional molecule as described herein binds to subsets of immune cells. In some embodiments, the multispecific or multifunctional molecule as described herein binds to T cells. In some embodiments, the multispecific or multifunctional molecule as described herein binds to gamma/delta T cells. In some embodiments, the multispecific or multifunctional molecule as described herein binds to NKT cells. In some embodiments, the multispecific or multifunctional molecule as described herein binds to T cells via a binding moiety. Exemplary binding moieties include, but are not limited to, the binding moieties that bind to TRBC1, TRBC2, CD3, TRAC, TRAV subtypes, CD4, CD8, CD2, CD28, 41BB, PD1, CTLA4, OX40, TIM3, or LAG3. In some embodiments, the multispecific or multifunctional molecule as described herein binds to T cells via a binding moiety that binds to TRBC1. In some embodiments, the multispecific or multifunctional molecule as described herein binds to T cells via a binding moiety that binds to TRBC2. In some embodiments, the multispecific or multifunctional molecule as described herein comprises a binding moiety that binds to TRBC1. In some embodiments, the multispecific or multifunctional molecule as described herein comprises a binding moiety that binds to TRBC2.

In some embodiments, in exemplary embodiments of multifunctional molecules comprises a tumor-associated antigen (TAA)-binding moiety, a TCRβV-binding moiety, and at least one cytokine polypeptide as described herein, a tumor-associated antigen (TAA)-binding moiety and/or a TCRβV-binding moiety are antibody, an antigen binding fragment thereof or an antibody fragment. In some embodiments, in exemplary embodiments of multifunctional molecules comprises a tumor-associated antigen (TAA)-binding moiety, a TCRβV-binding moiety, and at least one cytokine polypeptide as described herein, an antigen binding fragment or an antibody fragment comprises Fab, Fab′, F(ab′)₂, F(ab)₂, variable fragment (Fv), domain antibody (dAb), or single chain variable fragment (scFv). In some embodiments, in exemplary embodiments of multifunctional molecules comprises a tumor-associated antigen (TAA)-binding moiety, a TCRβV-binding moiety, and at least one cytokine polypeptide as described herein, the at least one cytokine polypeptide is selected from the group consisting of interleukin-2 (IL-2) or a fragment or a functional fragment or a functional variant thereof, interleukin-7 (IL-7) or a fragment or a functional fragment or a functional variant thereof, interleukin-12 (IL-12) or a fragment or a functional fragment or a functional variant thereof, interleukin-15 (IL-15) or a fragment or a functional fragment or a functional variant thereof, interleukin-18 (IL-18) or a fragment or a functional fragment or a functional variant thereof, interleukin-21 (IL-21) or a fragment or a functional fragment or a functional variant thereof, or interferon gamma or a fragment or a functional fragment or a functional variant thereof, or a combination thereof. In some embodiments, exemplary embodiments of multifunctional molecules comprises a tumor-associated antigen (TAA)-binding moiety, a TCRβV-binding moiety, and at least one cytokine polypeptide as described herein comprises an exemplary dimerization module comprising an Fc region comprising N297A mutation. In some embodiments, exemplary embodiments of multifunctional molecules comprising a tumor-associated antigen (TAA)-binding moiety, a TCRβV-binding moiety, and at least one cytokine polypeptide as described herein comprise an exemplary dimerization module, e.g., an Fc region comprising Knob-in-hole mutations and disulfide bridges. In some embodiments, exemplary embodiments of multifunctional molecules comprising a tumor-associated antigen (TAA)-binding moiety, a TCRβV-binding moiety, and at least one cytokine polypeptide as described herein comprise an exemplary dimerization module, e.g., an Fc region comprising disulfide bridges, but not comprising Knob-in-hole mutations. In some embodiments, exemplary embodiments of multifunctional molecules comprising a tumor-associated antigen (TAA)-binding moiety, a TCRβV-binding moiety, and at least one cytokine polypeptide as described herein comprise an exemplary dimerization module, e.g., an Fc region not comprising disulfide bridges nor Knob-in-hole mutations. In some embodiments, in exemplary embodiment of multifunctional molecules comprising a tumor-associated antigen (TAA)-binding moiety, a TCRβV-binding moiety, and at least one cytokine polypeptide as described herein, the TCRβV-binding moiety comprises an scFv. In some embodiments, in exemplary embodiment of multifunctional molecules comprising a tumor-associated antigen (TAA)-binding moiety, a TCRβV-binding moiety, and at least one cytokine polypeptide as described herein, the TCRβV-binding moiety comprises a Fab.

In some embodiments, exemplary embodiments of multifunctional molecules can comprise a tumor-associated antigen (TAA)-binding moiety, a TCRαV-binding moiety, and at least one cytokine polypeptide as described herein comprises an exemplary dimerization module comprising an Fc region comprising N297A mutation. In some embodiments, exemplary embodiments of multifunctional molecules comprising a tumor-associated antigen (TAA)-binding moiety, a TCRαV-binding moiety, and at least one cytokine polypeptide as described herein comprise an exemplary dimerization module, e.g., an Fc region comprising Knob-in-hole mutations and disulfide bridges. In some embodiments, exemplary embodiments of multifunctional molecules comprising a tumor-associated antigen (TAA)-binding moiety, a TCRαV-binding moiety, and at least one cytokine polypeptide as described herein comprise an exemplary dimerization module, e.g., an Fc region comprising disulfide bridges, but not comprising Knob-in-hole mutations. In some embodiments, exemplary embodiments of multifunctional molecules comprising a tumor-associated antigen (TAA)-binding moiety, a TCRαV-binding moiety, and at least one cytokine polypeptide as described herein comprise an exemplary dimerization module, e.g., an Fc region not comprising disulfide bridges nor Knob-in-hole mutations. In some embodiments, in exemplary embodiment of multifunctional molecules comprising a tumor-associated antigen (TAA)-binding moiety, a TCRαV-binding moiety, and at least one cytokine polypeptide as described herein, the TCRαV-binding moiety comprises an scFv. In some embodiments, in exemplary embodiment of multifunctional molecules comprising a tumor-associated antigen (TAA)-binding moiety, a TCRαV-binding moiety, and at least one cytokine polypeptide as described herein, the TCRαV-binding moiety comprises a Fab.

In some embodiments, the multifunctional molecule as described herein comprises the at least one cytokine molecule or functional fragment or functional variant thereof comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 2270.

In some embodiments, the multifunctional molecule as described herein comprises the at least one cytokine molecule or functional fragment or functional variant thereof comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2270.

In some embodiments, the multifunctional molecule as described herein comprises the first Fc region, the second Fc region, or a combination thereof comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 40, SEQ ID NO: 42, SEQ ID NO: 3645, SEQ ID NO: 3646, SEQ ID NO: 3647, SEQ ID NO:3648, SEQ ID NO: 3649, SEQ ID NO: 3792, or SEQ ID NO: 3794.

In some embodiments, the multifunctional molecule as described herein comprises the first Fc region, the second Fc region, or a combination thereof comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 40, SEQ ID NO: 42, SEQ ID NO: 3645, SEQ ID NO: 3646, SEQ ID NO: 3647, SEQ ID NO:3648, SEQ ID NO: 3649, SEQ ID NO: 3792, or SEQ ID NO: 3794.

In some embodiments, the multifunctional molecule as described herein comprises the first Fc region comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3649 or SEQ ID NO: 3792, the second Fc region comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3648 or SEQ ID NO: 3794, or a combination thereof. In some embodiments, the multifunctional molecule as described herein comprises the first Fc region comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3648 or SEQ ID NO: 3794, the second Fc region comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3648 or SEQ ID NO: 3794, or a combination thereof.

In some embodiments, the multifunctional molecule as described herein comprises the first Fc region comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3649 or SEQ ID NO: 3792, and the second Fc region comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3648 or SEQ ID NO: 3794. In some embodiments, the multifunctional molecule as described herein comprises the first Fc region comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3648 or SEQ ID NO: 3794, and the second Fc region comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3648 or SEQ ID NO: 3794.

In some embodiments, the multifunctional molecule as described herein comprises the first Fc region comprising a sequence having the sequence of SEQ ID NO: 3649 or SEQ ID NO: 3792, the second Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 or SEQ ID NO: 3794, or a combination thereof. In some embodiments, the multifunctional molecule as described herein comprises the first Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 or SEQ ID NO: 3794, the second Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 or SEQ ID NO: 3794, or a combination thereof.

In some embodiments, the multifunctional molecule as described herein comprises the first Fc region comprising a sequence having the sequence of SEQ ID NO: 3649 or SEQ ID NO: 3792, and the second Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 or SEQ ID NO: 3794. In some embodiments, the multifunctional molecule as described herein comprises the first Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 or SEQ ID NO: 3794, and the second Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 or SEQ ID NO: 3794.

In some embodiments, the multifunctional molecule as described herein comprises the TCRβV-binding moiety comprising: (i) a VH comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to any one of the heavy chain variable domain amino acid sequences listed in Tables 1, 2, 10B, 11, 12, 13, or 21; (ii) a VL comprising a sequence having at least at least 50%, 55%, 60%, 65%, 70%, 75%, 80%, 85%, 90%, 95%, 96%, 97%, 98%, 99%, 99.5%, 99.7%, 99.9% sequence identity to any one of the light chain variable domain amino acid sequences listed in Tables 1, 2, 10B, 11, 12, 13, or 21; (iii) a combination thereof.

In some embodiments, the multifunctional molecule as described herein comprises the TCRβV-binding moiety comprising: (i) a VH comprising any one of the heavy chain variable domain amino acid sequences listed in Tables 1, 2, 10B, 11, 12, 13, or 21; (ii) a VL comprising any one of the light chain variable domain amino acid sequences listed in Tables 1, 2, 10B, 11, 12, 13, or 21; (iii) a combination thereof.

In some embodiments, the multifunctional molecule as described herein comprises the second polypeptide comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 1346, a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 1349, a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 2270, and a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3648.

In some embodiments, the multifunctional molecule as described herein comprises the second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346, the sequence of SEQ ID NO: 1349, the sequence of SEQ ID NO: 2270, and the sequence of SEQ ID NO: 3648.

In some embodiments, the multifunctional molecule as described herein comprises the second polypeptide further comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3801, the sequence of SEQ ID NO: 3309, the sequence of SEQ ID NO: 3308, or any combination thereof.

In some embodiments, the multifunctional molecule as described herein comprises the second polypeptide comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 1346 operatively linked to a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 1349 operatively linked to a sequence having at least 50%, 55%, 60%, 65%, 70%, 75%, 80%, 85%, 90%, 95%, %%, 97%, 98%, 99%, 99.5%, 99.7%, or 99.9% sequence identity to the sequence of SEQ ID NO: 2270 operatively linked to a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3648.

In some embodiments, the multifunctional molecule as described herein comprises the sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 1346 operatively linked to the sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 1349 via the sequence of SEQ ID NO: 3801.

In some embodiments, the multifunctional molecule as described herein comprises the sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 1349 operatively linked to the sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 2270 via the sequence of SEQ ID NO: 3309.

In some embodiments, the multifunctional molecule as described herein comprises the sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 2270 operatively linked to the sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3648 via the sequence of SEQ ID NO: 3308.

In some embodiments, the multifunctional molecule as described herein comprises the second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346 operatively linked to the sequence of SEQ ID NO: 1349 operatively linked to the sequence of SEQ ID NO: 2270 operatively linked to the sequence of SEQ ID NO: 3648.

In some embodiments, the multifunctional molecule as described herein comprises the sequence of SEQ ID NO: 1346 operatively linked to the sequence of SEQ ID NO: 1349 via the sequence of SEQ ID NO: 3801.

In some embodiments, the multifunctional molecule as described herein comprises the sequence of SEQ ID NO: 1349 operatively linked to the sequence of SEQ ID NO: 2270 via the sequence of SEQ ID NO: 3309.

In some embodiments, the multifunctional molecule as described herein comprises the sequence of SEQ ID NO: 2270 operatively linked to the sequence of SEQ ID NO: 3648 via the sequence of SEQ ID NO: 3308.

In some embodiments, the multifunctional molecule as described herein comprises the second polypeptide comprising a sequence having at least 50%, 55%, 60%, 65%, 70%, 75%, 80%, 85%, 90%, 95%, 96%, 97%, 98%, 99%, 99.5%, 99.7%, or 99.9% sequence identity to the sequence of SEQ ID NO: 3800.

In some embodiments, the multifunctional molecule as described herein comprises the second polypeptide comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3800.

In some embodiments, the multifunctional molecule as described herein comprises a sequence having at least 50%, 55%, 60%, 65%, 70%, 75%, 80%, 85%, 90%, 95%, 96%, 97%, 98%, 99%, 99.5%, 99.7%, or 99.9% sequence identity to any component sequences listed in Table 24, e.g., the VH sequences of TCRvβ listed in Table 24, the VL sequences of TCRvβ listed in Table 24, Fc region sequences listed in Table 24, cytokine sequences listed in Table 24, immunoglobulin light chain constant region sequences listed in Table 24, and linker sequences listed in Table 24, or any combination thereof. In some embodiments, the multifunctional molecule as described herein comprises a sequence having at least 50%, 55%, 60%, 65%, 70%, 75%, 80%, 85%, 90%, 95%, 96%, 97%, 98%, 99%, 99.5%, 99.7%, or 99.9% sequence identity to any chain or polypeptide chain sequences listed in Table 24.

In some embodiments, the multifunctional molecule as described herein comprises any component sequences listed in Table 24, e.g., the VH sequences of TCRvβ listed in Table 24, the VL sequences of TCRvβ listed in Table 24, Fc region sequences listed in Table 24, cytokine sequences listed in Table 24, immunoglobulin light chain constant region sequences listed in Table 24, and linker sequences listed in Table 24, or any combination thereof. In some embodiments, the multifunctional molecule as described herein comprises any chain or polypeptide chain sequences listed in Table 24.

In some embodiments, the multifunctional molecule as provided herein comprises, from the N-terminus to the C-terminus, the following configuration:

A-,B-[dimerization module]-C,-D.

In some embodiments, the dimerization module comprises a first immunoglobulin chain constant region and a second immunoglobulin chain constant region.

In some embodiments, A and C are linked to the first immunoglobulin chain constant region; and B and D are linked to the second immunoglobulin chain constant region.

In some embodiments, A or C is the tumor-associated antigen binding moiety.

In some embodiments, B and D are independently absent or at least one cytokine molecule or functional fragment or functional variant thereof.

In some embodiments, when A is the tumor-associated antigen binding moiety, C is absent or at least one cytokine molecule or functional fragment or functional variant thereof.

In some embodiments, when C is the tumor-associated antigen binding moiety, A is absent or at least one cytokine molecule or functional fragment or functional variant thereof.

In some embodiments, the Fc region comprises one or more mutations that modify the binding to the Fc gamma receptors. In some embodiments, the Fc region comprises one or more mutations that reduce the binding to Fc gamma receptors. In some embodiments, the Fc region comprises N297A mutation.

In some embodiments the multifunctional molecule consists of two polypeptide chains. In some embodiments the multifunctional molecule consists of two polypeptide chains, wherein the first polypeptide chain comprises an anti-tumor antigen binder, e.g., an scFv that binds to a tumor antigen. In some embodiments the multifunctional molecule consists of two polypeptide chains, wherein the first polypeptide chain comprises an anti-tumor antigen binder, e.g., an scFv that binds to a tumor antigen, linked to an Fc, e.g., a human IgG1 hole N297A. In some embodiments the multifunctional molecule consists of two polypeptide chains, wherein the first polypeptide chain comprises an anti-tumor antigen binder, e.g., an scFv that binds to a tumor antigen, linked to an Fc, e.g., a human IgG1 hole N297A; and the second polypeptide chain that comprises an anti-TCRvB binder linked to a cytokine, e.g., IL-2, wherein the cytokine is linked to an Fc, e.g., a human IgG1 knob cys N297A. In some embodiments the multifunctional molecule consists of two polypeptide chains, wherein the first polypeptide chain comprises an anti-tumor antigen binder, e.g., an scFv that binds to a tumor antigen, linked to an Fc, e.g., a human IgG1 hole N297A; and the second polypeptide chain comprises, from N to C terminus an anti-TCRvB binder linked to a cytokine, e.g., IL-2, linked to an Fc, e.g., a human IgG1 knob cys N297A. In some embodiments the multifunctional molecule consists of two polypeptide chains, wherein the first polypeptide chain comprises an anti-tumor antigen binder, e.g., an scFv that binds to a tumor antigen, linked to an Fc, e.g., a human IgG1 hole N297A; and the second polypeptide chain comprises SEQ ID NO 3800.

In some embodiments the multifunctional molecule consists of three polypeptide chains. In some embodiments the multifunctional molecule consists of three polypeptide chains, wherein the first polypeptide chain comprises a first portion of an anti-tumor antigen binder that binds to a tumor antigen, e.g., a first portion of an Fab, e.g. heavy chain variable domain of a Fab. In some embodiments the multifunctional molecule consists of three polypeptide chains, wherein the first polypeptide chain comprises a first portion of an anti-tumor antigen binder that binds to a tumor antigen, e.g., a first portion of an Fab, e.g. heavy chain variable domain of a Fab, linked to an Fc, e.g., a human IgG1 hole N297A. In some embodiments the multifunctional molecule consists of three polypeptide chains, wherein the first polypeptide chain comprises a first portion of an anti-tumor antigen binder that binds to a tumor antigen, e.g., an first portion of an Fab, e.g. heavy chain variable domain of a Fab; and the second polypeptide chain that comprises an anti-TCRvB binder linked to a cytokine, e.g., IL-2, wherein the cytokine is linked to an Fc, e.g., a human IgG1 knob cys N297A. In some embodiments, the multifunctional molecule consists of three polypeptide chains, wherein the first polypeptide chain comprises a first portion of an anti-tumor antigen binder that binds to a tumor antigen, e.g., an first portion of an Fab, e.g. heavy chain variable domain of a Fab, linked to an Fc, e.g., a human IgG1 hole N297A; and the second polypeptide chain comprises, from N to C terminus an anti-TCRvB binder linked to a cytokine, e.g., IL-2, linked to an Fc, e.g., a human IgG1 knob cys N297A. In some embodiments, the multifunctional molecule consists of three polypeptide chains, wherein the first polypeptide chain comprises a first portion of an anti-tumor antigen binder that binds to a tumor antigen, e.g., an first portion of an Fab, e.g. heavy chain variable domain of a Fab, linked to an Fc, e.g., a human IgG1 hole N297A; and the second polypeptide chain comprises SEQ ID NO 3800. In some embodiments, the multifunctional molecule consists of three polypeptide chains, wherein the first polypeptide chain comprises a first portion of an anti-tumor antigen binder that binds to a tumor antigen, e.g., an first portion of an Fab, e.g. heavy chain variable domain of a Fab, linked to an Fc, e.g., a human IgG1 hole N297A; and the second polypeptide chain comprises, from N to C terminus an anti-TCRvB binder linked to a cytokine, e.g., IL-2, linked to an Fc, e.g., a human IgG1 knob cys N297A; and the third polypeptide chain comprises a second portion of the anti-tumor antigen binder that binds to a tumor antigen, e.g., a second portion of the Fab, e.g. a light chain variable domain of the Fab. In some embodiments, the multifunctional molecule consists of three polypeptide chains, wherein the first polypeptide chain comprises a first portion of an anti-tumor antigen binder that binds to a tumor antigen, e.g., an first portion of an Fab, e.g. heavy chain variable domain of a Fab, linked to an Fc, e.g., a human IgG1 hole N297A; and the second polypeptide chain comprises SEQ ID NO 3800; and the third polypeptide chain comprises a second portion of the anti-tumor antigen binder that binds to a tumor antigen, e.g., a second portion of the Fab, e.g. a light chain variable domain of the Fab.

### Linkers

The multispecific or multifunctional molecule as described herein can further include a linker, e.g., a linker between one or more of: the antigen binding domain and the cytokine molecule, the antigen binding domain and the immune cell engager, the antigen binding domain and the stromal modifying moiety, the cytokine molecule and the immune cell engager, the cytokine molecule and the stromal modifying moiety, the immune cell engager and the stromal modifying moiety, the antigen binding domain and the immunoglobulin chain constant region, the cytokine molecule and the immunoglobulin chain constant region, the immune cell engager and the immunoglobulin chain constant region, or the stromal modifying moiety and the immunoglobulin chain constant region. In some embodiments, the linker is chosen from: a cleavable linker, a non-cleavable linker, a peptide linker, a flexible linker, a rigid linker, a helical linker, or a non-helical linker, or a combination thereof.

In some embodiments, the multispecific molecule can include one, two, three or four linkers, e.g., a peptide linker. In some embodiments, the peptide linker includes Gly and Ser. In some embodiments, the peptide linker is selected from GGGGS (SEQ ID NO: 3307); GGGGSGGGGS (SEQ ID NO: 3308); GGGGSGGGGSGGGGS (SEQ ID NO: 3309); DVPSGPGGGGGSGGGGS (SEQ ID NO: 3310); GGSGGGGSGGGSGGGGSGGG (SEQ ID NO: 3473); and GGGGSGGGGSGGGGGS (SEQ ID NO: 3643). In some embodiments, the peptide linker is a A(EAAAK)nA (SEQ ID NO: 3477) family of linkers (e.g., as described in Protein Eng. (2001) 14 (8): 529-532). These are stiff helical linkers with n ranging from 2-5. In some embodiments, the peptide linker is selected from AEAAAKEAAAKAAA (SEQ ID NO: 3314); AEAAAKEAAAKEAAAKAAA (SEQ ID NO: 3315); AEAAAKEAAAKEAAAKEAAAKAAA (SEQ ID NO: 3316); and AEAAAKEAAAKEAAAKEAAAKEAAAKAAA(SEQ ID NO: 3317).

### Nucleic Acids

Described herein, in certain embodiments, is an isolated nucleic acid molecule comprising a nucleotide sequence having at least 75%, 80%, 85%, 90%, 95%, 99%, 99.5%, 99.9%, or 100% sequence identity to the nucleotide sequence encoding the multifunctional polypeptide molecule as described herein.

Nucleic acids encoding the aforementioned antibody molecules, e.g., anti-TCRβV antibody molecules, anti-TCRαV antibody molecules multispecific or multifunctional molecules are also disclosed.

In certain embodiments, the invention features nucleic acids comprising nucleotide sequences that encode heavy and light chain variable regions and CDRs or hypervariable loops of the antibody molecules, as described herein. For example, the invention features a first and second nucleic acid encoding heavy and light chain variable regions, respectively, of an antibody molecule chosen from one or more of the antibody molecules as described herein. The nucleic acid can comprise a nucleotide sequence as set forth in the tables herein, or a sequence substantially identical thereto (e.g., a sequence at least about 85%, 90%, 95%, 99% or more identical thereto, or which differs by no more than 3, 6, 15, 30, or 45 nucleotides from the sequences shown in the tables herein.

In certain embodiments, the nucleic acid can comprise a nucleotide sequence encoding at least one, two, or three CDRs or hypervariable loops from a heavy chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In other embodiments, the nucleic acid can comprise a nucleotide sequence encoding at least one, two, or three CDRs or hypervariable loops from a light chain variable region having an amino acid sequence as set forth in the tables herein, or a sequence substantially homologous thereto (e.g., a sequence at least about 85%, 90%, 95%, 99% or more identical thereto, and/or having one or more substitutions. e.g., conserved substitutions). In yet another embodiment, the nucleic acid can comprise a nucleotide sequence encoding at least one, two, three, four, five, or six CDRs or hypervariable loops from heavy and light chain variable regions having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions).

In certain embodiments, the nucleic acid can comprise a nucleotide sequence encoding at least one, two, or three CDRs or hypervariable loops from a heavy chain variable region having the nucleotide sequence as set forth in the tables herein, a sequence substantially homologous thereto (e.g., a sequence at least about 85%, 90%, 95%, 99% or more identical thereto, and/or capable of hybridizing under the stringency conditions described herein). In another embodiment, the nucleic acid can comprise a nucleotide sequence encoding at least one, two, or three CDRs or hypervariable loops from a light chain variable region having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein). In yet another embodiment, the nucleic acid can comprise a nucleotide sequence encoding at least one, two, three, four, five, or six CDRs or hypervariable loops from heavy and light chain variable regions having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein).

In certain embodiments, the nucleic acid can comprise a nucleotide sequence encoding a cytokine molecule, an immune cell engager, or a stromal modifying moiety as described herein.

In another aspect, the application features host cells and vectors containing the nucleic acids described herein. The nucleic acids may be present in a single vector or separate vectors present in the same host cell or separate host cell, as described in more detail hereinbelow.

### Vectors

Described herein, in certain embodiments, is a vector comprising one or more of the nucleic acid molecules as described herein.

Further provided herein are vectors comprising the nucleotide sequences encoding antibody molecules, e.g., anti-TCRβV antibody molecules, anti-TCRαV antibody molecules, or a multispecific or multifunctional molecule described herein. In some embodiments, the vectors comprise nucleic acid sequences encoding antibody molecules, e.g., anti-TCRαV antibody molecules, anti-TCRαV antibody molecules, or multispecific or multifunctional molecule described herein. In some embodiments, the vectors comprise the nucleotide sequences described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity.

Methods and conditions for culturing the resulting transfected cells and for recovering the antibody molecule produced are known to those skilled in the art, and may be varied or optimized depending upon the specific expression vector and mammalian host cell employed, based upon the present description.

### Cells

Described herein, in certain embodiments, is a cell comprising the nucleic acid as described herein or the vector as described herein.

In another aspect, described herein are host cells and vectors containing the nucleic acids. The nucleic acids may be present in a single vector or separate vectors present in the same host cell or separate host cell. The host cell can be a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell.

In some embodiments, described herein are host cells comprising a nucleic acid encoding an antibody molecule as described herein.

In some embodiments, described herein are the host cells genetically engineered to comprise nucleic acids encoding the antibody molecule.

In some embodiments, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

In some embodiments, described herein are host cells comprising the vectors described herein. The cell can be, but is not limited to, a eukaryotic cell, a bacterial cell, an insect cell, or a human cell. Suitable eukaryotic cells include, but are not limited to. Vero cells, HeLa cells, COS cells, CHO cells, HEK293 cells, BHK cells and MDCKII cells. Suitable insect cells include, but are not limited to, Sf9 cells.

Method of Expanding Cells with Anti-TCRVB Antibodies or Anti-TCRVA Antibodies

Any of the compositions and methods described herein can be used to expand an immune cell population. An immune cell provided herein includes an immune cell derived from a hematopoietic stem cell or an immune cell derived from a non-hematopoietic stem cell, e.g., by differentiation or de-differentiation.

An immune cell includes a hematopoietic stem cell, progeny thereof and/or cells that have differentiated from said HSC, e.g., lymphoid cells or myeloid cells. An immune cell can be an adaptive immune cell or an innate immune cell. Examples of immune cells include T cells, B cells, Natural Killer cells, Natural Killer T cells, neutrophils, dendritic cells, monocytes, macrophages, and granulocytes.

In some embodiments, an immune cell is a T cell. In some embodiments, a T cell includes a CD4+ T cell, a CD8+ T cell, a TCR alpha-beta T cell, a TCR gamma-delta T cell. In some embodiments, a T cell comprises a memory T cell (e.g., a central memory T cell, or an effector memory T cell (e.g., a TEMRA) or an effector T cell. In some embodiments, a T cell comprises a tumor infiltrating lymphocyte (TIL).

In some embodiments, an immune cell is an NK cell.

In some embodiments, an immune cell is a TIL. TILs are immune cells (e.g., T cells, B cells or NK cells) that can be found in a tumor or around a tumor (e.g., in the stroma or tumor microenvironment of a tumor). e.g., a solid tumor, e.g., as described herein. TILs can be obtained from a sample from a subject having cancer, e.g., a biopsy or a surgical sample. In some embodiments, TILs can be expanded using a method as described herein. In some embodiments, a population of expanded TILs, e.g., expanded using a method as described herein, can be administered to a subject to treat a disease, e.g., a cancer.

In some embodiments, immune cells, e.g., T cells (e.g., TILs), can be obtained from a unit of blood collected from a subject using any number of techniques known to the skilled artisan, such as Ficoll™ separation. In one aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and, optionally, to place the cells in an appropriate buffer or media for subsequent processing steps. In some embodiments, the cells are washed with phosphate buffered saline (PBS). In an alternative embodiment, the wash solution lacks calcium and may lack magnesium or may lack many if not all divalent cations. The methods described herein can include more than one selection step, e.g., more than one depletion step.

In some embodiments, the methods of the application can utilize culture media conditions comprising DMEM, DMEM F12, RPMI 1640, and/or AIM V media. The media can be supplemented with glutamine, HEPES buffer (e.g., 10 mM), serum (e.g., heat-inactivated serum, e.g., 10%), and/or beta mercaptoethanol (e.g., 55 uM). IN some embodiments, the culture conditions as described herein comprise one or more supplements, cytokines, growth factors, or hormones. In some embodiments, the culture condition comprises one or more of IL-2, IL-15, or IL-7, or a combination thereof.

Immune effector cells such as T cells may be activated and expanded generally using methods as described, for example, in U.S. Pat. No. 6,352,694; 6,534,055; or 6,905,680. Generally, a population of immune cells, may be expanded by contact with an agent that stimulates a CD3/TCR complex associated signal and a ligand that stimulates a costimulatory molecule on the surface of the T cells; and/or by contact with a cytokine, e.g., IL-2, IL-15 or IL-7. T cell expansion protocols can also include stimulation, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besaneon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

A TIL population can also be expanded by methods known in the art. For example, a population of TILs can be expanded as described in Hall et al., Journal for ImmunoTherapy of Cancer (2016) 4:61, the entire contents of which are hereby incorporated by reference. Briefly, TILs can be isolated from a sample by mechanical and/or physical digestion. The resultant TIL population can be stimulated with an anti-CD3 antibody in the presence of non-dividing feeder cells. In some embodiments, the TIL population can be cultured, e.g., expanded, in the presence of IL-2, e.g., human IL-2. In some embodiments, the TIL cells can be cultured, e.g., expanded for a period of at least 1-21 days, e.g., at least 1, 2, 3, 4, 5, 6, 7, 8, 9, 10, 11, 12, 13, 14, 15, 16, 17, 18, 19, 20 or 21 days.

As described herein, in some embodiments, an immune cell population (e.g., a T cell (e.g., a T_{EMRA} cell or a TIL population) can be expanded by contacting the immune cell population with an anti-TCRVB antibody or an anti-TCRVA antibody. e.g., as described herein.

In some embodiments, the expansion occurs in vivo, e.g., in a subject.

In some embodiments, a subject is administered the multispecific or multifunctional molecules comprising TCRβV-binding moieties as described herein resulting in expansion of immune cells in vivo.

In some embodiments, a subject is administered the multispecific or multifunctional molecules comprising TCRαV-binding moieties as described herein resulting in expansion of immune cells in vivo.

In some embodiments, the expansion occurs ex vivo, e.g., in vitro. In some embodiments, cells from a subject, e.g., T cells, e.g., TIL cells, are expanded in vitro with the multispecific or multifunctional molecules as described herein. In some embodiments, the expanded TILs are administered to the subject to treat a disease or a symptom of a disease.

In some embodiments, a method of expansion as described herein results in an expansion of at least 1.1-10 fold, 10-20 fold, or 20-50 fold expansion. In some embodiments, the expansion is at least 1.1, 1.2, 1.3, 1.4, 1.5, 2, 3, 4, 5, 6, 7, 8, 9, 10, 15, 20, 25, 30, 35, 40, 45 or 50 fold expansion.

In some embodiments, a method of expansion as described herein comprises culturing, e.g., expanding, the cells for at least about 4 hours, 6 hours, 10 hours, 12 hours, 15 hours, 18 hours, 20 hours, or 22 hours. In some embodiments, a method of expansion as described herein comprises culturing, e.g., expanding, the cells for at least 1, 2, 3, 4, 5, 6, 7, 8, 9, 10, 11, 12, 13, 14, 15, 16 17, 18, 19, 20 or 21 days. In some embodiments, a method of expansion as described herein comprises culturing. e.g., expanding, the cells for at least about 1 week, 2 weeks, 3 weeks, 4 weeks, 5 weeks, 6 weeks, 7 weeks or 8 weeks.

In some embodiments, a method of expansion as described herein is performed on immune cells obtained from a healthy subject.

In some embodiments, a method of expansion as described herein is performed on immune cells (e.g., TILs) obtained from a subject having a disease, e.g., a cancer, e.g., a solid tumor as described herein.

In some embodiments, a method of expansion as described herein further comprises contacting the population of cells with an agent, that promotes, e.g., increases, immune cell expansion. In some embodiments, the agent comprises an immune checkpoint inhibitor, e.g., a PD-1 inhibitor, a LAG-3 inhibitor, a CTLA4 inhibitor, or a TIM-3 inhibitor. In some embodiments, the agent comprises a 4-1BB agonist, e.g., an anti-4-1BB antibody.

Without wishing to be bound by theory, in some embodiments, the multispecific or multifunctional molecules as described herein can expand, e.g., selectively or preferentially expand, T cells expressing a T cell receptor (TCR) comprising a TCR alpha and/or TCR beta molecule, e.g., TCR alpha-beta T cells (as T cells). In some embodiments, the multispecific or multifunctional molecules as described herein do not expand, or induce proliferation of T cells expressing a TCR comprising a TCR gamma and/or TCR delta molecule, e.g., TCR gamma-delta T cells (γδ T cells). In some embodiments, the multispecific or multifunctional molecules as described herein selectively or preferentially expand αβ T cells over γδ T cells.

Without wishing to be bound by theory, it is believed that, in some embodiments, γδ T cells are associated with cytokine release syndrome (CRS) and/or neurotoxicity (NT). In some embodiments, the multispecific or multifunctional molecules as described herein result in selective expansion of non-γδ T cells, e.g., expansion of as T cells, thus reducing CRS and/or NT.

In some embodiments, any of the compositions or methods as described herein result in an immune cell population having a reduction of, e.g., depletion of, γδ T cells. In some embodiments, the immune cell population is contacted with an agent that reduces, e.g., inhibits or depletes, γδ T cells.

### CRS Grading

In some embodiments, CRS (Cytokine Release Syndrome) can be graded in severity from 1-5 as follows. Grades 1-3 are less than severe CRS. Grades 4-5 are severe CRS. For Grade 1 CRS, only symptomatic treatment is needed (e.g., nausea, fever, fatigue, myalgias, malaise, headache) and symptoms are not life threatening. For Grade 2 CRS, the symptoms require moderate intervention and generally respond to moderate intervention. Subjects having Grade 2 CRS develop hypotension that is responsive to either fluids or one low-dose vasopressor; or they develop grade 2 organ toxicity or mild respiratory symptoms that are responsive to low flow oxygen (<40% oxygen). In Grade 3 CRS subjects, hypotension generally cannot be reversed by fluid therapy or one low-dose vasopressor. These subjects generally require more than low flow oxygen and have grade 3 organ toxicity (e.g., renal or cardiac dysfunction or coagulopathy) and/or grade 4 transaminitis. Grade 3 CRS subjects require more aggressive intervention, e.g., oxygen of 40% or higher, high dose vasopressor(s), and/or multiple vasopressors. Grade 4 CRS subjects suffer from immediately life-threatening symptoms, including grade 4 organ toxicity or a need for mechanical ventilation. Grade 4 CRS subjects generally do not have transaminitis. In Grade 5 CRS subjects, the toxicity causes death. Sets of criteria for grading CRS are provided herein as Table 5, Table 6, and Table 7. Unless otherwise specified, CRS as used herein refers to CRS according to the criteria of Table 6.

In some embodiments, CRS is graded according to Table 5.

The term “cytokine profile” as used herein, refers to the level and/or activity of on one or more cytokines or chemokines, e.g., as described herein. In some embodiments, a cytokine profile comprises the level and/or activity of a naturally occurring cytokine, a fragment or a functional fragment or a functional variant thereof. In some embodiments, a cytokine profile comprises the level and/or activity of one or more cytokines and/or one or more chemokines (e.g., as described herein). In some embodiments, a cytokine profile comprises the level and/or activity of a naturally occurring cytokine, a fragment or a functional fragment or a functional variant thereof. In some embodiments, a cytokine profile comprises the level and/or activity of a naturally occurring chemokine, a fragment or a functional fragment or a functional variant thereof. In some embodiments, a cytokine profile comprises the level and/or activity of one or more of: IL-2 (e.g., full length, a variant, or a fragment thereof); IL-1beta (e.g., full length, a variant, or a fragment thereof); IL-6 (e.g., full length, a variant, or a fragment thereof); TNFα (e.g., full length, a variant, or a fragment thereof); IFNgamma (e.g., full length, a variant, or a fragment thereof) IL-10 (e.g., full length, a variant, or a fragment thereof); IL-4 (e.g., full length, a variant, or a fragment thereof); TNF alpha (e.g., full length, a variant, or a fragment thereof); IL-12p70 (e.g., full length, a variant, or a fragment thereof); IL-13 (e.g., full length, a variant, or a fragment thereof); IL-8 (e.g., full length, a variant, or a fragment thereof); Eotaxin (e.g., full length, a variant, or a fragment thereof); Eotaxin-3 (e.g., full length, a variant, or a fragment thereof); IL-8 (HA) (e.g., full length, a variant, or a fragment thereof); IP-10 (e.g., full length, a variant, or a fragment thereof); MCP-1 (e.g., full length, a variant, or a fragment thereof); MCP-4 (e.g., full length, a variant, or a fragment thereof); MDC (e.g., full length, a variant, or a fragment thereof); MIP-1α(e.g., full length, a variant, or a fragment thereof); MIP-1b (e.g., full length, a variant, or a fragment thereof); TARC (e.g., full length, a variant, or a fragment thereof); GM-CSF (e.g., full length, a variant, or a fragment thereof); IL-12 23p40 (e.g., full length, a variant, or a fragment thereof); IL-15 (e.g., full length, a variant, or a fragment thereof); IL-16 (e.g., full length, a variant, or a fragment thereof); IL-17a (e.g., full length, a variant, or a fragment thereof); IL-1a (e.g., full length, a variant, or a fragment thereof); IL-5 (e.g., full length, a variant, or a fragment thereof); IL-7 (e.g., full length, a variant, or a fragment thereof); TNF-beta (e.g., full length, a variant, or a fragment thereof); or VEGF (e.g., full length, a variant, or a fragment thereof). In some embodiments, a cytokine profile includes secretion of one or more cytokines or chemokines. In some embodiments, a cytokine in a cytokine profile can be modulated, e.g., increased or decreased, by an anti-TCRβV antibody or an anti-TCRAV antibody molecule described herein. In some embodiments, the cytokine profile includes cytokines associated with a cytokine storm or cytokine release syndrome (CRS), e.g., IL-6, IL-1beta, TNFalpha and IL-10.

### Pharmaceutical Compositions

Described herein, in certain embodiments, is a pharmaceutical composition comprising the multifunctional polypeptide molecule as described herein, the nucleic acid molecules as described herein, the vector as described herein, or the cell as described herein, and a pharmaceutically acceptable carrier, excipient, or diluent.

Pharmaceutical compositions or formulations comprising the agent, e.g., the multifunctional or multispecific molecules, of the described compositions and for use in any of the described methods can be prepared according to conventional techniques well known in the pharmaceutical industry and described in the published literature. In some embodiments, a pharmaceutical composition or formulation for treating a subject comprises an effective amount of any the multifunctional or multispecific molecules or the compositions as described herein, or a pharmaceutically acceptable salt, solvate, hydrate or ester thereof. The pharmaceutical formulation comprising the multifunctional or multispecific molecules as described herein may further comprise a pharmaceutically acceptable excipient, diluent or carrier.

Pharmaceutically acceptable salts are suitable for use in contact with the tissues of humans and lower animals without undue toxicity, irritation, allergic response, etc., and are commensurate with a reasonable benefit/risk ratio. (See, e.g., S. M. Berge, et al., J. Pharmaceutical Sciences, 66: 1-19 (1977), incorporated herein by reference for this purpose. The salts can be prepared in situ during the final isolation and purification of the compounds, or separately by reacting the free base form with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

In some embodiments, the compositions are formulated into any of many possible dosage forms such as, but not limited to, tablets, capsules, gel capsules, liquid syrups, soft gels, suppositories, and enemas. In some embodiments, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. In some embodiments, a pharmaceutical formulation or composition as described herein includes, but is not limited to, a solution, emulsion, microemulsion, foam or liposome-containing formulation (e.g., cationic or noncationic liposomes).

The pharmaceutical composition or formulation described herein may comprise one or more penetration enhancers, carriers, excipients or other active or inactive ingredients as appropriate and well known to those of skill in the art or described in the published literature. In some embodiments, liposomes also include sterically stabilized liposomes, e.g., liposomes comprising one or more specialized lipids. These specialized lipids result in liposomes with enhanced circulation lifetimes. In some embodiments, a sterically stabilized liposome comprises one or more glycolipids or is derivatized with one or more hydrophilic polymers, such as a polyethylene glycol (PEG) moiety. In some embodiments, a surfactant is included in the pharmaceutical formulation or compositions. The use of surfactants in drug products, formulations and emulsions is well known in the art. In some embodiments, the present disclosure employs a penetration enhancer to effect the efficient delivery of the multifunctional or multispecific molecules or the compositions as described herein, e.g., to aid diffusion across cell membranes and/or enhance the permeability of a lipophilic drug. In some embodiments, the penetration enhancers are a surfactant, fatty acid, bile salt, chelating agent, or non-chelating nonsurfactant.

In some embodiments, the pharmaceutical formulation comprises multiple multifunctional or multispecific molecules as described herein. In some embodiments, the multifunctional or multispecific molecules or the compositions as described herein is administered in combination with another drug or therapeutic agent.

### Treatment of Subjects

Any of the compositions provided herein may be administered to an individual. “Individual” may be used interchangeably with “subject” or “patient.” An individual may be a mammal, for example a human or animal such as a non-human primate, a rodent, a rabbit, a rat, a mouse, a horse, a donkey, a goat, a cat, a dog, a cow, a pig, or a sheep. In some embodiments, the individual is a human. In some embodiments, the individual is a fetus, an embryo, or a child. In other embodiments, the individual may be another eukaryotic organism, such as a plant. In some embodiments, the compositions provided herein are administered to a cell ex vivo.

In some embodiments, the compositions provided herein are administered to an individual as a method of treating a disease or disorder. In some embodiments, the individual has a genetic disease, such as any of the diseases described herein. In some embodiments, the individual is at risk of having a disease, such as any of the diseases described herein. In some embodiments, the individual is at increased risk of having a disease or disorder caused by insufficient amount of a protein or insufficient activity of a protein. If an individual is “at an increased risk” of having a disease or disorder caused insufficient amount of a protein or insufficient activity of a protein, the method involves preventative or prophylactic treatment. For example, an individual may be at an increased risk of having such a disease or disorder because of family history of the disease. Typically, individuals at an increased risk of having such a disease or disorder benefit from prophylactic treatment (e.g., by preventing or delaying the onset or progression of the disease or disorder). In some embodiments, a fetus is treated in utero, e.g., by administering the multifunctional or multispecific molecules or the compositions as described herein to the fetus directly or indirectly (e.g., via the mother).

Suitable routes for administration of the multifunctional or multispecific molecules or the compositions as described herein may vary depending on cell type to which delivery of the multifunctional or multispecific molecules or the compositions is desired. The multifunctional or multispecific molecules or the compositions as described herein may be administered to patients parenterally, for example, by intrathecal injection, intracerebroventricular injection, intraperitoneal injection, intramuscular injection, subcutaneous injection, or intravenous injection. The multifunctional or multispecific molecules or the compositions as described herein may be administered to patients orally.

In some embodiments, the multifunctional or multispecific molecules or the compositions as described herein are administered with one or more agents capable of promoting penetration of the subject the multifunctional or multispecific molecules or the compositions as described herein across the blood-brain barrier by any method known in the art. For example, delivery of agents by administration of an adenovirus vector to motor neurons in muscle tissue is described in U.S. Pat. No. 6,632,427, “Adenoviral-vector-mediated gene transfer into medullary motor neurons,” incorporated herein by reference. Delivery of vectors directly to the brain, e.g., the striatum, the thalamus, the hippocampus, or the substantia nigra, is described, e.g., in U.S. Pat. No. 6,756,523, “Adenovirus vectors for the transfer of foreign genes into cells of the central nervous system particularly in brain,” incorporated herein by reference.

In some embodiments, the multifunctional or multispecific molecules or the compositions as described herein are linked or conjugated with agents that provide desirable pharmaceutical or pharmacodynamic properties. In some embodiments, the multifunctional or multispecific molecules or the compositions as described herein are coupled to a substance, known in the art to promote penetration or transport across the blood-brain barrier, e.g., an antibody to the transferrin receptor. In some embodiments, the multifunctional or multispecific molecules or the compositions as described herein are linked with a viral vector.

In some embodiments, subjects treated using the methods and compositions are evaluated for improvement in condition using any methods known and described in the art.

The terms “treat,” “treating”, and “treatment,” and the like are used herein to generally mean obtaining a desired pharmacological and/or physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly, a human, and includes: (a) preventing the disease from occurring in a subject which may be predisposed to the disease but has not yet been diagnosed as having it; (b) inhibiting the disease, i.e., arresting its development; or (c) relieving the disease, i.e., mitigating or ameliorating the disease and/or its symptoms or conditions. The term “prophylaxis” is used herein to refer to a measure or measures taken for the prevention or partial prevention of a disease or condition. In some embodiments, the terms “condition,” “disease.” or “disorder,” as used herein, are interchangeable.

By “treating or preventing a disease or a disorder” is meant ameliorating any of the conditions or signs or symptoms associated with the disorder before or after it has occurred. As compared with an equivalent untreated control, such reduction or degree of prevention is at least 3%, 5%, 10%, 20%, 40%, 50%, 60%, 80%, 90%, 95%, or 100% as measured by any standard technique. A patient who is being treated for a disease or a disorder, is one who a medical practitioner has diagnosed as having such a condition. Diagnosis may be by any suitable means. Diagnosis and monitoring may involve, for example, detecting the presence of pathological cells in a biological sample (e.g., tissue biopsy, blood test, or urine test), detecting the level of a surrogate marker of the disorder in a biological sample, or detecting symptoms associated with the disorder. A patient in whom the development of a disorder is being prevented may or may not have received such a diagnosis. One in the art will understand that these patients may have been subjected to the same standard tests as described above or may have been identified, without examination, as one at high risk due to the presence of one or more risk factors (e.g., family history or genetic predisposition).

### Methods of Cancer Treatment

Described herein, in certain embodiments, is a method of treating a condition or disease in a subject in need therefor comprising administering to the subject a therapeutically effective amount of the multifunctional polypeptide molecule as described herein, the nucleic acid molecules as described herein, the vector as described herein, the cell as described herein, the pharmaceutical composition as described herein, or a combination thereof, wherein the administering is effective to treat the condition or disease in the subject.

In some embodiments, the condition or disease is cancer. In some embodiments, the cancer is a solid tumor, a hematological cancer, a metastatic cancer, a soft tissue tumor, or a combination thereof. In some embodiments, the cancer is the solid tumor, and wherein the solid tumor is selected from the group consisting of melanoma, pancreatic cancer, breast cancer, colorectal cancer, lung cancer, skin cancer, ovarian cancer, liver cancer, and a combination thereof. In some embodiments, the cancer is the hematological cancer, and wherein the hematological cancer is selected from the group consisting of Hodgkin's lymphoma, Non-Hodgkin's lymphoma, acute myeloid leukemia (AML), chronic myeloid leukemia, myelodysplastic syndrome, multiple myeloma, T-cell lymphoma, acute lymphocytic leukemia, and a combination thereof. In some embodiments, the Non-Hodgkin's lymphoma is selected from the group consisting of B cell lymphoma, diffuse large B cell lymphoma (DLBCL), follicular lymphoma, chronic lymphocytic leukemia (B-CLL), mantle cell lymphoma, marginal zone B-cell lymphoma, Burkitt lymphoma, lymphoplasmacytic lymphoma, hairy cell leukemia, and a combination thereof. In some embodiments, the T-cell lymphoma is peripheral T-cell lymphoma.

Non-limiting examples of cancers that can be treated with the compounds of the present disclosure include cancer cells from the bladder, blood, bone, bone marrow, brain, breast, colon, esophagus, gastrointestine, gum, head, kidney, liver, lung, nasopharynx, neck, ovary, prostate, skin, stomach, pancreas, prostate testis, tongue, cervix, or uterus. Non-limiting examples of cancer histological types include neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In some embodiments, the tumor may comprise an osteosarcoma, angiosarcoma, rhabdosarcoma, leiomyosarcoma, Ewing sarcoma, glioblastoma, neuroblastoma, or leukemia.

In some embodiments, the cancer is characterized by a cancer antigen present on the cancer. In some embodiments, the cancer antigen is a tumor antigen, a stromal antigen, or a hematological antigen. In some embodiments, the cancer antigen is selected from the group consisting of BCMA. CD19, CD20, CD22, CD30, CD33, FcRH5, PDL1, CD47, CD117 (c-kit), gangloside 2(GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, calcitonin, Cyclin-B1, Cyclin D1, DCP, 9D7, Ep-CAM, EphA3, Gastrin. HE4, Her2/neu, Telomerase, SAP-1, Survivin, NY-ESO-1/LAGE-1, PRAME, SSX-2, Melan-A/MART-1, 5-HIAA, Gp100/pmel17, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA15-3, CA27.29, CA19-9, CA-125, BAGE, GAGE, NY-ESO-1, β-catenin, CDK4, CDC27, α actinin-4, TRP1/gp75, TRP2, gp100, lactate dehydrogenase, Melan-A/MART1, gangliosides, WT1, EphA3, Epidermal growth factor receptor (EGFR), MART-2, MART-1, MPO, MUC1, MUC2, MUM1, MUM2, MUM3, NA88-1, nuclear matrix protein 22, NPM, OA1, OGT, PAP, PD-L1, RCC, RU11, RU12, SAGE, TdT, TPMT, TRG, TRP1, TSTA, Folate receptor alpha, L1-CAM, CAIX, gpA33, GD3, GM2, VEGFR, Intergrins, carbohydrates, IGF1R, EPHA3, TRAILR1, TRAILR2, RANKL, FAP, TGF-beta, hyaluronic acid, collagen, tenascin C, and tenascin W.

Methods described herein include treating a cancer in a subject by using multispecific or multifunctional molecules as described herein, e.g., using a pharmaceutical composition described herein. Also provided are methods for reducing or ameliorating a symptom of a cancer in a subject, as well as methods for inhibiting the growth of a cancer and/or killing one or more cancer cells. In some embodiments, the methods described herein decrease the size of a tumor and/or decrease the number of cancer cells in a subject administered with a described herein or a pharmaceutical composition described herein.

Described herein are methods of treating a subject having a cancer comprising acquiring a status of one or more TCRβV molecules in a subject. In some embodiments, a higher, e.g., increased, level or activity of one or more TCRβV molecules in a subject, e.g., in a sample from a subject, is indicative of a bias, e.g., a preferential expansion, e.g., clonal expansion, of T cells expressing said one or more TCRβV molecules in the subject.

Without wishing to be bound by theory, it is believed that a biased T cell population, e.g., a T cell population expressing a TCRβV molecule, is antigen-specific for a disease antigen, e.g., a cancer antigen (Wang C Y, et al., Int J Oncol. (2016) 48(6):2247-56). In some embodiments, the cancer antigen comprises a cancer associated antigen or a neoantigen. In some embodiments, a subject having a cancer, e.g., as described herein, has a higher, e.g., increased, level or activity of one or more TCRβV molecules associated with the cancer. In some embodiments, the TCRβV molecule is associated with, e.g., recognizes, a cancer antigen, e.g., a cancer associated antigen or a neoantigen.

Accordingly, as described herein are methods of expanding an immune effector cell population obtained from a subject, comprising acquiring a status of one or more TCRβV molecules in a sample from the subject, comprising contacting said immune effector cell population with an anti-TCRβV antibody molecule as described herein, e.g., an anti-TCRβV antibody molecule that binds to the same TCRβV molecule that is higher, e.g., increased in the immune effector cell population in the sample from the subject. In some embodiments, contacting the population of immune effector cells (e.g., comprising T cells that express one or more TCRβV molecules) with an anti-TCRβV molecule results in expansion of the population of immune effector cells expressing one or more TCRβV molecules. In some embodiments, the expanded population, or a portion thereof, is administered to the subject (e.g., same subject from whom the immune effector cell population was obtained), to treat the cancer. In some embodiments, the expanded population, or a portion thereof, is administered to a different subject (e.g., not the same subject from whom the immune effector cell population was obtained), to treat the cancer.

Also described herein are methods of treating a subject having a cancer, comprising: acquiring a status of one or more TCRβV molecules in a sample from the subject, and determining whether the one or more TCRβV molecules is higher, e.g., increased, in a sample from the subject compared to a reference value, wherein responsive to said determination, administering to the subject an effective amount of an anti-TCRβV antibody molecule, e.g., an agonistic anti-TCRβV antibody molecule, e.g., as described herein.

In some embodiments, the subject has B-CLL. In some embodiments, a subject having B-CLL has a higher, e.g., increased, level or activity of one or more TCRβV molecules, e.g., one or more TCRβV molecules comprising: (i) TCRβ V6 subfamily comprising, e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01.

In some embodiments, a subject having B-CLL has a higher, e.g., increased, level or activity of a TCRβ V6 subfamily comprising. e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments, the subject is administered the multifunctional polypeptide molecule as described herein comprising an anti-TCRβV molecule (e.g., an agonistic anti-TCRβV molecule as described herein) that binds to one or more members of the TCRβ V6 subfamily. In some embodiments, administration of the multifunctional polypeptide molecule as described herein results in expansion of immune cells expressing one or more members of the TCRβ V6 subfamily.

In some embodiments, the subject has melanoma. In some embodiments, a subject having melanoma has a higher, e.g., increased, level or activity of one or more TCRβV molecules, e.g., one or more TCRβV molecules comprising the TCRβ V6 subfamily comprising, e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments, the subject is administered the multifunctional polypeptide molecule as described herein comprising an anti-TCRβV molecule (e.g., an agonistic anti-TCRβV molecule as described herein) that binds to one or more members of the TCRβ V6 subfamily. In some embodiments, administration of the multifunctional polypeptide molecule as described herein results in expansion of immune cells expressing one or more members of the TCRβ V6 subfamily.

In some embodiments, the subject has CRC (colorectal cancer). In some embodiments, a subject having CRC has a higher, e.g., increased, level or activity of one or more TCRβV molecules, e.g., one or more TCRβV molecules comprising TCRβ V12 subfamily comprising TCRβ V12-4*01, TCRβ V12-3*01, or TCRβ V12-5*01.

In some embodiments, a subject having CRC has a higher, e.g., increased, level or activity of a TCRβ V12 subfamily comprising TCRβ V12-4*01, TCRβ V12-3*01, or TCRβ V12-5*01. In some embodiments, the subject is administered the multifunctional polypeptide molecule as described herein comprising an anti-TCRβV molecule (e.g., an agonistic anti-TCRAV molecule as described herein) that binds to one or more members of the TCRβ V12 subfamily. In some embodiments, administration of the multifunctional polypeptide molecule as described herein results in expansion of immune cells expressing one or more members of the TCRβ V12 subfamily.

In some embodiments, acquiring a value for the status, e.g., presence, level and/or activity, of one or more TCRβV molecules comprises acquiring a measure of the T cell receptor (TCR) repertoire of a sample. In some embodiments, the value comprises a measure of the clonotype of a population of T cells in the sample.

In some embodiments, a value for the status of one or more TCRβV molecules is obtained, e.g., measured, using an assay described in Wang C Y, et al., Int J Oncol. (2016) 48(6):2247-56, the entire contents of which are hereby incorporated by reference.

In some embodiments, a value for the status of one or more TCRβV molecules is obtained, e.g., measured, using flow cytometry.

Described herein are methods of treating a subject having a cancer comprising acquiring a status of one or more TCRAV molecules in a subject. In some embodiments, a higher, e.g., increased, level or activity of one or more TCRαV molecules in a subject, e.g., in a sample from a subject, is indicative of a bias, e.g., a preferential expansion, e.g., clonal expansion, of T cells expressing said one or more TCRαV molecules in the subject.

Without wishing to be bound by theory, it is believed that a biased T cell population, e.g., a T cell population expressing a TCRαV molecule, is antigen-specific for a disease antigen, e.g., a cancer antigen (Wang C Y, et al., Int J Oncol. (2016) 48(6):2247-56). In some embodiments, the cancer antigen comprises a cancer associated antigen or a neoantigen. In some embodiments, a subject having a cancer, e.g., as described herein, has a higher, e.g., increased, level or activity of one or more TCRAV molecules associated with the cancer. In some embodiments, the TCRαV molecule is associated with, e.g., recognizes, a cancer antigen, e.g., a cancer associated antigen or a neoantigen.

Accordingly, as described herein are methods of expanding an immune effector cell population obtained from a subject, comprising acquiring a status of one or more TCRAV molecules in a sample from the subject, comprising contacting said immune effector cell population with an anti-TCRαV antibody molecule as described herein, e.g., an anti-TCRAV antibody molecule that binds to the same TCRαV molecule that is higher, e.g., increased in the immune effector cell population in the sample from the subject. In some embodiments, contacting the population of immune effector cells (e.g., comprising T cells that express one or more TCRαV molecules) with an anti-TCRAV molecule results in expansion of the population of immune effector cells expressing one or more TCRαV molecules. In some embodiments, the expanded population, or a portion thereof, is administered to the subject (e.g., same subject from whom the immune effector cell population was obtained), to treat the cancer. In some embodiments, the expanded population, or a portion thereof, is administered to a different subject (e.g., not the same subject from whom the immune effector cell population was obtained), to treat the cancer.

Also described herein are methods of treating a subject having a cancer, comprising: acquiring a status of one or more TCRαV molecules in a sample from the subject, and determining whether the one or more TCRαV molecules is higher, e.g., increased, in a sample from the subject compared to a reference value, wherein responsive to said determination, administering to the subject an effective amount of an anti-TCRαV antibody molecule, e.g., an agonistic anti-TCRαV antibody molecule, e.g., as described herein.

Described herein are methods of treating a subject having a cancer comprising acquiring a status of one or more TCRAV molecules in a subject. In some embodiments, a higher, e.g., increased, level or activity of one or more TCRαV molecules in a subject, e.g., in a sample from a subject, is indicative of a bias, e.g., a preferential expansion, e.g., clonal expansion, of T cells expressing said one or more TCRαV molecules in the subject.

Without wishing to be bound by theory, it is believed that a biased T cell population, e.g., a T cell population expressing a TCRαV molecule, is antigen-specific for a disease antigen, e.g., a cancer antigen (Wang C Y, et al., Int J Oncol. (2016) 48(6):2247-56). In some embodiments, the cancer antigen comprises a cancer associated antigen or a neoantigen. In some embodiments, a subject having a cancer, e.g., as described herein, has a higher, e.g., increased, level or activity of one or more TCRAV molecules associated with the cancer. In some embodiments, the TCRαV molecule is associated with, e.g., recognizes, a cancer antigen, e.g., a cancer associated antigen or a neoantigen.

Accordingly, as described herein are methods of expanding an immune effector cell population obtained from a subject, comprising acquiring a status of one or more TCRαV molecules in a sample from the subject, comprising contacting said immune effector cell population with an anti-TCRαV antibody molecule as described herein, e.g., an anti-TCRαV antibody molecule that binds to the same TCRβV molecule that is higher, e.g., increased in the immune effector cell population in the sample from the subject. In some embodiments, contacting the population of immune effector cells (e.g., comprising T cells that express one or more TCRαV molecules) with an anti-TCRAV molecule results in expansion of the population of immune effector cells expressing one or more TCRαV molecules. In some embodiments, the expanded population, or a portion thereof, is administered to the subject (e.g., same subject from whom the immune effector cell population was obtained), to treat the cancer. In some embodiments, the expanded population, or a portion thereof, is administered to a different subject (e.g., not the same subject from whom the immune effector cell population was obtained), to treat the cancer.

Also described herein are methods of treating a subject having a cancer, comprising: acquiring a status of one or more TCRαV molecules in a sample from the subject, and determining whether the one or more TCRαV molecules is higher, e.g., increased, in a sample from the subject compared to a reference value, wherein responsive to said determination, administering to the subject an effective amount of an anti-TCRαV antibody molecule, e.g., an agonistic anti-TCRαV antibody molecule, e.g., as described herein.

### Combination Therapies

In some embodiments, the method described herein comprises administering a combination therapy for treating a disease or condition in a subject in need thereof comprising administering a first agent to the subject, and administering a second agent to the subject, thereby treating the disease or condition in the subject.

In some embodiments, the first agent and second agent are administered not concomitantly to the subject.

In some embodiments, the first agent and second agent are administered concomitantly to the subject.

In some embodiments the first agent and the second agent disclosed herein can be administered in any order or concomitantly to the subject. If concomitantly, the first agent and the second agent can be provided in a single, unified form, or in multiple forms, for example, as multiple separate injections or infusions. The first agent and the second agent can be packed together or separately, in a single package or in a plurality of packages. One or all of the first agent and the second agent can be given in multiple doses. If not concomitantly, the timing between the multiple doses can vary.

In some embodiments, the first agent and second agent are administered sequentially to the subject, wherein first agent is administered to the subject at a first administration and the first agent is administered to the subject subsequently at a second administration.

In some embodiments, the combination therapy disclosed herein is more effective to treat the disease or condition in the subject relative to a method in which the first agent is administered to the subject but not the second agent.

In some embodiments, the combination therapy disclosed herein is more effective to treat the disease or condition in the subject relative to a method in which the second agent is administered to the subject but not the first agent.

In some embodiments, the first target agent can comprise a multispecific molecules disclosed herein, comprising one or more of a tumor-targeting moiety, a cytokine molecule, a stromal modifying moiety, or an immune cell engager.

In some embodiments, the second agent can comprise a multispecific molecules disclosed herein, comprising one or more of a tumor-targeting moiety, a cytokine molecule, a stromal modifying moiety, or an immune cell engager.

In some embodiments, the first agent can comprise a multifunctional molecule. In some embodiments, the second agent can comprise a multifunctional molecule.

In some embodiments, the first agent can comprise an antibody that binds to a first target molecule. In some embodiments, the second agent can comprise an antibody that binds to a second target molecule.

In some embodiment, first target molecule and the second target molecule are independently selected from the group consisting of a T cell receptor alpha variable region (TCRαV) and a T cell receptor beta variable region (TCRβV); and the first target molecule and the second target molecule are different.

In some embodiments the first target molecule the first target molecule is a TCRβV molecule, and the second target molecule is a TCRβV molecule, wherein the first TCRβV molecule and the second TCRβV molecule are different.

In some embodiments the first target molecule the first target molecule is a TCRβV molecule, and the second target molecule is the same TCRαV molecule.

In some embodiments the first target molecule the first target molecule is a TCRαV molecule, and the second target molecule is a TCRαV molecule, wherein the first TCRαV molecule and the second TCRαV molecule are different.

In some embodiments the first target molecule the first target molecule is a TCRαV molecule, and the second target molecule is the same TCRαV molecule.

In some embodiments the first target molecule the first target molecule is a TCRβV molecule, and the second target molecule is a TCRαV molecule.

In some embodiments the first target molecule the first target molecule is a TCRαV molecule, and the second target molecule is a TCRαV molecule.

In some embodiments, the first target molecule is a human TCRβV molecule that binds to human TCRβ 6. In some embodiments, the TCRβ V6 subfamily can comprise TCRβ V6-4*01, TCRβ V64*02, TCRβ V6-9*01, TCRβ V6-8*01, TCRβ V6-5*01, TCRβ V6-6*02, TCRβ V6-6*01, TCRβ V6-2*01, TCRβ V6-3*01, TCRβ V6-1*01, or a variant thereof.

In some embodiments, the first target molecule is a human TCRβV molecule that binds to human TCRβ5. In some embodiments, the TCRβ V5 subfamily can comprise TCRβ V5-5*01, TCRβ V5-6*01, TCRβ V5-4*01, TCRβ V5-8*01, TCRβ V5-1*01, or a variant thereof.

In some embodiments, the first target molecule is a human TCRβV molecule that binds to human TCRβ 10. In some embodiments, the TCRβ V10 subfamily can comprise TCRβ V10-1*01, TCRβ V10-1*02, TCRβ V10-3*01 or TCRβ V10-2*01, or a variant thereof.

In some embodiments, the first target molecule is a human TCRβV molecule that binds to human TCRβ12. In some embodiments, the TCRβ V12 subfamily can comprise TCRβ V12-4*01, TCRβ V12-3*01, TCRβ V12-5*01, or a variant thereof.

In some embodiments, the first target molecule is a human TCRβV molecule that binds to human TCRβ315. In some embodiments, the TCRβ V15 subfamily can comprise TCRβ V15-1*01, or a variant thereof.

In some embodiments, the first target molecule is a human TCRβV molecule that binds to human TCRβ 19. In some embodiments, the TCRβ V19 subfamily can comprise TCRβ V19-1*01, TCRβ V19-1*02, or TCRβ V19-1*03, or a variant thereof.

In some embodiments, the first target molecule is a human TCRβV molecule that binds to human TCRβ20. In some embodiments, the TCRβ V20 subfamily can comprise TCRβ V20-1*01, TCRβ V20-1*02, TCRβ V20-1*03, TCRβ V20-1*04, TCRβ V20-1*05, TCRβ V20-1*06, or TCRβ V20-1*07, or a variant thereof.

In some embodiments, the first target molecule is a human TCRβV molecule that binds to human TCRβ28. In some embodiments, the TCRβ V28 subfamily can comprise TCRβ V28-1*01, or a variant thereof.

In some embodiments, the first target molecule is a human TCRαV molecule that binds to human TCRα, e.g., a TCRα V12 subfamily, a TCRα V13 subfamily, a TCRα V19 subfamily, a TCRα V21 subfamily, a TCRα V30 subfamily, as well as family members of said subfamilies, and variants thereof (e.g., a structural or functional variant thereof).

In some embodiments, the first target molecule is a human TCRαV molecule that binds to human TCRα12. In some embodiments, the TCRα V12 subfamily can comprise TCRαV12-1, TCRαV12-2, or TCRαV12-3, or a variant thereof.

In some embodiments, the first target molecule is a human TCRαV molecule that binds to human TCRα13. In some embodiments, the TCRα V13 subfamily can comprise TCRαV13-1 or TCRαV13-2, or a variant thereof.

In some embodiments, the first target molecule is a human TCRαV molecule that binds to human TCRα21. In some embodiments, the TCRα V21 subfamily can comprise TCRαV21-1 or a variant thereof.

In some embodiments, the TCRα V30 subfamily comprises: TCRαVβ0 or a variant thereof.

In some embodiments, the first target molecule is a mouse TCRβV molecule that binds to mouse TCRβ13. In some embodiments, the TCRβ V13 subfamily can comprise TCRβ V13-1, TCRβ V13-2, TCRβ V13-3, or a variant thereof.

In some embodiments, the first target molecule is a mouse TCRβV molecule that binds to mouse TCRβ17. In some embodiments, the TCRβ V28 subfamily can comprise TCRβ V17-1, or a variant thereof.

In some embodiments, the first target molecule is a mouse TCRβV molecule that binds to mouse TCRβ29. In some embodiments, the TCRβ V29 subfamily can comprise TCRβ V29-1, or a variant thereof.

In some embodiments, the first target molecule is a mouse TCRβV molecule that binds to mouse TCRβ15. In some embodiments, the TCRβ V15 subfamily can comprise TCRβ V15-1, or a variant thereof.

In some embodiments, the second target molecule is a human TCRβV molecule that binds to human TCRβ6. In some embodiments, the TCRβ V6 subfamily can comprise TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01, TCRβ V6-1*01, or a variant thereof.

In some embodiments, the second target molecule is a human TCRβV molecule that binds to human TCRβ5. In some embodiments, the TCRβ V5 subfamily can comprise TCRβ V5-5*01, TCRβ V5-6*01, TCRβ V5-4*01, TCRβ V5-8*01, TCRβ V5-1*01, or a variant thereof.

In some embodiments, the second target molecule is a human TCRβV molecule that binds to human TCRβ10. In some embodiments, the TCRβ V10 subfamily can comprise TCRβ V10-1*01, TCRβ V10-1*02, TCRβ V10-3*01 or TCRβ V10-2*01, or a variant thereof.

In some embodiments, the second target molecule is a human TCRβV molecule that binds to human TCRβ 312. In some embodiments, the TCRβ V12 subfamily can comprise TCRβ V12-4*01, TCRβ V12-3*01, TCRβ V12-5*01, or a variant thereof.

In some embodiments, the second target molecule is a human TCRβV molecule that binds to human TCRβ15. In some embodiments, the TCRβ V15 subfamily can comprise TCRβ V15-1*01, or a variant thereof.

In some embodiments, the second target molecule is a human TCRβV molecule that binds to human TCR19. In some embodiments, the TCRβ V19 subfamily can comprise TCRβ V19-1*01, TCRβ V19-1*02, or TCRβ V19-1*03, or a variant thereof.

In some embodiments, the second target molecule is a human TCRβV molecule that binds to human TCRβ 20. In some embodiments, the TCRβ V20 subfamily can comprise TCRβ V20-1*01, TCRβ V20-1*02, TCRβ V20-1*03, TCRβ V20-1*04, TCRβ V20-1*05, TCRβ V20-1*06, or TCRβ V20-1*07, or a variant thereof.

In some embodiments, the second target molecule is a human TCRβV molecule that binds to human TCRβ28. In some embodiments, the TCRβ V28 subfamily can comprise TCRβ V28-1*01, or a variant thereof.

In some embodiments, the second target molecule is a human TCRαV molecule that binds to human TCRα, e.g., a TCRα V12 subfamily, a TCRα V13 subfamily, a TCRα V19 subfamily, a TCRα V21 subfamily, a TCRα V30 subfamily, as well as family members of said subfamilies, and variants thereof (e.g., a structural or functional variant thereof).

In some embodiments, the second target molecule is a human TCRαV molecule that binds to human TCRα12. In some embodiments, the TCRα V12 subfamily can comprise TCRαV12-1, TCRαV12-2, or TCRαV12-3, or a variant thereof.

In some embodiments, the second target molecule is a human TCRαV molecule that binds to human TCRα13. In some embodiments, the TCRα V13 subfamily can comprise TCRαV13-1 or TCRαV13-2, or a variant thereof.

In some embodiments, the second target molecule is a human TCRαV molecule that binds to human TCRα21. In some embodiments, the TCRα V21 subfamily can comprise TCRαV21-1 or a variant thereof.

In some embodiments, the TCRα V30 subfamily comprises: TCRαVβ0 or a variant thereof.

In some embodiments, the second target molecule is a mouse TCRβV molecule that binds to mouse TCRβ13. In some embodiments, the TCRβ V13 subfamily can comprise TCRβ V13-1, TCRβ V13-2, TCRβ V13-3, or a variant thereof.

In some embodiments, the second target molecule is a mouse TCRβV molecule that binds to mouse TCRβ17. In some embodiments, the TCRβ V28 subfamily can comprise TCRβ V17-1, or a variant thereof.

In some embodiments, the second target molecule is a mouse TCRβV molecule that binds to mouse TCRβ29. In some embodiments, the TCRβ V29 subfamily can comprise TCRβ V29-1, or a variant thereof.

In some embodiments, the second target molecule is a mouse TCRβV molecule that binds to mouse TCRβ15. In some embodiments, the TCRβ V15 subfamily can comprise TCRβ V15-1, or a variant thereof.

In some embodiments, the method described herein comprises a kit, wherein the kit comprises the first agent and the second agent.

In some embodiments, the kit comprises a first pharmaceutical composition comprising the first agent, and a second pharmaceutical composition comprising the second agent, wherein the first pharmaceutical composition and the second pharmaceutical composition independently further comprises a pharmaceutically acceptable excipient, carrier or diluent.

### Administration

In some embodiments, the combination therapy can be administered as soon as is practical after the onset of a disease or condition is detected or suspected, and for a length of time necessary for the treatment of the disease.

In some embodiments, the administration of the first agent can be one or more doses. In some embodiments, the administration of the first agent can be two or more doses. In some embodiments, the administration of the first agent can be 1 dose, 2 doses, 3 doses, 4 doses, 5 doses, 6 doses, 7 doses, 8 doses, 9 doses, 10 doses, 11 doses, 12 doses, 13 doses, 14 doses, 15 doses, 16 doses, 17 doses, 18 doses, 19 doses, or 20 doses.

In some embodiments, the length of time the first agent can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years, about 21 years, about 22 years, about 23 years, about 24 years, or about 25 years.

In some embodiments, the first agent described herein can be administered at any interval desired. The administration of the first agent can have regular or irregular dosing schedules to accommodate either the person administering the agent or the subject receiving the agent. For example, the first agent can be administered, once a day, twice a day, three times a day, once a week, twice a week, three times a week, four times a week, five times a week, once every two weeks, once every three weeks, once every four weeks, once every five weeks, once every six weeks, once every eight weeks, once every ten weeks, once every twelve weeks, once a month, once every two months, once every three months, once every four months, once every six months, once a year, or less frequently. In some embodiments, administration is every other week.

The amount of the first agent administered can be of the same amount in each dose or the dosage can vary between doses. For example, a first amount can be administered in the morning and a second amount can be administered in the evening.

In some embodiments, the administration of the second agent can be one or more doses. In some embodiments, the administration of the second agent can be two or more doses. In some embodiments, the administration of the second agent can be 1 dose, 2 doses, 3 doses, 4 doses, 5 doses, 6 doses, 7 doses, 8 doses, 9 doses, 10 doses, 11 doses, 12 doses, 13 doses, 14 doses, 15 doses, 16 doses, 17 doses, 18 doses, 19 doses, or 20 doses.

In some embodiments, the length of time the second agent can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years, about 21 years, about 22 years, about 23 years, about 24 years, or about 25 years.

In some embodiments, the second agent described herein can be administered at any interval desired. The administration of the second agent can have regular or irregular dosing schedules to accommodate either the person administering the agent or the subject receiving the agent. For example, the second agent can be administered, once a day, twice a day, three times a day, once a week, twice a week, three times a week, four times a week, five times a week, once every two weeks, once every three weeks, once every four weeks, once every five weeks, once every six weeks, once every eight weeks, once every ten weeks, once every twelve weeks, once a month, once every two months, once every three months, once every four months, once every six months, once a year, or less frequently. In some embodiments, administration is every other week.

The amount of the second agent administered can be of the same amount in each dose or the dosage can vary between doses. For example, a first amount can be administered in the morning and a second amount can be administered in the evening.

In some embodiments, the amount of the first agent administered can be the same as the amount of the second agent administered. In some embodiments, the amount of the first agent administered can be different from the amount of the second agent administered.

In some embodiments, the second agent can be administered about 1 hour, about 2 hours, about 3 hours, about 6 hours, about 12 hours, about 24 hours, about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, or about 6 months after the last administration of first agent.

In some embodiments, the dosing regimen comprises the administration of the first and second agent described herein.

In some embodiments, the dosing regimen can be an administration of the first agent once every week for two weeks followed by an administration of the second agent once a week for two weeks, wherein the second agent is administered to the subject one week after the last administration of the first agent.

In some embodiments, the dosing regimen can be an administration of the first agent once every week for three weeks followed by an administration of the second agent once a week for three weeks, wherein the second agent is administered to the subject one week after the last administration of the first agent.

In some embodiments, the dosing regimen can be an administration of the first agent once every week for four weeks followed by an administration of the second agent once a week for four weeks, wherein the second agent is administered to the subject one week after the last administration of the first agent.

In some embodiments, the dosing regimen is repeated once, twice, three times, four times, five times, six times, seven times, eight times, nine times, or ten times.

In some embodiments, the dosing regimen is repeated at the interval of about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years, about 21 years, about 22 years, about 23 years, about 24 years, or about 25 years.

The length of treatment of the dosing regimen can vary for each subject. Amounts effective for this use can vary based on the severity and course of the disease or condition, previous therapy, the subject's health status, weight, and response to the drugs, and the judgment of the treating physician.

In some embodiments, the combination therapy disclosed herein can be administered in a therapeutically-effective amount by various forms and routes including, for example, parenteral, intravenous injection, intravenous infusion, subcutaneous injection, subcutaneous infusion, intramuscular injection, intramuscular infusion, intradermal injection, intradermal infusion, intraperitoneal injection, intraperitoneal infusion, intracerebral injection, intracerebral infusion, subarachnoid injection, subarachnoid infusion, intraocular injection, intraspinal injection, intrastemal injection, endothelial administration, local administration, intranasal administration, intrapulmonary administration, rectal administration, intraarterial administration, intrathecal administration, inhalation, intralesional administration, intradermal administration, epidural administration, absorption through epithelial or mucocutaneous linings (e.g., oral mucosa, rectal and intestinal mucosa), intracapsular administration, subcapsular administration, intracardiac administration, transtracheal administration, subcuticular administration, subarachnoid administration, subcapsular administration, intraspinal administration, or intrasternal administration.

In some embodiments, the combination therapy can be administered in a local manner, for example, via injection of the therapeutic complex directly into an organ, optionally in a depot or sustained release formulation or implant. A pharmaceutical composition can be provided in the form of a rapid release formulation, in the form of an extended-release formulation, or in the form of an intermediate release formulation. A rapid release form can provide an immediate release. An extended-release formulation can provide a controlled release or a sustained delayed release.

In some embodiments, the combination therapy can be administered before, during, or after the occurrence of a disease or condition, and the timing of administering the composition containing a therapeutic complex can vary. For example, a therapeutic complex can be used as a prophylactic and can be administered continuously to subjects with a propensity to conditions or diseases in order to lessen or reduce a likelihood of the occurrence of the disease or condition. A therapeutic complex and composition can be administered to a subject during or as soon as possible after the onset of the symptoms. The administration of a therapeutic complex can be initiated within about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, after the onset of the symptoms. The initial administration can be via any route practical, such as by any route described herein using any formulation described herein.

### Dosing

The combination therapy disclosed herein can be administered via subcutaneous or intravenous injection. The volume of an injection can be about 0.1 mL, about 0.2 mL, about 0.3 mL, about 0.4 mL, about 0.5 mL, about 0.6 mL, about 0.7 mL, about 0.8 mL, about 0.9 mL, about 1 mL, about 1.1 mL, about 1.2 mL, about 1.3 mL, about 1.4 mL, about 1.5 mL, about 1.6 mL, about 1.7 mL, about 1.8 mL, about 1.9 mL, about 2 mL, about 2.1 mL, about 2.2 mL, about 2.3 mL, about 2.4 mL, about 2.5 mL, about 2.6 mL, about 2.7 mL, about 2.8 mL, about 2.9 mL, or about 3 mL.

The combination therapy disclosed herein can be administered at a dosage of about 0.0001 mg/kg to about 1000 mg/kg, about 0.001 mg/kg to about 100 mg/kg, about 0.01 mg/kg to about 100 mg/kg, about 0.01 mg/kg to about 20 mg/kg, about 0.02 mg/kg to about 7 mg/kg, about 0.03 mg/kg to about 5 mg/kg, about 0.05 mg/kg to about 3 mg/kg, about 0.1 mg/kg to about 50 mg/kg, about 0.1 mg/kg to about 0.5 mg/kg, about 0.2 mg/kg to about 0.6 mg/kg, about 0.3 mg/kg to about 0.7 mg/kg, about 0.4 mg/kg to about 0.8 mg/kg, about 0.1 mg/kg to about 0.9 mg/kg, about 0.01 mg/kg to about 50 mg/kg, about 0.1 mg/kg to about 10 mg/kg, about 1 mg/kg to about 10 mg/kg, about 5 mg/kg to about 10 mg/kg, about 1 mg/kg to about 5 mg/kg, or about 3 mg/kg to about 7 mg/kg by mass of the subject.

A therapeutic complex described herein can be administered in any amount necessary or convenient. For example, a therapeutic complex described herein can be administered in an amount from about 0.05 mg to about 300 mg, about 0.1 mg to about 300 mg, about 0.1 mg to about 200 mg, about 0.1 mg to about 100 mg, about 0.05 mg to about 1.5 mg, about 0.1 mg to about 1.5 mg, about 0.05 mg to about 1 mg, about 1 mg to about 1.5 mg, about 0.5 mg to about 6 mg, about 1 mg to about 4 mg, about 2 mg to about 10 mg, about 10 mg to about 30 mg, about 30 mg to about 50 mg, about 50 mg to about 70 mg, about 70 mg to about 100 mg, or about 0.1 mg to about 1 mg, about 0.05 mg, about 0.06 mg, about 0.07 mg, about 0.08 mg, about 0.09 mg, about 0.1 mg, about 0.11 mg, about 0.12 mg, about 0.13 mg, about 0.14 mg, about 0.15 mg, about 0.16 mg, about 0.17, mg, about 0.18 mg, about 0.19 mg, about 0.2 mg, about 0.21 mg, about 0.22 mg, about 0.23 mg, about 0.24 mg, about 0.25 mg, about 0.26 mg, about 0.27, mg, about 0.28 mg, about 0.29 mg, about 0.3 mg, about 0.31 mg, about 0.32 mg, about 0.33 mg, about 0.34 mg, about 0.35 mg, about 0.36 mg, about 0.37, mg, about 0.38 mg, about 0.39 mg, about 0.4 mg, about 0.41 mg, about 0.42 mg, about 0.43 mg, about 0.44 mg, about 0.45 mg, about 0.46 mg, about 0.47, mg, about 0.48 mg, about 0.49 mg, about 0.5 mg, about 0.51 mg, about 0.52 mg, about 0.53 mg, about 0.54 mg, about 0.55 mg, about 0.56 mg, about 0.57, mg, about 0.58 mg, about 0.59 mg, about 0.6 mg, about 0.61 mg, about 0.62 mg, about 0.63 mg, about 0.64 mg, about 0.65 mg, about 0.66 mg, about 0.67, mg, about 0.68 mg, about 0.69 mg, about 0.7 mg, about 0.71 mg, about 0.72 mg, about 0.73 mg, about 0.74 mg, about 0.75 mg, about 0.76 mg, about 0.77, mg, about 0.78 mg, about 0.79 mg, about 0.8 mg, about 0.81 mg, about 0.82 mg, about 0.83 mg, about 0.84 mg, about 0.85 mg, about 0.86 mg, about 0.87, mg, about 0.88 mg, about 0.89 mg, about 0.9 mg, about 0.91 mg, about 0.92 mg, about 0.93 mg, about 0.94 mg, about 0.95 mg, about 0.96 mg, about 0.97, mg, about 0.98 mg, about 0.99 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg about 200 mg, about 210 mg, about 220 mg, about 230 mg, about 240 mg, about 250 mg, about 260 mg, about 270 mg, about 280 mg, about 290 mg, or about 300 mg per dose for a subject by any route of administration.

The combination therapy disclosed herein can be administered at a dosage of about 0.0001 mg/kg, about 0.001 mg/kg, about 0.01 mg/kg, about 0.02 mg/kg, about 0.03 mg/kg, 0.04 mg/kg, about 0.05 mg/kg, 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.5 mg/kg, about 3.0 mg/kg, about 3.5 mg/kg, about 4.0 mg/kg, about 4.5 mg/kg, about 5.0 mg/kg, about 5.5 mg/kg, about 6.0 mg/kg, about 6.5 mg/kg, about 7.0 mg/kg, about 7.5 mg/kg, about 8.0 mg/kg, about 8.5 mg/kg, about 9.0 mg/kg, about 9.5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 110 mg/kg, about 120 mg/kg, about 130 mg/kg, about 140 mg/kg, about 150 mg/kg, about 160 mg/kg, about 170 mg/kg, about 180 mg/kg, about 190 mg/kg, about 200 mg/kg, about 250 mg/kg, about 300 mg/kg, about 350 mg/kg, about 400 mg/kg, about 450 mg/kg, about 500 mg/kg, about 550 mg/kg, about 600 mg/kg, about 650 mg/kg, about 700 mg/kg, about 750 mg/kg, about 800 mg/kg, about 850 mg/kg, about 900 mg/kg, or about 950 mg/kg by mass of the subject.

The combination therapy disclosed herein can be administered at a dosage of from about 0.3 mg/kg to about 1 mg/kg, from about 0.1 mg/kg to about 3.0 mg/kg, from about 0.05 mg/kg to about 5 mg/kg, from about 0.01 mg/kg to about 10 mg/kg, or from about 0.001 mg/kg to about 20 mg/kg by mass of the subject.

Pharmaceutical compositions described herein can be in unit dosage forms suitable for single administration of precise dosages. In unit dosage form, the formulation is divided into unit doses containing appropriate quantities of one or more the therapeutic complexes. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for parenteral injection can be presented in unit dosage form, for example, in ampoules, or in multi-dose containers with a preservative.

### Combination Therapy

In some embodiments, the method as described herein further comprises administering a third therapeutic agent or therapy to the subject.

In some embodiments, the third therapeutic agent or therapy comprises a chemotherapeutic agent, a biologic agent, a hormonal therapy, radiation, or surgery.

In some embodiments, the third therapeutic agent or therapy is administered in combination with the first agent and second agent as described herein, the nucleic acid molecules as described herein, the vector as described herein, the cell as described herein, the pharmaceutical composition as described herein, sequentially, simultaneously, or concurrently.

The first agent and second agent as described herein can be used in combination with a third therapeutic agent or procedure.

In some embodiments, the first agent and second agent as described herein and the third therapeutic agent or procedure are administered/performed after a subject has been diagnosed with a cancer, e.g., before the cancer has been eliminated from the subject. In some embodiments, the multispecific or multifunctional molecules as described herein and the third therapeutic agent or procedure are administered/performed simultaneously or concurrently. For example, the delivery of one treatment is still occurring when the delivery of the third commences, e.g., there is an overlap in administration of the treatments. In other embodiments, the first agent and second agent as described herein and the third therapeutic agent or procedure are administered/performed sequentially. For example, the delivery of one treatment ceases before the delivery of the other treatment begins.

In some embodiments, combination therapy can lead to more effective treatment than monotherapy with either agent alone. In some embodiments, the combination of the first, second, and second agent is more effective (e.g., leads to a greater reduction in symptoms and/or cancer cells) than the first, second, or third treatment alone. In some embodiments, the combination therapy permits use of a lower dose of the first, the second, or the third treatment compared to the dose of the first, the second, or the third treatment normally required to achieve similar effects when administered as a monotherapy. In some embodiments, the combination therapy has a partially additive effect, wholly additive effect, or greater than additive effect.

In some embodiments, the TCRβV antibody, multispecific or multifunctional molecule is administered in combination with a therapy, e.g., a cancer therapy (e.g., one or more of cancer agents, immunotherapy, photodynamic therapy (PDT), surgery and/or radiation). The terms “chemotherapeutic,” “chemotherapeutic agent,” and “cancer agent” are used interchangeably herein. The administration of the multispecific or multifunctional molecule and the therapy, e.g., the cancer therapy, can be sequential (with or without overlap) or simultaneous. Administration of the TCRβV antibody, multispecific or multifunctional molecule can be continuous or intermittent during the course of therapy (e.g., cancer therapy). Certain therapies described herein can be used to treat cancers and non-cancerous diseases. For example, PDT efficacy can be enhanced in cancerous and non-cancerous conditions (e.g., tuberculosis) using the methods and compositions described herein (reviewed in, e.g., Agostinis, P. et al. (2011) CA Cancer J. Clin. 61:250-281).

Methods described herein include treating a cancer in a subject by using the multispecific or multifunctional molecules as described herein, e.g., using a pharmaceutical composition as described herein. Also provided are methods for reducing or ameliorating a symptom of a cancer in a subject, as well as methods for inhibiting the growth of a cancer and/or killing one or more cancer cells. In some embodiments, the methods described herein decrease the size of a tumor and/or decrease the number of cancer cells in a subject administered with a described herein or a pharmaceutical composition described herein.

In some embodiments, the cancer is a hematological cancer. In some embodiments, the hematological cancer is a leukemia or a lymphoma. As used herein, a “hematologic cancer” refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myclomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

In some embodiments, the cancer is a myeloproliferative neoplasm. e.g., primary or idiopathic myelofibrosis (MF), essential thrombocytosis (ET), polycythemia vera (PV), or chronic myelogenous leukemia (CML). In some embodiments, the cancer is myelofibrosis. In some embodiments, the subject has myelofibrosis. In some embodiments, the subject has a calreticulin mutation, e.g., a calreticulin mutation as described herein. In some embodiments, the subject does not have the JAK2-V617F mutation. In some embodiments, the subject has the JAK2-V617F mutation. In some embodiments, the subject has a MPL mutation. In some embodiments, the subject does not have a MPL mutation.

In some embodiments, the cancer is a solid cancer. Exemplary solid cancers include, but are not limited to, ovarian cancer, rectal cancer, stomach cancer, testicular cancer, cancer of the anal region, uterine cancer, colon cancer, rectal cancer, renal-cell carcinoma, liver cancer, non-small cell carcinoma of the lung, cancer of the small intestine, cancer of the esophagus, melanoma, Kaposi's sarcoma, cancer of the endocrine system, cancer of the thyroid gland, cancer of the parathyroid gland, cancer of the adrenal gland, bone cancer, pancreatic cancer, skin cancer, cancer of the head or neck, cutaneous or intraocular malignant melanoma, uterine cancer, brain stem glioma, pituitary adenoma, epidermoid cancer, carcinoma of the cervix squamous cell cancer, carcinoma of the fallopian tubes, carcinoma of the endometrium, carcinoma of the vagina, sarcoma of soft tissue, cancer of the urethra, carcinoma of the vulva, cancer of the penis, cancer of the bladder, cancer of the kidney or ureter, carcinoma of the renal pelvis, spinal axis tumor, neoplasm of the central nervous system (CNS), primary CNS lymphoma, tumor angiogenesis, metastatic lesions of said cancers, or combinations thereof.

In some embodiments, the cancer is acute lymphoblastic leukemia, acute lymphocytic leukemia, acute myelogenous leukemia, aplastic anemia, chronic myelogenous leukemia, desmoplastic small round cell tumor, Ewing's sarcoma. Hodgkin's disease, multiple myeloma, myelodysplasia, Non-Hodgkin's lymphoma, paroxysmal nocturnal hemoglobinuria, radiation poisoning, chronic lymphocytic leukemia, AL amyloidosis, essential thrombocytosis, polycythemia vera, severe aplastic anemia, neuroblastoma, breast tumors, ovarian tumors, renal cell carcinoma, autoimmune disorders, such as systemic sclerosis, osteopetrosis, inherited metabolic disorders, juvenile chronic arthritis, adrenoleukodystrophy, amegakaryocytic thrombocytopenia, sickle cell disease, severe congenital immunodeficiency. Griscelli syndrome type II, Hurler syndrome, Kostmann syndrome, Krabbe disease, metachromatic leukodystrophy, thalassemia, hemophagocytic lymphohistiocytosis, and Wiskott-Aldrich syndrome, leukemias, lymphomas, melanomas, neuroendocrine tumors, carcinomas and sarcomas. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer. In some embodiments, the cancer is a solid tumor. In some embodiments, the cancer is hematological.

In some embodiments, the combination therapy as described herein (or pharmaceutical composition as described herein) are administered in a manner appropriate to the disease to be treated or prevented. The quantity and frequency of administration will be determined by such factors as the condition of the patient, and the type and severity of the patient's disease. Appropriate dosages may be determined by clinical trials. For example, when “an effective amount” or “a therapeutic amount” is indicated, the precise amount of the pharmaceutical composition (or multispecific or multifunctional molecules) to be administered can be determined by a physician with consideration of individual differences in tumor size, extent of infection or metastasis, age, weight, and condition of the subject. In some embodiments, the pharmaceutical composition described herein can be administered at a dosage of 10⁴ to 10⁹ cells/kg body weight, e.g., 10⁵ to 10⁶ cells/kg body weight, including all integer values within those ranges. In some embodiments, the pharmaceutical composition described herein can be administered multiple times at these dosages. In some embodiments, the pharmaceutical composition described herein can be administered using infusion techniques described in immunotherapy (see, e.g., Rosenberg et al., New Eng. J. of Med. 319:1676, 1988).

In some embodiments, the combination therapy as described herein or the pharmaceutical composition as described herein is administered to the subject parentally. In some embodiments, the cells are administered to the subject intravenously, subcutaneously, intratumorally, intranodally, intramuscularly, intradermally, or intraperitoneally. In some embodiments, the cells are administered, e.g., injected, directly into a tumor or lymph node. In some embodiments, the cells are administered as an infusion (e.g., as described in Rosenberg et al., New Eng. J. of Med. 319:1676, 1988) or an intravenous push. In some embodiments, the cells are administered as an injectable depot formulation.

In some embodiments, the subject is a mammal. In some embodiments, the subject is a human, monkey, pig, dog, cat, cow, sheep, goat, rabbit, rat, or mouse. In some embodiments, the subject is a human. In some embodiments, the subject is a pediatric subject, e.g., less than 18 years of age, e.g., less than 17, 16, 15, 14, 13, 12, 11, 10, 9, 8, 7, 6, 5, 4, 3, 2, 1 or less years of age. In some embodiments, the subject is an adult, e.g., at least 18 years of age, e.g., at least 19, 20, 21, 22, 23, 24, 25, 25-30, 30-35, 35-40, 40-50, 50-60, 60-70, 70-80, or 80-90 years of age.

### Anti-Cancer Therapies

In other embodiments, the combination therapy as described herein is administered in combination with a low or small molecular weight chemotherapeutic agent. Exemplary low or small molecular weight chemotherapeutic agents include, but not limited to, 13-cis-retinoic acid (isotretinoin, ACCUTANE®), 2-CdA (2-chlorodeoxyadenosine, cladribine, LEUSTATIN™), 5-azacitidine (azacitidine, VIDAZA-®), 5-fluorouracil (5-FU, fluorouracil, ADRUCIL®), 6-mercaptopurine (6-MP, mercaptopurine, PURINETHOL®), 6-TG (6-thioguanine, thioguanine, THIOGUANINE TABLOID®), abraxane (paclitaxel protein-bound), actinomycin-D (dactinomycin, COSMEGEN®), alitretinoin (PANRETIN®), all-transretinoic acid (ATRA, tretinoin, VESANOID®), altretamine (hexamethylmelamine, HMM, HEXALEN®), amethopterin (methotrexate, methotrexate sodium, MTX, TREXALL™, RHEUMATREX®), amifostine (ETHYOL®), arabinosylcytosine (Ara-C, cytarabine, CYTOSAR-U®), arsenic trioxide (TRISENOX®), asparaginase (Erwinia L-asparaginase, L-asparaginase, ELSPAR®, KIDROLASE®), BCNU (carmustine, BiCNU®), bendamustine (TREANDA®), bexarotene (TARGRETIN®), bleomycin (BLENOXANE®), busulfan (BUSULFEX®, MYLERAN®), calcium leucovorin (Citrovorum Factor, folinic acid, leucovorin), camptothecin-11 (CPT-11, irinotecan, CAMPTOSAR®), capecitabine (XELODA®), carboplatin (PARAPLATIN®), carmustine wafer (prolifeprospan 20 with carmustine implant, GLIADEL® wafer), CCI-779 (temsirolimus, TORISEL®), CCNU (lomustine, CeeNU), CDDP (cisplatin, PLATINOL®, PLATINOL-AQ®), chlorambucil (leukeran), cyclophosphamide (CYTOXAN®, NEOSAR®), dacarbazine (DIC, DTIC, imidazole carboxamide, DTIC-DOME®), daunomycin (daunorubicin, daunorubicin hydrochloride, rubidomycin hydrochloride, CERUBIDINE®), decitabine (DACOGEN®), dexrazoxane (ZINECARD®), DHAD (mitoxantrone, NOVANTRONE®), docetaxel (TAXOTERE®), doxorubicin (ADRIAMYCIN®, RUBEX®), epirubicin (ELLENCE™), estramustine (EMCYT®), etoposide (VP-16, etoposide phosphate, TOPOSAR®, VEPESID®, ETOPOPHOS®), floxuridine (FUDR®), fludarabine (FLUDARA®), fluorouracil (cream) (CARAC™, EFUDEX®, FLUOROPLEX®), gemcitabine (GEMZAR®), hydroxyurea (HYDREA®, DROXIA™, MYLOCEL™), idarubicin (IDAMYCIN®), ifosfamide (IFEX®), improsulfan, ixabepilone (IXEMPRA™), LCR (leurocristine, vincristine, VCR, ONCOVIN®, VINCASAR PFS®), L-PAM (L-sarcolysin, melphalan, phenylalanine mustard, ALKERAN®), mechlorethamine (mechlorethamine hydrochloride, mustine, nitrogen mustard, MUSTARGEN®), mesna (MESNEX™), mitomycin (mitomvcin-C, MTC, MUTAMYCIN®), nelarabine (ARRANON®), oxaliplatin (ELOXATIN™), paclitaxel (TAXOL, ONXAL™), pegaspargase (PEG-L-asparaginase, ONCOSPAR®), PEMETREXED (ALIMTA®), pentostatin (NIPENT®), piposulfan, procarbazine (MATULANE®), streptozocin (ZANOSAR®), temozolomide (TEMODAR®), teniposide (VM-26, VUMON®), TESPA (thiophosphoamide, thiotepa, TSPA, THIOPLEX®), topotecan (HYCAMTINX), vinblastine (vinblastine sulfate, vincaleukoblastine, VLB, ALKABAN-AQ-®, VELBAN®), vinorelbine (vinorelbine tartrate, NAVELBINE®), and vorinostat (ZOLINZA®).

In another embodiment, the combination therapy as described herein is administered in conjunction with a biologic. Biologics useful in the treatment of cancers are known in the art and a binding molecule as described herein may be administered, for example, in conjunction with such known biologics. For example, the FDA has approved the following biologics for the treatment of breast cancer: HERCEPTIN® (trastuzumab, Genentech Inc., South San Francisco, Calif.; a humanized monoclonal antibody that has anti-tumor activity in HER2-positive breast cancer); FASLODEX® (fulvestrant, AstraZeneca Pharmaceuticals. LP, Wilmington, Del.: an estrogen-receptor antagonist used to treat breast cancer); ARIMIDEX® (anastrozole, AstraZeneca Pharmaceuticals, LP; a nonsteroidal aromatase inhibitor which blocks aromatase, an enzyme needed to make estrogen); Aromasinm® (exemestane, Pfizer Inc., New York, N.Y.; an irreversible, steroidal aromatase inactivator used in the treatment of breast cancer); FEMARA® (letrozole, Novartis Pharmaceuticals. East Hanover, N.J.; a nonsteroidal aromatase inhibitor approved by the FDA to treat breast cancer); and NOLVADEX® (tamoxifen, AstraZeneca Pharmaceuticals, LP; a nonsteroidal antiestrogen approved by the FDA to treat breast cancer). Other biologics with which the binding molecules as described herein may be combined include: AVASTIN® (bevacizumab, Genentech Inc.; the first FDA-approved therapy designed to inhibit angiogenesis); and ZEVALIN® (ibritumomab tiuxetan, Biogen Idec, Cambridge, Mass.; a radiolabeled monoclonal antibody currently approved for the treatment of B-cell lymphomas).

In addition, the FDA has approved the following biologics for the treatment of colorectal cancer: AVASTIN®, ERBITUX® (cetuximab, ImClone Systems Inc., New York, N.Y., and Bristol-Myers Squibb, New York, N.Y.; is a monoclonal antibody directed against the epidermal growth factor receptor (EGFR)); GLEEVEC® (imatinib mesylate; a protein kinase inhibitor); and ERGAMISOL® (levamisole hydrochloride, Janssen Pharmaceutica Products, LP, Titusville, N.J.; an immunomodulator approved by the FDA in 1990 as an adjuvant treatment in combination with 5-fluorouracil after surgical resection in patients with Dukes' Stage C colon cancer).

For the treatment of lung cancer, exemplary biologics include TARCEVA® (erlotinib HCL, OSI Pharmaceuticals Inc., Melville, N.Y.; a small molecule designed to target the human epidermal growth factor receptor 1 (HER1) pathway).

For the treatment of multiple myeloma, exemplary biologics include VELCADE® (bortezomib, Millennium Pharmaceuticals, Cambridge Mass., a proteasome inhibitor). Additional biologics include THALIDOMID® (thalidomide, Clegene Corporation. Warren, N.J.; an immunomodulatory agent and appears to have multiple actions, including the ability to inhibit the growth and survival of myeloma cells and anti-angiogenesis).

Additional exemplary cancer therapeutic antibodies include, but are not limited to, 3F8, abagovomab, adecatumumab, afutuzumab, alacizumab pegol, alemtuzumab (CAMPATH®, MABCAMPATH®), altumomab pentetate (HYBRI-CEAKER®), anatumomab mafenatox, anrukinzumab (IMA-638), apolizumab, arcitumomab (CEA-SCAN®), bavituximab, bectumomab (LYMPHOSCAN®), belimumab (BENLYSTA®, LYMPHOSTAT-B®), besilesomab (SCINTIMUN®), bevacizumab (AVASTIN®), bivatuzumab mertansine, blinatumomab, brentuximab vedotin, cantuzumab mertansine, capromab pendetide (PROSTASCINT®), catumaxomab (REMOVAB®), CC49, cetuximab (C225, ERBITUX®), citatuzumab bogatox, cixutumumab, clivatuzumab tetraxetan, conatumumab, dacetuzumab, denosumab (PROLIA-), detumomab, ecromeximab, edrecolomab (PANOREX®), elotuzumab, epitumomab cituxetan, epratuzumab, ertumaxomab (REXOMUN®), etaracizumab, farletuzumab, figitumumab, fresolimumab, galiximab, gemtuzumab ozogamicin (MYLOTARG®), girentuximab, glembatumumab vedotin, ibritumomab (ibritumomab tiuxetan, ZEVALIN®), igovomab (INDIMACIS-125®), intetumumab, inotuzumab ozogamicin, ipilimumab, iratumumab, labetuzumab (CEA-CIDE®), lexatumumab, lintuzumab, lucatumumab, lumiliximab, mapatumumab, matuzumab, milatuzumab, minretumomab, mitumomab, nacolomab tafenatox, naptumomab estafenatox, necitumumab, nimotuzumab (THERACIM®, THERALOC®), nofetumomab merpentan (VERLUMA®), of atumumab (ARZERRA®), olaratumab, oportuzumab monatox, oregovomab (OVAREX®), panitumumab (VECTIBIX®), pemtumomab (THERAGYN®), pertuzumab (OMNITARG®), pintumomab, pritumumab, ramucirumab, ranibizumab (LUCENTIS®), rilotumumab, rituximab (MABTHERA®, RITUXAN®), robatumumab, satumomab pendetide, sibrotuzumab, siltuximab, sontuzumab, tacatuzumab tetraxetan (AFP-CIDE®), taplitumomab paptox, tenatumomab, TGN1412, ticilimumab (tremelimumab), tigatuzumab, TNX-650, tositumomab (BEXXAR®), trastuzumab (HERCEPTIN®), tremelimumab, tucotuzumab celmoleukin, veltuzumab, volociximab, votumumab (HUMASPECT®), zalutumumab (HUMAX-EGFR®), and zanolimumab (HUMAX-CD4®).

Additional exemplary cancer therapeutic agents include, but are not limited to alkylating agents (e.g., thiotepa and cyclosphosphamide), alkyl sulfonates (e.g., busulfan, improsulfan, and piposulfan), aziridines (e.g., such as benzodopa, carboquone, meturedopa, and uredopa), ethylenimines and methylamelamines (e.g., altretamine, triethylencmclamine, trietylcnephosphoramide, triethiylenethiophosphoramide, and trimethylolomelamine), acetogenins (e.g., bullatacin and bullatacinone), camptothecin (e.g., topotecan); bryostatin; callystatin, CC-1065 (e.g., adozelesin, carzelesin and bizelesin), cryptophycins (e.g., cryptophycin 1 and cryptophycin 8), dolastatin, duocarmycin (e.g., KW-2189 and CB1-TM1); eleutherobin, pancratistatin, sarcodictyin, spongistatin, nitrogen mustards (e.g., chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard) nitrosureas (e.g., carmustine, chlorozotocin, fotemustine, lomustine, nimustine, and ranimnustine), aclacinomysins, actinomycin, authramycin, azaserine, bleomycins, cactinomycin, carabicin, canninomycin, carzinophilin, chromomycinis, dactinomycin, daunorubicin, detorubicin, 6-diazo-5-oxo-L-norleucine, doxorubicin (e.g., morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin, anti-metabolites (e.g., methotrexate and 5-fluorouracil (5-FU)), folic acid analogues (e.g., denopterin, pteropterin, and trimetrexate), purine analogs (e.g., fludarabine, 6-mercaptopurine, thiamiprine, and thioguanine) pyrimidine analogs (e.g., ancitabine, azacitidine, 6-azauridine, carmofur, cytarabine, dideoxyuridine, doxifluridine, enocitabine, and floxuridine); androgens (e.g., calusterone, dromostanolone propionate, epitiostanol, mepitiostane, and testolactone), anti-adrenals (e.g., as mitotane and trilostane), folic acid replenisher (e.g., frolinic acid; aceglatone; aldophosphamide glycoside), aminolevulinic acid, eniluracil, amsacrine, bestrabucil, bisantrene, edatrexate, defofamine, demecolcine, diaziquone, elformithine, elliptinium acetate, epothilone, etoglucid, gallium nitrate, hydroxyurea, lentinan, lonidainine, maytansinoids (e.g., such as maytansine and ansamitocins), mitoguazone, mitoxantrone, mopidanmol, nitraerine, pentostatin, phenamet, pirarubicin, losoxantrone, podophyllinic acid, 2-ethylhydrazide, procarbazine, PSKpolysaccharide complex, razoxane, rhizoxin, sizofiran, spirogermanium, tenuazonic acid, triaziquone, 2,2′,2″-trichlorotriethylamine, trichothecenes (e.g., T-2 toxin, verracurin A, roridin A and anguidine), urethan, vindesine, dacarbazine, mannomustine, mitobronitol, mitolactol, pipobroman, gacytosine, arabinoside (e.g., Ara-C), cyclophosphamide; toxoids (e.g., paclitaxel and docetaxel), gemcitabine, 6-thioguanine, mercaptopurine, platinum coordination complexes (cisplatin, oxaliplatin, and carboplatin), vinblastine, platinum, etoposide (e.g., VP-16), ifosfamide, mitoxantrone, vincristine, vinorelbine, novantrone, teniposide, edatrexate, daunomycin, aminopterin, xeloda, ibandronate, irinotecan (e.g., CPT-11), topoisomerase inhibitor, difluorometlhylomithine (DMFO), retinoids (retinoic acid), capecitabine, carboplatin, procarbazine, and plicamycin.

In some embodiments, the combination therapy as described herein are administered in combination with a viral cancer therapeutic agent. Exemplary viral cancer therapeutic agents include, but not limited to, vaccinia virus (vvDD-CDSR), carcinoembryonic antigen-expressing measles virus, recombinant vaccinia virus (TK-deletion plus GM-CSF), Seneca Valley virus-001, Newcastle virus, coxsackie virus A21, GL-ONC1, EBNA1 C-terminal/LMP2 chimeric protein-expressing recombinant modified vaccinia Ankara vaccine, carcinoembryonic antigen-expressing measles virus, G207 oncolytic virus, modified vaccinia virus Ankara vaccine expressing p53, OncoVEX GM-CSF modified herpes-simplex 1 virus, fowlpox virus vaccine vector, recombinant vaccinia prostate-specific antigen vaccine, human papillomavirus 16/18 L1 virus-like particle/AS04 vaccine, MVA-EBNA1/LMP2 Inj. vaccine, quadrivalent HPV vaccine, quadrivalent human papillomavirus (types 6, 11, 16, 18) recombinant vaccine (GARDASIL®), recombinant fowlpox-CEA(6D)/TRICOM vaccine, recombinant vaccinia-CEA(6D)-TRICOM vaccine, recombinant modified vaccinia Ankara-5T4 vaccine, recombinant fowlpox-TRICOM vaccine, oncolytic herpes virus NV1020, HPV L1 VLP vaccine V504, human papillomavirus bivalent (types 16 and 18) vaccine (CERVARIX®), herpes simplex virus HF10, Ad5CMV-p53 gene, recombinant vaccinia DF3/MUC1 vaccine, recombinant vaccinia-MUC-1 vaccine, recombinant vaccinia-TRICOM vaccine, ALVAC MART-1 vaccine, replication-defective herpes simplex virus type I (HSV-1) vector expressing human Preproenkephalin (NP2), wild-type reovirus, reovirus type 3 Dearing (REOLYSIN®), oncolytic virus HSV1716, recombinant modified vaccinia Ankara (MVA)-based vaccine encoding Epstein-Barr virus target antigens, recombinant fowlpox-prostate specific antigen vaccine, recombinant vaccinia prostate-specific antigen vaccine, recombinant vaccinia-B7.1 vaccine, rAd-p53 gene, Ad5-delta24RGD, HPV vaccine 580299, JX-594 (thymidine kinase-deleted vaccinia virus plus GM-CSF), HPV-16/18 L1/AS04, fowlpox virus vaccine vector, vaccinia-tyrosinase vaccine, MEDI-517 HPV-16/18 VLP AS04 vaccine, adenoviral vector containing the thymidine kinase of herpes simplex virus TK99UN, HspE7, FP253/Fludarabine, ALVAC(2) melanoma multi-antigen therapeutic vaccine, ALVAC-hB7.1, canarypox-hIL-12 melanoma vaccine, Ad-REIC/Dkk-3, rAd-IFN SCH 721015, TIL-Ad-INFg, Ad-ISF35, and coxsackievirus A21 (CVA21, CAVATAK®).

In some embodiments, the combination therapy as described herein are administered in combination with a nanopharmaceutical. Exemplary cancer nanopharmaceuticals include, but not limited to, ABRAXANE® (paclitaxel bound albumin nanoparticles), CRLX101 (CPT conjugated to a linear cyclodextrin-based polymer). CRLX288 (conjugating docetaxel to the biodegradable polymer poly (lactic-co-glycolic acid)), cytarabine liposomal (liposomal Ara-C, DEPOCYT™), daunorubicin liposomal (DAUNOXOME®), doxorubicin liposomal (DOXIL®, CAELYX®), encapsulated-daunorubicin citrate liposome (DAUNOXOME®), and PEG anti-VEGF aptamer (MACUGEN®).

In some embodiments, the combination therapy as described herein are administered in combination with paclitaxel or a paclitaxel formulation, e.g., TAXOL®, protein-bound paclitaxel (e.g., ABRAXANE®). Exemplary paclitaxel formulations include, but are not limited to, nanoparticle albumin-bound paclitaxel (ABRAXANE®, marketed by Abraxis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., 2′-paclitaxel methyl 2-glucopyranosyl succinate, see Liu et al., Bioorganic & Medicinal Chemistry Letters (2007) 17:617-620).

Exemplary RNAi and antisense RNA agents for treating cancer include, but not limited to, CALAA-01, siG12D LODER (Local Drug EluteR), and ALN-VSP02.

Other cancer therapeutic agents include, but not limited to, cytokines (e.g., aldesleukin (IL-2, Interleukin-2, PROLEUKIN®), alpha Interferon (IFN-alpha, Interferon alfa, INTRON® A (Interferon alfa-2b), ROFERON-A® (Interferon alfa-2a)), Epoetin alfa (PROCRIT®), filgrastim (G-CSF, Granulocyte-Colony Stimulating Factor, NEUPOGEN®), GM-CSF (Granulocyte Macrophage Colony Stimulating Factor, sargramostim, LEUKINE™), IL-11 (Interleukin-11, oprelvekin, NEUMEGA®), Interferon alfa-2b (PEG conjugate) (PEG interferon, PEG-INTRON™), and pegfilgrastim (NEULASTA™)), hormone therapy agents (e.g., aminoglutethimide (CYTADREN®), anastrozole (ARIMIDEX®), bicalutamide (CASODEX®), exemestane (AROMASIN®), fluoxymesterone (HALOTESTIN®), flutamide (EULEXIN®), fulvestrant (FASLODEX®), goserelin (ZOLADEX®), letrozole (FEMARA®), leuprolide (ELIGARD™, LUPRON®, LUPRON DEPOT®, VIADUR™), megestrol (megestrol acetate, MEGACE®), nilutamide (ANANDRON®, NILANDRON®), octreotide (octreotide acetate, SANDOSTATIN®, SANDOSTATIN LAR®), raloxifene (EVISTA®), romiplostim (NPLATE®), tamoxifen (NOVALDEX®), and toremifene (FARESTON®)), phospholipasc A2 inhibitors (e.g., anagrelide (AGRYLIN®)), biologic response modifiers (e.g., BCG (THERACYS®, TICE®), and Darbepoetin alfa (ARANESPT®)), target therapy agents (e.g., bortezomib (VELCADE®), dasatinib (SPRYCEL™), denileukin diftitox (ONTAK®), erlotinib (TARCEVA®), everolimus (AFINITOR®), gefitinib (IRESSA®), imatinib mesylate (ST1-571, GLEEVEC™), lapatinib (TYKERB®), sorafenib (NEXAVAR®), and SU11248 (sunitinib, SUTENT®)), immunomodulatory and antiangiogenic agents (e.g., CC-5013 (lenalidomide, REVLIMID®), and thalidomide (THALOMID®)), glucocorticosteroids (e.g., cortisone (hydrocortisone, hydrocortisone sodium phosphate, hydrocortisone sodium succinate, ALA-CORT®, HYDROCORT ACETATE®, hydrocortone phosphate LANACORT®, SOLU-CORTEF®), decadron (dexamethasone, dexamethasone acetate, dexamethasone sodium phosphate, DEXASONE®, DIODEX®, HEXADROL®, MAXIDEX®), methylprednisolone (6-methylprednisolone, methylprednisolone acetate, methylprednisolone sodium succinate, DURALONE®, MEDRALONE®, MEDROL®, M-PREDNISOL®, SOLU-MEDROL®), prednisolone (DELTA-CORTEF®, ORAPRED®, PEDIAPRED®, PRELONE®), and prednisone (DELTASONE®, LIQUID PRED®, METICORTEN®, ORASONE®)), and bisphosphonates (e.g., pamidronate (AREDIA®), and zoledronic acid (ZOMETA®)).

In some embodiments, the combination therapy as described herein are used in combination with a tyrosine kinase inhibitor (e.g., a receptor tyrosine kinase (RTK) inhibitor). Exemplary tyrosine kinase inhibitor include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., an antibody against VEGF, a VEGF trap, a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-β inhibitor)), a RAF-1 inhibitor, a KIT inhibitor and a RET inhibitor. In some embodiments, the anti-cancer agent used in combination with the AHCM agent is selected from the group consisting of: axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU1248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAMPATH®), gemtuzumab ozogamicin (MYLOTARG®), ENMD-2076, PCI-32765, AC220, dovitinib lactate (TK1258, CH1R-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, XL228, AEE788, AG-490, AST-6, BMS-599626, CUDC-101, PD153035, pelitinib (EKB-569), vandetanib (zactima), WZ3146, WZ4002, WZ8040, ABT-869 (linifanib), AEE788, AP24534 (ponatinib), AV-951(tivozanib), axitinib, BAY 73-4506 (regorafenib), brivanib alaninate (BMS-582664), brivanib (BMS-540215), cediranib (AZD2171), CH1R-258 (dovitinib), CP 673451, CYC116, E7080, Ki8751, masitinib (AB1010), MGCD-265, motesanib diphosphate (AMG-706), MP-470, OS1-930, Pazopanib Hydrochloride. PD173074, Sorafenib Tosylate (Bay 43-9006), SU 5402, TSU-68(SU6668), vatalanib, XL880 (GSK1363089, EXEL-2880). Selected tyrosine kinase inhibitors are chosen from sunitinib, erlotinib, gefitinib, or sorafenib. In some embodiments, the tyrosine kinase inhibitor is sunitinib.

In some embodiments, the combination therapy as described herein are administered in combination with one of more of: an anti-angiogenic agent, or a vascular targeting agent or a vascular disrupting agent. Exemplary anti-angiogenic agents include, but are not limited to, VEGF inhibitors (e.g., anti-VEGF antibodies (e.g., bevacizumab); VEGF receptor inhibitors (e.g., itraconazole); inhibitors of cell proliferatin and/or migration of endothelial cells (e.g., carboxyamidotriazole, TNP-470); inhibitors of angiogenesis stimulators (e.g., suramin), among others. A vascular-targeting agent (VTA) or vascular disrupting agent (VDA) is designed to damage the vasculature (blood vessels) of cancer tumors causing central necrosis (reviewed in, e.g., Thorpe, P. E. (2004) Clin. Cancer Res. Vol. 10:415-427). VTAs can be small-molecule. Exemplary small-molecule VTAs include, but are not limited to, microtubule destabilizing drugs (e.g., combretastatin A-4 disodium phosphate (CA4P), ZD6126, AVE8062, Oxi 4503); and vadimezan (ASA404).

### Immune Checkpoint Inhibitors

In other embodiments, methods described herein comprise use of an immune checkpoint inhibitor in combination with the combination therapy as described herein. The methods can be used in a therapeutic protocol in vivo.

In some embodiments, an immune checkpoint inhibitor inhibits a checkpoint molecule. Exemplary checkpoint molecules include but are not limited to CTLA4, PD1, PD-L1, PD-L2, TIM3, LAG3, CD160, 2B4, CD80, CD86, B7-H3 (CD276), B7-H4 (VTCN1), HVEM (TNFRSF14 or CD270), BTLA, KIR, MHC class I, MHC class II, GAL9, VISTA, BTLA, TIGIT, LAIR1, and A2aR. See, e.g., Pardoll. Nat. Rev. Cancer 12.4(2012):252-64, incorporated herein by reference.

In some embodiments, the immune checkpoint inhibitor is a PD-1 inhibitor, e.g., an anti-PD-1 antibody such as Nivolumab, Pembrolizumab or Pidilizumab. Nivolumab (also called MDX-1106, MDX-1106-04, ONO-4538, or BMS-936558) is a fully human IgG4 monoclonal antibody that specifically inhibits PD1. See, e.g., U.S. Pat. No. 8,008,449 and WO2006/121168. Pembrolizumab (also called Lambrolizumab, MK-3475, MK03475, SCH-900475 or KEYTRUDA®; Merck) is a humanized IgG4 monoclonal antibody that binds to PD-1. See, e.g., Hamid, O. et al. (2013) New England Journal of Medicine 369 (2): 134-44, U.S. Pat. No. 8,354,509 and WO2009/114335. Pidilizumab (also called CT-011 or Cure Tech) is a humanized IgG1 k monoclonal antibody that binds to PD1. See, e.g., WO2009/101611. In some embodiments, the inhibitor of PD-1 is an antibody molecule having a sequence substantially identical or similar thereto, e.g., a sequence at least 85%, 90%, 95% identical or higher to the sequence of Nivolumab, Pembrolizumab or Pidilizumab. Additional anti-PD1 antibodies, e.g., AMP 514 (Amplimmune), are described, e.g., in U.S. Pat. No. 8,609,089, US 2010028330, and/or US 20120114649.

In some embodiments, the PD-1 inhibitor is an immunoadhesin, e.g., an immunoadhesin comprising an extracellular/PD-1 binding portion of a PD-1 ligand (e.g., PD-L1 or PD-L2) that is fused to a constant region (e.g., an Fc region of an immunoglobulin). In some embodiments, the PD-1 inhibitor is AMP-224 (B7-DCIg, e.g., described in WO2011/066342 and WO2010/027827), a PD-L2 Fc fusion soluble receptor that blocks the interaction between B7-H1 and PD-1.

In some embodiments, the immune checkpoint inhibitor is a PD-L1 inhibitor, e.g., an antibody molecule. In some embodiments, the PD-L1 inhibitor is YW243.55.S70, MPDL3280A, MEDI-4736, MSB-0010718C, or MDX-1105. In some embodiments, the anti-PD-L1 antibody is MSB0010718C (also called A09-246-2; Merck Serono), which is a monoclonal antibody that binds to PD-L1, Exemplary humanized anti-PD-L1 antibodies are described, e.g., in WO2013/079174. In some embodiments, the PD-L1 inhibitor is an anti-PD-L1 antibody, e.g., YW243.55.S70. The YW243.55.S70 antibody is described, e.g., in WO 2010/077634. In some embodiments, the PD-L1 inhibitor is MDX-1105 (also called BMS-936559), which is described, e.g., in WO2007/005874. In some embodiments, the PD-L1 inhibitor is MDPL3280A (Genentech/Roche), which is a human Fc-optimized IgG1 monoclonal antibody against PD-L1. See, e.g., U.S. Pat. No. 7,943,743 and U.S. Publication No.: 20120039906. In some embodiments, the inhibitor of PD-L1 is an antibody molecule having a sequence substantially identical or similar thereto, e.g., a sequence at least 85%, 90%, 95% identical or higher to the sequence of YW243.55.S70, MPDL3280A, MEDI-4736, MSB-0010718C, or MDX-1105.

In some embodiments, the immune checkpoint inhibitor is a PD-L2 inhibitor, e.g., AMP-224 (which is a PD-L2 Fc fusion soluble receptor that blocks the interaction between PD1 and B7-H1. See, e.g., WO2010/027827 and WO2011/066342.

In some embodiments, the immune checkpoint inhibitor is a LAG-3 inhibitor, e.g., an anti LAG-3 antibody molecule. In some embodiments, the anti-LAG-3 antibody is BMS-986016 (also called BMS986016; Bristol-Myers Squibb). BMS-986016 and other humanized anti-LAG-3 antibodies are described, e.g., in US 2011/0150892, WO2010/019570, and WO2014/008218.

In some embodiments, the immune checkpoint inhibitor is a TIM-3 inhibitor, e.g., anti-TIM3 antibody molecule, e.g., described in U.S. Pat. No. 8,552,156, WO 2011/155607, EP 2581113 and U.S. Publication No.: 2014/044728.

In some embodiments, the immune checkpoint inhibitor is a CTLA-4 inhibitor, e.g., anti-CTLA-4 antibody molecule. Exemplary anti-CTLA4 antibodies include Tremelimumab (IgG2 monoclonal antibody from Pfizer, formerly known as ticilimumab, CP-675,206); and Ipilimumab (also called MDX-010, CAS No. 477202-00-9). Other exemplary anti-CTLA-4 antibodies are described, e.g., in U.S. Pat. No. 5,811,097.

### Method of Expanding Cells

Any of the compositions and methods described herein can be used to expand an immune cell population. An immune cell provided herein includes an immune cell derived from a hematopoictic stem cell or an immune cell derived from a non-hematopoietic stem cell, e.g., by differentiation or de-differentiation.

An immune cell includes a hematopoietic stem cell, progeny thereof and/or cells that have differentiated from said HSC, e.g., lymphoid cells or myeloid cells. An immune cell can be an adaptive immune cell or an innate immune cell. Examples of immune cells include T cells, B cells, Natural Killer cells, Natural Killer T cells, neutrophils, dendritic cells, monocytes, macrophages, and granulocytes.

In some embodiments, an immune cell is a T cell. In some embodiments, a T cell includes a CD4+ T cell, a CD8+ T cell, a TCR alpha-beta T cell, a TCR gamma-delta T cell. In some embodiments, a T cell comprises a memory T cell (e.g., a central memory T cell, or an effector memory T cell (e.g., a TEMRA) or an effector T cell. In some embodiments, a T cell comprises a tumor infiltrating lymphocyte (TIL).

In some embodiments, an immune cell is an NK cell.

In some embodiments, an immune cell is a TIL. TILs are immune cells (e.g., T cells, B cells or NK cells) that can be found in a tumor or around a tumor (e.g., in the stroma or tumor microenvironment of a tumor), e.g., a solid tumor, e.g., as described herein. TILs can be obtained from a sample from a subject having cancer, e.g., a biopsy or a surgical sample. In some embodiments, TILs can be expanded using a method as described herein. In some embodiments, a population of expanded TILs, e.g., expanded using a method as described herein, can be administered to a subject to treat a disease, e.g., a cancer.

In some embodiments, immune cells, e.g., T cells (e.g., TILs), can be obtained from a unit of blood collected from a subject using any number of techniques known to the skilled artisan, such as Ficoll™ separation. In one aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and, optionally, to place the cells in an appropriate buffer or media for subsequent processing steps. In one embodiment, the cells are washed with phosphate buffered saline (PBS). In an alternative embodiment, the wash solution lacks calcium and may lack magnesium or may lack many if not all divalent cations. The methods described herein can include more than one selection step, e.g., more than one depletion step.

In one embodiment, the methods as described herein can utilize culture media conditions comprising DMEM, DMEM F12, RPMI 1640, and/or AIM V media. The media can be supplemented with glutamine, HEPES buffer (e.g., 10 mM), serum (e.g., heat-inactivated serum, e.g., 10%), and/or beta mercaptoethanol (e.g., 55 uM). In some embodiments, the culture conditions as described herein comprise one or more supplements, cytokines, growth factors, or hormones. In some embodiments, the culture condition comprises one or more of IL-2, IL-15, or IL-7, or a combination thereof.

Immune effector cells such as T cells may be activated and expanded generally using methods as described, for example, in U.S. Pat. Nos. 6,352,694; 6,534,055; or 6,905,680. Generally, a population of immune cells, may be expanded by contact with an agent that stimulates a CD3/TCR complex associated signal and a ligand that stimulates a costimulatory molecule on the surface of the T cells; and/or by contact with a cytokine, e.g., IL-2, IL-15 or IL-7. T cell expansion protocols can also include stimulation, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besancçon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In some embodiments, a TIL population can also be expanded by methods known in the art. For example, a population of TILs can be expanded as described in Hall et al., Journal for ImmunoTherapy of Cancer (2016) 4:61, the entire contents of which are hereby incorporated by reference. Briefly, TILs can be isolated from a sample by mechanical and/or physical digestion. The resultant TIL population can be stimulated with an anti-CD3 antibody in the presence of non-dividing feeder cells. In some embodiments, the TIL population can be cultured, e.g., expanded, in the presence of IL-2, e.g., human IL-2. In some embodiments, the TIL cells can be cultured, e.g., expanded for a period of at least 1-21 days, e.g., at least 1, 2, 3, 4, 5, 6, 7, 8, 9, 10, 11, 12, 13, 14, 15, 16, 17, 18, 19, 20 or 21 days.

As described herein, in some embodiments, an immune cell population (e.g., a T cell (e.g., a TEMRA cell or a TIL population) can be expanded by contacting the immune cell population with the multifunctional polypeptide molecule as described herein.

In some embodiments, the expansion occurs in vivo, e.g., in a subject. In some embodiments, a subject is administered the multifunctional polypeptide molecule as described herein resulting in expansion of immune cells in vivo.

In some embodiments, the expansion occurs ex vivo, e.g., in vitro. In some embodiments, cells from a subject, e.g., T cells, e.g., TIL cells, are expanded in vitro with the multifunctional polypeptide molecule as described herein. In some embodiments, the expanded TILs are administered to the subject to treat a disease or a symptom of a disease.

In some embodiments, a method of expansion as described herein results in an expansion of at least 1.1-10 fold, 10-20 fold, or 20-50 fold expansion. In some embodiments, the expansion is at least 1.1, 1.2, 1.3, 1.4, 1.5, 2, 3, 4, 5, 6, 7, 8, 9, 10, 15, 20, 25, 30, 35, 40, 45, 50, 55, 60, 65, 70, 75, 80, 85, 90, 100, 200, 300, 400, 500, 600, 700, 800, 900, 1000, 1500, 2000, 2500, 3000, 3500, 400, 4500, 5000, 5500, 6000, 6500, 7000, 7500, 8000, 8500, 9000, 9500, or 10000 fold expansion.

In some embodiments, a method of expansion as described herein comprises culturing, e.g., expanding, the cells for at least about 4 hours, 6 hours, 10 hours, 12 hours, 15 hours, 18 hours, 20 hours, or 22 hours. In some embodiments, a method of expansion as described herein comprises culturing, e.g., expanding, the cells for at least 1, 2, 3, 4, 5, 6, 7, 8, 9, 10, 11, 12, 13, 14, 15, 1,6 17, 18, 19, 20 or 21 days. In some embodiments, a method of expansion as described herein comprises culturing, e.g., expanding, the cells for at least about 1 week, 2 weeks, 3 weeks, 4 weeks, 5 weeks, 6 weeks, 7 weeks or 8 weeks.

In some embodiments, a method of expansion as described herein is performed on immune cells obtained from a healthy subject.

In some embodiments, a method of expansion as described herein is performed on immune cells (e.g., TILs) obtained from a subject having a disease, e.g., a cancer, e.g., a solid tumor as described herein.

In some embodiments, a method of expansion as described herein further comprises contacting the population of cells with an agent, that promotes, e.g., increases, immune cell expansion. In some embodiments, the agent comprises an immune checkpoint inhibitor, e.g., a PD-1 inhibitor, a LAG-3 inhibitor, a CTLA4 inhibitor, or a TIM-3 inhibitor. In some embodiments, the agent comprises a 4-1BB agonist, e.g., an anti-4-1BB antibody.

Without wishing to be bound by theory, in some embodiments, the multifunctional polypeptide molecule as described herein can expand, e.g., selectively or preferentially expand, T cells expressing a T cell receptor (TCR) comprising a TCR alpha and/or TCR beta molecule, e.g., TCR alpha-beta T cells (as T cells). In some embodiments, the multifunctional polypeptide molecule as described herein does not expand, or induce proliferation of T cells expressing a TCR comprising a TCR gamma and/or TCR delta molecule, e.g., TCR gamma-delta T cells (γδ T cells). In some embodiments, the multifunctional polypeptide molecule as described herein selectively or preferentially expands αβ T cells over γδ T cells.

Without wishing to be bound by theory, in some embodiments, γδ T cells are associated with cytokine release syndrome (CRS) and/or neurotoxicity (NT). In some embodiments, the multispecific or multifunctional molecules as described herein result in selective expansion of non-γδ T cells, e.g., expansion of as T cells, thus reducing CRS and/or NT.

In some embodiments, any of the compositions or methods as described herein result in an immune cell population having a reduction of, e.g., depletion of, γδ T cells. In some embodiments, the immune cell population is contacted with an agent that reduces, e.g., inhibits or depletes, γδ T cells, e.g., an anti-IL-17 antibody or an agent that binds to a TCR gamma and/or TCR delta molecule.

In some embodiments, the multifunctional polypeptide molecule as described herein results in expansion of an immune cell, e.g., a T cell, a tumor infiltrating lymphocyte (TIL), an NK cell, or other immune cells (e.g., as described herein).

In some embodiments, binding of the multifunctional polypeptide molecule as described herein to a TCRβV region results in one, two, three or all of: (i) reduced T cell proliferation kinetics; (ii) cell killing, e.g., target cell killing, e.g. cancer cell killing, e.g., as measured by an assay of Example 4. (iii) increased Natural Killer (NK) cell proliferation, e.g., expansion; or (iv) expansion, e.g., at least about 1.1-10 expansion (e.g., at least about 1.1, 1.2, 1.3, 1.4, 1.5, 2, 3, 4, 5, 6, 7, 8, 9, or 10 fold expansion), of a population of T cells having a memory-like phenotype, e.g., as described herein, e.g., wherein (i)-(iv) are relative to the non-TCRβV-binding T cell engager.

In some embodiments, the method further comprises contacting the population of cells with an agent that promotes, e.g., increases, immune cell expansion. In some embodiments, the agent includes an immune checkpoint inhibitor, e.g., as described herein. In some embodiments, the agent includes a 4-1BB (CD127) agonist, e.g., an anti-4-1BB antibody.

In some embodiments, the method further comprises comprising contacting the population of cells with a non-dividing population of cells, e.g., feeder cells, e.g., irradiated allogenic human PBMCs.

In some embodiments, expansion of the population of immune cells, is compared to expansion of a similar population of cells with an antibody that binds to: a CD3 molecule, e.g., CD3 epsilon (CD3e) molecule; or a TCR alpha (TCRα) molecule.

In some embodiments, expansion of the population of immune cells, is compared to expansion of a similar population of cells not contacted with the anti-TCRβV antibody molecule or the multispecific or multifunctional molecules as described herein.

In some embodiments, expansion of the population of memory effector T cells, e.g., TEM cells, e.g., TEMRA cells, is compared to expansion of a similar population of cells with an antibody that binds to: a CD3 molecule, e.g., CD3 epsilon (CD3e) molecule; or a TCR alpha (TCRα) molecule.

In some embodiments, the method results in expansion of, e.g., selective or preferential expansion of, T cells expressing a T cell receptor (TCR) comprising a TCR alpha and/or TCR beta molecule, e.g., TCR alpha-beta T cells (as T cells).

In some embodiments, the method results in expansion of αβT cells over expansion of T cells expressing a TCR comprising a TCR gamma and/or TCR delta molecule, e.g., TCR gamma-delta T cells (γδ T cells). In some embodiments, expansion of αβT cells over γδ T cells results in reduced production of cytokines associated with CRS. In some embodiments, expansion of αβ T cells over γδ T cells results in immune cells that have reduced capacity to, e.g., are less prone to, induce CRS upon administration into a subject.

In some embodiments, an immune cell population (e.g., T cells (e.g., TEMRA cells or TILs) or NK cells) cultured in the presence of, e.g., expanded with, the multifunctional polypeptide molecule as described herein does not induce CRS and/or NT when administered into a subject, e.g., a subject having a disease or condition as described herein.

In some embodiments, provided herein is a multifunctional polypeptide molecule as described herein comprising a non-murine, e.g., a human-like antibody molecule (e.g., a human or humanized antibody molecule), which binds, e.g., specifically binds, to a T cell receptor beta variable (TCRβ4V) region. In some embodiments, binding of the multifunctional polypeptide molecule as described herein results in expansion, e.g., at least about 1.1-50 fold expansion (e.g., at least about 1.5-40 fold, 2-35 fold, 3-30 fold, 5-25 fold, 8-20 fold, or 10-15 fold expansion), of a population of T cells, e.g., a population of T cells having a memory-like phenotype, e.g., CD45RA+ CCR7− T cells. In some embodiments, the population of T cells having a memory-like phenotype comprises CD4+ and/or CD8+ T cells. In some embodiments, the population of T cells having a memory-like phenotype comprises a population of memory T cells, e.g., T effector memory (TEM) cells, e.g., TEM cells expressing CD45RA (TEMRA) cells, e.g., CD4+ or CD8+ TEMRA cells. In some embodiments, the population of T cells having a memory-like phenotype does not express a senescent marker, e.g., CD57. In some embodiments, the population of T cells having a memory-like phenotype does not express an inhibitory receptor, e.g., OX40, 4-1BB, and/or ICOS.

In some embodiments, the population of T cells having a memory-like phenotype is a population of T cells with CD45RA+ CCR7− CD57−. In some embodiments, the population of T cells having a memory-like phenotype does not express an inhibitory receptor, e.g., OX40, 4-1BB, and/or ICOS.

In some embodiments, the population of T cells having a memory-like phenotype, e.g., as described herein, has increased proliferative capacity, e.g., as compared to a reference cell population, e.g., an otherwise similar population of cells that has not been contacted with an anti-TCRβV antibody or the multispecific or multifunctional molecules as described herein.

In some embodiments, the expansion is at least about 1.1-10 fold expansion (e.g., at least about 1.1, 1.2, 1.3, 1.4, 1.5, 2, 3, 4, 5, 6, 7, 8, 9, or 10 fold expansion).

In some embodiments, expansion of the population of T cells having a memory-like phenotype, e.g., memory effector T cells, e.g., TEM cells, e.g., TEMRA cells, e.g., CD4+ or CD8+ TEMRA cells, is compared to expansion of a similar population of cells with an antibody that binds to: a CD3 molecule, e.g., CD3 epsilon (CD3e) molecule; or a TCR alpha (TCRα) molecule.

In some embodiments, the population of expanded T cells having a memory-like phenotype, e.g., T effector memory cells, comprises cells T cells, e.g., CD3+, CD8+ or CD4+ T cells. In some embodiments, the population of expanded T cells having a memory-like phenotype, T effector memory cells, comprises CD3+ and CD8+ T cells. In some embodiments, the population of expanded T cells having a memory-like phenotype. e.g., T effector memory cells comprises CD3+ and CD4+ T cells.

In some embodiments, the population of expanded T cells having a memory-like phenotype, T effector memory (TEM) cells, comprises cells T cells, e.g., CD3+, CD8+ or CD4+ T cells, which express or re-express, CD45RA, e.g., CD45RA+. In some embodiments, the population comprises TEM cells expressing CD45RA, e.g., TEMRA cells. In some embodiments, expression of CD45RA on TEMRA cells, e.g., CD4+ or CD8+ TEMRA cells, can be detected by a method as described herein, e.g., flow cytometry.

In some embodiments, the population of T cells having a memory-like phenotype, e.g., TEMRA cells have low or no expression of CCR7, e.g., CCR7− or CCR7 low. In some embodiments, expression of CCR7 on TEMRA cells cannot be detected by a method as described herein, e.g., flow cytometry.

In some embodiments, the population of T cells having a memory-like phenotype, e.g., TEMRA cells express CD95, e.g., CD95+. In some embodiments, expression of CD95 on TEMRA cells can be detected by a method as described herein, e.g., flow cytometry.

In some embodiments, the population of T cells having a memory-like phenotype, e.g., TEMRA cells express CD45RA, e.g., CD45RA+, have low or no expression of CCR7, e.g., CCR7− or CCR7 low, and express CD95, e.g., CD95+. In some embodiments, the population of T cells having a memory-like phenotype, e.g., TEMRA cells can be identified as CD45RA+, CCR7− and CD95+ cells. In some embodiments, the population of T cells having a memory-like phenotype, e.g., TEMRA cells comprise CD3+, CD4+ or CD8+ T cells (e.g., CD3+ T cells, CD3+CD8+ T cells, or CD3+CD4+ T cells).

In some embodiments, the population of T cells having a memory-like phenotype does not express a senescent marker, e.g., CD57.

In some embodiments, the population of T cells having a memory-like phenotype does not express an inhibitory receptor, e.g., OX40, 4-1BB, and/or ICOS.

In some embodiments, binding of the multifunctional polypeptide molecule as described herein results in expansion, e.g., at least about 1.1-50 fold expansion (e.g., at least about 1.5-40 fold, 2-35 fold, 3-30 fold, 5-25 fold, 8-20 fold, or 10-15 fold expansion), of a subpopulation of T cells. In some embodiments, the multifunctional polypeptide molecule as described herein-activated (e.g., expanded) subpopulation of T cells resemble TEMRA cells in high expression of CD45RA and/or low expression of CCR7. In some embodiments, the multifunctional polypeptide molecule as described herein-activated (e.g., expanded) subpopulation of T cells do not display upregulation of the senescence markers CD57 and/or KLRG1. In some embodiments, the multifunctional polypeptide molecule as described herein-activated (e.g., expanded) subpopulation of T cells do not display upregulation of co-stimulatory molecules CD27 and/or CD28. In some embodiments, the multifunctional polypeptide molecule as described herein-activated (e.g., expanded) subpopulation of T cells are highly proliferative. In some embodiments, the multifunctional polypeptide molecule as described herein-activated (e.g., expanded) subpopulation of T cells secrete IL-2. In some embodiments, expression of surface markers on T cells can be detected by a method as described herein, e.g., flow cytometry. In some embodiments, the proliferative capability of T cells can be detected by a method as described herein, e.g., a method described in Example 4. In some embodiments, cytokine expression of T cells can be detected by a method as described herein. In some embodiments, the expansion is at least about 1.1-10 fold expansion (e.g., at least about 1.1, 1.2, 1.3, 1.4, 1.5, 2, 3, 4, 5, 6, 7, 8, 9, or 10 fold expansion). In some embodiments, the expansion is compared to expansion of a similar population of cells with an antibody that binds to a CD3 molecule, e.g., CD3 epsilon (CD3e) molecule; or a TCR alpha (TCRα) molecule.

In some embodiments, binding of the multifunctional polypeptide molecule as described herein results in proliferation, e.g., expansion, e.g., at least about 1.1-50 fold expansion (e.g., at least about 1.5-40 fold, 2-35 fold, 3-30 fold, 5-25 fold, 8-20 fold, or 10-15 fold expansion), of a population of Natural Killer (NK) cells. In some embodiments, the expansion of NK cells is at least about 1.1-30 fold expansion (e.g., at least about 1.1, 1.2, 1.3, 1.4, 1.5, 2, 3, 4, 5, 6, 7, 8, 9, 10, 15, 20, 25, or at least about 1.1-5, 5-10, 10-15, 15-20, 20-25, or 25-30 fold expansion). In some embodiments, the expansion of NK cells is measure by an assay of Example 4. In some embodiments, the expansion of NK cells by, e.g., binding of, the multifunctional polypeptide molecule as described herein is compared to expansion of an otherwise similar population not contacted with the multifunctional polypeptide molecule as described herein.

In some embodiments, binding of the multifunctional polypeptide molecule as described herein results in cell killing, e.g., target cell killing, e.g. cancer cell killing. In some embodiments, the cancer cell is a hematological cancer cell or a solid tumor cell. In some embodiments, the cancer cell is a multiple myeloma cell. In some embodiments, binding of the multifunctional polypeptide molecule as described herein results in cell killing in vitro or in vivo. In some embodiments, cell killing is measured by an assay of Example 4.

In some embodiments, binding of the multifunctional polypeptide molecule as described herein to a TCRβV region results in an increase or decrease of at least 2, 5, 10, 20, 50, 100, 200, 300, 400, 500, 600, 700, 800, 900, 1000, or 2000 fold, or at least 2-2000 fold (e.g., 5-1000, 10-900, 20-800, 50-700, 100-600, 200-500, or 300-400 fold) of any of the activities described herein compared the activity of 16G8 or TM23 murine antibody, or a humanized version thereof as described in U.S. Pat. No. 5,861,155.

In some embodiments, the method comprises expanding, e.g., increasing the number of, an immune cell population in the subject. In some embodiments, provided herein is a method of expanding, e.g., increasing the number of, an immune cell population comprising, contacting the immune cell population with an effective amount of the multifunctional polypeptide molecule as described herein. In some embodiments, the expansion occurs in vivo or ex vivo (e.g., in vitro).

In some embodiments, provided herein is a method of expanding, e.g., increasing the number of, an immune cell population comprising, contacting the immune cell population with a multifunctional polypeptide molecule as described herein comprising an antibody molecule, e.g., humanized antibody molecule, which binds, e.g., specifically binds, to a T cell receptor beta variable chain (TCRβV) region (e.g., anti-TCRαV antibody molecule) or specifically binds, to a T cell receptor beta variable chain (TCRαV) region (e.g., anti-TCRαV antibody molecule), thereby expanding the immune cell population. In some embodiments, the expansion occurs in vivo or ex vivo (e.g., in vitro).

In some embodiments, provided herein is a method of expanding a population of immune effector cells from a subject having a cancer, the method comprising: (i) isolating a biological sample comprising a population of immune effector cells from the subject; e.g., a peripheral blood sample, biopsy sample, or bone marrow sample; (ii) acquiring a value of the status of one or more TCRβV molecules or TCRαV molecules for the subject, e.g., in the biological sample from the subject, wherein said value comprises a measure of the presence of, e.g., level or activity of, a TCRβV molecule or a TCRαV molecule in a sample from the subject compared to a reference value, e.g., a sample from a health subject, wherein a value that is higher, e.g., increased, in the subject relative to the reference, e.g., healthy subject, is indicative of the presence of cancer in the subject, and (iii) contacting the biological sample comprising a population of immune effector cells with the multifunctional polypeptide molecule as described herein.

In some embodiments, the method further comprises administering the population of immune effector cells contacted with the multifunctional polypeptide molecule as described herein to the subject.

In some embodiments, a higher. e.g., increased, level or activity of one or more TCRβV molecules or TCRαV molecules in a subject, e.g., in a sample from a subject, is indicative of a bias, e.g., a preferential expansion, e.g., clonal expansion, of T cells expressing said one or more TCRβV molecules or TCRαV molecules in the subject.

Accordingly, provided herein are, inter alia, multispecific or multifunctional molecules comprising TCRβV-binding or TCRαV-binding moieties as described herein (e.g., multispecific or multifunctional antibody molecules) that comprise anti-TCRβV antibody molecules or anti-TCRαV antibody molecules, nucleic acids encoding the same, methods of producing the aforesaid molecules, pharmaceutical compositions comprising aforesaid molecules, and methods of treating a disease or disorder, e.g., cancer, using the aforesaid molecules. The antibody molecules and pharmaceutical compositions as described herein can be used (alone or in combination with other agents or therapeutic modalities) to treat, prevent and/or diagnose disorders and conditions, e.g., cancer, e.g., as described herein.

### Exemplary Multifunctional Polypeptide Molecule

In some embodiments, the first agent and/or the second agent is a multifunctional molecule, a multispecific molecule, a multifunctional polypeptide molecule, or a multispecific polypeptide molecule.

In some embodiments, any of the compositions and methods described herein can be used to expand an immune cell population. An immune cell provided herein includes an immune cell derived from a hematopoietic stem cell or an immune cell derived from a non-hematopoietic stem cell, e.g., by differentiation or de-differentiation.

In some embodiments, the multifunctional polypeptide molecule comprises: (i) a first polypeptide chain comprises a VH comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3445, wherein the VH is linked to a heavy chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649, wherein the heavy chain constant region comprises the first Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3447, (ii) a second polypeptide chain comprises a cytokine molecule, wherein the cytokine molecule is linked to the second Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a linker comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, wherein the cytokine molecule comprises IL-2 or functional variant thereof comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2270, and (iii) a third polypeptide chain comprises a VL comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3446, wherein the VL is linked to a light chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide chain comprises a VH comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3448, wherein the VH is linked to a heavy chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649, wherein the heavy chain constant region comprises the first Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3447, (ii) a second polypeptide chain comprises a cytokine molecule, wherein the cytokine molecule is linked to the second Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a linker comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, wherein the cytokine molecule comprises IL-2 or functional variant thereof comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2270, and (iii) a third polypeptide chain comprises a VL comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3449, wherein the VL is linked to a light chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide chain comprises a VH comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3450, wherein the VH is linked to a heavy chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649, wherein the heavy chain constant region comprises the first Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3447, (ii) a second polypeptide chain comprises a cytokine molecule, wherein the cytokine molecule is linked to the second Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a linker comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, wherein the cytokine molecule comprises IL-2 or functional variant thereof comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2270, and
(iii) a third polypeptide chain comprises a VL comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3451, wherein the VL is linked to a light chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) the first polypeptide chain comprises a VH comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3450, wherein the VH is linked to a heavy chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649, wherein the heavy chain constant region comprises the first Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3447, (ii) a second polypeptide chain comprises a cytokine molecule, wherein the cytokine molecule is linked to the second Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3454 or the sequence of SEQ ID NO: 3501 via a linker comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, wherein the cytokine molecule comprises IL-2 or functional variant thereof comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2270, and (iii) a third polypeptide chain comprises a VL comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3451, wherein the VL is linked to a light chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide chain comprises a VH comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346, wherein the VH is linked to a heavy chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3452 or the sequence of SEQ ID NO: 3504, wherein the heavy chain constant region comprises the first Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3453 or the sequence of SEQ ID NO: 3505, (ii) a second polypeptide chain comprises a cytokine molecule, wherein the cytokine molecule is linked to the second Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3454 or the sequence of SEQ ID NO: 3501 via a linker comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, wherein the cytokine molecule comprises IL-2 or functional variant thereof comprising a sequence having at least 50%, 55%, 60%, 65%, 70%, 75%, 80%, 85%, 90%, 95%, 97%, 98%, 99%, 99.5%, 99.6%, 99.7%, 99.80, or 99.9% sequence identity to the sequence of SEQ ID NO: 2270, and (iii) a third polypeptide chain comprises a VL comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1349, wherein the VL is linked to a light chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide chain comprises a VH comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1196, wherein the VH is linked to a heavy chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649, wherein the heavy chain constant region comprises the first Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3447, (ii) a second polypeptide chain comprises a cytokine molecule, wherein the cytokine molecule is linked to the second Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a linker comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, wherein the cytokine molecule comprises IL-2 or functional variant thereof comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2270, and
(iii) a third polypeptide chain comprises a VL comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1195, wherein the VL is linked to a light chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide chain comprises a VH comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3455, wherein the VH is linked to a heavy chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649, wherein the heavy chain constant region comprises the first Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3447, (ii) a second polypeptide chain comprises a cytokine molecule, wherein the cytokine molecule is linked to the second Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a linker comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, wherein the cytokine molecule comprises IL-2 or functional variant thereof comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2270, and (iii) a third polypeptide chain comprises a VL comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3456, wherein the VL is linked to a light chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) the first polypeptide chain comprises a VH comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3470, wherein the VH is linked to a heavy chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649, wherein the heavy chain constant region comprises the first Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3447, (ii) a second polypeptide chain comprises a cytokine molecule, wherein the cytokine molecule is linked to the second Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a linker comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, wherein the cytokine molecule comprises IL-2 or functional variant thereof comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2270, and (iii) a third polypeptide chain comprises a VL comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3471, wherein the VL is linked to a light chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide chain comprises a VH comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346, wherein the VH is linked to a heavy chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3452 or the sequence of SEQ ID NO: 3504, wherein the heavy chain constant region comprises the first Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3453 or the sequence of SEQ ID NO: 3505, (ii) a second polypeptide chain comprises a cytokine molecule, wherein the cytokine molecule is linked to the second Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a linker comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, wherein the cytokine molecule comprises IL-15 or functional variant thereof comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2170, wherein the IL-15 or functional variant thereof is covalently linked to an IL15Ralpha dimerizing domain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of the SEQ ID NO: 3472, and (iii) a third polypeptide chain comprises a VL comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1349, wherein the VL is linked to a light chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide chain comprises a VH comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346, wherein the VH is linked to a heavy chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3452 or the sequence of SEQ ID NO: 3504, wherein the heavy chain constant region comprises the first Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3453 or the sequence of SEQ ID NO: 3505, (ii) a second polypeptide chain comprises a cytokine molecule, wherein the cytokine molecule is linked to the second Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a linker comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, wherein the cytokine molecule comprises IL-15 or functional variant thereof comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2170, wherein the IL-15 or functional variant thereof is covalently linked to an IL15Ralpha dimerizing domain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of the SEQ ID NO: 3472, and (iii) a third polypeptide chain comprises a VL comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3475, wherein the VL is linked to a light chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide chain comprises a VH comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346, wherein the VH is linked to a heavy chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3452 or the sequence of SEQ ID NO: 3504, wherein the heavy chain constant region comprises the first Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3453 or the sequence of SEQ ID NO: 3505, (ii) a second polypeptide chain comprises a cytokine molecule, wherein the cytokine molecule is linked to the second Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a linker comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, wherein the cytokine molecule comprises IL-15 or functional variant thereof comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2170, wherein the IL-15 or functional variant thereof is covalently linked to an IL15Ralpha dimerizing domain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of the SEQ ID NO: 3472, and (iii) a third polypeptide chain comprises a VL comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3476, wherein the VL is linked to a light chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide chain comprises a VH comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3448, wherein the VH is linked to a heavy chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649, wherein the heavy chain constant region comprises the first Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3447, (ii) a second polypeptide chain comprises a cytokine molecule, wherein the cytokine molecule is linked to the second Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a linker comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, wherein the cytokine molecule comprises IL-2 or functional variant thereof comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2270, and (iii) a third polypeptide chain comprises a VL comprising a sequence having at least 50%, 55%, 60%, 65%, 70%, 750, 80%, 85%, 90%, 95%, 97%, 98%, 99%, 99.5%, 99.6%, 99.7%, 99.8%, or 99.9% sequence identity to the sequence of SEQ ID NO: 3449, wherein the VL is linked to a light chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide chain comprises a VH comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3450, wherein the VH is linked to a heavy chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649, wherein the heavy chain constant region comprises the first Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3447, (ii) a second polypeptide chain comprises a cytokine molecule, wherein the cytokine molecule is linked to the second Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a linker comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, wherein the cytokine molecule comprises IL-2 or functional variant thereof comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2270, and (ii) a third polypeptide chain comprises a VL comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3451, wherein the VL is linked to a light chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide chain comprises a VH comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3450, wherein the VH is linked to a heavy chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649, wherein the heavy chain constant region comprises the first Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3447, (ii) a second polypeptide chain comprises a cytokine molecule, wherein the cytokine molecule is linked to the second Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3454 or the sequence of SEQ ID NO: 3501 via a linker comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, wherein the cytokine molecule comprises IL-2 or functional variant thereof comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2270, and (iii) a third polypeptide chain comprises a VL comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3451, wherein the VL is linked to a light chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide chain comprises a VH comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346, wherein the VH is linked to a heavy chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3452 or the sequence of SEQ ID NO: 3504, wherein the heavy chain constant region comprises the first Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3453 or the sequence of SEQ ID NO: 3505, (ii) a second polypeptide chain comprises a cytokine molecule, wherein the cytokine molecule is linked to the second Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3454 or the sequence of SEQ ID NO: 3501 via a linker comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, wherein the cytokine molecule comprises IL-2 or functional variant thereof comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2270, and (iii) a third polypeptide chain comprises a VL comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1349, wherein the VL is linked to a light chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide chain comprises a VH comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1196, wherein the VH is linked to a heavy chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649, wherein the heavy chain constant region comprises the first Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3447, (ii) a second polypeptide chain comprises a cytokine molecule, wherein the cytokine molecule is linked to the second Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a linker comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, wherein the cytokine molecule comprises IL-2 or functional variant thereof comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2270, and (iii) a third polypeptide chain comprises a VL comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1195, wherein the VL is linked to a light chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide chain comprises a VH comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3455, wherein the VH is linked to a heavy chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649, wherein the heavy chain constant region comprises the first Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3447, (ii) a second polypeptide chain comprises a cytokine molecule, wherein the cytokine molecule is linked to the second Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a linker comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, wherein the cytokine molecule comprises IL-2 or functional variant thereof comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2270, and (iii) a third polypeptide chain comprises a VL comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3456, wherein the VL is linked to a light chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide chain comprises a VH comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3470, wherein the VH is linked to a heavy chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3649, wherein the heavy chain constant region comprises the first Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3447, (ii) a second polypeptide chain comprises a cytokine molecule, wherein the cytokine molecule is linked to the second Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a linker comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, wherein the cytokine molecule comprises IL-2 or functional variant thereof comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2270, and (iii) a third polypeptide chain comprises a VL comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3471, wherein the VL is linked to a light chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide chain comprises a VH comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346, wherein the VH is linked to a heavy chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9/a sequence identity to the sequence of SEQ ID NO: 3452 or the sequence of SEQ ID NO: 3504, wherein the heavy chain constant region comprises the first Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3453 or the sequence of SEQ ID NO: 3505, (ii) a second polypeptide chain comprises a cytokine molecule, wherein the cytokine molecule is linked to the second Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a linker comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, wherein the cytokine molecule comprises IL-15 or functional variant thereof comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2170, wherein the IL-15 or functional variant thereof is covalently linked to an IL15Ralpha dimerizing domain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of the SEQ ID NO: 3472, and (iii) a third polypeptide chain comprises a VL comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1349, wherein the VL is linked to a light chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the multifunctional polypeptide molecule comprises: (i) a first polypeptide chain comprises a VH comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 1346, wherein the VH is linked to a heavy chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3452 or the sequence of SEQ ID NO: 3504, wherein the heavy chain constant region comprises the first Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3453 or the sequence of SEQ ID NO: 3505, (ii) a second polypeptide chain comprises a cytokine molecule, wherein the cytokine molecule is linked to the second Fc region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3648 via a linker comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3308, wherein the cytokine molecule comprises IL-15 or functional variant thereof comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 2170, wherein the IL-15 or functional variant thereof is covalently linked to an IL15Ralpha dimerizing domain comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of the SEQ ID NO: 3472, and (iii) a third polypeptide chain comprises a VL comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3475, wherein the VL is linked to a light chain constant region comprising a sequence having at least 50%, 55%, 60%, 65%, 70%, 75%, 80%, 85%, 90%, 95%, 97%, 98%, 99%, 99.5%, 99.6%, 99.7%, 99.8%, or 99.9% sequence identity to the sequence of SEQ ID NO: 3644.

In some embodiments, the first binder comprises a VH comprising:
(i) a HC CDR1 comprising the sequence of SEQ ID NO: 3341, a HC CDR2 comprising the sequence of SEQ ID NO: 3342, and/or a HC CDR3 comprising the sequence of SEQ ID NO: 3343;
(ii) a HC CDR1 comprising the sequence of SEQ ID NO: 3321, a HC CDR2 comprising the sequence of SEQ ID NO: 3322, and/or a HC CDR3 comprising the sequence of SEQ ID NO: 3323;
(iii) a HC CDR1 comprising the sequence of SEQ ID NO: 3351, a HC CDR2 comprising the sequence of SEQ ID NO: 3352, and/or a HC CDR3 comprising the sequence of SEQ ID NO: 3353;
(iv) a HC CDR1 comprising the sequence of SEQ ID NO: 1190, a HC CDR2 comprising the sequence of SEQ ID NO: 1186, and/or a HC CDR3 comprising the sequence of SEQ ID NO: 1187;
(v) a HC CDR1 comprising the sequence of SEQ ID NO: 3371, a HC CDR2 comprising the sequence of SEQ ID NO: 3372, and/or a HC CDR3 comprising the sequence of SEQ ID NO: 3373;
(vi) a HC CDR1 comprising the sequence of SEQ ID NO: 3381, a HC CDR2 comprising the sequence of SEQ ID NO: 3382, and/or a HC CDR3 comprising the sequence of SEQ ID NO: 3383;
(vii) a HC CDR1 comprising the sequence of SEQ ID NO: 3391, a HC CDR2 comprising the sequence of SEQ ID NO: 3392, and/or a HC CDR3 comprising the sequence of SEQ ID NO: 3393;
(viii) a HC CDR1 comprising the sequence of SEQ ID NO: 3401, a HC CDR2 comprising the sequence of SEQ ID NO: 3402, and/or a HC CDR3 comprising the sequence of SEQ ID NO: 3403;
(ix) a HC CDR1 comprising the sequence of SEQ ID NO: 3351, SEQ ID NO: 3421, or SEQ ID NO: 3702, a HC CDR2 comprising the sequence of SEQ ID NO: 3352, and a HC CDR3 comprising the sequence of SEQ ID NO: 3353; or
(x) a HC CDR1 comprising the sequence of SEQ ID NO: 3411, a HC CDR2 comprising the sequence of SEQ ID NO: 3412, and a HC CDR3 comprising the sequence of SEQ ID NO: 3413.

In some embodiments, the first binder comprises a VL comprising:
(i) a LC CDR1 comprising the sequence of SEQ ID NO: 3344, a LC CDR2 comprising the sequence of SEQ ID NO: 3345, and/or a LC CDR3 comprising the sequence of SEQ ID NO: 3346;
(ii) a LC CDR1 comprising the sequence of SEQ ID NO: 3324, a LC CDR2 comprising the sequence of SEQ ID NO: 3325, and/or a LC CDR3 comprising the sequence of SEQ ID NO: 3326;
(iii) a LC CDR1 comprising the sequence of SEQ ID NO: 3354, a LC CDR2 comprising the sequence of SEQ ID NO: 3355, and/or a LC CDR3 comprising the sequence of SEQ ID NO: 3356;
(iv) a LC CDR1 comprising the sequence of SEQ ID NO: 1191, a LC CDR2 comprising the sequence of SEQ ID NO: 1192, and/or a LC CDR3 comprising the sequence of SEQ ID NO: 1193;
(v) a LC CDR1 comprising the sequence of SEQ ID NO: 3374, a LC CDR2 comprising the sequence of SEQ ID NO: 3375, and/or a LC CDR3 comprising the sequence of SEQ ID NO: 3376;
(vi) a LC CDR1 comprising the sequence of SEQ ID NO: 3384, a LC CDR2 comprising the sequence of SEQ ID NO: 3325, and/or a LC CDR3 comprising the sequence of SEQ ID NO: 3386;
(vii) a LC CDR1 comprising the sequence of SEQ ID NO: 3394, a LC CDR2 comprising the sequence of SEQ ID NO: 3395, and/or a LC CDR3 comprising the sequence of SEQ ID NO: 3396;
(viii) a LC CDR1 comprising the sequence of SEQ ID NO: 3404, a LC CDR2 comprising the sequence of SEQ ID NO: 3405, and/or a LC CDR3 comprising the sequence of SEQ ID NO: 3406;
(ix) a LC CDR1 comprising the sequence of SEQ ID NO: 3414, a LC CDR2 comprising the sequence of SEQ ID NO: 3415, and/or a LC CDR3 comprising the sequence of SEQ ID NO: 3416; or
(x) a LC CDR1 comprising the sequence of SEQ ID NO: 3354, a LC CDR2 comprising the sequence of SEQ ID NO: 3443, and/or a LC CDR3 comprising the sequence of SEQ ID NO: 3356.

In some embodiments, the first binder comprises:
(i) a VH comprising a HC CDR1 comprising the sequence of SEQ ID NO: 3341, a HC CDR2 comprising the sequence of SEQ ID NO: 3342, a HC CDR3 comprising the sequence of SEQ ID NO: 3343, and/or a VL comprising a LC CDR1 comprising the sequence of SEQ ID NO: 3344, a LC CDR2 comprising the sequence of SEQ ID NO: 3345, and a LC CDR3 comprising the sequence of SEQ ID NO: 3346;
(ii) a VH comprising a HC CDR1 comprising the sequence of SEQ ID NO: 3321, a HC CDR2 comprising the sequence of SEQ ID NO: 3322, a HC CDR3 comprising the sequence of SEQ ID NO: 3323, and/or a VL comprising a LC CDR1 comprising the sequence of SEQ ID NO: 3324, a LC CDR2 comprising the sequence of SEQ ID NO: 3325, and a LC CDR3 comprising the sequence of SEQ ID NO: 3326;
(iii) a VH comprising a HC CDR1 comprising the sequence of SEQ ID NO: 3351, a HC CDR2 comprising the sequence of SEQ ID NO: 3352, a HC CDR3 comprising the sequence of SEQ ID NO: 3353, and/or a VL comprising a LC CDR1 comprising the sequence of SEQ ID NO: 3354, a LC CDR2 comprising the sequence of SEQ ID NO: 3355, and a LC CDR3 comprising the sequence of SEQ ID NO: 3356;
(iv) a VH comprising a HC CDR1 comprising the sequence of SEQ ID NO: 1190, a HC CDR2 comprising the sequence of SEQ ID NO: 1186, a HC CDR3 comprising the sequence of SEQ ID NO: 1187, and/or a VL comprising a LC CDR1 comprising the sequence of SEQ ID NO: 1191, a LC CDR2 comprising the sequence of SEQ ID NO: 1192, and a LC CDR3 comprising the sequence of SEQ ID NO: 1193;
(v) a VH comprising a HC CDR1 comprising the sequence of SEQ ID NO: 3371, a HC CDR2 comprising the sequence of SEQ ID NO: 3372, a HC CDR3 comprising the sequence of SEQ ID NO: 3373, and/or a VL comprising a LC CDR1 comprising the sequence of SEQ ID NO: 3374, a LC CDR2 comprising the sequence of SEQ ID NO: 3375, and a LC CDR3 comprising the sequence of SEQ ID NO: 3376;
(vi) a VH comprising a HC CDR1 comprising the sequence of SEQ ID NO: 3381, a HC CDR2 comprising the sequence of SEQ ID NO: 3382, a HC CDR3 comprising the sequence of SEQ ID NO: 3383, and/or a VL comprising a LC CDR1 comprising the sequence of SEQ ID NO: 3384, a LC CDR2 comprising the sequence of SEQ ID NO: 3325, and a LC CDR3 comprising the sequence of SEQ ID NO: 3386;
(vii) a VH comprising a HC CDR1 comprising the sequence of SEQ ID NO: 3391, a HC CDR2 comprising the sequence of SEQ ID NO: 3392, a HC CDR3 comprising the sequence of SEQ ID NO: 3393, and/or a VL comprising a LC CDR1 comprising the sequence of SEQ ID NO: 3394, a LC CDR2 comprising the sequence of SEQ ID NO: 3395, and a LC CDR3 comprising the sequence of SEQ ID NO: 3396;
(viii) a VH comprising a HC CDR1 comprising the sequence of SEQ ID NO: 3401, a HC CDR2 comprising the sequence of SEQ ID NO: 3402, a HC CDR3 comprising the sequence of SEQ ID NO: 3403, and/or a VL comprising a LC CDR1 comprising the sequence of SEQ ID NO: 3404, a LC CDR2 comprising the sequence of SEQ ID NO: 3405, and a LC CDR3 comprising the sequence of SEQ ID NO: 3406;
(ix) a VH comprising a HC CDR1 comprising the sequence of SEQ ID NO: 3411, a HC CDR2 comprising the sequence of SEQ ID NO: 3412, a HC CDR3 comprising the sequence of SEQ ID NO: 3413, and/or a VL comprising a LC CDR1 comprising the sequence of SEQ ID NO: 3414, a LC CDR2 comprising the sequence of SEQ ID NO: 3415, and a LC CDR3 comprising the sequence of SEQ ID NO: 3416;
(x) a VH comprising a HC CDR1 comprising the sequence of SEQ ID NO: 3351, SEQ ID NO: 3421, or SEQ ID NO: 3702, a HC CDR2 comprising the sequence of SEQ ID NO: 3352, a HC CDR3 comprising the sequence of SEQ ID NO: 3353, and/or a VL comprising a LC CDR1 comprising the sequence of SEQ ID NO: 3354, a LC CDR2 comprising the sequence of SEQ ID NO: 3355, and a LC CDR3 comprising the sequence of SEQ ID NO: 3356; or
(xi) a VH comprising a HC CDR1 comprising the sequence of SEQ ID NO: 3351, SEQ ID NO: 3421, or SEQ ID NO: 3702, a HC CDR2 comprising the sequence of SEQ ID NO: 3352, a HC CDR3 comprising the sequence of SEQ ID NO: 3353, and/or a VL comprising a LC CDR1 comprising the sequence of SEQ ID NO: 3354, a LC CDR2 comprising the sequence of SEQ ID NO: 3443, and a LC CDR3 comprising the sequence of SEQ ID NO: 3356.

In some embodiments, the first binder comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, or 99.9% sequence identity to any one sequence selected from the group consisting of: SEQ ID NO: 3445, SEQ ID NO: 3448, SEQ ID NO: 3450, SEQ ID NO: 1346, SEQ ID NO: 1196, SEQ ID NO: 3455, SEQ ID NO: 3457, SEQ ID NO: 3463, SEQ ID NO: 3465, and SEQ ID NO: 3470.

In some embodiments, the first binder comprises a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to any one sequence selected from the group consisting of: SEQ ID NO: 3446, SEQ ID NO: 3449, SEQ ID NO: 3451, SEQ ID NO: 1349, SEQ ID NO:1195, SEQ ID NO: 3456, SEQ ID NO: 3458, SEQ ID NO: 3464, SEQ ID NO: 3466, SEQ ID NO: 3471, SEQ ID NO: 3475, and SEQ ID NO: 3476.

In some embodiments, the first binder comprises:
(i) a VH comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to any one sequence selected from the group consisting of: SEQ ID NO: 3445, SEQ ID NO: 3448, SEQ ID NO: 3450, SEQ ID NO: 1346, SEQ ID NO: 1196, SEQ ID NO: 3455, SEQ ID NO: 3457, SEQ ID NO: 3463, SEQ ID NO: 3465, and SEQ ID NO: 3470; and/or
(ii) a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to any one sequence selected from the group consisting of: SEQ ID NO: 3446, SEQ ID NO: 3449, SEQ ID NO: 3451, SEQ ID NO: 1349, SEQ ID NO:1195, SEQ ID NO: 3456, SEQ ID NO: 3458, SEQ ID NO: 3464, SEQ ID NO: 3466, SEQ ID NO: 3471, SEQ ID NO: 3475, and SEQ ID NO: 3476.

In some embodiments, the first binder comprises:
(i) a VH comprising any one sequence selected from the group consisting of: SEQ ID NO: 3445, SEQ ID NO: 3448, SEQ ID NO: 3450, SEQ ID NO: 1346, SEQ ID NO: 1196, SEQ ID NO: 3455, SEQ ID NO: 3457, SEQ ID NO: 3463, SEQ ID NO: 3465, and SEQ ID NO: 3470; and/or
(ii) a VL comprising any one sequence selected from the group consisting of: SEQ ID NO: 3446, SEQ ID NO: 3449, SEQ ID NO: 3451, SEQ ID NO: 1349, SEQ ID NO: 1195, SEQ ID NO: 3456, SEQ ID NO: 3458, SEQ ID NO: 3464, SEQ ID NO: 3466, SEQ ID NO: 3471, SEQ ID NO: 3475, and SEQ ID NO: 3476.

In some embodiments, the first binder comprises:
(i) a VH comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3445, and/or a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3446;
(ii) a VH comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3448, and/or a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3449;
(iii) a VH comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3450, and/or a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3451;
(iv) a VH comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 1346, and/or a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 1349;
(v) a VH comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 1196, and/or a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 1195;
(vi) a VH comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3455, and/or a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3456;
(vii) a VH comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3457, and/or a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3458;
(viii) a VH comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3463, and/or a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3464;
(ix) a VH comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3465, and/or a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3466;
(x) a VH comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3470, and/or a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3471;
(xi) a VH comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 1346, and/or a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3475; or
(xii) a VH comprising a sequence having 30%, 35%, 40%, 45%, 50%, 55%1, 60%, 65%, 70%, 75%, 80%, 85%, 90%, 95%, 96%, 97%, 98%, 99%, 99.5%, 99.9%, or 100% sequence identity to the sequence of SEQ ID NO: 1346, and/or a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3476.

In some embodiments, the first binder comprises:
(i) a VH comprising the sequence of SEQ ID NO: 3445, and/or a VL comprising the sequence of SEQ ID NO: 3446;
(ii) a VH comprising the sequence of SEQ ID NO: 3448, and/or a VL comprising the sequence of SEQ ID NO: 3449;
(iii) a VH comprising the sequence of SEQ ID NO: 3450, and/or a VL comprising the sequence of SEQ ID NO: 3451;
(iv) a VH comprising the sequence of SEQ ID NO: 1346, and/or a VL comprising the sequence of SEQ ID NO: 1349;
(v) a VH comprising the sequence of SEQ ID NO: 1196, and/or a VL comprising the sequence of SEQ ID NO: 1195;
(vi) a VH comprising the sequence of SEQ ID NO: 3455, and/or a VL comprising the sequence of SEQ ID NO: 3456;
(vii) a VH comprising the sequence of SEQ ID NO: 3457, and/or a VL comprising the sequence of SEQ ID NO: 3458;
(viii) a VH comprising the sequence of SEQ ID NO: 3463, and/or a VL comprising the sequence of SEQ ID NO: 3464;
(ix) a VH comprising the sequence of SEQ ID NO: 3465, and/or a VL comprising the sequence of SEQ ID NO: 3466;
(x) a VH comprising the sequence of SEQ ID NO: 3470, and/or a VL comprising the sequence of SEQ ID NO: 3471;
(xi) a VH comprising the sequence of SEQ ID NO: 1346, and/or a VL comprising the sequence of SEQ ID NO: 3475; or
(xii) a VH comprising the sequence of SEQ ID NO: 1346, and/or a VL comprising the sequence of SEQ ID NO: 3476.

In some embodiments, the second binder comprises a VH comprising:
(i) a HC CDR1 comprising the sequence of SEQ ID NO: 3341, a HC CDR2 comprising the sequence of SEQ ID NO: 3342, and/or a HC CDR3 comprising the sequence of SEQ ID NO: 3343;
(ii) a HC CDR1 comprising the sequence of SEQ ID NO: 3321, a HC CDR2 comprising the sequence of SEQ ID NO: 3322, and/or a HC CDR3 comprising the sequence of SEQ ID NO: 3323;
(iii) a HC CDR1 comprising the sequence of SEQ ID NO: 3351, a HC CDR2 comprising the sequence of SEQ ID NO: 3352, and/or a HC CDR3 comprising the sequence of SEQ ID NO: 3353;
(iv) a HC CDR1 comprising the sequence of SEQ ID NO: 1190, a HC CDR2 comprising the sequence of SEQ ID NO: 1186, and/or a HC CDR3 comprising the sequence of SEQ ID NO: 1187;
(v) a HC CDR1 comprising the sequence of SEQ ID NO: 3371, a HC CDR2 comprising the sequence of SEQ ID NO: 3372, and/or a HC CDR3 comprising the sequence of SEQ ID NO: 3373;
(vi) a HC CDR1 comprising the sequence of SEQ ID NO: 3381, a HC CDR2 comprising the sequence of SEQ ID NO: 3382, and/or a HC CDR3 comprising the sequence of SEQ ID NO: 3383;
(vii) a HC CDR1 comprising the sequence of SEQ ID NO: 3391, a HC CDR2 comprising the sequence of SEQ ID NO: 3392, and/or a HC CDR3 comprising the sequence of SEQ ID NO: 3393;
(viii) a HC CDR1 comprising the sequence of SEQ ID NO: 3401, a HC CDR2 comprising the sequence of SEQ ID NO: 3402, and/or a HC CDR3 comprising the sequence of SEQ ID NO: 3403;
(ix) a HC CDR1 comprising the sequence of SEQ ID NO: 3351, SEQ ID NO: 3421, or SEQ ID NO: 3702, a HC CDR2 comprising the sequence of SEQ ID NO: 3352, and a HC CDR3 comprising the sequence of SEQ ID NO: 3353; or
(x) a HC CDR1 comprising the sequence of SEQ ID NO: 3411, a HC CDR2 comprising the sequence of SEQ ID NO: 3412, and a HC CDR3 comprising the sequence of SEQ ID NO: 3413.

In some embodiments, the second binder comprises a VL comprising:
(i) a LC CDR1 comprising the sequence of SEQ ID NO: 3344, a LC CDR2 comprising the sequence of SEQ ID NO: 3345, and/or a LC CDR3 comprising the sequence of SEQ ID NO: 3346;
(ii) a LC CDR1 comprising the sequence of SEQ ID NO: 3324, a LC CDR2 comprising the sequence of SEQ ID NO: 3325, and/or a LC CDR3 comprising the sequence of SEQ ID NO: 3326;
(iii) a LC CDR1 comprising the sequence of SEQ ID NO: 3354, a LC CDR2 comprising the sequence of SEQ ID NO: 3355, and/or a LC CDR3 comprising the sequence of SEQ ID NO: 3356;
(iv) a LC CDR1 comprising the sequence of SEQ ID NO: 1191, a LC CDR2 comprising the sequence of SEQ ID NO: 1192, and/or a LC CDR3 comprising the sequence of SEQ ID NO: 1193;
(v) a LC CDR1 comprising the sequence of SEQ ID NO: 3374, a LC CDR2 comprising the sequence of SEQ ID NO: 3375, and/or a LC CDR3 comprising the sequence of SEQ ID NO: 3376;
(vi) a LC CDR1 comprising the sequence of SEQ ID NO: 3384, a LC CDR2 comprising the sequence of SEQ ID NO: 3325, and/or a LC CDR3 comprising the sequence of SEQ ID NO: 3386;
(vii) a LC CDR1 comprising the sequence of SEQ ID NO: 3394, a LC CDR2 comprising the sequence of SEQ ID NO: 3395, and/or a LC CDR3 comprising the sequence of SEQ ID NO: 3396;
(viii) a LC CDR1 comprising the sequence of SEQ ID NO: 3404, a LC CDR2 comprising the sequence of SEQ ID NO: 3405, and/or a LC CDR3 comprising the sequence of SEQ ID NO: 3406;
(ix) a LC CDR1 comprising the sequence of SEQ ID NO: 3414, a LC CDR2 comprising the sequence of SEQ ID NO: 3415, and/or a LC CDR3 comprising the sequence of SEQ ID NO: 3416; or
(x) a LC CDR1 comprising the sequence of SEQ ID NO: 3354, a LC CDR2 comprising the sequence of SEQ ID NO: 3443, and/or a LC CDR3 comprising the sequence of SEQ ID NO: 3356.

In some embodiments, the second binder comprises:
(i) a VH comprising a HC CDR1 comprising the sequence of SEQ ID NO: 3341, a HC CDR2 comprising the sequence of SEQ ID NO: 3342, a HC CDR3 comprising the sequence of SEQ ID NO: 3343, and/or a VL comprising a LC CDR1 comprising the sequence of SEQ ID NO: 3344, a LC CDR2 comprising the sequence of SEQ ID NO: 3345, and a LC CDR3 comprising the sequence of SEQ ID NO: 3346;
(ii) a VH comprising a HC CDR1 comprising the sequence of SEQ ID NO: 3321, a HC CDR2 comprising the sequence of SEQ ID NO: 3322, a HC CDR3 comprising the sequence of SEQ ID NO: 3323, and/or a VL comprising a LC CDR1 comprising the sequence of SEQ ID NO: 3324, a LC CDR2 comprising the sequence of SEQ ID NO: 3325, and a LC CDR3 comprising the sequence of SEQ ID NO: 3326.
(iii) a VH comprising a HC CDR1 comprising the sequence of SEQ ID NO: 3351, a HC CDR2 comprising the sequence of SEQ ID NO: 3352, a HC CDR3 comprising the sequence of SEQ ID NO: 3353, and/or a VL comprising a LC CDR1 comprising the sequence of SEQ ID NO: 3354, a LC CDR2 comprising the sequence of SEQ ID NO: 3355, and a LC CDR3 comprising the sequence of SEQ ID NO: 3356;
(iv) a VH comprising a HC CDR1 comprising the sequence of SEQ ID NO: 1190, a HC CDR2 comprising the sequence of SEQ ID NO: 1186, a HC CDR3 comprising the sequence of SEQ ID NO: 1187, and/or a VL comprising a LC CDR1 comprising the sequence of SEQ ID NO: 1191, a LC CDR2 comprising the sequence of SEQ ID NO: 1192, and a LC CDR3 comprising the sequence of SEQ ID NO: 1193;
(v) a VH comprising a HC CDR1 comprising the sequence of SEQ ID NO: 3371, a HC CDR2 comprising the sequence of SEQ ID NO: 3372, a HC CDR3 comprising the sequence of SEQ ID NO: 3373, and/or a VL comprising a LC CDR1 comprising the sequence of SEQ ID NO: 3374, a LC CDR2 comprising the sequence of SEQ ID NO: 3375, and a LC CDR3 comprising the sequence of SEQ ID NO: 3376;
(vi) a VH comprising a HC CDR1 comprising the sequence of SEQ ID NO: 3381, a HC CDR2 comprising the sequence of SEQ ID NO: 3382, a HC CDR3 comprising the sequence of SEQ ID NO: 3383, and/or a VL comprising a LC CDR1 comprising the sequence of SEQ ID NO: 3384, a LC CDR2 comprising the sequence of SEQ ID NO: 3325, and a LC CDR3 comprising the sequence of SEQ ID NO: 3386;
(vii) a VH comprising a HC CDR1 comprising the sequence of SEQ ID NO: 3391, a HC CDR2 comprising the sequence of SEQ ID NO: 3392, a HC CDR3 comprising the sequence of SEQ ID NO: 3393, and/or a VL comprising a LC CDR1 comprising the sequence of SEQ ID NO: 3394, a LC CDR2 comprising the sequence of SEQ ID NO: 3395, and a LC CDR3 comprising the sequence of SEQ ID NO: 3396;
(viii) a VH comprising a HC CDR1 comprising the sequence of SEQ ID NO: 3401, a HC CDR2 comprising the sequence of SEQ ID NO: 3402, a HC CDR3 comprising the sequence of SEQ ID NO: 3403, and/or a VL comprising a LC CDR1 comprising the sequence of SEQ ID NO: 3404, a LC CDR2 comprising the sequence of SEQ ID NO: 3405, and a LC CDR3 comprising the sequence of SEQ ID NO: 3406;
(ix) a VH comprising a HC CDR1 comprising the sequence of SEQ ID NO: 3411, a HC CDR2 comprising the sequence of SEQ ID NO: 3412, a HC CDR3 comprising the sequence of SEQ ID NO: 3413, and/or a VL comprising a LC CDR1 comprising the sequence of SEQ ID NO: 3414, a LC CDR2 comprising the sequence of SEQ ID NO: 3415, and a LC CDR3 comprising the sequence of SEQ ID NO: 3416.
(x) a VH comprising a HC CDR1 comprising the sequence of SEQ ID NO: 3351, SEQ ID NO: 3421, or SEQ ID NO: 3702, a HC CDR2 comprising the sequence of SEQ ID NO: 3352, a HC CDR3 comprising the sequence of SEQ ID NO: 3353, and/or a VL comprising a LC CDR1 comprising the sequence of SEQ ID NO: 3354, a LC CDR2 comprising the sequence of SEQ ID NO: 3355, and a LC CDR3 comprising the sequence of SEQ ID NO: 3356; or
(xi) a VH comprising a HC CDR1 comprising the sequence of SEQ ID NO: 3351, SEQ ID NO: 3421, or SEQ ID NO: 3702, a HC CDR2 comprising the sequence of SEQ ID NO: 3352, a HC CDR3 comprising the sequence of SEQ ID NO: 3353, and/or a VL comprising a LC CDR1 comprising the sequence of SEQ ID NO: 3354, a LC CDR2 comprising the sequence of SEQ ID NO: 3443, and a LC CDR3 comprising the sequence of SEQ ID NO: 3356.

In some embodiments, the second binder comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, or 99.9% sequence identity to any one sequence selected from the group consisting of: SEQ ID NO: 3445, SEQ ID NO: 3448, SEQ ID NO: 3450, SEQ ID NO: 1346, SEQ ID NO: 1196, SEQ ID NO: 3455, SEQ ID NO: 3457, SEQ ID NO: 3463, SEQ ID NO: 3465, and SEQ ID NO: 3470.

In some embodiments, the second binder comprises a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to any one sequence selected from the group consisting of: SEQ ID NO: 3446, SEQ ID NO: 3449, SEQ ID NO: 3451, SEQ ID NO: 1349, SEQ ID NO:1195, SEQ ID NO: 3456, SEQ ID NO: 3458, SEQ ID NO: 3464, SEQ ID NO: 3466, SEQ ID NO: 3471, SEQ ID NO: 3475, and SEQ ID NO: 3476.

In some embodiments, the second binder comprises:
(i) a VH comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to any one sequence selected from the group consisting of: SEQ ID NO: 3445, SEQ ID NO: 3448, SEQ ID NO: 3450, SEQ ID NO: 1346, SEQ ID NO: 1196, SEQ ID NO: 3455, SEQ ID NO: 3457, SEQ ID NO: 3463, SEQ ID NO: 3465, and SEQ ID NO: 3470; and/or
(ii) a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to any one sequence selected from the group consisting of: SEQ ID NO: 3446, SEQ ID NO: 3449, SEQ ID NO: 3451, SEQ ID NO: 1349, SEQ ID NO:1195, SEQ ID NO: 3456, SEQ ID NO: 3458, SEQ ID NO: 3464, SEQ ID NO: 3466, SEQ ID NO: 3471, SEQ ID NO: 3475, and SEQ ID NO: 3476.

In some embodiments, the second binder comprises:
(i) a VH comprising any one sequence selected from the group consisting of: SEQ ID NO: 3445, SEQ ID NO: 3448, SEQ ID NO: 3450, SEQ ID NO: 1346, SEQ ID NO: 1196, SEQ ID NO: 3455, SEQ ID NO: 3457, SEQ ID NO: 3463, SEQ ID NO: 3465, and SEQ ID NO: 3470; and/or
(ii) a VL comprising any one sequence selected from the group consisting of: SEQ ID NO: 3446, SEQ ID NO: 3449, SEQ ID NO: 3451, SEQ ID NO: 1349, SEQ ID NO:1195, SEQ ID NO: 3456, SEQ ID NO: 3458, SEQ ID NO: 3464, SEQ ID NO: 3466, SEQ ID NO: 3471, SEQ ID NO: 3475, and SEQ ID NO: 3476.

In some embodiments, the second binder comprises:
(i) a VH comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3445, and/or a VL comprising a sequence having 30%, 35%, 40%, 45%, 50%, 55%, 60%, 65%, 70%, 75%, 80%, 85%, 90%, 95%, %%, 97%, 98%, 99%, 99.5%, 99.9%, or 100% sequence identity to the sequence of SEQ ID NO: 3446;
(ii) a VH comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3448, and/or a VL comprising a sequence having 30%, 35%, 40%, 45%, 5%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3449;
(iii) a VH comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3450, and/or a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3451;
(iv) a VH comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 1346, and/or a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 1349;
(v) a VH comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 1196, and/or a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 1195;
(vi) a VH comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3455, and/or a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3456;
(vii) a VH comprising a sequence having 30%, 35%, 40%, 45%, 50%, 55%, 60%/0, 65%, 70%, 75%, 80%, 85%, 90%, 95%, 96%, 97%, 98%, 99%, 99.5%, 99.9%, or 100% sequence identity to the sequence of SEQ ID NO: 3457, and/or a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3458;
(viii) a VH comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3463, and/or a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3464;
(ix) a VH comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3465, and/or a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3466;
(x) a VH comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3470, and/or a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3471;
(xi) a VH comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 1346, and/or a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3475; or
(xii) a VH comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 1346, and/or a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3476.

In some embodiments, the second binder comprises:
(i) a VH comprising the sequence of SEQ ID NO: 3445, and/or a VL comprising the sequence of SEQ ID NO: 3446;
(ii) a VH comprising the sequence of SEQ ID NO: 3448, and/or a VL comprising the sequence of SEQ ID NO: 3449;
(iii) a VH comprising the sequence of SEQ ID NO: 3450, and/or a VL comprising the sequence of SEQ ID NO: 3451;
(iv) a VH comprising the sequence of SEQ ID NO: 1346, and/or a VL comprising the sequence of SEQ ID NO: 1349;
(v) a VH comprising the sequence of SEQ ID NO: 1196, and/or a VL comprising the sequence of SEQ ID NO: 1195;
(vi) a VH comprising the sequence of SEQ ID NO: 3455, and/or a VL comprising the sequence of SEQ ID NO: 3456;
(vii) a VH comprising the sequence of SEQ ID NO: 3457, and/or a VL comprising the sequence of SEQ ID NO: 3458;
(viii) a VH comprising the sequence of SEQ ID NO: 3463, and/or a VL comprising the sequence of SEQ ID NO: 3464;
(ix) a VH comprising the sequence of SEQ ID NO: 3465, and/or a VL comprising the sequence of SEQ ID NO: 3466;
(x) a VH comprising the sequence of SEQ ID NO: 3470, and/or a VL comprising the sequence of SEQ ID NO: 3471;
(xi) a VH comprising the sequence of SEQ ID NO: 1346, and/or a VL comprising the sequence of SEQ ID NO: 3475; or
(xii) a VH comprising the sequence of SEQ ID NO: 1346, and/or a VL comprising the sequence of SEQ ID NO: 3476.

In some embodiments, the cytokine molecule comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 2170.

In some embodiments, the IL15Ralpha dimerizing domain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3472.

In some embodiments, the cytokine molecule comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3474.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3445, wherein the VH is linked to a heavy chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3649, wherein the heavy chain constant region comprises the first Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3447,
(ii) the second polypeptide chain comprises a cytokine molecule, wherein the cytokine molecule is linked to the second Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3648 via a linker comprising the sequence of SEQ ID NO: 3308, wherein the cytokine molecule comprises IL-2 or functional variant thereof comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 2270, and
(iii) the third polypeptide chain comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3446, wherein the VL is linked to a light chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3448, wherein the VH is linked to a heavy chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3649, wherein the heavy chain constant region comprises the first Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3447,
(ii) the second polypeptide chain comprises a cytokine molecule, wherein the cytokine molecule is linked to the second Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3648 via a linker comprising the sequence of SEQ ID NO: 3308, wherein the cytokine molecule comprises IL-2 or functional variant thereof comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 2270, and
(iii) the third polypeptide chain comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3449, wherein the VL is linked to a light chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3450, wherein the VH is linked to a heavy chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3649, wherein the heavy chain constant region comprises the first Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3447,
(ii) the second polypeptide chain comprises a cytokine molecule, wherein the cytokine molecule is linked to the second Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3648 via a linker comprising the sequence of SEQ ID NO: 3308, wherein the cytokine molecule comprises IL-2 or functional variant thereof comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 2270, and
(iii) the third polypeptide chain comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3451, wherein the VL is linked to a light chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3450, wherein the VH is linked to a heavy chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3649, wherein the heavy chain constant region comprises the first Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3447,
(ii) the second polypeptide chain comprises a cytokine molecule, wherein the cytokine molecule is linked to the second Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3454 or the sequence of SEQ ID NO: 3501 via a linker comprising the sequence of SEQ ID NO: 3308, wherein the cytokine molecule comprises IL-2 or functional variant thereof comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 2270, and
(iii) the third polypeptide chain comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3451, wherein the VL is linked to a light chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 1346, wherein the VH is linked to a heavy chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3452 or the sequence of SEQ ID NO: 3504, wherein the heavy chain constant region comprises the first Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3453 or the sequence of SEQ ID NO: 3505,
(ii) the second polypeptide chain comprises a cytokine molecule, wherein the cytokine molecule is linked to the second Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3454 or the sequence of SEQ ID NO: 3501 via a linker comprising the sequence of SEQ ID NO: 3308, wherein the cytokine molecule comprises IL-2 or functional variant thereof comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 2270, and
(iii) the third polypeptide chain comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 1349, wherein the VL is linked to a light chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 1196, wherein the VH is linked to a heavy chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3649, wherein the heavy chain constant region comprises the first Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3447,
(ii) the second polypeptide chain comprises a cytokine molecule, wherein the cytokine molecule is linked to the second Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3648 via a linker comprising the sequence of SEQ ID NO: 3308, wherein the cytokine molecule comprises IL-2 or functional variant thereof comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 2270, and
(iii) the third polypeptide chain comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 1195, wherein the VL is linked to a light chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3455, wherein the VH is linked to a heavy chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3649, wherein the heavy chain constant region comprises the first Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3447,
(ii) the second polypeptide chain comprises a cytokine molecule, wherein the cytokine molecule is linked to the second Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3648 via a linker comprising the sequence of SEQ ID NO: 3308, wherein the cytokine molecule comprises IL-2 or functional variant thereof comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 2270, and
(iii) the third polypeptide chain comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3456, wherein the VL is linked to a light chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3457, wherein the VH is linked to a heavy chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3459, wherein the heavy chain constant region comprises the first Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3460,
(ii) the second polypeptide chain comprises a cytokine molecule, wherein the cytokine molecule is linked to the second Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3462 via a linker comprising the sequence of SEQ ID NO: 3308, wherein the cytokine molecule comprises IL-2 or functional variant thereof comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 2191, and
(iii) the third polypeptide chain comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3458, wherein the VL is linked to a light chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3461.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3463, wherein the VH is linked to a heavy chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3459, wherein the heavy chain constant region comprises the first Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3460,
(ii) the second polypeptide chain comprises a cytokine molecule, wherein the cytokine molecule is linked to the second Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3462 via a linker comprising the sequence of SEQ ID NO: 3308, wherein the cytokine molecule comprises IL-2 or functional variant thereof comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 2191, and
(iii) the third polypeptide chain comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3464, wherein the VL is linked to a light chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3461.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3465, wherein the VH is linked to a heavy chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3467, wherein the heavy chain constant region comprises the first Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3468,
(ii) the second polypeptide chain comprises a cytokine molecule, wherein the cytokine molecule is linked to the second Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3469 via a linker comprising the sequence of SEQ ID NO: 3308, wherein the cytokine molecule comprises IL-2 or functional variant thereof comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 2191, and
(iii) the third polypeptide chain comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3466, wherein the VL is linked to a light chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3461.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3470, wherein the VH is linked to a heavy chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3649, wherein the heavy chain constant region comprises the first Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3447,
(ii) the second polypeptide chain comprises a cytokine molecule, wherein the cytokine molecule is linked to the second Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3648 via a linker comprising the sequence of SEQ ID NO: 3308, wherein the cytokine molecule comprises IL-2 or functional variant thereof comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 2270, and
(iii) the third polypeptide chain comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3471, wherein the VL is linked to a light chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 1346, wherein the VH is linked to a heavy chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3452 or the sequence of SEQ ID NO: 3504, wherein the heavy chain constant region comprises the first Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3453 or the sequence of SEQ ID NO: 3505,
(ii) the second polypeptide chain comprises a cytokine molecule, wherein the cytokine molecule is linked to the second Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3648 via a linker comprising the sequence of SEQ ID NO: 3308, wherein the cytokine molecule comprises IL-15 or functional variant thereof comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 2170, wherein the IL-15 or functional variant thereof is covalently linked to an IL15Ralpha dimerizing domain comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of the SEQ ID NO: 3472 via a linker comprising the sequence of SEQ ID NO: 3473, and
(iii) the third polypeptide chain comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 1349, wherein the VL is linked to a light chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 1346, wherein the VH is linked to a heavy chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3452 or the sequence of SEQ ID NO: 3504, wherein the heavy chain constant region comprises the first Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3453 or the sequence of SEQ ID NO: 3505,
(ii) the second polypeptide chain comprises a cytokine molecule, wherein the cytokine molecule is linked to the second Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3648 via a linker comprising the sequence of SEQ ID NO: 3308, wherein the cytokine molecule comprises IL-15 or functional variant thereof comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 2170, wherein the IL-15 or functional variant thereof is covalently linked to an IL15Ralpha dimerizing domain comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of the SEQ ID NO: 3472 via a linker comprising the sequence of SEQ ID NO: 3473, and
(iii) the third polypeptide chain comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3475, wherein the VL is linked to a light chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3644.

In some embodiments, the first agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 1346, wherein the VH is linked to a heavy chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3452 or the sequence of SEQ ID NO: 3504, wherein the heavy chain constant region comprises the first Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3453 or the sequence of SEQ ID NO: 3505,
(ii) the second polypeptide chain comprises a cytokine molecule, wherein the cytokine molecule is linked to the second Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3648 via a linker comprising the sequence of SEQ ID NO: 3308, wherein the cytokine molecule comprises IL-15 or functional variant thereof comprising a sequence having at least 30%, 35%, 40%, 45%, 50%, 55%, 60%, 65%, 70%, 75%, 80%, 85%, 90%, 95%, 96%, 97%, 98%, 99%. 99.5%, 99.9%, or 100% sequence identity to the sequence of SEQ ID NO: 2170, wherein the IL-15 or functional variant thereof is covalently linked to an IL15Ralpha dimerizing domain comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of the SEQ ID NO: 3472 via a linker comprising the sequence of SEQ ID NO: 3473, and
(iii) the third polypeptide chain comprises a VL comprising a sequence having at least 30%, 35%, 40%, 45%, 50%, 55%, 60%, 65%, 70%, 75%, 80%, 85%, 90%, 95%, %%, 97%, 98%, 99%, 99.5%, 99.9%, or 100% sequence identity to the sequence of SEQ ID NO: 3476, wherein the VL is linked to a light chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3445, wherein the VH is linked to a heavy chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3649, wherein the heavy chain constant region comprises the first Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3447,
(ii) the second polypeptide chain comprises a cytokine molecule, wherein the cytokine molecule is linked to the second Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3648 via a linker comprising the sequence of SEQ ID NO: 3308, wherein the cytokine molecule comprises IL-2 or functional variant thereof comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 2270, and
(iii) the third polypeptide chain comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3446, wherein the VL is linked to a light chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3448, wherein the VH is linked to a heavy chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3649, wherein the heavy chain constant region comprises the first Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3447,
(ii) the second polypeptide chain comprises a cytokine molecule, wherein the cytokine molecule is linked to the second Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3648 via a linker comprising the sequence of SEQ ID NO: 3308, wherein the cytokine molecule comprises IL-2 or functional variant thereof comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 2270, and
(iii) the third polypeptide chain comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3449, wherein the VL is linked to a light chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3450, wherein the VH is linked to a heavy chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3649, wherein the heavy chain constant region comprises the first Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3447,
(ii) the second polypeptide chain comprises a cytokine molecule, wherein the cytokine molecule is linked to the second Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3648 via a linker comprising the sequence of SEQ ID NO: 3308, wherein the cytokine molecule comprises IL-2 or functional variant thereof comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 2270, and
(iii) the third polypeptide chain comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3451, wherein the VL is linked to a light chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9% or 100% sequence identity to the sequence of SEQ ID NO: 3450, wherein the VH is linked to a heavy chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3649, wherein the heavy chain constant region comprises the first Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3447,
(ii) the second polypeptide chain comprises a cytokine molecule, wherein the cytokine molecule is linked to the second Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3454 or the sequence of SEQ ID NO: 3501 via a linker comprising the sequence of SEQ ID NO: 3308, wherein the cytokine molecule comprises IL-2 or functional variant thereof comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 2270, and
(iii) the third polypeptide chain comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3451, wherein the VL is linked to a light chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 1346, wherein the VH is linked to a heavy chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3452 or the sequence of SEQ ID NO: 3504, wherein the heavy chain constant region comprises the first Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3453 or the sequence of SEQ ID NO: 3505,
(ii) the second polypeptide chain comprises a cytokine molecule, wherein the cytokine molecule is linked to the second Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3454 or the sequence of SEQ ID NO: 3501 via a linker comprising the sequence of SEQ ID NO: 3308, wherein the cytokine molecule comprises IL-2 or functional variant thereof comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 2270, and
(iii) the third polypeptide chain comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 1349, wherein the VL is linked to a light chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 1196, wherein the VH is linked to a heavy chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3649, wherein the heavy chain constant region comprises the first Fc region comprising a sequence having at least 30%, 35%, 40%, 45%, 50%, 55%, 60%, 65%, 70%, 75%, 80%, 85%, 90%, 95%, 96%, 97%, 98% 99%, 99.5% 99.9%, or 100% sequence identity to the sequence of SEQ ID NO: 3447,
(ii) the second polypeptide chain comprises a cytokine molecule, wherein the cytokine molecule is linked to the second Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3648 via a linker comprising the sequence of SEQ ID NO: 3308, wherein the cytokine molecule comprises IL-2 or functional variant thereof comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 2270, and
(iii) the third polypeptide chain comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 1195, wherein the VL is linked to a light chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3455, wherein the VH is linked to a heavy chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3649, wherein the heavy chain constant region comprises the first Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3447,
(ii) the second polypeptide chain comprises a cytokine molecule, wherein the cytokine molecule is linked to the second Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3648 via a linker comprising the sequence of SEQ ID NO: 3308, wherein the cytokine molecule comprises IL-2 or functional variant thereof comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 2270, and
(iii) the third polypeptide chain comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3456, wherein the VL is linked to a light chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3457, wherein the VH is linked to a heavy chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3459, wherein the heavy chain constant region comprises the first Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3460,
(ii) the second polypeptide chain comprises a cytokine molecule, wherein the cytokine molecule is linked to the second Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3462 via a linker comprising the sequence of SEQ ID NO: 3308, wherein the cytokine molecule comprises IL-2 or functional variant thereof comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 2191, and
(iii) the third polypeptide chain comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3458, wherein the VL is linked to a light chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3461.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3463, wherein the VH is linked to a heavy chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3459, wherein the heavy chain constant region comprises the first Fc region comprising a sequence having at least 30%, 35%, 40%, 45%, 50%, 55%, 60%, 65%, 70%, 75%, 80%, 85%, 90%, 95%, 96%, 97%, 98% 99%, 99.5% 99.9%, or 100% sequence identity to the sequence of SEQ ID NO: 3460,
(ii) the second polypeptide chain comprises a cytokine molecule, wherein the cytokine molecule is linked to the second Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3462 via a linker comprising the sequence of SEQ ID NO: 3308, wherein the cytokine molecule comprises IL-2 or functional variant thereof comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 2191, and
(iii) the third polypeptide chain comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3464, wherein the VL is linked to a light chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3461.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3465, wherein the VH is linked to a heavy chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3467, wherein the heavy chain constant region comprises the first Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3468,
(ii) the second polypeptide chain comprises a cytokine molecule, wherein the cytokine molecule is linked to the second Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3469 via a linker comprising the sequence of SEQ ID NO: 3308, wherein the cytokine molecule comprises IL-2 or functional variant thereof comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 2191, and
(iii) the third polypeptide chain comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3466, wherein the VL is linked to a light chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3461.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3470, wherein the VH is linked to a heavy chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3649, wherein the heavy chain constant region comprises the first Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3447,
(ii) the second polypeptide chain comprises a cytokine molecule, wherein the cytokine molecule is linked to the second Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3648 via a linker comprising the sequence of SEQ ID NO: 3308, wherein the cytokine molecule comprises IL-2 or functional variant thereof comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 2270, and
(iii) the third polypeptide chain comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3471, wherein the VL is linked to a light chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 1346, wherein the VH is linked to a heavy chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3452 or the sequence of SEQ ID NO: 3504, wherein the heavy chain constant region comprises the first Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3453 or the sequence of SEQ ID NO: 3505,
(ii) the second polypeptide chain comprises a cytokine molecule, wherein the cytokine molecule is linked to the second Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3648 via a linker comprising the sequence of SEQ ID NO: 3308, wherein the cytokine molecule comprises IL-15 or functional variant thereof comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 2170, wherein the IL-15 or functional variant thereof is covalently linked to an IL15Ralpha dimerizing domain comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of the SEQ ID NO: 3472 via a linker comprising the sequence of SEQ ID NO: 3473, and
(iii) the third polypeptide chain comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 1349, wherein the VL is linked to a light chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 1346, wherein the VH is linked to a heavy chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3452 or the sequence of SEQ ID NO: 3504, wherein the heavy chain constant region comprises the first Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3453 or the sequence of SEQ ID NO: 3505,
(ii) the second polypeptide chain comprises a cytokine molecule, wherein the cytokine molecule is linked to the second Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3648 via a linker comprising the sequence of SEQ ID NO: 3308, wherein the cytokine molecule comprises IL-15 or functional variant thereof comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 2170, wherein the IL-15 or functional variant thereof is covalently linked to an IL15Ralpha dimerizing domain comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of the SEQ ID NO: 3472 via a linker comprising the sequence of SEQ ID NO: 3473, and
(iii) the third polypeptide chain comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3475, wherein the VL is linked to a light chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 1346, wherein the VH is linked to a heavy chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3452 or the sequence of SEQ ID NO: 3504, wherein the heavy chain constant region comprises the first Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3453 or the sequence of SEQ ID NO: 3505,
(ii) the second polypeptide chain comprises a cytokine molecule, wherein the cytokine molecule is linked to the second Fc region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3648 via a linker comprising the sequence of SEQ ID NO: 3308, wherein the cytokine molecule comprises IL-15 or functional variant thereof comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 2170, wherein the IL-15 or functional variant thereof is covalently linked to an IL15Ralpha dimerizing domain comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of the SEQ ID NO: 3472 via a linker comprising the sequence of SEQ ID NO: 3473, and
(iii) the third polypeptide chain comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3476, wherein the VL is linked to a light chain constant region comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3644.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3338;
(ii) the second polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3340 or the sequence of SEQ ID NO: 3503; and
(iii) the third polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3339.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3318;
(ii) the second polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3340 or the sequence of SEQ ID NO: 3503; and
(iii) the third polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3319.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3328;
(ii) the second polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3340 or the sequence of SEQ ID NO: 3503; and
(iii) the third polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3329.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3328;
(ii) the second polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3350 or the sequence of SEQ ID NO: 3500; and
(iii) the third polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3329.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3348 or the sequence of SEQ ID NO: 3502;
(ii) the second polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3340 or the sequence of SEQ ID NO: 3503; and
(iii) the third polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3349.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3358;
(ii) the second polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3340 or the sequence of SEQ ID NO: 3503; and
(iii) the third polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3359.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3368;
(ii) the second polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3340 or the sequence of SEQ ID NO: 3503; and
(iii) the third polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3369.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3378;
(ii) the second polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3380; and
(iii) the third polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3379.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3388;
(ii) the second polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3390, and
(iii) the third polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3389.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3398;
(ii) the second polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3400; and
(iii) the third polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3399.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3408;
(ii) the second polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3340 or the sequence of SEQ ID NO: 3503; and
(iii) the third polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3409.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3348 or the sequence of SEQ ID NO: 3502;
(ii) the second polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3420, and
(iii) the third polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3349.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3348 or the sequence of SEQ ID NO: 3502;
(ii) the second polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3420; and
(iii) the third polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3428.

In some embodiments, the second agent comprises a first polypeptide chain, a second polypeptide chain, and a third polypeptide chain, wherein the first polypeptide chain, the second polypeptide chain, and the third polypeptide chain are non-contiguous, and wherein:
(i) the first polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3348 or the sequence of SEQ ID NO: 3502;
(ii) the second polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3420, and
(iii) the third polypeptide chain comprises a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to the sequence of SEQ ID NO: 3437.

In some embodiments, the first binder comprises a VH comprising a HC CDR3 comprising any one of the HC CDR3 sequences listed in Tables 1, 2, 10A, 10B, 10C, 11-13, and 15-23.

In some embodiments, the first binder comprises a VH comprising a HC CDR1 comprising any one of the HC CDR1 sequences listed in Tables 1, 2, 10A, 10B, 10C, 11-13, and 15-23.

In some embodiments, the first binder comprises a VH comprising a HC CDR2 comprising any one of the HC CDR2 sequences listed in Tables 1, 2, 10A, 10B, 10C, 11-13, and 15-23.

In some embodiments, the first binder comprises a VH comprising a HC CDR1, a HC CDR2, and a HC CDR3 comprising any one of the HC CDR1, the HC CDR2, and the HC CDR3 sequences listed in Tables 1, 2, 10A, 10B, 10C, 11-13, and 15-23.

In some embodiments, the first binder comprises a VL comprising a LC CDR3 comprising any one of the LC CDR3 sequences listed in Tables 1, 2, 10A, 10B, 10C, 11-13, and 15-23.

In some embodiments, the first binder comprises a VL comprising a LC CDR1 comprising any one of the LC CDR1 sequences listed in Tables 1, 2, 10A, 10B, 10C, 11-13, and 15-23.

In some embodiments, the first binder comprises a VL comprising a LC CDR2 comprising any one of the LC CDR2 sequences listed in Tables 1, 2, 10A, 10B, 10C, 11-13, and 15-23.

In some embodiments, the first binder comprises a VL comprising a LC CDR1, a LC CDR2, and a LC CDR3 comprising any one of the LC CDR1, the LC CDR2, and the LC CDR3 sequences listed in Tables 1, 2, 10A, 10B, 10C, 11-13, and 15-23.

In some embodiments, the first binder comprises a VH comprising a HC CDR1, a HC CDR2, and a HC CDR3 comprising any one of the HC CDR1, the HC CDR2, and the HC CDR3 sequences listed in Tables 1, 2, 10A, 10B, 10C, 11-13, and 15-23, and/or a VL comprising a LC CDR1, a LC CDR2, and a LC CDR3 comprising any one of the LC CDR1, the LC CDR2, and the LC CDR3 sequences listed in Tables 1, 2, 10A, 10B, 10C, 11-13, and 15-23.

In some embodiments, the first binder comprises a VH comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to any one of the VH sequence sequences listed in Tables 1, 2, 10A, 10B, 10C, 11-13, and 15-23.

In some embodiments, the first binder comprises a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to any one of the VL sequence sequences listed in Tables 1, 2, 10A, 10B, 10C, 11-13, and 15-23.

In some embodiments, the first binder comprises a VH comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to any one of the VH sequence sequences listed in Tables 1, 2, 10A, 10B, 10C, 11-13, and 15-23, and/or a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to any one of the VL sequence sequences listed in Tables 1, 2, 10A, 10B, 10C, 11-13, and 15-23.

In some embodiments, the second binder comprises a VH comprising a HC CDR3 comprising any one of the HC CDR3 sequences listed in Tables 1, 2, 10A, 10B, 10C, 11-13, and 15-23.

In some embodiments, the second binder comprises a VH comprising a HC CDR1 comprising any one of the HC CDR1 sequences listed in Tables 1, 2, 10A, 10B, 10C, 11-13, and 15-23.

In some embodiments, the second binder comprises a VH comprising a HC CDR2 comprising any one of the HC CDR2 sequences listed in Tables 1, 2, 10A, 10B, 10C, 11-13, and 15-23.

In some embodiments, the second binder comprises a VH comprising a HC CDR1, a HC CDR2, and a HC CDR3 comprising any one of the HC CDR1, the HC CDR2, and the HC CDR3 sequences listed in Tables 1, 2, 10A, 10B, 10C, 11-13, and 15-23.

In some embodiments, the second binder comprises a VL comprising a LC CDR3 comprising any one of the LC CDR3 sequences listed in Tables 1, 2, 10A, 10B, 10C, 11-13, and 15-23.

In some embodiments, the second binder comprises a VL comprising a LC CDR1 comprising any one of the LC CDR1 sequences listed in Tables 1, 2, 10A, 10B, 10C, 11-13, and 15-23.

In some embodiments, the second binder comprises a VL comprising a LC CDR2 comprising any one of the LC CDR2 sequences listed in Tables 1, 2, 10A, 10B, 10C, 11-13, and 15-23.

In some embodiments, the second binder comprises a VL comprising a LC CDR1, a LC CDR2, and a LC CDR3 comprising any one of the LC CDR1, the LC CDR2, and the LC CDR3 sequences listed in Tables 1, 2, 10A, 10B, 10C, 11-13, and 15-23.

In some embodiments, the second binder comprises a VH comprising a HC CDR1, a HC CDR2, and a HC CDR3 comprising any one of the HC CDR1, the HC CDR2, and the HC CDR3 sequences listed in Tables 1, 2, 10A, 10B, 10C, 11-13, and 15-23, and/or a VL comprising a LC CDR1, a LC CDR2, and a LC CDR3 comprising any one of the LC CDR1, the LC CDR2, and the LC CDR3 sequences listed in Tables 1, 2, 10A, 10B, 10C, 11-13, and 15-23.

In some embodiments, the second binder comprises a VH comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to any one of the VH sequence sequences listed in Tables 1, 2, 10A, 10B, 10C, 11-13, and 15-23.

In some embodiments, the second binder comprises a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to any one of the VL sequence sequences listed in Tables 1, 2, 10A, 10B, 10C, 11-13, and 15-23.

In some embodiments, the second binder comprises a VH comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to any one of the VH sequence sequences listed in Tables 1, 2, 10A, 10B, 10C, 11-13, and 15-23, and/or a VL comprising a sequence having 30%, 35%, 40%, 45%, 50%, 55%, 60%, 65%, 70%, 75%, 80%, 85%, 90%, 95%, 96%, 97%, 98%, 99%, 99.5%, 99.9%, or 100% sequence identity to any one of the VL sequence sequences listed in Tables 1, 2, 10A, 10B, 10C, 11-13, and 15-23.

In some embodiments, the first binder or the first agent comprises one or more heavy chain constant regions, wherein the one or more heavy chain constant regions comprises a sequence having at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or 100% sequence identity any one of the heavy chain constant region sequences listed in Tables 1, 2, 10A, 10B, 10C, 11-14, and 15-23.

In some embodiments, the first binder or the first agent comprises a first portion of the dimerization module comprising a first immunoglobulin constant region (Fc region) and a second portion of the dimerization module comprising a second Fc region, wherein the first Fc region, the second Fc region, or a combination thereof comprises a sequence having at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or 100% sequence identity to any one of the Fc region sequences listed in Tables 1, 2, 10A, 10B, 10C, 11-14, and 15-23.

In some embodiments, the first binder or the first agent comprises a first portion of the dimerization module comprising a first immunoglobulin constant region (Fc region) and a second portion of the dimerization module comprising a second Fc region, wherein the first Fc region, the second Fc region, or a combination thereof comprises one or more mutations listed in Table 14.

In some embodiments, the first binder or the first agent comprises a kappa light chain constant region or functional fragment thereof, a lambda light chain constant region or functional fragment thereof, or a combination thereof, wherein the kappa light chain constant region or the lambda light chain constant region comprises a sequence having at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or 100% sequence identity to any one of the light chain constant region sequences listed in Tables 1, 2, 10A, 10B, 10C, 11-14, and 15-23.

In some embodiments, the first agent comprises at least one cytokine molecule, wherein the at least one cytokine molecule comprises a sequence having at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or 100% sequence identity to any one of the cytokine molecule sequences listed in Tables 1, 2, 10A, 10B, 10C, 11-13, and 15-23.

In some embodiments, the first agent comprises at least one cytokine molecule, wherein the at least one cytokine molecule further comprises a cytokine receptor or a functional fragment or variant thereof, wherein cytokine receptor or a functional fragment or variant thereof comprises a sequence having at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or 100% sequence identity to any one of the cytokine receptor sequences listed in Tables 1, 2, 10A, 10B, 10C, 11-13, and 15-23.

In some embodiments, the first agent comprises:
(a) a first polypeptide comprises a sequence having at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or 100% sequence identity to any one of the polypeptide sequences listed in Tables 1, 2, 10A, 10B, 10C, 11-13, and 15-23;
(b) the second polypeptide comprises a sequence having at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or 100% sequence identity to any one of the polypeptide sequences listed in Tables 1, 2, 10A, 10B, 10C, 11-13, and 15-23; and
(c) the third polypeptide comprises a sequence having at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or 100% sequence identity to any one of the polypeptide sequences listed in Tables 1, 2, 10A, 10B, 10C, 11-13, and 15-23.

In some embodiments, the second binder or the second agent comprises one or more heavy chain constant regions, wherein the one or more heavy chain constant regions comprises a sequence having at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or 100% sequence identity any one of the heavy chain constant region sequences listed in Tables 1, 2, 10A, 10B, 10C, 11-14, and 15-23.

In some embodiments, the second binder or the second agent comprises a first portion of the dimerization module comprising a first immunoglobulin constant region (Fc region) and a second portion of the dimerization module comprising a second Fc region, wherein the first Fc region, the second Fc region, or a combination thereof comprises a sequence having at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or 100% sequence identity to any one of the Fc region sequences listed in Tables 1, 2, 10A, 10B, 10C, 11-14, and 15-23.

In some embodiments, the second binder or the second agent comprises a first portion of the dimerization module comprising a first immunoglobulin constant region (Fc region) and a second portion of the dimerization module comprising a second Fc region, wherein the first Fc region, the second Fc region, or a combination thereof comprises one or more mutations listed in Table 14.

In some embodiments, the second binder or the second agent comprises a kappa light chain constant region or functional fragment thereof, a lambda light chain constant region or functional fragment thereof, or a combination thereof, wherein the kappa light chain constant region or the lambda light chain constant region comprises a sequence having at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or 100% sequence identity to any one of the light chain constant region sequences listed in Tables 1, 2, 10A, 10B, 10C, 11-14, and 15-23.

In some embodiments, the second agent comprises at least one cytokine molecule, wherein the at least one cytokine molecule comprises a sequence having at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or 100% sequence identity to any one of the cytokine molecule sequences listed in Tables 1, 2, 10A, 10B, 10C, 11-13, and 15-23.

In some embodiments, the second agent comprises at least one cytokine molecule, wherein the at least one cytokine molecule further comprises a cytokine receptor or a functional fragment or variant thereof, wherein cytokine receptor or a functional fragment or variant thereof comprises a sequence having at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or 100% sequence identity to any one of the cytokine receptor sequences listed in Tables 1, 2, 10A, 10B, 10C, 11-13, and 15-23.

In some embodiments, the second agent comprises:
(a) a first polypeptide comprises a sequence having at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or 100% sequence identity to any one of the polypeptide sequences listed in Tables 1, 2, 10A, 10B, 10C, 11-13, and 15-23;
(b) the second polypeptide comprises a sequence having at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or 100% sequence identity to any one of the polypeptide sequences listed in Tables 1, 2, 10A, 10B, 10C, 11-13, and 15-23; and
(c) the third polypeptide comprises a sequence having at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or 100% sequence identity to any one of the polypeptide sequences listed in Tables 1, 2, 10A, 10B, 10C, 11-13, and 15-23.

TABLE 1
Amino acid and nucleotide sequences for murine, chimeric and humanized antibody molecules
which bind to TCRVB 6, e.g., TCRVB 6-5. The antibody molecules include murine mAb
Antibody A, and humanized mAb Antibody A-H Clones A-H.1 to A-H.85. The amino acid the
heavy and light chain CDRs, and the amino acid and nucleotide sequences of the heavy
and light chain variable regions, and the heavy and light chains are shown.
Antibody A (murine), also referred to as H131, TCRVB 6-5 binder

| | | |
|---|---|---|
| SEQ ID NO: 3 | HC CDR1 (Combined) | GYSFTTYYIH |
| SEQ ID NO: 4 | HC CDR2 (Combined) | WFFPGSGNIKYNEKFKG |
| SEQ ID NO: 5 | HC CDR3 (Combined) | SYYSYDVLDY |
| SEQ ID NO: 45 | HC CDR1 (Kabat) | TYYIH |
| SEQ ID NO: 46 | HC CDR2 (Kabat) | WFFPGSGNIKYNEKFKG |
| SEQ ID NO: 47 | HC CDR3 (Kabat) | SYYSYDVLDY |
| SEQ ID NO: 48 | HC CDR1 (Chothia) | GYSFTTY |
| SEQ ID NO: 49 | HC CDR2 (Chothia) | FPGSGN |
| SEQ ID NO: 50 | HC CDR3 (Chothia) | SYYSYDVLDY |
| SEQ ID NO: 1 | VH | QVQLQQSGPELVKPGTSVKISCKASGYSFTTYYIHWVKQRPG |
| | | QGLEWIGWFFPGSGNIKYNEKFKGKATLTADTSSSTAYMQLS |
| | | SLTSEESAVYFCAGSYYSYDVLDYWGHGTTLTVSS |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 51 | LC CDR1 (Kabat) | KASQNVGINVV |
| SEQ ID NO: 52 | LC CDR2 (Kabat) | SSSHRYS |
| SEQ ID NO: 53 | LC CDR3 (Kabat) | QQFKSYPLT |
| SEQ ID NO: 54 | LC CDR1 (Chothia) | KASQNVGINVV |
| SEQ ID NO: 55 | LC CDR2 (Chothia) | SSSHRYS |
| SEQ ID NO: 56 | LC CDR3 (Chothia) | QQFKSYPLT |
| SEQ ID NO: 2 | VL | DILMTQSQKFMSTSLGDRVSVSCKASQNVGINVVWHQQKPG |
| | | QSPKALIYSSSHRYSGVPDRFTGSGSGTDFTLTINNVQSEDLA |
| | | EYFCQQFKSYPLTFGAGTKLELK |

Antibody A humanized (A-H antibody), TCRVB 6-5 binder
A-H.1 antibody (also referred to as BHM1709)

| | | |
|---|---|---|
| SEQ ID NO: 3 | HC CDR1 (Combined) | GYSFTTYYIH |
| SEQ ID NO: 4 | HC CDR2 (Combined) | WFFPGSGNIKYNEKFKG |
| SEQ ID NO: 5 | HC CDR3 (Combined) | SYYSYDVLDY |
| SEQ ID NO: 9 | VH | QVQLVQSGAEVKKPGSSVKVSCKASGYSFTTYYIHWVRQAP |
| | | GQGLEWMGWFFPGSGNIKYNEKFKGRVTITADTSTSTAYME |
| | | LSSLRSEDTAVYYCAGSYYSYDVLDYWGQGTTVTVSS |
| SEQ ID NO: 12 | DNA VH | CAGGTGCAGCTGGTTCAGTCTGGCGCCGAAGTGAAGAAAC |
| | | CTGGCTCCTCCGTGAAGGTGTCCTGCAAGGCTTCCGGCTAC |
| | | TCCTTCACCACCTACTACATCCACTGGGTCCGACAGGCCCC |
| | | TGGACAAGGATTGGAATGGATGGGCTGGTTCTTCCCCGGC |
| | | TCCGGCAACATCAAGTACAACGAGAAGTTCAAGGGCCGCG |
| | | TGACCATCACCGCCGACACCTCTACCTCTACCGCCTACATG |
| | | GAACTGTCCAGCCTGAGATCTGAGGACACCGCCGTGTACT |
| | | ACTGCGCCGGCTCCTACTACTCTTACGACGTGCTGGATTAC |
| | | TGGGGCCAGGGCACCACAGTGACAGTGTCCTCT |
| SEQ ID NO: 69 | VH-IgM constant delta | METDTLLLWVLLLWVPGSTGQVQLVQSGAEVKKPGSSVKVS |
| | CDC | CKASGYSFTTYYIHWVRQAPGQGLEWMGWFFPGSGNIKYNE |
| | | KFKGRVTITADTSTSTAYMELSSLRSEDTAVYYCAGSYYSYD |
| | | VLDYWGQGTTVTVSSGSASAPTLFPLVSCENSPSDTSSVAVG |
| | | CLAQDFLPDSITFSWKYKNNSDISSTRGFPSVLRGGKYAATSQ |
| | | VLLPSKDVMQGTDEHVVCKVQHPNGNKEKNVPLPVIAELPP |
| | | KVSVFVPPRDGFFGNPRKSKLICQATGFSPRQIQVSWLREGKQ |
| | | VGSGVTTDQVQAEAKESGPTTYKVTSTLTIKESDWLGQSMFT |
| | | CRVDHRGLTFQQNASSMCVPDQDTAIRVFAIPPSFASIFLTKS |
| | | TKLTCLVTDLTTYDSVTISWTRQNGEAVKTHTNISESHPNATF |
| | | SAVGEASICEDDWNSGERFTCTVTHTDLASSLKQTISRPKGVA |
| | | LHRPDVYLLPPAREQLNLRESATITCLVTGFSPADVFVQWMQ |
| | | RGQPLSPEKYVTSAPMPEPQAPGRYFAHSILTVSEEEWNTGET |
| | | YTCVVAHEALPNRVTERTVDKSTGKPTLYNVSLVMSDTAGT |
| | | CY |
| SEQ ID NO: 70 | VH-IgGA1 | METDTLLLWVLLLWVPGSTGQVQLVQSGAEVKKPGSSVKVS |
| | | CKASGYSFTTYYIHWVRQAPGQGLEWMGWFFPGSGNIKYNE |
| | | KFKGRVTITADTSTSTAYMELSSLRSEDTAVYYCAGSYYSYD |
| | | VLDYWGQGTTVTVSSASPTSPKVFPLSLCSTQPDGNVVIACL |
| | | VQGFFPQEPLSVTWSESGQGVTARNFPPSQDASGDLYTTSSQ |
| | | LTLPATQCLAGKSVTCHVKHYTNPSQDVTVPCPVPSTPPTPSP |
| | | STPPTPSPSCCHPRLSLHRPALEDLLLGSEANLTCTLTGLRDAS |
| | | GVTFTWTPSSGKSAVQGPPERDLCGCYSVSSVLPGCAEPWNH |
| | | GKTFTCTAAYPESKTPLTATLSKSGNTFRPEVHLLPPPSEELAL |
| | | NELVTLTCLARGFSPKDVLVRWLQGSQELPREKYLTWASRQ |
| | | EPSQGTTTFAVTSILRVAAEDWKKGDTFSCMVGHEALPLAFT |
| | | QKTIDRLAGKPTHVNVSVVMAEVDGTCY |
| SEQ ID NO: 71 | VH-IgGA2 | METDTLLLWVLLLWVPGSTGQVQLVQSGAEVKKPGSSVKVS |
| | | CKASGYSFTTYYIHWVRQAPGQGLEWMGWFFPGSGNIKYNE |
| | | KFKGRVTITADTSTSTAYMELSSLRSEDTAVYYCAGSYYSYD |
| | | VLDYWGQGTTVTVSSASPTSPKVFPLSLDSTPQDGNVVVACL |
| | | VQGFFPQEPLSVTWSESGQNVTARNFPPSQDASGDLYTTSSQ |
| | | LTLPATQCPDGKSVTCHVKHYTNSSQDVTVPCRVPPPPPCCH |
| | | PRLSLHRPALEDLLLGSEANLTCTLTGLRDASGATFTWTPSSG |
| | | KSAVQGPPERDLCGCYSVSSVLPGCAQPWNHGETFTCTAAH |
| | | PELKTPLTANITKSGNTFRPEVHLLPPPSEELALNELVTLTCLA |
| | | RGFSPKDVLVRWLQGSQELPREKYLTWASRQEPSQGTTTYA |
| | | VTSILRVAAEDWKKGETFSCMVGHEALPLAFTQKTIDRMAG |
| | | KPTHINVSVVMAEADGTCY |
| SEQ ID NO: | Heavy chain | METDTLLLWVLLLWVPGSTGQVQLVQSGAEVKKPGSSVKVS |
| 3278 | | CKASGYSFTTYYIHWVRQAPGQGLEWMGWFFPGSGNIKYNE |
| | | KFKGRVTITADTSTSTAYMELSSLRSEDTAVYYCAGSYYSYD |
| | | VLDYWGQGTTVTVSSASTKGPSVFPLAPSSKSTSGGTAALGC |
| | | LVKDYFPEPVTVSWNSGALTSGVHTFPAVLQSSGLYSLSSVV |
| | | TVPSSSLGTQTYICNVNHKPSNTKVDKRVEPKSCDKTHTCPP |
| | | CPAPELLGGPSVFLFPPKPKDTLMISRTPEVTCVVVDVSHEDP |
| | | EVKFNWYVDGVEVHNAKTKPREEQYNSTYRVVSVLTVLHQ |
| | | DWLNGKEYKCKVSNKALPAPIEKTISKAKGQPREPQVYTLPP |
| | | SREEMTKNQVSLTCLVKGFYPSDIAVEWESNGQPENNYKTTP |
| | | PVLDSDGSFFLYSKLTVDKSRWQQGNVFSCSVMHEALHNHY |
| | | TQKSLSLSPGK |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 10 | VL | DIQMTQSPSFLSASVGDRVTITCKASQNVGINVVWHQQKPGK |
| | | APKALIYSSSHRYSGVPSRFSGSGSGTEFTLTISSLQPEDFATYF |
| | | CQQFKSYPLTFGQGTKLEIK |
| SEQ ID NO: 13 | DNA VL | GACATCCAGATGACCCAGTCTCCATCCTTCCTGTCCGCCTC |
| | | TGTGGGCGACAGAGTGACCATCACATGCAAGGCCTCTCAG |
| | | AACGTGGGCATCAACGTCGTGTGGCACCAGCAGAAGCCTG |
| | | GCAAGGCTCCTAAGGCTCTGATCTACTCCTCCAGCCACCG |
| | | GTACTCTGGCGTGCCCTCTAGATTTTCCGGCTCTGGCTCTG |
| | | GCACCGAGTTTACCCTGACAATCTCCAGCCTGCAGCCTGA |
| | | GGACTTCGCCACCTACTTTTGCCAGCAGTTCAAGAGCTACC |
| | | CTCTGACCTTTGGCCAGGGCACCAAGCTGGAAATCAAG |
| SEQ ID NO: 72 | VL and kappa constant | METDTLLLWVLLLWVPGSTGDIQMTQSPSFLSASVGDRVTIT |
| | region/light chain | CKASQNVGINVVWHQQKPGKAPKALIYSSSHRYSGVPSRFSG |
| | | SGSGTEFTLTISSLQPEDFATYFCQQFKSYPLTFGQGTKLEIKR |
| | | TVAAPSVFIFPPSDEQLKSGTASVVCLLNNFYPREAKVQWKV |
| | | DNALQSGNSQESVTEQDSKDSTYSLSSTLTLSKADYEKHKVY |
| | | ACEVTHQGLSSPVTKSFNRGEC |

A-H.2 antibody (also referred to as BHM1710)

| | | |
|---|---|---|
| SEQ ID NO: 3 | HC CDR1 (Combined) | GYSFTTYYIH |
| SEQ ID NO: 4 | HC CDR2 (Combined) | WFFPGSGNIKYNEKFKG |
| SEQ ID NO: 5 | HC CDR3 (Combined) | SYYSYDVLDY |
| SEQ ID NO: 9 | VH | QVQLVQSGAEVKKPGSSVKVSCKASGYSFTTYYIHWVRQAP |
| | | GQGLEWMGWFFPGSGNIKYNEKFKGRVTITADTSTSTAYME |
| | | LSSLRSEDTAVYYCAGSYYSYDVLDYWGQGTTVTVSS |
| SEQ ID NO: 12 | DNA VH | CAGGTGCAGCTGGTTCAGTCTGGCGCCGAAGTGAAGAAAC |
| | | CTGGCTCCTCCGTGAAGGTGTCCTGCAAGGCTTCCGGCTAC |
| | | TCCTTCACCACCTACTACATCCACTGGGTCCGACAGGCCCC |
| | | TGGACAAGGATTGGAATGGATGGGCTGGTTCTTCCCCGGC |
| | | TCCGGCAACATCAAGTACAACGAGAAGTTCAAGGGCCGCG |
| | | TGACCATCACCGCCGACACCTCTACCTCTACCGCCTACATG |
| | | GAACTGTCCAGCCTGAGATCTGAGGACACCGCCGTGTACT |
| | | ACTGCGCCGGCTCCTACTACTCTTACGACGTGCTGGATTAC |
| | | TGGGGCCAGGGCACCACAGTGACAGTGTCCTCT |
| SEQ ID NO: | Heavy chain | METDTLLLWVLLLWVPGSTGQVQLVQSGAEVKKPGSSVKVS |
| 3278 | | CKASGYSFTTYYIHWVRQAPGQGLEWMGWFFPGSGNIKYNE |
| | | KFKGRVTITADTSTSTAYMELSSLRSEDTAVYYCAGSYYSYD |
| | | VLDYWGQGTTVTVSSASTKGPSVFPLAPSSKSTSGGTAALGC |
| | | LVKDYFPEPVTVSWNSGALTSGVHTFPAVLQSSGLYSLSSVV |
| | | TVPSSSLGTQTYICNVNHKPSNTKVDKRVEPKSCDKTHTCPP |
| | | CPAPELLGGPSVFLFPPKPKDTLMISRTPEVTCVVVDVSHEDP |
| | | EVKFNWYVDGVEVHNAKTKPREEQYNSTYRVVSVLTVLHQ |
| | | DWLNGKEYKCKVSNKALPAPIEKTISKAKGQPREPQVYTLPP |
| | | SREEMTKNQVSLTCLVKGFYPSDIAVEWESNGQPENNYKTTP |
| | | PVLDSDGSFFLYSKLTVDKSRWQQGNVFSCSVMHEALHNHY |
| | | TQKSLSLSPGK |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 11 | VL | DIQMTQSPSSLSASVGDRVTITCKASQNVGINVVWHQQKPGK |
| | | VPKALIYSSSHRYSGVPSRFSGSGSGTDFTLTISSLQPEDVATY |
| | | FCQQFKSYPLTFGQGTKLEIK |
| SEQ ID NO: 14 | DNA VL | GACATCCAGATGACCCAGTCTCCATCCTCTCTGTCCGCCTC |
| | | TGTGGGCGACAGAGTGACCATCACATGCAAGGCCTCTCAG |
| | | AACGTGGGCATCAACGTCGTGTGGCACCAGCAGAAACCTG |
| | | GCAAGGTGCCCAAGGCTCTGATCTACTCCTCCAGCCACAG |
| | | ATACTCCGGCGTGCCCTCTAGATTCTCCGGCTCTGGCTCTG |
| | | GCACCGACTTTACCCTGACAATCTCCAGCCTGCAGCCTGA |
| | | GGACGTGGCCACCTACTTTTGCCAGCAGTTCAAGAGCTAC |
| | | CCTCTGACCTTTGGCCAGGGCACCAAGCTGGAAATCAAG |
| SEQ ID NO: | Light chain | METDTLLLWVLLLWVPGSTGDIQMTQSPSSLSASVGDRVTIT |
| 3279 | | CKASQNVGINVVWHQQKPGKVPKALIYSSSHRYSGVPSRFSG |
| | | SGSGTDFTLTISSLQPEDVATYFCQQFKSYPLTFGQGTKLEIKR |
| | | TVAAPSVFIFPPSDEQLKSGTASVVCLLNNFYPREAKVQWKV |
| | | DNALQSGNSQESVTEQDSKDSTYSLSSTLTLSKADYEKHKVY |
| | | ACEVTHQGLSSPVTKSFNRGEC |

A-H.3 antibody

| | | |
|---|---|---|
| SEQ ID NO: 80 | VH + VL | QVQLVQSGAEVKKPGSSVKVSCKASGTDFKLTYIHWVRQAP |
| | | GQGLEWMGRVSPGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVEDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: 81 | VL | DIQMTQSPSFLSASVGDRVTITCKASQNVEDRVAWYQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 82 | VH | QVQLVQSGAEVKKPGSSVKVSCKASGTDFKLTYIHWVRQAP |
| | | GQGLEWMGRVSPGSGNTKYNEKFKGRVTITADTSTSTAYME |
| | | LSSLRSEDTAVYYCAGSYYSYDVLDYWGQGTTVTVSS |

A-H.4

| | | |
|---|---|---|
| SEQ ID NO: 83 | VH + VL | QVQLVQSGAEVKKPGSSVKVSCKASGTDFDKIYIHWVRQAP |
| | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVEDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: 84 | VL | DIQMTQSPSFLSASVGDRVTITCKASQNVEDRVAWYQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 85 | VH | QVQLVQSGAEVKKPGSSVKVSCKASGTDFDKIYIHWVRQAP |
| | | GQGLEWMGRISAGSGNVKYNEKFKGRVTITADTSTSTAYME |
| | | LSSLRSEDTAVYYCAGSYYSYDVLDYWGQGTTVTVSS |

A-H.5

| | | |
|---|---|---|
| SEQ ID NO: 86 | VH + VL | QVQLVQSGAEVKKPGSSVKVSCKASGHDFRDFYIHWVRQAP |
| | | GQGLEWMGRVYPGSGSYRYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: 87 | VL | DIQMTQSPSFLSASVGDRVTITCKASQNVDDRVAWYQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 88 | VH | QVQLVQSGAEVKKPGSSVKVSCKASGHDFRDFYIHWVRQAP |
| | | GQGLEWMGRVYPGSGSYRYNEKFKGRVTITADTSTSTAYME |
| | | LSSLRSEDTAVYYCAGSYYSYDVLDYWGQGTTVTVSS |

A-H.6

| | | |
|---|---|---|
| SEQ ID NO: 89 | VH + VL | QVQLVQSGAEVKKPGSSVKVSCKASGHDFKLTYIHWVRQAP |
| | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDNRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: 90 | VL | DIQMTQSPSFLSASVGDRVTITCKASQNVDNRVAWYQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 91 | VH | QVQLVQSGAEVKKPGSSVKVSCKASGHDFKLTYIHWVRQAP |
| | | GQGLEWMGRISAGSGNVKYNEKFKGRVTITADTSTSTAYME |
| | | LSSLRSEDTAVYYCAGSYYSYDVLDYWGQGTTVTVSS |

A-H.7

| | | |
|---|---|---|
| SEQ ID NO: 92 | VH + VL | QVQLVQSGAEVKKPGSSVKVSCKASGTDFKLTYIHWVRQAP |
| | | GQGLEWMGRIFPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVENKVAWHQQKPGKAPKALIYSSSHRYKGVPSRFSGSGS |
| | | GTEFTLTISSLQPEDFATYFCQQFKSYPLTFGQGTKLEIK |
| SEQ ID NO: 93 | VL | DIQMTQSPSFLSASVGDRVTITCKASQNVENKVAWHQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 94 | VH | QVQLVQSGAEVKKPGSSVKVSCKASGTDFKLTYIHWVRQAP |
| | | GQGLEWMGRIFPGSGNVKYNEKFKGRVTITADTSTSTAYME |
| | | LSSLRSEDTAVYYCAGSYYSYDVLDYWGQGTTVTVSS |

A-H.8

| | | |
|---|---|---|
| SEQ ID NO: 95 | VH + VL | QVQLVQSGAEVKKPGSSVKVSCKASGTDFDKIYIHWVRQAP |
| | | GQGLEWMGRIFAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: 96 | VL | DIQMTQSPSFLSASVGDRVTITCKASQNVDDRVAWYQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 97 | VH | QVQLVQSGAEVKKPGSSVKVSCKASGTDFDKIYIHWVRQAP |
| | | GQGLEWMGRIFAGSGNTKYNEKFKGRVTITADTSTSTAYME |
| | | LSSLRSEDTAVYYCAGSYYSYDVLDYWGQGTTVTVSS |

A-H.9

| | | |
|---|---|---|
| SEQ ID NO: 98 | VH + VL | QVQLVQSGAEVKKPGSSVKVSCKASGHDFDKFYIHWVRQAP |
| | | GQGLEWMGRVSAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGNRVAWYQQKPGKAPKALIYSSSHRYSGVPSRFSGSGS |
| | | GTEFTLTISSLQPEDFATYFCQQFKSYPLTFGQGTKLEIK |
| SEQ ID NO: 99 | VL | DIQMTQSPSFLSASVGDRVTITCKASQNVGNRVAWYQQKPG |
| | | KAPKALIYSSSHRYSGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFDKFYIHWVRQAP |
| 100 | | GQGLEWMGRVSAGSGNVKYNEKFKGRVTITADTSTSTAYME |
| | | LSSLRSEDTAVYYCAGSYYSYDVLDYWGQGTTVTVSS |

A-H.10

| | | |
|---|---|---|
| SEQ ID NO: | VH + VL | QVQLVQSGAEVKKPGSSVKVSCKASGHDFDKFYIHWVRQAP |
| 101 | | GQGLEWMGRIFAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVGDRVAWYQQKPG |
| 102 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFDKFYIHWVRQAP |
| 103 | | GQGLEWMGRIFAGSGNVKYNEKFKGRVTITADTSTSTAYME |
| | | LSSLRSEDTAVYYCAGSYYSYDVLDYWGQGTTVTVSS |

A-H.11

| | | |
|---|---|---|
| SEQ ID NO: | VH + VL | QVQLVQSGAEVKKPGSSVKVSCKASGTDFKLTYIHWVRQAP |
| 104 | | GQGLEWMGRVSPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVGDRVAWYQQKPG |
| 105 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 106 | | GQGLEWMGRVSPGSGNVKYNEKFKGRVTITADTSTSTAYME |
| | | LSSLRSEDTAVYYCAGSYYSYDVLDYWGQGTTVTVSS |

A-H.12

| | | |
|---|---|---|
| SEQ ID NO: | VH + VL | QVQLVQSGAEVKKPGSSVKVSCKASGTDFDKIYIHWVRQAP |
| 107 | | GQGLEWMGRVSAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGNRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVGNRVAWYQQKPG |
| 108 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFDKIYIHWVRQAP |
| 109 | | GQGLEWMGRVSAGSGNTKYNEKFKGRVTITADTSTSTAYME |
| | | LSSLRSEDTAVYYCAGSYYSYDVLDYWGQGTTVTVSS |

A-H.13, also referred to as A-H.69

| | | |
|---|---|---|
| SEQ ID NO: | VH + VL | QVQLVQSGAEVKKPGSSVKVSCKASGTDFKLTYIHWVRQAP |
| 110 | | GQGLEWMGRIFPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDNRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDNRVAWYQQKPG |
| 111 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 112 | | GQGLEWMGRIFPGSGNVKYNEKFKGRVTITADTSTSTAYME |
| | | LSSLRSEDTAVYYCAGSYYSYDVLDYWGQGTTVTVSS |

A-H.14

| | | |
|---|---|---|
| SEQ ID NO: | VH + VL | QVQLVQSGAEVKKPGSSVKVSCKASGTDFDKIYIHWVRQAP |
| 113 | | GQGLEWMGRISAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 114 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFDKIYIHWVRQAP |
| 115 | | GQGLEWMGRISAGSGNTKYNEKFKGRVTITADTSTSTAYME |
| | | LSSLRSEDTAVYYCAGSYYSYDVLDYWGQGTTVTVSS |

A-H.15

| | | |
|---|---|---|
| SEQ ID NO: | VH + VL | QVQLVQSGAEVKKPGSSVKVSCKASGTDFRLTYIHWVRQAP |
| 116 | | GQGLEWMGRVSPGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDNKVAWHQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDNKVAWHQQKPG |
| 117 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFRLTYIHWVRQAP |
| 118 | | GQGLEWMGRVSPGSGNTKYNEKFKGRVTITADTSTSTAYME |
| | | LSSLRSEDTAVYYCAGSYYSYDVLDYWGQGTTVTVSS |

A-H.16

| | | |
|---|---|---|
| SEQ ID NO: | VH + VL | QVQLVQSGAEVKKPGSSVKVSCKASGGTFRLTYIHWVRQAP |
| 119 | | GQGLEWMGRVYPGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 120 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGGTFRLTYIHWVRQAP |
| 121 | | GQGLEWMGRVYPGSGNTKYNEKFKGRVTITADTSTSTAYME |
| | | LSSLRSEDTAVYYCAGSYYSYDVLDYWGQGTTVTVSS |

A-H.17

| | | |
|---|---|---|
| SEQ ID NO: | VH + VL | QVQLVQSGAEVKKPGSSVKVSCKASGTDFRLTYIHWVRQAP |
| 122 | | GQGLEWMGRIFPGSGNTKYNEKFKGRVTITADTSTSTAYMEL |
| | | SSLRSEDTAVYYCAGSYYSYDVLDYWGQGTTVTVSSGGGGS |
| | | GGGGSGGGGSGGGGSDIQMTQSPSFLSASVGDRVTITCKASQ |
| | | NVDDRVAWYQQKPGKAPKALIYSSSHRYKGVPSRFSGSGSG |
| | | TEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 123 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFRLTYIHWVRQAP |
| 124 | | GQGLEWMGRIFPGSGNTKYNEKFKGRVTITADTSTSTAYMEL |
| | | SSLRSEDTAVYYCAGSYYSYDVLDYWGQGTTVTVSS |

A-H.18

| | | |
|---|---|---|
| SEQ ID NO: | VH + VL | QVQLVQSGAEVKKPGSSVKVSCKASGTDFKLTYIHWVRQAP |
| 125 | | GQGLEWMGRIFPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVEDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVEDRVAWYQQKPG |
| 126 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 127 | | GQGLEWMGRIFPGSGNVKYNEKFKGRVTITADTSTSTAYME |
| | | LSSLRSEDTAVYYCAGSYYSYDVLDYWGQGTTVTVSS |

A-H.19

| | | |
|---|---|---|
| SEQ ID NO: | VH + VL | QVQLVQSGAEVKKPGSSVKVSCKASGGTFRLTYIHWVRQAP |
| 128 | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVGDRVAWYQQKPG |
| 129 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGGTFRLTYIHWVRQAP |
| 130 | | GQGLEWMGRISAGSGNVKYNEKFKGRVTITADTSTSTAYME |
| | | LSSLRSEDTAVYYCAGSYYSYDVLDYWGQGTTVTVSS |

A-H.20

| | | |
|---|---|---|
| SEQ ID NO: | VH + VL | QVQLVQSGAEVKKPGSSVKVSCKASGGTFDKTYIHWVRQAP |
| 131 | | GQGLEWMGRISAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 132 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGGTFDKTYIHWVRQAP |
| 133 | | GQGLEWMGRISAGSGNTKYNEKFKGRVTITADTSTSTAYME |
| | | LSSLRSEDTAVYYCAGSYYSYDVLDYWGQGTTVTVSS |

A-H.21

| | | |
|---|---|---|
| SEQ ID NO: | VH + VL | QVQLVQSGAEVKKPGSSVKVSCKASGHDFDKFYIHWVRQAP |
| 134 | | GQGLEWMGRISAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 135 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFDKFYIHWVRQAP |
| 136 | | GQGLEWMGRISAGSGNTKYNEKFKGRVTITADTSTSTAYME |
| | | LSSLRSEDTAVYYCAGSYYSYDVLDYWGQGTTVTVSS |

A-H.22

| | | |
|---|---|---|
| SEQ ID NO: | VH + VL | QVQLVQSGAEVKKPGSSVKVSCKASGTDFKLTYIHWVRQAP |
| 137 | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDNKVAWHQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDNKVAWHQQKPG |
| 138 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 139 | | GQGLEWMGRISAGSGNVKYNEKFKGRVTITADTSTSTAYME |
| | | LSSLRSEDTAVYYCAGSYYSYDVLDYWGQGTTVTVSS |

A-H.23

| | | |
|---|---|---|
| SEQ ID NO: | VH + VL | QVQLVQSGAEVKKPGSSVKVSCKASGHDFRLTYIHWVRQAP |
| 140 | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVADRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVADRVAWYQQKPG |
| 141 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFRLTYIHWVRQAP |
| 142 | | GQGLEWMGRISAGSGNVKYNEKFKGRVTITADTSTSTAYME |
| | | LSSLRSEDTAVYYCAGSYYSYDVLDYWGQGTTVTVSS |

A-H.24

| | | |
|---|---|---|
| SEQ ID NO: | VH + VL | QVQLVQSGAEVKKPGSSVKVSCKASGHDFHLWYIHWVRQA |
| 143 | | PGQGLEWMGRVSAGSGNVKYNEKFKGRVTITADTSTSTAYM |
| | | ELSSLRSEDTAVYYCAGSYYSYDVLDYWGQGTTVTVSSGGG |
| | | GSGGGGGGGGSGGGGSDIQMTQSPSFLSASVGDRVTITCKA |
| | | SQNVDNKVAWHQQKPGKAPKALIYSSSHRYKGVPSRFSGSG |
| | | SGTEFTLTISSLQPEDFATYFCQQFKSYPLTFGQGTKLEIK |
| SEQ ID NO: | VL | DIQMTQSPSFLSASVGDRVTITCKASQNVDNKVAWHQQKPG |
| 144 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFHLWYIHWVRQA |
| 145 | | PGQGLEWMGRVSAGSGNVKYNEKFKGRVTITADTSTSTAYM |
| | | ELSSLRSEDTAVYYCAGSYYSYDVLDYWGQGTTVTVSS |

A-H.25

| | | |
|---|---|---|
| SEQ ID NO: | VH + VL | QVQLVQSGAEVKKPGSSVKVSCKASGHDFHLWYIHWVRQA |
| 146 | | PGQGLEWMGRVFAGSGNTKYNEKFKGRVTITADTSTSTAYM |
| | | ELSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVEDKVAWYQQKPGKAPKALIYSSSHRYKGVPSRFSGSG |
| | | SGTEFTLTISSLQPEDFATYFCQQFKSYPLTFGQGTKLEIK |
| SEQ ID NO: | VL | DIQMTQSPSFLSASVGDRVTITCKASQNVEDKVAWYQQKPG |
| 147 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFHLWYIHWVRQA |
| 148 | | PGQGLEWMGRVFAGSGNTKYNEKFKGRVTITADTSTSTAYM |
| | | ELSSLRSEDTAVYYCAGSYYSYDVLDYWGQGTTVTVSS |

A-H.26

| | | |
|---|---|---|
| SEQ ID NO: | VH + VL | QVQLVQSGAEVKKPGSSVKVSCKASGTDFKLTYIHWVRQAP |
| 149 | | GQGLEWMGRIFPGSGNTKYNEKFKGRVTITADTSTSTAYMEL |
| | | SSLRSEDTAVYYCAGSYYSYDVLDYWGQGTTVTVSSGGGGS |
| | | GGGGSGGGGSGGGGSDIQMTQSPSFLSASVGDRVTITCKASQ |
| | | NVDDRVAWYQQKPGKAPKALIYSSSHRYKGVPSRFSGSGSG |
| | | TEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 150 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 151 | | GQGLEWMGRIFPGSGNTKYNEKFKGRVTITADTSTSTAYMEL |
| | | SSLRSEDTAVYYCAGSYYSYDVLDYWGQGTTVTVSS |

A-H.27

| | | |
|---|---|---|
| SEQ ID NO: | VH + VL | QVQLVQSGAEVKKPGSSVKVSCKASGTDFKLTYIHWVRQAP |
| 153 | | GQGLEWMGRVSAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGNRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVGNRVAWYQQKPG |
| 154 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 155 | | GQGLEWMGRVSAGSGNVKYNEKFKGRVTITADTSTSTAYME |
| | | LSSLRSEDTAVYYCAGSYYSYDVLDYWGQGTTVTVSS |

A-H.28

| | | |
|---|---|---|
| SEQ ID NO: | VH + VL | QVQLVQSGAEVKKPGSSVKVSCKASGTDFKLTYIHWVRQAP |
| 156 | | GQGLEWMGRISPGSGNTKYNEKFKGRVTITADTSTSTAYMEL |
| | | SSLRSEDTAVYYCAGSYYSYDVLDYWGQGTTVTVSSGGGGS |
| | | GGGGSGGGGSGGGGSDIQMTQSPSFLSASVGDRVTITCKASQ |
| | | NVGDRVAWYQQKPGKAPKALIYSSSHRYKGVPSRFSGSGSG |
| | | TEFTLTISSLQPEDFATYFCQQFKSYPLTFGQGTKLEIK |
| SEQ ID NO: | VL | DIQMTQSPSFLSASVGDRVTITCKASQNVGDRVAWYQQKPG |
| 157 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 158 | | GQGLEWMGRISPGSGNTKYNEKFKGRVTITADTSTSTAYMEL |
| | | SSLRSEDTAVYYCAGSYYSYDVLDYWGQGTTVTVSS |

A-H.29

| | | |
|---|---|---|
| SEQ ID NO: | VH + VL | QVQLVQSGAEVKKPGSSVKVSCKASGHDFHLWYIHWVRQA |
| 159 | | PGQGLEWMGRISPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGDRVAWHQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVGDRVAWHQQKPG |
| 160 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFHLWYIHWVRQA |
| 161 | | PGQGLEWMGRISPGSGNVKYNEKFKGRVTITADTSTSTAYME |
| | | LSSLRSEDTAVYYCAGSYYSYDVLDYWGQGTTVTVSS |

A-H.31

| | | |
|---|---|---|
| SEQ ID NO: | VH + VL | QVQLVQSGAEVKKPGSSVKVSCKASGHDFKLTYIHWVRQAP |
| 162 | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 163 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFKLTYIHWVRQAP |
| 164 | | GQGLEWMGRISAGSGNVKYNEKFKGRVTITADTSTSTAYME |
| | | LSSLRSEDTAVYYCAGSYYSYDVLDYWGQGTTVTVSS |

A-H.31

| | | |
|---|---|---|
| SEQ ID NO: | VH + VL | QVQLVQSGAEVKKPGSSVKVSCKASGTDFHLWYIHWVRQAP |
| 165 | | GQGLEWMGRVFAGSGSYRYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 166 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFHLWYIHWVRQAP |
| 167 | | GQGLEWMGRVFAGSGSYRYNEKFKGRVTITADTSTSTAYME |
| | | LSSLRSEDTAVYYCAGSYYSYDVLDYWGQGTTVTVSS |

A-H.32

| | | |
|---|---|---|
| SEQ ID NO: | VH + VL | QVQLVQSGAEVKKPGSSVKVSCKASGTDFDKIYIHWVRQAP |
| 168 | | GQGLEWMGRISAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVADRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVADRVAWYQQKPG |
| 169 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFDKIYIHWVRQAP |
| 170 | | GQGLEWMGRISAGSGNTKYNEKFKGRVTITADTSTSTAYME |
| | | LSSLRSEDTAVYYCAGSYYSYDVLDYWGQGTTVTVSS |

A-H.33

| | | |
|---|---|---|
| SEQ ID NO: | VH + VL | QVQLVQSGAEVKKPGSSVKVSCKASGTDFKLTYIHWVRQAP |
| 171 | | GQGLEWMGRISAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVEDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVEDRVAWYQQKPG |
| 172 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 173 | | GQGLEWMGRISAGSGNTKYNEKFKGRVTITADTSTSTAYME |
| | | LSSLRSEDTAVYYCAGSYYSYDVLDYWGQGTTVTVSS |

A-H.34

| | | |
|---|---|---|
| SEQ ID NO: | VH + VL | QVQLVQSGAEVKKPGSSVKVSCKASGTDFRLTYIHWVRQAP |
| 174 | | GQGLEWMGRISPGSGNTKYNEKFKGRVTITADTSTSTAYMEL |
| | | SSLRSEDTAVYYCAGSYYSYDVLDYWGQGTTVTVSSGGGGS |
| | | GGGGSGGGGSGGGGSDIQMTQSPSFLSASVGDRVTITCKASQ |
| | | NVGNRVAWYQQKPGKAPKALIYSSSHRYKGVPSRFSGSGSG |
| | | TEFTLTISSLQPEDFATYFCQQFKSYPLTFGQGTKLEIK |
| SEQ ID NO: | VL | DIQMTQSPSFLSASVGDRVTITCKASQNVGNRVAWYQQKPG |
| 175 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFRLTYIHWVRQAP |
| 176 | | GQGLEWMGRISPGSGNTKYNEKFKGRVTITADTSTSTAYMEL |
| | | SSLRSEDTAVYYCAGSYYSYDVLDYWGQGTTVTVSS |

A-H.35

| | | |
|---|---|---|
| SEQ ID NO: | VH + VL | QVQLVQSGAEVKKPGSSVKVSCKASGHDEDKTYIHWVRQAP |
| 177 | | GQGLEWMGRVSAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVEDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVEDRVAWYQQKPG |
| 178 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDEDKTYIHWVRQAP |
| 179 | | GQGLEWMGRVSAGSGNVKYNEKFKGRVTITADTSTSTAYME |
| | | LSSLRSEDTAVYYCAGSYYSYDVLDYWGQGTTVTVSS |

A-H.36

| | | |
|---|---|---|
| SEQ ID NO: | VH + VL | QVQLVQSGAEVKKPGSSVKVSCKASGHDFKLTYIHWVRQAP |
| 180 | | GQGLEWMGRVSPGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVEDRVAWHQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVEDRVAWHQQKPG |
| 181 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFKLTYIHWVRQAP |
| 182 | | GQGLEWMGRVSPGSGNTKYNEKFKGRVTITADTSTSTAYME |
| | | LSSLRSEDTAVYYCAGSYYSYDVLDYWGQGTTVTVSS |

A-H.37

| | | |
|---|---|---|
| SEQ ID NO: | VH + VL | QVQLVQSGAEVKKPGSSVKVSCKASGHDFDKTYIHWVRQAP |
| 183 | | GQGLEWMGRIYPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVADRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVADRVAWYQQKPG |
| 184 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFDKTYIHWVRQAP |
| 185 | | GQGLEWMGRIYPGSGNVKYNEKFKGRVTITADTSTSTAYME |
| | | LSSLRSEDTAVYYCAGSYYSYDVLDYWGQGTTVTVSS |

A-H.38

| | | |
|---|---|---|
| SEQ ID NO: | VH + VL | QVQLVQSGAEVKKPGSSVKVSCKASGTDEDKTYIHWVRQAP |
| 186 | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 187 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFDKTYIHWVRQAP |
| 188 | | GQGLEWMGRISAGSGNVKYNEKFKGRVTITADTSTSTAYME |
| | | LSSLRSEDTAVYYCAGSYYSYDVLDYWGQGTTVTVSS |

A-H.39

| | | |
|---|---|---|
| SEQ ID NO: | VH + VL | QVQLVQSGAEVKKPGSSVKVSCKASGTDFDKIYIHWVRQAP |
| 189 | | GQGLEWMGRISAGSGNIKYNEKFKGRVTITADTSTSTAYMEL |
| | | SSLRSEDTAVYYCAGSYYSYDVLDYWGQGTTVTVSSGGGGS |
| | | GGGGSGGGGSGGGGSDIQMTQSPSFLSASVGDRVTITCKASQ |
| | | NVDDRVAWYQQKPGKAPKALIYSSSHRYKGVPSRFSGSGSG |
| | | TEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 190 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFDKIYIHWVRQAP |
| 191 | | GQGLEWMGRISAGSGNIKYNEKFKGRVTITADTSTSTAYMEL |
| | | SSLRSEDTAVYYCAGSYYSYDVLDYWGQGTTVTVSS |

A-H.40

| | | |
|---|---|---|
| SEQ ID NO: | VH + VL | QVQLVQSGAEVKKPGSSVKVSCKASGTDFDKIYIHWVRQAP |
| 192 | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVGDRVAWYQQKPG |
| 193 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFDKIYIHWVRQAP |
| 194 | | GQGLEWMGRISAGSGNVKYNEKFKGRVTITADTSTSTAYME |
| | | LSSLRSEDTAVYYCAGSYYSYDVLDYWGQGTTVTVSS |

A-H.41

| | | |
|---|---|---|
| SEQ ID NO: | VH + VL | QVQLVQSGAEVKKPGSSVKVSCKASGGTFKLTYIHWVRQAP |
| 195 | | GQGLEWMGRVSAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 196 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGGTFKLTYIHWVRQAP |
| 197 | | GQGLEWMGRVSAGSGNVKYNEKFKGRVTITADTSTSTAYME |
| | | LSSLRSEDTAVYYCAGSYYSYDVLDYWGQGTTVTVSS |

A-H.42

| | | |
|---|---|---|
| SEQ ID NO: | VH + VL | QVQLVQSGAEVKKPGSSVKVSCKASGTDFKLTYIHWVRQAP |
| 198 | | GQGLEWMGRISPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDNRVAWHQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDNRVAWHQQKPG |
| 199 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 200 | | GQGLEWMGRISPGSGNVKYNEKFKGRVTITADTSTSTAYME |
| | | LSSLRSEDTAVYYCAGSYYSYDVLDYWGQGTTVTVSS |

A-H.43

| | | |
|---|---|---|
| SEQ ID NO: | VH + VL | QVQLVQSGAEVKKPGSSVKVSCKASGHDFDKFYIHWVRQAP |
| 201 | | GQGLEWMGRVSAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDNRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDNRVAWYQQKPG |
| 202 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDEDKFYIHWVRQAP |
| 203 | | GQGLEWMGRVSAGSGNTKYNEKFKGRVTITADTSTSTAYME |
| | | LSSLRSEDTAVYYCAGSYYSYDVLDYWGQGTTVTVSS |

A-H.44

| | | |
|---|---|---|
| SEQ ID NO: | VH + VL | QVQLVQSGAEVKKPGSSVKVSCKASGTDFDKFYIHWVRQAP |
| 204 | | GQGLEWMGRVSAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGDRVVWYQQKPGKAPKALIYSSSHRYKGVPSRFSGSGS |
| | | GTEFTLTISSLQPEDFATYFCQQFKSYPLTFGQGTKLEIK |
| SEQ ID NO: | VH | QVQLVQSGAEVKKPGSSVKVSCKASGTDFDKFYIHWVRQAP |
| 205 | | GQGLEWMGRVSAGSGNVKYNEKFKGRVTITADTSTSTAYME |
| | | LSSLRSEDTAVYYCAGSYYSYDVLDYWGQGTTVTVSS |

A-H.45

| | | |
|---|---|---|
| SEQ ID NO: | VH + VL | QVQLVQSGAEVKKPGSSVKVSCKASGYSFTTYYIHWVRQAP |
| 206 | | GQGLEWMGWFSAGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGSG |
| | | TEFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 207 | | GQGLEWMGWFSAGSGNTKYNEKFKGRVTITADTSTSTAYME |
| | | LSSLRSEDTAVYYCAVSYYSYDVLDYWGQGTTVTVSS |

A-H.46

| | | |
|---|---|---|
| SEQ ID NO: | VH + VL | QVQLVQSGAEVKKPGSSVKVSCKASGYSFTTYYIHWVRQAP |
| 208 | | GQGLEWMGWFSAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGSG |
| | | TEFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 209 | | GQGLEWMGWFSAGSGNTKYNEKFKGRVTITADTSTSTAYME |
| | | LSSLRSEDTAVYYCAGSYYSYDVLDYWGQGTTVTVSS |

A-H.47

| | | |
|---|---|---|
| SEQ ID NO: | VH + VL | QVQLVQSGAEVKKPGSSVKVSCKASGYSFTTYYIHWVRQAP |
| 210 | | GQGLEWMGWFFPGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGSG |
| | | TEFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 211 | | GQGLEWMGWFFPGSGNTKYNEKFKGRVTITADTSTSTAYME |
| | | LSSLRSEDTAVYYCAGSYYSYDVLDYWGQGTTVTVSS |

A-H.48

| | | |
|---|---|---|
| SEQ ID NO: | VH + VL | QVQLVQSGAEVKKPGSSVKVSCKASGYSFTTYYIHWVRQAP |
| 212 | | GQGLEWMGWFSPGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGSG |
| | | TEFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 213 | | GQGLEWMGWFSPGSGNTKYNEKFKGRVTITADTSTSTAYME |
| | | LSSLRSEDTAVYYCAVSYYSYDVLDYWGQGTTVTVSS |

A-H.49

| | | |
|---|---|---|
| SEQ ID NO: | VH + VL | QVQLVQSGAEVKKPGSSVKVSCKASGYSFTTYYIHWVRQAP |
| 214 | | GQGLEWMGWFSPGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGSG |
| | | TEFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 215 | | GQGLEWMGWFSPGSGNTKYNEKFKGRVTITADTSTSTAYME |
| | | LSSLRSEDTAVYYCAGSYYSYDVLDYWGQGTTVTVSS |

A-H.50

| | | |
|---|---|---|
| SEQ ID NO: | VH + VL | QVQLVQSGAEVKKPGSSVKVSCKASGYSFTTYYIHWVRQAP |
| 216 | | GQGLEWMGRIFPGSGNIKYNEKFKGRVTITADTSTSTAYMEL |
| | | SSLRSEDTAVYYCAGSYYSYDVLDYWGQGTTVTVSSGGGGS |
| | | GGGGSGGGGSGGGGSDIQMTQSPSFLSASVGDRVTITCKASQ |
| | | NVGINVVWHQQKPGKAPKALIYSSSHRYSGVPSRFSGSGSGT |
| | | EFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 217 | | GQGLEWMGRIFPGSGNIKYNEKFKGRVTITADTSTSTAYMEL |
| | | SSLRSEDTAVYYCAGSYYSYDVLDYWGQGTTVTVSS |

A-H.51

| | | |
|---|---|---|
| SEQ ID NO: | VH + VL | QVQLVQSGAEVKKPGSSVKVSCKASGYSFTTYYIHWVRQAP |
| 218 | | GQGLEWMGWFFPGSGNIKYNEKFKGRVTITADTSTSTAYME |
| | | LSSLRSEDTAVYYCAGSIYSAGVLDYWGQGTTVTVSSGGGGS |
| | | GGGGSGGGGSGGGGSDIQMTQSPSFLSASVGDRVTITCKASQ |
| | | NVGINVVWHQQKPGKAPKALIYSSSHRYSGVPSRFSGSGSGT |
| | | EFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 219 | | GQGLEWMGWFFPGSGNIKYNEKFKGRVTITADTSTSTAYME |
| | | LSSLRSEDTAVYYCAGSIYSAGVLDYWGQGTTVTVSS |

A-H.52

| | | |
|---|---|---|
| SEQ ID NO: | VH + VL | QVQLVQSGAEVKKPGSSVKVSCKASGYSFTLGYIHWVRQAP |
| 220 | | GQGLEWMGWFFPGSGNIKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGSG |
| | | TEFTLTISSLQPEDFATYFCQQFKSYPLTFGQGTKLEIK |
| SEQ ID NO: | VH | QVQLVQSGAEVKKPGSSVKVSCKASGYSFTLGYIHWVRQAP |
| 221 | | GQGLEWMGWFFPGSGNIKYNEKFKGRVTITADTSTSTAYME |
| | | LSSLRSEDTAVYYCAGSYYSYDVLDYWGQGTTVTVSS |

A-H.53

| | | |
|---|---|---|
| SEQ ID NO: | VH + VL | QVQLVQSGAEVKKPGSSVKVSCKASGYSFRLTYIHWVRQAP |
| 222 | | GQGLEWMGWFFPGSGNIKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGSG |
| | | TEFTLTISSLQPEDFATYFCQQFKSYPLTFGQGTKLEIK |
| SEQ ID NO: | VH | QVQLVQSGAEVKKPGSSVKVSCKASGYSFRLTYIHWVRQAP |
| 223 | | GQGLEWMGWFFPGSGNIKYNEKFKGRVTITADTSTSTAYME |
| | | LSSLRSEDTAVYYCAGSYYSYDVLDYWGQGTTVTVSS |

A-H.54

| | | |
|---|---|---|
| SEQ ID NO: | VH + VL | QVQLVQSGAEVKKPGSSVKVSCKASGYSFHNWYIHWVRQA |
| 224 | | PGQGLEWMGWFFPGSGNIKYNEKFKGRVTITADTSTSTAYM |
| | | ELSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVGINVVWHQQKPGKAPKALIYSSSHRYSGVPSRFSGSGS |
| | | GTEFTLTISSLQPEDFATYFCQQFKSYPLTFGQGTKLEIK |
| SEQ ID NO: | VH | QVQLVQSGAEVKKPGSSVKVSCKASGYSFHNWYIHWVRQA |
| 225 | | PGQGLEWMGWFFPGSGNIKYNEKFKGRVTITADTSTSTAYM |
| | | ELSSLRSEDTAVYYCAGSYYSYDVLDYWGQGTTVTVSS |

A-H.55 antibody

| | | |
|---|---|---|
| SEQ ID NO: 3 | HC CDR1 (Combined) | GYSFTTYYIH |
| SEQ ID NO: 4 | HC CDR2 (Combined) | WFFPGSGNIKYNEKFKG |
| SEQ ID NO: 5 | HC CDR3 (Combined) | SYYSYDVLDY |
| SEQ ID NO: 45 | HC CDR1 (Kabat) | TYYIH |
| SEQ ID NO: 46 | HC CDR2 (Kabat) | WFFPGSGNIKYNEKFKG |
| SEQ ID NO: 47 | HC CDR3 (Kabat) | SYYSYDVLDY |
| SEQ ID NO: 48 | HC CDR1 (Chothia) | GYSFTTY |
| SEQ ID NO: 49 | HC CDR2 (Chothia) | FPGSGN |
| SEQ ID NO: 50 | HC CDR3 (Chothia) | SYYSYDVLDY |
| SEQ ID NO: | VH | QVQLVQSGAEVKKPGSSVKVSCKASGYSFTTYYIHWVRQAP |
| 1100 | | GQGLEWMGWFFPGSGNIKYNEKFKGRVTITADTSTSTAYME |
| | | LSSLRSEDTAVYYCAGSYYSYDVLDYWGQGTTVTVSS |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 51 | LC CDR1 (Kabat) | KASQNVGINVV |
| SEQ ID NO: 52 | LC CDR2 (Kabat) | SSSHRYS |
| SEQ ID NO: 53 | LC CDR3 (Kabat) | QQFKSYPLT |
| SEQ ID NO: 54 | LC CDR1 (Chothia) | KASQNVGINVV |
| SEQ ID NO: 55 | LC CDR2 (chothia) | SSSHRYS |
| SEQ ID NO: 56 | LC CDR3 (chothia) | QQFKSYPLT |
| SEQ ID NO: | VL | QSVLTQPPSVSEAPRQRVTISCKASQNVGINVVWHQQLPGKA |
| 1101 | | PKALIYSSSHRYSGVSDRFSGSGSGTSFSLAISGLQSEDEADYF |
| | | CQQFKSYPLTFGTGTKVTVL |

A-H.56

| | | |
|---|---|---|
| SEQ ID NO: | VH + VL (ScFv) | QVQLVQSGAEVKKPGSSVKVSCKASGHDFDKFYIHWVRQAP |
| 1309 | | GQGLEWMGRVSAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGNRVAWYQQKPGKAPKALIYSSSHRYKGVPSRFSGSGS |
| | | GTEFTLTISSLQPEDFATYFCQQFKSYPLTFGQGTKLEIK |

A-H.57

| | | |
|---|---|---|
| SEQ ID NO: | VH + VL (ScFv) | QVQLVQSGAEVKKPGSSVKVSCKASGHDFRLTYIHWVRQAP |
| 1326 | | GQGLEWMGRVSAGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVGDRVVWHQQKPGKAPKALIYSSSHRYKGVPSRFSGSGS |
| | | GTEFTLTISSLQPEDFATYFCQQFKSYPLTFGQGTKLEIK |

A-H.58

| | | |
|---|---|---|
| SEQ ID NO: | VH + VL (ScFv) | QVQLVQSGAEVKKPGSSVKVSCKASGHDFRLTYIHWVRQAP |
| 1327 | | GQGLEWMGRVSAGSGNVKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVGNRVVWHQQKPGKAPKALIYSSSHRYKGVPSRFSGSGS |
| | | GTEFTLTISSLQPEDFATYFCQQFKSYPLTFGQGTKLEIK |

A-H.59

| | | |
|---|---|---|
| SEQ ID NO: | VH + VL (ScFv) | QVQLVQSGAEVKKPGSSVKVSCKASGHDFRLTYIHWVRQAP |
| 1328 | | GQGLEWMGRIYAGSGNVKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVADRVVWHQQKPGKAPKALIYSSSHRYKGVPSRFSGSGS |
| | | GTEFTLTISSLQPEDFATYFCQQFKSYPLTFGQGTKLEIK |

A-H.60

| | | |
|---|---|---|
| SEQ ID NO: | VH + VL (ScFv) | QVQLVQSGAEVKKPGSSVKVSCKASGHDFKLTYIHWVRQAP |
| 1329 | | GQGLEWMGRVSAGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVGDRVAWHQQKPGKAPKALIYSSSHRYKGVPSRFSGSGS |
| | | GTEFTLTISSLQPEDFATYFCQQFKSYPLTFGQGTKLEIK |

A-H.61

| | | |
|---|---|---|
| SEQ ID NO: | VH + VL (ScFv) | QVQLVQSGAEVKKPGSSVKVSCKASGHDFKLTYIHWVRQAP |
| 1330 | | GQGLEWMGRVSAGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVDNRVAWHQQKPGKAPKALIYSSSHRYKGVPSRFSGSGS |
| | | GTEFTLTISSLQPEDFATYFCQQFKSYPLTFGQGTKLEIK |

A-H.62

| | | |
|---|---|---|
| SEQ ID NO: | VH + VL (ScFv) | QVQLVQSGAEVKKPGSSVKVSCKASGHDFRLTYIHWVRQAP |
| 1331 | | GQGLEWMGRVSAGSGNVKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVADRVVWHQQKPGKAPKALIYSSSHRYKGVPSRFSGSGS |
| | | GTEFTLTISSLQPEDFATYFCQQFKSYPLTFGQGTKLEIK |

A-H.63

| | | |
|---|---|---|
| SEQ ID NO: | VH + VL (ScFv) | QVQLVQSGAEVKKPGSSVKVSCKASGHDFRLTYIHWVRQAP |
| 1332 | | GQGLEWMGRVYAGSGNTKYNEKFKGRVTITADTSTSTAYM |
| | | ELSSLRSEDTAVYYCAVSYYSYDVLDYWGQGTTVTVSSGGG |
| | | GSGGGGSGGGGSGGGGSDIQMTQSPSFLSASVGDRVTITCKA |
| | | SQNVEDRVVWHQQKPGKAPKALIYSSSHRYKGVPSRFSGSGS |
| | | GTEFTLTISSLQPEDFATYFCQQFKSYPLTFGQGTKLEIK |

A-H.64

| | | |
|---|---|---|
| SEQ ID NO: | VH + VL (ScFv) | QVQLVQSGAEVKKPGSSVKVSCKASGHDFKLTYIHWVRQAP |
| 1333 | | GQGLEWMGRVSAGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVADRVVWHQQKPGKAPKALIYSSSHRYKGVPSRFSGSGS |
| | | GTEFTLTISSLQPEDFATYFCQQFKSYPLTFGQGTKLEIK |

A-H.65

| | | |
|---|---|---|
| SEQ ID NO: | VH + VL (ScFv) | QVQLVQSGAEVKKPGSSVKVSCKASGHDFKLTYIHWVRQAP |
| 1334 | | GQGLEWMGRISAGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVGDRVVWHQQKPGKAPKALIYSSSHRYKGVPSRFSGSGS |
| | | GTEFTLTISSLQPEDFATYFCQQFKSYPLTFGQGTKLEIK |

A-H.66

| | | |
|---|---|---|
| SEQ ID NO: | VH + VL (ScFv) | QVQLVQSGAEVKKPGSSVKVSCKASGHDFKLTYIHWVRQAP |
| 1335 | | GQGLEWMGRIYAGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVGDRVVWHQQKPGKAPKALIYSSSHRYKGVPSRFSGSGS |
| | | GTEFTLTISSLQPEDFATYFCQQFKSYPLTFGQGTKLEIK |

A-H.67

| | | |
|---|---|---|
| SEQ ID NO: | VH + VL (ScFv) | QVQLVQSGAEVKKPGSSVKVSCKASGTDFKLTYIHWVRQAP |
| 1336 | | GQGLEWMGRIFPGSGNVKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVDNRVAWYQQKPGKAPKALIYSSSHRYKGVPSRFSGSGS |
| | | GTEFTLTISSLQPEDFATYFCQQFKSYPLTFGQGTKLEIK |

A-H.68

| | | |
|---|---|---|
| SEQ ID NO: | VH + VL (ScFv) | QVQLVQSGAEVKKPGSSVKVSCKASGHDFRLTYIHWVRQAP |
| 1337 | | GQGLEWMGRISAGSGNVKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVADRVAWYQQKPGKAPKALIYSSSHRYKGVPSRFSGSGS |
| | | GTEFTLTISSLQPEDFATYFCQQFKSYPLTFGQGTKLEIK |

A-H.69 (also referred to as A-H.13)

| | | |
|---|---|---|
| SEQ ID NO: | VH + VL (ScFv) | QVQLVQSGAEVKKPGSSVKVSCKASGTDFKLTYIHWVRQAP |
| 110 | | GQGLEWMGRIFPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDNRVAWYQQKPGKAPKALIYSSSHRYKGVPSRFSGSGS |
| | | GTEFTLTISSLQPEDFATYFCQQFKSYPLTFGQGTKLEIK |

A-H humanized-matured VH

| | | |
|---|---|---|
| SEQ ID NO: | VH-humanized matured | QVQLVQSGAEVKKPGSSVKVSCKASGTDFKLTYIHWVRQAP |
| 1310 | 1 | GQGLEWMGRIFPGSGNVKYNEKFKGRVTITADTSTSTAYME |
| | | LSSLRSEDTAVYYCAGSYYSYDVLDYWGQGTTVTVSS |
| SEQ ID NO: | VH-humanized matured | QVQLVQSGAEVKKPGSSVKVSCKASGTDFKLTYIHWVRQAP |
| 1311 | 2 | GQGLEWMGRIFPGSGNVKYNEKFKGRVTITADTSTSTAYME |
| | | LSSLRSEDTAVYYCAVSYYSYDVLDYWGQGTTVTVSS |
| SEQ ID NO: | VH-humanized matured | QVQLVQSGAEVKKPGSSVKVSCKASGHDFRLTYIHWVRQAP |
| 1312 | 3 | GQGLEWMGRISAGSGNVKYNEKFKGRVTITADTSTSTAYME |
| | | LSSLRSEDTAVYYCAVSYYSYDVLDYWGQGTTVTVSS |

A-H humanized-matured VL

| | | |
|---|---|---|
| SEQ ID NO: | VL-humanized matured | DIQMTQSPSFLSASVGDRVTITCKASQNVDNRVAWYQQKPG |
| 1313 | 1 | KAPKALIYSSSHRYKGVPSRFSGSGSGTEFTLTISSLQPEDFAT |
| | | YFCQQFKSYPLTFGQGTKLEIK |
| SEQ ID NO: | VL-humanized matured | DIQMTQSPSFLSASVGDRVTITCKASQNVADRVAWYQQKPG |
| 1314 | 2 | KAPKALIYSSSHRYKGVPSRFSGSGSGTEFTLTISSLQPEDFAT |
| | | YFCQQFKSYPLTFGQGTKLEIK |

A-H.70

| | | |
|---|---|---|
| SEQ ID NO: | HC CDR1 | GHDFRLTYIH |
| 3650 | | |
| SEQ ID NO: | HC CDR2 | RVSAGSGNVKYNEKFKG |
| 3651 | | |
| SEQ ID NO: 5 | HC CDR3 | SYYSYDVLDY |
| SEQ ID NO: | LC CDR1 | KASQNVGNRVV |
| 3652 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFRLTYIHWVRQAP |
| 1346 | (CDRs underlined) | GQGLEWMGRVSAGSGNVKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVGNRVVWHQQKPG |
| 1347 | (CDRs underlined) | KAPKALIYSSSHRYKGVPSRFSGSGSGTEFTLTISSLQPEDFAT |
| | | YFCQQFKSYPLTFGQGTKLEIK |

A-H.71

| | | |
|---|---|---|
| SEQ ID NO: | HC CDR1 | GHDFRLTYIH |
| 3650 | | |
| SEQ ID NO: | HC CDR2 | RIYAGSGNVKYNEKFKG |
| 3654 | | |
| SEQ ID NO: 5 | HC CDR3 | SYYSYDVLDY |
| SEQ ID NO: | LC CDR1 | KASQNVADRVV |
| 3655 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFRLTYIHWVRQAP |
| 1348 | (CDRs underlined) | GQGLEWMGRIYAGSGNVKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVADRVVWHQQKPG |
| 1349 | (CDRs underlined) | KAPKALIYSSSHRYKGVPSRFSGSGSGTEFTLTISSLQPEDFAT |
| | | YFCQQFKSYPLTFGQGTKLEIK |

A-H.72

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVGDRVA |
| 3658 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1350 | (CDRs underlined) | GQGLEWMGRVSAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVGDRVAWHQQKPG |
| 1351 | (CDRs underlined) | KAPKALIYSSSHRYKGVPSRFSGSGSGTEFTLTISSLQPEDFAT |
| | | YFCQQFKSYPLTFGQGTKLEIK |

A-H.73

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1350 | (CDRs underlined) | GQGLEWMGRVSAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVDNRVAWHQQKPG |
| 1353 | (CDRs underlined) | KAPKALIYSSSHRYKGVPSRFSGSGSGTEFTLTISSLQPEDFAT |
| | | YFCQQFKSYPLTFGQGTKLEIK |

A-H.74

| | | |
|---|---|---|
| SEQ ID NO: | HC CDR1 | GHDFRLTYIH |
| 3650 | | |
| SEQ ID NO: | HC CDR2 | RVSAGSGNVKYNEKFKG |
| 3651 | | |
| SEQ ID NO: 5 | HC CDR3 | SYYSYDVLDY |
| SEQ ID NO: | LC CDR1 | KASQNVADRVV |
| 3655 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFRLTYIHWVRQAP |
| 1346 | (CDRs underlined) | GQGLEWMGRVSAGSGNVKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVADRVVWHQQKPG |
| 1349 | (CDRs underlined) | KAPKALIYSSSHRYKGVPSRFSGSGSGTEFTLTISSLQPEDFAT |
| | | YFCQQFKSYPLTFGQGTKLEIK |

A-H.75

| | | |
|---|---|---|
| SEQ ID NO: | HC CDR1 | GHDFRLTYIH |
| 3650 | | |
| SEQ ID NO: | HC CDR2 | RVYAGSGNTKYNEKFKG |
| 3659 | | |
| SEQ ID NO: 5 | HC CDR3 | SYYSYDVLDY |
| SEQ ID NO: | LC CDR1 | KASQNVEDRVV |
| 3660 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFRLTYIHWVRQAP |
| 1356 | (CDRs underlined) | GQGLEWMGRVYAGSGNTKYNEKFKGRVTITADTSTSTAYM |
| | | ELSSLRSEDTAVYYCAVSYYSYDVLDYWGQGTTVTVSS |
| SEQ ID NO: | VL | DIQMTQSPSFLSASVGDRVTITCKASQNVEDRVVWHQQKPG |
| 1357 | (CDRs underlined) | KAPKALIYSSSHRYKGVPSRFSGSGSGTEFTLTISSLQPEDFAT |
| | | YFCQQFKSYPLTFGQGTKLEIK |

A-H.76

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVADRVV |
| 3655 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1350 | (CDRs underlined) | GQGLEWMGRVSAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVADRVVWHQQKPG |
| 1349 | (CDRs underlined) | KAPKALIYSSSHRYKGVPSRFSGSGSGTEFTLTISSLQPEDFAT |
| | | YFCQQFKSYPLTFGQGTKLEIK |

A-H.77

| | | |
|---|---|---|
| SEQ ID NO: | HC CDR1 | GHDFKLTYIH |
| 3656 | | |
| SEQ ID NO: | HC CDR2 | RISAGSGNTKYNEKFKG |
| 3661 | | |
| SEQ ID NO: 5 | HC CDR3 | SYYSYDVLDY |
| SEQ ID NO: | LC CDR1 | KASQNVGDRVV |
| 3662 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1360 | (CDRs underlined) | GQGLEWMGRISAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVGDRVVWHQQKPG |
| 1361 | (CDRs underlined) | KAPKALIYSSSHRYKGVPSRFSGSGSGTEFTLTISSLQPEDFAT |
| | | YFCQQFKSYPLTFGQGTKLEIK |

A-H.78

| | | |
|---|---|---|
| SEQ ID NO: | HC CDR1 | GHDFKLTYIH |
| 3656 | | |
| SEQ ID NO: | HC CDR2 | RIYAGSGNTKYNEKFKG |
| 3663 | | |
| SEQ ID NO: 5 | HC CDR3 | SYYSYDVLDY |
| SEQ ID NO: | LC CDR1 | KASQNVGDRVV |
| 3662 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1362 | (CDRs underlined) | GQGLEWMGRIYAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVGDRVVWHQQKPG |
| 1361 | (CDRs underlined) | KAPKALIYSSSHRYKGVPSRFSGSGSGTEFTLTISSLQPEDFAT |
| | | YFCQQFKSYPLTFGQGTKLEIK |

A-H.79

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | RASQNVDNRLG |
| 3666 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1350 | (CDRs underlined) | GQGLEWMGRVSAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCRASQNVDNRLGWHQQKPG |
| 1365 | (CDRs underlined) | KAPKALIYSSSHRYKGVPSRFSGSGSGTEFTLTISSLQPEDFAT |
| | | YFCQQFKSYPLTFGQGTKLEIK |

A-H.80

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | AASSLQK |
| 3668 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1350 | (CDRs underlined) | GQGLEWMGRVSAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVDNRVAWHQQKPG |
| 1367 | (CDRs underlined) | KAPKALIYAASSLQKGVPSRFSGSGSGTEFTLTISSLQPEDFAT |
| | | YFCQQFKSYPLTFGQGTKLEIK |

A-H.81

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | SSSHRYK |
| 3653 | | |
| SEQ ID NO: | LC CDR3 | LQHNSYPLT |
| 3664 | | |
| SEQ ID NO: | VH | QVQLVQSGAEVKKPGSSVKVSCKASGHDFKLTYIHWVRQAP |
| 1350 | (CDRs underlined) | GQGLEWMGRVSAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVDNRVAWHQQKPG |
| 1369 | (CDRs underlined) | KAPKALIYSSSHRYKGVPSRFSGSGSGTEFTLTISSLQPEDFAT |
| | | YFCLQHNSYPLTFGQGTKLEIK |

A-H.82

| | | |
|---|---|---|
| SEQ ID NO: | HC CDR1 | GHDFKLTYIH |
| 3656 | | |
| SEQ ID NO: | HC CDR2 | RVSAGSGNVNYAQKFQG |
| 3665 | | |
| SEQ ID NO: 5 | HC CDR3 | SYYSYDVLDY |
| SEQ ID NO: | LC CDR1 | RASQNVDNRLG |
| 3666 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1370 | (CDRs underlined) | GQGLEWMGRVSAGSGNVNYAQKFQGRVTITADTSTSTAYM |
| | | ELSSLRSEDTAVYYCAVSYYSYDVLDYWGQGTTVTVSS |
| SEQ ID NO: | VL | DIQMTQSPSFLSASVGDRVTITCRASQNVDNRLGWHQQKPG |
| 1365 | (CDRs underlined) | KAPKALIYSSSHRYKGVPSRFSGSGSGTEFTLTISSLQPEDFAT |
| | | YFCQQFKSYPLTFGQGTKLEIK |

A-H.83

| | | |
|---|---|---|
| SEQ ID NO: | HC CDR1 | GHDFKLTYIH |
| 3656 | | |
| SEQ ID NO: | HC CDR2 | RVSAGSGNVNYAQKFQG |
| 3665 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | AASSLQK |
| 3668 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1370 | (CDRs underlined) | GQGLEWMGRVSAGSGNVNYAQKFQGRVTITADTSTSTAYM |
| | | ELSSLRSEDTAVYYCAVSYYSYDVLDYWGQGTTVTVSS |
| SEQ ID NO: | VL | DIQMTQSPSFLSASVGDRVTITCKASQNVDNRVAWHQQKPG |
| 1367 | (CDRs underlined) | KAPKALIYAASSLQKGVPSRFSGSGSGTEFTLTISSLQPEDFAT |
| | | YFCQQFKSYPLTFGQGTKLEIK |

A-H.84

| | | |
|---|---|---|
| SEQ ID NO: | HC CDR1 | GHDFKLTYIH |
| 3656 | | |
| SEQ ID NO: | HC CDR2 | RVSAGSGNVNYAQKFQG |
| 3665 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | SSSHRYK |
| 3653 | | |
| SEQ ID NO: | LC CDR3 | LQHNSYPLT |
| 3664 | | |
| SEQ ID NO: | VH | QVQLVQSGAEVKKPGSSVKVSCKASGHDFKLTYIHWVRQAP |
| 1370 | (CDRs underlined) | GQGLEWMGRVSAGSGNVNYAQKFQGRVTITADTSTSTAYM |
| | | ELSSLRSEDTAVYYCAVSYYSYDVLDYWGQGTTVTVSS |
| SEQ ID NO: | VL | DIQMTQSPSFLSASVGDRVTITCKASQNVDNRVAWHQQKPG |
| 1369 | (CDRs underlined) | KAPKALIYSSSHRYKGVPSRFSGSGSGTEFTLTISSLQPEDFAT |
| | | YFCLQHNSYPLTFGQGTKLEIK |

A-H.85

| | | |
|---|---|---|
| SEQ ID NO: | HC CDR1 | GHDFRLTYIH |
| 3650 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVGDRVV |
| 3662 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFRLTYIHWVRQAP |
| 1344 | (CDRs underlined) | GQGLEWMGRVSAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVGDRVVWHQQKPG |
| 1361 | (CDRs underlined) | KAPKALIYSSSHRYKGVPSRFSGSGSGTEFTLTISSLQPEDFAT |
| | | YFCQQFKSYPLTFGQGTKLEIK |

TABLE 2
Amino acid and nucleotide sequences for murine and humanized antibody molecules which bind
to TCRVB 12, e.g., TCRVB 12-3 or TCRVB 12-4. The antibody molecules include murine mAb Antibody
B and humanized mAb Antibody B-H.1 to B-H.6. The amino acid the heavy and light chain CDRs,
and the amino acid and nucleotide sequences of the heavy and light chain variable regions,
and the heavy and light chains are shown.
Antibody B (murine), also referred to as 16G8

| | | |
|---|---|---|
| SEQ ID NO: 17 | HC CDR1 (Combined) | GFTFSNFGMH |
| SEQ ID NO: 18 | HC CDR2 (Combined) | YISSGSSTIYYADTLKG |
| SEQ ID NO: 19 | HC CDR3 (Combined) | RGEGAMDY |
| SEQ ID NO: 57 | HC CDR1 (Kabat) | NFGMH |
| SEQ ID NO: 58 | HC CDR2 (Kabat) | YISSGSSTIYYADTLKG |
| SEQ ID NO: 59 | HC CDR3 (Kabat) | RGEGAMDY |
| SEQ ID NO: 60 | HC CDR1 (Chothia) | GFTFSNF |
| SEQ ID NO: 61 | HC CDR2 (Chothia) | SSGSST |
| SEQ ID NO: 62 | HC CDR3 (Chothia) | RGEGAMDY |
| SEQ ID NO: 15 | VH | DVQLVESGGGLVQPGGSRKLSCAASGFTFSNFGMHWVRQ |
| | | APDKGLEWVAYISSGSSTIYYADTLKGRFTISRDNPKNTLFL |
| | | QMTSLRSEDTAMYYCARRGEGAMDYWGQGTSVTVSS |
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3 (Combined) | QQFTSSPFT |
| SEQ ID NO: 63 | LC CDR1 (Kabat) | RASSSVNYIY |
| SEQ ID NO: 64 | LC CDR2 (Kabat) | YTSNLAP |
| SEQ ID NO: 65 | LC CDR3 (Kabat) | QQFTSSPFT |
| SEQ ID NO: 66 | LC CDR1 (Chothia) | RASSSVNYIY |
| SEQ ID NO: 67 | LC CDR2 (Chothia) | YTSNLAP |
| SEQ ID NO: 68 | LC CDR3 (Chothia) | QQFTSSPFT |
| SEQ ID NO: 16 | VL | ENVLTQSPAIMSASLGEKVTMSCRASSSVNYIYWYQQKSD |
| | | ASPKLWIYYTSNLAPGVPTRFSGSGSGNSYSLTISSMEGEDA |
| | | ATYYCQQFTSSPFTFGSGTKLEIK |

Antibody B humanized (B-H)
Antibody B-H.1A HC-1

| | | |
|---|---|---|
| SEQ ID NO: 17 | HC CDR1 (Combined) | GFTFSNFGMH |
| SEQ ID NO: 18 | HC CDR2 (Combined) | YISSGSSTIYYADTLKG |
| SEQ ID NO: 19 | HC CDR3 (Combined) | RGEGAMDY |
| SEQ ID NO: | VH | EVQLVESGGGLVQPGGSLRLSCAASGFTFSNFGMHWVRQA |
| 3438 | | PGKGLEWVSYISSGSSTIYYADTLKGRFTISRDNAKNSLYLQ |
| | | MNSLRAEDTAVYYCARRGEGAMDYWGQGTTVTVSS |
| SEQ ID NO: 31 | DNA VH | GAGGTGCAGCTGGTTGAATCTGGCGGAGGATTGGTTCAG |
| | | CCTGGCGGCTCTCTGAGACTGTCTTGTGCCGCTTCTGGCT |
| | | TCACCTTCTCCAACTTCGGCATGCACTGGGTCCGACAGG |
| | | CCCCTGGAAAAGGACTGGAATGGGTGTCCTACATCTCCT |
| | | CCGGCTCCTCCACCATCTACTACGCTGACACCCTGAAGG |
| | | GCAGATTCACCATCTCTCGGGACAACGCCAAGAACTCCC |
| | | TGTACCTGCAGATGAACAGCCTGAGAGCCGAGGACACC |
| | | GCCGTGTACTACTGTGCTAGAAGAGGCGAGGGCGCCATG |
| | | GATTATTGGGGCCAGGGAACCACAGTGACCGTGTCTAGC |

Antibody B-H. 1B HC-2

| | | |
|---|---|---|
| SEQ ID NO: 17 | HC CDR1 (Combined) | GFTFSNFGMH |
| SEQ ID NO: 18 | HC CDR2 (Combined) | YISSGSSTIYYADTLKG |
| SEQ ID NO: 19 | HC CDR3 (Combined) | RGEGAMDY |
| SEQ ID NO: 25 | VH | EVQLVESGGGLVQPGGSLRLSCAASGFTFSNFGMHWVRQA |
| | | PGKGLEWVSYISSGSSTIYYADTLKGRFTISRDNSKNTLYLQ |
| | | MNSLRAEDTAVYYCARRGEGAMDYWGQGTTVTVSS |
| SEQ ID NO: 32 | DNA VH | GAGGTGCAGCTGGTTGAATCTGGCGGAGGATTGGTTCAG |
| | | CCTGGCGGCTCTCTGAGACTGTCTTGTGCCGCTTCTGGCT |
| | | TCACCTTCTCCAACTTCGGCATGCACTGGGTCCGACAGG |
| | | CCCCTGGAAAAGGACTGGAATGGGTGTCCTACATCTCCT |
| | | CCGGCTCCTCCACCATCTACTACGCTGACACCCTGAAGG |
| | | GCAGATTCACCATCAGCCGGGACAACTCCAAGAACACCC |
| | | TGTACCTGCAGATGAACTCCCTGAGAGCCGAGGACACCG |
| | | CCGTGTACTACTGTGCTAGAAGAGGCGAGGGCGCCATGG |
| | | ATTATTGGGGCCAGGGAACCACAGTGACCGTGTCTAGC |

Antibody B-H.IC HC-3

| | | |
|---|---|---|
| SEQ ID NO: 17 | HC CDR1 (Combined) | GFTFSNFGMH |
| SEQ ID NO: 18 | HC CDR2 (Combined) | YISSGSSTIYYADTLKG |
| SEQ ID NO: 19 | HC CDR3 (Combined) | RGEGAMDY |
| SEQ ID NO: 23 | VH | QVQLVESGGGVVQPGRSLRLSCAASGFTFSNFGMHWVRQ |
| | | APGKGLEWVAYISSGSSTIYYADTLKGRFTISRDNSKNTLY |
| | | LQMNSLRAEDTAVYYCARRGEGAMDYWGQGTTVTVSS |
| SEQ ID NO: 33 | DNA VH | CAGGTGCAGCTGGTGGAATCTGGTGGCGGAGTTGTGCAG |
| | | CCTGGCAGATCCCTGAGACTGTCTTGTGCCGCCTCTGGCT |
| | | TCACCTTCTCCAACTTCGGCATGCACTGGGTCCGACAGG |
| | | CCCCTGGAAAAGGATTGGAGTGGGTCGCCTACATCTCCT |
| | | CCGGCTCCTCCACCATCTACTACGCTGACACCCTGAAGG |
| | | GCAGATTCACCATCAGCCGGGACAACTCCAAGAACACCC |
| | | TGTACCTGCAGATGAACTCCCTGAGAGCCGAGGACACCG |
| | | CCGTGTACTACTGTGCTAGAAGAGGCGAGGGCGCCATGG |
| | | ATTATTGGGGCCAGGGAACCACAGTGACCGTGTCTAGC |

Antibody B-H.ID LC-1

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3 (Combined) | QQFTSSPFT |
| SEQ ID NO: 26 | VL | DNQLTQSPSFLSASVGDRVTITCRASSSVNYIYWYQQKPGK |
| | | APKLLIYYTSNLAPGVPSRFSGSGSGNEYTLTISSLQPEDFAT |
| | | YYCQQFTSSPFTFGQGTKLEIK |
| SEQ ID NO: 34 | DNA VL | GATAACCAGCTGACCCAGTCTCCTAGCTTCCTGTCTGCCT |
| | | CTGTGGGCGACAGAGTGACAATTACCTGCCGGGCCTCCT |
| | | CCTCCGTGAACTACATCTACTGGTATCAGCAGAAGCCCG |
| | | GCAAGGCCCCTAAGCTGCTGATCTACTACACCTCCAATC |
| | | TGGCCCCTGGCGTGCCCTCTAGATTTTCCGGATCTGGCTC |
| | | CGGCAACGAGTATACCCTGACAATCTCCAGCCTGCAGCC |
| | | TGAGGACTTCGCCACCTACTACTGCCAGCAGTTCACCTC |
| | | CTCTCCATTCACCTTTGGCCAGGGCACCAAGCTGGAAAT |
| | | CAAA |

Antibody B-H.1E LC-2

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3 (Combined) | QQFTSSPFT |
| SEQ ID NO: 27 | VL | DNQLTQSPSSLSASVGDRVTITCRASSSVNYIYWYQQKPGK |
| | | APKLLIYYTSNLAPGVPSRFSGSGSGNDYTLTISSLQPEDFA |
| | | TYYCQQFTSSPFTFGQGTKLEIK |
| SEQ ID NO: 35 | DNA VL | ATAACCAGCTGACCCAGTCTCCTTCCAGCCTGTCTGCTTC |
| | | TGTGGGCGACAGAGTGACAATTACCTGCCGGGCCTCCTC |
| | | CTCCGTGAACTACATCTACTGGTATCAGCAGAAGCCCGG |
| | | CAAGGCCCCTAAGCTGCTGATCTACTACACCTCCAATCT |
| | | GGCCCCTGGCGTGCCCTCTAGATTTTCCGGATCTGGCTCC |
| | | GGCAACGACTATACCCTGACAATCTCCAGCCTGCAGCCT |
| | | GAGGACTTCGCCACCTACTACTGCCAGCAGTTCACCTCC |
| | | TCTCCATTCACCTTTGGCCAGGGCACCAAGCTGGAAATC |
| | | AAA |

Antibody B-H.IF LC-3

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3 (Combined) | QQFTSSPFT |
| SEQ ID NO: 28 | VL | ENVLTQSPATLSVSPGERATLSCRASSSVNYIYWYQQKPGQ |
| | | APRLLIYYTSNLAPGIPARFSGSGSGNEYTLTISSLQSEDFAV |
| | | YYCQQFTSSPFTFGQGTKLEIK |
| SEQ ID NO: 36 | DNA VL | GAGAATGTGCTGACCCAGTCTCCTGCCACACTGTCTGTT |
| | | AGCCCTGGCGAGAGAGCTACCCTGAGCTGCAGAGCCTCT |
| | | TCCTCCGTGAACTACATCTACTGGTATCAGCAGAAGCCC |
| | | GGCCAGGCTCCTAGACTGCTGATCTACTACACCTCCAAT |
| | | CTGGCCCCTGGCATCCCTGCCAGATTTTCCGGATCTGGCT |
| | | CCGGCAACGAGTATACCCTGACCATCTCCAGCCTGCAGT |
| | | CCGAGGACTTTGCTGTGTACTATTGCCAGCAGTTCACAA |
| | | GCAGCCCTTTCACCTTTGGCCAGGGCACCAAGCTGGAAA |
| | | TCAAA |

Antibody B-H.IG LC-4

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3 (Combined) | QQFTSSPFT |
| SEQ ID NO: 29 | VL | QNVLTQPPSASGTPGQRVTISCRASSSVNYIYWYQQLPGTA |
| | | PKLLIYYTSNLAPGVPDRFSGSGSGNSYSLAISGLRSEDEAD |
| | | YYCQQFTSSPFTFGTGTKVTVL |
| SEQ ID NO: 37 | DNA VL | CAGAATGTGCTGACCCAACCTCCTTCCGCCTCTGGCACA |
| | | CCTGGACAGAGAGTGACAATCTCCTGCCGGGCCTCCTCC |
| | | TCCGTGAACTACATCTACTGGTATCAGCAGCTGCCCGGC |
| | | ACCGCTCCTAAACTGCTGATCTACTACACCTCCAATCTG |
| | | GCCCCTGGCGTGCCCGATAGATTTTCCGGATCTGGCTCC |
| | | GGCAACTCCTACAGCCTGGCTATCTCTGGCCTGAGATCT |
| | | GAGGACGAGGCCGACTACTACTGCCAGCAGTTCACCTCC |
| | | TCTCCATTCACCTTTGGCACCGGCACCAAAGTGACAGTT |
| | | CTT |

Antibody B-H. 1H LC-5

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3 (Combined) | QQFTSSPFT |
| SEQ ID NO: 30 | VL | SNELTQPPSVSVSPGQTARITCRASSSVNYIYWYQQKSGQA |
| | | PVLVIYYTSNLAPGIPERFSGSGSGNMYTLTISGAQVEDEAD |
| | | YYCQQFTSSPFTFGTGTKVTVL |
| SEQ ID NO: 38 | DNA VL | TCTAATGAGCTGACCCAGCCTCCTTCCGTGTCCGTGTCTC |
| | | CTGGACAGACCGCCAGAATTACCTGCCGGGCCTCCTCCT |
| | | CCGTGAACTACATCTACTGGTATCAGCAGAAGTCCGGCC |
| | | AGGCTCCTGTGCTCGTGATCTACTACACCTCCAATCTGGC |
| | | CCCTGGCATCCCTGAGAGATTCTCCGGATCTGGCTCCGG |
| | | CAACATGTACACCCTGACCATCTCTGGCGCCCAGGTGGA |
| | | AGATGAGGCCGACTACTACTGCCAGCAGTTCACCTCCTC |
| | | TCCATTCACCTTTGGCACCGGCACCAAAGTGACAGTTCT |
| | | T |

Antibody B-H.1

| | | |
|---|---|---|
| SEQ ID NO: | Chain 1: Fc only | METDTLLLWVLLLWVPGSTGDKTHTCPPCPAPELLGGPSV |
| 3280 | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPCREEMTK |
| | | NQVSLWCLVKGFYPSDIAVEWESNGQPENNYKTTPPVLDS |
| | | DGSFFLYSKLTVDKSRWQQGNVFSCSVMHEALHNRFTQKS |
| | | LSLSPGK |
| SEQ ID NO: | Chain 2: humanized B- | METDTLLLWVLLLWVPGSTGEVQLVESGGGLVQPGGSLRL |
| 3281 | H scFv | SCAASGFTFSNFGMHWVRQAPGKGLEWVSYISSGSSTIYYA |
| | | DTLKGRFTISRDNSKNTLYLQMNSLRAEDTAVYYCARRGE |
| | | GAMDYWGQGTTVTVSSGGGGSGGGGSGGGGSGGGGSDN |
| | | QLTQSPSFLSASVGDRVTITCRASSSVNYIYWYQQKPGKAP |
| | | KLLIYYTSNLAPGVPSRFSGSGSGNEYTLTISSLQPEDFATY |
| | | YCQQFTSSPFTFGQGTKLEIKGGGGSDKTHTCPPCPAPELLG |
| | | GPSVFLFPPKPKDTLMISRTPEVTCVVVDVSHEDPEVKFNW |
| | | YVDGVEVHNAKTKPREEQYNSTYRVVSVLTVLHQDWLNG |
| | | KEYKCKVSNKALPAPIEKTISKAKGQPREPQVCTLPPSREE |
| | | MTKNQVSLSCAVKGFYPSDIAVEWESNGQPENNYKTTPPV |
| | | LDSDGSFFLVSKLTVDKSRWQQGNVFSCSVMHEALHNHYT |
| | | QKSLSLSPGKGGGGSGGGGSGLNDIFEAQKIEWHE |
| SEQ ID NO: | scFv | EVQLVESGGGLVQPGGSLRLSCAASGFTFSNFGMHWVRQA |
| 3642 | | PGKGLEWVSYISSGSSTIYYADTLKGRFTISRDNSKNTLYLQ |
| | | MNSLRAEDTAVYYCARRGEGAMDYWGQGTTVTVSSGGG |
| | | GSGGGGSGGGGSGGGGSDNQLTQSPSFLSASVGDRVTITCR |
| | | ASSSVNYIYWYQQKPGKAPKLLIYYTSNLAPGVPSRFSGSG |
| | | SGNEYTLTISSLQPEDFATYYCQQFTSSPFTFGQGTKLEIK |

Antibody B-H.2

| | | |
|---|---|---|
| SEQ ID NO: | ScFv | EVQLVESGGGLVQPGGSLRLSCAASGFTFSNFGMHWVRQA |
| 1338 | | PGKGLEWVSYISSGSSTIYYADTLKGRFTISRDNSKNTLYLQ |
| | | MNSLRAEDTAVYYCARRGEGAMDYWGQGTTVTVSSGGG |
| | | GSGGGGSGGGGSGGGGSDNQLTQSPSSLSASVGDRVTITCR |
| | | ASSSVNYIYWYQQKPGKAPKLLIYYTSNLAPGVPSRFSGSG |
| | | SGNDYTLTISSLQPEDFATYYCQQFTSSPFTFGQGTKLEIK |

Antibody B-H.3

| | | |
|---|---|---|
| SEQ ID NO: | scFv | EVQLVESGGGLVQPGGSLRLSCAASGFTFSNFGMHWVRQA |
| 1339 | | PGKGLEWVSYISSGSSTIYYADTLKGRFTISRDNSKNTLYLQ |
| | | MNSLRAEDTAVYYCARRGEGAMDYWGQGTTVTVSSGGG |
| | | GSGGGGSGGGGSGGGGSSNELTQPPSVSVSPGQTARITCRA |
| | | SSSVNYIYWYQQKSGQAPVLVIYYTSNLAPGIPERFSGSGSG |
| | | NMYTLTISGAQVEDEADYYCQQFTSSPFTFGTGTKVTVL |

Antibody B-H.4

| | | |
|---|---|---|
| SEQ ID NO: | scFv | QVQLVESGGGVVQPGRSLRLSCAASGFTFSNFGMHWVRQ |
| 1340 | | APGKGLEWVAYISSGSSTIYYADTLKGRFTISRDNSKNTLY |
| | | LQMNSLRAEDTAVYYCARRGEGAMDYWGQGTTVTVSSG |
| | | GGGSGGGGSGGGGSGGGGSDNQLTQSPSFLSASVGDRVTI |
| | | TCRASSSVNYIYWYQQKPGKAPKLLIYYTSNLAPGVPSRFS |
| | | GSGSGNEYTLTISSLQPEDFATYYCQQFTSSPFTFGQGTKLEI |
| | | K |

Antibody B-H.5

| | | |
|---|---|---|
| SEQ ID NO: | scFv | QVQLVESGGGVVQPGRSLRLSCAASGFTFSNFGMHWVRQ |
| 1341 | | APGKGLEWVAYISSGSSTIYYADTLKGRFTISRDNSKNTLY |
| | | LQMNSLRAEDTAVYYCARRGEGAMDYWGQGTTVTVSSG |
| | | GGGSGGGGSGGGGSGGGGSDNQLTQSPSSLSASVGDRVTI |
| | | TCRASSSVNYIYWYQQKPGKAPKLLIYYTSNLAPGVPSRFS |
| | | GSGSGNDYTLTISSLQPEDFATYYCQQFTSSPFTFGQGTKLE |
| | | IK |

Antibody B-H.6

| | | |
|---|---|---|
| SEQ ID NO: | scFv | QVQLVESGGGVVQPGRSLRLSCAASGFTFSNFGMHWVRQ |
| 1342 | | APGKGLEWVAYISSGSSTIYYADTLKGRFTISRDNSKNTLY |
| | | LQMNSLRAEDTAVYYCARRGEGAMDYWGQGTTVTVSSG |
| | | GGGSGGGGSGGGGSGGGGSSNELTQPPSVSVSPGQTARITC |
| | | RASSSVNYIYWYQQKSGQAPVLVIYYTSNLAPGIPERFSGS |
| | | GSGNMYTLTISGAQVEDEADYYCQQFTSSPFTFGTGTKVTV |

TABLE 3
Constant region amino acid sequences of human IgG heavy chains
and human kappa light chain.

| | | |
|---|---|---|
| Human kappa | LC | RTVAAPSVFI FPPSDEQLKS GTASVVCLLN NFYPREAKVQ WKVDNALQSG |
| constant region | | NSQESVTEQD SKDSTYSLSS TLTLSKADYE KHKVYACEVT HQGLSSPVTK |
| SEQ ID NO: 39 | | SFNRGEC |
| Human | LC | RTVAAPSVFIFPPSDEQLKSGTASVVCLLNNFYPREAKVQWKVDNALQSGNSQ |
| Immunoglobulin | | ESVTEQDSKDSTYSLSSTLTLSKADYEKHKVYACEVTHQGLSSPVTKSFNRGE |
| kappa constant C | | C |
| region | | |
| SEQ ID NO: 3644 | | |
| IgG4 (S228P) | HC | ASTKGPSVFPLAPCSRSTSESTAALGCLVKDYFPEPVTVSWNSGALTSGVHTFP |
| mutant constant | | AVLQSSGLYSLSSVVTVPSSSLGTKTYTCNVDHKPSNTKVDKRVESKYGPPCPP |
| region (EU | | CPAPEFLGGPSVFLFPPKPKDTLMISRTPEVTCVVVDVSQEDPEVQFNWYVDG |
| Numbering) | | VEVHNAKTKPREEQFNSTYRVVSVLTVLHQDWLNGKEYKCKVSNKGLPSSIE |
| SEQ ID NO: 40 | | KTISKAKGQPREPQVYTLPPSQEEMTKNQVSLTCLVKGFYPSDIAVEWESNGQ |
| | | PENNYKTTPPVLDSDGSFFLYSRLTVDKSRWQEGNVFSCSVMHEALHNHYTQ |
| | | KSLSLSLG |
| IgG1 wild type | HC | ASTKGPSVFPLAPSSKSTSGGTAALGCLVKDYFPEPVTVSWNSGALTSGVHTFP |
| SEQ ID NO: 41 | | AVLQSSGLYSLSSVVTVPSSSLGTQTYICNVNHKPSNTKVDKRVEPKSCDKTHT |
| | | CPPCPAPELLGGPSVFLFPPKPKDTLMISRTPEVTCVVVDVSHEDPEVKFNWYV |
| | | DGVEVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIAVEWESN |
| | | GQPENNYKTTPPVLDSDGSFFLYSKLTVDKSRWQQGNVFSCSVMHEALHNHY |
| | | TQKSLSLSPGK |
| IgG1 (N297A) | HC | ASTKGPSVFPLAPSSKSTSGGTAALGCLVKDYFPEPVTVSWNSGALTSGVHTFP |
| mutant constant | | AVLQSSGLYSLSSVVTVPSSSLGTQTYICNVNHKPSNTKVDKRVEPKSCDKTHT |
| region (EU | | CPPCPAPELLGGPSVFLFPPKPKDTLMISRTPEVTCVVVDVSHEDPEVKFNWYV |
| Numbering) | | DGVEVHNAKTKPREEQYASTYRVVSVLTVLHQDWLNGKEYKCKVSNKALPA |
| SEQ ID NO: 42 | | PIEKTISKAKGQPREPQVYTLPPSREEMTKNQVSLTCLVKGFYPSDIAVEWESN |
| | | GQPENNYKTTPPVLDSDGSFFLYSKLTVDKSRWQQGNVFSCSVMHEALHNHY |
| | | TQKSLSLSPGK |
| IgM constant | HC | GSASAPTLFPLVSCENSPSDTSSVAVGCLAQDFLPDSITFSWKYKNNSDISSTRG |
| delta CDC | | FPSVLRGGKYAATSQVLLPSKDVMQGTDEHVVCKVQHPNGNKEKNVPLPVIA |
| (P311A, P313S) | | ELPPKVSVFVPPRDGFFGNPRKSKLICQATGFSPRQIQVSWLREGKQVGSGVTT |
| SEQ ID NO: 73 | | DQVQAEAKESGPTTYKVTSTLTIKESDWLGQSMFTCRVDHRGLTFQQNASSM |
| | | CVPDQDTAIRVFAIPPSFASIFLTKSTKLTCLVTDLTTYDSVTISWTRQNGEAVK |
| | | THTNISESHPNATFSAVGEASICEDDWNSGERFTCTVTHTDLASSLKQTISRPKG |
| | | VALHRPDVYLLPPAREQLNLRESATITCLVTGFSPADVFVQWMQRGQPLSPEK |
| | | YVTSAPMPEPQAPGRYFAHSILTVSEEEWNTGETYTCVVAHEALPNRVTERTV |
| | | DKSTGKPTLYNVSLVMSDTAGTCY |
| IgGA1 | HC | ASPTSPKVFPLSLCSTQPDGNVVIACLVQGFFPQEPLSVTWSESGQGVTARNFPP |
| SEQ ID NO: 74 | | SQDASGDLYTTSSQLTLPATQCLAGKSVTCHVKHYTNPSQDVTVPCPVPSTPPT |
| | | PSPSTPPTPSPSCCHPRLSLHRPALEDLLLGSEANLTCTLTGLRDASGVTFTWTP |
| | | SSGKSAVQGPPERDLCGCYSVSSVLPGCAEPWNHGKTFTCTAAYPESKTPLTA |
| | | TLSKSGNTFRPEVHLLPPPSEELALNELVTLTCLARGFSPKDVLVRWLQGSQEL |
| | | PREKYLTWASRQEPSQGTTTFAVTSILRVAAEDWKKGDTFSCMVGHEALPLAF |
| | | TQKTIDRLAGKPTHVNVSVVMAEVDGTCY |
| IgGA2 | HC | ASPTSPKVFPLSLDSTPQDGNVVVACLVQGFFPQEPLSVTWSESGQNVTARNFP |
| SEQ ID NO: 75 | | PSQDASGDLYTTSSQLTLPATQCPDGKSVTCHVKHYTNSSQDVTVPCRVPPPPP |
| | | CCHPRLSLHRPALEDLLLGSEANLTCTLTGLRDASGATFTWTPSSGKSAVQGPP |
| | | ERDLCGCYSVSSVLPGCAQPWNHGETFTCTAAHPELKTPLTANITKSGNTFRPE |
| | | VHLLPPPSEELALNELVTLTCLARGFSPKDVLVRWLQGSQELPREKYLTWASR |
| | | QEPSQGTTTYAVTSILRVAAEDWKKGETFSCMVGHEALPLAFTQKTIDRMAG |
| | | KPTHINVSVVMAEADGTCY |
| Human Ig_J chain | HC | MKNHLLFWGVLAVFIKAVHVKAQEDERIVLVDNKCKCARITSRIIRSSEDPNED |
| SEQ ID NO: 76 | | IVERNIRIIVPLNNRENISDPTSPLRTRFVYHLSDLCKKCDPTEVELDNQIVTA |
| | | TQSNICDEDSATETCYTYDRNKCYTAVVPLVYGGETKMVETALTPDACYPD |
| Human | HC | ASTKGPSVFPLAPSSKSTSGGTAALGCLVKDYFPEPVTVSWNSGALTSGVHTFP |
| Immunoglobulin | | AVLQSSGLYSLSSVVTVPSSSLGTQTYICNVNHKPSNTKVDKKVEPKSCDKTHT |
| heavy chain | | CPPCPAPELLGGPSVFLFPPKPKDTLMISRTPEVTCVVVDVSHEDPEVKFNWYV |
| SEQ ID NO: 3645 | | DGVEVHNAKTKPREEQYASTYRVVSVLTVLHQDWLNGKEYKCKVSNKALPA |
| | | PIEKTISKAKGQPREPQVYTLPPSREEMTKNQVSLTCLVKGFYPSDIAVEWESN |
| | | GQPENNYKTTPPVLDSDGSFFLYSKLTVDKSRWQQGNVFSCSVMHEALHNHY |
| | | TQKSLSLSPGK |
| Human | HC | ASTKGPSVFPLAPSSKSTSGGTAALGCLVKDYFPEPVTVSWNSGALTSGVHTFP |
| Immunoglobulin | | AVLQSSGLYSLSSVVTVPSSSLGTQTYICNVNHKPSNTKVDKKVEPKSCDKTHT |
| heavy chain | | CPPCPAPELLGGPSVFLFPPKPKDTLMISRTPEVTCVVVDVSHEDPEVKFNWYV |
| SEQ ID NO: 3646 | | DGVEVHNAKTKPREEQYASTYRVVSVLTVLHQDWLNGKEYKCKVSNKALPA |
| | | PIEKTISKAKGQPREPQVYTLPPSREEMTKNQVSLTCLVKGFYPSDIAVEWESN |
| | | GQPENNYKTTPPVLDSDGSFFLYSKLTVDKSRWQQGNVFSCSVMHEALHNHY |
| | | TQKSLSLSPGKEPEA |
| Human | HC | ASTKGPSVFPLAPSSKSTSGGTAALGCLVKDYFPEPVTVSWNSGALTSGVHTFP |
| Immunoglobulin | | AVLQSSGLYSLSSVVTVPSSSLGTQTYICNVNHKPSNTKVDKRVEPKSCDKTHT |
| heavy chain | | CPPCPAPELLGGPSVFLFPPKPKDTLMISRTPEVTCVVVDVSHEDPEVKFNWYV |
| SEQ ID NO: 3647 | | DGVEVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIAVEWESN |
| | | GQPENNYKTTPPVLDSDGSFFLYSKLTVDKSRWQQGNVFSCSVMHEALHNHY |
| | | TQKSLSLSPGK |
| Human Fc Knob | HC | DKTHTCPPCPAPELLGGPSVFLFPPKPKDTLMISRTPEVTCVVVDVSHEDPEVK |
| cys N297A | | FNWYVDGVEVHNAKTKPREEQYASTYRVVSVLTVLHQDWLNGKEYKCKVS |
| SEQ ID NO: 3648 | | NKALPAPIEKTISKAKGQPREPQVYTLPPCREEMTKNQVSLWCLVKGFYPSDIA |
| | | VEWESNGQPENNYKTTPPVLDSDGSFFLYSKITVDKSRWQQGNVFSCSVMHE |
| | | ALHNHYTQKSLSLSPGK. |
| Human IgG1 hole | HC | ASTKGPSVFPLAPSSKSTSGGTAALGCLVKDYFPEPVTVSWNSGALTSGVHTFP |
| cys N297A | | AVLQSSGLYSLSSVVTVPSSSLGTQTYICNVNHKPSNTKVDKRVEPKSCDKTHT |
| SEQ ID NO: 3649 | | CPPCPAPELLGGPSVFLFPPKPKDTLMISRTPEVTCVVVDVSHEDPEVKFNWYV |
| | | DGVEVHNAKTKPREEQYASTYRVVSVLTVLHQDWLNGKEYKCKVSNKALPA |
| | | PIEKTISKAKGQPREPQVCTLPPSREEMTKNQVSLSCAVKGFYPSDIAVEWESN |
| | | GQPENNYKTTPPVLDSDGSFFLVSKLTVDKSRWQQGNVFSCSVMHEALHNHY |
| | | TQKSLSLSPGK |

TABLE 4
Exemplary Fc KiH mutations and optional Cysteine mutations

| Position | Knob Mutation | Hole Mutation |
|---|---|---|
| T366 | T366W | T366S |
| L368 | — | L368A |
| Y407 | — | Y407V |

Additional Cysteine Mutations to form a stabilizing disulfide bridge

| Position | Knob CH3 | Hole CH3 |
|---|---|---|
| S354 | S354C | — |
| Y349 | — | Y349C |

TABLE 5
CRS grading

| | |
|---|---|
| Gr1 | Supportive care only |
| Gr2 | IV therapies +/− hospitalization. |
| Gr3 | Hypotension requiring IV fluids or low-dose vasoactives or |
| | hypoxemia requiring oxygen, CPAP, or BIPAP. |
| Gr4 | Hypotension requiring high-dose vasoactives or hypoxemia |
| | requiring mechanical ventilation. |
| Gr 5 | Death |

TABLE 6
CTCAE v 4.0 CRS grading scale

| CRS | |
|---|---|
| grade | Characteristics |
| Grade 1 | Mild; No infusion interruption; No intervention |
| Grade 2 | Infusion interruption indicated but responds promptly to |
| | symptomatic treatment (e.g., antihistamines, NSAIDS, narcotics, |
| | IV fluids); prophylactic medications indicated for <=24 hrs |
| Grade 3 | Prolonged (e.g., not rapidly responsive to symptomatic |
| | medications and/or brief interruption of infusion); recurrence |
| | of symptoms following initial improvement; hospitalization |
| | indicated for clinical sequelae (e.g., renal impairment, |
| | pulmonary infiltrates) |
| Grade 4 | Life threatening consequences; pressor or ventilator support |

TABLE 7
NCI CRS grading scale

| CRS | |
|---|---|
| grade | Characteristics |
| Grade 1 | Symptoms are not life threatening and require symptomatic |
| | treatment only; e.g., fever, nausea, fatigue, headache, |
| | myalgias, malaise |
| Grade 2 | Symptoms require and respond to moderate intervention; |
| | Oxygen requirement <40% or hypotension responsive to fluids |
| | or low dose pressors or Grade 2 organ toxicity |
| Grade 3 | Symptoms require and respond to aggressive intervention; |
| | Oxygen requirement >=40% or Hypotension requiring high dose |
| | or multiple pressors or grade 3 organ toxicity or grade |
| | 4 transaminitis |
| Grade 4 | Life threatening symptoms Requirement for ventilator support or |
| | Grade 4; organ toxicity (excluding transaminitis) |

TABLE 8
List of TCRβV subfamilies and subfamily members

| Subfamily | Subfamily members |
|---|---|
| TCRβ V6 | TCRβ V6-4*01, TCRβ V6-4*02, TCRβ V6-9*01, |
| Also referred | TCRβ V6-8*01, TCRβ V6-5*01, TCRβ V6-6*02, |
| to as: | TCRβ V6-6*01, TCRβ V6-2*01, TCRβ V6-3*01 |
| TCR VB 13.1 | or TCRβ V6-1*01. |
| TCRβ V10 | TCRβ V10-1*01, TCRβ V10-1*02, TCRβ V10-3*01 |
| Also referred | or TCRβ V10-2*01 |
| to as: | |
| TCRβ V12 | |
| TCRβ V12 | TCRβ V12-4*01, TCRβ V12-3*01, or TCRβ V12-5*01 |
| Also referred | |
| to as: | |
| TCRβ V8.1 | |
| TCRβ V5 | TCRβ V5-5*01, TCRβ V5-6*01, TCRβ V5-4*01, |
| | TCRβ V5-8*01, TCRβ V5-1*01 |
| TCRβ V7 | TCRβ V7-7*01, TCRβ V7-6*01, TCRβ V7 -8*02, |
| | TCRβ V7 -4*01, TCRβ V7-2*02, TCRβ V7-2*03, |
| | TCRβ V7-2*01, TCRβ V7-3*01, TCRβ V7-9*03, |
| | or TCRβ V7-9*01 |
| TCRβ V15 | TCRβ V15*01 |
| TCRβ V30 | TCRβ V30*01, or TCRβ V30*02 |
| TCRβ V19 | TCRβ V19*01, or TCRβ V19*02 |
| TCRβ V27 | TCRβ V27*01. |
| TCRβ V28 | TCRβ V28*01. |
| TCRβ V20 | TCRβ V20-1*01, or TCRβ V20-1*02 |

TABLE 10A
Exemplary TCRβV antibody molecules

| TRBV | TRBV | Reagents monoclonal antibodies | | |
|---|---|---|---|---|
| gene name | allele name | Clone name and Specificity | Company product | Isotype |
| TRBV5-1 | TRBV5-1*01 | IMMU157 (TRBV5-1) | Serotec Vbeta5.1 | Mouse IgG2a |
| | | | Coulter Vbeta5.1 | |
| | TRBVS-1*02 | LC4 (TRBV5-1) | Pierce Endogen V beta 5(c) | Mouse IgG1 |
| | | | BD Biosciences Vbeta5(c) | |
| TRBV5-4 | TRBV5-4*01 | | | |
| | TRBV5-4*02 | | | |
| | TRBV5-4*03 | | | |
| | TRBV5-4*04 | | | |
| TRBV5-5 | TRBVS-5*01 | 3D11 (TRBV5-5) | Serotec VBETA5.3 | Mouse IgG1 |
| | | 1C1 (TRBV5-5, TRBV5-6) | Coulter Vbeta5.3 | |
| | TRBV5-5*02 | W112 (TRBV5-5) | Pierce Endogen V beta 5(a) | Mouse IgG1 |
| | | MH3-2 (TRBV5-5, | BD Biosciences Vbeta5(a) | |
| | TRBV5-5*03 | TRBV5-6) | Pierce Endogen V beta 5(b) | Mouse IgG1 |
| | | | Serotec V beta 5.2/5.3 | |
| | | | BD Biosciences Vbeta5(b) | |
| | | | BD Biosciences Vbeta5 | Mouse IgG2a |
| | | 4H11 (TM27) as disclosed | | |
| | | in U.S. Pat. No. 5,861,155 | | |
| TRBV5-6 | TRBV5-6*01 | 36213 (TRBV5-6) | Serotec Vbeta5.2 | Mouse IgG1 |
| | | 1C1 (TRBV5-5, TRBV5-6) | BD Biosciences Vbeta5(a) | Mouse IgG1 |
| | | MH3-2 (TRBV5-5, | BD Biosciences Vbeta5 | Mouse IgG2a |
| | | TRBV5-6) | | |
| TRBV5-8 | TRBV5-8*01 | | | |
| | TRBV5-8*02 | | | |
| TRBV6-1 | TRBV6-1*01 | BAM13 (TRBV6-1, | Pierce Endogen V beta 13 | Mouse IgG1 |
| | | TRBV6-5) | BD Biosciences Vbeta13.1, | |
| | | | 13.3 | |
| TRBV6-2 | TRBV6-2*01 | H132 | Coulter Vbeta 13.2 | Mouse IgG1 |
| TRBV6-3 | TRBV6-3*01 | | | |
| TRBV6-4 | TRBV6-4*01 | | | |
| | TRBV6-4*02 | | | |
| TRBV6-5 | TRBV6-5*01 | IMMU 222 (TRBV6-5, | Serotec V BETA 13.1 | Mouse IgG2b |
| | | TRBV6-6 and TRBV6-9) | Coulter Vbeta13.1 | |
| | | BAM13 (TRBV6-1, | Pierce Endogen V beta 13 | Mouse IgG1 |
| | | TRBV6-5) | BD Biosciences Vbeta13.1, | |
| | | | 13.3 | |
| TRBV6-6 | TRBV6-6*01 | JU-74 (TRBV6-6) | Serotec Vbeta13.6 | Mouse IgG1 |
| | TRBV6-6*02 | JU74.3 (TRBV6-6) | Coulter Vbeta13.6 | |
| | TRBV6-6*03 | IMMU 222 (TRBV6-5, | Serotec V BETA 13.1 | Mouse IgG2b |
| | TRBV6-6*04 | TRBV6-6 and TRBV6-9) | Coulter Vbeta13.1 | |
| | TRBV6-6*05 | | | |
| TRBV6-8 | TRBV6-8*01 | | | |
| TRBV6-9 | TRBV6-9*01 | IMMU 222 (TRBV6-5, | Serotec V BETA 13.1 | Mouse IgG2b |
| | | TRBV6-6 and TRBV6-9) | Coulter Vbeta13.1 | |
| TRBV7-2 | TRBV7-2*01 | OT145 (TRBV7-2) | Pierce Endogen V beta 6.7 | Mouse IgG1 |
| | TRBV7-2*02 | | BD Biosciences Vbeta6.7 | |
| | TRBV7-2*03 | | | |
| | TRBV7-2*04 | | | |
| TRBV7-3 | TRBV7-3*01 | | | |
| | TRBV7-3*04 | | | |
| | TRBV7-3*05 | | | |
| TRBV7-4 | TRBV7-4*01 | | | |
| TRBV7-6 | TRBV7-6*01 | | | |
| | TRBV7-6*02 | | | |
| TRBV7-7 | TRBV7-7*01 | | | |
| | TRBV7-7*02 | | | |
| TRBV7-8 | TRBV7-8*01 | | | |
| | TRBV7-8*02 | | | |
| | TRBV7-8*03 | | | |
| TRBV7-9 | TRBV7-9*01 | | | |
| | TRBV7-9*02 | | | |
| | TRBV7-9*03 | | | |
| | TRVB7-9*04 | | | |
| | TRBV7-9*05 | | | |
| | TRBV7-9*06 | | | |
| | TRBV7-9*07 | | | |
| TRBV10-1 | TRBV10-1*01 | S511 (TRBV10-1, | Pierce Endogen V beta 12 | Mouse IgG2b |
| | TRBV10-1*02 | TRBV10-2, TRBV10-3) | BD Biosciences Vbeta12 | |
| TRBV10-2 | TRBV10-2*01 | | | |
| | TRBV10-2*02 | | | |
| TRBV10-3 | TRBV10-3*01 | VER2.32.1 (TRBV10-3) | Serotec Vbeta12 | Mouse IgG2a |
| | TRBV10-3*02 | S511 (TRBV10-1, | Coulter Vbeta12 | |
| | TRBV10-3*03 | TRBV10-2, TRBV10-3) | Pierce Endogen V beta 12 | Mouse IgG2b |
| | TRBV10-3*04 | | BD Biosciences Vbeta12 | |
| TRBV12-3 | TRBV12-3*01 | 56C5 (TRBV12-3, | Serotec Vbeta8.1/8.2 | Mouse IgG2a |
| | | TRBV12-4) | Coulter Vbeta8 | |
| TRBV12-4 | TRBV12-4*01 | 56C5.2 (TRBV12-3, | Pierce Endogen V beta 8(a) | Mouse IgG2b |
| | | TRBV12-4) | BD Biosciences Vbeta8 | |
| | TRBV12-4*02 | 16G8 (TRBV12-3, | Pierce Endogen V beta 8(b) | Mouse IgG2a |
| | | TRBV12-4) | BD Biosciences | Mouse IgG2b |
| | | MX-6 (TRBV12-3, | Vbeta8 | |
| | | TRBV12-4) | | |
| | | JR2 (TRBV12-3, TRBV12- | | |
| | | 4, TRBV12-5) | | |
| TRBV12-5 | TRBV12-5*01 | JR2 (TRBV12-3, TRBV12- | BD Biosciences Vbeta8 | Mouse IgG2b |
| | | 4, TRBV12-5) | | |
| TRBV15 | TRBV15*01 | | | |
| | TRBV15*02 | | | |
| | TRBV15*03 | | | |
| TRBV19 | TRBV19*01 | C1 (TRBV19) | Pierce Endogen V beta 17 | Mouse IgG1 |
| | | E17.5F3 (TRBV19) | BD Biosciences Vbeta17 | |
| | TRBV19*02 | E17.5F3.15.13 (TRBV19) | Serotec Vbeta17 | Mouse IgG1 |
| | TRBV19*03 | | Coulter Vbeta17 | |
| TRBV20-1 | TRBV20-1*01 | MPB2D5 (TRBV20-1) | Serotec VBETA2 | Mouse IgG1 |
| | TRBV20-1*02 | | Coulter Vbeta2 | |
| | TRBV20-1*03 | | | |
| | TRBV20-1*04 | | | |
| | TRBV20-1*05 | | | |
| | TRBV20-1*06 | | | |
| | TRBV20-1*07 | | | |
| TRBV28 | TRBV28*01 | CH92 (TRBV28) | Serotec Vbeta3 | Mouse IgM |
| | | 8F10 (TRBV28) | Coulter Vbeta3 | |
| | | JOVI-3 (TRBV28) | Pierce Endogen V beta 3.1 | Mouse IgG1 |
| | | | BD Biosciences Vbeta3 | Mouse IgG2a |
| | TRBV30*02 | | | |
| | TRBV30*04 | | | |
| | TRBV30*05 | | | |

TABLE 10B
Amino acid sequences for anti TCRβ V5 antibodies. Amino acid and nucleotide sequences for
murine and humanized antibody molecules which bind to TCRVB 5 (e.g., TCRVB 5-5 or TCRVB 5-6).
The amino acid of the heavy and light chain CDRs, and the amino acid
and nucleotide sequences of the heavy and light chain
variable regions, and the heavy and light chains are shown.
Murine antibody C, also referred to as 4H11

| | | |
|---|---|---|
| SEQ ID NO: 1315 | HC CDR1 (Kabat) | AYGVN |
| SEQ ID NO: 1316 | HC CDR2 (Kabat) | MIWGDGNTDYNSALKS |
| SEQ ID NO: 1317 | HC CDR3 (Kabat) | DRVTATLYAMDY |
| SEQ ID NO: 1318 | HC CDR1 (Chothia) | GFSLTAY |
| SEQ ID NO: 1319 | HC CDR2 (Chothia) | WGDGN |
| SEQ ID NO: 1317 | HC CDR3 (Chothia) | DRVTATLYAMDY |
| SEQ ID NO: 1320 | HC CDR1 (Combined) | GFSLTAYGVN |
| SEQ ID NO: 1316 | HC CDR2 (Combined) | MIWGDGNTDYNSALKS |
| SEQ ID NO: 1317 | HC CDR3 (Combined) | DRVTATLYAMDY |
| SEQ ID NO: 1321 | LC CDR1 (Kabat) | SASQGISNYLN |
| SEQ ID NO: 1322 | LC CDR2 (Kabat) | YTSSLHS |
| SEQ ID NO: 1323 | LC CDR3 (Kabat) | QQYSKLPRT |
| SEQ ID NO: 1321 | LC CDR1 (Chothia) | SASQGISNYLN |
| SEQ ID NO: 1322 | LC CDR2 (Chothia) | YTSSLHS |
| SEQ ID NO: 1323 | LC CDR3 (Chothia) | QQYSKLPRT |
| SEQ ID NO: 1321 | LC CDR1 (Combined) | SASQGISNYLN |
| SEQ ID NO: 1322 | LC CDR2 (Combined) | YTSSLHS |
| SEQ ID NO: 1323 | LC CDR3(Combined) | QQYSKLPRT |
| SEQ ID NO: 232 | VH | DIQMTQTTSSLSASLGDRVTISCSASQGISNYLNWYQQK |
| | | PDGTVKLLIYYTSSLHSGVPSRFSGSGSGTDYSLTISNL |
| | | EPEDIATYYCQQYSKLPRTFGGGTKVEIK |
| SEQ ID NO: 233 | VL | QVQLKESGPGLVAPSQSLSITCTVSGFSLTAYGVNWVR |
| | | QPPGKGLEWLGMIWGDGNTDYNSALKSRLSISKDNSKS |
| | | QVFLKMNSLQTDDTARYYCARDRVTATLYAMDYWGQ |
| | | GTSVTVSS |

Humanized antibody C
C-H-1 antibody

| | | |
|---|---|---|
| SEQ ID NO: 1315 | HC CDR1 (Kabat) | AYGVN |
| SEQ ID NO: 1316 | HC CDR2 (Kabat) | MIWGDGNTDYNSALKS |
| SEQ ID NO: 1317 | HC CDR3 (Kabat) | DRVTATLYAMDY |
| SEQ ID NO: 1318 | HC CDR1 (Chothia) | GESLTAY |
| SEQ ID NO: 1319 | HC CDR2 (Chothia) | WGDGN |
| SEQ ID NO: 1317 | HC CDR3 (Chothia) | DRVTATLYAMDY |
| SEQ ID NO: 1320 | HC CDR1 (Combined) | GFSLTAYGVN |
| SEQ ID NO: 1316 | HC CDR2 (Combined) | MIWGDGNTDYNSALKS |
| SEQ ID NO: 1317 | HC CDR3 (Combined) | DRVTATLYAMDY |
| SEQ ID NO: 1321 | LC CDR1 (Kabat) | SASQGISNYLN |
| SEQ ID NO: 1322 | LC CDR2 (Kabat) | YTSSLHS |
| SEQ ID NO: 1323 | LC CDR3 (Kabat) | QQYSKLPRT |
| SEQ ID NO: 1321 | LC CDR1 (Chothia) | SASQGISNYLN |
| SEQ ID NO: 1322 | LC CDR2 (Chothia) | YTSSLHS |
| SEQ ID NO: 1323 | LC CDR3 (Chothia) | QQYSKLPRT |
| SEQ ID NO: 1321 | LC CDR1 (Combined) | SASQGISNYLN |
| SEQ ID NO: 1322 | LC CDR2 (Combined) | YTSSLHS |
| SEQ ID NO: 1323 | LC CDR3(Combined) | QQYSKLPRT |
| SEQ ID NO: 1324 | VL | DIQMTQSPSSLSASVGDRVTITCSASQGISNYLNWYQQTP |
| | | GKAPKLLIYYTSSLHSGVPSRFSGSGSGTDYTFTISSLQP |
| | | EDIATYYCQQYSKLPRTFGQGTKLQIT |
| SEQ ID NO: 1325 | VH | QVQLQESGPGLVRPSQTLSLTCTVSGFSLTAYGVNWVR |
| | | QPPGRGLEWLGMIWGDGNTDYNSALKSRVTMLKDTSK |
| | | NQFSLRLSSVTAADTAVYYCARDRVTATLYAMDYW |

Humanized antibody C Variable light chain (VL)

| | | | |
|---|---|---|---|
| SEQ ID NO: 3000 | VL | C-H-VL.1 | DIQMTQSPSFLSASVGDRVTITCSASQGISNYLNWYQQK |
| | | | PGKAVKLLIYYTSSLHSGVPSRFSGSGSGTEYTLTISSLQ |
| | | | PEDFATYYCQQYSKLPRTFGGGTKVEIK |
| SEQ ID NO: 3001 | VL | C-H-VL.2 | DIQMTQSPSSLSASVGDRVTITCSASQGISNYLNWYQQK |
| | | | PGKAVKLLIYYTSSLHSGVPSRFSGSGSGTDYTLTISSLQ |
| | | | PEDFATYYCQQYSKLPRTFGGGTKVEIK |
| SEQ ID NO: 3002 | VL | C-H-VL.3 | DIQMTQSPSSLSASVGDRVTITCSASQGISNYLNWYQQK |
| | | | PGKVVKLLIYYTSSLHSGVPSRFSGSGSGTDYTLTISSLQ |
| | | | PEDVATYYCQQYSKLPRTFGGGTKVEIK |
| SEQ ID NO: 3003 | VL | C-H-VL.4 | DIQMTQSPSSLSASVGDRVTITCSASQGISNYLNWYQQK |
| | | | PGQAVKLLIYYTSSLHSGVPSRFSGSGSGTDYTLTISSLQ |
| | | | PEDVATYYCQQYSKLPRTFGGGTKVEIK |
| SEQ ID NO: 3004 | VL | C-H-VL.5 | DIQMTQSPSSLSASVGDRVTITCSASQGISNYLNWYQQK |
| | | | PGKAVKLLIYYTSSLHSGVPSRFSGSGSGTDYTFTISSLQ |
| | | | PEDIATYYCQQYSKLPRTFGGGTKVEIK |
| SEQ ID NO: 3005 | VL | C-H-VL.6 | DIQMTQSPSSLSASVGDRVTITCSASQGISNYLNWYQQK |
| | | | PGKTVKLLIYYTSSLHSGIPSRFSGSGSGTDYTLTIRSLQP |
| | | | EDFATYYCQQYSKLPRTFGGGTKVEIK |
| SEQ ID NO: 3006 | VL | C-H-VL.7 | AIQMTQSPSSLSASVGDRVTITCSASQGISNYLNWYQQK |
| | | | PGKAVKLLIYYTSSLHSGVPSRFSGSGSGTDYTLTISSLQ |
| | | | PEDFATYYCQQYSKLPRTFGGGTKVEIK |
| SEQ ID NO: 3007 | VL | C-H-VL.8 | DIQMTQSPSSVSASVGDRVTITCSASQGISNYLNWYQQK |
| | | | PGKAVKLLIYYTSSLHSGVPSRFSGSGSGTDYTLTISSLQ |
| | | | PEDFATYYCQQYSKLPRTFGGGTKVEIK |
| SEQ ID NO: 3008 | VL | C-H-VL.9 | DIQMTQSPSSLSASVGDRVTITCSASQGISNYLNWYQQK |
| | | | PGKAVKRLIYYTSSLHSGVPSRFSGSGSGTEYTLTISNLQ |
| | | | PEDFATYYCQQYSKLPRTFGGGTKVEIK |
| SEQ ID NO: 3009 | VL | C-H-VL.10 | AIRMTQSPFSLSASVGDRVTITCSASQGISNYLNWYQQK |
| | | | PAKAVKLFIYYTSSLHSGVPSRFSGSGSGTDYTLTISSLQ |
| | | | PEDFATYYCQQYSKLPRTFGGGTKVEIK |
| SEQ ID NO: 3010 | VL | C-H-VL.11 | DIQMTQSPSSLSASVGDRVTITCSASQGISNYLNWYQQK |
| | | | PGKAVKRLIYYTSSLHSGVPSRFSGSGSGTEYTLTISSLQ |
| | | | PEDFATYYCQQYSKLPRTFGGGTKVEIK |
| SEQ ID NO: 3011 | VL | C-H-VL.12 | DIQMTQSPSTLSASVGDRVTITCSASQGISNYLNWYQQK |
| | | | PGKAVKLLIYYTSSLHSGVPSRFSGSGSGTEYTLTISSLQ |
| | | | PDDFATYYCQQYSKLPRTFGGGTKVEIK |
| SEQ ID NO: 3012 | VL | C-H-VL.13 | DIQMTQSPSSLSASVGDRVTITCSASQGISNYLNWYQQK |
| | | | PGKAVKSLIYYTSSLHSGVPSRFSGSGSGTDYTLTISSLQ |
| | | | PEDFATYYCQQYSKLPRTFGGGTKVEIK |
| SEQ ID NO: 3013 | VL | C-H-VL.14 | DIQMTQSPSSLSASVGDRVTITCSASQGISNYLNWYQQK |
| | | | PGKAVKSLIYYTSSLHSGVPSKFSGSGSGTDYTLTISSLQ |
| | | | PEDFATYYCQQYSKLPRTFGGGTKVEIK |
| SEQ ID NO: 3014 | VL | C-H-VL.15 | DIQMTQSPSSLSASVGDRVTITCSASQGISNYLNWYQQK |
| | | | PEKAVKSLIYYTSSLHSGVPSRFSGSGSGTDYTLTISSLQ |
| | | | PEDFATYYCQQYSKLPRTFGGGTKVEIK |
| SEQ ID NO: 3015 | VL | C-H-VL.16 | DIQMTQSPSAMSASVGDRVTITCSASQGISNYLNWYQQ |
| | | | KPGKVVKRLIYYTSSLHSGVPSRFSGSGSGTEYTLTISSL |
| | | | QPEDFATYYCQQYSKLPRTFGGGTKVEIK |
| SEQ ID NO: 3016 | VL | C-H-VL.17 | DIVMTQSPDSLAVSLGERATINCSASQGISNYLNWYQQ |
| | | | KPGQPVKLLIYYTSSLHSGVPDRFSGSGSGTDYTLTISSL |
| | | | QAEDVAVYYCQQYSKLPRTFGGGTKVEIK |
| SEQ ID NO: 3017 | VL | C-H-VL.18 | EIVMTQSPGTLSLSPGERATLSCSASQGISNYLNWYQQK |
| | | | PGQAVKLLIYYTSSLHSGIPDRFSGSGSGTDYTLTISRLEP |
| | | | EDFAVYYCQQYSKLPRTFGGGTKVEIK |
| SEQ ID NO: 3018 | VL | C-H-VL.19 | EIVMTQSPPTLSLSPGERVTLSCSASQGISNYLNWYQQK |
| | | | PGQAVKLLIYYTSSLHSGIPARFSGSGSGTDYTLTISSLQP |
| | | | EDFAVYYCQQYSKLPRTFGGGTKVEIK |
| SEQ ID NO: 3019 | VL | C-H-VL.20 | EIVMTQSPPTLSLSPGERVTLSCSASQGISNYLNWYQQK |
| | | | PGQAVKLLIYYTSSLHSSIPARFSGSGSGTDYTLTISSLQP |
| | | | EDFAVYYCQQYSKLPRTFGGGTKVEIK |
| SEQ ID NO: 3020 | VL | C-H-VL.21 | EIVMTQSPATLSLSPGERATLSCSASQGISNYLNWYQQK |
| | | | PGQAVKLLIYYTSSLHSGIPARFSGSGSGTDYTLTISSLEP |
| | | | EDFAVYYCQQYSKLPRTFGGGTKVEIK |
| SEQ ID NO: 3021 | VL | C-H-VL.22 | EIVMTQSPATLSLSPGERATLSCSASQGISNYLNWYQQK |
| | | | PGQAVKLLIYYTSSLHSGIPARFSGSGSGTDYTLTISRLEP |
| | | | EDFAVYYCQQYSKLPRTFGGGTKVEIK |
| SEQ ID NO: 3022 | VL | C-H-VL.23 | EIVMTQSPATLSLSPGERATLSCSASQGISNYLNWYQQK |
| | | | PGQAVKLLIYYTSSLHSGIPDRFSGSGSGTDYTLTISRLEP |
| | | | EDFAVYYCQQYSKLPRTFGGGTKVEIK |
| SEQ ID NO: 3023 | VL | C-H-VL.24 | EIVMTQSPATLSLSPGERATLSCSASQGISNYLNWYQQK |
| | | | PGLAVKLLIYYTSSLHSGIPDRFSGSGSGTDYTLTISRLEP |
| | | | EDFAVYYCQQYSKLPRTFGGGTKVEIK |
| SEQ ID NO: 3024 | VL | C-H-VL.25 | DIQMIQSPSFLSASVGDRVSIICSASQGISNYLNWYLQKP |
| | | | GKSVKLFIYYTSSLHSGVSSRFSGRGSGTDYTLTIISLKPE |
| | | | DFAAYYCQQYSKLPRTFGGGTKVEIK |
| SEQ ID NO: 3025 | VL | C-H-VL.26 | EIVMTQSPATLSLSPGERATLSCSASQGISNYLNWYQQK |
| | | | PGQAVKLLIYYTSSLHSGIPARFSGSGSGTDYTLTISSLQP |
| | | | EDFAVYYCQQYSKLPRTFGGGTKVEIK |
| SEQ ID NO: 3026 | VL | C-H-VL.27 | EIVMTQSPATLSLSPGERATLSCSASQGISNYLNWYQQK |
| | | | PGQAVKLLIYYTSSLHSGIPARFSGSGPGTDYTLTISSLEP |
| | | | EDFAVYYCQQYSKLPRTFGGGTKVEIK |
| SEQ ID NO: 3027 | VL | C-H-VL.28 | DIVMTQTPLSLSVTPGQPASISCSASQGISNYLNWYLQK |
| | | | PGQSVKLLIYYTSSLHSGVPDRFSGSGSGTDYTLKISRVE |
| | | | AEDVGVYYCQQYSKLPRTFGGGTKVEIK |
| SEQ ID NO: 3028 | VL | C-H-VL.29 | DIVMTQTPLSLSVTPGQPASISCSASQGISNYLNWYLQK |
| | | | PGQPVKLLIYYTSSLHSGVPDRFSGSGSGTDYTLKISRVE |
| | | | AEDVGVYYCQQYSKLPRTFGGGTKVEIK |
| SEQ ID NO: 3029 | VL | C-H-VL.30 | DIVMTQSPAFLSVTPGEKVTITCSASQGISNYLNWYQQK |
| | | | PDQAVKLLIYYTSSLHSGVPSRFSGSGSGTDYTFTISSLE |
| | | | AEDAATYYCQQYSKLPRTFGGGTKVEIK |
| SEQ ID NO: 3030 | VL | C-H-VL.31 | DIVMTQSPLSLPVTPGEPASISCSASQGISNYLNWYLQKP |
| | | | GQSVKLLIYYTSSLHSGVPDRFSGSGSGTDYTLKISRVE |
| | | | AEDVGVYYCQQYSKLPRTFGGGTKVEIK |
| SEQ ID NO: 3031 | VL | C-H-VL.32 | DIVMTQTPLSLPVTPGEPASISCSASQGISNYLNWYLQKP |
| | | | GQSVKLLIYYTSSLHSGVPDRFSGSGSGTDYTLKISRVE |
| | | | AEDVGVYYCQQYSKLPRTFGGGTKVEIK |
| SEQ ID NO: 3032 | VL | C-H-VL.33 | EIVMTQSPATLSVSPGERATLSCSASQGISNYLNWYQQK |
| | | | PGQAVKLLIYYTSSLHSGIPARFSGSGSGTEYTLTISILQS |
| | | | EDFAVYYCQQYSKLPRTFGGGTKVEIK |
| SEQ ID NO: 3033 | VL | C-H-VL.34 | EIVMTQSPATLSVSPGERATLSCSASQGISNYLNWYQQK |
| | | | PGQAVKLLIYYTSSLHSGIPARFSGSGSGTEYTLTISSLQS |
| | | | EDFAVYYCQQYSKLPRTFGGGTKVEIK |
| SEQ ID NO: 3034 | VL | C-H-VL.35 | DIVMTQSPLSLPVTLGQPASISCSASQGISNYLNWYQQR |
| | | | PGQSVKRLIYYTSSLHSGVPDRFSGSGSGTDYTLKISRVE |
| | | | AEDVGVYYCQQYSKLPRTFGGGTKVEIK |
| SEQ ID NO: 3035 | VL | C-H-VL.36 | EITMTQSPAFMSATPGDKVNISCSASQGISNYLNWYQQ |
| | | | KPGEAVKFIIYYTSSLHSGIPPRFSGSGYGTDYTLTINNIE |
| | | | SEDAAYYYCQQYSKLPRTFGGGTKVEIK |
| SEQ ID NO: 3036 | VL | C-H-VL.37 | DIVMTQTPLSSPVTLGQPASISCSASQGISNYLNWYQQR |
| | | | PGQPVKLLIYYTSSLHSGVPDRFSGSGAGTDYTLKISRV |
| | | | EAEDVGVYYCQQYSKLPRTFGGGTKVEIK |
| SEQ ID NO: 3037 | VL | C-H-VL.38 | EIVMTQSPDFQSVTPKEKVTITCSASQGISNYLNWYQQK |
| | | | PDQSVKLLIYYTSSLHSGVPSRFSGSGSGTDYTLTINSLE |
| | | | AEDAATYYCQQYSKLPRTFGGGTKVEIK |
| SEQ ID NO: 3038 | VL | C-H-VL.39 | EIVMTQTPLSLSITPGEQASISCSASQGISNYLNWYLQKA |
| | | | RPVVKLLIYYTSSLHSGVPDRFSGSGSGTDYTLKISRVEA |
| | | | EDFGVYYCQQYSKLPRTFGGGTKVEIK |
| SEQ ID NO: 3039 | VL | C-H-VL.40 | EIVMTQTPLSLSITPGEQASMSCSASQGISNYLNWYLQK |
| | | | ARPVVKLLIYYTSSLHSGVPDRFSGSGSGTDYTLKISRVE |
| | | | AEDFGVYYCQQYSKLPRTFGGGTKVEIK |

Humanized antibody C Variable HEAVY chain (VH)

| | | | |
|---|---|---|---|
| SEQ ID NO: 3040 | VH | C-H-VH.1 | QVTLKESGPVLVKPTETLTLTCTVSGFSLTAYGVNWVR |
| | | | QPPGKALEWLGMIWGDGNTDYNSALKSRLTISKDNSKS |
| | | | QVVLTMTNMDPVDTATYYCARDRVTATLYAMDYWG |
| | | | QGTLVTVSS |
| SEQ ID NO: 3041 | VH | C-H-VH.2 | QVTLKESGPALVKPTETLTLTCTVSGFSLTAYGVNWVR |
| | | | QPPGKALEWLGMIWGDGNTDYNSALKSRLIISKDNSKS |
| | | | QVVLTMTNMDPVDTATYYCARDRVTATLYAMDYWG |
| | | | QGTLVTVSS |
| SEQ ID NO: 3042 | VH | C-H-VH.3 | QVTLKESGPALVKPTQTLTLTCTVSGFSLTAYGVNWVR |
| | | | QPPGKALEWLGMIWGDGNTDYNSALKSRLTISKDNSKS |
| | | | QVVLTMTNMDPVDTATYYCARDRVTATLYAMDYWG |
| | | | QGTLVTVSS |
| SEQ ID NO: 3043 | VH | C-H-VH.4 | QVQLQESGPGLVKPSGTLSLTCAVSGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3044 | VH | C-H-VH.5 | QVTLKESGPTLVKPTQTLTLTCTVSGFSLTAYGVNWVR |
| | | | QPPGKALEWLGMIWGDGNTDYNSALKSRLTITKDNSKS |
| | | | QVVLTMTNMDPVDTATYYCARDRVTATLYAMDYWG |
| | | | QGTLVTVSS |
| SEQ ID NO: 3045 | VH | C-H-VH.6 | QVTLKESGPALVKPTQTLTLTCTVSGFSLTAYGVNWVR |
| | | | QPPGKALEWLGMIWGDGNTDYNSALKSRLTITKDNSKS |
| | | | QVVLTMTNMDPVDTATYYCARDRVTATLYAMDYWG |
| | | | QGTLVTVSS |
| SEQ ID NO: 3046 | VH | C-H-VH.7 | QVQLQESGPGLVKPSQTLSLTCTVSGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3047 | VH | C-H-VH.8 | QVQLQESGPGLVKPSETLSLTCTVSGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3048 | VH | C-H-VH.9 | QVQLQESGPGLVKPSQTLSLTCAVSGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3049 | VH | C-H-VH.10 | QVQLQESGPGLVKPSDTLSLTCTVSGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3050 | VH | C-H-VH.11 | QVQLQESGPGLVKPSQTLSLTCTVSGFSLTAYGVNWVR |
| | | | QHPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3051 | VH | C-H-VH.12 | QVQLQESGPGLVKPSQTLSLTCTVSGFSLTAYGVNWVR |
| | | | QPAGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3052 | VH | C-H-VH.13 | QVQLQESGPGLVKPSQTLSLTCAVSGFSLTAYGVNWVR |
| | | | QPPGKGLEWLGMIWGDGNTDYNSALKSRLTISKDNSKS |
| | | | QVSLKLSSVTAVDTAVYYCARDRVTATLYAMDYWGQ |
| | | | GTLVTVSS |
| SEQ ID NO: 3053 | VH | C-H-VH.14 | QVQLQESGPGLVKPSETLSLTCTVSGFSLTAYGVNWVR |
| | | | QPPGKGLEWLGMIWGDGNTDYNSALKSRLTISKDNSKS |
| | | | HVSLKLSSVTAADTAVYYCARDRVTATLYAMDYWGQ |
| | | | GTLVTVSS |
| SEQ ID NO: 3054 | VH | C-H-VH.15 | QVQLQESGPGLVKPSETLSLTCAVSGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3055 | VH | C-H-VH.16 | QVQLQESGPGLVKPSQTLSLTCAVYGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3056 | VH | C-H-VH.17 | RVQLQESGPGLVKPSETLSLTCTVSGFSLTAYGVNWVR |
| | | | QPPGKGLEWLGMIWGDGNTDYNSALKSRLTISKDNSKS |
| | | | QVPLKLSSVTAADTAVYYCARDRVTATLYAMDYWGQ |
| | | | GTLVTVSS |
| SEQ ID NO: 3057 | VH | C-H-VH.18 | QVQLQESGPGLVKPSQTLSLTCTVSGFSLTAYGVNWVR |
| | | | QHPGKGLEWLGMIWGDGNTDYNSALKSLLTISKDNSKS |
| | | | QVSLKLSSVTAADTAVYYCARDRVTATLYAMDYWGQ |
| | | | GTLVTVSS |
| SEQ ID NO: 3058 | VH | C-H-VH.19 | QVQLQESGPGLVKPSDTLSLTCAVSGFSLTAYGVNWVR |
| | | | QPPGKGLEWLGMIWGDGNTDYNSALKSRLTISKDNSKS |
| | | | QVSLKLSSVTALDTAVYYCARDRVTATLYAMDYWGQ |
| | | | GTLVTVSS |
| SEQ ID NO: 3059 | VH | C-H-VH.20 | QVQLQESGPGLVKPSDTLSLTCAVSGFSLTAYGVNWVR |
| | | | QPPGKGLEWLGMIWGDGNTDYNSALKSRLTISKDNSKS |
| | | | QVSLKLSSVTAVDTAVYYCARDRVTATLYAMDYWGQ |
| | | | GTLVTVSS |
| SEQ ID NO: 3060 | VH | C-H-VH.21 | QVQLQESGSGLVKPSQTLSLTCAVSGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3061 | VH | C-H-VH.22 | EVQLVESGGGLVQPGRSLRLSCTVSGFSLTAYGVNWVR |
| | | | QAPGKGLEWLGMIWGDGNTDYNSALKSRLTISKDNSKS |
| | | | IVYLQMNSLKTEDTAVYYCARDRVTATLYAMDYWGQ |
| | | | GTLVTVSS |
| SEQ ID NO: 3062 | VH | C-H-VH.23 | EVQLVESGGGLVQPGPSLRLSCTVSGFSLTAYGVNWVR |
| | | | QAPGKGLEWLGMIWGDGNTDYNSALKSRLTISKDNSKS |
| | | | IVYLQMNSLKTEDTAVYYCARDRVTATLYAMDYWGQ |
| | | | GTLVTVSS |
| SEQ ID NO: 3063 | VH | C-H-VH.24 | QVQLQESGSGLVKPSQTLSLTCAVSGFSLTAYGVNWVR |
| | | | QSPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3064 | VH | C-H-VH.25 | QVQLQESGPGLVKPSETLSLTCTVSGFSLTAYGVNWVR |
| | | | QPAGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3065 | VH | C-H-VH.26 | EVQLVESGGGLVKPGRSLRLSCTVSGFSLTAYGVNWVR |
| | | | QAPGKGLEWLGMIWGDGNTDYNSALKSRLTISKDNSKS |
| | | | IVYLQMNSLKTEDTAVYYCARDRVTATLYAMDYWGQ |
| | | | GTLVTVSS |
| SEQ ID NO: 3066 | VH | C-H-VH.27 | QVQLQESGPGLVKPSETLSLTCAVYGFSLTAYGVNWVR |
| | | | QPPGKGLEWLGMIWGDGNTDYNSALKSRLTISKDNSKS |
| | | | QVYLKLSSVTAADTAVYYCARDRVTATLYAMDYWGQ |
| | | | GTLVTVSS |
| SEQ ID NO: 3067 | VH | C-H-VH.28 | QVQLQESGPGLVKPSDTLSLTCAVSGFSLTAYGVNWVR |
| | | | QPPGKGLEWLGMIWGDGNTDYNSALKSRLTISKDNSKS |
| | | | QVSLKLSSVTAVDTGVYYCARDRVTATLYAMDYWGQ |
| | | | GTLVTVSS |
| SEQ ID NO: 3068 | VH | C-H-VH.29 | EVQLVESGGGLVQPGGSLRLSCAVSGFSLTAYGVNWV |
| | | | RQAPGKGLEWLGMIWGDGNTDYNSALKSRLTISKDNS |
| | | | KSSVYLQMNSLKTEDTAVYYCARDRVTATLYAMDYW |
| | | | GQGTLVTVSS |
| SEQ ID NO: 3069 | VH | C-H-VH.30 | EVQLVESGGGLVKPGGSLRLSCAVSGFSLTAYGVNWV |
| | | | RQAPGKGLEWLGMIWGDGNTDYNSALKSRLTISKDNS |
| | | | KSTVYLQMNSLKTEDTAVYYCARDRVTATLYAMDYW |
| | | | GQGTLVTVSS |
| SEQ ID NO: 3070 | VH | C-H-VH.31 | QVQLQQSGPGLVKPSQTLSLTCAVSGFSLTAYGVNWVR |
| | | | QSPSRGLEWLGMIWGDGNTDYNSALKSRLTINKDNSKS |
| | | | QVSLQLNSVTPEDTAVYYCARDRVTATLYAMDYWGQ |
| | | | GTLVTVSS |
| SEQ ID NO: 3071 | VH | C-H-VH.32 | QVQLVESGGGLVQPGGSLRLSCSVSGFSLTAYGVNWVR |
| | | | QAPGKGLEWLGMIWGDGNTDYNSALKSRLTISKDNSKS |
| | | | TVYLQMNSLRAEDTAVYYCARDRVTATLYAMDYWGQ |
| | | | GTLVTVSS |
| SEQ ID NO: 3072 | VH | C-H-VH.33 | QVQLQQWGAGLLKPSETLSLTCAVYGFSLTAYGVNWV |
| | | | RQPPGKGLEWLGMIWGDGNTDYNSALKSRLTISKDNSK |
| | | | SQVSLKLSSVTAADTAVYYCARDRVTATLYAMDYWG |
| | | | QGTLVTVSS |
| SEQ ID NO: 3073 | VH | C-H-VH.34 | QVQLVESGGGVVQPGRSLRLSCAVSGFSLTAYGVNWV |
| | | | RQAPGKGLEWLGMIWGDGNTDYNSALKSRLTISKDNST |
| | | | STVFLQMNSLRAEDTAVYYCARDRVTATLYAMDYWG |
| | | | QGTLVTVSS |
| SEQ ID NO: 3074 | VH | C-H-VH.35 | EVQLVESGGGLVQPGGSLRLSCAVSGFSLTAYGVNWV |
| | | | RQAPGKGLEWLGMIWGDGNTDYNSALKSRLTISKDNS |
| | | | KSTVYLQMNSLRAEDTAVYYCARDRVTATLYAMDYW |
| | | | GQGTLVTVSS |
| SEQ ID NO: 3075 | VH | C-H-VH.36 | EVQLVESGGGLVQPGGSLRLSCAVSGFSLTAYGVNWV |
| | | | RQAPGKGLEWLGMIWGDGNTDYNSALKSRLTISKDNA |
| | | | KSSVYLQMNSLRDEDTAVYYCARDRVTATLYAMDYW |
| | | | GQGTLVTVSS |
| SEQ ID NO: 3076 | VH | C-H-VH.37 | EVQLLESGGGLVQPGGSLRLSCAVSGFSLTAYGVNWVR |
| | | | QAPGKGLEWLGMIWGDGNTDYNSALKSRLTISKDNSKS |
| | | | TVYLQMNSLRAEDTAVYYCARDRVTATLYAMDYWGQ |
| | | | GTLVTVSS |
| SEQ ID NO: 3077 | VH | C-H-VH.38 | QVQLVESGGGLVKPGGSLRLSCAVSGFSLTAYGVNWV |
| | | | RQAPGKGLEWLGMIWGDGNTDYNSALKSRLTISKDNA |
| | | | KSSVYLQMNSLRAEDTAVYYCARDRVTATLYAMDYW |
| | | | GQGTLVTVSS |
| SEQ ID NO: 3078 | VH | C-H-VH.39 | EVQLVESGGGLVQPGGSLKLSCAVSGFSLTAYGVNWV |
| | | | RQASGKGLEWLGMIWGDGNTDYNSALKSRLTISKDNS |
| | | | KSTVYLQMNSLKTEDTAVYYCARDRVTATLYAMDYW |
| | | | GQGTLVTVSS |
| SEQ ID NO: 3079 | VH | C-H-VH.40 | QVQLLESGGGLVKPGGSLRLSCAVSGFSLTAYGVNWV |
| | | | RQAPGKGLEWLGMIWGDGNTDYNSALKSRLTISKDNA |
| | | | KSSVYLQMNSLRAEDTAVYYCARDRVTATLYAMDYW |
| | | | GQGTLVTVSS |
| SEQ ID NO: 3080 | VH | C-H-VH.41 | QVQLVESGGGVVQPGRSLRLSCAVSGFSLTAYGVNWV |
| | | | RQAPGKGLEWLGMIWGDGNTDYNSALKSRLTISKDNS |
| | | | KSTVYLQMNSLRAEDTAVYYCARDRVTATLYAMDYW |
| | | | GQGTLVTVSS |
| SEQ ID NO: 3081 | VH | C-H-VH.42 | QVQLVESGGGVVQPGRSLRLSCAVSGFSLTAYGVNWV |
| | | | RQAPGKGLEWLGMIWGDGNTDYNSALKSRLTISKDNS |
| | | | KSRVYLQMNSLRAEDTAVYYCARDRVTATLYAMDYW |
| | | | GQGTLVTVSS |
| SEQ ID NO: 3082 | VH | C-H-VH.43 | QVQLVESGGGVVQPGRSLRLSCAVSGFSLTAYGVNWV |
| | | | RQAPGKGLEWLGMIWGDGNTDYNSALKSRLAISKDNS |
| | | | KSTVYLQMNSLRAEDTAVYYCARDRVTATLYAMDYW |
| | | | GQGTLVTVSS |
| SEQ ID NO: 3083 | VH | C-H-VH.44 | QVQLVESGGGVVQPGGSLRLSCAVSGFSLTAYGVNWV |
| | | | RQAPGKGLEWLGMIWGDGNTDYNSALKSRLTISKDNS |
| | | | KSTVYLQMNSLRAEDTAVYYCARDRVTATLYAMDYW |
| | | | GQGTLVTVSS |
| SEQ ID NO: 3084 | VH | C-H-VH.45 | EVQLVESGGGLVQPGGSLRLSCAVSGFSLTAYGVNWV |
| | | | RQAPGKGLEWLGMIWGDGNTDYNSALKSRLTISKDNA |
| | | | KSTVYLQMNSLRAEDTAVYYCARDRVTATLYAMDYW |
| | | | GQGTLVTVSS |
| SEQ ID NO: 3085 | VH | C-H-VH.46 | EVQLVESGGGLVQPGGSLRLSCAVSGFSLTAYGVNWV |
| | | | RQAPGKGLEWLGMIWGDGNTDYNSALKSRLTISKDNA |
| | | | KSSVYLQMNSLRAEDTAVYYCARDRVTATLYAMDYW |
| | | | GQGTLVTVSS |
| SEQ ID NO: 3086 | VH | C-H-VH.47 | EVQLVESGGVVVQPGGSLRLSCAVSGFSLTAYGVNWV |
| | | | RQAPGKGLEWLGMIWGDGNTDYNSALKSRLTISKDNS |
| | | | KSSVYLQMNSLRTEDTALYYCARDRVTATLYAMDYW |
| | | | GQGTLVTVSS |
| SEQ ID NO: 3087 | VH | C-H-VH.48 | EVQLVESGGGLVQPGGSLRLSCAVSGFSLTAYGVNWV |
| | | | RQAPGKGLEWLGMIWGDGNTDYNSALKSRLTISKHNS |
| | | | KSTVYLQMNSLRAEDTAVYYCARDRVTATLYAMDYW |
| | | | GQGTLVTVSS |
| SEQ ID NO: 3088 | VH | C-H-VH.49 | EVQLVESGGGLVKPGGSLRLSCAVSGFSLTAYGVNWV |
| | | | RQAPGKGLEWLGMIWGDGNTDYNSALKSRLTISKDNA |
| | | | KSSVYLQMNSLRAEDTAVYYCARDRVTATLYAMDYW |
| | | | GQGTLVTVSS |
| SEQ ID NO: 3089 | VH | C-H-VH.50 | EVQLVESGGGLIQPGGSLRLSCAVSGFSLTAYGVNWVR |
| | | | QPPGKGLEWLGMIWGDGNTDYNSALKSRLTISKDNSKS |
| | | | TVYLQMNSLRAEDTAVYYCARDRVTATLYAMDYWGQ |
| | | | GTLVTVSS |

TABLE 10C
Exemplary agents that comprise a first binder that binds to a first
target molecule, wherein the exemplary first binder comprises anti-TCRβ
V5 antibodies. Exemplary first target molecule comprises TCRVB 5
(e.g., TCRVB 5-1). Exemplary anti-TCRβ V13 antibodies include, but are
not limited to, BLM0272, which binds to human TRBV5-1. CDRs are underlined.
BLM0272, also referred to as BJ1307or BJ1309
Binds to human TCRVβ 5-1

| | | |
|---|---|---|
| SEQ ID NO: 3338 | Heavy chain (VH- | QVQLVQSGAEVKKPGASVKVSCKASGYTFTDYNIHWVRQAP |
| | CH) | GQGLEWMGYINPYNGRTGYNQKFKARVTMTVDKSTSTAYM |
| | | ELSSLRSEDTAVYYCARWDGSSYFDYWGQGTLVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHKP |
| | | SNTKVDKRVEPKSCDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVCTLPPSREEMTKNQVSLSCAVKGFYP |
| | | SDIAVEWESNGQPENNYKTTPPVLDSDGSFFLVSKLTVDKSR |
| | | WQQGNVFSCSVMHEALHNHYTQKSLSLSPGK |
| SEQ ID NO: 3339 | Light chain (VL-CL) | EIVLTQSPATLSVSPGERATLSCSASSSVSYMHWYQQKPGQA |
| | | PRPLIYEISKLASGIPARFSGSGSGTEYTLTISSLQSEDFAVYYC |
| | | QQWNYPLLTFGQGTKLEIKRTVAAPSVFIFPPSDEQLKSGTAS |
| | | VVCLLNNFYPREAKVQWKVDNALQSGNSQESVTEQDSKDST |
| | | YSLSSTLTLSKADYEKHKVYACEVTHQGLSSPVTKSFNRGEC |
| SEQ ID NO: 3340 | IL-2 C125A - Heavy | APTSSSTKKTQLQLEHLLLDLQMILNGINNYKNPKLTRMLTF |
| | Chain Constant | KFYMPKKATELKHLQCLEEELKPLEEVLNLAQSKNFHLRPRD |
| | Domain (Fc Region) | LISNINVIVLELKGSETTFMCEYADETATIVEFLNRWITFAQSII |
| | | STLTGGGGSGGGGSDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVYTLPPCREEMTKNQVSLWCLVKGFY |
| | | PSDIAVEWESNGQPENNYKTTPPVLDSDGSFFLYSKLTVDKS |
| | | RWQQGNVFSCSVMHEALHNHYTQKSLSLSPGK |
| SEQ ID NO: 3341 | HC CDR1 | GYTFTDYNIH |
| SEQ ID NO: 3342 | HC CDR2 | YINPYNGRTGYNQKFKA |
| SEQ ID NO: 3343 | HC CDR3 | WDGSSYFDY |
| SEQ ID NO: 3344 | LC CDR1 | SASSSVSYMH |
| SEQ ID NO: 3345 | LC CDR2 | EISKLAS |
| SEQ ID NO: 3346 | LC CDR3 | QQWNYPLLT |
| SEQ ID NO: 3445 | VH | QVQLVQSGAEVKKPGASVKVSCKASGYTFTDYNIHWVRQAP |
| | | GQGLEWMGYINPYNGRTGYNQKFKARVTMTVDKSTSTAYM |
| | | ELSSLRSEDTAVYYCARWDGSSYFDYWGQGTLVTVSS |
| SEQ ID NO: 3446 | VL | EIVLTQSPATLSVSPGERATLSCSASSSVSYMHWYQQKPGQA |
| | | PRPLIYEISKLASGIPARFSGSGSGTEYTLTISSLQSEDFAV |
| | | YYCQQWNYPLLTFGQGTKLEIK |
| SEQ ID NO: 3649 | Heavy chain constant | ASTKGPSVFPLAPSSKSTSGGTAALGCLVKDYFPEPVTVSWN |
| | region (CH1-CH2- | SGALTSGVHTFPAVLQSSGLYSLSSVVTVPSSSLGTQTYICNV |
| | CH3) Hole | NHKPSNTKVDKRVEPKSCDKTHTCPPCPAPELLGGPSVFLFPP |
| | | KPKDTLMISRTPEVTCVVVDVSHEDPEVKFNWYVDGVEVHN |
| | | AKTKPREEQYASTYRVVSVLTVLHQDWLNGKEYKCKVSNKA |
| | | LPAPIEKTISKAKGQPREPQVCTLPPSREEMTKNQVSLSCAV |
| | | KGFYPSDIAVEWESNGQPENNYKTTPPVLDSDGSFFLVSKLT |
| | | VDKSRWQQGNVFSCSVMHEALHNHYTQKSLSLSPGK |
| SEQ ID NO: 3447 | Heavy chain hinge - | DKTHTCPPCPAPELLGGPSVFLFPPKPKDTLMISRTPEVTCVV |
| | Fc region (CH2-CH3) | VDVSHEDPEVKFNWYVDGVEVHNAKTKPREEQYASTYRVV |
| | Hole | SVLTVLHQDWLNGKEYKCKVSNKALPAPIEKTISKAKGQPRE |
| | | PQVCTLPPSREEMTKNQVSLSCAVKGFYPSDIAVEWESNGQP |
| | | ENNYKTTPPVLDSDGSFFLVSKLTVDKSRWQQGNVFSCSVM |
| | | HEALHNHYTQKSLSLSPGK |
| SEQ ID NO: 3644 | Light chain constant | RTVAAPSVFIFPPSDEQLKSGTASVVCLLNNFYPREAKVQWK |
| | region | VDNALQSGNSQESVTEQDSKDSTYSLSSTLTLSKADYEKHKV |
| | | YACEVTHQGLSSPVTKSFNRGEC |
| SEQ ID NO: 2270 | IL-2 C125A | APTSSSTKKTQLQLEHLLLDLQMILNGINNYKNPKLTRMLTF |
| | | KFYMPKKATELKHLQCLEEELKPLEEVLNLAQSKNFHLRPRD |
| | | LISNINVIVLELKGSETTFMCEYADETATIVEFLNRWITFAQ |
| | | SIISTLT |
| SEQ ID NO: 3308 | Linker | GGGGSGGGGS |
| SEQ ID NO: 3648 | Heavy chain hinge - | DKTHTCPPCPAPELLGGPSVFLFPPKPKDTLMISRTPEVTCV |
| | Fc region (CH2-CH3) | VVDVSHEDPEVKFNWYVDGVEVHNAKTKPREEQYASTYRVV |
| | knob | SVLTVLHQDWLNGKEYKCKVSNKALPAPIEKTISKAKGQPRE |
| | | PQVYTLPPCREEMTKNQVSLWCLVKGFYPSDIAVEWESNGQ |
| | | PENNYKTTPPVLDSDGSFFLYSKLTVDKSRWQQGNVFSCSV |
| | | MHEALHNHYTQKSLSLSPGK |

TABLE 11
Amino acid sequences for anti TCRβ V5 antibodies. Amino acid and
nucleotide sequences for murine and humanized antibody molecules
which bind to TCRVB 5 (e.g., TCRVB 5-5 or TCRVB 5-6). The amino
acid of the heavy and light chain CDRs, and the amino acid and
nucleotide sequences of the heavy and light chain variable
regions, and the heavy and light chains are shown.
Murine antibody E, also referred to as MH3-2

| | | |
|---|---|---|
| SEQ ID NO: 1298 | HC CDR1 (Kabat) | SSWMN |
| SEQ ID NO: 1299 | HC CDR2 (Kabat) | RIYPGDGDTKYNGKFKG |
| SEQ ID NO: 1300 | HC CDR3 (Kabat) | RGTGGWYFDV |
| SEQ ID NO: 1302 | HC CDR1 (Chothia) | GYAFSSS |
| SEQ ID NO: 1303 | HC CDR2 (Chothia) | YPGDGD |
| SEQ ID NO: 1301 | HC CDR3 (Chothia) | RGTGGWYFDV |
| SEQ ID NO: 1304 | HC CDR1 (Combined) | GYAFSSSWMN |
| SEQ ID NO: 1299 | HC CDR2 (Combined)) | RIYPGDGDTKYNGKFKG |
| SEQ ID NO: 1301 | HC CDR3(Combined) | RGTGGWYFDV |
| SEQ ID NO: 1305 | LC CDR1 (Kabat) | RASESVDSSGNSFMH |
| SEQ ID NO: 1306 | LC CDR2 (Kabat) | RASNLES |
| SEQ ID NO: 1307 | LC CDR3 (Kabat) | QQSFDDPFT |
| SEQ ID NO: 1308 | LC CDR1 (Chothia) | SESVDSSGNSF |
| SEQ ID NO: 1306 | LC CDR2 (Chothia) | RASNLES |
| SEQ ID NO: 1307 | LC CDR3 (Chothia) | QQSFDDPFT |
| SEQ ID NO: 1305 | LC CDR1 (Combined) | RASESVDSSGNSFMH |
| SEQ ID NO: 1306 | LC CDR2 (Combined) | RASNLES |
| SEQ ID NO: 1307 | LC CDR3(Combined) | QQSFDDPFT |
| SEQ ID NO: 3091 | VH | QVQLQQSGPELVKPGASVKISCKASGYAFSSSWMNWV |
| | | KQRPGQGLEWIGRIYPGDGDTKYNGKFKGKATLTADKS |
| | | SSTAYMHLSSLTSVDSAVYFCARRGTGGWYFDVWGAG |
| | | TTVTVSS |
| SEQ ID NO: 3284 | Heavy chain | METDTLLLWVLLLWVPGSTGQVQLQQSGPELVKPGAS |
| | | VKISCKASGYAFSSSWMNWVKQRPGQGLEWIGRIYPGD |
| | | GDTKYNGKFKGKATLTADKSSSTAYMHLSSLTSVDSAV |
| | | YFCARRGTGGWYFDVWGAGTTVTVSSAKTTAPSVYPL |
| | | APVCGDTTGSSVTLGCLVKGYFPEPVTLTWNSGSLSSG |
| | | VHTFPAVLQSDLYTLSSSVTVTSSTWPSQSITCNVAHPA |
| | | SSTKVDKKIEPRGPTIKPCPPCKCPAPNLLGGPSVFIFPPK |
| | | IKDVLMISLSPIVTCVVVDVSEDDPDVQISWFVNNVEVH |
| | | TAQTQTHREDYNSTLRVVSALPIQHQDWMSGKEFKCK |
| | | VNNKDLPAPIERTISKPKGSVRAPQVYVLPPPEEEMTKK |
| | | QVTLTCMVTDFMPEDIYVEWTNNGKTELNYKNTEPVL |
| | | DSDGSYFMYSKLRVEKKNWVERNSYSCSVVHEGLHNH |
| | | HTTKSFSRTPGK |
| SEQ ID NO: 3092 | VL | DIVLTQSPASLAVSLGQRATISCRASESVDSSGNSFMHW |
| | | YQQKPGQPPQLLIYRASNLESGIPARFSGSGSRTDFTLTI |
| | | NPVEADDVATFYCQQSFDDPFTFGSGTKLEIK |
| SEQ ID NO: 3285 | Light chain | METDTLLLWVLLLWVPGSTGDIVLTQSPASLAVSLGQR |
| | | ATISCRASESVDSSGNSFMHWYQQKPGQPPQLLIYRASN |
| | | LESGIPARFSGSGSRTDFTLTINPVEADDVATFYCQQSFD |
| | | DPFTFGSGTKLEIKRADAAPTVSIFPPSSEQLTSGGASVV |
| | | CFLNNFYPKDINVKWKIDGSERQNGVLNSWTDQDSKDS |
| | | TYSMSSTLTLTKDEYERHNSYTCEATHKTSTSPIVKSFN |
| | | RNEC |

Humanized antibody E (E-H antibody)
Variable light chain (VL)

| | | | |
|---|---|---|---|
| SEQ ID NO: 3093 | VL | E-H.1 | DIVLTQSPDSLAVSLGERATINCRASESVDSSGNSFMHWYQQ |
| | | | KPGQPPQLLIYRASNLESGVPDRFSGSGSRTDFTLTISSLQAED |
| | | | VAVYYCQQSFDDPFTFGQGTKLEIK |
| SEQ ID NO: 3094 | VL | E-H.2 | EIVLTQSPATLSLSPGERATLSCRASESVDSSGNSFMHWYQQ |
| | | | KPGQAPQLLIYRASNLESGIPARFSGSGSRTDFTLTISSLEPEDF |
| | | | AVYYCQQSFDDPFTFGQGTKLEIK |
| SEQ ID NO: 3095 | VL | E-H.3 | EIVLTQSPATLSLSPGERATLSCRASESVDSSGNSFMHWYQQ |
| | | | KPGQAPQLLIYRASNLESGIPARFSGSGSRTDFTLTISRLEPED |
| | | | FAVYYCQQSFDDPFTFGQGTKLEIK |
| SEQ ID NO: 3096 | VL | E-H.4 | EIVLTQSPATLSLSPGERATLSCRASESVDSSGNSFMHWYQQ |
| | | | KPGQAPQLLIYRASNLESGIPARFSGSGSRTDFTLTISSLQPED |
| | | | FAVYYCQQSFDDPFTFGQGTKLEIK |
| SEQ ID NO: 3097 | VL | E-H.5 | DIQLTQSPSSLSASVGDRVTITCRASESVDSSGNSFMHWYQQ |
| | | | KPGQAPQLLIYRASNLESGVPSRFSGSGSRTDFTLTISSLQPED |
| | | | VATYYCQQSFDDPFTFGQGTKLEIK |
| SEQ ID NO: 3098 | VL | E-H.6 | EIVLTQSPATLSLSPGERATLSCRASESVDSSGNSFMHWYQQ |
| | | | KPGQAPQLLIYRASNLESGIPARFSGSGPRTDFTLTISSLEPEDF |
| | | | AVYYCQQSFDDPFTFGQGTKLEIK |
| SEQ ID NO: 3099 | VL | E-H.7 | EIVLTQSPATLSLSPGERATLSCRASESVDSSGNSFMHWYQQ |
| | | | KPGQAPQLLIYRASNLESGIPDRFSGSGSRTDFTLTISRLEPED |
| | | | FAVYYCQQSFDDPFTFGQGTKLEIK |
| SEQ ID NO: 3100 | VL | E-H.8 | DIQLTQSPSSLSASVGDRVTITCRASESVDSSGNSFMHWYQQ |
| | | | KPGKVPQLLIYRASNLESGVPSRFSGSGSRTDFTLTISSLQPED |
| | | | VATYYCQQSFDDPFTFGQGTKLEIK |
| SEQ ID NO: 3101 | VL | E-H.9 | DIQLTQSPSSLSASVGDRVTITCRASESVDSSGNSFMHWYQQ |
| | | | KPGKTPQLLIYRASNLESGIPSRFSGSGSRTDFTLTIRSLQPEDF |
| | | | ATYYCQQSEDDPFTFGQGTKLEIK |
| SEQ ID NO: 3102 | VL | E-H.10 | EIVLTQSPGTLSLSPGERATLSCRASESVDSSGNSFMHWYQQ |
| | | | KPGQAPQLLIYRASNLESGIPDRFSGSGSRTDFTLTISRLEPED |
| | | | FAVYYCQQSFDDPFTFGQGTKLEIK |
| SEQ ID NO: 3103 | VL | E-H.11 | EIVLTQSPATLSLSPGERATLSCRASESVDSSGNSFMHWYQQ |
| | | | KPGLAPQLLIYRASNLESGIPDRFSGSGSRTDFTLTISRLEPEDF |
| | | | AVYYCQQSFDDPFTFGQGTKLEIK |
| SEQ ID NO: 3104 | VL | E-H.12 | DIQLTQSPSSLSASVGDRVTITCRASESVDSSGNSFMHWYQQ |
| | | | KPGKAPQLLIYRASNLESGVPSRFSGSGSRTDFTLTISSLQPED |
| | | | FATYYCQQSFDDPFTFGQGTKLEIK |
| SEQ ID NO: 3105 | VL | E-H.13 | DIQLTQSPSSVSASVGDRVTITCRASESVDSSGNSFMHWYQQ |
| | | | KPGKAPQLLIYRASNLESGVPSRFSGSGSRTDFTLTISSLQPED |
| | | | FATYYCQQSFDDPFTFGQGTKLEIK |
| SEQ ID NO: 3106 | VL | E-H.14 | AIQLTQSPSSLSASVGDRVTITCRASESVDSSGNSFMHWYQQ |
| | | | KPGKAPQLLIYRASNLESGVPSRFSGSGSRTDFTLTISSLQPED |
| | | | FATYYCQQSFDDPFTFGQGTKLEIK |
| SEQ ID NO: 3107 | VL | E-H.15 | DIQLTQSPSFLSASVGDRVTITCRASESVDSSGNSFMHWYQQ |
| | | | KPGKAPQLLIYRASNLESGVPSRFSGSGSRTEFTLTISSLQPED |
| | | | FATYYCQQSFDDPFTFGQGTKLEIK |
| SEQ ID NO: 3108 | VL | E-H.16 | DIQLTQSPSSLSASVGDRVTITCRASESVDSSGNSFMHWYQQ |
| | | | KPGKAPQLLIYRASNLESGVPSRFSGSGSRTDFTFTISSLQPEDI |
| | | | ATYYCQQSFDDPFTFGQGTKLEIK |
| SEQ ID NO: 3109 | VL | E-H.17 | EIVLTQSPATLSVSPGERATLSCRASESVDSSGNSFMHWYQQ |
| | | | KPGQAPQLLIYRASNLESGIPARFSGSGSRTEFTLTISILQSEDF |
| | | | AVYYCQQSFDDPFTFGQGTKLEIK |
| SEQ ID NO: 3110 | VL | E-H. 18 | EIVLTQSPATLSVSPGERATLSCRASESVDSSGNSFMHWYQQ |
| | | | KPGQAPQLLIYRASNLESGIPARFSGSGSRTEFTLTISSLQSEDF |
| | | | AVYYCQQSFDDPFTFGQGTKLEIK |
| SEQ ID NO: 3111 | VL | E-H.19 | AIRLTQSPFSLSASVGDRVTITCRASESVDSSGNSFMHWYQQ |
| | | | KPAKAPQLFIYRASNLESGVPSRFSGSGSRTDFTLTISSLQPED |
| | | | FATYYCQQSFDDPFTFGQGTKLEIK |
| SEQ ID NO: 3112 | VL | E-H.20 | DIQLTQSPSSLSASVGDRVTITCRASESVDSSGNSFMHWYQQ |
| | | | KPGKAPQSLIYRASNLESGVPSRFSGSGSRTDFTLTISSLQPED |
| | | | FATYYCQQSFDDPFTFGQGTKLEIK |
| SEQ ID NO: 3113 | VL | E-H.21 | DIQLTQSPSSLSASVGDRVTITCRASESVDSSGNSFMHWYQQ |
| | | | KPGKAPQRLIYRASNLESGVPSRFSGSGSRTEFTLTISNLQPED |
| | | | FATYYCQQSFDDPFTFGQGTKLEIK |
| SEQ ID NO: 3114 | VL | E-H.22 | DIQLTQSPSTLSASVGDRVTITCRASESVDSSGNSFMHWYQQ |
| | | | KPGKAPQLLIYRASNLESGVPSRFSGSGSRTEFTLTISSLQPDD |
| | | | FATYYCQQSFDDPFTFGQGTKLEIK |
| SEQ ID NO: 3115 | VL | E-H.23 | EIVLTQSPDFQSVTPKEKVTITCRASESVDSSGNSFMHWYQQ |
| | | | KPDQSPQLLIYRASNLESGVPSRFSGSGSRTDFTLTINSLEAED |
| | | | AATYYCQQSFDDPFTFGQGTKLEIK |
| SEQ ID NO: 3116 | VL | E-H.24 | DIQLTQSPSSLSASVGDRVTITCRASESVDSSGNSFMHWYQQ |
| | | | KPGKAPQSLIYRASNLESGVPSKFSGSGSRTDFTLTISSLQPED |
| | | | FATYYCQQSFDDPFTFGQGTKLEIK |
| SEQ ID NO: 3117 | VL | E-H.25 | DIQLTQSPSSLSASVGDRVTITCRASESVDSSGNSFMHWYQQ |
| | | | KPGKAPQRLIYRASNLESGVPSRFSGSGSRTEFTLTISSLQPED |
| | | | FATYYCQQSFDDPFTFGQGTKLEIK |
| SEQ ID NO: 3118 | VL | E-H.26 | DIVLTQTPLSLSVTPGQPASISCRASESVDSSGNSFMHWYLQK |
| | | | PGQPPQLLIYRASNLESGVPDRFSGSGSRTDFTLKISRVEAED |
| | | | VGVYYCQQSFDDPFTFGQGTKLEIK |
| SEQ ID NO: 3119 | VL | E-H.27 | DIQLTQSPSSLSASVGDRVTITCRASESVDSSGNSFMHWYQQ |
| | | | KPEKAPQSLIYRASNLESGVPSRFSGSGSRTDFTLTISSLQPED |
| | | | FATYYCQQSFDDPFTFGQGTKLEIK |
| SEQ ID NO: 3120 | VL | E-H.28 | EIVLTQSPPTLSLSPGERVTLSCRASESVDSSGNSFMHWYQQK |
| | | | PGQAPQLLIYRASNLESGIPARFSGSGSRTDFTLTISSLQPEDF |
| | | | AVYYCQQSFDDPFTFGQGTKLEIK |
| SEQ ID NO: 3121 | VL | E-H.29 | DIQLTQSPSAMSASVGDRVTITCRASESVDSSGNSFMHWYQQ |
| | | | KPGKVPQRLIYRASNLESGVPSRFSGSGSRTEFTLTISSLQPED |
| | | | FATYYCQQSFDDPFTFGQGTKLEIK |
| SEQ ID NO: 3122 | VL | E-H.30 | DIVLTQSPLSLPVTPGEPASISCRASESVDSSGNSFMHWYLQK |
| | | | PGQSPQLLIYRASNLESGVPDRFSGSGSRTDFTLKISRVEAED |
| | | | VGVYYCQQSFDDPFTFGQGTKLEIK |
| SEQ ID NO: 3123 | VL | E-H.31 | DIVLTQTPLSLPVTPGEPASISCRASESVDSSGNSFMHWYLQK |
| | | | PGQSPQLLIYRASNLESGVPDRFSGSGSRTDFTLKISRVEAED |
| | | | VGVYYCQQSFDDPFTFGQGTKLEIK |
| SEQ ID NO: 3124 | VL | E-H.32 | DIVLTQTPLSLSVTPGQPASISCRASESVDSSGNSFMHWYLQK |
| | | | PGQSPQLLIYRASNLESGVPDRFSGSGSRTDFTLKISRVEAED |
| | | | VGVYYCQQSFDDPFTFGQGTKLEIK |
| SEQ ID NO: 3125 | VL | E-H.33 | EIVLTQSPPTLSLSPGERVTLSCRASESVDSSGNSFMHWYQQK |
| | | | PGQAPQLLIYRASNLESSIPARFSGSGSRTDFTLTISSLQPED |
| | | | FAVYYCQQSFDDPFTFGQGTKLEIK |
| SEQ ID NO: 3126 | VL | E-H.34 | DIVLTQSPLSLPVTLGQPASISCRASESVDSSGNSFMHWYQQR |
| | | | PGQSPQRLIYRASNLESGVPDRFSGSGSRTDFTLKISRVEAED |
| | | | VGVYYCQQSFDDPFTFGQGTKLEIK |
| SEQ ID NO: 3127 | VL | E-H.35 | DIVLTQTPLSSPVTLGQPASISCRASESVDSSGNSFMHWYQQR |
| | | | PGQPPQLLIYRASNLESGVPDRFSGSGARTDFTLKISRVEAED |
| | | | VGVYYCQQSFDDPFTFGQGTKLEIK |
| SEQ ID NO: 3128 | VL | E-H.36 | DIVLTQSPAFLSVTPGEKVTITCRASESVDSSGNSFMHWYQQ |
| | | | KPDQAPQLLIYRASNLESGVPSRFSGSGSRTDFTFTISSLEAED |
| | | | AATYYCQQSFDDPFTFGQGTKLEIK |
| SEQ ID NO: 3129 | VL | E-H.37 | DIQLIQSPSFLSASVGDRVSIICRASESVDSSGNSFMHWYLQKP |
| | | | GKSPQLFIYRASNLESGVSSRFSGRGSRTDFTLTIISLKPEDFA |
| | | | AYYCQQSFDDPFTFGQGTKLEIK |
| SEQ ID NO: 3130 | VL | E-H.38 | EIVLTQTPLSLSITPGEQASISCRASESVDSSGNSFMHWYLQK |
| | | | ARPVPQLLIYRASNLESGVPDRFSGSGSRTDFTLKISRVEAED |
| | | | FGVYYCQQSFDDPFTFGQGTKLEIK |
| SEQ ID NO: 3131 | VL | E-H.39 | EIVLTQTPLSLSITPGEQASMSCRASESVDSSGNSFMHWYLQK |
| | | | ARPVPQLLIYRASNLESGVPDRFSGSGSRTDFTLKISRVEAED |
| | | | FGVYYCQQSFDDPFTFGQGTKLEIK |
| SEQ ID NO: 3132 | VL | E-H.40 | EITLTQSPAFMSATPGDKVNISCRASESVDSSGNSFMHWYQQ |
| | | | KPGEAPQFIIYRASNLESGIPPRFSGSGYRTDFTLTINNIESEDA |
| | | | AYYYCQQSFDDPFTFGQGTKLEIK |

Variable HEAVY chain (VH)

| | | | |
|---|---|---|---|
| SEQ ID NO: 3133 | VH | E-H. | QVQLVQSGAEVKKPGASVKVSCKASGYAFSSSWMNWVRQA |
| | | | PGQGLEWIGRIYPGDGDTKYNGKFKGRATLTADKSTSTAYM |
| | | | ELSSLRSEDTAVYYCARRGTGGWYFDVWGQGTTVTVSS |
| SEQ ID NO: 3134 | VH | E-H.2 | QVQLVQSGAEVKKPGSSVKVSCKASGYAFSSSWMNWVRQA |
| | | | PGQGLEWIGRIYPGDGDTKYNGKFKGRATLTADKSTSTAYM |
| | | | ELSSLRSEDTAVYYCARRGTGGWYFDVWGQGTTVTVSS |
| SEQ ID NO: 3135 | VH | E-H.3 | QVQLVQSGAEVKKPGASVKVSCKASGYAFSSSWMNWVRQA |
| | | | PGKGLEWIGRIYPGDGDTKYNGKFKGRATLTADKSTSTAYM |
| | | | ELSSLRSEDTAVYYCARRGTGGWYFDVWGQGTTVTVSS |
| SEQ ID NO: 3136 | VH | E-H.4 | QVQLVQSGAEVKKPGASVKVSCKASGYAFSSSWMNWVRQA |
| | | | PGQELEWIGRIYPGDGDTKYNGKFKGRATLTADKSISTAYME |
| | | | LSSLRSEDTATYYCARRGTGGWYFDVWGQGTTVTVSS |
| SEQ ID NO: 3137 | VH | E-H.5 | EVQLVQSGAEVKKPGATVKISCKASGYAFSSSWMNWVQQA |
| | | | PGKGLEWIGRIYPGDGDTKYNGKFKGRATLTADKSTSTAYM |
| | | | ELSSLRSEDTAVYYCARRGTGGWYFDVWGQGTTVTVSS |
| SEQ ID NO: 3138 | VH | E-H.6 | QVQLVQSGAEVKKTGSSVKVSCKASGYAFSSSWMNWVRQA |
| | | | PGQALEWIGRIYPGDGDTKYNGKFKGRATLTADKSMSTAYM |
| | | | ELSSLRSEDTAMYYCARRGTGGWYFDVWGQGTTVTVSS |
| SEQ ID NO: 3139 | VH | E-H.7 | QVQLVQSGAEVKKPGASVKVSCKASGYAFSSSWMNWVRQA |
| | | | PGQRLEWIGRIYPGDGDTKYNGKFKGRATLTADKSASTAYM |
| | | | ELSSLRSEDMAVYYCARRGTGGWYFDVWGQGTTVTVSS |
| SEQ ID NO: 3140 | VH | E-H.8 | QVQLVQSGAEVKKPGASVKVSCKASGYAFSSSWMNWVRQA |
| | | | PGQGLEWIGRIYPGDGDTKYNGKFKGRATLTADKSTSTAYM |
| | | | ELRSLRSDDMAVYYCARRGTGGWYFDVWGQGTTVTVSS |
| SEQ ID NO: 3141 | VH | E-H.9 | QVQLVQSGAEVKKPGASVKVSCKASGYAFSSSWMNWVRQA |
| | | | PGQRLEWIGRIYPGDGDTKYNGKFKGRATLTADKSASTAYM |
| | | | ELSSLRSEDTAVYYCARRGTGGWYFDVWGQGTTVTVSS |
| SEQ ID NO: 3142 | VH | E-H.10 | QVQLVQSGAEVKKPGASVKVSCKASGYAFSSSWMNWVRQA |
| | | | PGQGLEWIGRIYPGDGDTKYNGKFKGRATLTADKSTSTAYM |
| | | | ELRSLRSDDTAVYYCARRGTGGWYFDVWGQGTTVTVSS |
| SEQ ID NO: 3143 | VH | E-H.11 | QVQLVQSGAEVKKPGASVKVSCKASGYAFSSSWMNWVRQA |
| | | | PGQGLEWIGRIYPGDGDTKYNGKFKGRATLTADKSISTAYM |
| | | | ELSRLRSDDTAVYYCARRGTGGWYFDVWGQGTTVTVSS |
| SEQ ID NO: 3144 | VH | E-H.12 | QVQLVQSGAEVKKPGASVKVSCKASGYAFSSSWMNWVRQA |
| | | | PGQGLEWIGRIYPGDGDTKYNGKFKGRATLTADKSISTAYM |
| | | | ELSRLRSDDTVVYYCARRGTGGWYFDVWGQGTTVTVSS |
| SEQ ID NO: 3145 | VH | E-H.13 | QVQLVQSGAEVKKPGASVKVSCKASGYAFSSSWMNWVRQA |
| | | | PGQGLEWIGRIYPGDGDTKYNGKFKGWATLTADKSISTAYM |
| | | | ELSRLRSDDTAVYYCARRGTGGWYFDVWGQGTTVTVSS |
| SEQ ID NO: 3146 | VH | E-H.14 | QVQLVQSGAEVKKPGASVKVSCKASGYAFSSSWMNWVRQA |
| | | | TGQGLEWIGRIYPGDGDTKYNGKFKGRATLTANKSISTAYM |
| | | | ELSSLRSEDTAVYYCARRGTGGWYFDVWGQGTTVTVSS |
| SEQ ID NO: 3147 | VH | E-H.15 | QVQLVQSGSELKKPGASVKVSCKASGYAFSSSWMNWVRQA |
| | | | PGQGLEWIGRIYPGDGDTKYNGKFKGRAVLSADKSVSTAYL |
| | | | QISSLKAEDTAVYYCARRGTGGWYFDVWGQGTTVTVSS |
| SEQ ID NO: 3148 | VH | E-H.16 | QVQLVQSGPEVKKPGTSVKVSCKASGYAFSSSWMNWVRQA |
| | | | RGQRLEWIGRIYPGDGDTKYNGKFKGRATLTADKSTSTAYM |
| | | | ELSSLRSEDTAVYYCARRGTGGWYFDVWGQGTTVTVSS |
| SEQ ID NO: 3149 | VH | E-H.17 | EVQLVQSGAEVKKPGESLKISCKASGYAFSSSWMNWVRQMP |
| | | | GKGLEWIGRIYPGDGDTKYNGKFKGQATLSADKSISTAYLQ |
| | | | WSSLKASDTAMYYCARRGTGGWYFDVWGQGTTVTVSS |
| SEQ ID NO: 3150 | VH | E-H.18 | QVQLVQSGSELKKPGASVKVSCKASGYAFSSSWMNWVRQA |
| | | | PGQGLEWIGRIYPGDGDTKYNGKFKGRAVLSADKSVSMAYL |
| | | | QISSLKAEDTAVYYCARRGTGGWYFDVWGQGTTVTVSS |
| SEQ ID NO: 3151 | VH | E-H.19 | QVQLVQSGHEVKQPGASVKVSCKASGYAFSSSWMNWVPQA |
| | | | PGQGLEWIGRIYPGDGDTKYNGKFKGRAVLSADKSASTAYL |
| | | | QISSLKAEDMAMYYCARRGTGGWYFDVWGQGTTVTVSS |
| SEQ ID NO: 3152 | VH | E-H.20 | EVQLVQSGAEVKKPGESLKISCKASGYAFSSSWMNWVRQMP |
| | | | GKGLEWIGRIYPGDGDTKYNGKFKGQATLSADKPISTAYLQ |
| | | | WSSLKASDTAMYYCARRGTGGWYFDVWGQGTTVTVSS |
| SEQ ID NO: 3153 | VH | E-H.21 | EVQLVQSGAEVKKPGESLRISCKASGYAFSSSWMNWVRQMP |
| | | | GKGLEWIGRIYPGDGDTKYNGKFKGQATLSADKSISTAYLQ |
| | | | WSSLKASDTAMYYCARRGTGGWYFDVWGQGTTVTVSS |
| SEQ ID NO: 3154 | VH | E-H.22 | EVQLVQSGAEVKKPGESLRISCKASGYAFSSSWMNWVRQMP |
| | | | GKGLEWIGRIYPGDGDTKYNGKFKGHATLSADKSISTAYLQ |
| | | | WSSLKASDTAMYYCARRGTGGWYFDVWGQGTTVTVSS |
| SEQ ID NO: 3155 | VH | E-H.23 | QVQLVQSGAEVKKTGSSVKVSCKASGYAFSSSWMNWVRQA |
| | | | PRQALEWIGRIYPGDGDTKYNGKFKGRATLTADKSMSTAYM |
| | | | ELSSLRSEDTAMYYCARRGTGGWYFDVWGQGTTVTVSS |
| SEQ ID NO: 3156 | VH | E-H.24 | EVQLVESGGGLVQPGRSLRLSCTASGYAFSSSWMNWVRQAP |
| | | | GKGLEWIGRIYPGDGDTKYNGKFKGRATLSADKSKSIAYLQ |
| | | | MNSLKTEDTAVYYCARRGTGGWYFDVWGQGTTVTVSS |
| SEQ ID NO: 3157 | VH | E-H.25 | EVQLVESGGGLVQPGPSLRLSCTASGYAFSSSWMNWVRQAP |
| | | | GKGLEWIGRIYPGDGDTKYNGKFKGRATLSADKSKSIAYLQ |
| | | | MNSLKTEDTAVYYCARRGTGGWYFDVWGQGTTVTVSS |
| SEQ ID NO: 3158 | VH | E-H.26 | QVQLQESGPGLVKPSQTLSLTCTASGYAFSSSWMNWVRQPP |
| | | | GKGLEWIGRIYPGDGDTKYNGKFKGRATLSADKSKSQASLK |
| | | | LSSVTAADTAVYYCARRGTGGWYFDVWGQGTTVTVSS |
| SEQ ID NO: 3159 | VH | E-H.27 | QVQLQESGPGLVKPSGTLSLTCAASGYAFSSSWMNWVRQPP |
| | | | GKGLEWIGRIYPGDGDTKYNGKFKGRATLSADKSKSQASLK |
| | | | LSSVTAADTAVYYCARRGTGGWYFDVWGQGTTVTVSS |
| SEQ ID NO: 3160 | VH | E-H.28 | EVQLVESGGGLVKPGRSLRLSCTASGYAFSSSWMNWVRQAP |
| | | | GKGLEWIGRIYPGDGDTKYNGKFKGRATLSADKSKSIAYLQ |
| | | | MNSLKTEDTAVYYCARRGTGGWYFDVWGQGTTVTVSS |
| SEQ ID NO: 3161 | VH | E-H.29 | EVQLVESGGGLVQPGGSLKLSCAASGYAFSSSWMNWVRQA |
| | | | SGKGLEWIGRIYPGDGDTKYNGKFKGRATLSADKSKSTAYL |
| | | | QMNSLKTEDTAVYYCARRGTGGWYFDVWGQGTTVTVSS |
| SEQ ID NO: 3162 | VH | E-H.30 | QVQLQESGPGLVKPSQTLSLTCAASGYAFSSSWMNWVRQPP |
| | | | GKGLEWIGRIYPGDGDTKYNGKFKGRATLSADKSKSQASLK |
| | | | LSSVTAADTAVYYCARRGTGGWYFDVWGQGTTVTVSS |
| SEQ ID NO: 3163 | VH | E-H.31 | EVQLVESGGGLVKPGGSLRLSCAASGYAFSSSWMNWVRQA |
| | | | PGKGLEWIGRIYPGDGDTKYNGKFKGRATLSADKSKSTAYL |
| | | | QMNSLKTEDTAVYYCARRGTGGWYFDVWGQGTTVTVSS |
| SEQ ID NO: 3164 | VH | E-H.32 | EVQLVESGGALVKPGGSLRLSCAASGYAFSSSWMNWVRQA |
| | | | PGKGLEWIGRIYPGDGDTKYNGKFKGRATLSADKSKSTAYL |
| | | | QMNSLKTEDTAVYYCARRGTGGWYFDVWGQGTTVTVSS |
| SEQ ID NO: 3165 | VH | E-H.33 | QVQLQESGPGLVKPSQTLSLTCAAYGYAFSSSWMNWVRQPP |
| | | | GKGLEWIGRIYPGDGDTKYNGKFKGRATLSADKSKSQASLK |
| | | | LSSVTAADTAVYYCARRGTGGWYFDVWGQGTTVTVSS |
| SEQ ID NO: 3166 | VH | E-H.34 | QVQLQESGSGLVKPSQTLSLTCAASGYAFSSSWMNWVRQPP |
| | | | GKGLEWIGRIYPGDGDTKYNGKFKGRATLSADKSKSQASLK |
| | | | LSSVTAADTAVYYCARRGTGGWYFDVWGQGTTVTVSS |
| SEQ ID NO: 3167 | VH | E-H.35 | EVQLVESGGGLVQPGGSLRLSCAASGYAFSSSWMNWVRQA |
| | | | PGKGLEWIGRIYPGDGDTKYNGKFKGRATLSADKSKSSAYL |
| | | | QMNSLKTEDTAVYYCARRGTGGWYFDVWGQGTTVTVSS |
| SEQ ID NO: 3168 | VH | E-H.36 | QVQLQESGPGLVKPSDTLSLTCTASGYAFSSSWMNWVRQPP |
| | | | GKGLEWIGRIYPGDGDTKYNGKFKGRATLSADKSKSQASLK |
| | | | LSSVTAADTAVYYCARRGTGGWYFDVWGQGTTVTVSS |
| SEQ ID NO: 3169 | VH | E-H.37 | QVQLQESGPGLVKPSQTLSLTCTASGYAFSSSWMNWVRQHP |
| | | | GKGLEWIGRIYPGDGDTKYNGKFKGRATLSADKSKSQASLK |
| | | | LSSVTAADTAVYYCARRGTGGWYFDVWGQGTTVTVSS |
| SEQ ID NO: 3170 | VH | E-H.38 | QVQLQESGPGLVKPSQTLSLTCTASGYAFSSSWMNWVRQHP |
| | | | GKGLEWIGRIYPGDGDTKYNGKFKGLATLSADKSKSQASLK |
| | | | LSSVTAADTAVYYCARRGTGGWYFDVWGQGTTVTVSS |
| SEQ ID NO: 3171 | VH | E-H.39 | QVQLVESGGGVVQPGRSLRLSCAASGYAFSSSWMNWVRQA |
| | | | PGKGLEWIGRIYPGDGDTKYNGKFKGRATLSADKSKSTAYL |
| | | | QMSSLRAEDTAVYYCARRGTGGWYFDVWGQGTTVTVSS |
| SEQ ID NO: 3172 | VH | E-H.40 | QVQLVESGGGLVKPGGSLRLSCAASGYAFSSSWMNWVRQA |
| | | | PGKGLEWIGRIYPGDGDTKYNGKFKGRATLSADKAKSSAYL |
| | | | QMNSLRAEDTAVYYCARRGTGGWYFDVWGQGTTVTVSS |
| SEQ ID NO: 3173 | VH | E-H.41 | QVQLVESGGGLVQPGGSLRLSCSASGYAFSSSWMNWVRQAP |
| | | | GKGLEWIGRIYPGDGDTKYNGKFKGRATLSADKSKSTAYLQ |
| | | | MNSLRAEDTAVYYCARRGTGGWYFDVWGQGTTVTVSS |
| SEQ ID NO: 3174 | VH | E-H.42 | QVQLLESGGGLVKPGGSLRLSCAASGYAFSSSWMNWVRQA |
| | | | PGKGLEWIGRIYPGDGDTKYNGKFKGRATLSADKAKSSAYL |
| | | | QMNSLRAEDTAVYYCARRGTGGWYFDVWGQGTTVTVSS |
| SEQ ID NO: 3175 | VH | E-H.43 | EVQLVESGGGLVQPGGSLRLSCSASGYAFSSSWMNWVRQAP |
| | | | GKGLEWIGRIYPGDGDTKYNGKFKGRATLSADKSKSTAYLQ |
| | | | MSSLRAEDTAVYYCARRGTGGWYFDVWGQGTTVTVSS |
| SEQ ID NO: 3176 | VH | E-H.44 | QVQLQESGPGLVKPSDTLSLTCAASGYAFSSSWMNWVRQPP |
| | | | GKGLEWIGRIYPGDGDTKYNGKFKGRATLSADKSKSQASLK |
| | | | LSSVTAVDTAVYYCARRGTGGWYFDVWGQGTTVTVSS |
| SEQ ID NO: 3177 | VH | E-H.45 | QVQLQESGPGLVKPSQTLSLTCAASGYAFSSSWMNWVRQPP |
| | | | GKGLEWIGRIYPGDGDTKYNGKFKGRATLSADKSKSQASLK |
| | | | LSSVTAVDTAVYYCARRGTGGWYFDVWGQGTTVTVSS |
| SEQ ID NO: 3178 | VH | E-H.46 | EVQLVESGGGLVQPGGSLRLSCSASGYAFSSSWMNWVRQAP |
| | | | GKGLEWIGRIYPGDGDTKYNGKFKGRATLSADKSKSTAYVQ |
| | | | MSSLRAEDTAVYYCARRGTGGWYFDVWGQGTTVTVSS |
| SEQ ID NO: 3179 | VH | E-H.47 | QVQLVDSGGGVVQPGRSLRLSCAASGYAFSSSWMNWVRQA |
| | | | PGKGLEWIGRIYPGDGDTKYNGKFKGRATLSADKSKSTAYL |
| | | | QMNSLRAEDTAVYYCARRGTGGWYFDVWGQGTTVTVSS |
| SEQ ID NO: 3180 | VH | E-H.48 | QVQLVESGGGVVQPGRSLRLSCAASGYAFSSSWMNWVRQA |
| | | | PGKGLEWIGRIYPGDGDTKYNGKFKGRATLSADKSKSTAYL |
| | | | QMNSLRAEGTAVYYCARRGTGGWYFDVWGQGTTVTVSS |
| SEQ ID NO: 3181 | VH | E-H.49 | QVQLVESGGGVVQPGRSLRLSCAASGYAFSSSWMNWVRQA |
| | | | PGKGLEWIGRIYPGDGDTKYNGKFKGRATLSADKSKSTAYL |
| | | | QMNSLRAEDTAVYYCARRGTGGWYFDVWGQGTTVTVSS |
| SEQ ID NO: 3182 | VH | E-H.50 | EVQLVESGGGLVQPGGSLRLSCAASGYAFSSSWMNWVRQA |
| | | | PGKGLEWIGRIYPGDGDTKYNGKFKGRATLSADKSKSTAYL |
| | | | QMNSLRAEDTAVYYCARRGTGGWYFDVWGQGTTVTVSS |

TABLE 12
Amino acid sequences for anti TCRβ V10 antibodies. Amino acid and nucleotide sequences for
murine and humanized antibody molecules which bind to TCRBV 10 (e.g., TCRBV 10-1, TCRBV 10-2 or
TCRBV 10-3). The amino acid of the heavy and light chain CDRs, and the amino acid and nucleotide
sequences of the heavy and light chain variable regions, and the heavy and light chains are shown.
Murine antibody D, also referred to as S511 antibody

| | | |
|---|---|---|
| SEQ ID NO: 1288 | HC CDR1 (Kabat) | SYGMS |
| SEQ ID NO: 1289 | HC CDR2 (Kabat) | LISSGGSYTYYTDSVKG |
| SEQ ID NO: 1290 | HC CDR3 (Kabat) | HGGNFFDY |
| SEQ ID NO: 1291 | HC CDR1 (Chothia) | GFTFRSY |
| SEQ ID NO: 1292 | HC CDR2 (Chothia) | SSGGSY |
| SEQ ID NO: 1290 | HC CDR3 (Chothia) | HGGNFFDY |
| SEQ ID NO: 1293 | HC CDR1 (Combined) | GFTFRSYGMS |
| SEQ ID NO: 1289 | HC CDR2 (Combined) | LISSGGSYTYYTDSVKG |
| SEQ ID NO: 1290 | HC CDR3(Combined) | HGGNFFDY |
| SEQ ID NO: 1294 | LC CDR1 (Kabat) | SVSSSVSYMH |
| SEQ ID NO: 1295 | LC CDR2 (Kabat) | DTSKLAS |
| SEQ ID NO: 1296 | LC CDR3 (Kabat) | QQWSSNPQYT |
| SEQ ID NO: 1297 | LC CDR1 (Chothia) | SSSVSY |
| SEQ ID NO: 1295 | LC CDR2 (Chothia) | DTSKLAS |
| SEQ ID NO: 1296 | LC CDR3 (Chothia) | QQWSSNPQYT |
| SEQ ID NO: 1294 | LC CDR1 (Combined) | SVSSSVSYMH |
| SEQ ID NO: 1295 | LC CDR2 (Combined) | DTSKLAS |
| SEQ ID NO: 1296 | LC CDR3 (Combined) | QQWSSNPQYT |
| SEQ ID NO: 3183 | VH | EVQLVESGGDLVKPGGSLKLSCAVSGFTFRSYGMSWVR |
| | | QTPDKRLEWVALISSGGSYTYYTDSVKGRFTISRDNAKN |
| | | TLYLQMSSLKSEDTAIYYCSRHGGNFFDYWGQGTTLTVS |
| | | S |
| SEQ ID NO: 3184 | VL | QIVLTQSPSIMSASPGEKVTMTCSVSSSVSYMHWYQQKS |
| | | GTSPKRWIYDTSKLASGVPARFSGSGSGTSYSLTISSMEA |
| | | EDAATYYCQQWSSNPQYTFGGGTKLEIK |

Variable light chain (VL)

| | | | |
|---|---|---|---|
| SEQ ID NO: 3185 | VL | D-H.1 | DIVLTQSPAFLSVTPGEKVTITCSVSSSVSYMHWYQQKPDQAPK |
| | | | LLIYDTSKLASGVPSRFSGSGSGTDYTFTISSLEAEDAATYYCQQ |
| | | | WSSNPQYTFGQGTKLEIK |
| SEQ ID NO: 3186 | VL | D-H.2 | AIQLTQSPSSLSASVGDRVTITCSVSSSVSYMHWYQQKPGKAPK |
| | | | LLIYDTSKLASGVPSRFSGSGSGTDYTLTISSLQPEDFATYYCQQ |
| | | | WSSNPQYTFGQGTKLEIK |
| SEQ ID NO: 3187 | VL | D-H.3 | DIQLTQSPSFLSASVGDRVTITCSVSSSVSYMHWYQQKPGKAPK |
| | | | LLIYDTSKLASGVPSRFSGSGSGTEYTLTISSLQPEDFATYYCQQ |
| | | | WSSNPQYTFGQGTKLEIK |
| SEQ ID NO: 3188 | VL | D-H.4 | DIQLTQSPSSLSASVGDRVTITCSVSSSVSYMHWYQQKPGKAPK |
| | | | LLIYDTSKLASGVPSRFSGSGSGTDYTLTISSLQPEDFATYYCQQ |
| | | | WSSNPQYTFGQGTKLEIK |
| SEQ ID NO: 3189 | VL | D-H.5 | DIQLTQSPSSVSASVGDRVTITCSVSSSVSYMHWYQQKPGKAPK |
| | | | LLIYDTSKLASGVPSRFSGSGSGTDYTLTISSLQPEDFATYYCQQ |
| | | | WSSNPQYTFGQGTKLEIK |
| SEQ ID NO: 3190 | VL | D-H.6 | DIQLTQSPSSLSASVGDRVTITCSVSSSVSYMHWYQQKPGKVPK |
| | | | LLIYDTSKLASGVPSRFSGSGSGTDYTLTISSLQPEDVATYYCQQ |
| | | | WSSNPQYTFGQGTKLEIK |
| SEQ ID NO: 3191 | VL | D-H.7 | DIQLTQSPSSLSASVGDRVTITCSVSSSVSYMHWYQQKPGQAPK |
| | | | LLIYDTSKLASGVPSRFSGSGSGTDYTLTISSLQPEDVATYYCQQ |
| | | | WSSNPQYTFGQGTKLEIK |
| SEQ ID NO: 3192 | VL | D-H.8 | EIVLTQSPDFQSVTPKEKVTITCSVSSSVSYMHWYQQKPDQSPKL |
| | | | LIYDTSKLASGVPSRFSGSGSGTDYTLTINSLEAEDAATYYCQQ |
| | | | WSSNPQYTFGQGTKLEIK |
| SEQ ID NO: 3193 | VL | D-H.9 | AIRLTQSPFSLSASVGDRVTITCSVSSSVSYMHWYQQKPAKAPK |
| | | | LFIYDTSKLASGVPSRFSGSGSGTDYTLTISSLQPEDFATYYCQQ |
| | | | WSSNPQYTFGQGTKLEIK |
| SEQ ID NO: 3194 | VL | D- | DIQLTQSPSSLSASVGDRVTITCSVSSSVSYMHWYQQKPGKAPK |
| | | H.10 | LLIYDTSKLASGVPSRFSGSGSGTDYTFTISSLQPEDIATYYCQQ |
| | | | WSSNPQYTFGQGTKLEIK |
| SEQ ID NO: 3195 | VL | D- | EIVLTQSPATLSLSPGERATLSCSVSSSVSYMHWYQQKPGQAPK |
| | | H.11 | LLIYDTSKLASGIPARFSGSGSGTDYTLTISSLEPEDFAVYYCQQ |
| | | | WSSNPQYTFGQGTKLEIK |
| SEQ ID NO: 3196 | VL | D- | DIQLTQSPSTLSASVGDRVTITCSVSSSVSYMHWYQQKPGKAPK |
| | | H.12 | LLIYDTSKLASGVPSRFSGSGSGTEYTLTISSLQPDDFATYYCQQ |
| | | | WSSNPQYTFGQGTKLEIK |
| SEQ ID NO: 3197 | VL | D- | DIQLTQSPSSLSASVGDRVTITCSVSSSVSYMHWYQQKPGKTPK |
| | | H.13 | LLIYDTSKLASGIPSRFSGSGSGTDYTLTIRSLQPEDFATYYCQQ |
| | | | WSSNPQYTFGQGTKLEIK |
| SEQ ID NO: 3198 | VL | D- | EIVLTQSPPTLSLSPGERVTLSCSVSSSVSYMHWYQQKPGQAPKL |
| | | H.14 | LIYDTSKLASGIPARFSGSGSGTDYTLTISSLQPEDFAVYYCQQW |
| | | | SSNPQYTFGQGTKLEIK |
| SEQ ID NO: 3199 | VL | D- | DIQLTQSPSSLSASVGDRVTITCSVSSSVSYMHWYQQKPGKAPK |
| | | H.15 | RLIYDTSKLASGVPSRFSGSGSGTEYTLTISSLQPEDFATYYCQQ |
| | | | WSSNPQYTFGQGTKLEIK |
| SEQ ID NO: 3200 | VL | D- | EIVLTQSPATLSLSPGERATLSCSVSSSVSYMHWYQQKPGQAPK |
| | | H.16 | LLIYDTSKLASGIPARFSGSGPGTDYTLTISSLEPEDFAVYYCQQ |
| | | | WSSNPQYTFGQGTKLEIK |
| SEQ ID NO: 3201 | VL | D- | EIVLTQSPATLSLSPGERATLSCSVSSSVSYMHWYQQKPGQAPK |
| | | H.17 | LLIYDTSKLASGIPARFSGSGSGTDYTLTISRLEPEDFAVYYCQQ |
| | | | WSSNPQYTFGQGTKLEIK |
| SEQ ID NO: 3202 | VL | D- | EIVLTQSPATLSLSPGERATLSCSVSSSVSYMHWYQQKPGQAPK |
| | | H.18 | LLIYDTSKLASGIPARFSGSGSGTDYTLTISSLQPEDFAVYYCQQ |
| | | | WSSNPQYTFGQGTKLEIK |
| SEQ ID NO: 3203 | VL | D- | EIVLTQSPATLSVSPGERATLSCSVSSSVSYMHWYQQKPGQAPK |
| | | H.19 | LLIYDTSKLASGIPARFSGSGSGTEYTLTISSLQSEDFAVYYCQQ |
| | | | WSSNPQYTFGQGTKLEIK |
| SEQ ID NO: 3204 | VL | D- | EIVLTQSPATLSVSPGERATLSCSVSSSVSYMHWYQQKPGQAPK |
| | | H.20 | LLIYDTSKLASGIPARFSGSGSGTEYTLTISILQSEDFAVYYCQQW |
| | | | SSNPQYTFGQGTKLEIK |
| SEQ ID NO: 3205 | VL | D- | EIVLTQSPPTLSLSPGERVTLSCSVSSSVSYMHWYQQKPGQAPKL |
| | | H.21 | LIYDTSKLASSIPARFSGSGSGTDYTLTISSLQPEDFAVYYCQQWS |
| | | | SNPQYTFGQGTKLEIK |
| SEQ ID NO: 3206 | VL | D- | DIQLTQSPSSLSASVGDRVTITCSVSSSVSYMHWYQQKPGKAPK |
| | | H.22 | SLIYDTSKLASGVPSRFSGSGSGTDYTLTISSLQPEDFATYYCQQ |
| | | | WSSNPQYTFGQGTKLEIK |
| SEQ ID NO: 3207 | VL | D. | DIQLTQSPSSLSASVGDRVTITCSVSSSVSYMHWYQQKPGKAPK |
| | | H.23 | RLIYDTSKLASGVPSRFSGSGSGTEYTLTISNLQPEDFATYYCQQ |
| | | | WSSNPQYTFGQGTKLEIK |
| SEQ ID NO: 3208 | VL | D- | DIQLTQSPSAMSASVGDRVTITCSVSSSVSYMHWYQQKPGKVPK |
| | | H.24 | RLIYDTSKLASGVPSRFSGSGSGTEYTLTISSLQPEDFATYYCQQ |
| | | | WSSNPQYTFGQGTKLEIK |
| SEQ ID NO: 3209 | VL | D- | EIVLTQSPATLSLSPGERATLSCSVSSSVSYMHWYQQKPGQAPK |
| | | H.25 | LLIYDTSKLASGIPDRFSGSGSGTDYTLTISRLEPEDFAVYYCQQ |
| | | | WSSNPQYTFGQGTKLEIK |
| SEQ ID NO: 3210 | VL | D- | EIVLTQSPATLSLSPGERATLSCSVSSSVSYMHWYQQKPGLAPKL |
| | | H.26 | LIYDTSKLASGIPDRFSGSGSGTDYTLTISRLEPEDFAVYYCQQW |
| | | | SSNPQYTFGQGTKLEIK |
| SEQ ID NO: 3211 | VL | D- | EIVLTQSPGTLSLSPGERATLSCSVSSSVSYMHWYQQKPGQAPK |
| | | H.27 | LLIYDTSKLASGIPDRFSGSGSGTDYTLTISRLEPEDFAVYYCQQ |
| | | | WSSNPQYTFGQGTKLEIK |
| SEQ ID NO: 3212 | VL | D- | DIQLTQSPSSLSASVGDRVTITCSVSSSVSYMHWYQQKPGKAPK |
| | | H.28 | SLIYDTSKLASGVPSKFSGSGSGTDYTLTISSLQPEDFATYYCQQ |
| | | | WSSNPQYTFGQGTKLEIK |
| SEQ ID NO: 3213 | VL | D- | DIQLTQSPSSLSASVGDRVTITCSVSSSVSYMHWYQQKPEKAPKS |
| | | H.29 | LIYDTSKLASGVPSRFSGSGSGTDYTLTISSLQPEDFATYYCQQW |
| | | | SSNPQYTFGQGTKLEIK |
| SEQ ID NO: 3214 | VL | D- | DIVLTQSPDSLAVSLGERATINCSVSSSVSYMHWYQQKPGQPPK |
| | | H.30 | LLIYDTSKLASGVPDRFSGSGSGTDYTLTISSLQAEDVAVYYCQQ |
| | | | WSSNPQYTFGQGTKLEIK |
| SEQ ID NO: 3215 | VL | D- | EIVLTQTPLSLSITPGEQASMSCSVSSSVSYMHWYLQKARPVPKL |
| | | H.31 | LIYDTSKLASGVPDRFSGSGSGTDYTLKISRVEAEDFGVYYCQQ |
| | | | WSSNPQYTFGQGTKLEIK |
| SEQ ID NO: 3216 | VL | D- | EIVLTQTPLSLSITPGEQASISCSVSSSVSYMHWYLQKARPVPKLL |
| | | H.32 | IYDTSKLASGVPDRFSGSGSGTDYTLKISRVEAEDFGVYYCQQW |
| | | | SSNPQYTFGQGTKLEIK |
| SEQ ID NO: 3217 | VL | D- | DIVLTQSPLSLPVTPGEPASISCSVSSSVSYMHWYLQKPGQSPKL |
| | | H.33 | LIYDTSKLASGVPDRFSGSGSGTDYTLKISRVEAEDVGVYYCQQ |
| | | | WSSNPQYTFGQGTKLEIK |
| SEQ ID NO: 3218 | VL | D- | DIVLTQSPLSLPVTLGQPASISCSVSSSVSYMHWYQQRPGQSPKR |
| | | H.34 | LIYDTSKLASGVPDRFSGSGSGTDYTLKISRVEAEDVGVYYCQQ |
| | | | WSSNPQYTFGQGTKLEIK |
| SEQ ID NO: 3219 | VL | D- | DIVLTQTPLSLPVTPGEPASISCSVSSSVSYMHWYLQKPGQSPKL |
| | | H.35 | LIYDTSKLASGVPDRFSGSGSGTDYTLKISRVEAEDVGVYYCQQ |
| | | | WSSNPQYTFGQGTKLEIK |
| SEQ ID NO: 3220 | VL | D- | DIVLTQTPLSLSVTPGQPASISCSVSSSVSYMHWYLQKPGQSPKL |
| | | H.36 | LIYDTSKLASGVPDRFSGSGSGTDYTLKISRVEAEDVGVYYCQQ |
| | | | WSSNPQYTFGQGTKLEIK |
| SEQ ID NO: 3221 | VL | D- | DIVLTQTPLSLSVTPGQPASISCSVSSSVSYMHWYLQKPGQPPKL |
| | | H.37 | LIYDTSKLASGVPDRFSGSGSGTDYTLKISRVEAEDVGVYYCQQ |
| | | | WSSNPQYTFGQGTKLEIK |
| SEQ ID NO: 3222 | VL | D- | DIQLIQSPSFLSASVGDRVSIICSVSSSVSYMHWYLQKPGKSPKLF |
| | | H.38 | IYDTSKLASGVSSRFSGRGSGTDYTLTIISLKPEDFAAYYCQQWS |
| | | | SNPQYTFGQGTKLEIK |
| SEQ ID NO: 3223 | VL | D- | DIVLTQTPLSSPVTLGQPASISCSVSSSVSYMHWYQQRPGQPPKL |
| | | H.39 | LIYDTSKLASGVPDRFSGSGAGTDYTLKISRVEAEDVGVYYCQQ |
| | | | WSSNPQYTFGQGTKLEIK |
| SEQ ID NO: 3224 | VL | D- | EITLTQSPAFMSATPGDKVNISCSVSSSVSYMHWYQQKPGEAPK |
| | | H.40 | FIIYDTSKLASGIPPRFSGSGYGTDYTLTINNIESEDAAYYYCQQW |
| | | | SSNPQYTFGQGTKLEIK |

Variable HEAVY chain (VH)

| | | | |
|---|---|---|---|
| SEQ ID NO: 3225 | VH | D-H.1 | EVQLVESGGGLVKPGGSLRLSCAVSGFTFRSYGMSWVRQAPGK |
| | | | GLEWVALISSGGSYTYYTDSVKGRFTISRDNSKNTLYLQMNSLK |
| | | | TEDTAVYYCSRHGGNFFDYWGQGTTVTVSS |
| SEQ ID NO: 3226 | VH | D-H.2 | EVQLVESGGALVKPGGSLRLSCAVSGFTFRSYGMSWVRQAPGK |
| | | | GLEWVALISSGGSYTYYTDSVKGRFTISRDNSKNTLYLQMNSLK |
| | | | TEDTAVYYCSRHGGNFFDYWGQGTTVTVSS |
| SEQ ID NO: 3227 | VH | D-H.3 | EVQLVESGGGLVQPGGSLRLSCAVSGFTFRSYGMSWVRQAPGK |
| | | | GLEWVALISSGGSYTYYTDSVKGRFTISRDNAKNTLYLQMNSLR |
| | | | AEDTAVYYCSRHGGNFFDYWGQGTTVTVSS |
| SEQ ID NO: 3228 | VH | D-H.4 | EVQLVESGGGLVQPGGSLRLSCAVSGFTFRSYGMSWVRQAPGK |
| | | | GLEWVALISSGGSYTYYTDSVKGRFTISRDNAKNSLYLQMNSLR |
| | | | AEDTAVYYCSRHGGNFFDYWGQGTTVTVSS |
| SEQ ID NO: 3229 | VH | D-H.5 | EVQLVESGGGLVQPGGSLRLSCAVSGFTFRSYGMSWVRQAPGK |
| | | | GLEWVALISSGGSYTYYTDSVKGRFTISRDNSKNSLYLQMNSLK |
| | | | TEDTAVYYCSRHGGNFFDYWGQGTTVTVSS |
| SEQ ID NO: 3230 | VH | D-H.6 | EVQLVESGGGLVQPGGSLRLSCAVSGFTFRSYGMSWVRQAPGK |
| | | | GLEWVALISSGGSYTYYTDSVKGRFTISRDNAKNSLYLQMNSLR |
| | | | AEDMAVYYCSRHGGNFFDYWGQGTTVTVSS |
| SEQ ID NO: 3231 | VH | D-H.7 | EVQLVESGGGLVQPGGSLRLSCAVSGFTFRSYGMSWVRQAPGK |
| | | | GLEWVALISSGGSYTYYTDSVKGQFTISRDNAKNTLYLQMNSLR |
| | | | AEDMAVYYCSRHGGNFFDYWGQGTTVTVSS |
| SEQ ID NO: 3232 | VH | D-H.8 | EVQLVESGGGLVKPGRSLRLSCTVSGFTFRSYGMSWVRQAPGK |
| | | | GLEWVALISSGGSYTYYTDSVKGRFTISRDNSKNILYLQMNSLK |
| | | | TEDTAVYYCSRHGGNFFDYWGQGTTVTVSS |
| SEQ ID NO: 3233 | VH | D-H.9 | EVQLVESGGGLVKPGGSLRLSCAVSGFTFRSYGMSWVRQAPGK |
| | | | GLEWVALISSGGSYTYYTDSVKGRFTISRDNAKNSLYLQMNSLR |
| | | | AEDTAVYYCSRHGGNFFDYWGQGTTVTVSS |
| SEQ ID NO: 3234 | VH | D- | EVQLVESGGGLVQPGGSLKLSCAVSGFTFRSYGMSWVRQASGK |
| | | H.10 | GLEWVALISSGGSYTYYTDSVKGRFTISRDNSKNTLYLQMNSLK |
| | | | TEDTAVYYCSRHGGNFFDYWGQGTTVTVSS |
| SEQ ID NO: 3235 | VH | D- | QVQLVESGGGVVQPGGSLRLSCAVSGFTFRSYGMSWVRQAPGK |
| | | H.11 | GLEWVALISSGGSYTYYTDSVKGRFTISRDNSKNTLYLQMNSLR |
| | | | AEDTAVYYCSRHGGNFFDYWGQGTTVTVSS |
| SEQ ID NO: 3236 | VH | D- | QVQLVESGGGVVQPGRSLRLSCAVSGFTFRSYGMSWVRQAPGK |
| | | H.12 | GLEWVALISSGGSYTYYTDSVKGRFTISRDNSKNTLYLQMSSLR |
| | | | AEDTAVYYCSRHGGNFFDYWGQGTTVTVSS |
| SEQ ID NO: 3237 | VH | D- | EVQLVESGGGLVQPGGSLRLSCPVSGFTFRSYGMSWVRQAPGK |
| | | H.13 | GLEWVALISSGGSYTYYTDSVKGRFTISRDNANNSLYLQMNSLR |
| | | | AEDTAVYYCSRHGGNFFDYWGQGTTVTVSS |
| SEQ ID NO: 3238 | VH | D- | EVQLVESGGGLVQPGRSLRLSCTVSGFTFRSYGMSWVRQAPGK |
| | | H.14 | GLEWVALISSGGSYTYYTDSVKGRFTISRDNSKNILYLQMNSLK |
| | | | TEDTAVYYCSRHGGNFFDYWGQGTTVTVSS |
| SEQ ID NO: 3239 | VH | D- | EVQLVESGGGLVQPGPSLRLSCTVSGFTFRSYGMSWVRQAPGK |
| | | H.15 | GLEWVALISSGGSYTYYTDSVKGRFTISRDNSKNILYLQMNSLK |
| | | | TEDTAVYYCSRHGGNFFDYWGQGTTVTVSS |
| SEQ ID NO: 3240 | VH | D- | EVQLVESGGGLVQPGGSLRLSCAVSGFTFRSYGMSWVRQAPGK |
| | | H.16 | GLEWVALISSGGSYTYYTDSVKGRFTISRDNSKNTLYLQMNSLR |
| | | | AEDTAVYYCSRHGGNFFDYWGQGTTVTVSS |
| SEQ ID NO: 3241 | VH | D- | EVQLVESGGGLVQPGGSLRLSCAVSGFTFRSYGMSWVRQAPGK |
| | | H.17 | GLEWVALISSGGSYTYYTDSVKGRFTISRDNAKNSLYLQMNSLR |
| | | | DEDTAVYYCSRHGGNFFDYWGQGTTVTVSS |
| SEQ ID NO: 3242 | VH | D- | QVQLVESGGGLVKPGGSLRLSCAVSGFTFRSYGMSWVRQAPGK |
| | | H.18 | GLEWVALISSGGSYTYYTDSVKGRFTISRDNAKNSLYLQMNSLR |
| | | | AEDTAVYYCSRHGGNFFDYWGQGTTVTVSS |
| SEQ ID NO: 3243 | VH | D- | QVQLVESGGGVVQPGRSLRLSCAVSGFTFRSYGMSWVRQAPGK |
| | | H.19 | GLEWVALISSGGSYTYYTDSVKGRFTISRDNSKNTLYLQMNSLR |
| | | | AEDTAVYYCSRHGGNFFDYWGQGTTVTVSS |
| SEQ ID NO: 3244 | VH | D- | EVQLLESGGGLVQPGGSLRLSCAVSGFTFRSYGMSWVRQAPGK |
| | | H.20 | GLEWVALISSGGSYTYYTDSVKGRFTISRDNSKNTLYLQMNSLR |
| | | | AEDTAVYYCSRHGGNFFDYWGQGTTVTVSS |
| SEQ ID NO: 3245 | VH | D- | EVQLVESGGGLVQPGGSLRLSCAVSGFTFRSYGMSWVRQAPGK |
| | | H.21 | GLEWVALISSGGSYTYYTDSVKGRFTISRHNSKNTLYLQMNSLR |
| | | | AEDTAVYYCSRHGGNFFDYWGQGTTVTVSS |
| SEQ ID NO: 3246 | VH | D- | EVQLVESGGGLIQPGGSLRLSCAVSGFTFRSYGMSWVRQPPGKG |
| | | H.22 | LEWVALISSGGSYTYYTDSVKGRFTISRDNSKNTLYLQMNSLRA |
| | | | EDTAVYYCSRHGGNFFDYWGQGTTVTVSS |
| SEQ ID NO: 3247 | VH | D- | EVQLVESGGGLIQPGGSLRLSCAVSGFTFRSYGMSWVRQAPGK |
| | | H.23 | GLEWVALISSGGSYTYYTDSVKGRFTISRDNSKNTLYLQMNSLR |
| | | | AEDTAVYYCSRHGGNFFDYWGQGTTVTVSS |
| SEQ ID NO: 3248 | VH | D- | EVQLVESGGGLVQPGRSLRLSCAVSGFTFRSYGMSWVRQAPGK |
| | | H.24 | GLEWVALISSGGSYTYYTDSVKGRFTISRDNAKNSLYLQMNSLR |
| | | | AEDTALYYCSRHGGNFFDYWGQGTTVTVSS |
| SEQ ID NO: 3249 | VH | D- | QVQLVESGGGVVQPGRSLRLSCAVSGFTFRSYGMSWVRQAPGK |
| | | H.25 | GLEWVALISSGGSYTYYTDSVKGRFTISRDNSKNRLYLQMNSLR |
| | | | AEDTAVYYCSRHGGNFFDYWGQGTTVTVSS |
| SEQ ID NO: 3250 | VH | D- | QVQLVESGGGVVQPGRSLRLSCAVSGFTFRSYGMSWVRQAPGK |
| | | H.26 | GLEWVALISSGGSYTYYTDSVKGRFTISRDNSKNTLYLQMNSLR |
| | | | AEGTAVYYCSRHGGNFFDYWGQGTTVTVSS |
| SEQ ID NO: 3251 | VH | D- | QVQLVESGGGVVQPGRSLRLSCAVSGFTFRSYGMSWVRQAPGK |
| | | H.27 | GLEWVALISSGGSYTYYTDSVKGRFAISRDNSKNTLYLQMNSLR |
| | | | AEDTAVYYCSRHGGNFFDYWGQGTTVTVSS |
| SEQ ID NO: 3252 | VH | D- | QVQLVDSGGGVVQPGRSLRLSCAVSGFTFRSYGMSWVRQAPG |
| | | H.28 | KGLEWVALISSGGSYTYYTDSVKGRFTISRDNSKNTLYLQMNSL |
| | | | RAEDTAVYYCSRHGGNFFDYWGQGTTVTVSS |
| SEQ ID NO: 3253 | VH | D- | EVQLVESGGGVVRPGGSLRLSCAVSGFTFRSYGMSWVRQAPGK |
| | | H.29 | GLEWVALISSGGSYTYYTDSVKGRFTISRDNAKNSLYLQMNSLR |
| | | | AEDTALYHCSRHGGNFFDYWGQGTTVTVSS |
| SEQ ID NO: 3254 | VH | D- | EVQLVESGGVVVQPGGSLRLSCAVSGFTFRSYGMSWVRQAPGK |
| | | H.30 | GLEWVALISSGGSYTYYTDSVKGRFTISRDNSKNSLYLQMNSLR |
| | | | AEDTALYYCSRHGGNFFDYWGQGTTVTVSS |
| SEQ ID NO: 3255 | VH | D- | EVQLVESGGGVVQPGGSLRLSCAVSGFTFRSYGMSWVRQAPGK |
| | | H.31 | GLEWVALISSGGSYTYYTDSVKGRFTISRDNSKNSLYLQMNSLR |
| | | | TEDTALYYCSRHGGNFFDYWGQGTTVTVSS |
| SEQ ID NO: 3256 | VH | D- | EVQLVESGGVVVQPGGSLRLSCAVSGFTFRSYGMSWVRQAPGK |
| | | H.32 | GLEWVALISSGGSYTYYTDSVKGRFTISRDNSKNSLYLQMNSLR |
| | | | TEDTALYYCSRHGGNFFDYWGQGTTVTVSS |
| SEQ ID NO: 3257 | VH | D- | EVQLVETGGGLIQPGGSLRLSCAVSGFTFRSYGMSWVRQAPGK |
| | | H.33 | GLEWVALISSGGSYTYYTDSVKGRFTISRDNSKNTLYLQMNSLR |
| | | | AEDTAVYYCSRHGGNFFDYWGQGTTVTVSS |
| SEQ ID NO: 3258 | VH | D- | EVQLVESGGGLVQPGGSLRLSCAVSGFTFRSYGMSWVRQATGK |
| | | H.34 | GLEWVALISSGGSYTYYTDSVKGRFTISRENAKNSLYLQMNSLR |
| | | | AGDTAVYYCSRHGGNFFDYWGQGTTVTVSS |
| SEQ ID NO: 3259 | VH | D- | EVQLVESRGVLVQPGGSLRLSCAVSGFTFRSYGMSWVRQAPGK |
| | | H.35 | GLEWVALISSGGSYTYYTDSVKGRFTISRDNSKNTLHLQMNSLR |
| | | | AEDTAVYYCSRHGGNFFDYWGQGTTVTVSS |
| SEQ ID NO: 3260 | VH | D- | EVQLVESGGGLVQPGRSLRLSCAVSGFTFRSYGMSWVRQAPGK |
| | | H.36 | GLEWVALISSGGSYTYYTDSVKGRFTISRDNAKNSLYLQMNSLR |
| | | | AEDMALYYCSRHGGNFFDYWGQGTTVTVSS |
| SEQ ID NO: 3261 | VH | D- | QVQLVESGGGLVQPGGSLRLSCSVSGFTFRSYGMSWVRQAPGK |
| | | H.37 | GLEWVALISSGGSYTYYTDSVKGRFTISRDNSKNTLYLQMNSLR |
| | | | AEDTAVYYCSRHGGNFFDYWGQGTTVTVSS |
| SEQ ID NO: 3262 | VH | D- | EVQLVESGGGLVQPGGSLRLSCSVSGFTFRSYGMSWVRQAPGK |
| | | H.38 | GLEWVALISSGGSYTYYTDSVKGRFTISRDNSKNTLYLQMSSLR |
| | | | AEDTAVYYCSRHGGNFFDYWGQGTTVTVSS |
| SEQ ID NO: 3263 | VH | D- | QVQLVESGGGVVQPGRSLRLSCAVSGFTFRSYGMSWVRQAPGK |
| | | H.39 | GLEWVALISSGGSYTYYTDSVKGRFTISRDNSTNTLFLQMNSLR |
| | | | AEDTAVYYCSRHGGNFFDYWGQGTTVTVSS |
| SEQ ID NO: 3264 | VH | D- | QVQLLESGGGLVKPGGSLRLSCAVSGFTFRSYGMSWVRQAPGK |
| | | H.40 | GLEWVALISSGGSYTYYTDSVKGRFTISRDNAKNSLYLQMNSLR |
| | | | AEDTAVYYCSRHGGNFFDYWGQGTTVTVSS |
| SEQ ID NO: 3265 | VH | D- | EVQLVESGEGLVQPGGSLRLSCAVSGFTFRSYGMSWVRQAPGK |
| | | H.41 | GLEWVALISSGGSYTYYTDSVKGRFTISRDNSKNTLYLQMGSLR |
| | | | AEDMAVYYCSRHGGNFFDYWGQGTTVTVSS |
| SEQ ID NO: 3266 | VH | D- | EVQLVESGGGLVQPGGSLRLSCAVSGFTFRSYGMSWVRQAPGK |
| | | H.42 | GLEWVALISSGGSYTYYTDSVKGRFTISRDNSKNTLYLQMGSLR |
| | | | AEDMAVYYCSRHGGNFFDYWGQGTTVTVSS |
| SEQ ID NO: 3267 | VH | D- | EVQLVESGGGLVQPGGSLRLSCSVSGFTFRSYGMSWVRQAPGK |
| | | H.43 | GLEWVALISSGGSYTYYTDSVKGRFTISRDNSKNTLYVQMSSLR |
| | | | AEDTAVYYCSRHGGNFFDYWGQGTTVTVSS |
| SEQ ID NO: 3268 | VH | D- | EVQLVESGGGLVQPGGSLRLSCAVSGFTFRSYGMSWVRQAPGK |
| | | H.44 | GLEWVALISSGGSYTYYTDSVKGRFIISRDNSRNSLYLQKNRRR |
| | | | AEDMAVYYCSRHGGNFFDYWGQGTTVTVSS |
| SEQ ID NO: 3269 | VH | D- | EVQLVESGGGLVQPGGSLRLSCAVSGFTFRSYGMSWVHQAPGK |
| | | H.45 | GLEWVALISSGGSYTYYTDSVKGRFIISRDNSRNTLYLQTNSLRA |
| | | | EDTAVYYCSRHGGNFFDYWGQGTTVTVSS |
| SEQ ID NO: 3270 | VH | D- | EVHLVESGGGLVQPGGALRLSCAVSGFTFRSYGMSWVRQATGK |
| | | H.46 | GLEWVALISSGGSYTYYTDSVKGRFTISRENAKNSLYLQMNSLR |
| | | | AGDTAVYYCSRHGGNFFDYWGQGTTVTVSS |
| SEQ ID NO: 3271 | VH | D- | EVQLVESGGGLVQPRGSLRLSCAVSGFTFRSYGMSWVRQAPGK |
| | | H.47 | GLEWVALISSGGSYTYYTDSVKGRFTISRDNSKNTLYLQMNNLR |
| | | | AEGTAVYYCSRHGGNFFDYWGQGTTVTVSS |
| SEQ ID NO: 3272 | VH | D- | EVQLVESGGGLVQPRGSLRLSCAVSGFTFRSYGMSWVRQAPGK |
| | | H.48 | GLEWVALISSGGSYTYYTDSVKGRFTISRDNSKNTLYLQMNNLR |
| | | | AEGTAAYYCSRHGGNFFDYWGQGTTVTVSS |
| SEQ ID NO: 3273 | VH | D- | QVQLVQSGAEVKKPGASVKVSCKVSGFTFRSYGMSWVRQAPG |
| | | H.49 | KGLEWVALISSGGSYTYYTDSVKGRFTITRDNSTNTLYMELSSL |
| | | | RSEDTAVYYCSRHGGNFFDYWGQGTTVTVSS |
| SEQ ID NO: 3274 | VH | D- | QVQLVQSGSELKKPGASVKVSCKVSGFTFRSYGMSWVRQAPGQ |
| | | H.50 | GLEWVALISSGGSYTYYTDSVKGRFVISRDNSVNTLYLQISSLKA |
| | | | EDTAVYYCSRHGGNFFDYWGQGTTVTVSS |

TABLE 13
Amino acid sequences for additional TCRβV antibodies. Amino acid and nucleotide sequences
for murine and humanized antibody molecules which bind to various TCRVB families are
disclosed. The amino acid the heavy and light chain CDRs, and the amino acid and nucleotide
sequences of the heavy and light chain variable regions, and the heavy and light chains are
shown. Antibodies disclosed in the tablei nclude, IMMU222 and JOVI-3. IMMU 222 binds human
TCRβV 6-5, TCRβV 6-6, or TCRβV 6-9 (TCRβV13.1 per old nomenclature). JOVI-3 binds human
TCRβV 28 (TCRβV3.1 per old nomenclature).
IMMU222 (murine) also referred to as BJ1461; or Antibody I
Binds to human TCRVβ 6-5, 6-6, 6-9

| | | |
|---|---|---|
| SEQ ID NO: 1141 | HC CDR1 (Kabat) | SYAMS |
| SEQ ID NO: 1142 | HC CDR2 (Kabat) | HISNGGDYIYYADTVKG |
| SEQ ID NO: 1143 | HC CDR3 (Kabat) | PSYYSDPWFFDV |
| SEQ ID NO: 1144 | HC CDR1 (Chothia) | GFTFRSY |
| SEQ ID NO: 1145 | HC CDR2 (Chothia) | SNGGDY |
| SEQ ID NO: 1143 | HC CDR3 (Chothia) | PSYYSDPWFFDV |
| SEQ ID NO: 1146 | HC CDR1 (Combined) | GFTFRSYAMS |
| SEQ ID NO: 1142 | HC CDR2 (Combined) | HISNGGDYIYYADTVKG |
| SEQ ID NO: 1143 | HC CDR3 (Combined) | PSYYSDPWFFDV |
| SEQ ID NO: 1147 | LC CDR1 (Kabat) | SAGSSVSFMH |
| SEQ ID NO: 1148 | LC CDR2 (Kabat) | DTSKLAS |
| SEQ ID NO: 1149 | LC CDR3 (Kabat) | LQGSGFPLT |
| SEQ ID NO: 1150 | LC CDR1 (Chothia) | GSSVSF |
| SEQ ID NO: 1148 | LC CDR2 (Chothia) | DTSKLAS |
| SEQ ID NO: 1149 | LC CDR3 (Chothia) | LQGSGFPLT |
| SEQ ID NO: 1147 | LC CDR1 (Combined) | SAGSSVSFMH |
| SEQ ID NO: 1148 | LC CDR2 (Combined) | DTSKLAS |
| SEQ ID NO: 1149 | LC CDR3 (Combined) | LQGSGFPLT |
| SEQ ID NO: 1151 | VL | ENVLTQSPAIMSASPGEKVTMTCSAGSSVSFMHWYQQKSSTSP |
| | | KLWIYDTSKLASGVPGRFSGSGSGNSFSLTISSMEAEDVAIYYC |
| | | LQGSGFPLTFGSGTKLEIK |
| SEQ ID NO: 1152 | VH | DVKLVESGEGLVKPGGSLKLSCAASGFTFRSYAMSWVRQTPE |
| | | KRLEWVAHISNGGDYIYYADTVKGRFTISRDNARNTLYLQMS |
| | | SLKSEDTAMYYCTRPSYYSDPWFFDVWGTGTTVTVSS |

VH for IMMU222 (humanized)) also referred to as Antibody I-H
Binds to human TCRVβ 6-5, 6-6, 6-9

| | | |
|---|---|---|
| SEQ ID NO: 1153 | VH-1 | EVQLVESGGGLVQPGGSLRLSCAASGFTFRSYAMSWVRQAPG |
| | | KGLEWVAHISNGGDYIYYADTVKGRFTISRDNAKNSLYLQMN |
| | | SLRAEDTAVYYCTRPSYYSDPWFFDVWGQGTTVTVSS |
| SEQ ID NO: 1154 | VH-2 | QVQLVESGGGVVQPGRSLRLSCAASGFTFRSYAMSWVRQAPG |
| | | KGLEWVAHISNGGDYIYYADTVKGRFTISRDNSKNTLYLQMS |
| | | SLRAEDTAVYYCTRPSYYSDPWFFDVWGQGTTVTVSS |
| SEQ ID NO: 1155 | VH-3 | EVQLVESGGGLVQPGGSLRLSCAASGFTFRSYAMSWVRQAPG |
| | | KGLEWVAHISNGGDYIYYADTVKGRFTISRDNSKNTLYLQMN |
| | | SLRAEDTAVYYCTRPSYYSDPWFFDVWGQGTTVTVSS |
| SEQ ID NO: 1156 | VH-4 | QVQLVQSGSELKKPGASVKVSCKASGFTFRSYAMSWVRQAPG |
| | | QGLEWVAHISNGGDYIYYADTVKGRFVISRDNSVNTLYLQISS |
| | | LKAEDTAVYYCTRPSYYSDPWFFDVWGQGTTVTVSS |
| SEQ ID NO: 1157 | VH-5 | QVQLVQSGAEVKKPGASVKVSCKASGFTFRSYAMSWVRQAP |
| | | GQRLEWVAHISNGGDYIYYADTVKGRFTITRDNSANTLYMEL |
| | | SSLRSEDTAVYYCTRPSYYSDPWFFDVWGQGTTVTVSS |

VL for IMMU222 (humanized)) also referred to as Antibody I-H
Binds to human TCRVβ 6-5, 6-6, 6-9

| | | |
|---|---|---|
| SEQ ID NO: 1158 | VL-1 | ENVLTQSPATLSLSPGERATLSCSAGSSVSFMHWYQQKPGQAP |
| | | KLLIYDTSKLASGIPARFSGSGSGNDFTLTISSLEPEDFAVYYCL |
| | | QGSGFPLTFGQGTKLEIK |
| SEQ ID NO: 1159 | VL-2 | ENVLTQSPDFQSVTPKEKVTITCSAGSSVSFMHWYQQKPDQSP |
| | | KLLIYDTSKLASGVPSRFSGSGSGNDFTLTINSLEAEDAATYYC |
| | | LQGSGFPLTFGQGTKLEIK |
| SEQ ID NO: 1160 | VL-3 | DNQLTQSPSSLSASVGDRVTITCSAGSSVSFMHWYQQKPGKVP |
| | | KLLIYDTSKLASGVPSRFSGSGSGNDFTLTISSLQPEDVATYYC |
| | | LQGSGFPLTFGQGTKLEIK |
| SEQ ID NO: 1161 | VL-4 | ANQLTQSPSSLSASVGDRVTITCSAGSSVSFMHWYQQKPGKAP |
| | | KLLIYDTSKLASGVPSRFSGSGSGNDFTLTISSLQPEDFATYYCL |
| | | QGSGFPLTFGQGTKLEIK |
| SEQ ID NO: 1162 | VL-5 | DNVLTQSPDSLAVSLGERATINCSAGSSVSFMHWYQQKPGQPP |
| | | KLLIYDTSKLASGVPDRFSGSGSGNDFTLTISSLQAEDVAVYYC |
| | | LQGSGFPLTFGQGTKLEIK |

JOVI-3 (murine), also referred to as BJ1187 or Antibody K
Binds to human TCRVβ 28

| | | |
|---|---|---|
| SEQ ID NO: 1185 | HC CDR1 (Kabat) | GSWMN |
| SEQ ID NO: 1186 | HC CDR2 (Kabat) | RIYPGDGDTDYSGKFKG |
| SEQ ID NO: 1187 | HC CDR3 (Kabat) | SGYFNYVPVFDY |
| SEQ ID NO: 1188 | HC CDR1 (Chothia) | GYTFSGS |
| SEQ ID NO: 1189 | HC CDR2 (Chothia) | YPGDGD |
| SEQ ID NO: 1187 | HC CDR3 (Chothia) | SGYFNYVPVFDY |
| SEQ ID NO: 1190 | HC CDR1 (Combined) | GYTFSGSWMN |
| SEQ ID NO: 1186 | HC CDR2 (Combined) | RIYPGDGDTDYSGKFKG |
| SEQ ID NO: 1187 | HC CDR3 (Combined) | SGYFNYVPVFDY |
| SEQ ID NO: 1191 | LC CDR1 (Kabat) | SANSTVGYIH |
| SEQ ID NO: 1192 | LC CDR2 (Kabat) | TTSNLAS |
| SEQ ID NO: 1193 | LC CDR3 (Kabat) | HQWSFYPT |
| SEQ ID NO: 1194 | LC CDR1 (Chothia) | NSTVGY |
| SEQ ID NO: 1192 | LC CDR2 (Chothia) | TTSNLAS |
| SEQ ID NO: 1193 | LC CDR3 (Chothia) | HQWSFYPT |
| SEQ ID NO: 1191 | LC CDR1 (Combined) | SANSTVGYIH |
| SEQ ID NO: 1192 | LC CDR2 (Combined) | TTSNLAS |
| SEQ ID NO: 1193 | LC CDR3 (Combined) | HQWSFYPT |
| SEQ ID NO: 1195 | VL | QIVLTQSPAIMSASLGEEIALTCSANSTVGYIHWYQQKSGTSPK |
| | | LLIYTTSNLASGVPSRFSGSGSGTFYSLTISSVEAEDAADYFCH |
| | | QWSFYPTFGGGTKLEIK |
| SEQ ID NO: 1196 | VH | QIQLQQSGPEVVKPGASVQISCKASGYTFSGSWMNWVKQRPG |
| | | KGLEWIGRIYPGDGDTDYSGKFKGRATLTADKSSSTAYMRLSS |
| | | LTSEDSAVYFCARSGYFNYVPVFDYWGQGTTLSVSS |

VH for JOVI-3 (humanized) also referred to as Antibody K-H
Binds to human TCRVβ 28

| | | |
|---|---|---|
| SEQ ID NO: 1197 | VH-1 | QIQLVQSGAEVKKPGASVKVSCKASGYTFSGSWMNWVRQAP |
| | | GQGLEWIGRIYPGDGDTDYSGKFKGRATLTADKSTSTAYMEL |
| | | SSLRSEDTAVYYCARSGYFNYVPVFDYWGQGTTVTVSS |
| SEQ ID NO: 1198 | VH-2 | QIQLVQSGAEVKKPGSSVKVSCKASGYTFSGSWMNWVRQAP |
| | | GQGLEWIGRIYPGDGDTDYSGKFKGRATLTADKSTSTAYMEL |
| | | SSLRSEDTAVYYCARSGYFNYVPVFDYWGQGTTVTVSS |
| SEQ ID NO: 1199 | VH-3 | EIQLVQSGAEVKKPGESLKISCKASGYTFSGSWMNWVRQMPG |
| | | KGLEWIGRIYPGDGDTDYSGKFKGQATLSADKSISTAYLQWSS |
| | | LKASDTAMYYCARSGYFNYVPVFDYWGQGTTVTVSS |
| SEQ ID NO: 1200 | VH-4 | QIQLVQSGSELKKPGASVKVSCKASGYTFSGSWMNWVRQAP |
| | | GQGLEWIGRIYPGDGDTDYSGKFKGRAVLSADKSVSTAYLQIS |
| | | SLKAEDTAVYYCARSGYFNYVPVFDYWGQGTTVTVSS |
| SEQ ID NO: 1201 | VH-5 | QIQLVQSGSELKKPGASVKVSCKASGYTFSGSWMNWVRQAP |
| | | GQGLEWIGRIYPGDGDTDYSGKFKGRAVLSADKSVSMAYLQI |
| | | SSLKAEDTAVYYCARSGYFNYVPVFDYWGQGTTVTVSS |
| SEQ ID NO: 1202 | VH-6 | EIQLVESGGGLVQPGRSLRLSCTASGYTFSGSWMNWVRQAPG |
| | | KGLEWIGRIYPGDGDTDYSGKFKGRATLSADKSKSIAYLQMN |
| | | SLKTEDTAVYYCARSGYFNYVPVFDYWGQGTTVTVSS |

VL for JOVI-3 (humanized) also referred to as Antibody K-H
Binds to human TCRVβ 28

| | | |
|---|---|---|
| SEQ ID NO: 1203 | VL-1 | EIVLTQSPATLSLSPGERATLSCSANSTVGYIHWYQQKPGQAP |
| | | KLLIYTTSNLASGIPARFSGSGSGTDYTLTISSLEPEDFAVYFCH |
| | | QWSFYPTFGQGTKLEIK |
| SEQ ID NO: 1204 | VL-2 | DIQLTQSPSFLSASVGDRVTITCSANSTVGYIHWYQQKPGKAP |
| | | KLLIYTTSNLASGVPSRFSGSGSGTEYTLTISSLQPEDFATYFCH |
| | | QWSFYPTFGQGTKLEIK |
| SEQ ID NO: 1205 | VL-3 | EIVLTQSPATLSLSPGERATLSCSANSTVGYIHWYQQKPGQAP |
| | | KLLIYTTSNLASGIPARFSGSGPGTDYTLTISSLEPEDFAVYFCH |
| | | QWSFYPTFGQGTKLEIK |
| SEQ ID NO: 1206 | VL-4 | DIVLTQSPDSLAVSLGERATINCSANSTVGYIHWYQQKPGQPP |
| | | KLLIYTTSNLASGVPDRFSGSGSGTDYTLTISSLQAEDVAVYFC |
| | | HQWSFYPTFGQGTKLEIK |
| SEQ ID NO: 1207 | VL-5 | EIVLTQSPDFQSVTPKEKVTITCSANSTVGYIHWYQQKPDQSPK |
| | | LLIYTTSNLASGVPSRFSGSGSGTDYTLTINSLEAEDAATYFCH |
| | | QWSFYPTFGQGTKLEIK |

TABLE 14
Exemplary Fc modifications

| Modification or mutation | Altered effector function |
|---|---|
| Leu235Glu | ADCC; |
| Leu234Ala/Leu235Ala (LALA) | ADCC; ADCP; CDC |
| Ser228Pro/Leu235Glu | |
| Leu234Ala/Leu235Ala/Pro329Gly | ADCP |
| Pro331Ser/Leu234Glu/Leu235Phe | CDC |
| Asp265Ala | ADCC, ADCP |
| Gly237Ala | ADCP |
| Glu318Ala | ADCP |
| Glu233Pro | |
| Gly236Arg/Leu328Arg | ADCC |
| His268Gln/Val309Leu/Ala330Ser/Pro331Ser | ADCC; ADCP; CDC |
| Val234Ala/Gly237Ala/Pro238Ser/ | ADCC; ADCP; CDC |
| His268Ala/Val309Leu/Ala330Ser/Pro331Ser | |
| Leu234Ala/L235Ala/Gly237Ala/P238Ser/ | ADCC; CDC |
| His268Ala/Ala330Ser/Pro331Ser | |
| Ala330Leu | CDC |
| Asp270Ala | CDC |
| Lys322Ala | CDC |
| Pro329Ala | CDC |
| Pro331Ala | CDC |
| Val264Ala | CDC |
| High mannose glycosylation | CDC |
| Phe241Ala | CDC |
| Asn297Ala or Gly or Gln | ADCC; ADCP; CDC |
| S228P/Phe234Ala/Leu235Ala | ADCC; CDC |

TABLE 15
Exemplary agents that comprise a first binder that binds to a first target molecule, wherein
the exemplary first binder comprises anti-TCRβ V20 antibodies. Exemplary first target
molecule comprises TCRVB 20 (e.g., TCRVB 20-1). Exemplary anti-TCR V20 antibodies include,
but are not limited to, BLM0274 and BLM0276, which binds to human TCRβV 20-1. CDRs are
underlined.
BLM0274, also referred to as BJ1325 or BJ1329
Binds to human TCRVβ 20-1

| | | |
|---|---|---|
| SEQ ID NO: 3318 | Heavy chain (VH-CH) | QVQLVQSGAEVKKPGASVKVSCKASGFNIKSAYMHWVRQA |
| | | PGQEPGCMGRIDPATGKTKYAPKFQARVTMTADTSINTAYTE |
| | | LSSLRSEDTATYYCARSLNWDYGLDYWGQGTLVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHKP |
| | | SNTKVDKRVEPKSCDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVCTLPPSREEMTKNQVSLSCAVKGFYP |
| | | SDIAVEWESNGQPENNYKTTPPVLDSDGSFFLVSKLTVDKSR |
| | | WQQGNVFSCSVMHEALHNHYTQKSLSLSPGK |
| SEQ ID NO: 3319 | Light chain (VL-CL) | EIVLTQSPATLSLSPGERATLSCRASKSVSILGTHLIHWYQQKP |
| | | GLAPRLLIYAASNLESGIPDRFSGSGSETDFTLTISRLEPEDFAV |
| | | YFCQQSIEDPWTFGGGTKVEIKRTVAAPSVFIFPPSDEQLKSG |
| | | TASVVCLLNNFYPREAKVQWKVDNALQSGNSQESVTEQDSK |
| | | DSTYSLSSTLTLSKADYEKHKVYACEVTHQGLSSPVTKSFNR |
| | | GEC |
| SEQ ID NO: 3340 | IL-2 C125A-Heavy | APTSSSTKKTQLQLEHLLLDLQMILNGINNYKNPKLTRMLTF |
| | Chain Constant | KFYMPKKATELKHLQCLEEELKPLEEVLNLAQSKNFHLRPRD |
| | Domain (Fc Region) | LISNINVIVLELKGSETTFMCEYADETATIVEFLNRWITFAQSII |
| | | STLTGGGGSGGGGSDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVYTLPPCREEMTKNQVSLWCLVKGFY |
| | | PSDIAVEWESNGQPENNYKTTPPVLDSDGSFFLYSKLTVDKS |
| | | RWQQGNVFSCSVMHEALHNHYTQKSLSLSPGK |
| SEQ ID NO: 3321 | HC CDR1 | GFNIKSAYMH |
| SEQ ID NO: 3322 | HC CDR2 | RIDPATGKTKYAPKFQA |
| SEQ ID NO: 3323 | HC CDR3 | SLNWDYGLDY |
| SEQ ID NO: 3324 | LC CDR1 | RASKSVSILGTHLIH |
| SEQ ID NO: 3325 | LC CDR2 | AASNLES |
| SEQ ID NO: 3326 | LC CDR3 | QQSIEDPWT |
| SEQ ID NO: 3448 | VH | QVQLVQSGAEVKKPGASVKVSCKASGFNIKSAYMHWVRQAP |
| | | GQEPGCMGRIDPATGKTKYAPKFQARVTMTADTSINTAYTEL |
| | | SSLRSEDTATYYCARSLNWDYGLDYWGQGTLVTVSS |
| SEQ ID NO: 3449 | VL | EIVLTQSPATLSLSPGERATLSCRASKSVSILGTHLIHWYQQKP |
| | | GLAPRLLIYAASNLESGIPDRFSGSGSETDFTLTISRLEPEDFAV |
| | | YFCQQSIEDPWTFGGGTKVEIK |
| SEQ ID NO: 3649 | Heavy chain constant | ASTKGPSVFPLAPSSKSTSGGTAALGCLVKDYFPEPVTVSWNS |
| | region (CH1-CH2-CH3) | GALTSGVHTFPAVLQSSGLYSLSSVVTVPSSSLGTQTYICNVNH |
| | Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSPGK |
| SEQ ID NO: 3447 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSPGK |
| SEQ ID NO: 3644 | Light chain constant | RTVAAPSVFIFPPSDEQLKSGTASVVCLLNNFYPREAKVQWKV |
| | region | DNALQSGNSQESVTEQDSKDSTYSLSSTLTLSKADYEKHKVY |
| | | ACEVTHQGLSSPVTKSFNRGEC |
| SEQ ID NO: 2270 | IL-2 C125A | APTSSSTKKTQLQLEHLLLDLQMILNGINNYKNPKLTRMLTFK |
| | | FYMPKKATELKHLQCLEEELKPLEEVLNLAQSKNFHLRPRDLI |
| | | SNINVIVLELKGSETTFMCEYADETATIVEFLNRWITFAQSIIST |
| | | LT |
| SEQ ID NO: 3308 | Linker | GGGGSGGGGS |
| SEQ ID NO: 3648 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSPGK |

BLM0276, also referred to as BJ1324, BJ1330
Binds to human TCRVβ 20-1

| | | |
|---|---|---|
| SEQ ID NO: 3328 | Heavy chain (VH-CH) | QVQLVQSGAEVKKPGASVKVSCKASGFNIKSAYMHWVRQA |
| | | PGQGLEWMGRIDPATGKTKYAPKFQARVTMTADTSTNTAY |
| | | MELSSLRSEDTAVYYCARSLNWDYGLDYWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVNH |
| | | KPSNTKVDKRVEPKSCDKTHTCPPCPAPELLGGPSVFLFPPKP |
| | | KDTLMISRTPEVTCVVVDVSHEDPEVKFNWYVDGVEVHNAK |
| | | TKPREEQYASTYRVVSVLTVLHQDWLNGKEYKCKVSNKALP |
| | | APIEKTISKAKGQPREPQVCTLPPSREEMTKNQVSLSCAVKGF |
| | | YPSDIAVEWESNGQPENNYKTTPPVLDSDGSFFLVSKLTVDK |
| | | SRWQQGNVFSCSVMHEALHNHYTQKSLSLSPGK |
| SEQ ID NO: 3329 | Light chain (VL-CL) | EIVLTQSPGTLSLSPGERATLSCRASKSVSILGTHLIHWYQQKP |
| | | GQAPRLLIYAASNLESGIPDRFSGSGSETDFTLTISRLEPEDFA |
| | | VYFCQQSIEDPWTFGGGTKVEIKRTVAAPSVFIFPPSDEQLKS |
| | | GTASVVCLLNNFYPREAKVQWKVDNALQSGNSQESVTEQDS |
| | | KDSTYSLSSTLTLSKADYEKHKVYACEVTHQGLSSPVTKSFN |
| | | RGEC |
| SEQ ID NO: 3340 | IL-2 C125A-Heavy | APTSSSTKKTQLQLEHLLLDLQMILNGINNYKNPKLTRMLTF |
| | Chain Constant | KFYMPKKATELKHLQCLEEELKPLEEVLNLAQSKNFHLRPRD |
| | Domain (Fc Region) | LISNINVIVLELKGSETTFMCEYADETATIVEFLNRWITFAQSII |
| | | STLTGGGGSGGGGSDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVYTLPPCREEMTKNQVSLWCLVKGFY |
| | | PSDIAVEWESNGQPENNYKTTPPVLDSDGSFFLYSKLTVDKS |
| | | RWQQGNVFSCSVMHEALHNHYTQKSLSLSPGK |
| SEQ ID NO: 3321 | HC CDR1 | GFNIKSAYMH |
| SEQ ID NO: 3322 | HC CDR2 | RIDPATGKTKYAPKFQA |
| SEQ ID NO: 3323 | HC CDR3 | SLNWDYGLDY |
| SEQ ID NO: 3324 | LC CDR1 | RASKSVSILGTHLIH |
| SEQ ID NO: 3325 | LC CDR2 | AASNLES |
| SEQ ID NO: 3326 | LC CDR3 | QQSIEDPWT |
| SEQ ID NO: 3450 | VH | QVQLVQSGAEVKKPGASVKVSCKASGFNIKSAYMHWVRQAP |
| | | GQGLEWMGRIDPATGKTKYAPKFQARVTMTADTSTNTAYME |
| | | LSSLRSEDTAVYYCARSLNWDYGLDYWGQGTLVTVSS |
| SEQ ID NO: 3451 | VL | EIVLTQSPGTLSLSPGERATLSCRASKSVSILGTHLIHWYQQKP |
| | | GQAPRLLIYAASNLESGIPDRFSGSGSETDFTLTISRLEPEDFAV |
| | | YFCQQSIEDPWTFGGGTKVEIK |
| SEQ ID NO: 3649 | Heavy chain constant | ASTKGPSVFPLAPSSKSTSGGTAALGCLVKDYFPEPVTVSWNS |
| | region (CH1-CH2-CH3) | GALTSGVHTFPAVLQSSGLYSLSSVVTVPSSSLGTQTYICNVNH |
| | Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSPGK |
| SEQ ID NO: 3447 | Heavy chain hinge-Fc | DKTHTCPPCPAPELLGGPSVFLFPPKPKDTLMISRTPEVTCVVV |
| | region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSPGK |
| SEQ ID NO: 3644 | Light chain constant | RTVAAPSVFIFPPSDEQLKSGTASVVCLLNNFYPREAKVQWKV |
| | region | DNALQSGNSQESVTEQDSKDSTYSLSSTLTLSKADYEKHKVY |
| | | ACEVTHQGLSSPVTKSFNRGEC |
| SEQ ID NO: 2270 | IL-2 C125A | APTSSSTKKTQLQLEHLLLDLQMILNGINNYKNPKLTRMLTFK |
| | | FYMPKKATELKHLQCLEEELKPLEEVLNLAQSKNFHLRPRDLI |
| | | SNINVIVLELKGSETTFMCEYADETATIVEFLNRWITFAQSIIST |
| | | LT |
| SEQ ID NO: 3308 | Linker | GGGGSGGGGS |
| SEQ ID NO: 3648 | Heavy chain hinge-Fc | DKTHTCPPCPAPELLGGPSVFLFPPKPKDTLMISRTPEVTCVVV |
| | region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSPGK |

BLM0000

| | | |
|---|---|---|
| SEQ ID NO: 3328 | Heavy chain (VH-CH) | QVQLVQSGAEVKKPGASVKVSCKASGFNIKSAYMHWVRQA |
| | | PGQGLEWMGRIDPATGKTKYAPKFQARVTMTADTSTNTAY |
| | | MELSSLRSEDTAVYYCARSLNWDYGLDYWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVNH |
| | | KPSNTKVDKRVEPKSCDKTHTCPPCPAPELLGGPSVFLFPPKP |
| | | KDTLMISRTPEVTCVVVDVSHEDPEVKFNWYVDGVEVHNAK |
| | | TKPREEQYASTYRVVSVLTVLHQDWLNGKEYKCKVSNKALP |
| | | APIEKTISKAKGQPREPQVCTLPPSREEMTKNQVSLSCAVKGF |
| | | YPSDIAVEWESNGQPENNYKTTPPVLDSDGSFFLVSKLTVDK |
| | | SRWQQGNVFSCSVMHEALHNHYTQKSLSLSPGK |
| SEQ ID NO: 3329 | Light chain (VL-CL) | EIVLTQSPGTLSLSPGERATLSCRASKSVSILGTHLIHWYQQKP |
| | | GQAPRLLIYAASNLESGIPDRFSGSGSETDFTLTISRLEPEDFA |
| | | VYFCQQSIEDPWTFGGGTKVEIKRTVAAPSVFIFPPSDEQLKS |
| | | GTASVVCLLNNFYPREAKVQWKVDNALQSGNSQESVTEQDS |
| | | KDSTYSLSSTLTLSKADYEKHKVYACEVTHQGLSSPVTKSFN |
| | | RGEC |
| SEQ ID NO: 3350 | IL-2 C125A-Heavy | APTSSSTKKTQLQLEHLLLDLQMILNGINNYKNPKLTRMLTF |
| | Chain Constant | KFYMPKKATELKHLQCLEEELKPLEEVLNLAQSKNFHLRPRD |
| | Domain (Fc Region) | LISNINVIVLELKGSETTFMCEYADETATIVEFLNRWITFAQSII |
| | | STLTGGGGSGGGGSDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVYTLPPCREEMTKNQVSLWCLVKGFY |
| | | PSDIAVEWESNGQPENNYKTTPPVLDSDGSFFLYSKLTVDKS |
| | | RWQQGNVFSCSVMHEALHNHYTQKSLSLSP |
| SEQ ID NO: 3500 | IL-2 C125A-Heavy | APTSSSTKKTQLQLEHLLLDLQMILNGINNYKNPKLTRMLTF |
| | Chain Constant | KFYMPKKATELKHLQCLEEELKPLEEVLNLAQSKNFHLRPRD |
| | Domain (Fc Region) | LISNINVIVLELKGSETTFMCEYADETATIVEFLNRWITFAQSII |
| | | STLTGGGGSGGGGSDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVYTLPPCREEMTKNQVSLWCLVKGFY |
| | | PSDIAVEWESNGQPENNYKTTPPVLDSDGSFFLYSKLTVDKS |
| | | RWQQGNVFSCSVMHEALHNHYTQKSLSLSPGK |
| SEQ ID NO: 3321 | HC CDR1 | GFNIKSAYMH |
| SEQ ID NO: 3322 | HC CDR2 | RIDPATGKTKYAPKFQA |
| SEQ ID NO: 3323 | HC CDR3 | SLNWDYGLDY |
| SEQ ID NO: 3324 | LC CDR1 | RASKSVSILGTHLIH |
| SEQ ID NO: 3325 | LC CDR2 | AASNLES |
| SEQ ID NO: 3326 | LC CDR3 | QQSIEDPWT |
| SEQ ID NO: 3450 | VH | QVQLVQSGAEVKKPGASVKVSCKASGFNIKSAYMHWVRQAP |
| | | GQGLEWMGRIDPATGKTKYAPKFQARVTMTADTSTNTAYME |
| | | LSSLRSEDTAVYYCARSLNWDYGLDYWGQGTLVTVSS |
| SEQ ID NO: 3451 | VL | EIVLTQSPGTLSLSPGERATLSCRASKSVSILGTHLIHWYQQKP |
| | | GQAPRLLIYAASNLESGIPDRFSGSGSETDFTLTISRLEPEDFAV |
| | | YFCQQSIEDPWTFGGGTKVEIK |
| SEQ ID NO: 3649 | Heavy chain constant | ASTKGPSVFPLAPSSKSTSGGTAALGCLVKDYFPEPVTVSWNS |
| | region (CH1-CH2-CH3) | GALTSGVHTFPAVLQSSGLYSLSSVVTVPSSSLGTQTYICNVNH |
| | Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSPGK |
| SEQ ID NO: 3447 | Heavy chain hinge-Fc | DKTHTCPPCPAPELLGGPSVFLFPPKPKDTLMISRTPEVTCVVV |
| | region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSPGK |
| SEQ ID NO: 3644 | Light chain constant | RTVAAPSVFIFPPSDEQLKSGTASVVCLLNNFYPREAKVQWKV |
| | region | DNALQSGNSQESVTEQDSKDSTYSLSSTLTLSKADYEKHKVY |
| | | ACEVTHQGLSSPVTKSFNRGEC |
| SEQ ID NO: 2270 | IL-2 C12SA | APTSSSTKKTQLQLEHLLLDLQMILNGINNYKNPKLTRMLTFK |
| | | FYMPKKATELKHLQCLEEELKPLEEVLNLAQSKNFHLRPRDLI |
| | | SNINVIVLELKGSETTFMCEYADETATIVEFLNRWITFAQSIIST |
| | | LT |
| SEQ ID NO: 3308 | Linker | GGGGSGGGGS |
| SEQ ID NO: 3454 | Heavy chain hinge-Fc | DKTHTCPPCPAPELLGGPSVFLFPPKPKDTLMISRTPEVTCVVV |
| | region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSP |
| SEQ ID NO: 3501 | Heavy chain hinge-Fc | DKTHTCPPCPAPELLGGPSVFLFPPKPKDTLMISRTPEVTCVVV |
| | region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSPGK |

TABLE 16
Exemplary agents that comprise a first binder that binds to a first target molecule, wherein
the exemplary first binder comprises anti-TCRβ V6 antibodies. Exemplary first target
molecule comprises TCRVB 6 (e.g., TCRVB 6-5). Exemplary anti-TCRβ V6 antibodies include,
but are not limited to, BKM0186, which binds to human TCRβV 6-5. CDRs are underlined.
BKM0186, also referred to as BK141 or BK104
Binds to human TCRVβ 6-5

| | | |
|---|---|---|
| SEQ ID NO: 3348 | Heavy chain (VH-CH) | QVQLVQSGAEVKKPGSSVKVSCKASGHDFRLTYIHWVRQAP |
| | | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHKP |
| | | SNTKVDKRVEPKSCDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVCTLPPSREEMTKNQVSLSCAVKGFYP |
| | | SDIAVEWESNGQPENNYKTTPPVLDSDGSFFLVSKLTVDKSR |
| | | WQQGNVFSCSVMHEALHNHYTQKSLSLSP |
| SEQ ID NO: 3502 | Heavy chain (VH-CH) | QVQLVQSGAEVKKPGSSVKVSCKASGHDFRLTYIHWVRQAP |
| | | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHKP |
| | | SNTKVDKRVEPKSCDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVCTLPPSREEMTKNQVSLSCAVKGFYP |
| | | SDIAVEWESNGQPENNYKTTPPVLDSDGSFFLVSKLTVDKSR |
| | | WQQGNVFSCSVMHEALHNHYTQKSLSLSPGK |
| SEQ ID NO: 3349 | Light chain (VL-CL) | DIQMTQSPSFLSASVGDRVTITCKASQNVADRVVWHQQKPG |
| | | KAPKALIYSSSHRYKGVPSRFSGSGSGTEFTLTISSLQPEDFAT |
| | | YFCQQFKSYPLTFGQGTKLEIKRTVAAPSVFIFPPSDEQLKSGT |
| | | ASVVCLLNNFYPREAKVQWKVDNALQSGNSQESVTEQDSKD |
| | | STYSLSSTLTLSKADYEKHKVYACEVTHQGLSSPVTKSFNRG |
| | | EC |
| SEQ ID NO: 3350 | IL-2 C125A-Heavy | APTSSSTKKTQLQLEHLLLDLQMILNGINNYKNPKLTRMLTF |
| | Chain Constant | KFYMPKKATELKHLQCLEEELKPLEEVLNLAQSKNFHLRPRD |
| | Domain (Fc Region) | LISNINVIVLELKGSETTFMCEYADETATIVEFLNRWITFAQSII |
| | | STLTGGGGSGGGGSDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVYTLPPCREEMTKNQVSLWCLVKGFY |
| | | PSDIAVEWESNGQPENNYKTTPPVLDSDGSFFLYSKLTVDKS |
| | | RWQQGNVFSCSVMHEALHNHYTQKSLSLSP |
| SEQ ID NO: 3503 | IL-2 C125A-Heavy | APTSSSTKKTQLQLEHLLLDLQMILNGINNYKNPKLTRMLTF |
| | Chain Constant | KFYMPKKATELKHLQCLEEELKPLEEVLNLAQSKNFHLRPRD |
| | Domain (Fc Region) | LISNINVIVLELKGSETTFMCEYADETATIVEFLNRWITFAQSII |
| | | STLTGGGGGGGGSDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVYTLPPCREEMTKNQVSLWCLVKGFY |
| | | PSDIAVEWESNGQPENNYKTTPPVLDSDGSFFLYSKLTVDKS |
| | | RWQQGNVFSCSVMHEALHNHYTQKSLSLSPGK |
| SEQ ID NO: 3351 | HC CDR1 | GHDFRLTYIH |
| SEQ ID NO: 3352 | HC CDR2 | RVSAGSGNVKYNEKFKG |
| SEQ ID NO: 3353 | HC CDR3 | SYYSYDVLDY |
| SEQ ID NO: 3354 | LC CDR1 | KASQNVADRVV |
| SEQ ID NO: 3355 | LC CDR2 | SSSHRYK |
| SEQ ID NO: 3356 | LC CDR3 | QQFKSYPLT |
| SEQ ID NO: 1346 | VH | QVQLVQSGAEVKKPGSSVKVSCKASGHDERLTYIHWVRQAPG |
| | | QGLEWMGRVSAGSGNVKYNEKFKGRVTITADTSTSTAYMELS |
| | | SLRSEDTAVYYCAVSYYSYDVLDYWGQGTTVTVSS |
| SEQ ID NO: 1349 | VL | DIQMTQSPSFLSASVGDRVTITCKASQNVADRVVWHQQKPGK |
| | | APKALIYSSSHRYKGVPSRFSGSGSGTEFTLTISSLQPEDFATYF |
| | | CQQFKSYPLTFGQGTKLEIK |
| SEQ ID NO: 3452 | Heavy chain constant | ASTKGPSVFPLAPSSKSTSGGTAALGCLVKDYFPEPVTVSWNS |
| | region (CH1-CH2-CH3) | GALTSGVHTFPAVLQSSGLYSLSSVVTVPSSSLGTQTYICNVNH |
| | Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSP |
| SEQ ID NO: 3504 | Heavy chain constant | ASTKGPSVFPLAPSSKSTSGGTAALGCLVKDYFPEPVTVSWNS |
| | region (CH1-CH2-CH3) | GALTSGVHTFPAVLQSSGLYSLSSVVTVPSSSLGTQTYICNVNH |
| | Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSPGK |
| SEQ ID NO: 3453 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSP |
| SEQ ID NO: 3505 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSPGK |
| SEQ ID NO: 3644 | Light chain constant | RTVAAPSVFIFPPSDEQLKSGTASVVCLLNNFYPREAKVQWKV |
| | region | DNALQSGNSQESVTEQDSKDSTYSLSSTLTLSKADYEKHKVY |
| | | ACEVTHQGLSSPVTKSFNRGEC |
| SEQ ID NO: 2270 | IL-2 C125A | APTSSSTKKTQLQLEHLLLDLQMILNGINNYKNPKLTRMLTFK |
| | | FYMPKKATELKHLQCLEEELKPLEEVLNLAQSKNFHLRPRDLI |
| | | SNINVIVLELKGSETTFMCEYADETATIVEFLNRWITFAQSIIST |
| | | LT |
| SEQ ID NO: 3308 | Linker | GGGGSGGGGS |
| SEQ ID NO: 3454 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSP |
| SEQ ID NO: 3501 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSPGK |

TABLE 17
Exemplary agents that comprise a first binder that binds to a first target molecule,
wherein the exemplary first binder comprises anti-TCRβ V28 antibodies. Exemplary first
target molecule comprises TCRβ V28. Exemplary anti-TCRβ V28 antibodies include, but are
not limited to, BLM0286, which binds to human TCRβV 28. CDRs are underlined.
BLM0286, also referred to as BL203 or BJ1319
Binds to human TCRVβ 28

| | | |
|---|---|---|
| SEQ ID NO: 3358 | Heavy chain (VH-CH) | QIQLQQSGPEVVKPGASVQISCKASGYTFSGSWMNWVKQRP |
| | | GKGLEWIGRIYPGDGDTDYSGKFKGRATLTADKSSSTAYMR |
| | | LSSLTSEDSAVYFCARSGYFNYVPVFDYWGQGTTLSVSSAST |
| | | KGPSVFPLAPSSKSTSGGTAALGCLVKDYFPEPVTVSWNSGA |
| | | LTSGVHTFPAVLQSSGLYSLSSVVTVPSSSLGTQTYICNVNHK |
| | | PSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFY |
| | | PSDIAVEWESNGQPENNYKTTPPVLDSDGSFFLVSKLTVDKS |
| | | RWQQGNVFSCSVMHEALHNHYTQKSLSLSPGK |
| SEQ ID NO: 3359 | Light chain (VL-CL) | QIVLTQSPAIMSASLGEEIALTCSANSTVGYIHWYQQKSGTSP |
| | | KLLIYTTSNLASGVPSRFSGSGSGTFYSLTISSVEAEDAADYFC |
| | | HQWSFYPTFGGGTKLEIKRTVAAPSVFIFPPSDEQLKSGTASV |
| | | VCLLNNFYPREAKVQWKVDNALQSGNSQESVTEQDSKDSTY |
| | | SLSSTLTLSKADYEKHKVYACEVTHQGLSSPVTKSFNRGEC |
| SEQ ID NO: 3340 | IL-2 C125A-Heavy | APTSSSTKKTQLQLEHLLLDLQMILNGINNYKNPKLTRMLTF |
| | Chain Constant | KFYMPKKATELKHLQCLEEELKPLEEVLNLAQSKNFHLRPRD |
| | Domain (Fc Region) | LISNINVIVLELKGSETTFMCEYADETATIVEFLNRWITFAQSII |
| | | STLTGGGGSGGGGSDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVYTLPPCREEMTKNQVSLWCLVKGFY |
| | | PSDIAVEWESNGQPENNYKTTPPVLDSDGSFFLYSKLTVDKS |
| | | RWQQGNVFSCSVMHEALHNHYTQKSLSLSPGK |
| SEQ ID NO: 1190 | HC CDR1 | GYTFSGSWMN |
| SEQ ID NO: 1186 | HC CDR2 | RIYPGDGDTDYSGKFKG |
| SEQ ID NO: 1187 | HC CDR3 | SGYFNYVPVFDY |
| SEQ ID NO: 1191 | LC CDR1 | SANSTVGYIH |
| SEQ ID NO: 1192 | LC CDR2 | TTSNLAS |
| SEQ ID NO: 1193 | LC CDR3 | HQWSFYPT |
| SEQ ID NO: 1196 | VH | QIQLQQSGPEVVKPGASVQISCKASGYTFSGSWMNWVKQRPG |
| | | KGLEWIGRIYPGDGDTDYSGKFKGRATLTADKSSSTAYMRLSS |
| | | LTSEDSAVYFCARSGYFNYVPVFDYWGQGTTLSVSS |
| SEQ ID NO: 1195 | VL | QIVLTQSPAIMSASLGEEIALTCSANSTVGYIHWYQQKSGTSPK |
| | | LLIYTTSNLASGVPSRFSGSGSGTFYSLTISSVEAEDAADYFCH |
| | | QWSFYPTFGGGTKLEIK |
| SEQ ID NO: 3649 | Heavy chain constant | ASTKGPSVFPLAPSSKSTSGGTAALGCLVKDYFPEPVTVSWNS |
| | region (CH1-CH2-CH3) | GALTSGVHTFPAVLQSSGLYSLSSVVTVPSSSLGTQTYICNVNH |
| | Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSPGK |
| SEQ ID NO: 3447 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSPGK |
| SEQ ID NO: 3644 | Light chain constant | RTVAAPSVFIFPPSDEQLKSGTASVVCLLNNFYPREAKVQWKV |
| | region | DNALQSGNSQESVTEQDSKDSTYSLSSTLTLSKADYEKHKVY |
| | | ACEVTHQGLSSPVTKSFNRGEC |
| SEQ ID NO: 2270 | IL-2 C125A | APTSSSTKKTQLQLEHLLLDLQMILNGINNYKNPKLTRMLTFK |
| | | FYMPKKATELKHLQCLEEELKPLEEVLNLAQSKNFHLRPRDLI |
| | | SNINVIVLELKGSETTFMCEYADETATIVEFLNRWITFAQSIIST |
| | | LT |
| SEQ ID NO: 3308 | Linker | GGGGSGGGGS |
| SEQ ID NO: 3648 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSPGK |

TABLE 18
Exemplary agents that comprise a first binder that binds to a first target molecule,
wherein the exemplary first binder comprises anti-TCRβV19 antibodies. Exemplary first
target molecule comprises TCRVβ 19. Exemplary anti-TCRβV19 antibodies include, but are not
limited to, BLM0281, which binds to human TCRβV19. CDRs are underlined.
BLM0281, also referred to as BL195 or BJ1597
Binds to human TCRVβ 19

| | | |
|---|---|---|
| SEQ ID NO: 3368 | Heavy chain (VH-CH) | NVQLQESGPGLVKPSQSLSLTCSVAGYSITSGYFWNWIRQFP |
| | | GNKLEWMGYISYDGSNNYNPSLKNRISITRDTSKNQFFLKLN |
| | | SVTTEDTATYYCASPSPGTGYAVDYWGQGTSVTVSSASTKG |
| | | PSVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKRVEPKSCDKTHTCPPCPAPELLGGPSVFLFPPKPKDTL |
| | | MISRTPEVTCVVVDVSHEDPEVKFNWYVDGVEVHNAKTKPR |
| | | EEQYASTYRVVSVLTVLHQDWLNGKEYKCKVSNKALPAPIE |
| | | KTISKAKGQPREPQVCTLPPSREEMTKNQVSLSCAVKGFYPS |
| | | DIAVEWESNGQPENNYKTTPPVLDSDGSFFLVSKLTVDKSRW |
| | | QQGNVFSCSVMHEALHNHYTQKSLSLSPGK |
| SEQ ID NO: 3369 | Light chain (VL-CL) | NVVMTQTPLSLPVSLGDQASISCRSSQSLVHSNGNTYLHWYL |
| | | QKPGQSPKFLIYKVSNRFSGVPDRFSGGGSGTEFTLKISRVEA |
| | | EDLGVYFCSQSTHVPFTFGSGTKLEIKRTVAAPSVFIFPPSDEQ |
| | | LKSGTASVVCLLNNFYPREAKVQWKVDNALQSGNSQESVTE |
| | | QDSKDSTYSLSSTLTLSKADYEKHKVYACEVTHQGLSSPVTK |
| | | SFNRGEC |
| SEQ ID NO: 3340 | IL-2 C125A-Heavy | APTSSSTKKTQLQLEHLLLDLQMILNGINNYKNPKLTRMLTF |
| | Chain Constant | KFYMPKKATELKHLQCLEEELKPLEEVLNLAQSKNFHLRPRD |
| | Domain (Fc Region) | LISNINVIVLELKGSETTFMCEYADETATIVEFLNRWITFAQSII |
| | | STLTGGGGSGGGGSDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLIVLHQDWLNGKEYKCKVSNKALPAP |
| | | IEKTISKAKGQPREPQVYTLPPCREEMTKNQVSLWCLVKGFY |
| | | PSDIAVEWESNGQPENNYKTTPPVLDSDGSFFLYSKLTVDKS |
| | | RWQQGNVFSCSVMHEALHNHYTQKSLSLSPGK |
| SEQ ID NO: 3371 | HC CDR1 | SITSGYFWN |
| SEQ ID NO: 3372 | HC CDR2 | YISYDGSNNYNPSLKN |
| SEQ ID NO: 3373 | HC CDR3 | PSPGTGYAVDY |
| SEQ ID NO: 3374 | LC CDR1 | RSSQSLVHSNGNTYLH |
| SEQ ID NO: 3375 | LC CDR2 | KVSNRFS |
| SEQ ID NO: 3376 | LC CDR3 | SQSTHVPFT |
| SEQ ID NO: 3455 | VH | NVQLQESGPGLVKPSQSLSLTCSVAGYSITSGYFWNWIRQFPG |
| | | NKLEWMGYISYDGSNNYNPSLKNRISITRDTSKNQFFLKLNSV |
| | | TTEDTATYYCASPSPGTGYAVDYWGQGTSVTVSS |
| SEQ ID NO: 3456 | VL | NVVMTQTPLSLPVSLGDQASISCRSSQSLVHSNGNTYLHWYLQ |
| | | KPGQSPKFLIYKVSNRFSGVPDRFSGGGSGTEFTLKISRVEAED |
| | | LGVYFCSQSTHVPFTFGSGTKLEIK |
| SEQ ID NO: 3649 | Heavy chain constant | ASTKGPSVFPLAPSSKSTSGGTAALGCLVKDYFPEPVTVSWNS |
| | region (CH1-CH2-CH3) | GALTSGVHTFPAVLQSSGLYSLSSVVTVPSSSLGTQTYICNVNH |
| | Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSPGK |
| SEQ ID NO: 3447 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSPGK |
| SEQ ID NO: 3644 | Light chain constant | RTVAAPSVFIFPPSDEQLKSGTASVVCLLNNFYPREAKVQWKV |
| | region | DNALQSGNSQESVTEQDSKDSTYSLSSTLTLSKADYEKHKVY |
| | | ACEVTHQGLSSPVTKSFNRGEC |
| SEQ ID NO: 2270 | IL-2 C125A | APTSSSTKKTQLQLEHLLLDLQMILNGINNYKNPKLTRMLTFK |
| | | FYMPKKATELKHLQCLEEELKPLEEVLNLAQSKNFHLRPRDLI |
| | | SNINVIVLELKGSETTFMCEYADETATIVEFLNRWITFAQSIIST |
| | | LT |
| SEQ ID NO: 3308 | Linker | GGGGSGGGGS |
| SEQ ID NO: 3648 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSPGK |

TABLE 19
Exemplary agents that comprise a first binder that binds to a first target molecule,
wherein the exemplary first binder comprises anti-TCRβ V17 antibodies. Exemplary first
target molecule comprises TCRVβ 17. Exemplary anti-TCRβ V17 antibodies include, but are
not limited to, BMM0034, which binds to murine TCRβV 17. CDRs are underlined.
BMM0034, also referred to as Bl432 or BL431
Binds to murine TCRVβ 17

| | | |
|---|---|---|
| SEQ ID NO: 3378 | Heavy chain (VH-CH) | QVQLQQPGAELVRPGASVKLSCKASGYTFTNYWMNWVKQR |
| | | PGQGLEWIGMIHPSDSATRLNQKFKDKATLTVDKSSSTAYMQ |
| | | LSSPTSEDSAVYYCARWAYYGNLYAIDYWGQGTSVTVSSAK |
| | | TTAPSVYPLAPVCGDTTGSSVTLGCLVKGYFPEPVTLTWNSG |
| | | SLSSGVHTFPAVLQSDLYTLSSSVTVTSSTWPSQSITCNVAHP |
| | | ASSTKVDKKIEPRGPTIKPCPPCKCPAPNAAGGPSVFIFPPKIK |
| | | DVLMISLSPIVTCVVVDVSEDDPDVQISWFVNNVEVHTAQTQ |
| | | THREDYNSTLRVVSALPIQHQDWMSGKEFKCKVNNKDLGAP |
| | | IERTISKPKGSVRAPQVCVLPPPEEEMTKKQVTLSCAVTDFMP |
| | | EDIYVEWTNNGKTELNYKNTEPVLDSDGSYFMVSKLRVEKK |
| | | NWVERNSYSCSVVHEGLHNHHTTKSFSRTPGK |
| SEQ ID NO: 3379 | Light chain (VL-CL) | DIVLTQSPASLAVSLGQRATISCKASQSVDYDGYSYMHWFQQ |
| | | KPGQPPKLLIYAASNLESGIPARFIGSGSGTDFTLNIHPVEEED |
| | | AATYYCQQSNEDPWTFGGGTKLEIKRADAAPTVSIFPPSSEQL |
| | | TSGGASVVCFLNNFYPKDINVKWKIDGSERQNGVLNSWTDQ |
| | | DSKDSTYSMSSTLTLTKDEYERHNSYTCEATHKTSTSPIVKSF |
| | | NRNEC |
| SEQ ID NO: 3380 | IL-2-Heavy Chain | APTSSSTKKTQLQLEHLLLDLQMILNGINNYKNPKLTRMLTF |
| | Constant Domain (Fc | KFYMPKKATELKHLQCLEEELKPLEEVLNLAQSKNFHLRPRD |
| | Region) | LISNINVIVLELKGSETTFMCEYADETATIVEFLNRWITFCQSII |
| | | STLTGGGGSGGGGSTIKPCPPCKCPAPNAAGGPSVFIFPPKIKD |
| | | VLMISLSPIVTCVVVDVSEDDPDVQISWFVNNVEVHTAQTQT |
| | | HREDYNSTLRVVSALPIQHQDWMSGKEFKCKVNNKDLGAPI |
| | | ERTISKPKGSVRAPQVYVLPPCEEEMTKKQVTLWCMVTDFM |
| | | PEDIYVEWTNNGKTELNYKNTEPVLDSDGSYFMYSKLRVEK |
| | | KNWVERNSYSCSVVHEGLHNHHTTKSFSRTPGK |
| SEQ ID NO: 3381 | HC CDR1 | GYTFTNYWMN |
| SEQ ID NO: 3382 | HC CDR2 | MIHPSDSATRLNQKFKD |
| SEQ ID NO: 3383 | HC CDR3 | WAYYGNLYAIDY |
| SEQ ID NO: 3384 | LC CDR1 | KASQSVDYDGYSYMH |
| SEQ ID NO: 3325 | LC CDR2 | AASNLES |
| SEQ ID NO: 3386 | LC CDR3 | QQSNEDPWT |
| SEQ ID NO: 3457 | VH | QVQLQQPGAELVRPGASVKLSCKASGYTFTNYWMNWVKQRP |
| | | GQGLEWIGMIHPSDSATRLNQKFKDKATLTVDKSSSTAYMQL |
| | | SSPTSEDSAVYYCARWAYYGNLYAIDYWGQGTSVTVSS |
| SEQ ID NO: 3458 | VL | DIVLTQSPASLAVSLGQRATISCKASQSVDYDGYSYMHWFQQ |
| | | KPGQPPKLLIYAASNLESGIPARFIGSGSGTDFTLNIHPVEEEDA |
| | | ATYYCQQSNEDPWTFGGGTKLEIK |
| SEQ ID NO: 3459 | Heavy chain constant | AKTTAPSVYPLAPVCGDTTGSSVTLGCLVKGYFPEPVTLTWNS |
| | region (CH1-CH2-CH3) | GSLSSGVHTFPAVLQSDLYTLSSSVTVTSSTWPSQSITCNVAHP |
| | Hole | ASSTKVDKKIEPRGPTIKPCPPCKCPAPNAAGGPSVFIFPPKIKD |
| | | VLMISLSPIVTCVVVDVSEDDPDVQISWFVNNVEVHTAQTQTH |
| | | REDYNSTLRVVSALPIQHQDWMSGKEFKCKVNNKDLGAPIER |
| | | TISKPKGSVRAPQVCVLPPPEEEMTKKQVTLSCAVTDFMPEDI |
| | | YVEWTNNGKTELNYKNTEPVLDSDGSYFMVSKLRVEKKNWV |
| | | ERNSYSCSVVHEGLHNHHTTKSFSRTPGK |
| SEQ ID NO: 3460 | Heavy chain hinge- | TIKPCPPCKCPAPNAAGGPSVFIFPPKIKDVLMISLSPIVTCVVV |
| | Fc region (CH2-CH3) | DVSEDDPDVQISWFVNNVEVHTAQTQTHREDYNSTLRVVSAL |
| | Hole | PIQHQDWMSGKEFKCKVNNKDLGAPIERTISKPKGSVRAPQVC |
| | | VLPPPEEEMTKKQVTLSCAVTDFMPEDIYVEWTNNGKTELNY |
| | | KNTEPVLDSDGSYFMVSKLRVEKKNWVERNSYSCSVVHEGL |
| | | HNHHTTKSFSRTPGK |
| SEQ ID NO: 3461 | Light chain constant | RADAAPTVSIFPPSSEQLTSGGASVVCFLNNFYPKDINVKWKID |
| | region | GSERQNGVLNSWTDQDSKDSTYSMSSTLTLTKDEYERHNSYT |
| | | CEATHKTSTSPIVKSFNRNEC |
| SEQ ID NO: 2191 | IL-2 | APTSSSTKKTQLQLEHLLLDLQMILNGINNYKNPKLTRMLTFK |
| | | FYMPKKATELKHLQCLEEELKPLEEVLNLAQSKNFHLRPRDLI |
| | | SNINVIVLELKGSETTFMCEYADETATIVEFLNRWITFCQSIISTL |
| | | T |
| SEQ ID NO: 3308 | Linker | GGGGSGGGGS |
| SEQ ID NO: 3462 | Heavy chain hinge- | TIKPCPPCKCPAPNAAGGPSVFIFPPKIKDVLMISLSPIVTCVVV |
| | Fc region (CH2-CH3) | DVSEDDPDVQISWFVNNVEVHTAQTQTHREDYNSTLRVVSAL |
| | knob | PIQHQDWMSGKEFKCKVNNKDLGAPIERTISKPKGSVRAPQV |
| | | YVLPPCEEEMTKKQVTLWCMVTDFMPEDIYVEWTNNGKTEL |
| | | NYKNTEPVLDSDGSYFMYSKLRVEKKNWVERNSYSCSVVHE |
| | | GLHNHHTTKSFSRTPGK |

TABLE 20
Exemplary agents that comprise a first binder that binds to a first target molecule,
wherein the exemplary first binder comprises anti-TCR V29 antibodies. Exemplary first
target molecule comprises TCRVβ 29. Exemplary anti-TCRβ V29 antibodies include, but are
not limited to, BMM0019, which binds to murine TCRβV 29. CDRs are underlined.
BMM0019, also referred to as BL404 or BL405
Binds to murine TCRVβ 29

| | | |
|---|---|---|
| SEQ ID NO: 3388 | Heavy chain (VH-CH) | EVQLVESGGGLVQPGASLKLSCVASGFTFSDYWMSWVRQTP |
| | | GKTMEWIGNIKYDGSHTNYAPSLKNRFTISRDNAKSTLFLQM |
| | | SNVRSEDTATYYCTRESHYGYIYPFDYWGQGVMVTVSSAKT |
| | | TAPSVYPLAPVCGDTTGSSVTLGCLVKGYFPEPVTLTWNSGS |
| | | LSSGVHTFPAVLQSDLYTLSSSVTVTSSTWPSQSITCNVAHPA |
| | | SSTKVDKKIEPRGPTIKPCPPCKCPAPNAAGGPSVFIFPPKIKD |
| | | VLMISLSPIVTCVVVDVSEDDPDVQISWFVNNVEVHTAQTQT |
| | | HREDYNSTLRVVSALPIQHQDWMSGKEFKCKVNNKDLGAPI |
| | | ERTISKPKGSVRAPQVCVLPPPEEEMTKKQVTLSCAVTDFMP |
| | | EDIYVEWTNNGKTELNYKNTEPVLDSDGSYFMVSKLRVEKK |
| | | NWVERNSYSCSVVHEGLHNHHTTKSFSRTPGK |
| SEQ ID NO: 3389 | Light chain (VL-CL) | DIQMTQSPSSMSVSLGDTVTITCRASQDVGIYVNWFQQKPGK |
| | | SPRRMIYRATNLADGVPSRFSGSRSGSDYSLTISSLESEDVAD |
| | | YHCLQYDESPFTFGSGTKLEIKRADAAPTVSIFPPSSEQLTSGG |
| | | ASVVCFLNNFYPKDINVKWKIDGSERQNGVLNSWTDQDSKD |
| | | STYSMSSTLTLTKDEYERHNSYTCEATHKTSTSPIVKSFNRNE |
| | | C |
| SEQ ID NO: 3390 | IL-2-Heavy Chain | APTSSSTKKTQLQLEHLLLDLQMILNGINNYKNPKLTRMLTF |
| | Constant Domain (Fc | KFYMPKKATELKHLQCLEEELKPLEEVLNLAQSKNFHLRPRD |
| | Region) | LISNINVIVLELKGSETTFMCEYADETATIVEFLNRWITFCQSII |
| | | STLTGGGGSGGGGSTIKPCPPCKCPAPNAAGGPSVFIFPPKIKD |
| | | VLMISLSPIVTCVVVDVSEDDPDVQISWFVNNVEVHTAQTQT |
| | | HREDYNSTLRVVSALPIQHQDWMSGKEFKCKVNNKDLGAPI |
| | | ERTISKPKGSVRAPQVYVLPPCEEEMTKKQVTLWCMVTDFM |
| | | PEDIYVEWTNNGKTELNYKNTEPVLDSDGSYFMYSKLRVEK |
| | | KNWVERNSYSCSVVHEGLHNHHTTKSFSRTPGK |
| SEQ ID NO: 3391 | HC CDR1 | GFTFSDYWMS |
| SEQ ID NO: 3392 | HC CDR2 | NIKYDGSHTNYAPSLKN |
| SEQ ID NO: 3393 | HC CDR3 | ESHYGYIYPFDY |
| SEQ ID NO: 3394 | LC CDR1 | RASQDVGIYVN |
| SEQ ID NO: 3395 | LC CDR2 | RATNLAD |
| SEQ ID NO: 3396 | LC CDR3 | LQYDESPFT |
| SEQ ID NO: 3463 | VH | EVQLVESGGGLVQPGASLKLSCVASGFTFSDYWMSWVRQTPG |
| | | KTMEWIGNIKYDGSHTNYAPSLKNRFTISRDNAKSTLFLQMSN |
| | | VRSEDTATYYCTRESHYGYIYPFDYWGQGVMVTVSS |
| SEQ ID NO: 3464 | VL | DIQMTQSPSSMSVSLGDTVTITCRASQDVGIYVNWFQQKPGKS |
| | | PRRMIYRATNLADGVPSRFSGSRSGSDYSLTISSLESEDVADYH |
| | | CLQYDESPFTFGSGTKLEIK |
| SEQ ID NO: 3459 | Heavy chain constant | AKTTAPSVYPLAPVCGDTTGSSVTLGCLVKGYFPEPVTLTWNS |
| | region (CH1-CH2-CH3) | GSLSSGVHTFPAVLQSDLYTLSSSVTVTSSTWPSQSITCNVAHP |
| | Hole | ASSTKVDKKIEPRGPTIKPCPPCKCPAPNAAGGPSVFIFPPKIKD |
| | | VLMISLSPIVTCVVVDVSEDDPDVQISWFVNNVEVHTAQTQTH |
| | | REDYNSTLRVVSALPIQHQDWMSGKEFKCKVNNKDLGAPIER |
| | | TISKPKGSVRAPQVCVLPPPEEEMTKKQVTLSCAVTDFMPEDI |
| | | YVEWTNNGKTELNYKNTEPVLDSDGSYFMVSKLRVEKKNWV |
| | | ERNSYSCSVVHEGLHNHHTTKSFSRTPGK |
| SEQ ID NO: 3460 | Heavy chain hinge- | TIKPCPPCKCPAPNAAGGPSVFIFPPKIKDVLMISLSPIVTCVVV |
| | Fc region (CH2-CH3) | DVSEDDPDVQISWFVNNVEVHTAQTQTHREDYNSTLRVVSAL |
| | Hole | PIQHQDWMSGKEFKCKVNNKDLGAPIERTISKPKGSVRAPQVC |
| | | VLPPPEEEMTKKQVTLSCAVTDFMPEDIYVEWTNNGKTELNY |
| | | KNTEPVLDSDGSYFMVSKLRVEKKNWVERNSYSCSVVHEGL |
| | | HNHHTTKSFSRTPGK |
| SEQ ID NO: 3461 | Light chain constant | RADAAPTVSIFPPSSEQLTSGGASVVCFLNNFYPKDINVKWKID |
| | region | GSERQNGVLNSWTDQDSKDSTYSMSSTLTLTKDEYERHNSYT |
| | | CEATHKTSTSPIVKSFNRNEC |
| SEQ ID NO: 2191 | IL-2 | APTSSSTKKTQLQLEHLLLDLQMILNGINNYKNPKLTRMLTFK |
| | | FYMPKKATELKHLQCLEEELKPLEEVLNLAQSKNFHLRPRDLI |
| | | SNINVIVLELKGSETTFMCEYADETATIVEFLNRWITFCQSIISTL |
| | | T |
| SEQ ID NO: 3308 | Linker | GGGGSGGGGS |
| SEQ ID NO: 3462 | Heavy chain hinge- | TIKPCPPCKCPAPNAAGGPSVFIFPPKIKDVLMISLSPIVTCVVV |
| | Fc region (CH2-CH3) | DVSEDDPDVQISWFVNNVEVHTAQTQTHREDYNSTLRVVSAL |
| | knob | PIQHQDWMSGKEFKCKVNNKDLGAPIERTISKPKGSVRAPQV |
| | | YVLPPCEEEMTKKQVTLWCMVTDFMPEDIYVEWTNNGKTEL |
| | | NYKNTEPVLDSDGSYFMYSKLRVEKKNWVERNSYSCSVVHE |
| | | GLHNHHTTKSFSRTPGK |

TABLE 21
Exemplary agents that comprise a first binder that binds to a first target molecule, wherein
the exemplary first binder comprises anti-TCRβ V13 antibodies. Exemplary first target
molecule comprises TCRVB 13 (e.g., TCRVB 13-3). Exemplary anti-TCRβ V13 antibodies include,
but are not limited to, BKM0307, which binds to murine TCRβV 13-3. CDRs are underlined.
BKM0307, also referred to as BK057 or BJ1039
Binds to murine TCRVβ 13-3

| | | |
|---|---|---|
| SEQ ID NO: 3398 | Heavy chain (VH-CH) | QVQLQQSGTELMKPGASVKISCKASGYTFSNYWIEWIKQRPG |
| | | HGLEWVGEILPGAGPTNYNEKFKGKATFTADSSSNTAYMQL |
| | | SSLTSEDSAVYYCARTDYDYDWFAYWGQGTLVTVSAAKTT |
| | | APSVYPLAPVSGDTTGSSVTLGCLVKGYFPEPVTLTWNSGSL |
| | | SSGVHTFPAVLQSDLYTLSSSVTVTSSTWPSQSITCNVAHPAS |
| | | STKVDKKIEPKSCDKTHTCPPCPAPNAAGGPSVFIFPPKIKDVL |
| | | MISLSPIVTCVVVDVSEDDPDVQISWFVNNVEVHTAQTQTHR |
| | | EDYNSTLRVVSALPIQHQDWMSGKEFKCKVNNKDLGAPIER |
| | | TISKPKGSVRAPQVCVLPPPEEEMTKKQVTLSCAVTDFMPEDI |
| | | YVEWTNNGKTELNYKNTEPVLDSDGSYFMVSKLRVEKKNW |
| | | VERNSYSCSVVHEGLHNHHTTKSFSRTPGKEPEA |
| SEQ ID NO: 3399 | Light chain (VL-CL) | DIVMSQSPSSLAVSVGEKVTMSCKSSQSLLYSGNQKNYLAW |
| | | YQQKPGQSPKLLIYWASTRESGVPDRFTGSGSGTDFTLTINSV |
| | | KAEDLTVYYCQQYYGYPRTFGGGTKVEIKRADAAPTVSIFPP |
| | | SSEQLTSGGASVVCFLNNFYPKDINVKWKIDGSERQNGVLNS |
| | | WTDQDSKDSTYSMSSTLTLTKDEYERHNSYTCEATHKTSTSP |
| | | IVKSFNRNEC |
| SEQ ID NO: 3400 | IL-2-Heavy Chain | APTSSSTKKTQLQLEHLLLDLQMILNGINNYKNPKLTRMLTF |
| | Constant Domain (Fc | KFYMPKKATELKHLQCLEEELKPLEEVLNLAQSKNFHLRPRD |
| | Region) | LISNINVIVLELKGSETTFMCEYADETATIVEFLNRWITFCQSII |
| | | STLTGGGGSGGGGSDKTHTCPPCPAPNAAGGPSVFIFPPKIKD |
| | | VLMISLSPIVTCVVVDVSEDDPDVQISWFVNNVEVHTAQTQT |
| | | HREDYNSTLRVVSALPIQHQDWMSGKEFKCKVNNKDLGAPI |
| | | ERTISKPKGSVRAPQVYVLPPCEEEMTKKQVTLWCMVTDFM |
| | | PEDIYVEWTNNGKTELNYKNTEPVLDSDGSYFMYSKLRVEK |
| | | KNWVERNSYSCSVVHEGLHNHHTTKSFSRTPGKGGGGSHHH |
| | | HHHHH |
| SEQ ID NO: 3401 | HC CDR1 | GYTFSNYWIE |
| SEQ ID NO: 3402 | HC CDR2 | EILPGAGPTNYNEKFKG |
| SEQ ID NO: 3403 | HC CDR3 | TDYDYDWFAY |
| SEQ ID NO: 3404 | LC CDR1 | KSSQSLLYSGNQKNYLA |
| SEQ ID NO: 3405 | LC CDR2 | WASTRES |
| SEQ ID NO: 3406 | LC CDR3 | QQYYGYPRT |
| SEQ ID NO: 3465 | VH | QVQLQQSGTELMKPGASVKISCKASGYTFSNYWIEWIKQRPG |
| | | HGLEWVGEILPGAGPTNYNEKFKGKATFTADSSSNTAYMQLS |
| | | SLTSEDSAVYYCARTDYDYDWFAYWGQGTLVTVSA |
| SEQ ID NO: 3466 | VL | DIVMSQSPSSLAVSVGEKVTMSCKSSQSLLYSGNQKNYLAWY |
| | | QQKPGQSPKLLIYWASTRESGVPDRFTGSGSGTDFTLTINSVKA |
| | | EDLTVYYCQQYYGYPRTFGGGTKVEIK |
| SEQ ID NO: 3467 | Heavy chain constant | AKTTAPSVYPLAPVSGDTTGSSVTLGCLVKGYFPEPVTLTWNS |
| | region (CH1-CH2-CH3) | GSLSSGVHTFPAVLQSDLYTLSSSVTVTSSTWPSQSITCNVAHP |
| | Hole | ASSTKVDKKIEPKSCDKTHTCPPCPAPNAAGGPSVFIFPPKIKD |
| | | VLMISLSPIVTCVVVDVSEDDPDVQISWFVNNVEVHTAQTQTH |
| | | REDYNSTLRVVSALPIQHQDWMSGKEFKCKVNNKDLGAPIER |
| | | TISKPKGSVRAPQVCVLPPPEEEMTKKQVTLSCAVTDFMPEDI |
| | | YVEWTNNGKTELNYKNTEPVLDSDGSYFMVSKLRVEKKNWV |
| | | ERNSYSCSVVHEGLHNHHTTKSFSRTPGKEPEA |
| SEQ ID NO: 3468 | Heavy chain hinge- | DKTHTCPPCPAPNAAGGPSVFIFPPKIKDVLMISLSPIVTCVVVD |
| | Fc region (CH2-CH3) | VSEDDPDVQISWFVNNVEVHTAQTQTHREDYNSTLRVVSALPI |
| | Hole | QHQDWMSGKEFKCKVNNKDLGAPIERTISKPKGSVRAPQVCV |
| | | LPPPEEEMTKKQVTLSCAVTDFMPEDIYVEWTNNGKTELNYK |
| | | NTEPVLDSDGSYFMVSKLRVEKKNWVERNSYSCSVVHEGLH |
| | | NHHTTKSFSRTPGKEPEA |
| SEQ ID NO: 3461 | Light chain constant | RADAAPTVSIFPPSSEQLTSGGASVVCFLNNFYPKDINVKWKID |
| | region | GSERQNGVLNSWTDQDSKDSTYSMSSTLTLTKDEYERHNSYT |
| | | CEATHKTSTSPIVKSFNRNEC |
| SEQ ID NO: 2191 | IL-2 | APTSSSTKKTQLQLEHLLLDLQMILNGINNYKNPKLTRMLTFK |
| | | FYMPKKATELKHLQCLEEELKPLEEVLNLAQSKNFHLRPRDLI |
| | | SNINVIVLELKGSETTFMCEYADETATIVEFLNRWITFCQSIISTL |
| | | T |
| SEQ ID NO: 3308 | Linker | GGGGSGGGGS |
| SEQ ID NO: 3469 | Heavy chain hinge- | DKTHTCPPCPAPNAAGGPSVFIFPPKIKDVLMISLSPIVTCVVVD |
| | Fc region (CH2-CH3) | VSEDDPDVQISWFVNNVEVHTAQTQTHREDYNSTLRVVSALPI |
| | knob | QHQDWMSGKEFKCKVNNKDLGAPIERTISKPKGSVRAPQVYV |
| | | LPPCEEEMTKKQVTLWCMVTDFMPEDIYVEWTNNGKTELNY |
| | | KNTEPVLDSDGSYFMYSKLRVEKKNWVERNSYSCSVVHEGL |
| | | HNHHTTKSFSRTPGKGGGGSHHHHHHHH |

TABLE 22
Exemplary agents that comprise a first binder that binds to a first target molecule,
wherein the exemplary first binder comprises anti-TCRα V19 antibodies. Exemplary first
target molecule comprises TCRVA 19. Exemplary anti-TCRα V19 antibodies include, but are
not limited to, BMM0305, which binds to human TCRα V19. CDRs are underlined.
BMM0305, also referred to as BM153 or BM154
Binds to human TCRVα 19

| | | |
|---|---|---|
| SEQ ID NO: 3408 | Heavy chain (VH-CH) | QIQLVQSGPELKKPGETVKISCKASGYTFTDYSMHWMKQAP |
| | | GKGLKWMAWINTDTGEPTYADDFKGRFAFSLETSVSTAYLQI |
| | | NNLKNEDTATYFCARSSRRFDYWGQGTTLTVSSASTKGPSVF |
| | | PLAPSSKSTSGGTAALGCLVKDYFPEPVTVSWNSGALTSGVH |
| | | TFPAVLQSSGLYSLSSVVTVPSSSLGTQTYICNVNHKPSNTKV |
| | | DKRVEPKSCDKTHTCPPCPAPELLGGPSVFLFPPKPKDTLMIS |
| | | RTPEVTCVVVDVSHEDPEVKFNWYVDGVEVHNAKTKPREEQ |
| | | YASTYRVVSVLTVLHQDWLNGKEYKCKVSNKALPAPIEKTIS |
| | | KAKGQPREPQVCTLPPSREEMTKNQVSLSCAVKGFYPSDIAV |
| | | EWESNGQPENNYKTTPPVLDSDGSFFLVSKLTVDKSRWQQG |
| | | NVFSCSVMHEALHNHYTQKSLSLSPGK |
| SEQ ID NO: 3409 | Light chain (VL-CL) | NIVMTQSPKSMSMSVGERVTLSCKASENVGTYVSWYQQKPE |
| | | QSPKLLIYGASNRYTGVPDRFTGSGSAKDFTLTIRSVQAEDLA |
| | | DYHCGQSYISPYTFGGGTKLEIKRTVAAPSVFIFPPSDEQLKSG |
| | | TASVVCLLNNFYPREAKVQWKVDNALQSGNSQESVTEQDSK |
| | | DSTYSLSSTLTLSKADYEKHKVYACEVTHQGLSSPVTKSFNR |
| | | GEC |
| SEQ ID NO: 3340 | IL-2 C125A-Heavy | APTSSSTKKTQLQLEHLLLDLQMILNGINNYKNPKLTRMLTF |
| | Chain Constant | KFYMPKKATELKHLQCLEEELKPLEEVLNLAQSKNFHLRPRD |
| | Domain (Fc Region) | LISNINVIVLELKGSETTFMCEYADETATIVEFLNRWITFAQSII |
| | | STLTGGGGSGGGGSDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVYTLPPCREEMTKNQVSLWCLVKGFY |
| | | PSDIAVEWESNGQPENNYKTTPPVLDSDGSFFLYSKLTVDKS |
| | | RWQQGNVFSCSVMHEALHNHYTQKSLSLSPGK |
| SEQ ID NO: 3411 | HC CDR1 | GYTFTDYSMH |
| SEQ ID NO: 3412 | HC CDR2 | WINTDTGEPTYADDFKG |
| SEQ ID NO: 3413 | HC CDR3 | SSRRFDY |
| SEQ ID NO: 3414 | LC CDR1 | KASENVGTYVS |
| SEQ ID NO: 3415 | LC CDR2 | GASNRYT |
| SEQ ID NO: 3416 | LC CDR3 | GQSYISPYT |
| SEQ ID NO: 3470 | VH | QIQLVQSGPELKKPGETVKISCKASGYTFTDYSMHWMKQAPG |
| | | KGLKWMAWINTDTGEPTYADDFKGRFAFSLETSVSTAYLQIN |
| | | NLKNEDTATYFCARSSRRFDYWGQGTTLTVSS |
| SEQ ID NO: 3471 | VL | NIVMTQSPKSMSMSVGERVTLSCKASENVGTYVSWYQQKPEQ |
| | | SPKLLIYGASNRYTGVPDRFTGSGSAKDFTLTIRSVQAEDLAD |
| | | YHCGQSYISPYTFGGGTKLEIK |
| SEQ ID NO: 3649 | Heavy chain constant | ASTKGPSVFPLAPSSKSTSGGTAALGCLVKDYFPEPVTVSWNS |
| | region (CH1-CH2-CH3) | GALTSGVHTFPAVLQSSGLYSLSSVVTVPSSSLGTQTYICNVNH |
| | Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSPGK |
| SEQ ID NO: 3447 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSPGK |
| SEQ ID NO: 3644 | Light chain constant | RTVAAPSVFIFPPSDEQLKSGTASVVCLLNNFYPREAKVQWKV |
| | region | DNALQSGNSQESVTEQDSKDSTYSLSSTLILSKADYEKHKVY |
| | | ACEVTHQGLSSPVTKSFNRGEC |
| SEQ ID NO: 2270 | IL-2 C125A | APTSSSTKKTQLQLEHLLLDLQMILNGINNYKNPKLTRMLTFK |
| | | FYMPKKATELKHLQCLEEELKPLEEVLNLAQSKNFHLRPRDLI |
| | | SNINVIVLELKGSETTFMCEYADETATIVEFLNRWITFAQSIIST |
| | | LT |
| SEQ ID NO: 3308 | Linker | GGGGSGGGGS |
| SEQ ID NO: 3648 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSPGK |

TABLE 23
Exemplary agents that comprise a first binder that binds to a first target molecule,
wherein the exemplary first binder comprises anti-TCRVβ 6 antibodies. Exemplary first
target molecule comprises TCRVβ 6 (e.g., TCRVβ 6-5). Exemplary anti-TCRVβ 6 antibodies
include, but are not limited to, BMM0306, BMM0354, BMM0356, which binds to human TCRVβ 6
(e.g., TCRVβ 6-5). CDRs are underlined.
BMM0306

| | | |
|---|---|---|
| SEQ ID NO: 3348 | Heavy chain (VH-CH)- | QVQLVQSGAEVKKPGSSVKVSCKASGHDFRLTYIHWVRQAP |
| | Hole | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHKP |
| | | SNTKVDKRVEPKSCDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVCTLPPSREEMTKNQVSLSCAVKGFYP |
| | | SDIAVEWESNGQPENNYKTTPPVLDSDGSFFLVSKLTVDKSR |
| | | WQQGNVFSCSVMHEALHNHYTQKSLSLSP |
| SEQ ID NO: 3502 | Heavy chain (VH-CH)- | QVQLVQSGAEVKKPGSSVKVSCKASGHDFRLTYIHWVRQAP |
| | Hole | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHKP |
| | | SNTKVDKRVEPKSCDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLIVLHQDWLNGKEYKCKVSNKALPAP |
| | | IEKTISKAKGQPREPQVCTLPPSREEMTKNQVSLSCAVKGFYP |
| | | SDIAVEWESNGQPENNYKTTPPVLDSDGSFFLVSKLTVDKSR |
| | | WQQGNVFSCSVMHEALHNHYTQKSLSLSPGK |
| SEQ ID NO: 3349 | Light chain (VL-CL) | DIQMTQSPSFLSASVGDRVTITCKASQNVADRVVWHQQKPG |
| | | KAPKALIYSSSHRYKGVPSRFSGSGSGTEFTLTISSLQPEDFAT |
| | | YFCQQFKSYPLTFGQGTKLEIKRTVAAPSVFIFPPSDEQLKSGT |
| | | ASVVCLLNNFYPREAKVQWKVDNALQSGNSQESVTEQDSKD |
| | | STYSLSSTLTLSKADYEKHKVYACEVTHQGLSSPVTKSFNRG |
| | | EC |
| SEQ ID NO: 3420 | IL-15Rαsushi-IL-15 | ITCPPPMSVEHADIWVKSYSLYSRERYICNSGFKRKAGTSSLT |
| | Heavy Chain Constant | ECVLNKATNVAHWTTPSLKCIRDPALVHQRPAPPSGGSGGG |
| | Domain (Fc Region)- | GSGGGSGGGGSGGGNWVNVISDLKKIEDLIQSMHIDATLYTE |
| | Knob | SDVHPSCKVTAMKCFLLELQVISLESGDASIHDTVENLIILAN |
| | | NSLSSNGNVTESGCKECEELEEKNIKEFLQSFVHIVQMFINTSG |
| | | GGGSGGGGSDKTHTCPPCPAPELLGGPSVFLFPPKPKDTLMIS |
| | | RTPEVTCVVVDVSHEDPEVKFNWYVDGVEVHNAKTKPREEQ |
| | | YASTYRVVSVLTVLHQDWLNGKEYKCKVSNKALPAPIEKTIS |
| | | KAKGQPREPQVYTLPPCREEMTKNQVSLWCLVKGFYPSDIA |
| | | VEWESNGQPENNYKTTPPVLDSDGSFFLYSKLTVDKSRWQQ |
| | | GNVFSCSVMHEALHNHYTQKSLSLSPGK |
| SEQ ID NO: 3351 | HC CDR1 | GHDFRLTYIH |
| SEQ ID NO: 3421 | HC CDR1 | LTYIH |
| SEQ ID NO: 3702 | HC CDR1 | FRLTYIH |
| SEQ ID NO: 3352 | HC CDR2 | RVSAGSGNVKYNEKFKG |
| SEQ ID NO: 3353 | HC CDR3 | SYYSYDVLDY |
| SEQ ID NO: 3354 | LC CDR1 | KASQNVADRVV |
| SEQ ID NO: 3355 | LC CDR2 | SSSHRYK |
| SEQ ID NO: 3356 | LC CDR3 | QQFKSYPLT |
| SEQ ID NO: 1346 | VH | QVQLVQSGAEVKKPGSSVKVSCKASGHDERLTYIHWVRQAPG |
| | | QGLEWMGRVSAGSGNVKYNEKFKGRVTITADTSTSTAYMELS |
| | | SLRSEDTAVYYCAVSYYSYDVLDYWGQGTTVTVSS |
| SEQ ID NO: 1349 | VL | DIQMTQSPSFLSASVGDRVTITCKASQNVADRVVWHQQKPGK |
| | | APKALIYSSSHRYKGVPSRFSGSGSGTEFTLTISSLQPEDFATYF |
| | | CQQFKSYPLTFGQGTKLEIK |
| SEQ ID NO: 3452 | Heavy chain constant | ASTKGPSVFPLAPSSKSTSGGTAALGCLVKDYFPEPVTVSWNS |
| | region (CH1-CH2-CH3) | GALTSGVHTFPAVLQSSGLYSLSSVVTVPSSSLGTQTYICNVNH |
| | Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSP |
| SEQ ID NO: 3504 | Heavy chain constant | ASTKGPSVFPLAPSSKSTSGGTAALGCLVKDYFPEPVTVSWNS |
| | region (CH1-CH2-CH3) | GALTSGVHTFPAVLQSSGLYSLSSVVTVPSSSLGTQTYICNVNH |
| | Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSPGK |
| SEQ ID NO: 3453 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSP |
| SEQ ID NO: 3505 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSPGK |
| SEQ ID NO: 3644 | Light chain constant | RTVAAPSVFIFPPSDEQLKSGTASVVCLLNNFYPREAKVQWKV |
| | region | DNALQSGNSQESVTEQDSKDSTYSLSSTLTLSKADYEKHKVY |
| | | ACEVTHQGLSSPVTKSFNRGEC |
| SEQ ID NO: 3472 | IL-15Rαsushi (AA 31- | ITCPPPMSVEHADIWVKSYSLYSRERYICNSGFKRKAGTSSLTE |
| | 108 of IL-15Ralpha) | CVLNKATNVAHWTTPSLKCIRDPALVHQRPAPPS |
| SEQ ID NO: 3473 | Linker | GGSGGGGSGGGSGGGGSGGG |
| SEQ ID NO: 2170 | AA 49-162 of IL-15 | NWVNVISDLKKIEDLIQSMHIDATLYTESDVHPSCKVTAMKCF |
| | | LLELQVISLESGDASIHDTVENLIILANNSLSSNGNVTESGCKEC |
| | | EELEEKNIKEFLQSFVHIVQMFINTS |
| SEQ ID NO: 3474 | IL-15Rαsushi-IL-15 | ITCPPPMSVEHADIWVKSYSLYSRERYICNSGFKRKAGTSSLTE |
| | (AA 31-108 of IL- | CVLNKATNVAHWTTPSLKCIRDPALVHQRPAPPSGGSGGGGS |
| | 15Ralpha-Linker-AA | GGGSGGGGSGGGNWVNVISDLKKIEDLIQSMHIDATLYTESDV |
| | 31-108 of IL- | HPSCKVTAMKCFLLELQVISLESGDASIHDTVENLIILANNSLSS |
| | 15Ralpha) | NGNVTESGCKECEELEEKNIKEFLQSFVHIVQMFINTS |
| SEQ ID NO: 3308 | Linker | GGGGSGGGGS |
| SEQ ID NO: 3648 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSPGK |

BMM0354

| | | |
|---|---|---|
| SEQ ID NO: 3348 | Heavy chain (VH-CH)- | QVQLVQSGAEVKKPGSSVKVSCKASGHDFRLTYIHWVRQAP |
| | Hole | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHKP |
| | | SNTKVDKRVEPKSCDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVCTLPPSREEMTKNQVSLSCAVKGFYP |
| | | SDIAVEWESNGQPENNYKTTPPVLDSDGSFFLVSKLTVDKSR |
| | | WQQGNVFSCSVMHEALHNHYTQKSLSLSP |
| SEQ ID NO: 3502 | Heavy chain (VH-CH)- | QVQLVQSGAEVKKPGSSVKVSCKASGHDFRLTYIHWVRQAP |
| | Hole | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHKP |
| | | SNTKVDKRVEPKSCDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVCTLPPSREEMTKNQVSLSCAVKGFYP |
| | | SDIAVEWESNGQPENNYKTTPPVLDSDGSFFLVSKLTVDKSR |
| | | WQQGNVFSCSVMHEALHNHYTQKSLSLSPGK |
| SEQ ID NO: 3428 | Light chain (VL-CL) | DIQMTQSPSFLSASVGDRVTITCKASQNVADRVVWHQQKPG |
| | | KAPKALIYAASYLQSGVPSRFSGSGSGTEFTLTISSLQPEDFAT |
| | | YFCQQFKSYPLTFGQGTKLEIKRTVAAPSVFIFPPSDEQLKSGT |
| | | ASVVCLLNNFYPREAKVQWKVDNALQSGNSQESVTEQDSKD |
| | | STYSLSSTLTLSKADYEKHKVYACEVTHQGLSSPVTKSFNRG |
| | | EC |
| SEQ ID NO: 3420 | IL-15Rαsushi-IL-15 | ITCPPPMSVEHADIWVKSYSLYSRERYICNSGFKRKAGTSSLT |
| | Heavy Chain Constant | ECVLNKATNVAHWTTPSLKCIRDPALVHQRPAPPSGGSGGG |
| | Domain (Fc Region)- | GSGGGSGGGGSGGGNWVNVISDLKKIEDLIQSMHIDATLYTE |
| | Knob | SDVHPSCKVTAMKCFLLELQVISLESGDASIHDTVENLIILAN |
| | | NSLSSNGNVTESGCKECEELEEKNIKEFLQSFVHIVQMFINTSG |
| | | GGGSGGGGSDKTHTCPPCPAPELLGGPSVFLFPPKPKDTLMIS |
| | | RTPEVTCVVVDVSHEDPEVKFNWYVDGVEVHNAKTKPREEQ |
| | | YASTYRVVSVLTVLHQDWLNGKEYKCKVSNKALPAPIEKTIS |
| | | KAKGQPREPQVYTLPPCREEMTKNQVSLWCLVKGFYPSDIA |
| | | VEWESNGQPENNYKTTPPVLDSDGSFFLYSKLTVDKSRWQQ |
| | | GNVFSCSVMHEALHNHYTQKSLSLSPGK |
| SEQ ID NO: 3351 | HC CDR1 | GHDFRLTYIH |
| SEQ ID NO: 3421 | HC CDR1 | LTYIH |
| SEQ ID NO: 3702 | HC CDR1 | FRLTYIH |
| SEQ ID NO: 3352 | HC CDR2 | RVSAGSGNVKYNEKFKG |
| SEQ ID NO: 3353 | HC CDR3 | SYYSYDVLDY |
| SEQ ID NO: 3354 | LC CDR1 | KASQNVADRVV |
| SEQ ID NO: 3443 | LC CDR2 | AASYLQS |
| SEQ ID NO: 3356 | LC CDR3 | QQFKSYPLT |
| SEQ ID NO: 1346 | VH | QVQLVQSGAEVKKPGSSVKVSCKASGHDFRLTYIHWVRQAPG |
| | | QGLEWMGRVSAGSGNVKYNEKFKGRVTITADTSTSTAYMELS |
| | | SLRSEDTAVYYCAVSYYSYDVLDYWGQGTTVTVSS |
| SEQ ID NO: 3475 | VL | DIQMTQSPSFLSASVGDRVTITCKASQNVADRVVWHQQKPGK |
| | | APKALIYAASYLQSGVPSRFSGSGSGTEFTLTISSLQPEDFATYF |
| | | CQQFKSYPLTFGQGTKLEIK |
| SEQ ID NO: 3452 | Heavy chain constant | ASTKGPSVFPLAPSSKSTSGGTAALGCLVKDYFPEPVTVSWNS |
| | region (CH1-CH2-CH3) | GALTSGVHTFPAVLQSSGLYSLSSVVTVPSSSLGTQTYICNVNH |
| | Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSP |
| SEQ ID NO: 3504 | Heavy chain constant | ASTKGPSVFPLAPSSKSTSGGTAALGCLVKDYFPEPVTVSWNS |
| | region (CH1-CH2-CH3) | GALTSGVHTFPAVLQSSGLYSLSSVVTVPSSSLGTQTYICNVNH |
| | Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSPGK |
| SEQ ID NO: 3453 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSP |
| SEQ ID NO: 3505 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSPGK |
| SEQ ID NO: 3644 | Light chain constant | RTVAAPSVFIFPPSDEQLKSGTASVVCLLNNFYPREAKVQWKV |
| | region | DNALQSGNSQESVTEQDSKDSTYSLSSTLTLSKADYEKHKVY |
| | | ACEVTHQGLSSPVTKSFNRGEC |
| SEQ ID NO: 3472 | IL-15Rαsushi (AA 31- | ITCPPPMSVEHADIWVKSYSLYSRERYICNSGFKRKAGTSSLTE |
| | 108 of IL-15Ralpha) | CVLNKATNVAHWTTPSLKCIRDPALVHQRPAPPS |
| SEQ ID NO: 3473 | Linker | GGSGGGGSGGGSGGGGSGGG |
| SEQ ID NO: 2170 | AA 49-162 of IL-15 | NWVNVISDLKKIEDLIQSMHIDATLYTESDVHPSCKVTAMKCF |
| | | LLELQVISLESGDASIHDTVENLIILANNSLSSNGNVTESGCKEC |
| | | EELEEKNIKEFLQSFVHIVQMFINTS |
| SEQ ID NO: 3474 | IL-15Rαsushi-IL-15 | ITCPPPMSVEHADIWVKSYSLYSRERYICNSGFKRKAGTSSLTE |
| | (AA 31-108 of IL- | CVLNKATNVAHWTTPSLKCIRDPALVHQRPAPPSGGSGGGGS |
| | 15Ralpha-Linker-AA | GGGSGGGGSGGGNWVNVISDLKKIEDLIQSMHIDATLYTESDV |
| | 31-108 of IL- | HPSCKVTAMKCFLLELQVISLESGDASIHDTVENLIILANNSLSS |
| | 15Ralpha) | NGNVTESGCKECEELEEKNIKEFLQSFVHIVQMFINTS |
| SEQ ID NO: 3308 | Linker | GGGGSGGGGS |
| SEQ ID NO: 3648 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSPGK |

BMM0356

| | | |
|---|---|---|
| SEQ ID NO: 3348 | Heavy chain (VH-CH)- | QVQLVQSGAEVKKPGSSVKVSCKASGHDFRLTYIHWVRQAP |
| | Hole | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHKP |
| | | SNTKVDKRVEPKSCDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVCTLPPSREEMTKNQVSLSCAVKGFYP |
| | | SDIAVEWESNGQPENNYKTTPPVLDSDGSFFLVSKLTVDKSR |
| | | WQQGNVFSCSVMHEALHNHYTQKSLSLSP |
| SEQ ID NO: 3502 | Heavy chain (VH-CH)- | QVQLVQSGAEVKKPGSSVKVSCKASGHDFRLTYIHWVRQAP |
| | Hole | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHKP |
| | | SNTKVDKRVEPKSCDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVCTLPPSREEMTKNQVSLSCAVKGFYP |
| | | SDIAVEWESNGQPENNYKTTPPVLDSDGSFFLVSKLTVDKSR |
| | | WQQGNVFSCSVMHEALHNHYTQKSLSLSPGK |
| SEQ ID NO: 3437 | Light chain (VL-CL) | DIQMTQSPSFLSASVGDRVTITCKASQNVADRVVWHQQKPG |
| | | KAPKALIFAASYLQSGVPSRFSGSGSGTEFTLTISSLQPEDFAT |
| | | YFCQQFKSYPLTFGQGTKLEIKRTVAAPSVFIFPPSDEQLKSGT |
| | | ASVVCLLNNFYPREAKVQWKVDNALQSGNSQESVTEQDSKD |
| | | STYSLSSTLTLSKADYEKHKVYACEVTHQGLSSPVTKSFNRG |
| | | EC |
| SEQ ID NO: 3420 | IL-15Rαsushi-IL-15 | ITCPPPMSVEHADIWVKSYSLYSRERYICNSGFKRKAGTSSLT |
| | Heavy Chain Constant | ECVLNKATNVAHWTTPSLKCIRDPALVHQRPAPPSGGSGGG |
| | Domain (Fc Region)- | GSGGGSGGGGSGGGNWVNVISDLKKIEDLIQSMHIDATLYTE |
| | Knob | SDVHPSCKVTAMKCFLLELQVISLESGDASIHDTVENLIILAN |
| | | NSLSSNGNVTESGCKECEELEEKNIKEFLQSFVHIVQMFINTSG |
| | | GGGSGGGGSDKTHTCPPCPAPELLGGPSVFLFPPKPKDTLMIS |
| | | RTPEVTCVVVDVSHEDPEVKFNWYVDGVEVHNAKTKPREEQ |
| | | YASTYRVVSVLTVLHQDWLNGKEYKCKVSNKALPAPIEKTIS |
| | | KAKGQPREPQVYTLPPCREEMTKNQVSLWCLVKGFYPSDIA |
| | | VEWESNGQPENNYKTTPPVLDSDGSFFLYSKLTVDKSRWQQ |
| | | GNVFSCSVMHEALHNHYTQKSLSLSPGK |
| SEQ ID NO: 3351 | HC CDR1 | GHDFRLTYIH |
| SEQ ID NO: 3421 | HC CDR1 | TYIH |
| SEQ ID NO: 3702 | HC CDR1 | FRLTYIH |
| SEQ ID NO: 3352 | HC CDR2 | RVSAGSGNVKYNEKFKG |
| SEQ ID NO: 3353 | HC CDR3 | SYYSYDVLDY |
| SEQ ID NO: 3354 | LC CDR1 | KASQNVADRVV |
| SEQ ID NO: 3443 | LC CDR2 | AASYLQS |
| SEQ ID NO: 3356 | LC CDR3 | QQFKSYPLT |
| SEQ ID NO: 1346 | VH | QVQLVQSGAEVKKPGSSVKVSCKASGHDFRLTYIHWVRQAPG |
| | | QGLEWMGRVSAGSGNVKYNEKFKGRVTITADTSTSTAYMELS |
| | | SLRSEDTAVYYCAVSYYSYDVLDYWGQGTTVTVSS |
| SEQ ID NO: 3476 | VL | DIQMTQSPSFLSASVGDRVTITCKASQNVADRVVWHQQKPGK |
| | | APKALIFAASYLQSGVPSRFSGSGSGTEFTLTISSLQPEDFATYF |
| | | CQQFKSYPLTFGQGTKLEIK |
| SEQ ID NO: 3452 | Heavy chain constant | ASTKGPSVFPLAPSSKSTSGGTAALGCLVKDYFPEPVTVSWNS |
| | region (CH1-CH2-CH3) | GALTSGVHTFPAVLQSSGLYSLSSVVTVPSSSLGTQTYICNVNH |
| | Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSP |
| SEQ ID NO: 3504 | Heavy chain constant | ASTKGPSVFPLAPSSKSTSGGTAALGCLVKDYFPEPVTVSWNS |
| | region (CH1-CH2-CH3) | GALTSGVHTFPAVLQSSGLYSLSSVVTVPSSSLGTQTYICNVNH |
| | Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSPGK |
| SEQ ID NO: 3453 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSP |
| SEQ ID NO: 3505 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSPGK |
| SEQ ID NO: 3644 | Light chain constant | RTVAAPSVFIFPPSDEQLKSGTASVVCLLNNFYPREAKVQWKV |
| | region | DNALQSGNSQESVTEQDSKDSTYSLSSTLTLSKADYEKHKVY |
| | | ACEVTHQGLSSPVTKSFNRGEC |
| SEQ ID NO: 3472 | IL-15Rαsushi (AA 31- | ITCPPPMSVEHADIWVKSYSLYSRERYICNSGFKRKAGTSSLTE |
| | 108 of IL-15Ralpha) | CVLNKATNVAHWTTPSLKCIRDPALVHQRPAPPS |
| SEQ ID NO: 3473 | Linker | GGSGGGGSGGGSGGGGSGGG |
| SEQ ID NO: 2170 | AA 49-162 of IL-15 | NWVNVISDLKKIEDLIQSMHIDATLYTESDVHPSCKVTAMKCF |
| | | LLELQVISLESGDASIHDTVENLIILANNSLSSNGNVTESGCKEC |
| | | EELEEKNIKEFLQSFVHIVQMFINTS |
| SEQ ID NO: 3474 | IL-15Rαsushi-IL-15 | ITCPPPMSVEHADIWVKSYSLYSRERYICNSGFKRKAGTSSLTE |
| | (AA 31-108 of IL- | CVLNKATNVAHWTTPSLKCIRDPALVHQRPAPPSGGSGGGGS |
| | 15Ralpha-Linker-AA | GGGSGGGGSGGGNWVNVISDLKKIEDLIQSMHIDATLYTESDV |
| | 31-108 of IL- | HPSCKVTAMKCFLLELQVISLESGDASIHDTVENLIILANNSLSS |
| | 15Ralpha) | NGNVTESGCKECEELEEKNIKEFLQSFVHIVQMFINTS |
| SEQ ID NO: 3308 | Linker | GGGGSGGGGS |
| SEQ ID NO: 3648 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSPGK |

### EXAMPLES

The present disclosure will be more specifically illustrated by the following Examples. However, it should be understood that the present disclosure is not limited by these examples in any manner.

### Example 1: Reduction of Tumor Volume with Sequential Administration of Anti-Mouse TCRβV Antibodies

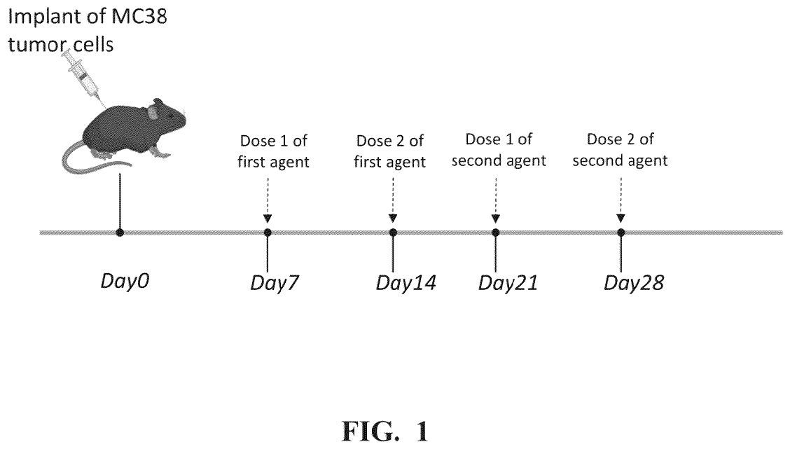

MC38 tumor cells were injected into mice. Tumor volumes were measured 7 days post injection. Mice were divided into 7 groups according to Table 24. Two doses of the first agent and two doses of the second agents were administered weekly over the course of 28 days according to the regimen in FIG. 1. In G02, G05, and G07, the first agent and second agent were the same. In G03, G04, and G06, the first agent and the second agent were different. The first anti-TCPRβV antibody was administered to tumor-bearing mice at day 7 and day 14. The second anti-TCRβV antibody was administered to tumor-bearing mice at day 21 and day 28. Tumor volume in each mouse was measured on days 7, 14, 21, and 28 days after injection of MC38 tumor cells.

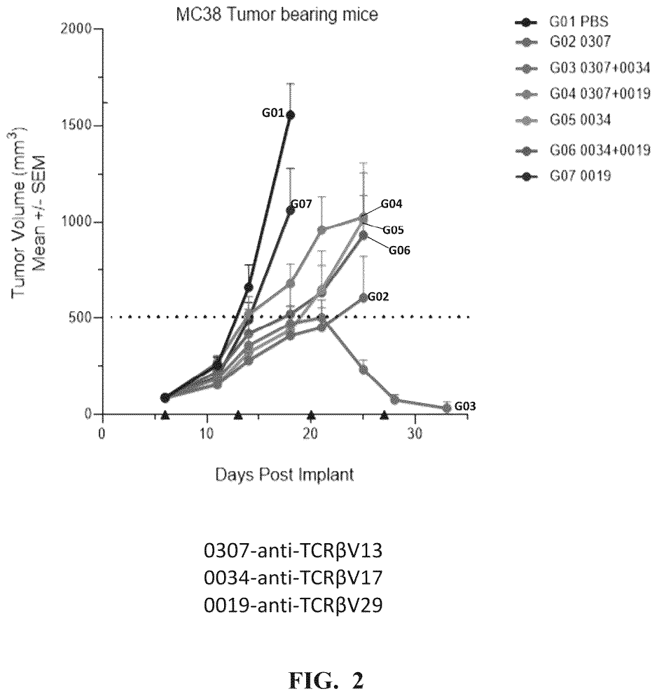

FIG. 2 depicts the exemplary results of the sequential anti-TCRβV administration. Administration of four doses 0307 in G02 significantly slowed tumor growth compared to the G01 PBS control group. Administration of four doses 0034 in G05 significantly slowed tumor growth compared to the G01 PBS control group, but not as strongly as in G02. Administration of four doses 0019 in G07 did not significantly slow tumor growth. Unexpectedly, sequential administration of two doses of 0307 followed by two doses of 0034 in G03 significantly reduced tumor volume compared to the G01 PBS control group. In contrast, sequential administration of 0307 followed by 0019 and 0034 followed by 0019 did not slow tumor growth than their respective single agent alone.

TABLE 24
Control and treatment mouse cohorts

| | First agent | Second agent |
|---|---|---|
| G01 | PBS | PBS |
| G02 | 0307 anti-mouse TCRβV13 | 0307 anti-mouse TCRβV13 |
| G03 | 0307 anti-mouse TCRβV13 | 0034 anti-mouse TCRβV17 |
| G04 | 0307 anti-mouse TCRβV13 | 0019 anti-mouse TCRβV29 |
| G05 | 0034 anti-mouse TCRβV17 | 0034 anti-mouse TCRβV17 |
| G06 | 0034 anti-mouse TCRβV17 | 0019 anti-mouse TCRβV29 |
| G07 | 0019 anti-mouse TCRβV29 | 0019 anti-mouse TCRβV29 |

### Example 2: Baseline Frequency of Various Exemplary TCRβVs (Also Referred as TCRVβs)

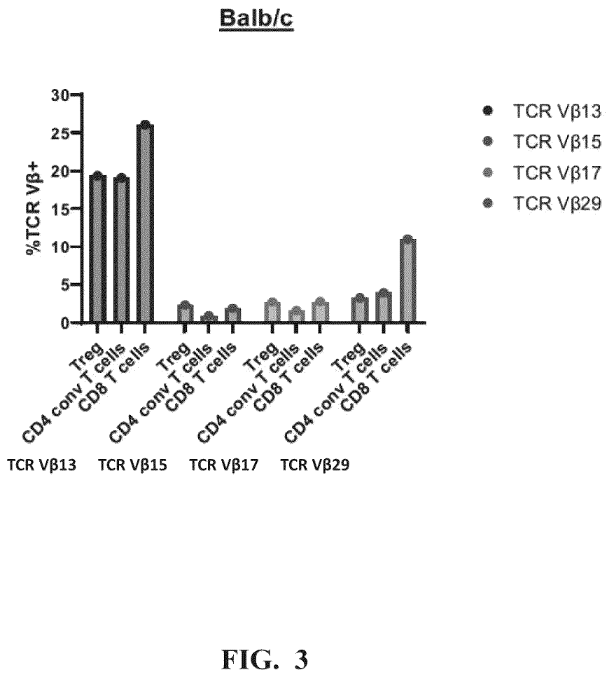

The baseline frequency of various exemplary TCRβVs, for example, mouse TCR Vβ13 (“TCR Vβ13”), mouse TCR Vβ15 (“TCR Vβ15”), mouse TCR Vβ17(“TCR Vβ17”), and mouse TCR Vβ29 (“TCR Vβ29”) in Treg cells, CD4 conventional T cells (“CD4 conv T cells), and CD8 T cells were measured in balb/c and c57bl/6 albino mice (FIG. 3).

### Example 3: Anti-Tumor Effects of the Exemplary Agents as Provided Herein, in the Mouse CT26 Tumor Model

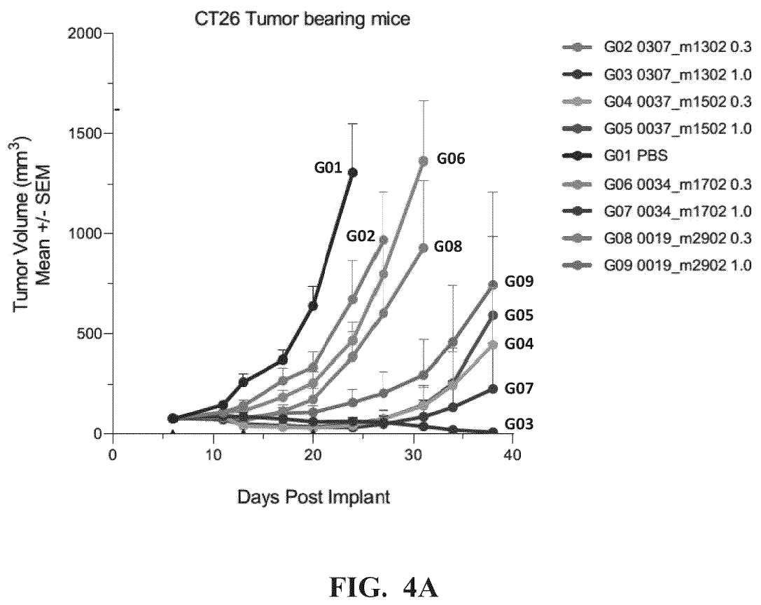

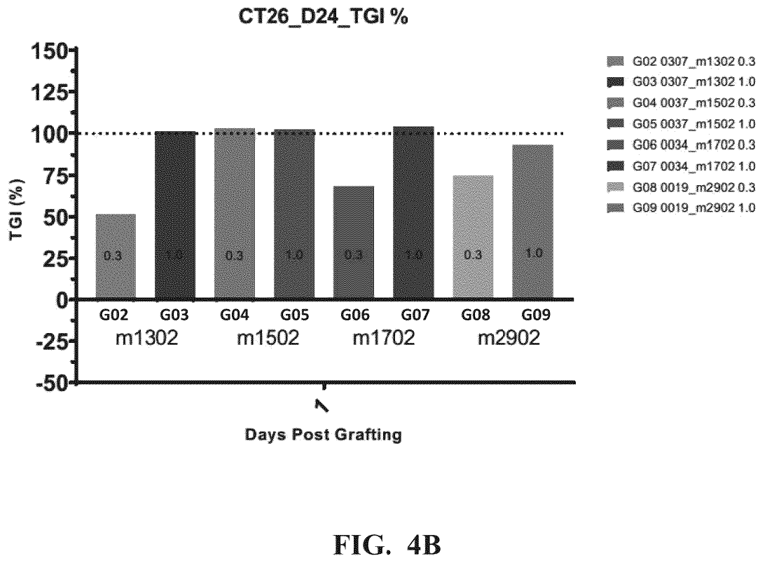

The exemplary agents that comprises a binder that binds to a target molecule, i.e., a T cell receptor beta variable region (TCRβV) binder, and a cytokine molecule, as provided herein, for example, m1302, m1502, m1702, and m2902, were administered at the dose of 0.3 mg/kg or 1.0 mg/kg once weekly to the mouse tumor model bearing the murine CT26 colon carcinoma cells on, for example, 6, 13, and 20 days post implant of the CT26 colon carcinoma cells, and the tumor volume was measured on several days post implant of the CT26 colon carcinoma cells, for example, on 6, 11, 13, 17, 20, 24, 27, 31, 34, and 38 days post implant of the CT26 colon carcinoma cells. As shown in FIG. 4A, administration of the exemplary agents that comprises a binder that binds to a target molecule, i.e., a T cell receptor beta variable region (TCRβV) binder, and a cytokine molecule, as provided herein, for example, m1302, m1502, m1702, and m2902, delayed tumor growth or reduced the tumor volume, relative to administration of vehicle control PBS (phosphate-buffered saline). For example, on 24 days post implant of the CT26 colon carcinoma cells, the percentage of the tumor growth inhibition (TGI %) by administration of m1302 at the dose of 1.0 mg/kg, administration of m1502 at the dose of 0.3 mg/kg and at the dose of 1.0 mg/kg, and administration of m1702 at the dose of 1.0 mg/kg was over 100% relative to administration of PBS (FIG. 4B). The percentage of tumor growth inhibition (TGI %) by administration of m2902 at the dose of 1.0 mg/kg was also close to 100% relative to administration of PBS, and administration of m1302 at the dose of 0.3 mg/kg, administration of m1702 at the dose of 0.3 mg/kg, and administration of m2902 at the dose of 0.3 mg/kg exhibited substantial tumor growth inhibition (FIG. 4B). The agent “m1302” (also referred as “mSTAR1302” or “Vβ13×IL-2”) comprises a binder that binds to mouse TCR Vβ13, and IL-2, and is a mouse surrogate for an agent that comprises a binder that binds to human TCR Vβ06, and IL-2 (referred as “V602”, “STAR0602”, or “Vβ06×IL-2”). The agent “m1502” (also referred as “mSTAR1502” or “Vβ15×IL-2”) comprises a binder that binds to mouse TCR Vβ15, and IL-2, and is a mouse surrogate for an agent that comprises a binder that binds to human TCR Vβ12, and IL-2 (referred as “1202”, “STAR1202”, or “Vβ12×IL-2”). The agent “m1702” (also referred as “mSTAR1702” or “Vβ17×IL-2”) comprises a binder that binds to mouse TCR Vβ17, and IL-2, and is a mouse surrogate for an agent that comprises a binder that binds to human TCR Vβ15, and IL-2 (referred as “1502”, “STAR1502”, or “Vβ15×IL-2”). The agent “m2902” (also referred as “mSTAR2902” or “Vβ29×IL-2”) comprises a binder that binds to mouse TCR Vβ29, and IL-2, and is a mouse surrogate for an agent that comprises a binder that binds to human TCR V028, and IL-2 (referred as “2802”, “STAR2802”, or “Vβ28×IL-2”). “0.3” indicates administration of the agent at the dose of 0.3 mg/kg, and “1.0” indicates administration of the agent at the dose of 1.0 mg/kg. The agents were administered once weekly (QW). The agents were administered by, for example, sc or ip.

### Example 4: Anti-Tumor Effects of the Exemplary Agents as Provided Herein, in the Mouse B16-F10 Tumor Model

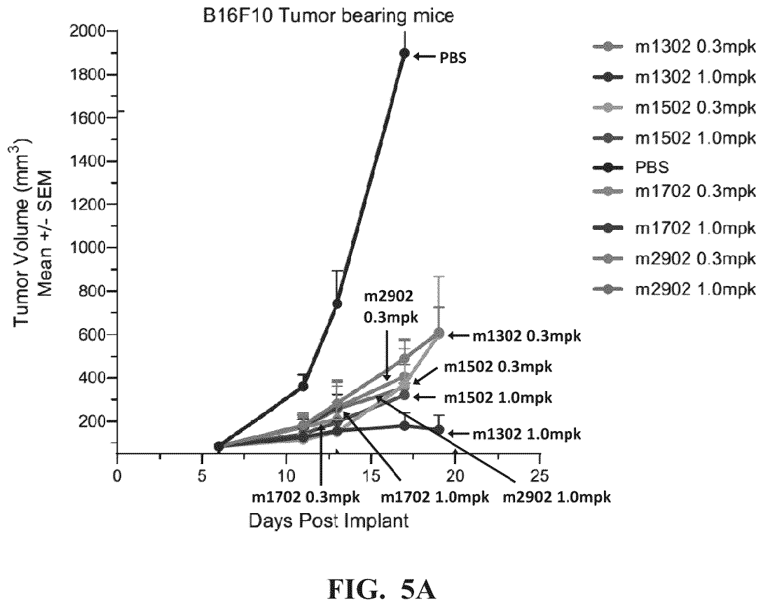

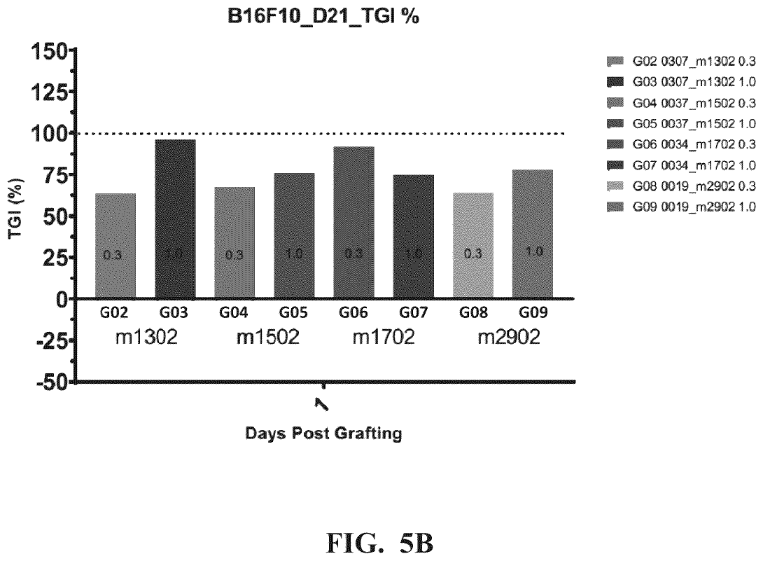

The exemplary agents that comprises a binder that binds to a target molecule, i.e., a T cell receptor beta variable region (TCRβV) binder, and a cytokine molecule, as provided herein, for example, m1302, m1502, m1702, and m2902, were administered at the dose of 0.3 mg/kg or 1.0 mg/kg once weekly to the mouse tumor model bearing the murine B16-F10 melanoma cells on, for example, 6, 13, and 20 days post implant of the B16-F10 melanoma cells, and the tumor volume was measured on several days post implant of the B16-F10 melanoma cells, for example, on 6, 11, 13, 17, and 19 days post implant of the B16-F10 melanoma cells. As shown in FIG. 5A, administration of the exemplary agents that comprises a binder that binds to a target molecule, i.e., a T cell receptor beta variable region (TCRβV) binder, and a cytokine molecule, as provided herein, for example, m1302, m1502, m1702, and m2902, delayed tumor growth or reduced the tumor volume, relative to administration of vehicle control PBS (phosphate-buffered saline). For example, FIG. 5B shows on 21 days post implant of the B16-F10 melanoma cells, administration of each of the agents as provided herein, for example, m1302, m1502, m1702, and m2902, exhibited substantial tumor growth inhibition. The agent “m1302” (also referred as “mSTAR1302” or “Vβ13×IL-2”) comprises a binder that binds to mouse TCR Vβ13, and IL-2, and is a mouse surrogate for an agent that comprises a binder that binds to human TCR Vβ06, and IL-2 (referred as “0602”, “STAR0602”, or “Vβ06×IL-2”). The agent “m1502” (also referred as “mSTAR1502” or “Vβ15×IL-2”) comprises a binder that binds to mouse TCR Vβ15, and IL-2, and is a mouse surrogate for an agent that comprises a binder that binds to human TCR Vβ12, and IL-2 (referred as “1202”, “STAR1202”, or “Vβ12×IL-2”). The agent “m1702” (also referred as “mSTAR1702” or “Vβ17×IL-2”) comprises a binder that binds to mouse TCR Vβ17, and IL-2, and is a mouse surrogate for an agent that comprises a binder that binds to human TCR Vβ15, and IL-2 (referred as “1502”, “STAR1502”, or “Vβ15×IL-2”). The agent “m2902” (also referred as “mSTAR2902” or “Vβ329×IL-2”) comprises a binder that binds to mouse TCR Vβ29, and IL-2, and is a mouse surrogate for an agent that comprises a binder that binds to human TCR Vβ28, and IL-2 (referred as “2802”, “STAR2802”, or “Vβ28×IL-2”). “0.3” indicates administration of the agent at the dose of 0.3 mg/kg, and “1.0” indicates administration of the agent at the dose of 1.0 mg/kg. The agents were administered once weekly (QW). The agents were administered by, for example, sc or ip.

### Example 5: Anti-Tumor Activity of the Exemplary Agents, as Provided Herein, in the Mouse MC38 Tumor Model

Multiple exemplary agents that comprises a binder that binds to a target molecule, i.e., a T cell receptor beta variable region (TCRβV) binder, and a cytokine molecule, as provided herein, i.e., Vβ-IL-2 surrogate murine constructs, were screened in a mouse MC38 tumor model of colorectal carcinoma with ˜3-7% baseline frequencies.

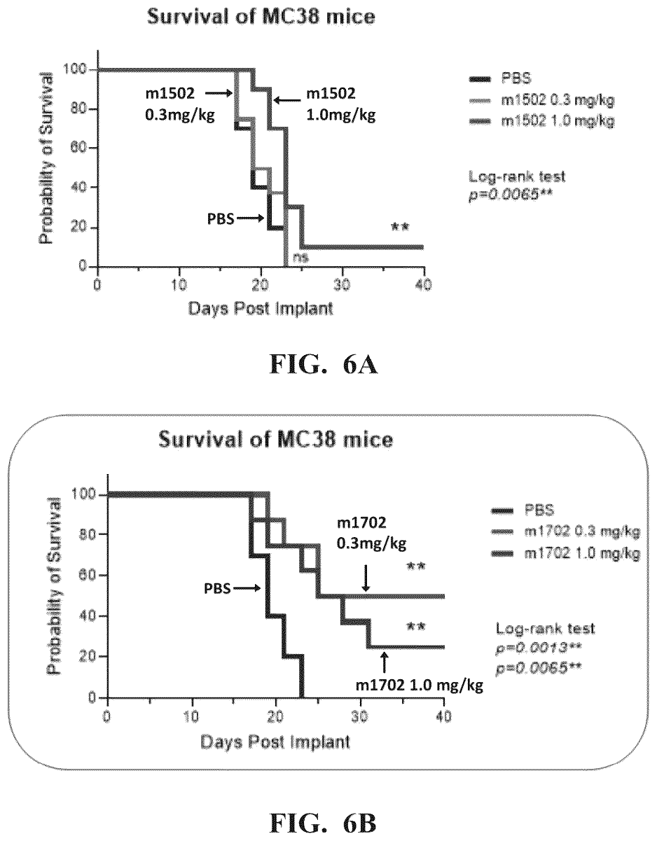

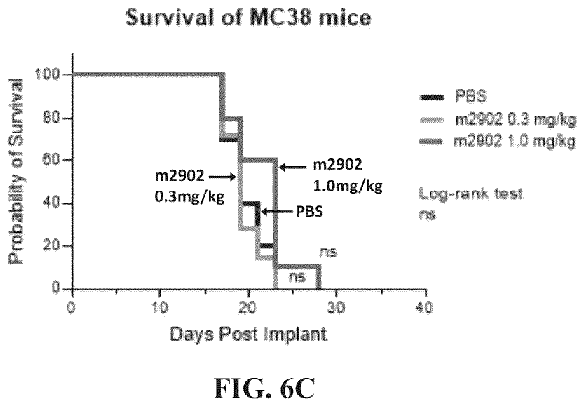

The exemplary agents that comprises a binder that binds to a target molecule, i.e., a T cell receptor beta variable region (TCRβV) binder, and a cytokine molecule, as provided herein, for example, m1502, m1702, and m2902, were administered at the dose of 0.3 mg/kg or 1.0 mg/kg once weekly to the mouse MC38 tumor model of colorectal carcinoma, and the probability of survival was assessed post implant of the MC38 colorectal carcinoma cells (FIG. 6A, FIG. 6B, FIG. 6C). Among these agents tested, mSTAR1702 (“Vβ17×IL-2”) exhibited similar efficacy as mSTAR1302 (Vβ13×IL-2) exhibited in a mouse MC38 tumor model. The agent “m1502” (also referred as “mSTAR1502” or “Vβ15×IL-2”) comprises a binder that binds to mouse TCR Vβ15, and IL-2, and is a mouse surrogate for an agent that comprises a binder that binds to human TCR Vβ12, and IL-2 (referred as “1202”, “STAR1202”, or “Vβ12×IL-2”), the agent “m1702” (also referred as “mSTAR1702” or “Vβ17×IL-2”) comprises a binder that binds to mouse TCR Vβ117, and IL-2, and is a mouse surrogate for an agent that comprises a binder that binds to human TCR Vβ15, and IL-2 (referred as “1502”, “STAR1502”, or “Vβ15×IL-2”). The agent “m2902” (also referred as “mSTAR2902” or “Vβ29×IL-2”) comprises a binder that binds to mouse TCR Vβ29, and IL-2, and is a mouse surrogate for an agent that comprises a binder that binds to human TCR Vβ28, and IL-2 (referred as “2802”, “STAR2802”, or “Vβ28×IL-2”). “0.3 mg/kg” indicates administration of the agent at the dose of 0.3 mg/kg, and “1.0 mg/kg” indicates administration of the agent at the dose of 1.0 mg/kg. The agents were administered by, for example, sc or ip.

### Example 6: Sequential Combination of the Exemplary Agents Reversed Tumor Stasis and Drove Complete Responses (CRs) to the Treatment in a Mouse MC38 Tumor Model

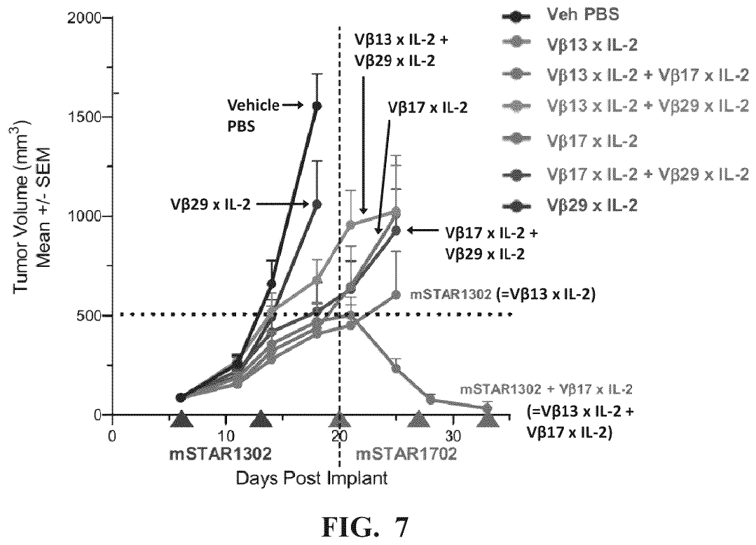

mSTAR1302 (“Vβ13×IL-2”), an exemplary first agent that comprises a first binder that binds to a first target molecule, i.e., a binder that binds to mouse TCRVβ13, and a cytokine molecule, i.e., IL-2, as provided herein, was administered at the dose of 1.0 mg/kg once weekly (QW) to a mouse MC38 tumor model of colorectal carcinoma on, for example, 6 and 13 days post implant of the MC38 colorectal carcinoma cells, followed by administration of mSTAR1702 (“Vβ17×IL-2”), an exemplary second agent that comprises a second binder that binds to a second target molecule, i.e., a binder that binds to mouse TCRVβ17, and a cytokine molecule, i.e., IL-2, as provided herein, at the dose of 1.0 mg/kg once weekly (QW) on, for example, 20, 27, and 34 days post implant of the MC38 colorectal carcinoma cells. In parallel, mSTAR1302 (“Vβ13×IL-2”) was administered at the dose of 1.0 mg/kg once weekly (QW) to a mouse MC38 tumor model of colorectal carcinoma on, for example, 6 and 13 days post implant of the MC38 colorectal carcinoma cells, followed by administration of mSTAR2902 (“Vβ29×IL-2”) at the dose of 1.0 mg/kg once weekly (QW) to a mouse MC38 tumor model on, for example, 20, 27, and 34 days post implant of the MC38 colorectal carcinoma cells, or mSTAR1702 (“Vβ17×IL-2”) was administered at the dose of 1.0 mg/kg once weekly (QW) to a mouse MC38 tumor model of colorectal carcinoma on, for example, 6 and 13 days post implant of the MC38 colorectal carcinoma cells, followed by administration of mSTAR2902 (“Vβ29×IL-2”) at the dose of 1.0 mg/kg once weekly (QW) to a mouse MC38 tumor model on, for example, 20, 27, and 34 days post implant of the MC38 colorectal carcinoma cells. Further, mSTAR1302 (“Vβ13×IL-2”) alone, mSTAR1702 (“Vβ17×IL-2”) alone, or mSTAR2902 (“Vβ29×IL-2”) alone was administered at the dose of 1.0 mg/kg once weekly (QW) to a mouse MC38 tumor model of colorectal carcinoma on, for example, 6, 13, 20, 27, and 34 days post implant of the MC38 colorectal carcinoma cells. The tumor volume was measured on several days post implant of the MC38 colorectal carcinoma cells, for example, on 6, 11, 13, 17, 21, 25, 28, and 33 days post implant of the MC38 colorectal carcinoma cells. As shown in FIG. 7, sequential administration of mSTAR1302 (“Vβ13×IL-2”), followed by administration of mSTAR1702 (“Vβ17×IL-2”) at the dose of 1.0 mg/kg once weekly (QW) to a mouse MC38 tumor model of colorectal carcinoma dramatically reduced the tumor volume, relative to administration of vehicle control PBS (phosphate-buffered saline), administration of mSTAR1302 (“Vβ13×IL-2”) alone, or administration of mSTAR1702 (“Vβ17×IL-2”) alone. For example, on 33 days post implant of MC38 colorectal carcinoma cells, the tumor almost completely disappeared by sequential administration of mSTAR1302 (“Vβ13×IL-2”) followed by administration of mSTAR1702 (“Vβ17×IL-2”). By comparison, sequential administration of mSTAR1302 (“Vβ13×IL-2”) followed by administration of mSTAR2902 (“Vβ29×IL-2”) or sequential administration of mSTAR1702 (“Vβ17×IL-2”) followed by administration of mSTAR2902 (“Vβ29×IL-2”) modestly reduced the tumor volume relative to administration of a single agent. The agent “mSTAR1302” (also referred as “m1302” or “Vβ13×IL-2”) comprises a binder that binds to mouse TCR Vβ13, and IL-2, and is a mouse surrogate for an agent that comprises a binder that binds to human TCR Vβ06, and IL-2 (referred as “0602”, “STAR0602”, or “Vβ06×IL-2”). The agent “mSTAR1702” (also referred as “m1702” or “Vβ17×IL-2”) comprises a binder that binds to mouse TCR Vβ17, and IL-2, and is a mouse surrogate for an agent that comprises a binder that binds to human TCR Vβ15, and IL-2 (referred as “1502”, “STAR1502”, or “Vβ15×IL-2”). The agent “Vβ29×IL-2” (also referred as “m2902” or “mSTAR2902”) comprises a binder that binds to mouse TCR Vβ29, and IL-2, and is a mouse surrogate for an agent that comprises a binder that binds to human TCR Vβ28, and IL-2 (referred as “2802”, “STAR2802”, or “Vβ28×IL-2”). The agents were administered by, for example, sc or ip.

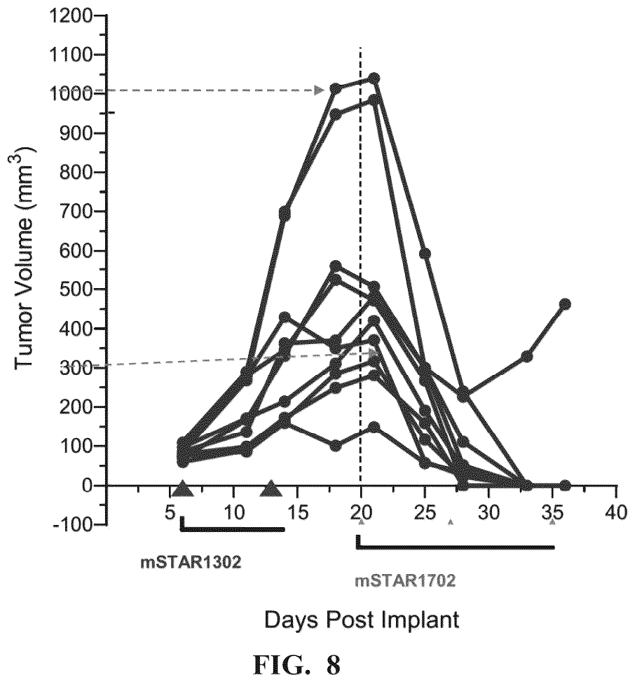

The anti-tumor activity of sequential administration of mSTAR1302 (“Vβ13×IL-2”), followed by administration of mSTAR1702 (“Vβ17×IL-2”) was further demonstrated in individual mice among total 10 mice of a mouse MC38 tumor model of colorectal carcinoma, mSTAR1302 (“Vβ13×IL-2”) was administered at the dose of 1.0 mg/kg once weekly (QW) to individual mice of a mouse MC38 tumor model of colorectal carcinoma on, for example, 6 and 13 days post implant of the MC38 colorectal carcinoma cells, followed by administration of mSTAR1702 (“Vβ17×IL-2”) at the dose of 1.0 mg/kg once weekly (QW) on, for example, 20, 27, and 35 days post implant of the MC38 colorectal carcinoma cells. The tumor volume was measured on several days post implant of the MC38 colorectal carcinoma cells, for example, on 6, 11, 14, 18, 21, 25, 28, 33, and 35 days post implant of the MC38 colorectal carcinoma cells. As shown in FIG. 8, 9 mice out of 10 mice exhibited tumor-free status by 33 days post implant of the MC38 colorectal carcinoma cells. Further, in some embodiments, addition of mSTAR1702 might appear to rescue anti-tumor activity in mSTAR1302-resistant tumors, as indicated by the upper arrow. Additionally, in some embodiments, addition of mSTAR1702 might also appear to drive furth anti-tumor activity in mice with stable disease (SD), as indicated by the lower arrow.

### Example 7: Concurrent Combination of the Exemplary Agents Did not Lead to Enhanced Anti-Tumor Activity in a Mouse MC38 Tumor Model

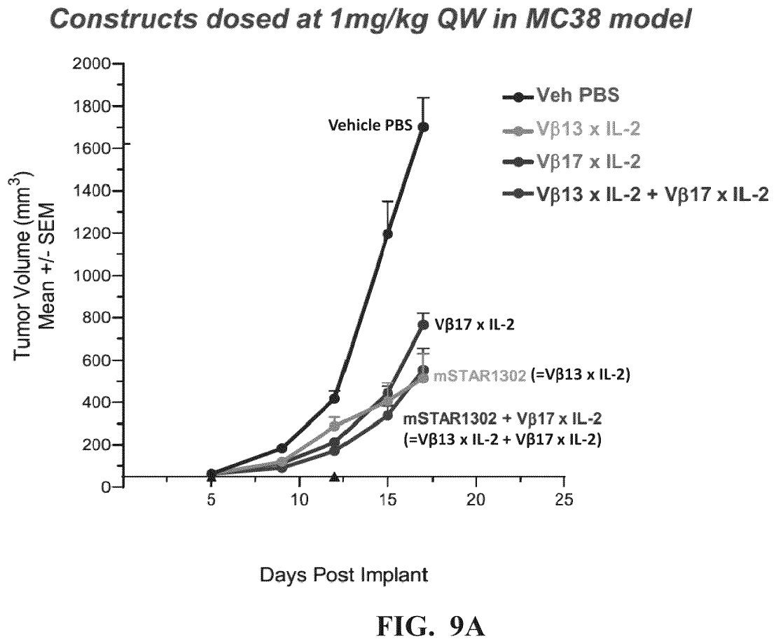

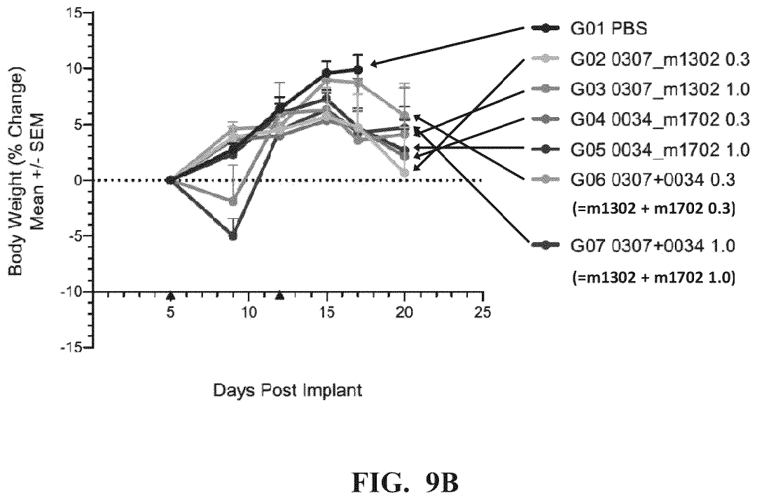

mSTAR1302 (“Vβ13×IL-2”), an exemplary first agent that comprises a first binder that binds to a first target molecule, i.e., a binder that binds to mouse TCRVβ13, and a cytokine molecule, i.e., IL-2, as provided herein, and Vβ17×IL-2 (mSTAR1702), an exemplary second agent that comprises a second binder that binds to a second target molecule, i.e., a binder that binds to mouse TCRVβ17, and a cytokine molecule, i.e., IL-2, as provided herein, were concurrently administered at the dose of 1.0 mg/kg once weekly (QW) in a mouse MC38 tumor model of colorectal carcinoma on, for example, 5 and 12 days post implant of the MC38 colorectal carcinoma cells. The tumor volume was measured on several days post implant of the MC38 colorectal carcinoma cells, for example, on 5, 9, 12, 15, and 17 days post implant of the MC38 colorectal carcinoma cells. As shown in FIG. 9A, concurrent administration of mSTAR1302 (“Vβ13×IL-2”) and Vβ17×IL-2 (mSTAR1702) did not lead to enhanced anti-tumor activity in the mouse MC38 tumor model of colorectal carcinoma. For example, concurrent administration of mSTAR1302 (“Vβ13×IL-2”) and Vβ17×IL-2 (mSTAR1702) did not further delay tumor growth or reduce the tumor volume in the mouse MC38 tumor model of colorectal carcinoma, relative to administration of mSTAR1302 (“Vβ13×IL-2”) alone or administration of Vβ17×IL-2 (mSTAR1702) alone (FIG. 9A). Further, mSTAR1302 (“Vβ13×IL-2”) and m1702 (“Vβ17×IL-2”) were concurrently administered at the dose of 0.3 mg/kg or 1 mg/kg once weekly (QW) to a mouse MC38 tumor model of colorectal carcinoma on, for example, 5 and 12 days post implant of the MC38 colorectal carcinoma cells, and the body weight of the mice were measured on, for example, 5, 9, 12, 15, 17, and 20 days post implant of the MC38 colorectal carcinoma cells. As shown in FIG. 9B, concurrent administration of mSTAR1302 (“Vβ13×IL-2”) and m1702 (“Vβ17×IL-2”) had minimal impact on the body weight at the dose of 0.3 mg/kg or 1 mg/kg in the mouse MC38 tumor model of colorectal carcinoma, compared with administration of vehicle control PBS (phosphate-buffered saline), administration of mSTAR1302 (“V13×IL-2”) alone, or administration of m1702 (“Vβ17×IL-2”) alone. The agent “m1302” (also referred as “mSTAR1302” or “Vβ13×IL-2”) comprises a binder that binds to mouse TCR Vβ13, and IL-2, and is a mouse surrogate for an agent that comprises a binder that binds to human TCR Vβ06, and IL-2 (referred as “0602”, “STAR0602”, or “Vβ06×IL-2”). The agent “m1702”, also referred as “mSTAR1702” or “Vβ17×IL-2”, comprises a binder that binds to mouse TCR Vβ7, and IL-2, and is a mouse surrogate for an agent that comprises a binder that binds to human TCR Vβ15, and IL-2 (referred as “1502”, “STAR1502”, or “Vβ15×IL-2”). “0.3” indicates administration of the agent at the dose of 0.3 mg/kg, and “1.0” indicates administration of the agent at the dose of 1.0 mg/kg. The agents were administered once weekly.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby. Those skilled in the art will recognize, or be able to ascertain using no more than routine experimentation, many equivalents to the specific embodiments of the invention described herein. Such equivalents are intended to be encompassed by the following claims.

What is claimed is:

1. A method of treating a disease or condition in a subject in need thereof comprising administering a first agent that comprises a first binder that binds to a first target molecule to the subject, and administering a second agent that comprises a second binder that binds to a second target molecule to the subject, thereby treating the disease or condition in the subject;
wherein an administration of the first agent is followed by an administration of the second agent;
wherein the first target molecule and the second target molecule are independently selected from the group consisting of a T cell receptor alpha variable region (TCRαV) and a T cell receptor beta variable region (TCRβV); and
wherein the first target molecule and the second target molecule are different.

2. The method of claim 1, wherein the first agent and the second agent are not concomitantly administered to the subject.

3. The method of claim 1, wherein administering the first agent comprises administering a therapeutically effective amount of the first agent to the subject.

4.-570. (canceled)

571. The method of claim 1, wherein administering the second agent comprises administering a therapeutically effective amount of the second agent to the subject.

572. The method of claim 1, wherein the method is more effective to treat the disease or condition in the subject relative to (i) a method consisting of administering the first agent to the subject but not administering the second agent to the subject, or (ii) a method consisting of administering the second agent to the subject but not administering the first agent to the subject.

573. The method of claim 1, wherein:
(i) the method comprises administering two or more doses of the first agent and/or two or more doses of the second agent;
(ii) the method comprises administering at least one dose of the first agent per week and/or at least one dose of the second agent per week; or
(iii) the method comprises administering the first agent to the subject once every two, three, four, six, eight, or twelve weeks and/or administering the second agent to the subject once every two, three, four, six, eight, or twelve weeks.

574. The method of claim 1, wherein the first agent is administered to the subject at least one week prior to administering the second agent.

575. The method of claim 1, wherein the first agent and the second agent are administered to the subject according to a dosing regimen,
wherein the dosing regimen comprises an administration of the first agent once a week for two, three, four, six, eight, or twelve weeks followed by an administration of the second agent once a week for two, three, four, six, eight, or twelve weeks;
wherein the second agent is administered to the subject one week after the last administration of the first agent; and
wherein the dosing regimen is repeated twice or more.

576. The method of claim 1, wherein:
(i) the administration of the first agent comprises two or more doses, and each dose of the first agent comprises a same amount of the first agent; and/or
(ii) the administration of the second agent comprises two or more doses, and each dose of the second agent comprises a same amount of the second agent.

577. The method of claim 1, wherein:
(i) the first agent is administered to the subject at a dose of from about 0.001 mg/kg to about 10 mg/kg and the second agent is administered to the subject at a dose of from about 0.001 mg/kg to about 10 mg/kg; or
(ii) the first agent is administered to the subject at a dose of about 0.3 mg/kg or about 1 mg/kg and the second agent is administered to the subject at a dose of from about 0.3 mg/kg or about 1 mg/kg.

578. The method of claim 1, wherein:
(i) the first target molecule is a first TCRβV, and the second target molecule is a second TCRβV, wherein the first TCRβV and the second TCRβV are different;
(ii) the first target molecule is a first TCRαV, and the second target molecule is a second TCRαV, wherein the first TCRαV and the second TCRαV are different;
(iii) the first target molecule is a TCRβV, and the second target molecule is a TCRαV; or
(iv) the first target molecule is a TCRαV, and the second target molecule is a TCRβV.

579. The method of claim 1, wherein the first target molecule and the second target molecule are different, and
wherein:
(a) the first target molecule is human TRBV5, human TRBV6, human TRBV10, human TRBV7, human TRBV15, human TRBV19, human TRBV20, human TRBV28, human TRAV12, human TRAV13, human TRAV19, human TRAV21, or human TRAV30; and the second target molecule is human TRBV5, human TRBV6, human TRBV10, human TRBV7, human TRBV15, human TRBV19, human TRBV20, human TRBV28, human TRAV12, human TRAV13, human TRAV19, human TRAV21, or human TRAV30, or
(b) the first target molecule is human TRBV5-1, human TRBV6-5, human TRBV10-1, human TRBV10-2, human TRBV10-3, human TRBV7-2, human TRBV7-3, human TRBV19, human TRBV15, human TRBV20-1, human TRBV27, human TRBV28, human TRAV12-2, human TRAV13-1, human TRAV19, human TRAV21, or human TRAV30; and the second target molecule is human TRBV5-1, human TRBV6-5, human TRBV10-1, human TRBV10-2, human TRBV10-3, human TRBV7-2, human TRBV7-3, human TRBV19, human TRBV15, human TRBV20-1, human TRBV27, human TRBV28, human TRAV12-2, human TRAV13-1, human TRAV19, human TRAV21, or human TRAV30.

580. The method of claim 1, wherein the first binder and/or the second binder comprises a full antibody or an antigen-binding fragment thereof, a bivalent antibody, a bispecific antibody, or a biparatopic antibody,
wherein the antigen-binding fragment is a Fab, F(ab′)2, Fv, a single chain Fv (scFv), a diabody, a single domain antibody, or a camelid antibody.

581. The method of claim 1, wherein the first binder and the second binder bind to different TCRβVs, wherein the different TCRβVs belong to the same TCRβV subfamily.

582. The method of claim 1, wherein the first binder and/or the second binder comprises a heavy chain variable region (VH) comprising:
(a) a HC CDR1 comprising the sequence of SEQ ID NO: 3351, a HC CDR2 comprising the sequence of SEQ ID NO: 3352, and a HC CDR3 comprising the sequence of SEQ ID NO: 3353;
(b) a HC CDR1 comprising the sequence of SEQ ID NO: 3351, SEQ ID NO: 3421, or SEQ ID NO: 3702, a HC CDR2 comprising the sequence of SEQ ID NO: 3352, and a HC CDR3 comprising the sequence of SEQ ID NO: 3353;
(c) a HC CDR1 comprising the sequence of SEQ ID NO: 3341, a HC CDR2 comprising the sequence of SEQ ID NO: 3342, and a HC CDR3 comprising the sequence of SEQ ID NO: 3343;
(d) a HC CDR1 comprising the sequence of SEQ ID NO: 3321, a HC CDR2 comprising the sequence of SEQ ID NO: 3322, and a HC CDR3 comprising the sequence of SEQ ID NO: 3323;
(e) a HC CDR1 comprising the sequence of SEQ ID NO: 1190, a HC CDR2 comprising the sequence of SEQ ID NO: 1186, and a HC CDR3 comprising the sequence of SEQ ID NO: 1187;
(f) a HC CDR1 comprising the sequence of SEQ ID NO: 3371, a HC CDR2 comprising the sequence of SEQ ID NO: 3372, and a HC CDR3 comprising the sequence of SEQ ID NO: 3373;
(g) a HC CDR1 comprising the sequence of SEQ ID NO: 3381, a HC CDR2 comprising the sequence of SEQ ID NO: 3382, and a HC CDR3 comprising the sequence of SEQ ID NO: 3383;
(h) a HC CDR1 comprising the sequence of SEQ ID NO: 3391, a HC CDR2 comprising the sequence of SEQ ID NO: 3392, and a HC CDR3 comprising the sequence of SEQ ID NO: 3393;
(i) a HC CDR1 comprising the sequence of SEQ ID NO: 3401, a HC CDR2 comprising the sequence of SEQ ID NO: 3402, and a HC CDR3 comprising the sequence of SEQ ID NO: 3403; or
(j) a HC CDR1 comprising the sequence of SEQ ID NO: 3411, a HC CDR2 comprising the sequence of SEQ ID NO: 3412, and a HC CDR3 comprising the sequence of SEQ ID NO: 3413; and
wherein the first binder and/or the second binder comprises a light chain variable region (VL) comprising:
(i) a LC CDR1 comprising the sequence of SEQ ID NO: 3344, a LC CDR2 comprising the sequence of SEQ ID NO: 3345, and a LC CDR3 comprising the sequence of SEQ ID NO: 3346;
(ii) a LC CDR1 comprising the sequence of SEQ ID NO: 3324, a LC CDR2 comprising the sequence of SEQ ID NO: 3325, and a LC CDR3 comprising the sequence of SEQ ID NO: 3326;
(iii) a LC CDR1 comprising the sequence of SEQ ID NO: 3354, a LC CDR2 comprising the sequence of SEQ ID NO: 3355, and a LC CDR3 comprising the sequence of SEQ ID NO: 3356;
(iv) a LC CDR1 comprising the sequence of SEQ ID NO: 1191, a LC CDR2 comprising the sequence of SEQ ID NO: 1192, and a LC CDR3 comprising the sequence of SEQ ID NO: 1193;
(v) a LC CDR1 comprising the sequence of SEQ ID NO: 3374, a LC CDR2 comprising the sequence of SEQ ID NO: 3375, and a LC CDR3 comprising the sequence of SEQ ID NO: 3376;
(vi) a LC CDR1 comprising the sequence of SEQ ID NO: 3384, a LC CDR2 comprising the sequence of SEQ ID NO: 3325, and a LC CDR3 comprising the sequence of SEQ ID NO: 3386;
(vii) a LC CDR1 comprising the sequence of SEQ ID NO: 3394, a LC CDR2 comprising the sequence of SEQ ID NO: 3395, and a LC CDR3 comprising the sequence of SEQ ID NO: 3396;
(viii) a LC CDR1 comprising the sequence of SEQ ID NO: 3404, a LC CDR2 comprising the sequence of SEQ ID NO: 3405, and a LC CDR3 comprising the sequence of SEQ ID NO: 3406;
(ix) a LC CDR1 comprising the sequence of SEQ ID NO: 3414, a LC CDR2 comprising the sequence of SEQ ID NO: 3415, and a LC CDR3 comprising the sequence of SEQ ID NO: 3416; or
(x) a LC CDR1 comprising the sequence of SEQ ID NO: 3354, a LC CDR2 comprising the sequence of SEQ ID NO: 3443, and a LC CDR3 comprising the sequence of SEQ ID NO: 3356.

583. The method of claim 1, wherein the first binder and/or the second binder comprises:
(i) a VH comprising a HC CDR1 comprising the sequence of SEQ ID NO: 3351, a HC CDR2 comprising the sequence of SEQ ID NO: 3352, a HC CDR3 comprising the sequence of SEQ ID NO: 3353, and a VL comprising a LC CDR1 comprising the sequence of SEQ ID NO: 3354, a LC CDR2 comprising the sequence of SEQ ID NO: 3355, and a LC CDR3 comprising the sequence of SEQ ID NO: 3356;
(ii) a VH comprising a HC CDR1 comprising the sequence of SEQ ID NO: 3351, SEQ ID NO: 3421, or SEQ ID NO: 3702, a HC CDR2 comprising the sequence of SEQ ID NO: 3352, a HC CDR3 comprising the sequence of SEQ ID NO: 3353, and a VL comprising a LC CDR1 comprising the sequence of SEQ ID NO: 3354, a LC CDR2 comprising the sequence of SEQ ID NO: 3355, and a LC CDR3 comprising the sequence of SEQ ID NO: 3356;
(iii) a VH comprising a HC CDR1 comprising the sequence of SEQ ID NO: 3341, a HC CDR2 comprising the sequence of SEQ ID NO: 3342, a HC CDR3 comprising the sequence of SEQ ID NO: 3343, and a VL comprising a LC CDR1 comprising the sequence of SEQ ID NO: 3344, a LC CDR2 comprising the sequence of SEQ ID NO: 3345, and a LC CDR3 comprising the sequence of SEQ ID NO: 3346;
(iv) a VH comprising a HC CDR1 comprising the sequence of SEQ ID NO: 3321, a HC CDR2 comprising the sequence of SEQ ID NO: 3322, a HC CDR3 comprising the sequence of SEQ ID NO: 3323, and a VL comprising a LC CDR1 comprising the sequence of SEQ ID NO: 3324, a LC CDR2 comprising the sequence of SEQ ID NO: 3325, and a LC CDR3 comprising the sequence of SEQ ID NO: 3326;
(v) a VH comprising a HC CDR1 comprising the sequence of SEQ ID NO: 1190, a HC CDR2 comprising the sequence of SEQ ID NO: 1186, a HC CDR3 comprising the sequence of SEQ ID NO: 1187, and a VL comprising a LC CDR1 comprising the sequence of SEQ ID NO: 1191, a LC CDR2 comprising the sequence of SEQ ID NO: 1192, and a LC CDR3 comprising the sequence of SEQ ID NO: 1193;
(vi) a VH comprising a HC CDR1 comprising the sequence of SEQ ID NO: 3371, a HC CDR2 comprising the sequence of SEQ ID NO: 3372, a HC CDR3 comprising the sequence of SEQ ID NO: 3373, and a VL comprising a LC CDR1 comprising the sequence of SEQ ID NO: 3374, a LC CDR2 comprising the sequence of SEQ ID NO: 3375, and a LC CDR3 comprising the sequence of SEQ ID NO: 3376;
(vii) a VH comprising a HC CDR1 comprising the sequence of SEQ ID NO: 3381, a HC CDR2 comprising the sequence of SEQ ID NO: 3382, a HC CDR3 comprising the sequence of SEQ ID NO: 3383, and a VL comprising a LC CDR1 comprising the sequence of SEQ ID NO: 3384, a LC CDR2 comprising the sequence of SEQ ID NO: 3325, and a LC CDR3 comprising the sequence of SEQ ID NO: 3386;
(viii) a VH comprising a HC CDR1 comprising the sequence of SEQ ID NO: 3391, a HC CDR2 comprising the sequence of SEQ ID NO: 3392, a HC CDR3 comprising the sequence of SEQ ID NO: 3393, and a VL comprising a LC CDR1 comprising the sequence of SEQ ID NO: 3394, a LC CDR2 comprising the sequence of SEQ ID NO: 3395, and a LC CDR3 comprising the sequence of SEQ ID NO: 3396;
(ix) a VH comprising a HC CDR1 comprising the sequence of SEQ ID NO: 3401, a HC CDR2 comprising the sequence of SEQ ID NO: 3402, a HC CDR3 comprising the sequence of SEQ ID NO: 3403, and a VL comprising a LC CDR1 comprising the sequence of SEQ ID NO: 3404, a LC CDR2 comprising the sequence of SEQ ID NO: 3405, and a LC CDR3 comprising the sequence of SEQ ID NO: 3406;
(x) a VH comprising a HC CDR1 comprising the sequence of SEQ ID NO: 3411, a HC CDR2 comprising the sequence of SEQ ID NO: 3412, a HC CDR3 comprising the sequence of SEQ ID NO: 3413, and a VL comprising a LC CDR1 comprising the sequence of SEQ ID NO: 3414, a LC CDR2 comprising the sequence of SEQ ID NO: 3415, and a LC CDR3 comprising the sequence of SEQ ID NO: 3416; or
(xi) a VH comprising a HC CDR1 comprising the sequence of SEQ ID NO: 3351, SEQ ID NO: 3421, or SEQ ID NO: 3702, a HC CDR2 comprising the sequence of SEQ ID NO: 3352, a HC CDR3 comprising the sequence of SEQ ID NO: 3353, and a VL comprising a LC CDR1 comprising the sequence of SEQ ID NO: 3354, a LC CDR2 comprising the sequence of SEQ ID NO: 3443, and a LC CDR3 comprising the sequence of SEQ ID NO: 3356.

584. The method of claim 583, wherein:
(A)(i) the VH comprises a sequence having at least 75% sequence identity to any one sequence selected from the group consisting of: SEQ ID NO: 1346, SEQ ID NO: 3445, SEQ ID NO: 3448, SEQ ID NO: 3450, SEQ ID NO: 1196, SEQ ID NO: 3455, SEQ ID NO: 3457, SEQ ID NO: 3463, SEQ ID NO: 3465, and SEQ ID NO: 3470; and/or
(ii) the VL comprises a sequence having at least 75% sequence identity to any one sequence selected from the group consisting of: SEQ ID NO: 1349, SEQ ID NO: 3446, SEQ ID NO: 3449, SEQ ID NO: 3451, SEQ ID NO:1195, SEQ ID NO: 3456, SEQ ID NO: 3458, SEQ ID NO: 3464, SEQ ID NO: 3466, SEQ ID NO: 3471, SEQ ID NO: 3475, and SEQ ID NO: 3476;
(B)(i) the VH comprises a sequence having at least 75% sequence identity to the sequence of SEQ ID NO: 1346, and the VL comprises a sequence having at least 75% sequence identity to the sequence of SEQ ID NO: 1349;
(ii) the VH comprises a sequence having at least 75% sequence identity to the sequence of SEQ ID NO: 3445, and the VL comprises a sequence having at least 75% sequence identity to the sequence of SEQ ID NO: 3446;
(iii) the VH comprises a sequence having at least 75% sequence identity to the sequence of SEQ ID NO: 3448, and the VL comprises a sequence having at least 75% sequence identity to the sequence of SEQ ID NO: 3449;
(iv) the VH comprises a sequence having at least 75% sequence identity to the sequence of SEQ ID NO: 3450, and the VL comprises a sequence having at least 75% sequence identity to the sequence of SEQ ID NO: 3451;
(v) the VH comprises a sequence having at least 75% sequence identity to the sequence of SEQ ID NO: 1196, and the VL comprises a sequence having at least 75% sequence identity to the sequence of SEQ ID NO: 1195;
(vi) the VH comprises a sequence having at least 75% sequence identity to the sequence of SEQ ID NO: 3455, and the VL comprises a sequence having at least 75% sequence identity to the sequence of SEQ ID NO: 3456;
(vii) the VH comprises a sequence having at least 75% sequence identity to the sequence of SEQ ID NO: 3457, and the VL comprises a sequence having at least 75% sequence identity to the sequence of SEQ ID NO: 3458;
(viii) the VH comprises a sequence having at least 75% sequence identity to the sequence of SEQ ID NO: 3463, and the VL comprises a sequence having at least 75% sequence identity to the sequence of SEQ ID NO: 3464;
(ix) the VH comprises a sequence having at least 75% sequence identity to the sequence of SEQ ID NO: 3465, and the VL comprises a sequence having at least 75% sequence identity to the sequence of SEQ ID NO: 3466;
(x) the VH comprises a sequence having at least 75% sequence identity to the sequence of SEQ ID NO: 3470, and the VL comprises a sequence having at least 75% sequence identity to the sequence of SEQ ID NO: 3471;
(xi) the VH comprises a sequence having at least 75% sequence identity to the sequence of SEQ ID NO: 1346, and the VL comprises a sequence having at least 75% sequence identity to the sequence of SEQ ID NO: 3475; or
(xii) the VH comprises a sequence having at least 75% sequence identity to the sequence of SEQ ID NO: 1346, and the VL comprises a sequence having at least 75% sequence identity to the sequence of SEQ ID NO: 3476; or
(C)(i) the VH comprises the sequence of SEQ ID NO: 1346, and the VL comprises the sequence of SEQ ID NO: 1349;
(ii) the VH comprises the sequence of SEQ ID NO: 3445, and the VL comprises the sequence of SEQ ID NO: 3446;
(iii) the VH comprises the sequence of SEQ ID NO: 3448, and the VL comprises the sequence of SEQ ID NO: 3449;
(iv) the VH comprises the sequence of SEQ ID NO: 3450, and the VL comprises the sequence of SEQ ID NO: 3451;
(v) the VH comprises the sequence of SEQ ID NO: 1196, and the VL comprises the sequence of SEQ ID NO: 1195;
(vi) the VH comprises the sequence of SEQ ID NO: 3455, and the VL comprises the sequence of SEQ ID NO: 3456;
(vii) the VH comprises the sequence of SEQ ID NO: 3457, and the VL comprises the sequence of SEQ ID NO: 3458;
(viii) the VH comprises the sequence of SEQ ID NO: 3463, and the VL comprises the sequence of SEQ ID NO: 3464;
(ix) the VH comprises the sequence of SEQ ID NO: 3465, and the VL comprises the sequence of SEQ ID NO: 3466;
(x) the VH comprises the sequence of SEQ ID NO: 3470, and the VL comprises the sequence of SEQ ID NO: 3471;
(xi) the VH comprises the sequence of SEQ ID NO: 1346, and the VL comprises the sequence of SEQ ID NO: 3475; or
(xii) the VH comprises the sequence of SEQ ID NO: 1346, and the VL comprises the sequence of SEQ ID NO: 3476.

585. The method of claim 1, wherein the first binder and/or the second binder comprises:
(i) one or more heavy chain constant regions selected from the group consisting of IgG1 heavy chain constant region or fragment thereof, IgG2 heavy chain constant region or fragment thereof, IgG3 heavy chain constant region or fragment thereof, IgGA1 heavy chain constant region or fragment thereof, IgGA2 heavy chain constant region or fragment thereof, IgG4 heavy chain constant region or fragment thereof, IgJ heavy chain constant region or fragment thereof, IgM heavy chain constant region or fragment thereof, IgD heavy chain constant region or fragment thereof, and IgE heavy chain constant region or fragment thereof, and/or
(ii) a kappa light chain constant region or fragment thereof, a lambda light chain constant region or fragment thereof, or a combination thereof.

586. The method of claim 1, wherein the first agent and/or the second agent is a multifunctional molecule and/or a multispecific molecule.

587. The method of claim 1, wherein the first agent and/or the second agent comprises at least two non-contiguous polypeptide chains comprising a first polypeptide chain and a second polypeptide chain,
wherein the first polypeptide chain comprises a first Fc region, and the second polypeptide chain comprises a second Fc region; and
wherein the first Fc region and the second Fc region comprise an Fc interface with a knob-in-a hole.

588. The method of claim 587, wherein the first Fc region, the second Fc region, or a combination thereof comprises an Asn297Ala mutation, a Leu234Ala/Leu235Ala mutation, or a combination thereof.

589. The method of claim 1, wherein the first agent and/or the second agent is a multispecific molecule that further comprises one or more of a tumor-targeting moiety, a cytokine molecule, or an immune cell engager,
wherein the immune cell engager is selected from the group consisting of a T cell engager, an NK cell engager, a B cell engager, a dendritic cell engager, a macrophage cell engager, and any combination thereof, and
wherein the cytokine molecule is selected from the group consisting of interleukin-2 (IL-2) or a functional variant thereof, interleukin-7 (IL-7) or a functional variant thereof, interleukin-12 (IL-12) or a functional variant thereof, interleukin-15 (IL-15) or a functional variant thereof, interleukin-18 (IL-18) or a functional variant thereof, interleukin-21 (IL-21) or a functional variant thereof, interferon gamma or a functional variant thereof, and any combination thereof.

590. The method of claim 589, wherein the cytokine molecule comprises IL-2 or a functional variant thereof,
wherein the IL-2 comprises the sequence of SEQ ID NO: 2270 or SEQ ID NO: 2191.

591. The method of claim 589, wherein the cytokine molecule comprises IL-15 or a functional variant thereof,
wherein the IL-15 comprises the sequence of SEQ ID NO: 2170.

592. The method of claim 591, wherein the IL-15 or a functional variant thereof is covalently linked to an IL15Ralpha dimerizing domain.

593. The method of claim 592, wherein the IL15Ralpha dimerizing domain comprises an IL-15 receptor alpha sushi domain or a functional variant thereof,
wherein the IL15Ralpha dimerizing domain comprises the sequence of SEQ ID NO: 3472; and
wherein the cytokine molecule comprises the sequence of SEQ ID NO: 3474.

594. The method of claim 589, wherein the cytokine molecule comprises IL-21 or a functional variant thereof,
wherein the IL-21 comprises the sequence of SEQ ID NO: 2193.

595. The method of claim 589, wherein the cytokine molecule comprises IL-12 or a functional variant thereof,
wherein the IL-12 comprises the sequence of SEQ ID NO: 2290.

596. The method of claim 589, wherein the tumor-targeting moiety binds to Her2/neu.

597. The method of claim 589, wherein the tumor-targeting moiety binds to CD19.

598. The method of claim 589, wherein the tumor-targeting moiety binds to CD123.

599. The method of claim 1, wherein the first agent and/or the second agent comprises a first polypeptide chain, a second polypeptide chain, and a third polypeptide chain, wherein the first polypeptide chain, the second polypeptide chain, and the third polypeptide chain are non-contiguous, and
wherein:
(A)(i) the first polypeptide chain comprises a VH comprising the sequence of SEQ ID NO: 1346, wherein the VH is linked to a heavy chain constant region comprising the sequence of SEQ ID NO: 3452 or the sequence of SEQ ID NO: 3504, wherein the heavy chain constant region comprises the first Fc region comprising the sequence of SEQ ID NO: 3453 or the sequence of SEQ ID NO: 3505,
(ii) the second polypeptide chain comprises a cytokine molecule, wherein the cytokine molecule is linked to the second Fc region comprising the sequence of SEQ ID NO: 3454 or the sequence of SEQ ID NO: 3501, wherein the cytokine molecule comprises IL-2 or a functional variant thereof comprising the sequence of SEQ ID NO: 2270, and
(iii) the third polypeptide chain comprises a VL comprising the sequence of SEQ ID NO: 1349, wherein the VL is linked to a light chain constant region comprising the sequence of SEQ ID NO: 3644;
(B)(i) the first polypeptide chain comprises a VH comprising the sequence of SEQ ID NO: 1346, wherein the VH is linked to a heavy chain constant region comprising the sequence of SEQ ID NO: 3452 or the sequence of SEQ ID NO: 3504, wherein the heavy chain constant region comprises the first Fc region comprising the sequence of SEQ ID NO: 3453 or the sequence of SEQ ID NO: 3505,
(ii) the second polypeptide chain comprises a cytokine molecule, wherein the cytokine molecule is linked to the second Fc region comprising the sequence of SEQ ID NO: 3648 or SEQ ID NO: 3454, wherein the cytokine molecule comprises IL-15 or a functional variant thereof comprising the sequence of SEQ ID NO: 2170, wherein the IL-15 or a functional variant thereof is covalently linked to an IL15Ralpha dimerizing domain comprising the sequence of the SEQ ID NO: 3472, and
(iii) the third polypeptide chain comprises a VL comprising the sequence of SEQ ID NO: 1349, wherein the VL is linked to a light chain constant region comprising the sequence of SEQ ID NO: 3644;
(C)(i) the first polypeptide chain comprises a VH comprising the sequence of SEQ ID NO: 3445, wherein the VH is linked to a heavy chain constant region comprising the sequence of SEQ ID NO: 3649 or SEQ ID NO: 3452, wherein the heavy chain constant region comprises the first Fc region comprising the sequence of SEQ ID NO: 3447 or SEQ ID NO: 3453,
(ii) the second polypeptide chain comprises a cytokine molecule, wherein the cytokine molecule is linked to the second Fc region comprising the sequence of SEQ ID NO: 3648 or SEQ ID NO: 3454, wherein the cytokine molecule comprises IL-2 or a functional variant thereof comprising the sequence of SEQ ID NO: 2270, and
(iii) the third polypeptide chain comprises a VL comprising the sequence of SEQ ID NO: 3446, wherein the VL is linked to a light chain constant region comprising the sequence of SEQ ID NO: 3644;
(D)(i) the first polypeptide chain comprises a VH comprising the sequence of SEQ ID NO: 3448, wherein the VH is linked to a heavy chain constant region comprising the sequence of SEQ ID NO: 3649 or SEQ ID NO: 3452, wherein the heavy chain constant region comprises the first Fc region comprising the sequence of SEQ ID NO: 3447 or SEQ ID NO: 3453,
(ii) the second polypeptide chain comprises a cytokine molecule, wherein the cytokine molecule is linked to the second Fc region comprising the sequence of SEQ ID NO: 3648 or SEQ ID NO: 3454, wherein the cytokine molecule comprises IL-2 or a functional variant thereof comprising the sequence of SEQ ID NO: 2270, and
(iii) the third polypeptide chain comprises a VL comprising the sequence of SEQ ID NO: 3449, wherein the VL is linked to a light chain constant region comprising the sequence of SEQ ID NO: 3644;
(E)(i) the first polypeptide chain comprises a VH comprising the sequence of SEQ ID NO: 3450, wherein the VH is linked to a heavy chain constant region comprising the sequence of SEQ ID NO: 3649 or SEQ ID NO: 3452, wherein the heavy chain constant region comprises the first Fc region comprising the sequence of SEQ ID NO: 3447 or SEQ ID NO: 3453,
(ii) the second polypeptide chain comprises a cytokine molecule, wherein the cytokine molecule is linked to the second Fc region comprising the sequence of SEQ ID NO: 3648 or SEQ ID NO: 3454, wherein the cytokine molecule comprises IL-2 or a functional variant thereof comprising the sequence of SEQ ID NO: 2270, and
(iii) the third polypeptide chain comprises a VL comprising the sequence of SEQ ID NO: 3451, wherein the VL is linked to a light chain constant region comprising the sequence of SEQ ID NO: 3644;
(F)(i) the first polypeptide chain comprises a VH comprising the sequence of SEQ ID NO: 3450, wherein the VH is linked to a heavy chain constant region comprising the sequence of SEQ ID NO: 3649 or SEQ ID NO: 3452, wherein the heavy chain constant region comprises the first Fc region comprising the sequence of SEQ ID NO: 3447 or SEQ ID NO: 3453,
(ii) the second polypeptide chain comprises a cytokine molecule, wherein the cytokine molecule is linked to the second Fc region comprising the sequence of SEQ ID NO: 3454 or the sequence of SEQ ID NO: 3501, wherein the cytokine molecule comprises IL-2 or a functional variant thereof comprising the sequence of SEQ ID NO: 2270, and
(iii) the third polypeptide chain comprises a VL comprising the sequence of SEQ ID NO: 3451, wherein the VL is linked to a light chain constant region comprising the sequence of SEQ ID NO: 3644;
(G)(i) the first polypeptide chain comprises a VH comprising the sequence of SEQ ID NO: 1196, wherein the VH is linked to a heavy chain constant region comprising the sequence of SEQ ID NO: 3649 or SEQ ID NO: 3452, wherein the heavy chain constant region comprises the first Fc region comprising the sequence of SEQ ID NO: 3447 or SEQ ID NO: 3453,
(ii) the second polypeptide chain comprises a cytokine molecule, wherein the cytokine molecule is linked to the second Fc region comprising the sequence of SEQ ID NO: 3648 or SEQ ID NO: 3454, wherein the cytokine molecule comprises IL-2 or a functional variant thereof comprising the sequence of SEQ ID NO: 2270, and
(iii) the third polypeptide chain comprises a VL comprising the sequence of SEQ ID NO: 1195, wherein the VL is linked to a light chain constant region comprising the sequence of SEQ ID NO: 3644;
(H)(i) the first polypeptide chain comprises a VH comprising the sequence of SEQ ID NO: 3455, wherein the VH is linked to a heavy chain constant region comprising the sequence of SEQ ID NO: 3649 or SEQ ID NO: 3452, wherein the heavy chain constant region comprises the first Fc region comprising the sequence of SEQ ID NO: 3447 or SEQ ID NO: 3453,
(ii) the second polypeptide chain comprises a cytokine molecule, wherein the cytokine molecule is linked to the second Fc region comprising the sequence of SEQ ID NO: 3648 or SEQ ID NO: 3454, wherein the cytokine molecule comprises IL-2 or a functional variant thereof comprising the sequence of SEQ ID NO: 2270, and
(iii) the third polypeptide chain comprises a VL comprising the sequence of SEQ ID NO: 3456, wherein the VL is linked to a light chain constant region comprising the sequence of SEQ ID NO: 3644;
(I)(i) the first polypeptide chain comprises a VH comprising the sequence of SEQ ID NO: 3457, wherein the VH is linked to a heavy chain constant region comprising the sequence of SEQ ID NO: 3459, wherein the heavy chain constant region comprises the first Fc region comprising the sequence of SEQ ID NO: 3460,
(ii) the second polypeptide chain comprises a cytokine molecule, wherein the cytokine molecule is linked to the second Fc region comprising the sequence of SEQ ID NO: 3462, wherein the cytokine molecule comprises IL-2 or a functional variant thereof comprising the sequence of SEQ ID NO: 2270 or the sequence of SEQ ID NO: 2191, and
(iii) the third polypeptide chain comprises a VL comprising the sequence of SEQ ID NO: 3458, wherein the VL is linked to a light chain constant region comprising the sequence of SEQ ID NO: 3461;
(J)(i) the first polypeptide chain comprises a VH comprising the sequence of SEQ ID NO: 3463, wherein the VH is linked to a heavy chain constant region comprising the sequence of SEQ ID NO: 3459, wherein the heavy chain constant region comprises the first Fc region comprising the sequence of SEQ ID NO: 3460,
(ii) the second polypeptide chain comprises a cytokine molecule, wherein the cytokine molecule is linked to the second Fc region comprising the sequence of SEQ ID NO: 3462, wherein the cytokine molecule comprises IL-2 or a functional variant thereof comprising the sequence of SEQ ID NO: 2270 or the sequence of SEQ ID NO: 2191, and
(iii) the third polypeptide chain comprises a VL comprising the sequence of SEQ ID NO: 3464, wherein the VL is linked to a light chain constant region comprising the sequence of SEQ ID NO: 3461;
(K)(i) the first polypeptide chain comprises a VH comprising the sequence of SEQ ID NO: 3465, wherein the VH is linked to a heavy chain constant region comprising the sequence of SEQ ID NO: 3467, wherein the heavy chain constant region comprises the first Fc region comprising the sequence of SEQ ID NO: 3468,
(ii) the second polypeptide chain comprises a cytokine molecule, wherein the cytokine molecule is linked to the second Fc region comprising the sequence of SEQ ID NO: 3469, wherein the cytokine molecule comprises IL-2 or a functional variant thereof comprising the sequence of SEQ ID NO: 2270 or the sequence of SEQ ID NO: 2191, and
(iii) the third polypeptide chain comprises a VL comprising the sequence of SEQ ID NO: 3466, wherein the VL is linked to a light chain constant region comprising the sequence of SEQ ID NO: 3461;
(L)(i) the first polypeptide chain comprises a VH comprising the sequence of SEQ ID NO: 3470, wherein the VH is linked to a heavy chain constant region comprising the sequence of SEQ ID NO: 3649 or SEQ ID NO: 3452, wherein the heavy chain constant region comprises the first Fc region comprising the sequence of SEQ ID NO: 3447 or SEQ ID NO: 3453,
(ii) the second polypeptide chain comprises a cytokine molecule, wherein the cytokine molecule is linked to the second Fc region comprising the sequence of SEQ ID NO: 3648 or SEQ ID NO: 3454, wherein the cytokine molecule comprises IL-2 or a functional variant thereof comprising the sequence of SEQ ID NO: 2270, and
(iii) the third polypeptide chain comprises a VL comprising the sequence of SEQ ID NO: 3471, wherein the VL is linked to a light chain constant region comprising the sequence of SEQ ID NO: 3644;
(M)(i) the first polypeptide chain comprises a VH comprising the sequence of SEQ ID NO: 1346, wherein the VH is linked to a heavy chain constant region comprising the sequence of SEQ ID NO: 3452 or the sequence of SEQ ID NO: 3504, wherein the heavy chain constant region comprises the first Fc region comprising the sequence of SEQ ID NO: 3453 or the sequence of SEQ ID NO: 3505,
(ii) the second polypeptide chain comprises a cytokine molecule, wherein the cytokine molecule is linked to the second Fc region comprising the sequence of SEQ ID NO: 3648 or SEQ ID NO: 3454, wherein the cytokine molecule comprises IL-15 or a functional variant thereof comprising the sequence of SEQ ID NO: 2170, wherein the IL-15 or a functional variant thereof is covalently linked to an IL15Ralpha dimerizing domain comprising the sequence of the SEQ ID NO: 3472, and
(iii) the third polypeptide chain comprises a VL comprising the sequence of SEQ ID NO: 3475, wherein the VL is linked to a light chain constant region comprising the sequence of SEQ ID NO: 3644; or
(N)(i) the first polypeptide chain comprises a VH comprising the sequence of SEQ ID NO: 1346, wherein the VH is linked to a heavy chain constant region comprising the sequence of SEQ ID NO: 3452 or the sequence of SEQ ID NO: 3504, wherein the heavy chain constant region comprises the first Fc region comprising the sequence of SEQ ID NO: 3453 or the sequence of SEQ ID NO: 3505,
(ii) the second polypeptide chain comprises a cytokine molecule, wherein the cytokine molecule is linked to the second Fc region comprising the sequence of SEQ ID NO: 3648 or SEQ ID NO: 3454, wherein the cytokine molecule comprises IL-15 or a functional variant thereof comprising the sequence of SEQ ID NO: 2170, wherein the IL-15 or a functional variant thereof is covalently linked to an IL15Ralpha dimerizing domain comprising the sequence of the SEQ ID NO: 3472, and
(iii) the third polypeptide chain comprises a VL comprising the sequence of SEQ ID NO: 3476, wherein the VL is linked to a light chain constant region comprising the sequence of SEQ ID NO: 3644.

600. The method of claim 1, wherein the first agent and/or the second agent comprises a first polypeptide chain, a second polypeptide chain, and a third polypeptide chain, wherein the first polypeptide chain, the second polypeptide chain, and the third polypeptide chain are non-contiguous, and
wherein:
(A)(a)(i) the first polypeptide chain comprises a sequence having at least 75% sequence identity to the sequence of SEQ ID NO: 3348 or the sequence of SEQ ID NO: 3502;
(ii) the second polypeptide chain comprises a sequence having at least 75% sequence identity to the sequence of SEQ ID NO: 3340, the sequence of SEQ ID NO: 3503, or the sequence of SEQ ID NO: 3350; and
(iii) the third polypeptide chain comprises a sequence having at least 75% sequence identity to the sequence of SEQ ID NO: 3349; or
(b)(i) the first polypeptide chain comprises the sequence of SEQ ID NO: 3348 or the sequence of SEQ ID NO: 3502;
(ii) the second polypeptide chain comprises the sequence of SEQ ID NO: 3340, the sequence of SEQ ID NO: 3503, or the sequence of SEQ ID NO: 3350; and
(iii) the third polypeptide chain comprises the sequence of SEQ ID NO: 3349;
(B)(a)(i) the first polypeptide chain comprises a sequence having at least 75% sequence identity to the sequence of SEQ ID NO: 3338;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3339; or
(b)(i) the first polypeptide chain comprises the sequence of SEQ ID NO: 3338;
(ii) the second polypeptide chain comprises the sequence of SEQ ID NO: 3340 or the sequence of SEQ ID NO: 3503; and
(iii) the third polypeptide chain comprises the sequence of SEQ ID NO: 3339;
(C)(a)(i) the first polypeptide chain comprises a sequence having at least 75% sequence identity to the sequence of SEQ ID NO: 3318;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3319; or
(b)(i) the first polypeptide chain comprises the sequence of SEQ ID NO: 3318;
(ii) the second polypeptide chain comprises the sequence of SEQ ID NO: 3340 or the sequence of SEQ ID NO: 3503; and
(iii) the third polypeptide chain comprises the sequence of SEQ ID NO: 3319;
(D)(a)(i) the first polypeptide chain comprises a sequence having at least 75% sequence identity to the sequence of SEQ ID NO: 3328;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3329; or
(b)(i) the first polypeptide chain comprises the sequence of SEQ ID NO: 3328;
(ii) the second polypeptide chain comprises the sequence of SEQ ID NO: 3340 or the sequence of SEQ ID NO: 3503; and
(iii) the third polypeptide chain comprises the sequence of SEQ ID NO: 3329;
(E)(a)(i) the first polypeptide chain comprises a sequence having at least 75% sequence identity to the sequence of SEQ ID NO: 3328;
(ii) the second polypeptide chain comprises a sequence having at least 75% sequence identity to the sequence of SEQ ID NO: 3350 or the sequence of SEQ ID NO: 3500; and
(iii) the third polypeptide chain comprises a sequence having at least 75% sequence identity to the sequence of SEQ ID NO: 3329; or
(b)(i) the first polypeptide chain comprises the sequence of SEQ ID NO: 3328;
(ii) the second polypeptide chain comprises the sequence of SEQ ID NO: 3350 or the sequence of SEQ ID NO: 3500; and
(iii) the third polypeptide chain comprises the sequence of SEQ ID NO: 3329;
(F)(a)(i) the first polypeptide chain comprises a sequence having at least 75% sequence identity to the sequence of SEQ ID NO: 3358;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3359; or
(b)(i) the first polypeptide chain comprises the sequence of SEQ ID NO: 3358;
(ii) the second polypeptide chain comprises the sequence of SEQ ID NO: 3340 or the sequence of SEQ ID NO: 3503; and
(iii) the third polypeptide chain comprises the sequence of SEQ ID NO: 3359;
(G)(a)(i) the first polypeptide chain comprises a sequence having at least 75% sequence identity to the sequence of SEQ ID NO: 3368;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3369; or
(b)(i) the first polypeptide chain comprises the sequence of SEQ ID NO: 3368;
(ii) the second polypeptide chain comprises the sequence of SEQ ID NO: 3340 or the sequence of SEQ ID NO: 3503; and
(iii) the third polypeptide chain comprises the sequence of SEQ ID NO: 3369;
(H)(a)(i) the first polypeptide chain comprises a sequence having at least 75% sequence identity to the sequence of SEQ ID NO: 3378;
(ii) the second polypeptide chain comprises a sequence having at least 75% sequence identity to the sequence of SEQ ID NO: 3380; and
(iii) the third polypeptide chain comprises a sequence having at least 75% sequence identity to the sequence of SEQ ID NO: 3379; or
(b)(i) the first polypeptide chain comprises the sequence of SEQ ID NO: 3378;
(ii) the second polypeptide chain comprises the sequence of SEQ ID NO: 3380; and
(iii) the third polypeptide chain comprises the sequence of SEQ ID NO: 3379;
(I)(a)(i) the first polypeptide chain comprises a sequence having at least 75% sequence identity to the sequence of SEQ ID NO: 3388;
(ii) the second polypeptide chain comprises a sequence having at least 75% sequence identity to the sequence of SEQ ID NO: 3390; and
(iii) the third polypeptide chain comprises a sequence having at least 75% sequence identity to the sequence of SEQ ID NO: 3389; or
(b)(i) the first polypeptide chain comprises the sequence of SEQ ID NO: 3388;
(ii) the second polypeptide chain comprises the sequence of SEQ ID NO: 3390; and
(iii) the third polypeptide chain comprises the sequence of SEQ ID NO: 3389;
(J)(a)(i) the first polypeptide chain comprises a sequence having at least 75% sequence identity to the sequence of SEQ ID NO: 3398;
(ii) the second polypeptide chain comprises a sequence having at least 75% sequence identity to the sequence of SEQ ID NO: 3400; and
(iii) the third polypeptide chain comprises a sequence having at least 75% sequence identity to the sequence of SEQ ID NO: 3399; or
(b)(i) the first polypeptide chain comprises the sequence of SEQ ID NO: 3398;
(ii) the second polypeptide chain comprises the sequence of SEQ ID NO: 3400; and
(iii) the third polypeptide chain comprises the sequence of SEQ ID NO: 3399;
(K)(a)(i) the first polypeptide chain comprises a sequence having at least 75% sequence identity to the sequence of SEQ ID NO: 3408;
(ii) the second polypeptide chain comprises a sequence having at least 75% sequence identity to the sequence of SEQ ID NO: 3340 or the sequence of SEQ ID NO: 3503; and
(iii) the third polypeptide chain comprises a sequence having at least 75% sequence identity to the sequence of SEQ ID NO: 3409; or
(b)(i) the first polypeptide chain comprises the sequence of SEQ ID NO: 3408;
(ii) the second polypeptide chain comprises the sequence of SEQ ID NO: 3340 or the sequence of SEQ ID NO: 3503; and
(iii) the third polypeptide chain comprises the sequence of SEQ ID NO: 3409;
(L)(a)(i) the first polypeptide chain comprises a sequence having at least 75% sequence identity to the sequence of SEQ ID NO: 3348 or the sequence of SEQ ID NO: 3502;
(ii) the second polypeptide chain comprises a sequence having at least 75% sequence identity to the sequence of SEQ ID NO: 3420; and
(iii) the third polypeptide chain comprises a sequence having at least 75% sequence identity to the sequence of SEQ ID NO: 3349; or
(b)(i) the first polypeptide chain comprises the sequence of SEQ ID NO: 3348 or the sequence of SEQ ID NO: 3502;
(ii) the second polypeptide chain comprises the sequence of SEQ ID NO: 3420; and
(iii) the third polypeptide chain comprises the sequence of SEQ ID NO: 3349;
(M)(a)(i) the first polypeptide chain comprises a sequence having at least 75% sequence identity to the sequence of SEQ ID NO: 3348 or the sequence of SEQ ID NO: 3502;
(ii) the second polypeptide chain comprises a sequence having at least 75% sequence identity to the sequence of SEQ ID NO: 3420; and
(iii) the third polypeptide chain comprises a sequence having at least 75% sequence identity to the sequence of SEQ ID NO: 3428; or
(b)(i) the first polypeptide chain comprises the sequence of SEQ ID NO: 3348 or the sequence of SEQ ID NO: 3502;
(ii) the second polypeptide chain comprises the sequence of SEQ ID NO: 3420; and
(iii) the third polypeptide chain comprises the sequence of SEQ ID NO: 3428; or
(N)(a)(i) the first polypeptide chain comprises a sequence having at least 75% sequence identity to the sequence of SEQ ID NO: 3348 or the sequence of SEQ ID NO: 3502;
(ii) the second polypeptide chain comprises a sequence having at least 75% sequence identity to the sequence of SEQ ID NO: 3420; and
(iii) the third polypeptide chain comprises a sequence having at least 75% sequence identity to the sequence of SEQ ID NO: 3437; or
(b)(i) the first polypeptide chain comprises the sequence of SEQ ID NO: 3348 or the sequence of SEQ ID NO: 3502;
(ii) the second polypeptide chain comprises the sequence of SEQ ID NO: 3420; and
(iii) the third polypeptide chain comprises the sequence of SEQ ID NO: 3437.

601. The method of claim 1, wherein the disease or condition is cancer.


SEQUENCE LISTING (3702 sequences)

SEQ ID NO: 1 - protein, 119 aa, synthetic construct
QVQLQQSGPELVKPGTSVKISCKASGYSFTTYYIHWVKQRPGQGLEWIGWFFPGSGNIKYNEKFKGKATLTADTSSSTAYMQLSSLTSEESAVYFCAGSYYSYDVLDYWGHGTTLTVSS

SEQ ID NO: 2 - protein, 107 aa, synthetic construct
DILMTQSQKFMSTSLGDRVSVSCKASQNVGINVVWHQQKPGQSPKALIYSSSHRYSGVPDRFTGSGSGTDFTLTINNVQSEDLAEYFCQQFKSYPLTFGAGTKLELK

SEQ ID NO: 3 - protein, 10 aa, synthetic construct
GYSFTTYYIH

SEQ ID NO: 4 - protein, 17 aa, synthetic construct
WFFPGSGNIKYNEKFKG

SEQ ID NO: 5 - protein, 10 aa, synthetic construct
SYYSYDVLDY

SEQ ID NO: 6 - protein, 11 aa, synthetic construct
KASQNVGINVV

SEQ ID NO: 7 - protein, 7 aa, synthetic construct
SSSHRYS

SEQ ID NO: 8 - protein, 9 aa, synthetic construct
QQFKSYPLT

SEQ ID NO: 9 - protein, 119 aa, synthetic construct
QVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSS

SEQ ID NO: 10 - protein, 107 aa, synthetic construct
DIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 11 - protein, 107 aa, synthetic construct
DIQMTQSPSSLSASVGDRVTITCKASQNVGINVVWHQQKPGKVPKALIYSSSHRYSGVPSRFSGSGSGTDFTLTISSLQPEDVATYFCQQFKSYPLTFGQGTKLEIK

SEQ ID NO: 12 - DNA, 357 bp, synthetic construct
  source 1..357: mol_type = other DNA
caggtgcagctggttcagtctggcgccgaagtgaagaaacctggctcctccgtgaaggtgtcctgcaaggcttccggctactccttcaccacctactacatccactgggtccgacaggcccctggacaaggattggaatggatgggctggttcttccccggctccggcaacatcaagtacaacgagaagttcaagggccgcgtgaccatcaccgccgacacctctacctctaccgcctacatggaactgtccagcctgagatctgaggacaccgccgtgtactactgcgccggctcctactactcttacgacgtgctggattactggggccagggcaccacagtgacagtgtcctct

SEQ ID NO: 13 - DNA, 321 bp, synthetic construct
  source 1..321: mol_type = other DNA
gacatccagatgacccagtctccatccttcctgtccgcctctgtgggcgacagagtgaccatcacatgcaaggcctctcagaacgtgggcatcaacgtcgtgtggcaccagcagaagcctggcaaggctcctaaggctctgatctactcctccagccaccggtactctggcgtgccctctagattttccggctctggctctggcaccgagtttaccctgacaatctccagcctgcagcctgaggacttcgccacctacttttgccagcagttcaagagctaccctctgacctttggccagggcaccaagctggaaatcaag

SEQ ID NO: 14 - DNA, 321 bp, synthetic construct
  source 1..321: mol_type = other DNA
gacatccagatgacccagtctccatcctctctgtccgcctctgtgggcgacagagtgaccatcacatgcaaggcctctcagaacgtgggcatcaacgtcgtgtggcaccagcagaaacctggcaaggtgcccaaggctctgatctactcctccagccacagatactccggcgtgccctctagattctccggctctggctctggcaccgactttaccctgacaatctccagcctgcagcctgaggacgtggccacctacttttgccagcagttcaagagctaccctctgacctttggccagggcaccaagctggaaatcaag

SEQ ID NO: 15 - protein, 117 aa, synthetic construct
DVQLVESGGGLVQPGGSRKLSCAASGFTFSNFGMHWVRQAPDKGLEWVAYISSGSSTIYYADTLKGRFTISRDNPKNTLFLQMTSLRSEDTAMYYCARRGEGAMDYWGQGTSVTVSS

SEQ ID NO: 16 - protein, 106 aa, synthetic construct
ENVLTQSPAIMSASLGEKVTMSCRASSSVNYIYWYQQKSDASPKLWIYYTSNLAPGVPTRFSGSGSGNSYSLTISSMEGEDAATYYCQQFTSSPFTFGSGTKLEIK

SEQ ID NO: 17 - protein, 10 aa, synthetic construct
GFTFSNFGMH

SEQ ID NO: 18 - protein, 17 aa, synthetic construct
YISSGSSTIYYADTLKG

SEQ ID NO: 19 - protein, 8 aa, synthetic construct
RGEGAMDY

SEQ ID NO: 20 - protein, 10 aa, synthetic construct
RASSSVNYIY

SEQ ID NO: 21 - protein, 7 aa, synthetic construct
YTSNLAP

SEQ ID NO: 22 - protein, 9 aa, synthetic construct
QQFTSSPFT

SEQ ID NO: 23 - protein, 117 aa, synthetic construct
QVQLVESGGGVVQPGRSLRLSCAASGFTFSNFGMHWVRQAPGKGLEWVAYISSGSSTIYYADTLKGRFTISRDNSKNTLYLQMNSLRAEDTAVYYCARRGEGAMDYWGQGTTVTVSS

SEQ ID NO: 24 - skipped

SEQ ID NO: 25 - protein, 117 aa, synthetic construct
EVQLVESGGGLVQPGGSLRLSCAASGFTFSNFGMHWVRQAPGKGLEWVSYISSGSSTIYYADTLKGRFTISRDNSKNTLYLQMNSLRAEDTAVYYCARRGEGAMDYWGQGTTVTVSS

SEQ ID NO: 26 - protein, 106 aa, synthetic construct
DNQLTQSPSFLSASVGDRVTITCRASSSVNYIYWYQQKPGKAPKLLIYYTSNLAPGVPSRFSGSGSGNEYTLTISSLQPEDFATYYCQQFTSSPFTFGQGTKLEIK

SEQ ID NO: 27 - protein, 106 aa, synthetic construct
DNQLTQSPSSLSASVGDRVTITCRASSSVNYIYWYQQKPGKAPKLLIYYTSNLAPGVPSRFSGSGSGNDYTLTISSLQPEDFATYYCQQFTSSPFTFGQGTKLEIK

SEQ ID NO: 28 - protein, 106 aa, synthetic construct
ENVLTQSPATLSVSPGERATLSCRASSSVNYIYWYQQKPGQAPRLLIYYTSNLAPGIPARFSGSGSGNEYTLTISSLQSEDFAVYYCQQFTSSPFTFGQGTKLEIK

SEQ ID NO: 29 - protein, 105 aa, synthetic construct
QNVLTQPPSASGTPGQRVTISCRASSSVNYIYWYQQLPGTAPKLLIYYTSNLAPGVPDRFSGSGSGNSYSLAISGLRSEDEADYYCQQFTSSPFTFGTGTKVTVL

SEQ ID NO: 30 - protein, 105 aa, synthetic construct
SNELTQPPSVSVSPGQTARITCRASSSVNYIYWYQQKSGQAPVLVIYYTSNLAPGIPERFSGSGSGNMYTLTISGAQVEDEADYYCQQFTSSPFTFGTGTKVTVL

SEQ ID NO: 31 - DNA, 351 bp, synthetic construct
  source 1..351: mol_type = other DNA
gaggtgcagctggttgaatctggcggaggattggttcagcctggcggctctctgagactgtcttgtgccgcttctggcttcaccttctccaacttcggcatgcactgggtccgacaggcccctggaaaaggactggaatgggtgtcctacatctcctccggctcctccaccatctactacgctgacaccctgaagggcagattcaccatctctcgggacaacgccaagaactccctgtacctgcagatgaacagcctgagagccgaggacaccgccgtgtactactgtgctagaagaggcgagggcgccatggattattggggccagggaaccacagtgaccgtgtctagc

SEQ ID NO: 32 - DNA, 351 bp, synthetic construct
  source 1..351: mol_type = other DNA
gaggtgcagctggttgaatctggcggaggattggttcagcctggcggctctctgagactgtcttgtgccgcttctggcttcaccttctccaacttcggcatgcactgggtccgacaggcccctggaaaaggactggaatgggtgtcctacatctcctccggctcctccaccatctactacgctgacaccctgaagggcagattcaccatcagccgggacaactccaagaacaccctgtacctgcagatgaactccctgagagccgaggacaccgccgtgtactactgtgctagaagaggcgagggcgccatggattattggggccagggaaccacagtgaccgtgtctagc

SEQ ID NO: 33 - DNA, 351 bp, synthetic construct
  source 1..351: mol_type = other DNA
caggtgcagctggtggaatctggtggcggagttgtgcagcctggcagatccctgagactgtcttgtgccgcctctggcttcaccttctccaacttcggcatgcactgggtccgacaggcccctggaaaaggattggagtgggtcgcctacatctcctccggctcctccaccatctactacgctgacaccctgaagggcagattcaccatcagccgggacaactccaagaacaccctgtacctgcagatgaactccctgagagccgaggacaccgccgtgtactactgtgctagaagaggcgagggcgccatggattattggggccagggaaccacagtgaccgtgtctagc

SEQ ID NO: 34 - DNA, 318 bp, synthetic construct
  source 1..318: mol_type = other DNA
gataaccagctgacccagtctcctagcttcctgtctgcctctgtgggcgacagagtgacaattacctgccgggcctcctcctccgtgaactacatctactggtatcagcagaagcccggcaaggcccctaagctgctgatctactacacctccaatctggcccctggcgtgccctctagattttccggatctggctccggcaacgagtataccctgacaatctccagcctgcagcctgaggacttcgccacctactactgccagcagttcacctcctctccattcacctttggccagggcaccaagctggaaatcaaa

SEQ ID NO: 35 - DNA, 317 bp, synthetic construct
  source 1..317: mol_type = other DNA
ataaccagctgacccagtctccttccagcctgtctgcttctgtgggcgacagagtgacaattacctgccgggcctcctcctccgtgaactacatctactggtatcagcagaagcccggcaaggcccctaagctgctgatctactacacctccaatctggcccctggcgtgccctctagattttccggatctggctccggcaacgactataccctgacaatctccagcctgcagcctgaggacttcgccacctactactgccagcagttcacctcctctccattcacctttggccagggcaccaagctggaaatcaaa

SEQ ID NO: 36 - DNA, 318 bp, synthetic construct
  source 1..318: mol_type = other DNA
gagaatgtgctgacccagtctcctgccacactgtctgttagccctggcgagagagctaccctgagctgcagagcctcttcctccgtgaactacatctactggtatcagcagaagcccggccaggctcctagactgctgatctactacacctccaatctggcccctggcatccctgccagattttccggatctggctccggcaacgagtataccctgaccatctccagcctgcagtccgaggactttgctgtgtactattgccagcagttcacaagcagccctttcacctttggccagggcaccaagctggaaatcaaa

SEQ ID NO: 37 - DNA, 315 bp, synthetic construct
  source 1..315: mol_type = other DNA
cagaatgtgctgacccaacctccttccgcctctggcacacctggacagagagtgacaatctcctgccgggcctcctcctccgtgaactacatctactggtatcagcagctgcccggcaccgctcctaaactgctgatctactacacctccaatctggcccctggcgtgcccgatagattttccggatctggctccggcaactcctacagcctggctatctctggcctgagatctgaggacgaggccgactactactgccagcagttcacctcctctccattcacctttggcaccggcaccaaagtgacagttctt

SEQ ID NO: 38 - DNA, 315 bp, synthetic construct
  source 1..315: mol_type = other DNA
tctaatgagctgacccagcctccttccgtgtccgtgtctcctggacagaccgccagaattacctgccgggcctcctcctccgtgaactacatctactggtatcagcagaagtccggccaggctcctgtgctcgtgatctactacacctccaatctggcccctggcatccctgagagattctccggatctggctccggcaacatgtacaccctgaccatctctggcgcccaggtggaagatgaggccgactactactgccagcagttcacctcctctccattcacctttggcaccggcaccaaagtgacagttctt

SEQ ID NO: 39 - protein, 107 aa, synthetic construct [sequence omitted]

SEQ ID NO: 40 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 41 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - DNA, 344 bp, synthetic construct
  source 1..344: mol_type = other DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttactc

SEQ ID NO: 44 - protein, 114 aa, synthetic construct
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSY

SEQ ID NO: 45 - protein, 5 aa, synthetic construct
TYYIH

SEQ ID NO: 46 - protein, 17 aa, synthetic construct
WFFPGSGNIKYNEKFKG

SEQ ID NO: 47 - protein, 10 aa, synthetic construct
SYYSYDVLDY

SEQ ID NO: 48 - protein, 7 aa, synthetic construct
GYSFTTY

SEQ ID NO: 49 - protein, 6 aa, synthetic construct
FPGSGN

SEQ ID NO: 50 - protein, 10 aa, synthetic construct
SYYSYDVLDY

SEQ ID NO: 51 - protein, 11 aa, synthetic construct
KASQNVGINVV

SEQ ID NO: 52 - protein, 7 aa, synthetic construct
SSSHRYS

SEQ ID NO: 53 - protein, 9 aa, synthetic construct
QQFKSYPLT

SEQ ID NO: 54 - protein, 11 aa, synthetic construct
KASQNVGINVV

SEQ ID NO: 55 - protein, 7 aa, synthetic construct
SSSHRYS

SEQ ID NO: 56 - protein, 9 aa, synthetic construct
QQFKSYPLT

SEQ ID NO: 57 - protein, 5 aa, synthetic construct
NFGMH

SEQ ID NO: 58 - protein, 17 aa, synthetic construct
YISSGSSTIYYADTLKG

SEQ ID NO: 59 - protein, 8 aa, synthetic construct
RGEGAMDY

SEQ ID NO: 60 - protein, 7 aa, synthetic construct
GFTFSNF

SEQ ID NO: 61 - protein, 6 aa, synthetic construct
SSGSST

SEQ ID NO: 62 - protein, 8 aa, synthetic construct
RGEGAMDY

SEQ ID NO: 63 - protein, 10 aa, synthetic construct
RASSSVNYIY

SEQ ID NO: 64 - protein, 7 aa, synthetic construct
YTSNLAP

SEQ ID NO: 65 - protein, 9 aa, synthetic construct
QQFTSSPFT

SEQ ID NO: 66 - protein, 10 aa, synthetic construct
RASSSVNYIY

SEQ ID NO: 67 - protein, 7 aa, synthetic construct
YTSNLAP

SEQ ID NO: 68 - protein, 9 aa, synthetic construct
QQFTSSPFT

SEQ ID NO: 69 - protein, 592 aa, synthetic construct
METDTLLLWVLLLWVPGSTGQVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLASSLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 70 - protein, 492 aa, synthetic construct
METDTLLLWVLLLWVPGSTGQVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSSASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 71 - protein, 479 aa, synthetic construct
METDTLLLWVLLLWVPGSTGQVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSSASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 72 - protein, 234 aa, synthetic construct
METDTLLLWVLLLWVPGSTGDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 453 aa, synthetic construct
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLASSLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 74 - protein, 353 aa, synthetic construct
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 75 - protein, 340 aa, synthetic construct
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 76 - protein, 159 aa, synthetic construct
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 77-79 - skipped (3 entries)

SEQ ID NO: 80 - protein, 246 aa, synthetic construct
QVQLVQSGAEVKKPGSSVKVSCKASGTDFKLTYIHWVRQAPGQGLEWMGRVSPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDRVAWYQQKPGKAPKALIYSSSHRYKGVPSRFSGSGSGTEFTLTISSLQPEDFATYFCQQFKSYPLTFGQGTKLEIK

SEQ ID NO: 81 - protein, 107 aa, synthetic construct
DIQMTQSPSFLSASVGDRVTITCKASQNVEDRVAWYQQKPGKAPKALIYSSSHRYKGVPSRFSGSGSGTEFTLTISSLQPEDFATYFCQQFKSYPLTFGQGTKLEIK

SEQ ID NO: 82 - protein, 119 aa, synthetic construct
QVQLVQSGAEVKKPGSSVKVSCKASGTDFKLTYIHWVRQAPGQGLEWMGRVSPGSGNTKYNEKFKGRVTITADTSTSTAYMELSSLRSEDTAVYYCAGSYYSYDVLDYWGQGTTVTVSS

SEQ ID NO: 83 - protein, 246 aa, synthetic construct
QVQLVQSGAEVKKPGSSVKVSCKASGTDFDKIYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDRVAWYQQKPGKAPKALIYSSSHRYKGVPSRFSGSGSGTEFTLTISSLQPEDFATYFCQQFKSYPLTFGQGTKLEIK

SEQ ID NO: 84 - protein, 107 aa, synthetic construct; identical to SEQ ID NO: 81

SEQ ID NO: 85 - protein, 119 aa, synthetic construct
QVQLVQSGAEVKKPGSSVKVSCKASGTDFDKIYIHWVRQAPGQGLEWMGRISAGSGNVKYNEKFKGRVTITADTSTSTAYMELSSLRSEDTAVYYCAGSYYSYDVLDYWGQGTTVTVSS

SEQ ID NO: 86 - protein, 246 aa, synthetic construct
QVQLVQSGAEVKKPGSSVKVSCKASGHDFRDFYIHWVRQAPGQGLEWMGRVYPGSGSYRYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 87 - protein, 107 aa, synthetic construct
DIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 88 - protein, 119 aa, synthetic construct
QVQLVQSGAEVKKPGSSVKVSCKASGHDFRDFYIHWVRQAPGQGLEWMGRVYPGSGSYRYNEKFKGRVTITADTSTSTAYMELSSLRSEDTAVYYCAGSYYSYDVLDYWGQGTTVTVSS

SEQ ID NO: 89 - protein, 246 aa, synthetic construct
QVQLVQSGAEVKKPGSSVKVSCKASGHDFKLTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNRVAWYQQKPGKAPKALIYSSSHRYKGVPSRFSGSGSGTEFTLTISSLQPEDFATYFCQQFKSYPLTFGQGTKLEIK

SEQ ID NO: 90 - protein, 107 aa, synthetic construct
DIQMTQSPSFLSASVGDRVTITCKASQNVDNRVAWYQQKPGKAPKALIYSSSHRYKGVPSRFSGSGSGTEFTLTISSLQPEDFATYFCQQFKSYPLTFGQGTKLEIK

SEQ ID NO: 91 - protein, 119 aa, synthetic construct
QVQLVQSGAEVKKPGSSVKVSCKASGHDFKLTYIHWVRQAPGQGLEWMGRISAGSGNVKYNEKFKGRVTITADTSTSTAYMELSSLRSEDTAVYYCAGSYYSYDVLDYWGQGTTVTVSS

SEQ ID NO: 92 - protein, 246 aa, synthetic construct
QVQLVQSGAEVKKPGSSVKVSCKASGTDFKLTYIHWVRQAPGQGLEWMGRIFPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVENKVAWHQQKPGKAPKALIYSSSHRYKGVPSRFSGSGSGTEFTLTISSLQPEDFATYFCQQFKSYPLTFGQGTKLEIK

SEQ ID NO: 93 - protein, 107 aa, synthetic construct
DIQMTQSPSFLSASVGDRVTITCKASQNVENKVAWHQQKPGKAPKALIYSSSHRYKGVPSRFSGSGSGTEFTLTISSLQPEDFATYFCQQFKSYPLTFGQGTKLEIK

SEQ ID NO: 94 - protein, 119 aa, synthetic construct
QVQLVQSGAEVKKPGSSVKVSCKASGTDFKLTYIHWVRQAPGQGLEWMGRIFPGSGNVKYNEKFKGRVTITADTSTSTAYMELSSLRSEDTAVYYCAGSYYSYDVLDYWGQGTTVTVSS

SEQ ID NO: 95 - protein, 246 aa, synthetic construct
QVQLVQSGAEVKKPGSSVKVSCKASGTDFDKIYIHWVRQAPGQGLEWMGRIFAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 96 - protein, 107 aa, synthetic construct; identical to SEQ ID NO: 87

SEQ ID NO: 97 - protein, 119 aa, synthetic construct
QVQLVQSGAEVKKPGSSVKVSCKASGTDFDKIYIHWVRQAPGQGLEWMGRIFAGSGNTKYNEKFKGRVTITADTSTSTAYMELSSLRSEDTAVYYCAGSYYSYDVLDYWGQGTTVTVSS

SEQ ID NO: 98 - protein, 246 aa, synthetic construct
QVQLVQSGAEVKKPGSSVKVSCKASGHDFDKFYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGNRVAWYQQKPGKAPKALIYSSSHRYSGVPSRFSGSGSGTEFTLTISSLQPEDFATYFCQQFKSYPLTFGQGTKLEIK

SEQ ID NO: 99 - protein, 107 aa, synthetic construct
DIQMTQSPSFLSASVGDRVTITCKASQNVGNRVAWYQQKPGKAPKALIYSSSHRYSGVPSRFSGSGSGTEFTLTISSLQPEDFATYFCQQFKSYPLTFGQGTKLEIK

SEQ ID NO: 100 - protein, 119 aa, synthetic construct
QVQLVQSGAEVKKPGSSVKVSCKASGHDFDKFYIHWVRQAPGQGLEWMGRVSAGSGNVKYNEKFKGRVTITADTSTSTAYMELSSLRSEDTAVYYCAGSYYSYDVLDYWGQGTTVTVSS

SEQ ID NO: 101 - protein, 246 aa, synthetic construct
QVQLVQSGAEVKKPGSSVKVSCKASGHDFDKFYIHWVRQAPGQGLEWMGRIFAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWYQQKPGKAPKALIYSSSHRYKGVPSRFSGSGSGTEFTLTISSLQPEDFATYFCQQFKSYPLTFGQGTKLEIK

SEQ ID NO: 102 - protein, 107 aa, synthetic construct
DIQMTQSPSFLSASVGDRVTITCKASQNVGDRVAWYQQKPGKAPKALIYSSSHRYKGVPSRFSGSGSGTEFTLTISSLQPEDFATYFCQQFKSYPLTFGQGTKLEIK

SEQ ID NO: 103 - protein, 119 aa, synthetic construct
QVQLVQSGAEVKKPGSSVKVSCKASGHDFDKFYIHWVRQAPGQGLEWMGRIFAGSGNVKYNEKFKGRVTITADTSTSTAYMELSSLRSEDTAVYYCAGSYYSYDVLDYWGQGTTVTVSS

SEQ ID NO: 104 - protein, 246 aa, synthetic construct
QVQLVQSGAEVKKPGSSVKVSCKASGTDFKLTYIHWVRQAPGQGLEWMGRVSPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWYQQKPGKAPKALIYSSSHRYKGVPSRFSGSGSGTEFTLTISSLQPEDFATYFCQQFKSYPLTFGQGTKLEIK

SEQ ID NO: 105 - protein, 107 aa, synthetic construct; identical to SEQ ID NO: 102

SEQ ID NO: 106 - protein, 119 aa, synthetic construct
QVQLVQSGAEVKKPGSSVKVSCKASGTDFKLTYIHWVRQAPGQGLEWMGRVSPGSGNVKYNEKFKGRVTITADTSTSTAYMELSSLRSEDTAVYYCAGSYYSYDVLDYWGQGTTVTVSS

SEQ ID NO: 107 - protein, 246 aa, synthetic construct
QVQLVQSGAEVKKPGSSVKVSCKASGTDFDKIYIHWVRQAPGQGLEWMGRVSAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGNRVAWYQQKPGKAPKALIYSSSHRYKGVPSRFSGSGSGTEFTLTISSLQPEDFATYFCQQFKSYPLTFGQGTKLEIK

SEQ ID NO: 108 - protein, 107 aa, synthetic construct
DIQMTQSPSFLSASVGDRVTITCKASQNVGNRVAWYQQKPGKAPKALIYSSSHRYKGVPSRFSGSGSGTEFTLTISSLQPEDFATYFCQQFKSYPLTFGQGTKLEIK

SEQ ID NO: 109 - protein, 119 aa, synthetic construct
QVQLVQSGAEVKKPGSSVKVSCKASGTDFDKIYIHWVRQAPGQGLEWMGRVSAGSGNTKYNEKFKGRVTITADTSTSTAYMELSSLRSEDTAVYYCAGSYYSYDVLDYWGQGTTVTVSS

SEQ ID NO: 110 - protein, 246 aa, synthetic construct
QVQLVQSGAEVKKPGSSVKVSCKASGTDFKLTYIHWVRQAPGQGLEWMGRIFPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNRVAWYQQKPGKAPKALIYSSSHRYKGVPSRFSGSGSGTEFTLTISSLQPEDFATYFCQQFKSYPLTFGQGTKLEIK

SEQ ID NO: 111 - protein, 107 aa, synthetic construct; identical to SEQ ID NO: 90

SEQ ID NO: 112 - protein, 119 aa, synthetic construct; identical to SEQ ID NO: 94

SEQ ID NO: 113 - protein, 246 aa, synthetic construct
QVQLVQSGAEVKKPGSSVKVSCKASGTDFDKIYIHWVRQAPGQGLEWMGRISAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 114 - protein, 107 aa, synthetic construct; identical to SEQ ID NO: 87

SEQ ID NO: 115 - protein, 119 aa, synthetic construct
QVQLVQSGAEVKKPGSSVKVSCKASGTDFDKIYIHWVRQAPGQGLEWMGRISAGSGNTKYNEKFKGRVTITADTSTSTAYMELSSLRSEDTAVYYCAGSYYSYDVLDYWGQGTTVTVSS

SEQ ID NO: 116 - protein, 246 aa, synthetic construct
QVQLVQSGAEVKKPGSSVKVSCKASGTDFRLTYIHWVRQAPGQGLEWMGRVSPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNKVAWHQQKPGKAPKALIYSSSHRYKGVPSRFSGSGSGTEFTLTISSLQPEDFATYFCQQFKSYPLTFGQGTKLEIK

SEQ ID NO: 117 - protein, 107 aa, synthetic construct
DIQMTQSPSFLSASVGDRVTITCKASQNVDNKVAWHQQKPGKAPKALIYSSSHRYKGVPSRFSGSGSGTEFTLTISSLQPEDFATYFCQQFKSYPLTFGQGTKLEIK

SEQ ID NO: 118 - protein, 119 aa, synthetic construct
QVQLVQSGAEVKKPGSSVKVSCKASGTDFRLTYIHWVRQAPGQGLEWMGRVSPGSGNTKYNEKFKGRVTITADTSTSTAYMELSSLRSEDTAVYYCAGSYYSYDVLDYWGQGTTVTVSS

SEQ ID NO: 119 - protein, 246 aa, synthetic construct
QVQLVQSGAEVKKPGSSVKVSCKASGGTFRLTYIHWVRQAPGQGLEWMGRVYPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 120 - protein, 107 aa, synthetic construct; identical to SEQ ID NO: 87

SEQ ID NO: 121 - protein, 119 aa, synthetic construct
QVQLVQSGAEVKKPGSSVKVSCKASGGTFRLTYIHWVRQAPGQGLEWMGRVYPGSGNTKYNEKFKGRVTITADTSTSTAYMELSSLRSEDTAVYYCAGSYYSYDVLDYWGQGTTVTVSS

SEQ ID NO: 122 - protein, 246 aa, synthetic construct
QVQLVQSGAEVKKPGSSVKVSCKASGTDFRLTYIHWVRQAPGQGLEWMGRIFPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 123 - protein, 107 aa, synthetic construct; identical to SEQ ID NO: 87

SEQ ID NO: 124 - protein, 119 aa, synthetic construct
QVQLVQSGAEVKKPGSSVKVSCKASGTDFRLTYIHWVRQAPGQGLEWMGRIFPGSGNTKYNEKFKGRVTITADTSTSTAYMELSSLRSEDTAVYYCAGSYYSYDVLDYWGQGTTVTVSS

SEQ ID NO: 125 - protein, 246 aa, synthetic construct
QVQLVQSGAEVKKPGSSVKVSCKASGTDFKLTYIHWVRQAPGQGLEWMGRIFPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDRVAWYQQKPGKAPKALIYSSSHRYKGVPSRFSGSGSGTEFTLTISSLQPEDFATYFCQQFKSYPLTFGQGTKLEIK

SEQ ID NO: 126 - protein, 107 aa, synthetic construct; identical to SEQ ID NO: 81

SEQ ID NO: 127 - protein, 119 aa, synthetic construct; identical to SEQ ID NO: 94

SEQ ID NO: 128 - protein, 246 aa, synthetic construct
QVQLVQSGAEVKKPGSSVKVSCKASGGTFRLTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWYQQKPGKAPKALIYSSSHRYKGVPSRFSGSGSGTEFTLTISSLQPEDFATYFCQQFKSYPLTFGQGTKLEIK

SEQ ID NO: 129 - protein, 107 aa, synthetic construct; identical to SEQ ID NO: 102

SEQ ID NO: 130 - protein, 119 aa, synthetic construct
QVQLVQSGAEVKKPGSSVKVSCKASGGTFRLTYIHWVRQAPGQGLEWMGRISAGSGNVKYNEKFKGRVTITADTSTSTAYMELSSLRSEDTAVYYCAGSYYSYDVLDYWGQGTTVTVSS

SEQ ID NO: 131 - protein, 246 aa, synthetic construct
QVQLVQSGAEVKKPGSSVKVSCKASGGTFDKTYIHWVRQAPGQGLEWMGRISAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 132 - protein, 107 aa, synthetic construct; identical to SEQ ID NO: 87

SEQ ID NO: 133 - protein, 119 aa, synthetic construct
QVQLVQSGAEVKKPGSSVKVSCKASGGTFDKTYIHWVRQAPGQGLEWMGRISAGSGNTKYNEKFKGRVTITADTSTSTAYMELSSLRSEDTAVYYCAGSYYSYDVLDYWGQGTTVTVSS

SEQ ID NO: 134 - protein, 246 aa, synthetic construct
QVQLVQSGAEVKKPGSSVKVSCKASGHDFDKFYIHWVRQAPGQGLEWMGRISAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 135 - protein, 107 aa, synthetic construct; identical to SEQ ID NO: 87

SEQ ID NO: 136 - protein, 119 aa, synthetic construct
QVQLVQSGAEVKKPGSSVKVSCKASGHDFDKFYIHWVRQAPGQGLEWMGRISAGSGNTKYNEKFKGRVTITADTSTSTAYMELSSLRSEDTAVYYCAGSYYSYDVLDYWGQGTTVTVSS

SEQ ID NO: 137 - protein, 246 aa, synthetic construct
QVQLVQSGAEVKKPGSSVKVSCKASGTDFKLTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNKVAWHQQKPGKAPKALIYSSSHRYKGVPSRFSGSGSGTEFTLTISSLQPEDFATYFCQQFKSYPLTFGQGTKLEIK

SEQ ID NO: 138 - protein, 107 aa, synthetic construct; identical to SEQ ID NO: 117

SEQ ID NO: 139 - protein, 119 aa, synthetic construct
QVQLVQSGAEVKKPGSSVKVSCKASGTDFKLTYIHWVRQAPGQGLEWMGRISAGSGNVKYNEKFKGRVTITADTSTSTAYMELSSLRSEDTAVYYCAGSYYSYDVLDYWGQGTTVTVSS

SEQ ID NO: 140 - protein, 246 aa, synthetic construct
QVQLVQSGAEVKKPGSSVKVSCKASGHDFRLTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVADRVAWYQQKPGKAPKALIYSSSHRYKGVPSRFSGSGSGTEFTLTISSLQPEDFATYFCQQFKSYPLTFGQGTKLEIK

SEQ ID NO: 141 - protein, 107 aa, synthetic construct
DIQMTQSPSFLSASVGDRVTITCKASQNVADRVAWYQQKPGKAPKALIYSSSHRYKGVPSRFSGSGSGTEFTLTISSLQPEDFATYFCQQFKSYPLTFGQGTKLEIK

SEQ ID NO: 142 - protein, 119 aa, synthetic construct
QVQLVQSGAEVKKPGSSVKVSCKASGHDFRLTYIHWVRQAPGQGLEWMGRISAGSGNVKYNEKFKGRVTITADTSTSTAYMELSSLRSEDTAVYYCAGSYYSYDVLDYWGQGTTVTVSS

SEQ ID NO: 143 - protein, 246 aa, synthetic construct
QVQLVQSGAEVKKPGSSVKVSCKASGHDFHLWYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNKVAWHQQKPGKAPKALIYSSSHRYKGVPSRFSGSGSGTEFTLTISSLQPEDFATYFCQQFKSYPLTFGQGTKLEIK

SEQ ID NO: 144 - protein, 107 aa, synthetic construct; identical to SEQ ID NO: 117

SEQ ID NO: 145 - protein, 119 aa, synthetic construct
QVQLVQSGAEVKKPGSSVKVSCKASGHDFHLWYIHWVRQAPGQGLEWMGRVSAGSGNVKYNEKFKGRVTITADTSTSTAYMELSSLRSEDTAVYYCAGSYYSYDVLDYWGQGTTVTVSS

SEQ ID NO: 146 - protein, 246 aa, synthetic construct
QVQLVQSGAEVKKPGSSVKVSCKASGHDFHLWYIHWVRQAPGQGLEWMGRVFAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDKVAWYQQKPGKAPKALIYSSSHRYKGVPSRFSGSGSGTEFTLTISSLQPEDFATYFCQQFKSYPLTFGQGTKLEIK

SEQ ID NO: 147 - protein, 107 aa, synthetic construct
DIQMTQSPSFLSASVGDRVTITCKASQNVEDKVAWYQQKPGKAPKALIYSSSHRYKGVPSRFSGSGSGTEFTLTISSLQPEDFATYFCQQFKSYPLTFGQGTKLEIK

SEQ ID NO: 148 - protein, 119 aa, synthetic construct
QVQLVQSGAEVKKPGSSVKVSCKASGHDFHLWYIHWVRQAPGQGLEWMGRVFAGSGNTKYNEKFKGRVTITADTSTSTAYMELSSLRSEDTAVYYCAGSYYSYDVLDYWGQGTTVTVSS

SEQ ID NO: 149 - protein, 246 aa, synthetic construct
QVQLVQSGAEVKKPGSSVKVSCKASGTDFKLTYIHWVRQAPGQGLEWMGRIFPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 150 - protein, 107 aa, synthetic construct; identical to SEQ ID NO: 87

SEQ ID NO: 151 - protein, 119 aa, synthetic construct
QVQLVQSGAEVKKPGSSVKVSCKASGTDFKLTYIHWVRQAPGQGLEWMGRIFPGSGNTKYNEKFKGRVTITADTSTSTAYMELSSLRSEDTAVYYCAGSYYSYDVLDYWGQGTTVTVSS

SEQ ID NO: 152 - skipped

SEQ ID NO: 153 - protein, 246 aa, synthetic construct
QVQLVQSGAEVKKPGSSVKVSCKASGTDFKLTYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGNRVAWYQQKPGKAPKALIYSSSHRYKGVPSRFSGSGSGTEFTLTISSLQPEDFATYFCQQFKSYPLTFGQGTKLEIK

SEQ ID NO: 154 - protein, 107 aa, synthetic construct; identical to SEQ ID NO: 108

SEQ ID NO: 155 - protein, 119 aa, synthetic construct
QVQLVQSGAEVKKPGSSVKVSCKASGTDFKLTYIHWVRQAPGQGLEWMGRVSAGSGNVKYNEKFKGRVTITADTSTSTAYMELSSLRSEDTAVYYCAGSYYSYDVLDYWGQGTTVTVSS

SEQ ID NO: 156 - protein, 246 aa, synthetic construct
QVQLVQSGAEVKKPGSSVKVSCKASGTDFKLTYIHWVRQAPGQGLEWMGRISPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWYQQKPGKAPKALIYSSSHRYKGVPSRFSGSGSGTEFTLTISSLQPEDFATYFCQQFKSYPLTFGQGTKLEIK

SEQ ID NO: 157 - protein, 107 aa, synthetic construct; identical to SEQ ID NO: 102

SEQ ID NO: 158 - protein, 119 aa, synthetic construct
QVQLVQSGAEVKKPGSSVKVSCKASGTDFKLTYIHWVRQAPGQGLEWMGRISPGSGNTKYNEKFKGRVTITADTSTSTAYMELSSLRSEDTAVYYCAGSYYSYDVLDYWGQGTTVTVSS

SEQ ID NO: 159 - protein, 246 aa, synthetic construct
QVQLVQSGAEVKKPGSSVKVSCKASGHDFHLWYIHWVRQAPGQGLEWMGRISPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWHQQKPGKAPKALIYSSSHRYKGVPSRFSGSGSGTEFTLTISSLQPEDFATYFCQQFKSYPLTFGQGTKLEIK

SEQ ID NO: 160 - protein, 107 aa, synthetic construct
DIQMTQSPSFLSASVGDRVTITCKASQNVGDRVAWHQQKPGKAPKALIYSSSHRYKGVPSRFSGSGSGTEFTLTISSLQPEDFATYFCQQFKSYPLTFGQGTKLEIK

SEQ ID NO: 161 - protein, 119 aa, synthetic construct
QVQLVQSGAEVKKPGSSVKVSCKASGHDFHLWYIHWVRQAPGQGLEWMGRISPGSGNVKYNEKFKGRVTITADTSTSTAYMELSSLRSEDTAVYYCAGSYYSYDVLDYWGQGTTVTVSS

SEQ ID NO: 162 - protein, 246 aa, synthetic construct
QVQLVQSGAEVKKPGSSVKVSCKASGHDFKLTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 163 - protein, 107 aa, synthetic construct; identical to SEQ ID NO: 87

SEQ ID NO: 164 - protein, 119 aa, synthetic construct; identical to SEQ ID NO: 91

SEQ ID NO: 165 - protein, 246 aa, synthetic construct
QVQLVQSGAEVKKPGSSVKVSCKASGTDFHLWYIHWVRQAPGQGLEWMGRVFAGSGSYRYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 166 - protein, 107 aa, synthetic construct; identical to SEQ ID NO: 87

SEQ ID NO: 167 - protein, 119 aa, synthetic construct
QVQLVQSGAEVKKPGSSVKVSCKASGTDFHLWYIHWVRQAPGQGLEWMGRVFAGSGSYRYNEKFKGRVTITADTSTSTAYMELSSLRSEDTAVYYCAGSYYSYDVLDYWGQGTTVTVSS

SEQ ID NO: 168 - protein, 246 aa, synthetic construct
QVQLVQSGAEVKKPGSSVKVSCKASGTDFDKIYIHWVRQAPGQGLEWMGRISAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVADRVAWYQQKPGKAPKALIYSSSHRYKGVPSRFSGSGSGTEFTLTISSLQPEDFATYFCQQFKSYPLTFGQGTKLEIK

SEQ ID NO: 169 - protein, 107 aa, synthetic construct; identical to SEQ ID NO: 141

SEQ ID NO: 170 - protein, 119 aa, synthetic construct; identical to SEQ ID NO: 115

SEQ ID NO: 171 - protein, 246 aa, synthetic construct
QVQLVQSGAEVKKPGSSVKVSCKASGTDFKLTYIHWVRQAPGQGLEWMGRISAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDRVAWYQQKPGKAPKALIYSSSHRYKGVPSRFSGSGSGTEFTLTISSLQPEDFATYFCQQFKSYPLTFGQGTKLEIK

SEQ ID NO: 172 - protein, 107 aa, synthetic construct; identical to SEQ ID NO: 81

SEQ ID NO: 173 - protein, 119 aa, synthetic construct
QVQLVQSGAEVKKPGSSVKVSCKASGTDFKLTYIHWVRQAPGQGLEWMGRISAGSGNTKYNEKFKGRVTITADTSTSTAYMELSSLRSEDTAVYYCAGSYYSYDVLDYWGQGTTVTVSS

SEQ ID NO: 174 - protein, 246 aa, synthetic construct
QVQLVQSGAEVKKPGSSVKVSCKASGTDFRLTYIHWVRQAPGQGLEWMGRISPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGNRVAWYQQKPGKAPKALIYSSSHRYKGVPSRFSGSGSGTEFTLTISSLQPEDFATYFCQQFKSYPLTFGQGTKLEIK

SEQ ID NO: 175 - protein, 107 aa, synthetic construct; identical to SEQ ID NO: 108

SEQ ID NO: 176 - protein, 119 aa, synthetic construct
QVQLVQSGAEVKKPGSSVKVSCKASGTDFRLTYIHWVRQAPGQGLEWMGRISPGSGNTKYNEKFKGRVTITADTSTSTAYMELSSLRSEDTAVYYCAGSYYSYDVLDYWGQGTTVTVSS

SEQ ID NO: 177 - protein, 246 aa, synthetic construct
QVQLVQSGAEVKKPGSSVKVSCKASGHDFDKTYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDRVAWYQQKPGKAPKALIYSSSHRYKGVPSRFSGSGSGTEFTLTISSLQPEDFATYFCQQFKSYPLTFGQGTKLEIK

SEQ ID NO: 178 - protein, 107 aa, synthetic construct; identical to SEQ ID NO: 81

SEQ ID NO: 179 - protein, 119 aa, synthetic construct
QVQLVQSGAEVKKPGSSVKVSCKASGHDFDKTYIHWVRQAPGQGLEWMGRVSAGSGNVKYNEKFKGRVTITADTSTSTAYMELSSLRSEDTAVYYCAGSYYSYDVLDYWGQGTTVTVSS

SEQ ID NO: 180 - protein, 246 aa, synthetic construct
QVQLVQSGAEVKKPGSSVKVSCKASGHDFKLTYIHWVRQAPGQGLEWMGRVSPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDRVAWHQQKPGKAPKALIYSSSHRYKGVPSRFSGSGSGTEFTLTISSLQPEDFATYFCQQFKSYPLTFGQGTKLEIK

SEQ ID NO: 181 - protein, 107 aa, synthetic construct
DIQMTQSPSFLSASVGDRVTITCKASQNVEDRVAWHQQKPGKAPKALIYSSSHRYKGVPSRFSGSGSGTEFTLTISSLQPEDFATYFCQQFKSYPLTFGQGTKLEIK

SEQ ID NO: 182 - protein, 119 aa, synthetic construct
QVQLVQSGAEVKKPGSSVKVSCKASGHDFKLTYIHWVRQAPGQGLEWMGRVSPGSGNTKYNEKFKGRVTITADTSTSTAYMELSSLRSEDTAVYYCAGSYYSYDVLDYWGQGTTVTVSS

SEQ ID NO: 183 - protein, 246 aa, synthetic construct
QVQLVQSGAEVKKPGSSVKVSCKASGHDFDKTYIHWVRQAPGQGLEWMGRIYPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVADRVAWYQQKPGKAPKALIYSSSHRYKGVPSRFSGSGSGTEFTLTISSLQPEDFATYFCQQFKSYPLTFGQGTKLEIK

SEQ ID NO: 184 - protein, 107 aa, synthetic construct; identical to SEQ ID NO: 141

SEQ ID NO: 185 - protein, 119 aa, synthetic construct
QVQLVQSGAEVKKPGSSVKVSCKASGHDFDKTYIHWVRQAPGQGLEWMGRIYPGSGNVKYNEKFKGRVTITADTSTSTAYMELSSLRSEDTAVYYCAGSYYSYDVLDYWGQGTTVTVSS

SEQ ID NO: 186 - protein, 246 aa, synthetic construct
QVQLVQSGAEVKKPGSSVKVSCKASGTDFDKTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 187 - protein, 107 aa, synthetic construct; identical to SEQ ID NO: 87

SEQ ID NO: 188 - protein, 119 aa, synthetic construct
QVQLVQSGAEVKKPGSSVKVSCKASGTDFDKTYIHWVRQAPGQGLEWMGRISAGSGNVKYNEKFKGRVTITADTSTSTAYMELSSLRSEDTAVYYCAGSYYSYDVLDYWGQGTTVTVSS

SEQ ID NO: 189 - protein, 246 aa, synthetic construct
QVQLVQSGAEVKKPGSSVKVSCKASGTDFDKIYIHWVRQAPGQGLEWMGRISAGSGNIKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 190 - protein, 107 aa, synthetic construct; identical to SEQ ID NO: 87

SEQ ID NO: 191 - protein, 119 aa, synthetic construct
QVQLVQSGAEVKKPGSSVKVSCKASGTDFDKIYIHWVRQAPGQGLEWMGRISAGSGNIKYNEKFKGRVTITADTSTSTAYMELSSLRSEDTAVYYCAGSYYSYDVLDYWGQGTTVTVSS

SEQ ID NO: 192 - protein, 246 aa, synthetic construct
QVQLVQSGAEVKKPGSSVKVSCKASGTDFDKIYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWYQQKPGKAPKALIYSSSHRYKGVPSRFSGSGSGTEFTLTISSLQPEDFATYFCQQFKSYPLTFGQGTKLEIK

SEQ ID NO: 193 - protein, 107 aa, synthetic construct; identical to SEQ ID NO: 102

SEQ ID NO: 194 - protein, 119 aa, synthetic construct; identical to SEQ ID NO: 85

SEQ ID NO: 195 - protein, 246 aa, synthetic construct
QVQLVQSGAEVKKPGSSVKVSCKASGGTFKLTYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 196 - protein, 107 aa, synthetic construct; identical to SEQ ID NO: 87

SEQ ID NO: 197 - protein, 119 aa, synthetic construct
QVQLVQSGAEVKKPGSSVKVSCKASGGTFKLTYIHWVRQAPGQGLEWMGRVSAGSGNVKYNEKFKGRVTITADTSTSTAYMELSSLRSEDTAVYYCAGSYYSYDVLDYWGQGTTVTVSS

SEQ ID NO: 198 - protein, 246 aa, synthetic construct
QVQLVQSGAEVKKPGSSVKVSCKASGTDFKLTYIHWVRQAPGQGLEWMGRISPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNRVAWHQQKPGKAPKALIYSSSHRYKGVPSRFSGSGSGTEFTLTISSLQPEDFATYFCQQFKSYPLTFGQGTKLEIK

SEQ ID NO: 199 - protein, 107 aa, synthetic construct
DIQMTQSPSFLSASVGDRVTITCKASQNVDNRVAWHQQKPGKAPKALIYSSSHRYKGVPSRFSGSGSGTEFTLTISSLQPEDFATYFCQQFKSYPLTFGQGTKLEIK

SEQ ID NO: 200 - protein, 119 aa, synthetic construct
QVQLVQSGAEVKKPGSSVKVSCKASGTDFKLTYIHWVRQAPGQGLEWMGRISPGSGNVKYNEKFKGRVTITADTSTSTAYMELSSLRSEDTAVYYCAGSYYSYDVLDYWGQGTTVTVSS

SEQ ID NO: 201 - protein, 246 aa, synthetic construct
QVQLVQSGAEVKKPGSSVKVSCKASGHDFDKFYIHWVRQAPGQGLEWMGRVSAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNRVAWYQQKPGKAPKALIYSSSHRYKGVPSRFSGSGSGTEFTLTISSLQPEDFATYFCQQFKSYPLTFGQGTKLEIK

SEQ ID NO: 202 - protein, 107 aa, synthetic construct; identical to SEQ ID NO: 90

SEQ ID NO: 203 - protein, 119 aa, synthetic construct
QVQLVQSGAEVKKPGSSVKVSCKASGHDFDKFYIHWVRQAPGQGLEWMGRVSAGSGNTKYNEKFKGRVTITADTSTSTAYMELSSLRSEDTAVYYCAGSYYSYDVLDYWGQGTTVTVSS

SEQ ID NO: 204 - protein, 246 aa, synthetic construct
QVQLVQSGAEVKKPGSSVKVSCKASGTDFDKFYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVVWYQQKPGKAPKALIYSSSHRYKGVPSRFSGSGSGTEFTLTISSLQPEDFATYFCQQFKSYPLTFGQGTKLEIK

SEQ ID NO: 205 - protein, 119 aa, synthetic construct
QVQLVQSGAEVKKPGSSVKVSCKASGTDFDKFYIHWVRQAPGQGLEWMGRVSAGSGNVKYNEKFKGRVTITADTSTSTAYMELSSLRSEDTAVYYCAGSYYSYDVLDYWGQGTTVTVSS

SEQ ID NO: 206 - protein, 246 aa, synthetic construct
QVQLVQSGAEVKKPGSSVKVSCKASGYSFTTYYIHWVRQAPGQGLEWMGWFSAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 207 - protein, 119 aa, synthetic construct
QVQLVQSGAEVKKPGSSVKVSCKASGYSFTTYYIHWVRQAPGQGLEWMGWFSAGSGNTKYNEKFKGRVTITADTSTSTAYMELSSLRSEDTAVYYCAVSYYSYDVLDYWGQGTTVTVSS

SEQ ID NO: 208 - protein, 246 aa, synthetic construct
QVQLVQSGAEVKKPGSSVKVSCKASGYSFTTYYIHWVRQAPGQGLEWMGWFSAGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 209 - protein, 119 aa, synthetic construct
QVQLVQSGAEVKKPGSSVKVSCKASGYSFTTYYIHWVRQAPGQGLEWMGWFSAGSGNTKYNEKFKGRVTITADTSTSTAYMELSSLRSEDTAVYYCAGSYYSYDVLDYWGQGTTVTVSS

SEQ ID NO: 210 - protein, 246 aa, synthetic construct
QVQLVQSGAEVKKPGSSVKVSCKASGYSFTTYYIHWVRQAPGQGLEWMGWFFPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 211 - protein, 119 aa, synthetic construct
QVQLVQSGAEVKKPGSSVKVSCKASGYSFTTYYIHWVRQAPGQGLEWMGWFFPGSGNTKYNEKFKGRVTITADTSTSTAYMELSSLRSEDTAVYYCAGSYYSYDVLDYWGQGTTVTVSS

SEQ ID NO: 212 - protein, 246 aa, synthetic construct
QVQLVQSGAEVKKPGSSVKVSCKASGYSFTTYYIHWVRQAPGQGLEWMGWFSPGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 213 - protein, 119 aa, synthetic construct
QVQLVQSGAEVKKPGSSVKVSCKASGYSFTTYYIHWVRQAPGQGLEWMGWFSPGSGNTKYNEKFKGRVTITADTSTSTAYMELSSLRSEDTAVYYCAVSYYSYDVLDYWGQGTTVTVSS

SEQ ID NO: 214 - protein, 246 aa, synthetic construct
QVQLVQSGAEVKKPGSSVKVSCKASGYSFTTYYIHWVRQAPGQGLEWMGWFSPGSGNTKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 215 - protein, 119 aa, synthetic construct
QVQLVQSGAEVKKPGSSVKVSCKASGYSFTTYYIHWVRQAPGQGLEWMGWFSPGSGNTKYNEKFKGRVTITADTSTSTAYMELSSLRSEDTAVYYCAGSYYSYDVLDYWGQGTTVTVSS

SEQ ID NO: 216 - protein, 246 aa, synthetic construct
QVQLVQSGAEVKKPGSSVKVSCKASGYSFTTYYIHWVRQAPGQGLEWMGRIFPGSGNIKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 217 - protein, 119 aa, synthetic construct
QVQLVQSGAEVKKPGSSVKVSCKASGYSFTTYYIHWVRQAPGQGLEWMGRIFPGSGNIKYNEKFKGRVTITADTSTSTAYMELSSLRSEDTAVYYCAGSYYSYDVLDYWGQGTTVTVSS

SEQ ID NO: 218 - protein, 246 aa, synthetic construct
QVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSIYSAGVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 219 - protein, 119 aa, synthetic construct
QVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSIYSAGVLDYWGQGTTVTVSS

SEQ ID NO: 220 - protein, 246 aa, synthetic construct
QVQLVQSGAEVKKPGSSVKVSCKASGYSFTLGYIHWVRQAPGQGLEWMGWFFPGSGNIKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 221 - protein, 119 aa, synthetic construct
QVQLVQSGAEVKKPGSSVKVSCKASGYSFTLGYIHWVRQAPGQGLEWMGWFFPGSGNIKYNEKFKGRVTITADTSTSTAYMELSSLRSEDTAVYYCAGSYYSYDVLDYWGQGTTVTVSS

SEQ ID NO: 222 - protein, 246 aa, synthetic construct
QVQLVQSGAEVKKPGSSVKVSCKASGYSFRLTYIHWVRQAPGQGLEWMGWFFPGSGNIKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 223 - protein, 119 aa, synthetic construct
QVQLVQSGAEVKKPGSSVKVSCKASGYSFRLTYIHWVRQAPGQGLEWMGWFFPGSGNIKYNEKFKGRVTITADTSTSTAYMELSSLRSEDTAVYYCAGSYYSYDVLDYWGQGTTVTVSS

SEQ ID NO: 224 - protein, 246 aa, synthetic construct
QVQLVQSGAEVKKPGSSVKVSCKASGYSFHNWYIHWVRQAPGQGLEWMGWFFPGSGNIKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 225 - protein, 119 aa, synthetic construct
QVQLVQSGAEVKKPGSSVKVSCKASGYSFHNWYIHWVRQAPGQGLEWMGWFFPGSGNIKYNEKFKGRVTITADTSTSTAYMELSSLRSEDTAVYYCAGSYYSYDVLDYWGQGTTVTVSS

SEQ ID NO: 226-229 - skipped (4 entries)

SEQ ID NO: 230 - protein, 107 aa, synthetic construct
  VARIANT 30: note = G, E, A or D
  VARIANT 31: note = N or D
  VARIANT 32: note = R or K
  VARIANT 36: note = Y or H
  VARIANT 56: note = K or S
DIQMTQSPSFLSASVGDRVTITCKASQNVXXXVAWXQQKPGKAPKALIYSSSHRYXGVPSRFSGSGSGTEFTLTISSLQPEDFATYFCQQFKSYPLTFGQGTKLEIK

SEQ ID NO: 231 - protein, 119 aa, synthetic construct
  VARIANT 27: note = H, T, G or Y
  VARIANT 28: note = D, T or S
  VARIANT 30: note = H, R, D, K or T
  VARIANT 31: note = L, D, K, T or N
  VARIANT 32: note = W, F, T, I, Y or G
  VARIANT 50: note = R or W
  VARIANT 51: note = V, I or F
  VARIANT 52: note = F, S, or Y
  VARIANT 53: note = A or P
  VARIANT 57: note = N or S
  VARIANT 58: note = T, V, Y or I
  VARIANT 59: note = K or R
  VARIANT 98: note = G or V
  VARIANT 100: note = Y or I
  VARIANT 103: note = Y or A
  VARIANT 104: note = D or G
QVQLVQSGAEVKKPGSSVKVSCKASGXXFXXXYIHWVRQAPGQGLEWMGXXXXGSGXXXYNEKFKGRVTITADTSTSTAYMELSSLRSEDTAVYYCAXSXYSXXVLDYWGQGTTVTVSS

SEQ ID NO: 232 - protein, 107 aa, synthetic construct
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPRTFGGGTKVEIK

SEQ ID NO: 233 - protein, 120 aa, synthetic construct
QVQLKESGPGLVAPSQSLSITCTVSGFSLTAYGVNWVRQPPGKGLEWLGMIWGDGNTDYNSALKSRLSISKDNSKSQVFLKMNSLQTDDTARYYCARDRVTATLYAMDYWGQGTSVTVSS

SEQ ID NO: 234-1003 - skipped (770 entries)

SEQ ID NO: 1004 - protein, 721 aa, synthetic construct
METDTLLLWVLLLWVPGSTGQVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIKGGGGSQVTLRESGPALVKPTQTLTLTCTFSGFSLSTSGMGVGWIRQPPGKALEWLAHIWWDDDKRYNPALKSRLTISKDTSKNQVFLTMTNMDPVDTATYYCARMELWSYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1005 - protein, 233 aa, synthetic construct
METPAQLLFLLLLWLPDTTGENVLTQSPATLSLSPGERATLSCSASSSVSYMHWYQQKPGQAPRLLIYDTSKLASGIPARFSGSGSGTDHTLTISSLEPEDFAVYYCFQGSVYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1006 - protein, 721 aa, synthetic construct
METDTLLLWVLLLWVPGSTGQVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIKGGGGSQVQLVESGGGVVQPGRSLRLSCAASGIDFSRYWMSWVRQAPGKGLEWVGEINPDSSTINYAPSLKDRFTISRDNSKNTLYLQMSSLRAEDTAVYYCASLYYDYGDAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 1007 - protein, 234 aa, synthetic construct
METDTLLLWVLLLWVPGSTGDIQMTQSPSSLSASVGDRVTITCKASQSVDSNVAWYQQKPEKAPKALIFSASLRFSGVPSRFSGSGSGTDFTLTISSLQPEDFATYFCQQYNNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1008-1099 - skipped (92 entries)

SEQ ID NO: 1100 - protein, 119 aa, synthetic construct; identical to SEQ ID NO: 9

SEQ ID NO: 1101 - protein, 106 aa, synthetic construct
QSVLTQPPSVSEAPRQRVTISCKASQNVGINVVWHQQLPGKAPKALIYSSSHRYSGVSDRFSGSGSGTSFSLAISGLQSEDEADYFCQQFKSYPLTFGTGTKVTVL

SEQ ID NO: 1102-1140 - skipped (39 entries)

SEQ ID NO: 1141 - protein, 5 aa, synthetic construct
SYAMS

SEQ ID NO: 1142 - protein, 17 aa, synthetic construct
HISNGGDYIYYADTVKG

SEQ ID NO: 1143 - protein, 12 aa, synthetic construct
PSYYSDPWFFDV

SEQ ID NO: 1144 - protein, 7 aa, synthetic construct
GFTFRSY

SEQ ID NO: 1145 - protein, 6 aa, synthetic construct
SNGGDY

SEQ ID NO: 1146 - protein, 10 aa, synthetic construct
GFTFRSYAMS

SEQ ID NO: 1147 - protein, 10 aa, synthetic construct
SAGSSVSFMH

SEQ ID NO: 1148 - protein, 7 aa, synthetic construct
DTSKLAS

SEQ ID NO: 1149 - protein, 9 aa, synthetic construct
LQGSGFPLT

SEQ ID NO: 1150 - protein, 6 aa, synthetic construct
GSSVSF

SEQ ID NO: 1151 - protein, 106 aa, synthetic construct
ENVLTQSPAIMSASPGEKVTMTCSAGSSVSFMHWYQQKSSTSPKLWIYDTSKLASGVPGRFSGSGSGNSFSLTISSMEAEDVAIYYCLQGSGFPLTFGSGTKLEIK

SEQ ID NO: 1152 - protein, 121 aa, synthetic construct
DVKLVESGEGLVKPGGSLKLSCAASGFTFRSYAMSWVRQTPEKRLEWVAHISNGGDYIYYADTVKGRFTISRDNARNTLYLQMSSLKSEDTAMYYCTRPSYYSDPWFFDVWGTGTTVTVSS

SEQ ID NO: 1153 - protein, 121 aa, synthetic construct
EVQLVESGGGLVQPGGSLRLSCAASGFTFRSYAMSWVRQAPGKGLEWVAHISNGGDYIYYADTVKGRFTISRDNAKNSLYLQMNSLRAEDTAVYYCTRPSYYSDPWFFDVWGQGTTVTVSS

SEQ ID NO: 1154 - protein, 121 aa, synthetic construct
QVQLVESGGGVVQPGRSLRLSCAASGFTFRSYAMSWVRQAPGKGLEWVAHISNGGDYIYYADTVKGRFTISRDNSKNTLYLQMSSLRAEDTAVYYCTRPSYYSDPWFFDVWGQGTTVTVSS

SEQ ID NO: 1155 - protein, 121 aa, synthetic construct
EVQLVESGGGLVQPGGSLRLSCAASGFTFRSYAMSWVRQAPGKGLEWVAHISNGGDYIYYADTVKGRFTISRDNSKNTLYLQMNSLRAEDTAVYYCTRPSYYSDPWFFDVWGQGTTVTVSS

SEQ ID NO: 1156 - protein, 121 aa, synthetic construct
QVQLVQSGSELKKPGASVKVSCKASGFTFRSYAMSWVRQAPGQGLEWVAHISNGGDYIYYADTVKGRFVISRDNSVNTLYLQISSLKAEDTAVYYCTRPSYYSDPWFFDVWGQGTTVTVSS

SEQ ID NO: 1157 - protein, 121 aa, synthetic construct
QVQLVQSGAEVKKPGASVKVSCKASGFTFRSYAMSWVRQAPGQRLEWVAHISNGGDYIYYADTVKGRFTITRDNSANTLYMELSSLRSEDTAVYYCTRPSYYSDPWFFDVWGQGTTVTVSS

SEQ ID NO: 1158 - protein, 106 aa, synthetic construct
ENVLTQSPATLSLSPGERATLSCSAGSSVSFMHWYQQKPGQAPKLLIYDTSKLASGIPARFSGSGSGNDFTLTISSLEPEDFAVYYCLQGSGFPLTFGQGTKLEIK

SEQ ID NO: 1159 - protein, 106 aa, synthetic construct
ENVLTQSPDFQSVTPKEKVTITCSAGSSVSFMHWYQQKPDQSPKLLIYDTSKLASGVPSRFSGSGSGNDFTLTINSLEAEDAATYYCLQGSGFPLTFGQGTKLEIK

SEQ ID NO: 1160 - protein, 106 aa, synthetic construct
DNQLTQSPSSLSASVGDRVTITCSAGSSVSFMHWYQQKPGKVPKLLIYDTSKLASGVPSRFSGSGSGNDFTLTISSLQPEDVATYYCLQGSGFPLTFGQGTKLEIK

SEQ ID NO: 1161 - protein, 106 aa, synthetic construct
ANQLTQSPSSLSASVGDRVTITCSAGSSVSFMHWYQQKPGKAPKLLIYDTSKLASGVPSRFSGSGSGNDFTLTISSLQPEDFATYYCLQGSGFPLTFGQGTKLEIK

SEQ ID NO: 1162 - protein, 106 aa, synthetic construct
DNVLTQSPDSLAVSLGERATINCSAGSSVSFMHWYQQKPGQPPKLLIYDTSKLASGVPDRFSGSGSGNDFTLTISSLQAEDVAVYYCLQGSGFPLTFGQGTKLEIK

SEQ ID NO: 1163-1184 - skipped (22 entries)

SEQ ID NO: 1185 - protein, 5 aa, synthetic construct
GSWMN

SEQ ID NO: 1186 - protein, 17 aa, synthetic construct
RIYPGDGDTDYSGKFKG

SEQ ID NO: 1187 - protein, 12 aa, synthetic construct
SGYFNYVPVFDY

SEQ ID NO: 1188 - protein, 7 aa, synthetic construct
GYTFSGS

SEQ ID NO: 1189 - protein, 6 aa, synthetic construct
YPGDGD

SEQ ID NO: 1190 - protein, 10 aa, synthetic construct
GYTFSGSWMN

SEQ ID NO: 1191 - protein, 10 aa, synthetic construct
SANSTVGYIH

SEQ ID NO: 1192 - protein, 7 aa, synthetic construct
TTSNLAS

SEQ ID NO: 1193 - protein, 8 aa, synthetic construct
HQWSFYPT

SEQ ID NO: 1194 - protein, 6 aa, synthetic construct
NSTVGY

SEQ ID NO: 1195 - protein, 105 aa, synthetic construct
QIVLTQSPAIMSASLGEEIALTCSANSTVGYIHWYQQKSGTSPKLLIYTTSNLASGVPSRFSGSGSGTFYSLTISSVEAEDAADYFCHQWSFYPTFGGGTKLEIK

SEQ ID NO: 1196 - protein, 121 aa, synthetic construct
QIQLQQSGPEVVKPGASVQISCKASGYTFSGSWMNWVKQRPGKGLEWIGRIYPGDGDTDYSGKFKGRATLTADKSSSTAYMRLSSLTSEDSAVYFCARSGYFNYVPVFDYWGQGTTLSVSS

SEQ ID NO: 1197 - protein, 121 aa, synthetic construct
QIQLVQSGAEVKKPGASVKVSCKASGYTFSGSWMNWVRQAPGQGLEWIGRIYPGDGDTDYSGKFKGRATLTADKSTSTAYMELSSLRSEDTAVYYCARSGYFNYVPVFDYWGQGTTVTVSS

SEQ ID NO: 1198 - protein, 121 aa, synthetic construct
QIQLVQSGAEVKKPGSSVKVSCKASGYTFSGSWMNWVRQAPGQGLEWIGRIYPGDGDTDYSGKFKGRATLTADKSTSTAYMELSSLRSEDTAVYYCARSGYFNYVPVFDYWGQGTTVTVSS

SEQ ID NO: 1199 - protein, 121 aa, synthetic construct
EIQLVQSGAEVKKPGESLKISCKASGYTFSGSWMNWVRQMPGKGLEWIGRIYPGDGDTDYSGKFKGQATLSADKSISTAYLQWSSLKASDTAMYYCARSGYFNYVPVFDYWGQGTTVTVSS

SEQ ID NO: 1200 - protein, 121 aa, synthetic construct
QIQLVQSGSELKKPGASVKVSCKASGYTFSGSWMNWVRQAPGQGLEWIGRIYPGDGDTDYSGKFKGRAVLSADKSVSTAYLQISSLKAEDTAVYYCARSGYFNYVPVFDYWGQGTTVTVSS

SEQ ID NO: 1201 - protein, 121 aa, synthetic construct
QIQLVQSGSELKKPGASVKVSCKASGYTFSGSWMNWVRQAPGQGLEWIGRIYPGDGDTDYSGKFKGRAVLSADKSVSMAYLQISSLKAEDTAVYYCARSGYFNYVPVFDYWGQGTTVTVSS

SEQ ID NO: 1202 - protein, 121 aa, synthetic construct
EIQLVESGGGLVQPGRSLRLSCTASGYTFSGSWMNWVRQAPGKGLEWIGRIYPGDGDTDYSGKFKGRATLSADKSKSIAYLQMNSLKTEDTAVYYCARSGYFNYVPVFDYWGQGTTVTVSS

SEQ ID NO: 1203 - protein, 105 aa, synthetic construct
EIVLTQSPATLSLSPGERATLSCSANSTVGYIHWYQQKPGQAPKLLIYTTSNLASGIPARFSGSGSGTDYTLTISSLEPEDFAVYFCHQWSFYPTFGQGTKLEIK

SEQ ID NO: 1204 - protein, 105 aa, synthetic construct
DIQLTQSPSFLSASVGDRVTITCSANSTVGYIHWYQQKPGKAPKLLIYTTSNLASGVPSRFSGSGSGTEYTLTISSLQPEDFATYFCHQWSFYPTFGQGTKLEIK

SEQ ID NO: 1205 - protein, 105 aa, synthetic construct
EIVLTQSPATLSLSPGERATLSCSANSTVGYIHWYQQKPGQAPKLLIYTTSNLASGIPARFSGSGPGTDYTLTISSLEPEDFAVYFCHQWSFYPTFGQGTKLEIK

SEQ ID NO: 1206 - protein, 105 aa, synthetic construct
DIVLTQSPDSLAVSLGERATINCSANSTVGYIHWYQQKPGQPPKLLIYTTSNLASGVPDRFSGSGSGTDYTLTISSLQAEDVAVYFCHQWSFYPTFGQGTKLEIK

SEQ ID NO: 1207 - protein, 105 aa, synthetic construct
EIVLTQSPDFQSVTPKEKVTITCSANSTVGYIHWYQQKPDQSPKLLIYTTSNLASGVPSRFSGSGSGTDYTLTINSLEAEDAATYFCHQWSFYPTFGQGTKLEIK

SEQ ID NO: 1208-1287 - skipped (80 entries)

SEQ ID NO: 1288 - protein, 5 aa, synthetic construct
SYGMS

SEQ ID NO: 1289 - protein, 17 aa, synthetic construct
LISSGGSYTYYTDSVKG

SEQ ID NO: 1290 - protein, 8 aa, synthetic construct
HGGNFFDY

SEQ ID NO: 1291 - protein, 7 aa, synthetic construct
GFTFRSY

SEQ ID NO: 1292 - protein, 6 aa, synthetic construct
SSGGSY

SEQ ID NO: 1293 - protein, 10 aa, synthetic construct
GFTFRSYGMS

SEQ ID NO: 1294 - protein, 10 aa, synthetic construct
SVSSSVSYMH

SEQ ID NO: 1295 - protein, 7 aa, synthetic construct
DTSKLAS

SEQ ID NO: 1296 - protein, 10 aa, synthetic construct
QQWSSNPQYT

SEQ ID NO: 1297 - protein, 6 aa, synthetic construct
SSSVSY

SEQ ID NO: 1298 - protein, 5 aa, synthetic construct
SSWMN

SEQ ID NO: 1299 - protein, 17 aa, synthetic construct
RIYPGDGDTKYNGKFKG

SEQ ID NO: 1300 - protein, 10 aa, synthetic construct
RGTGGWYFDV

SEQ ID NO: 1301 - protein, 10 aa, synthetic construct
RGTGGWYFDV

SEQ ID NO: 1302 - protein, 7 aa, synthetic construct
GYAFSSS

SEQ ID NO: 1303 - protein, 6 aa, synthetic construct
YPGDGD

SEQ ID NO: 1304 - protein, 10 aa, synthetic construct
GYAFSSSWMN

SEQ ID NO: 1305 - protein, 15 aa, synthetic construct
RASESVDSSGNSFMH

SEQ ID NO: 1306 - protein, 7 aa, synthetic construct
RASNLES

SEQ ID NO: 1307 - protein, 9 aa, synthetic construct
QQSFDDPFT

SEQ ID NO: 1308 - protein, 11 aa, synthetic construct
SESVDSSGNSF

SEQ ID NO: 1309 - protein, 246 aa, synthetic construct
QVQLVQSGAEVKKPGSSVKVSCKASGHDFDKFYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGNRVAWYQQKPGKAPKALIYSSSHRYKGVPSRFSGSGSGTEFTLTISSLQPEDFATYFCQQFKSYPLTFGQGTKLEIK

SEQ ID NO: 1310 - protein, 119 aa, synthetic construct; identical to SEQ ID NO: 94

SEQ ID NO: 1311 - protein, 119 aa, synthetic construct
QVQLVQSGAEVKKPGSSVKVSCKASGTDFKLTYIHWVRQAPGQGLEWMGRIFPGSGNVKYNEKFKGRVTITADTSTSTAYMELSSLRSEDTAVYYCAVSYYSYDVLDYWGQGTTVTVSS

SEQ ID NO: 1312 - protein, 119 aa, synthetic construct
QVQLVQSGAEVKKPGSSVKVSCKASGHDFRLTYIHWVRQAPGQGLEWMGRISAGSGNVKYNEKFKGRVTITADTSTSTAYMELSSLRSEDTAVYYCAVSYYSYDVLDYWGQGTTVTVSS

SEQ ID NO: 1313 - protein, 107 aa, synthetic construct; identical to SEQ ID NO: 90

SEQ ID NO: 1314 - protein, 107 aa, synthetic construct; identical to SEQ ID NO: 141

SEQ ID NO: 1315 - protein, 5 aa, synthetic construct
AYGVN

SEQ ID NO: 1316 - protein, 16 aa, synthetic construct
MIWGDGNTDYNSALKS

SEQ ID NO: 1317 - protein, 12 aa, synthetic construct
DRVTATLYAMDY

SEQ ID NO: 1318 - protein, 7 aa, synthetic construct
GFSLTAY

SEQ ID NO: 1319 - protein, 5 aa, synthetic construct
WGDGN

SEQ ID NO: 1320 - protein, 10 aa, synthetic construct
GFSLTAYGVN

SEQ ID NO: 1321 - protein, 11 aa, synthetic construct
SASQGISNYLN

SEQ ID NO: 1322 - protein, 7 aa, synthetic construct
YTSSLHS

SEQ ID NO: 1323 - protein, 9 aa, synthetic construct
QQYSKLPRT

SEQ ID NO: 1324 - protein, 107 aa, synthetic construct
DIQMTQSPSSLSASVGDRVTITCSASQGISNYLNWYQQTPGKAPKLLIYYTSSLHSGVPSRFSGSGSGTDYTFTISSLQPEDIATYYCQQYSKLPRTFGQGTKLQIT

SEQ ID NO: 1325 - protein, 120 aa, synthetic construct
QVQLQESGPGLVRPSQTLSLTCTVSGFSLTAYGVNWVRQPPGRGLEWLGMIWGDGNTDYNSALKSRVTMLKDTSKNQFSLRLSSVTAADTAVYYCARDRVTATLYAMDYWGQGSLVTVSS

SEQ ID NO: 1326 - protein, 246 aa, synthetic construct
QVQLVQSGAEVKKPGSSVKVSCKASGHDFRLTYIHWVRQAPGQGLEWMGRVSAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVGDRVVWHQQKPGKAPKALIYSSSHRYKGVPSRFSGSGSGTEFTLTISSLQPEDFATYFCQQFKSYPLTFGQGTKLEIK

SEQ ID NO: 1327 - protein, 246 aa, synthetic construct
QVQLVQSGAEVKKPGSSVKVSCKASGHDFRLTYIHWVRQAPGQGLEWMGRVSAGSGNVKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVGNRVVWHQQKPGKAPKALIYSSSHRYKGVPSRFSGSGSGTEFTLTISSLQPEDFATYFCQQFKSYPLTFGQGTKLEIK

SEQ ID NO: 1328 - protein, 246 aa, synthetic construct
QVQLVQSGAEVKKPGSSVKVSCKASGHDFRLTYIHWVRQAPGQGLEWMGRIYAGSGNVKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVADRVVWHQQKPGKAPKALIYSSSHRYKGVPSRFSGSGSGTEFTLTISSLQPEDFATYFCQQFKSYPLTFGQGTKLEIK

SEQ ID NO: 1329 - protein, 246 aa, synthetic construct
QVQLVQSGAEVKKPGSSVKVSCKASGHDFKLTYIHWVRQAPGQGLEWMGRVSAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVGDRVAWHQQKPGKAPKALIYSSSHRYKGVPSRFSGSGSGTEFTLTISSLQPEDFATYFCQQFKSYPLTFGQGTKLEIK

SEQ ID NO: 1330 - protein, 246 aa, synthetic construct
QVQLVQSGAEVKKPGSSVKVSCKASGHDFKLTYIHWVRQAPGQGLEWMGRVSAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVDNRVAWHQQKPGKAPKALIYSSSHRYKGVPSRFSGSGSGTEFTLTISSLQPEDFATYFCQQFKSYPLTFGQGTKLEIK

SEQ ID NO: 1331 - protein, 246 aa, synthetic construct
QVQLVQSGAEVKKPGSSVKVSCKASGHDFRLTYIHWVRQAPGQGLEWMGRVSAGSGNVKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVADRVVWHQQKPGKAPKALIYSSSHRYKGVPSRFSGSGSGTEFTLTISSLQPEDFATYFCQQFKSYPLTFGQGTKLEIK

SEQ ID NO: 1332 - protein, 246 aa, synthetic construct
QVQLVQSGAEVKKPGSSVKVSCKASGHDFRLTYIHWVRQAPGQGLEWMGRVYAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVEDRVVWHQQKPGKAPKALIYSSSHRYKGVPSRFSGSGSGTEFTLTISSLQPEDFATYFCQQFKSYPLTFGQGTKLEIK

SEQ ID NO: 1333 - protein, 246 aa, synthetic construct
QVQLVQSGAEVKKPGSSVKVSCKASGHDFKLTYIHWVRQAPGQGLEWMGRVSAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVADRVVWHQQKPGKAPKALIYSSSHRYKGVPSRFSGSGSGTEFTLTISSLQPEDFATYFCQQFKSYPLTFGQGTKLEIK

SEQ ID NO: 1334 - protein, 246 aa, synthetic construct
QVQLVQSGAEVKKPGSSVKVSCKASGHDFKLTYIHWVRQAPGQGLEWMGRISAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVGDRVVWHQQKPGKAPKALIYSSSHRYKGVPSRFSGSGSGTEFTLTISSLQPEDFATYFCQQFKSYPLTFGQGTKLEIK

SEQ ID NO: 1335 - protein, 246 aa, synthetic construct
QVQLVQSGAEVKKPGSSVKVSCKASGHDFKLTYIHWVRQAPGQGLEWMGRIYAGSGNTKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVGDRVVWHQQKPGKAPKALIYSSSHRYKGVPSRFSGSGSGTEFTLTISSLQPEDFATYFCQQFKSYPLTFGQGTKLEIK

SEQ ID NO: 1336 - protein, 246 aa, synthetic construct
QVQLVQSGAEVKKPGSSVKVSCKASGTDFKLTYIHWVRQAPGQGLEWMGRIFPGSGNVKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVDNRVAWYQQKPGKAPKALIYSSSHRYKGVPSRFSGSGSGTEFTLTISSLQPEDFATYFCQQFKSYPLTFGQGTKLEIK

SEQ ID NO: 1337 - protein, 246 aa, synthetic construct
QVQLVQSGAEVKKPGSSVKVSCKASGHDFRLTYIHWVRQAPGQGLEWMGRISAGSGNVKYNEKFKGRVTITADTSTSTAYMELSSLRSEDTAVYYCAVSYYSYDVLDYWGQGTTVTVSSGGGGSGGGGSGGGGSGGGGSDIQMTQSPSFLSASVGDRVTITCKASQNVADRVAWYQQKPGKAPKALIYSSSHRYKGVPSRFSGSGSGTEFTLTISSLQPEDFATYFCQQFKSYPLTFGQGTKLEIK

SEQ ID NO: 1338 - protein, 243 aa, synthetic construct
EVQLVESGGGLVQPGGSLRLSCAASGFTFSNFGMHWVRQAPGKGLEWVSYISSGSSTIYYADTLKGRFTISRDNSKNTLYLQMNSLRAEDTAVYYCARRGEGAMDYWGQGTTVTVSSGGGGSGGGGSGGGGSGGGGSDNQLTQSPSSLSASVGDRVTITCRASSSVNYIYWYQQKPGKAPKLLIYYTSNLAPGVPSRFSGSGSGNDYTLTISSLQPEDFATYYCQQFTSSPFTFGQGTKLEIK

SEQ ID NO: 1339 - protein, 242 aa, synthetic construct
EVQLVESGGGLVQPGGSLRLSCAASGFTFSNFGMHWVRQAPGKGLEWVSYISSGSSTIYYADTLKGRFTISRDNSKNTLYLQMNSLRAEDTAVYYCARRGEGAMDYWGQGTTVTVSSGGGGSGGGGSGGGGSGGGGSSNELTQPPSVSVSPGQTARITCRASSSVNYIYWYQQKSGQAPVLVIYYTSNLAPGIPERFSGSGSGNMYTLTISGAQVEDEADYYCQQFTSSPFTFGTGTKVTVL

SEQ ID NO: 1340 - protein, 243 aa, synthetic construct
QVQLVESGGGVVQPGRSLRLSCAASGFTFSNFGMHWVRQAPGKGLEWVAYISSGSSTIYYADTLKGRFTISRDNSKNTLYLQMNSLRAEDTAVYYCARRGEGAMDYWGQGTTVTVSSGGGGSGGGGSGGGGSGGGGSDNQLTQSPSFLSASVGDRVTITCRASSSVNYIYWYQQKPGKAPKLLIYYTSNLAPGVPSRFSGSGSGNEYTLTISSLQPEDFATYYCQQFTSSPFTFGQGTKLEIK

SEQ ID NO: 1341 - protein, 243 aa, synthetic construct
QVQLVESGGGVVQPGRSLRLSCAASGFTFSNFGMHWVRQAPGKGLEWVAYISSGSSTIYYADTLKGRFTISRDNSKNTLYLQMNSLRAEDTAVYYCARRGEGAMDYWGQGTTVTVSSGGGGSGGGGSGGGGSGGGGSDNQLTQSPSSLSASVGDRVTITCRASSSVNYIYWYQQKPGKAPKLLIYYTSNLAPGVPSRFSGSGSGNDYTLTISSLQPEDFATYYCQQFTSSPFTFGQGTKLEIK

SEQ ID NO: 1342 - protein, 242 aa, synthetic construct
QVQLVESGGGVVQPGRSLRLSCAASGFTFSNFGMHWVRQAPGKGLEWVAYISSGSSTIYYADTLKGRFTISRDNSKNTLYLQMNSLRAEDTAVYYCARRGEGAMDYWGQGTTVTVSSGGGGSGGGGSGGGGSGGGGSSNELTQPPSVSVSPGQTARITCRASSSVNYIYWYQQKSGQAPVLVIYYTSNLAPGIPERFSGSGSGNMYTLTISGAQVEDEADYYCQQFTSSPFTFGTGTKVTVL

SEQ ID NO: 1343 - skipped

SEQ ID NO: 1344 - protein, 119 aa, synthetic construct
QVQLVQSGAEVKKPGSSVKVSCKASGHDFRLTYIHWVRQAPGQGLEWMGRVSAGSGNTKYNEKFKGRVTITADTSTSTAYMELSSLRSEDTAVYYCAVSYYSYDVLDYWGQGTTVTVSS

SEQ ID NO: 1345 - skipped

SEQ ID NO: 1346 - protein, 119 aa, synthetic construct
QVQLVQSGAEVKKPGSSVKVSCKASGHDFRLTYIHWVRQAPGQGLEWMGRVSAGSGNVKYNEKFKGRVTITADTSTSTAYMELSSLRSEDTAVYYCAVSYYSYDVLDYWGQGTTVTVSS

SEQ ID NO: 1347 - protein, 107 aa, synthetic construct
DIQMTQSPSFLSASVGDRVTITCKASQNVGNRVVWHQQKPGKAPKALIYSSSHRYKGVPSRFSGSGSGTEFTLTISSLQPEDFATYFCQQFKSYPLTFGQGTKLEIK

SEQ ID NO: 1348 - protein, 119 aa, synthetic construct
QVQLVQSGAEVKKPGSSVKVSCKASGHDFRLTYIHWVRQAPGQGLEWMGRIYAGSGNVKYNEKFKGRVTITADTSTSTAYMELSSLRSEDTAVYYCAVSYYSYDVLDYWGQGTTVTVSS

SEQ ID NO: 1349 - protein, 107 aa, synthetic construct
DIQMTQSPSFLSASVGDRVTITCKASQNVADRVVWHQQKPGKAPKALIYSSSHRYKGVPSRFSGSGSGTEFTLTISSLQPEDFATYFCQQFKSYPLTFGQGTKLEIK

SEQ ID NO: 1350 - protein, 119 aa, synthetic construct
QVQLVQSGAEVKKPGSSVKVSCKASGHDFKLTYIHWVRQAPGQGLEWMGRVSAGSGNTKYNEKFKGRVTITADTSTSTAYMELSSLRSEDTAVYYCAVSYYSYDVLDYWGQGTTVTVSS

SEQ ID NO: 1351 - protein, 107 aa, synthetic construct; identical to SEQ ID NO: 160

SEQ ID NO: 1352 - skipped

SEQ ID NO: 1353 - protein, 107 aa, synthetic construct; identical to SEQ ID NO: 199

SEQ ID NO: 1354-1355 - skipped (2 entries)

SEQ ID NO: 1356 - protein, 119 aa, synthetic construct
QVQLVQSGAEVKKPGSSVKVSCKASGHDFRLTYIHWVRQAPGQGLEWMGRVYAGSGNTKYNEKFKGRVTITADTSTSTAYMELSSLRSEDTAVYYCAVSYYSYDVLDYWGQGTTVTVSS

SEQ ID NO: 1357 - protein, 107 aa, synthetic construct
DIQMTQSPSFLSASVGDRVTITCKASQNVEDRVVWHQQKPGKAPKALIYSSSHRYKGVPSRFSGSGSGTEFTLTISSLQPEDFATYFCQQFKSYPLTFGQGTKLEIK

SEQ ID NO: 1358-1359 - skipped (2 entries)

SEQ ID NO: 1360 - protein, 119 aa, synthetic construct
QVQLVQSGAEVKKPGSSVKVSCKASGHDFKLTYIHWVRQAPGQGLEWMGRISAGSGNTKYNEKFKGRVTITADTSTSTAYMELSSLRSEDTAVYYCAVSYYSYDVLDYWGQGTTVTVSS

SEQ ID NO: 1361 - protein, 107 aa, synthetic construct
DIQMTQSPSFLSASVGDRVTITCKASQNVGDRVVWHQQKPGKAPKALIYSSSHRYKGVPSRFSGSGSGTEFTLTISSLQPEDFATYFCQQFKSYPLTFGQGTKLEIK

SEQ ID NO: 1362 - protein, 119 aa, synthetic construct
QVQLVQSGAEVKKPGSSVKVSCKASGHDFKLTYIHWVRQAPGQGLEWMGRIYAGSGNTKYNEKFKGRVTITADTSTSTAYMELSSLRSEDTAVYYCAVSYYSYDVLDYWGQGTTVTVSS

SEQ ID NO: 1363-1364 - skipped (2 entries)

SEQ ID NO: 1365 - protein, 107 aa, synthetic construct
DIQMTQSPSFLSASVGDRVTITCRASQNVDNRLGWHQQKPGKAPKALIYSSSHRYKGVPSRFSGSGSGTEFTLTISSLQPEDFATYFCQQFKSYPLTFGQGTKLEIK

SEQ ID NO: 1366 - skipped

SEQ ID NO: 1367 - protein, 107 aa, synthetic construct
DIQMTQSPSFLSASVGDRVTITCKASQNVDNRVAWHQQKPGKAPKALIYAASSLQKGVPSRFSGSGSGTEFTLTISSLQPEDFATYFCQQFKSYPLTFGQGTKLEIK

SEQ ID NO: 1368 - skipped

SEQ ID NO: 1369 - protein, 107 aa, synthetic construct
DIQMTQSPSFLSASVGDRVTITCKASQNVDNRVAWHQQKPGKAPKALIYSSSHRYKGVPSRFSGSGSGTEFTLTISSLQPEDFATYFCLQHNSYPLTFGQGTKLEIK

SEQ ID NO: 1370 - protein, 119 aa, synthetic construct
QVQLVQSGAEVKKPGSSVKVSCKASGHDFKLTYIHWVRQAPGQGLEWMGRVSAGSGNVNYAQKFQGRVTITADTSTSTAYMELSSLRSEDTAVYYCAVSYYSYDVLDYWGQGTTVTVSS

SEQ ID NO: 1371-2169 - skipped (799 entries)

SEQ ID NO: 2170 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2171-2179 - skipped (9 entries)

SEQ ID NO: 2180 - protein, 77 aa, synthetic construct
MAPRRARGCRTLGLPALLLLLLLRPPATRGITCPPPMSVEHADIWVKSYSLYSRERYICNSGFKRKAGTSSLTECVL

SEQ ID NO: 2181-2189 - skipped (9 entries)

SEQ ID NO: 2190 - protein, 20 aa, synthetic construct
SGGSGGGGSGGGSGGGGSLQ

SEQ ID NO: 2191 - protein, 133 aa, Homo sapiens [sequence omitted]

SEQ ID NO: 2192 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 2193 - protein, 133 aa, Homo sapiens [sequence omitted]

SEQ ID NO: 2194 - protein, 138 aa, Homo sapiens
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRG

SEQ ID NO: 2195-2269 - skipped (75 entries)

SEQ ID NO: 2270 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 2271-2279 - skipped (9 entries)

SEQ ID NO: 2280 - protein, 133 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 2281-2289 - skipped (9 entries)

SEQ ID NO: 2290 - protein, 518 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 2291-2299 - skipped (9 entries)

SEQ ID NO: 2300 - protein, 340 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGSGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2301-2309 - skipped (9 entries)

SEQ ID NO: 2310 - protein, 166 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 2311-2319 - skipped (9 entries)

SEQ ID NO: 2320 - protein, 114 aa, synthetic construct
NWVNVISDLKKIEDLIQSMHIDATLYTESDVHPSCKVTAMKCFLLELQVISLASGDASIHDTVENLIILANNSLSSNGAVTESGCKECEELEEKNIKEFLQSFVHIVQMFINTS

SEQ ID NO: 2321-2999 - skipped (679 entries)

SEQ ID NO: 3000 - protein, 107 aa, synthetic construct
DIQMTQSPSFLSASVGDRVTITCSASQGISNYLNWYQQKPGKAVKLLIYYTSSLHSGVPSRFSGSGSGTEYTLTISSLQPEDFATYYCQQYSKLPRTFGGGTKVEIK

SEQ ID NO: 3001 - protein, 107 aa, synthetic construct
DIQMTQSPSSLSASVGDRVTITCSASQGISNYLNWYQQKPGKAVKLLIYYTSSLHSGVPSRFSGSGSGTDYTLTISSLQPEDFATYYCQQYSKLPRTFGGGTKVEIK

SEQ ID NO: 3002 - protein, 107 aa, synthetic construct
DIQMTQSPSSLSASVGDRVTITCSASQGISNYLNWYQQKPGKVVKLLIYYTSSLHSGVPSRFSGSGSGTDYTLTISSLQPEDVATYYCQQYSKLPRTFGGGTKVEIK

SEQ ID NO: 3003 - protein, 107 aa, synthetic construct
DIQMTQSPSSLSASVGDRVTITCSASQGISNYLNWYQQKPGQAVKLLIYYTSSLHSGVPSRFSGSGSGTDYTLTISSLQPEDVATYYCQQYSKLPRTFGGGTKVEIK

SEQ ID NO: 3004 - protein, 107 aa, synthetic construct
DIQMTQSPSSLSASVGDRVTITCSASQGISNYLNWYQQKPGKAVKLLIYYTSSLHSGVPSRFSGSGSGTDYTFTISSLQPEDIATYYCQQYSKLPRTFGGGTKVEIK

SEQ ID NO: 3005 - protein, 107 aa, synthetic construct
DIQMTQSPSSLSASVGDRVTITCSASQGISNYLNWYQQKPGKTVKLLIYYTSSLHSGIPSRFSGSGSGTDYTLTIRSLQPEDFATYYCQQYSKLPRTFGGGTKVEIK

SEQ ID NO: 3006 - protein, 107 aa, synthetic construct
AIQMTQSPSSLSASVGDRVTITCSASQGISNYLNWYQQKPGKAVKLLIYYTSSLHSGVPSRFSGSGSGTDYTLTISSLQPEDFATYYCQQYSKLPRTFGGGTKVEIK

SEQ ID NO: 3007 - protein, 107 aa, synthetic construct
DIQMTQSPSSVSASVGDRVTITCSASQGISNYLNWYQQKPGKAVKLLIYYTSSLHSGVPSRFSGSGSGTDYTLTISSLQPEDFATYYCQQYSKLPRTFGGGTKVEIK

SEQ ID NO: 3008 - protein, 107 aa, synthetic construct
DIQMTQSPSSLSASVGDRVTITCSASQGISNYLNWYQQKPGKAVKRLIYYTSSLHSGVPSRFSGSGSGTEYTLTISNLQPEDFATYYCQQYSKLPRTFGGGTKVEIK

SEQ ID NO: 3009 - protein, 107 aa, synthetic construct
AIRMTQSPFSLSASVGDRVTITCSASQGISNYLNWYQQKPAKAVKLFIYYTSSLHSGVPSRFSGSGSGTDYTLTISSLQPEDFATYYCQQYSKLPRTFGGGTKVEIK

SEQ ID NO: 3010 - protein, 107 aa, synthetic construct
DIQMTQSPSSLSASVGDRVTITCSASQGISNYLNWYQQKPGKAVKRLIYYTSSLHSGVPSRFSGSGSGTEYTLTISSLQPEDFATYYCQQYSKLPRTFGGGTKVEIK

SEQ ID NO: 3011 - protein, 107 aa, synthetic construct
DIQMTQSPSTLSASVGDRVTITCSASQGISNYLNWYQQKPGKAVKLLIYYTSSLHSGVPSRFSGSGSGTEYTLTISSLQPDDFATYYCQQYSKLPRTFGGGTKVEIK

SEQ ID NO: 3012 - protein, 107 aa, synthetic construct
DIQMTQSPSSLSASVGDRVTITCSASQGISNYLNWYQQKPGKAVKSLIYYTSSLHSGVPSRFSGSGSGTDYTLTISSLQPEDFATYYCQQYSKLPRTFGGGTKVEIK

SEQ ID NO: 3013 - protein, 107 aa, synthetic construct
DIQMTQSPSSLSASVGDRVTITCSASQGISNYLNWYQQKPGKAVKSLIYYTSSLHSGVPSKFSGSGSGTDYTLTISSLQPEDFATYYCQQYSKLPRTFGGGTKVEIK

SEQ ID NO: 3014 - protein, 107 aa, synthetic construct
DIQMTQSPSSLSASVGDRVTITCSASQGISNYLNWYQQKPEKAVKSLIYYTSSLHSGVPSRFSGSGSGTDYTLTISSLQPEDFATYYCQQYSKLPRTFGGGTKVEIK

SEQ ID NO: 3015 - protein, 107 aa, synthetic construct
DIQMTQSPSAMSASVGDRVTITCSASQGISNYLNWYQQKPGKVVKRLIYYTSSLHSGVPSRFSGSGSGTEYTLTISSLQPEDFATYYCQQYSKLPRTFGGGTKVEIK

SEQ ID NO: 3016 - protein, 107 aa, synthetic construct
DIVMTQSPDSLAVSLGERATINCSASQGISNYLNWYQQKPGQPVKLLIYYTSSLHSGVPDRFSGSGSGTDYTLTISSLQAEDVAVYYCQQYSKLPRTFGGGTKVEIK

SEQ ID NO: 3017 - protein, 107 aa, synthetic construct
EIVMTQSPGTLSLSPGERATLSCSASQGISNYLNWYQQKPGQAVKLLIYYTSSLHSGIPDRFSGSGSGTDYTLTISRLEPEDFAVYYCQQYSKLPRTFGGGTKVEIK

SEQ ID NO: 3018 - protein, 107 aa, synthetic construct
EIVMTQSPPTLSLSPGERVTLSCSASQGISNYLNWYQQKPGQAVKLLIYYTSSLHSGIPARFSGSGSGTDYTLTISSLQPEDFAVYYCQQYSKLPRTFGGGTKVEIK

SEQ ID NO: 3019 - protein, 107 aa, synthetic construct
EIVMTQSPPTLSLSPGERVTLSCSASQGISNYLNWYQQKPGQAVKLLIYYTSSLHSSIPARFSGSGSGTDYTLTISSLQPEDFAVYYCQQYSKLPRTFGGGTKVEIK

SEQ ID NO: 3020 - protein, 107 aa, synthetic construct
EIVMTQSPATLSLSPGERATLSCSASQGISNYLNWYQQKPGQAVKLLIYYTSSLHSGIPARFSGSGSGTDYTLTISSLEPEDFAVYYCQQYSKLPRTFGGGTKVEIK

SEQ ID NO: 3021 - protein, 107 aa, synthetic construct
EIVMTQSPATLSLSPGERATLSCSASQGISNYLNWYQQKPGQAVKLLIYYTSSLHSGIPARFSGSGSGTDYTLTISRLEPEDFAVYYCQQYSKLPRTFGGGTKVEIK

SEQ ID NO: 3022 - protein, 107 aa, synthetic construct
EIVMTQSPATLSLSPGERATLSCSASQGISNYLNWYQQKPGQAVKLLIYYTSSLHSGIPDRFSGSGSGTDYTLTISRLEPEDFAVYYCQQYSKLPRTFGGGTKVEIK

SEQ ID NO: 3023 - protein, 107 aa, synthetic construct
EIVMTQSPATLSLSPGERATLSCSASQGISNYLNWYQQKPGLAVKLLIYYTSSLHSGIPDRFSGSGSGTDYTLTISRLEPEDFAVYYCQQYSKLPRTFGGGTKVEIK

SEQ ID NO: 3024 - protein, 107 aa, synthetic construct
DIQMIQSPSFLSASVGDRVSIICSASQGISNYLNWYLQKPGKSVKLFIYYTSSLHSGVSSRFSGRGSGTDYTLTIISLKPEDFAAYYCQQYSKLPRTFGGGTKVEIK

SEQ ID NO: 3025 - protein, 107 aa, synthetic construct
EIVMTQSPATLSLSPGERATLSCSASQGISNYLNWYQQKPGQAVKLLIYYTSSLHSGIPARFSGSGSGTDYTLTISSLQPEDFAVYYCQQYSKLPRTFGGGTKVEIK

SEQ ID NO: 3026 - protein, 107 aa, synthetic construct
EIVMTQSPATLSLSPGERATLSCSASQGISNYLNWYQQKPGQAVKLLIYYTSSLHSGIPARFSGSGPGTDYTLTISSLEPEDFAVYYCQQYSKLPRTFGGGTKVEIK

SEQ ID NO: 3027 - protein, 107 aa, synthetic construct
DIVMTQTPLSLSVTPGQPASISCSASQGISNYLNWYLQKPGQSVKLLIYYTSSLHSGVPDRFSGSGSGTDYTLKISRVEAEDVGVYYCQQYSKLPRTFGGGTKVEIK

SEQ ID NO: 3028 - protein, 107 aa, synthetic construct
DIVMTQTPLSLSVTPGQPASISCSASQGISNYLNWYLQKPGQPVKLLIYYTSSLHSGVPDRFSGSGSGTDYTLKISRVEAEDVGVYYCQQYSKLPRTFGGGTKVEIK

SEQ ID NO: 3029 - protein, 107 aa, synthetic construct
DIVMTQSPAFLSVTPGEKVTITCSASQGISNYLNWYQQKPDQAVKLLIYYTSSLHSGVPSRFSGSGSGTDYTFTISSLEAEDAATYYCQQYSKLPRTFGGGTKVEIK

SEQ ID NO: 3030 - protein, 107 aa, synthetic construct
DIVMTQSPLSLPVTPGEPASISCSASQGISNYLNWYLQKPGQSVKLLIYYTSSLHSGVPDRFSGSGSGTDYTLKISRVEAEDVGVYYCQQYSKLPRTFGGGTKVEIK

SEQ ID NO: 3031 - protein, 107 aa, synthetic construct
DIVMTQTPLSLPVTPGEPASISCSASQGISNYLNWYLQKPGQSVKLLIYYTSSLHSGVPDRFSGSGSGTDYTLKISRVEAEDVGVYYCQQYSKLPRTFGGGTKVEIK

SEQ ID NO: 3032 - protein, 107 aa, synthetic construct
EIVMTQSPATLSVSPGERATLSCSASQGISNYLNWYQQKPGQAVKLLIYYTSSLHSGIPARFSGSGSGTEYTLTISILQSEDFAVYYCQQYSKLPRTFGGGTKVEIK

SEQ ID NO: 3033 - protein, 107 aa, synthetic construct
EIVMTQSPATLSVSPGERATLSCSASQGISNYLNWYQQKPGQAVKLLIYYTSSLHSGIPARFSGSGSGTEYTLTISSLQSEDFAVYYCQQYSKLPRTFGGGTKVEIK

SEQ ID NO: 3034 - protein, 107 aa, synthetic construct
DIVMTQSPLSLPVTLGQPASISCSASQGISNYLNWYQQRPGQSVKRLIYYTSSLHSGVPDRFSGSGSGTDYTLKISRVEAEDVGVYYCQQYSKLPRTFGGGTKVEIK

SEQ ID NO: 3035 - protein, 107 aa, synthetic construct
EITMTQSPAFMSATPGDKVNISCSASQGISNYLNWYQQKPGEAVKFIIYYTSSLHSGIPPRFSGSGYGTDYTLTINNIESEDAAYYYCQQYSKLPRTFGGGTKVEIK

SEQ ID NO: 3036 - protein, 107 aa, synthetic construct
DIVMTQTPLSSPVTLGQPASISCSASQGISNYLNWYQQRPGQPVKLLIYYTSSLHSGVPDRFSGSGAGTDYTLKISRVEAEDVGVYYCQQYSKLPRTFGGGTKVEIK

SEQ ID NO: 3037 - protein, 107 aa, synthetic construct
EIVMTQSPDFQSVTPKEKVTITCSASQGISNYLNWYQQKPDQSVKLLIYYTSSLHSGVPSRFSGSGSGTDYTLTINSLEAEDAATYYCQQYSKLPRTFGGGTKVEIK

SEQ ID NO: 3038 - protein, 107 aa, synthetic construct
EIVMTQTPLSLSITPGEQASISCSASQGISNYLNWYLQKARPVVKLLIYYTSSLHSGVPDRFSGSGSGTDYTLKISRVEAEDFGVYYCQQYSKLPRTFGGGTKVEIK

SEQ ID NO: 3039 - protein, 107 aa, synthetic construct
EIVMTQTPLSLSITPGEQASMSCSASQGISNYLNWYLQKARPVVKLLIYYTSSLHSGVPDRFSGSGSGTDYTLKISRVEAEDFGVYYCQQYSKLPRTFGGGTKVEIK

SEQ ID NO: 3040 - protein, 120 aa, synthetic construct
QVTLKESGPVLVKPTETLTLTCTVSGFSLTAYGVNWVRQPPGKALEWLGMIWGDGNTDYNSALKSRLTISKDNSKSQVVLTMTNMDPVDTATYYCARDRVTATLYAMDYWGQGTLVTVSS

SEQ ID NO: 3041 - protein, 120 aa, synthetic construct
QVTLKESGPALVKPTETLTLTCTVSGFSLTAYGVNWVRQPPGKALEWLGMIWGDGNTDYNSALKSRLIISKDNSKSQVVLTMTNMDPVDTATYYCARDRVTATLYAMDYWGQGTLVTVSS

SEQ ID NO: 3042 - protein, 120 aa, synthetic construct
QVTLKESGPALVKPTQTLTLTCTVSGFSLTAYGVNWVRQPPGKALEWLGMIWGDGNTDYNSALKSRLTISKDNSKSQVVLTMTNMDPVDTATYYCARDRVTATLYAMDYWGQGTLVTVSS

SEQ ID NO: 3043 - protein, 120 aa, synthetic construct
QVQLQESGPGLVKPSGTLSLTCAVSGFSLTAYGVNWVRQPPGKGLEWLGMIWGDGNTDYNSALKSRLTISKDNSKSQVSLKLSSVTAADTAVYYCARDRVTATLYAMDYWGQGTLVTVSS

SEQ ID NO: 3044 - protein, 120 aa, synthetic construct
QVTLKESGPTLVKPTQTLTLTCTVSGFSLTAYGVNWVRQPPGKALEWLGMIWGDGNTDYNSALKSRLTITKDNSKSQVVLTMTNMDPVDTATYYCARDRVTATLYAMDYWGQGTLVTVSS

SEQ ID NO: 3045 - protein, 120 aa, synthetic construct
QVTLKESGPALVKPTQTLTLTCTVSGFSLTAYGVNWVRQPPGKALEWLGMIWGDGNTDYNSALKSRLTITKDNSKSQVVLTMTNMDPVDTATYYCARDRVTATLYAMDYWGQGTLVTVSS

SEQ ID NO: 3046 - protein, 120 aa, synthetic construct
QVQLQESGPGLVKPSQTLSLTCTVSGFSLTAYGVNWVRQPPGKGLEWLGMIWGDGNTDYNSALKSRLTISKDNSKSQVSLKLSSVTAADTAVYYCARDRVTATLYAMDYWGQGTLVTVSS

SEQ ID NO: 3047 - protein, 120 aa, synthetic construct
QVQLQESGPGLVKPSETLSLTCTVSGFSLTAYGVNWVRQPPGKGLEWLGMIWGDGNTDYNSALKSRLTISKDNSKSQVSLKLSSVTAADTAVYYCARDRVTATLYAMDYWGQGTLVTVSS

SEQ ID NO: 3048 - protein, 120 aa, synthetic construct
QVQLQESGPGLVKPSQTLSLTCAVSGFSLTAYGVNWVRQPPGKGLEWLGMIWGDGNTDYNSALKSRLTISKDNSKSQVSLKLSSVTAADTAVYYCARDRVTATLYAMDYWGQGTLVTVSS

SEQ ID NO: 3049 - protein, 120 aa, synthetic construct
QVQLQESGPGLVKPSDTLSLTCTVSGFSLTAYGVNWVRQPPGKGLEWLGMIWGDGNTDYNSALKSRLTISKDNSKSQVSLKLSSVTAADTAVYYCARDRVTATLYAMDYWGQGTLVTVSS

SEQ ID NO: 3050 - protein, 120 aa, synthetic construct
QVQLQESGPGLVKPSQTLSLTCTVSGFSLTAYGVNWVRQHPGKGLEWLGMIWGDGNTDYNSALKSRLTISKDNSKSQVSLKLSSVTAADTAVYYCARDRVTATLYAMDYWGQGTLVTVSS

SEQ ID NO: 3051 - protein, 120 aa, synthetic construct
QVQLQESGPGLVKPSQTLSLTCTVSGFSLTAYGVNWVRQPAGKGLEWLGMIWGDGNTDYNSALKSRLTISKDNSKSQVSLKLSSVTAADTAVYYCARDRVTATLYAMDYWGQGTLVTVSS

SEQ ID NO: 3052 - protein, 120 aa, synthetic construct
QVQLQESGPGLVKPSQTLSLTCAVSGFSLTAYGVNWVRQPPGKGLEWLGMIWGDGNTDYNSALKSRLTISKDNSKSQVSLKLSSVTAVDTAVYYCARDRVTATLYAMDYWGQGTLVTVSS

SEQ ID NO: 3053 - protein, 120 aa, synthetic construct
QVQLQESGPGLVKPSETLSLTCTVSGFSLTAYGVNWVRQPPGKGLEWLGMIWGDGNTDYNSALKSRLTISKDNSKSHVSLKLSSVTAADTAVYYCARDRVTATLYAMDYWGQGTLVTVSS

SEQ ID NO: 3054 - protein, 120 aa, synthetic construct
QVQLQESGPGLVKPSETLSLTCAVSGFSLTAYGVNWVRQPPGKGLEWLGMIWGDGNTDYNSALKSRLTISKDNSKSQVSLKLSSVTAADTAVYYCARDRVTATLYAMDYWGQGTLVTVSS

SEQ ID NO: 3055 - protein, 120 aa, synthetic construct
QVQLQESGPGLVKPSQTLSLTCAVYGFSLTAYGVNWVRQPPGKGLEWLGMIWGDGNTDYNSALKSRLTISKDNSKSQVSLKLSSVTAADTAVYYCARDRVTATLYAMDYWGQGTLVTVSS

SEQ ID NO: 3056 - protein, 120 aa, synthetic construct
RVQLQESGPGLVKPSETLSLTCTVSGFSLTAYGVNWVRQPPGKGLEWLGMIWGDGNTDYNSALKSRLTISKDNSKSQVPLKLSSVTAADTAVYYCARDRVTATLYAMDYWGQGTLVTVSS

SEQ ID NO: 3057 - protein, 120 aa, synthetic construct
QVQLQESGPGLVKPSQTLSLTCTVSGFSLTAYGVNWVRQHPGKGLEWLGMIWGDGNTDYNSALKSLLTISKDNSKSQVSLKLSSVTAADTAVYYCARDRVTATLYAMDYWGQGTLVTVSS

SEQ ID NO: 3058 - protein, 120 aa, synthetic construct
QVQLQESGPGLVKPSDTLSLTCAVSGFSLTAYGVNWVRQPPGKGLEWLGMIWGDGNTDYNSALKSRLTISKDNSKSQVSLKLSSVTALDTAVYYCARDRVTATLYAMDYWGQGTLVTVSS

SEQ ID NO: 3059 - protein, 120 aa, synthetic construct
QVQLQESGPGLVKPSDTLSLTCAVSGFSLTAYGVNWVRQPPGKGLEWLGMIWGDGNTDYNSALKSRLTISKDNSKSQVSLKLSSVTAVDTAVYYCARDRVTATLYAMDYWGQGTLVTVSS

SEQ ID NO: 3060 - protein, 120 aa, synthetic construct
QVQLQESGSGLVKPSQTLSLTCAVSGFSLTAYGVNWVRQPPGKGLEWLGMIWGDGNTDYNSALKSRLTISKDNSKSQVSLKLSSVTAADTAVYYCARDRVTATLYAMDYWGQGTLVTVSS

SEQ ID NO: 3061 - protein, 120 aa, synthetic construct
EVQLVESGGGLVQPGRSLRLSCTVSGFSLTAYGVNWVRQAPGKGLEWLGMIWGDGNTDYNSALKSRLTISKDNSKSIVYLQMNSLKTEDTAVYYCARDRVTATLYAMDYWGQGTLVTVSS

SEQ ID NO: 3062 - protein, 120 aa, synthetic construct
EVQLVESGGGLVQPGPSLRLSCTVSGFSLTAYGVNWVRQAPGKGLEWLGMIWGDGNTDYNSALKSRLTISKDNSKSIVYLQMNSLKTEDTAVYYCARDRVTATLYAMDYWGQGTLVTVSS

SEQ ID NO: 3063 - protein, 120 aa, synthetic construct
QVQLQESGSGLVKPSQTLSLTCAVSGFSLTAYGVNWVRQSPGKGLEWLGMIWGDGNTDYNSALKSRLTISKDNSKSQVSLKLSSVTAADTAVYYCARDRVTATLYAMDYWGQGTLVTVSS

SEQ ID NO: 3064 - protein, 120 aa, synthetic construct
QVQLQESGPGLVKPSETLSLTCTVSGFSLTAYGVNWVRQPAGKGLEWLGMIWGDGNTDYNSALKSRLTISKDNSKSQVSLKLSSVTAADTAVYYCARDRVTATLYAMDYWGQGTLVTVSS

SEQ ID NO: 3065 - protein, 120 aa, synthetic construct
EVQLVESGGGLVKPGRSLRLSCTVSGFSLTAYGVNWVRQAPGKGLEWLGMIWGDGNTDYNSALKSRLTISKDNSKSIVYLQMNSLKTEDTAVYYCARDRVTATLYAMDYWGQGTLVTVSS

SEQ ID NO: 3066 - protein, 120 aa, synthetic construct
QVQLQESGPGLVKPSETLSLTCAVYGFSLTAYGVNWVRQPPGKGLEWLGMIWGDGNTDYNSALKSRLTISKDNSKSQVYLKLSSVTAADTAVYYCARDRVTATLYAMDYWGQGTLVTVSS

SEQ ID NO: 3067 - protein, 120 aa, synthetic construct
QVQLQESGPGLVKPSDTLSLTCAVSGFSLTAYGVNWVRQPPGKGLEWLGMIWGDGNTDYNSALKSRLTISKDNSKSQVSLKLSSVTAVDTGVYYCARDRVTATLYAMDYWGQGTLVTVSS

SEQ ID NO: 3068 - protein, 120 aa, synthetic construct
EVQLVESGGGLVQPGGSLRLSCAVSGFSLTAYGVNWVRQAPGKGLEWLGMIWGDGNTDYNSALKSRLTISKDNSKSSVYLQMNSLKTEDTAVYYCARDRVTATLYAMDYWGQGTLVTVSS

SEQ ID NO: 3069 - protein, 120 aa, synthetic construct
EVQLVESGGGLVKPGGSLRLSCAVSGFSLTAYGVNWVRQAPGKGLEWLGMIWGDGNTDYNSALKSRLTISKDNSKSTVYLQMNSLKTEDTAVYYCARDRVTATLYAMDYWGQGTLVTVSS

SEQ ID NO: 3070 - protein, 120 aa, synthetic construct
QVQLQQSGPGLVKPSQTLSLTCAVSGFSLTAYGVNWVRQSPSRGLEWLGMIWGDGNTDYNSALKSRLTINKDNSKSQVSLQLNSVTPEDTAVYYCARDRVTATLYAMDYWGQGTLVTVSS

SEQ ID NO: 3071 - protein, 120 aa, synthetic construct
QVQLVESGGGLVQPGGSLRLSCSVSGFSLTAYGVNWVRQAPGKGLEWLGMIWGDGNTDYNSALKSRLTISKDNSKSTVYLQMNSLRAEDTAVYYCARDRVTATLYAMDYWGQGTLVTVSS

SEQ ID NO: 3072 - protein, 120 aa, synthetic construct
QVQLQQWGAGLLKPSETLSLTCAVYGFSLTAYGVNWVRQPPGKGLEWLGMIWGDGNTDYNSALKSRLTISKDNSKSQVSLKLSSVTAADTAVYYCARDRVTATLYAMDYWGQGTLVTVSS

SEQ ID NO: 3073 - protein, 120 aa, synthetic construct
QVQLVESGGGVVQPGRSLRLSCAVSGFSLTAYGVNWVRQAPGKGLEWLGMIWGDGNTDYNSALKSRLTISKDNSTSTVFLQMNSLRAEDTAVYYCARDRVTATLYAMDYWGQGTLVTVSS

SEQ ID NO: 3074 - protein, 120 aa, synthetic construct
EVQLVESGGGLVQPGGSLRLSCAVSGFSLTAYGVNWVRQAPGKGLEWLGMIWGDGNTDYNSALKSRLTISKDNSKSTVYLQMNSLRAEDTAVYYCARDRVTATLYAMDYWGQGTLVTVSS

SEQ ID NO: 3075 - protein, 120 aa, synthetic construct
EVQLVESGGGLVQPGGSLRLSCAVSGFSLTAYGVNWVRQAPGKGLEWLGMIWGDGNTDYNSALKSRLTISKDNAKSSVYLQMNSLRDEDTAVYYCARDRVTATLYAMDYWGQGTLVTVSS

SEQ ID NO: 3076 - protein, 120 aa, synthetic construct
EVQLLESGGGLVQPGGSLRLSCAVSGFSLTAYGVNWVRQAPGKGLEWLGMIWGDGNTDYNSALKSRLTISKDNSKSTVYLQMNSLRAEDTAVYYCARDRVTATLYAMDYWGQGTLVTVSS

SEQ ID NO: 3077 - protein, 120 aa, synthetic construct
QVQLVESGGGLVKPGGSLRLSCAVSGFSLTAYGVNWVRQAPGKGLEWLGMIWGDGNTDYNSALKSRLTISKDNAKSSVYLQMNSLRAEDTAVYYCARDRVTATLYAMDYWGQGTLVTVSS

SEQ ID NO: 3078 - protein, 120 aa, synthetic construct
EVQLVESGGGLVQPGGSLKLSCAVSGFSLTAYGVNWVRQASGKGLEWLGMIWGDGNTDYNSALKSRLTISKDNSKSTVYLQMNSLKTEDTAVYYCARDRVTATLYAMDYWGQGTLVTVSS

SEQ ID NO: 3079 - protein, 120 aa, synthetic construct
QVQLLESGGGLVKPGGSLRLSCAVSGFSLTAYGVNWVRQAPGKGLEWLGMIWGDGNTDYNSALKSRLTISKDNAKSSVYLQMNSLRAEDTAVYYCARDRVTATLYAMDYWGQGTLVTVSS

SEQ ID NO: 3080 - protein, 120 aa, synthetic construct
QVQLVESGGGVVQPGRSLRLSCAVSGFSLTAYGVNWVRQAPGKGLEWLGMIWGDGNTDYNSALKSRLTISKDNSKSTVYLQMNSLRAEDTAVYYCARDRVTATLYAMDYWGQGTLVTVSS

SEQ ID NO: 3081 - protein, 120 aa, synthetic construct
QVQLVESGGGVVQPGRSLRLSCAVSGFSLTAYGVNWVRQAPGKGLEWLGMIWGDGNTDYNSALKSRLTISKDNSKSRVYLQMNSLRAEDTAVYYCARDRVTATLYAMDYWGQGTLVTVSS

SEQ ID NO: 3082 - protein, 120 aa, synthetic construct
QVQLVESGGGVVQPGRSLRLSCAVSGFSLTAYGVNWVRQAPGKGLEWLGMIWGDGNTDYNSALKSRLAISKDNSKSTVYLQMNSLRAEDTAVYYCARDRVTATLYAMDYWGQGTLVTVSS

SEQ ID NO: 3083 - protein, 120 aa, synthetic construct
QVQLVESGGGVVQPGGSLRLSCAVSGFSLTAYGVNWVRQAPGKGLEWLGMIWGDGNTDYNSALKSRLTISKDNSKSTVYLQMNSLRAEDTAVYYCARDRVTATLYAMDYWGQGTLVTVSS

SEQ ID NO: 3084 - protein, 120 aa, synthetic construct
EVQLVESGGGLVQPGGSLRLSCAVSGFSLTAYGVNWVRQAPGKGLEWLGMIWGDGNTDYNSALKSRLTISKDNAKSTVYLQMNSLRAEDTAVYYCARDRVTATLYAMDYWGQGTLVTVSS

SEQ ID NO: 3085 - protein, 120 aa, synthetic construct
EVQLVESGGGLVQPGGSLRLSCAVSGFSLTAYGVNWVRQAPGKGLEWLGMIWGDGNTDYNSALKSRLTISKDNAKSSVYLQMNSLRAEDTAVYYCARDRVTATLYAMDYWGQGTLVTVSS

SEQ ID NO: 3086 - protein, 120 aa, synthetic construct
EVQLVESGGVVVQPGGSLRLSCAVSGFSLTAYGVNWVRQAPGKGLEWLGMIWGDGNTDYNSALKSRLTISKDNSKSSVYLQMNSLRTEDTALYYCARDRVTATLYAMDYWGQGTLVTVSS

SEQ ID NO: 3087 - protein, 120 aa, synthetic construct
EVQLVESGGGLVQPGGSLRLSCAVSGFSLTAYGVNWVRQAPGKGLEWLGMIWGDGNTDYNSALKSRLTISKHNSKSTVYLQMNSLRAEDTAVYYCARDRVTATLYAMDYWGQGTLVTVSS

SEQ ID NO: 3088 - protein, 120 aa, synthetic construct
EVQLVESGGGLVKPGGSLRLSCAVSGFSLTAYGVNWVRQAPGKGLEWLGMIWGDGNTDYNSALKSRLTISKDNAKSSVYLQMNSLRAEDTAVYYCARDRVTATLYAMDYWGQGTLVTVSS

SEQ ID NO: 3089 - protein, 120 aa, synthetic construct
EVQLVESGGGLIQPGGSLRLSCAVSGFSLTAYGVNWVRQPPGKGLEWLGMIWGDGNTDYNSALKSRLTISKDNSKSTVYLQMNSLRAEDTAVYYCARDRVTATLYAMDYWGQGTLVTVSS

SEQ ID NO: 3090 - skipped

SEQ ID NO: 3091 - protein, 119 aa, synthetic construct
QVQLQQSGPELVKPGASVKISCKASGYAFSSSWMNWVKQRPGQGLEWIGRIYPGDGDTKYNGKFKGKATLTADKSSSTAYMHLSSLTSVDSAVYFCARRGTGGWYFDVWGAGTTVTVSS

SEQ ID NO: 3092 - protein, 111 aa, synthetic construct
DIVLTQSPASLAVSLGQRATISCRASESVDSSGNSFMHWYQQKPGQPPQLLIYRASNLESGIPARFSGSGSRTDFTLTINPVEADDVATFYCQQSFDDPFTFGSGTKLEIK

SEQ ID NO: 3093 - protein, 111 aa, synthetic construct
DIVLTQSPDSLAVSLGERATINCRASESVDSSGNSFMHWYQQKPGQPPQLLIYRASNLESGVPDRFSGSGSRTDFTLTISSLQAEDVAVYYCQQSFDDPFTFGQGTKLEIK

SEQ ID NO: 3094 - protein, 111 aa, synthetic construct
EIVLTQSPATLSLSPGERATLSCRASESVDSSGNSFMHWYQQKPGQAPQLLIYRASNLESGIPARFSGSGSRTDFTLTISSLEPEDFAVYYCQQSFDDPFTFGQGTKLEIK

SEQ ID NO: 3095 - protein, 111 aa, synthetic construct
EIVLTQSPATLSLSPGERATLSCRASESVDSSGNSFMHWYQQKPGQAPQLLIYRASNLESGIPARFSGSGSRTDFTLTISRLEPEDFAVYYCQQSFDDPFTFGQGTKLEIK

SEQ ID NO: 3096 - protein, 111 aa, synthetic construct
EIVLTQSPATLSLSPGERATLSCRASESVDSSGNSFMHWYQQKPGQAPQLLIYRASNLESGIPARFSGSGSRTDFTLTISSLQPEDFAVYYCQQSFDDPFTFGQGTKLEIK

SEQ ID NO: 3097 - protein, 111 aa, synthetic construct
DIQLTQSPSSLSASVGDRVTITCRASESVDSSGNSFMHWYQQKPGQAPQLLIYRASNLESGVPSRFSGSGSRTDFTLTISSLQPEDVATYYCQQSFDDPFTFGQGTKLEIK

SEQ ID NO: 3098 - protein, 111 aa, synthetic construct
EIVLTQSPATLSLSPGERATLSCRASESVDSSGNSFMHWYQQKPGQAPQLLIYRASNLESGIPARFSGSGPRTDFTLTISSLEPEDFAVYYCQQSFDDPFTFGQGTKLEIK

SEQ ID NO: 3099 - protein, 111 aa, synthetic construct
EIVLTQSPATLSLSPGERATLSCRASESVDSSGNSFMHWYQQKPGQAPQLLIYRASNLESGIPDRFSGSGSRTDFTLTISRLEPEDFAVYYCQQSFDDPFTFGQGTKLEIK

SEQ ID NO: 3100 - protein, 111 aa, synthetic construct
DIQLTQSPSSLSASVGDRVTITCRASESVDSSGNSFMHWYQQKPGKVPQLLIYRASNLESGVPSRFSGSGSRTDFTLTISSLQPEDVATYYCQQSFDDPFTFGQGTKLEIK

SEQ ID NO: 3101 - protein, 111 aa, synthetic construct
DIQLTQSPSSLSASVGDRVTITCRASESVDSSGNSFMHWYQQKPGKTPQLLIYRASNLESGIPSRFSGSGSRTDFTLTIRSLQPEDFATYYCQQSFDDPFTFGQGTKLEIK

SEQ ID NO: 3102 - protein, 111 aa, synthetic construct
EIVLTQSPGTLSLSPGERATLSCRASESVDSSGNSFMHWYQQKPGQAPQLLIYRASNLESGIPDRFSGSGSRTDFTLTISRLEPEDFAVYYCQQSFDDPFTFGQGTKLEIK

SEQ ID NO: 3103 - protein, 111 aa, synthetic construct
EIVLTQSPATLSLSPGERATLSCRASESVDSSGNSFMHWYQQKPGLAPQLLIYRASNLESGIPDRFSGSGSRTDFTLTISRLEPEDFAVYYCQQSFDDPFTFGQGTKLEIK

SEQ ID NO: 3104 - protein, 111 aa, synthetic construct
DIQLTQSPSSLSASVGDRVTITCRASESVDSSGNSFMHWYQQKPGKAPQLLIYRASNLESGVPSRFSGSGSRTDFTLTISSLQPEDFATYYCQQSFDDPFTFGQGTKLEIK

SEQ ID NO: 3105 - protein, 111 aa, synthetic construct
DIQLTQSPSSVSASVGDRVTITCRASESVDSSGNSFMHWYQQKPGKAPQLLIYRASNLESGVPSRFSGSGSRTDFTLTISSLQPEDFATYYCQQSFDDPFTFGQGTKLEIK

SEQ ID NO: 3106 - protein, 111 aa, synthetic construct
AIQLTQSPSSLSASVGDRVTITCRASESVDSSGNSFMHWYQQKPGKAPQLLIYRASNLESGVPSRFSGSGSRTDFTLTISSLQPEDFATYYCQQSFDDPFTFGQGTKLEIK

SEQ ID NO: 3107 - protein, 111 aa, synthetic construct
DIQLTQSPSFLSASVGDRVTITCRASESVDSSGNSFMHWYQQKPGKAPQLLIYRASNLESGVPSRFSGSGSRTEFTLTISSLQPEDFATYYCQQSFDDPFTFGQGTKLEIK

SEQ ID NO: 3108 - protein, 111 aa, synthetic construct
DIQLTQSPSSLSASVGDRVTITCRASESVDSSGNSFMHWYQQKPGKAPQLLIYRASNLESGVPSRFSGSGSRTDFTFTISSLQPEDIATYYCQQSFDDPFTFGQGTKLEIK

SEQ ID NO: 3109 - protein, 111 aa, synthetic construct
EIVLTQSPATLSVSPGERATLSCRASESVDSSGNSFMHWYQQKPGQAPQLLIYRASNLESGIPARFSGSGSRTEFTLTISILQSEDFAVYYCQQSFDDPFTFGQGTKLEIK

SEQ ID NO: 3110 - protein, 111 aa, synthetic construct
EIVLTQSPATLSVSPGERATLSCRASESVDSSGNSFMHWYQQKPGQAPQLLIYRASNLESGIPARFSGSGSRTEFTLTISSLQSEDFAVYYCQQSFDDPFTFGQGTKLEIK

SEQ ID NO: 3111 - protein, 111 aa, synthetic construct
AIRLTQSPFSLSASVGDRVTITCRASESVDSSGNSFMHWYQQKPAKAPQLFIYRASNLESGVPSRFSGSGSRTDFTLTISSLQPEDFATYYCQQSFDDPFTFGQGTKLEIK

SEQ ID NO: 3112 - protein, 111 aa, synthetic construct
DIQLTQSPSSLSASVGDRVTITCRASESVDSSGNSFMHWYQQKPGKAPQSLIYRASNLESGVPSRFSGSGSRTDFTLTISSLQPEDFATYYCQQSFDDPFTFGQGTKLEIK

SEQ ID NO: 3113 - protein, 111 aa, synthetic construct
DIQLTQSPSSLSASVGDRVTITCRASESVDSSGNSFMHWYQQKPGKAPQRLIYRASNLESGVPSRFSGSGSRTEFTLTISNLQPEDFATYYCQQSFDDPFTFGQGTKLEIK

SEQ ID NO: 3114 - protein, 111 aa, synthetic construct
DIQLTQSPSTLSASVGDRVTITCRASESVDSSGNSFMHWYQQKPGKAPQLLIYRASNLESGVPSRFSGSGSRTEFTLTISSLQPDDFATYYCQQSFDDPFTFGQGTKLEIK

SEQ ID NO: 3115 - protein, 111 aa, synthetic construct
EIVLTQSPDFQSVTPKEKVTITCRASESVDSSGNSFMHWYQQKPDQSPQLLIYRASNLESGVPSRFSGSGSRTDFTLTINSLEAEDAATYYCQQSFDDPFTFGQGTKLEIK

SEQ ID NO: 3116 - protein, 111 aa, synthetic construct
DIQLTQSPSSLSASVGDRVTITCRASESVDSSGNSFMHWYQQKPGKAPQSLIYRASNLESGVPSKFSGSGSRTDFTLTISSLQPEDFATYYCQQSFDDPFTFGQGTKLEIK

SEQ ID NO: 3117 - protein, 111 aa, synthetic construct
DIQLTQSPSSLSASVGDRVTITCRASESVDSSGNSFMHWYQQKPGKAPQRLIYRASNLESGVPSRFSGSGSRTEFTLTISSLQPEDFATYYCQQSFDDPFTFGQGTKLEIK

SEQ ID NO: 3118 - protein, 111 aa, synthetic construct
DIVLTQTPLSLSVTPGQPASISCRASESVDSSGNSFMHWYLQKPGQPPQLLIYRASNLESGVPDRFSGSGSRTDFTLKISRVEAEDVGVYYCQQSFDDPFTFGQGTKLEIK

SEQ ID NO: 3119 - protein, 111 aa, synthetic construct
DIQLTQSPSSLSASVGDRVTITCRASESVDSSGNSFMHWYQQKPEKAPQSLIYRASNLESGVPSRFSGSGSRTDFTLTISSLQPEDFATYYCQQSFDDPFTFGQGTKLEIK

SEQ ID NO: 3120 - protein, 111 aa, synthetic construct
EIVLTQSPPTLSLSPGERVTLSCRASESVDSSGNSFMHWYQQKPGQAPQLLIYRASNLESGIPARFSGSGSRTDFTLTISSLQPEDFAVYYCQQSFDDPFTFGQGTKLEIK

SEQ ID NO: 3121 - protein, 111 aa, synthetic construct
DIQLTQSPSAMSASVGDRVTITCRASESVDSSGNSFMHWYQQKPGKVPQRLIYRASNLESGVPSRFSGSGSRTEFTLTISSLQPEDFATYYCQQSFDDPFTFGQGTKLEIK

SEQ ID NO: 3122 - protein, 111 aa, synthetic construct
DIVLTQSPLSLPVTPGEPASISCRASESVDSSGNSFMHWYLQKPGQSPQLLIYRASNLESGVPDRFSGSGSRTDFTLKISRVEAEDVGVYYCQQSFDDPFTFGQGTKLEIK

SEQ ID NO: 3123 - protein, 111 aa, synthetic construct
DIVLTQTPLSLPVTPGEPASISCRASESVDSSGNSFMHWYLQKPGQSPQLLIYRASNLESGVPDRFSGSGSRTDFTLKISRVEAEDVGVYYCQQSFDDPFTFGQGTKLEIK

SEQ ID NO: 3124 - protein, 111 aa, synthetic construct
DIVLTQTPLSLSVTPGQPASISCRASESVDSSGNSFMHWYLQKPGQSPQLLIYRASNLESGVPDRFSGSGSRTDFTLKISRVEAEDVGVYYCQQSFDDPFTFGQGTKLEIK

SEQ ID NO: 3125 - protein, 111 aa, synthetic construct
EIVLTQSPPTLSLSPGERVTLSCRASESVDSSGNSFMHWYQQKPGQAPQLLIYRASNLESSIPARFSGSGSRTDFTLTISSLQPEDFAVYYCQQSFDDPFTFGQGTKLEIK

SEQ ID NO: 3126 - protein, 111 aa, synthetic construct
DIVLTQSPLSLPVTLGQPASISCRASESVDSSGNSFMHWYQQRPGQSPQRLIYRASNLESGVPDRFSGSGSRTDFTLKISRVEAEDVGVYYCQQSFDDPFTFGQGTKLEIK

SEQ ID NO: 3127 - protein, 111 aa, synthetic construct
DIVLTQTPLSSPVTLGQPASISCRASESVDSSGNSFMHWYQQRPGQPPQLLIYRASNLESGVPDRFSGSGARTDFTLKISRVEAEDVGVYYCQQSFDDPFTFGQGTKLEIK

SEQ ID NO: 3128 - protein, 111 aa, synthetic construct
DIVLTQSPAFLSVTPGEKVTITCRASESVDSSGNSFMHWYQQKPDQAPQLLIYRASNLESGVPSRFSGSGSRTDFTFTISSLEAEDAATYYCQQSFDDPFTFGQGTKLEIK

SEQ ID NO: 3129 - protein, 111 aa, synthetic construct
DIQLIQSPSFLSASVGDRVSIICRASESVDSSGNSFMHWYLQKPGKSPQLFIYRASNLESGVSSRFSGRGSRTDFTLTIISLKPEDFAAYYCQQSFDDPFTFGQGTKLEIK

SEQ ID NO: 3130 - protein, 111 aa, synthetic construct
EIVLTQTPLSLSITPGEQASISCRASESVDSSGNSFMHWYLQKARPVPQLLIYRASNLESGVPDRFSGSGSRTDFTLKISRVEAEDFGVYYCQQSFDDPFTFGQGTKLEIK

SEQ ID NO: 3131 - protein, 111 aa, synthetic construct
EIVLTQTPLSLSITPGEQASMSCRASESVDSSGNSFMHWYLQKARPVPQLLIYRASNLESGVPDRFSGSGSRTDFTLKISRVEAEDFGVYYCQQSFDDPFTFGQGTKLEIK

SEQ ID NO: 3132 - protein, 111 aa, synthetic construct
EITLTQSPAFMSATPGDKVNISCRASESVDSSGNSFMHWYQQKPGEAPQFIIYRASNLESGIPPRFSGSGYRTDFTLTINNIESEDAAYYYCQQSFDDPFTFGQGTKLEIK

SEQ ID NO: 3133 - protein, 119 aa, synthetic construct
QVQLVQSGAEVKKPGASVKVSCKASGYAFSSSWMNWVRQAPGQGLEWIGRIYPGDGDTKYNGKFKGRATLTADKSTSTAYMELSSLRSEDTAVYYCARRGTGGWYFDVWGQGTTVTVSS

SEQ ID NO: 3134 - protein, 119 aa, synthetic construct
QVQLVQSGAEVKKPGSSVKVSCKASGYAFSSSWMNWVRQAPGQGLEWIGRIYPGDGDTKYNGKFKGRATLTADKSTSTAYMELSSLRSEDTAVYYCARRGTGGWYFDVWGQGTTVTVSS

SEQ ID NO: 3135 - protein, 119 aa, synthetic construct
QVQLVQSGAEVKKPGASVKVSCKASGYAFSSSWMNWVRQAPGKGLEWIGRIYPGDGDTKYNGKFKGRATLTADKSTSTAYMELSSLRSEDTAVYYCARRGTGGWYFDVWGQGTTVTVSS

SEQ ID NO: 3136 - protein, 119 aa, synthetic construct
QVQLVQSGAEVKKPGASVKVSCKASGYAFSSSWMNWVRQAPGQELEWIGRIYPGDGDTKYNGKFKGRATLTADKSISTAYMELSSLRSEDTATYYCARRGTGGWYFDVWGQGTTVTVSS

SEQ ID NO: 3137 - protein, 119 aa, synthetic construct
EVQLVQSGAEVKKPGATVKISCKASGYAFSSSWMNWVQQAPGKGLEWIGRIYPGDGDTKYNGKFKGRATLTADKSTSTAYMELSSLRSEDTAVYYCARRGTGGWYFDVWGQGTTVTVSS

SEQ ID NO: 3138 - protein, 119 aa, synthetic construct
QVQLVQSGAEVKKTGSSVKVSCKASGYAFSSSWMNWVRQAPGQALEWIGRIYPGDGDTKYNGKFKGRATLTADKSMSTAYMELSSLRSEDTAMYYCARRGTGGWYFDVWGQGTTVTVSS

SEQ ID NO: 3139 - protein, 119 aa, synthetic construct
QVQLVQSGAEVKKPGASVKVSCKASGYAFSSSWMNWVRQAPGQRLEWIGRIYPGDGDTKYNGKFKGRATLTADKSASTAYMELSSLRSEDMAVYYCARRGTGGWYFDVWGQGTTVTVSS

SEQ ID NO: 3140 - protein, 119 aa, synthetic construct
QVQLVQSGAEVKKPGASVKVSCKASGYAFSSSWMNWVRQAPGQGLEWIGRIYPGDGDTKYNGKFKGRATLTADKSTSTAYMELRSLRSDDMAVYYCARRGTGGWYFDVWGQGTTVTVSS

SEQ ID NO: 3141 - protein, 119 aa, synthetic construct
QVQLVQSGAEVKKPGASVKVSCKASGYAFSSSWMNWVRQAPGQRLEWIGRIYPGDGDTKYNGKFKGRATLTADKSASTAYMELSSLRSEDTAVYYCARRGTGGWYFDVWGQGTTVTVSS

SEQ ID NO: 3142 - protein, 119 aa, synthetic construct
QVQLVQSGAEVKKPGASVKVSCKASGYAFSSSWMNWVRQAPGQGLEWIGRIYPGDGDTKYNGKFKGRATLTADKSTSTAYMELRSLRSDDTAVYYCARRGTGGWYFDVWGQGTTVTVSS

SEQ ID NO: 3143 - protein, 119 aa, synthetic construct
QVQLVQSGAEVKKPGASVKVSCKASGYAFSSSWMNWVRQAPGQGLEWIGRIYPGDGDTKYNGKFKGRATLTADKSISTAYMELSRLRSDDTAVYYCARRGTGGWYFDVWGQGTTVTVSS

SEQ ID NO: 3144 - protein, 119 aa, synthetic construct
QVQLVQSGAEVKKPGASVKVSCKASGYAFSSSWMNWVRQAPGQGLEWIGRIYPGDGDTKYNGKFKGRATLTADKSISTAYMELSRLRSDDTVVYYCARRGTGGWYFDVWGQGTTVTVSS

SEQ ID NO: 3145 - protein, 119 aa, synthetic construct
QVQLVQSGAEVKKPGASVKVSCKASGYAFSSSWMNWVRQAPGQGLEWIGRIYPGDGDTKYNGKFKGWATLTADKSISTAYMELSRLRSDDTAVYYCARRGTGGWYFDVWGQGTTVTVSS

SEQ ID NO: 3146 - protein, 119 aa, synthetic construct
QVQLVQSGAEVKKPGASVKVSCKASGYAFSSSWMNWVRQATGQGLEWIGRIYPGDGDTKYNGKFKGRATLTANKSISTAYMELSSLRSEDTAVYYCARRGTGGWYFDVWGQGTTVTVSS

SEQ ID NO: 3147 - protein, 119 aa, synthetic construct
QVQLVQSGSELKKPGASVKVSCKASGYAFSSSWMNWVRQAPGQGLEWIGRIYPGDGDTKYNGKFKGRAVLSADKSVSTAYLQISSLKAEDTAVYYCARRGTGGWYFDVWGQGTTVTVSS

SEQ ID NO: 3148 - protein, 119 aa, synthetic construct
QVQLVQSGPEVKKPGTSVKVSCKASGYAFSSSWMNWVRQARGQRLEWIGRIYPGDGDTKYNGKFKGRATLTADKSTSTAYMELSSLRSEDTAVYYCARRGTGGWYFDVWGQGTTVTVSS

SEQ ID NO: 3149 - protein, 119 aa, synthetic construct
EVQLVQSGAEVKKPGESLKISCKASGYAFSSSWMNWVRQMPGKGLEWIGRIYPGDGDTKYNGKFKGQATLSADKSISTAYLQWSSLKASDTAMYYCARRGTGGWYFDVWGQGTTVTVSS

SEQ ID NO: 3150 - protein, 119 aa, synthetic construct
QVQLVQSGSELKKPGASVKVSCKASGYAFSSSWMNWVRQAPGQGLEWIGRIYPGDGDTKYNGKFKGRAVLSADKSVSMAYLQISSLKAEDTAVYYCARRGTGGWYFDVWGQGTTVTVSS

SEQ ID NO: 3151 - protein, 119 aa, synthetic construct
QVQLVQSGHEVKQPGASVKVSCKASGYAFSSSWMNWVPQAPGQGLEWIGRIYPGDGDTKYNGKFKGRAVLSADKSASTAYLQISSLKAEDMAMYYCARRGTGGWYFDVWGQGTTVTVSS

SEQ ID NO: 3152 - protein, 119 aa, synthetic construct
EVQLVQSGAEVKKPGESLKISCKASGYAFSSSWMNWVRQMPGKGLEWIGRIYPGDGDTKYNGKFKGQATLSADKPISTAYLQWSSLKASDTAMYYCARRGTGGWYFDVWGQGTTVTVSS

SEQ ID NO: 3153 - protein, 119 aa, synthetic construct
EVQLVQSGAEVKKPGESLRISCKASGYAFSSSWMNWVRQMPGKGLEWIGRIYPGDGDTKYNGKFKGQATLSADKSISTAYLQWSSLKASDTAMYYCARRGTGGWYFDVWGQGTTVTVSS

SEQ ID NO: 3154 - protein, 119 aa, synthetic construct
EVQLVQSGAEVKKPGESLRISCKASGYAFSSSWMNWVRQMPGKGLEWIGRIYPGDGDTKYNGKFKGHATLSADKSISTAYLQWSSLKASDTAMYYCARRGTGGWYFDVWGQGTTVTVSS

SEQ ID NO: 3155 - protein, 119 aa, synthetic construct
QVQLVQSGAEVKKTGSSVKVSCKASGYAFSSSWMNWVRQAPRQALEWIGRIYPGDGDTKYNGKFKGRATLTADKSMSTAYMELSSLRSEDTAMYYCARRGTGGWYFDVWGQGTTVTVSS

SEQ ID NO: 3156 - protein, 119 aa, synthetic construct
EVQLVESGGGLVQPGRSLRLSCTASGYAFSSSWMNWVRQAPGKGLEWIGRIYPGDGDTKYNGKFKGRATLSADKSKSIAYLQMNSLKTEDTAVYYCARRGTGGWYFDVWGQGTTVTVSS

SEQ ID NO: 3157 - protein, 119 aa, synthetic construct
EVQLVESGGGLVQPGPSLRLSCTASGYAFSSSWMNWVRQAPGKGLEWIGRIYPGDGDTKYNGKFKGRATLSADKSKSIAYLQMNSLKTEDTAVYYCARRGTGGWYFDVWGQGTTVTVSS

SEQ ID NO: 3158 - protein, 119 aa, synthetic construct
QVQLQESGPGLVKPSQTLSLTCTASGYAFSSSWMNWVRQPPGKGLEWIGRIYPGDGDTKYNGKFKGRATLSADKSKSQASLKLSSVTAADTAVYYCARRGTGGWYFDVWGQGTTVTVSS

SEQ ID NO: 3159 - protein, 119 aa, synthetic construct
QVQLQESGPGLVKPSGTLSLTCAASGYAFSSSWMNWVRQPPGKGLEWIGRIYPGDGDTKYNGKFKGRATLSADKSKSQASLKLSSVTAADTAVYYCARRGTGGWYFDVWGQGTTVTVSS

SEQ ID NO: 3160 - protein, 119 aa, synthetic construct
EVQLVESGGGLVKPGRSLRLSCTASGYAFSSSWMNWVRQAPGKGLEWIGRIYPGDGDTKYNGKFKGRATLSADKSKSIAYLQMNSLKTEDTAVYYCARRGTGGWYFDVWGQGTTVTVSS

SEQ ID NO: 3161 - protein, 119 aa, synthetic construct
EVQLVESGGGLVQPGGSLKLSCAASGYAFSSSWMNWVRQASGKGLEWIGRIYPGDGDTKYNGKFKGRATLSADKSKSTAYLQMNSLKTEDTAVYYCARRGTGGWYFDVWGQGTTVTVSS

SEQ ID NO: 3162 - protein, 119 aa, synthetic construct
QVQLQESGPGLVKPSQTLSLTCAASGYAFSSSWMNWVRQPPGKGLEWIGRIYPGDGDTKYNGKFKGRATLSADKSKSQASLKLSSVTAADTAVYYCARRGTGGWYFDVWGQGTTVTVSS

SEQ ID NO: 3163 - protein, 119 aa, synthetic construct
EVQLVESGGGLVKPGGSLRLSCAASGYAFSSSWMNWVRQAPGKGLEWIGRIYPGDGDTKYNGKFKGRATLSADKSKSTAYLQMNSLKTEDTAVYYCARRGTGGWYFDVWGQGTTVTVSS

SEQ ID NO: 3164 - protein, 119 aa, synthetic construct
EVQLVESGGALVKPGGSLRLSCAASGYAFSSSWMNWVRQAPGKGLEWIGRIYPGDGDTKYNGKFKGRATLSADKSKSTAYLQMNSLKTEDTAVYYCARRGTGGWYFDVWGQGTTVTVSS

SEQ ID NO: 3165 - protein, 119 aa, synthetic construct
QVQLQESGPGLVKPSQTLSLTCAAYGYAFSSSWMNWVRQPPGKGLEWIGRIYPGDGDTKYNGKFKGRATLSADKSKSQASLKLSSVTAADTAVYYCARRGTGGWYFDVWGQGTTVTVSS

SEQ ID NO: 3166 - protein, 119 aa, synthetic construct
QVQLQESGSGLVKPSQTLSLTCAASGYAFSSSWMNWVRQPPGKGLEWIGRIYPGDGDTKYNGKFKGRATLSADKSKSQASLKLSSVTAADTAVYYCARRGTGGWYFDVWGQGTTVTVSS

SEQ ID NO: 3167 - protein, 119 aa, synthetic construct
EVQLVESGGGLVQPGGSLRLSCAASGYAFSSSWMNWVRQAPGKGLEWIGRIYPGDGDTKYNGKFKGRATLSADKSKSSAYLQMNSLKTEDTAVYYCARRGTGGWYFDVWGQGTTVTVSS

SEQ ID NO: 3168 - protein, 119 aa, synthetic construct
QVQLQESGPGLVKPSDTLSLTCTASGYAFSSSWMNWVRQPPGKGLEWIGRIYPGDGDTKYNGKFKGRATLSADKSKSQASLKLSSVTAADTAVYYCARRGTGGWYFDVWGQGTTVTVSS

SEQ ID NO: 3169 - protein, 119 aa, synthetic construct
QVQLQESGPGLVKPSQTLSLTCTASGYAFSSSWMNWVRQHPGKGLEWIGRIYPGDGDTKYNGKFKGRATLSADKSKSQASLKLSSVTAADTAVYYCARRGTGGWYFDVWGQGTTVTVSS

SEQ ID NO: 3170 - protein, 119 aa, synthetic construct
QVQLQESGPGLVKPSQTLSLTCTASGYAFSSSWMNWVRQHPGKGLEWIGRIYPGDGDTKYNGKFKGLATLSADKSKSQASLKLSSVTAADTAVYYCARRGTGGWYFDVWGQGTTVTVSS

SEQ ID NO: 3171 - protein, 119 aa, synthetic construct
QVQLVESGGGVVQPGRSLRLSCAASGYAFSSSWMNWVRQAPGKGLEWIGRIYPGDGDTKYNGKFKGRATLSADKSKSTAYLQMSSLRAEDTAVYYCARRGTGGWYFDVWGQGTTVTVSS

SEQ ID NO: 3172 - protein, 119 aa, synthetic construct
QVQLVESGGGLVKPGGSLRLSCAASGYAFSSSWMNWVRQAPGKGLEWIGRIYPGDGDTKYNGKFKGRATLSADKAKSSAYLQMNSLRAEDTAVYYCARRGTGGWYFDVWGQGTTVTVSS

SEQ ID NO: 3173 - protein, 119 aa, synthetic construct
QVQLVESGGGLVQPGGSLRLSCSASGYAFSSSWMNWVRQAPGKGLEWIGRIYPGDGDTKYNGKFKGRATLSADKSKSTAYLQMNSLRAEDTAVYYCARRGTGGWYFDVWGQGTTVTVSS

SEQ ID NO: 3174 - protein, 119 aa, synthetic construct
QVQLLESGGGLVKPGGSLRLSCAASGYAFSSSWMNWVRQAPGKGLEWIGRIYPGDGDTKYNGKFKGRATLSADKAKSSAYLQMNSLRAEDTAVYYCARRGTGGWYFDVWGQGTTVTVSS

SEQ ID NO: 3175 - protein, 119 aa, synthetic construct
EVQLVESGGGLVQPGGSLRLSCSASGYAFSSSWMNWVRQAPGKGLEWIGRIYPGDGDTKYNGKFKGRATLSADKSKSTAYLQMSSLRAEDTAVYYCARRGTGGWYFDVWGQGTTVTVSS

SEQ ID NO: 3176 - protein, 119 aa, synthetic construct
QVQLQESGPGLVKPSDTLSLTCAASGYAFSSSWMNWVRQPPGKGLEWIGRIYPGDGDTKYNGKFKGRATLSADKSKSQASLKLSSVTAVDTAVYYCARRGTGGWYFDVWGQGTTVTVSS

SEQ ID NO: 3177 - protein, 119 aa, synthetic construct
QVQLQESGPGLVKPSQTLSLTCAASGYAFSSSWMNWVRQPPGKGLEWIGRIYPGDGDTKYNGKFKGRATLSADKSKSQASLKLSSVTAVDTAVYYCARRGTGGWYFDVWGQGTTVTVSS

SEQ ID NO: 3178 - protein, 119 aa, synthetic construct
EVQLVESGGGLVQPGGSLRLSCSASGYAFSSSWMNWVRQAPGKGLEWIGRIYPGDGDTKYNGKFKGRATLSADKSKSTAYVQMSSLRAEDTAVYYCARRGTGGWYFDVWGQGTTVTVSS

SEQ ID NO: 3179 - protein, 119 aa, synthetic construct
QVQLVDSGGGVVQPGRSLRLSCAASGYAFSSSWMNWVRQAPGKGLEWIGRIYPGDGDTKYNGKFKGRATLSADKSKSTAYLQMNSLRAEDTAVYYCARRGTGGWYFDVWGQGTTVTVSS

SEQ ID NO: 3180 - protein, 119 aa, synthetic construct
QVQLVESGGGVVQPGRSLRLSCAASGYAFSSSWMNWVRQAPGKGLEWIGRIYPGDGDTKYNGKFKGRATLSADKSKSTAYLQMNSLRAEGTAVYYCARRGTGGWYFDVWGQGTTVTVSS

SEQ ID NO: 3181 - protein, 119 aa, synthetic construct
QVQLVESGGGVVQPGRSLRLSCAASGYAFSSSWMNWVRQAPGKGLEWIGRIYPGDGDTKYNGKFKGRATLSADKSKSTAYLQMNSLRAEDTAVYYCARRGTGGWYFDVWGQGTTVTVSS

SEQ ID NO: 3182 - protein, 119 aa, synthetic construct
EVQLVESGGGLVQPGGSLRLSCAASGYAFSSSWMNWVRQAPGKGLEWIGRIYPGDGDTKYNGKFKGRATLSADKSKSTAYLQMNSLRAEDTAVYYCARRGTGGWYFDVWGQGTTVTVSS

SEQ ID NO: 3183 - protein, 117 aa, synthetic construct
EVQLVESGGDLVKPGGSLKLSCAVSGFTFRSYGMSWVRQTPDKRLEWVALISSGGSYTYYTDSVKGRFTISRDNAKNTLYLQMSSLKSEDTAIYYCSRHGGNFFDYWGQGTTLTVSS

SEQ ID NO: 3184 - protein, 107 aa, synthetic construct
QIVLTQSPSIMSASPGEKVTMTCSVSSSVSYMHWYQQKSGTSPKRWIYDTSKLASGVPARFSGSGSGTSYSLTISSMEAEDAATYYCQQWSSNPQYTFGGGTKLEIK

SEQ ID NO: 3185 - protein, 107 aa, synthetic construct
DIVLTQSPAFLSVTPGEKVTITCSVSSSVSYMHWYQQKPDQAPKLLIYDTSKLASGVPSRFSGSGSGTDYTFTISSLEAEDAATYYCQQWSSNPQYTFGQGTKLEIK

SEQ ID NO: 3186 - protein, 107 aa, synthetic construct
AIQLTQSPSSLSASVGDRVTITCSVSSSVSYMHWYQQKPGKAPKLLIYDTSKLASGVPSRFSGSGSGTDYTLTISSLQPEDFATYYCQQWSSNPQYTFGQGTKLEIK

SEQ ID NO: 3187 - protein, 107 aa, synthetic construct
DIQLTQSPSFLSASVGDRVTITCSVSSSVSYMHWYQQKPGKAPKLLIYDTSKLASGVPSRFSGSGSGTEYTLTISSLQPEDFATYYCQQWSSNPQYTFGQGTKLEIK

SEQ ID NO: 3188 - protein, 107 aa, synthetic construct
DIQLTQSPSSLSASVGDRVTITCSVSSSVSYMHWYQQKPGKAPKLLIYDTSKLASGVPSRFSGSGSGTDYTLTISSLQPEDFATYYCQQWSSNPQYTFGQGTKLEIK

SEQ ID NO: 3189 - protein, 107 aa, synthetic construct
DIQLTQSPSSVSASVGDRVTITCSVSSSVSYMHWYQQKPGKAPKLLIYDTSKLASGVPSRFSGSGSGTDYTLTISSLQPEDFATYYCQQWSSNPQYTFGQGTKLEIK

SEQ ID NO: 3190 - protein, 107 aa, synthetic construct
DIQLTQSPSSLSASVGDRVTITCSVSSSVSYMHWYQQKPGKVPKLLIYDTSKLASGVPSRFSGSGSGTDYTLTISSLQPEDVATYYCQQWSSNPQYTFGQGTKLEIK

SEQ ID NO: 3191 - protein, 107 aa, synthetic construct
DIQLTQSPSSLSASVGDRVTITCSVSSSVSYMHWYQQKPGQAPKLLIYDTSKLASGVPSRFSGSGSGTDYTLTISSLQPEDVATYYCQQWSSNPQYTFGQGTKLEIK

SEQ ID NO: 3192 - protein, 107 aa, synthetic construct
EIVLTQSPDFQSVTPKEKVTITCSVSSSVSYMHWYQQKPDQSPKLLIYDTSKLASGVPSRFSGSGSGTDYTLTINSLEAEDAATYYCQQWSSNPQYTFGQGTKLEIK

SEQ ID NO: 3193 - protein, 107 aa, synthetic construct
AIRLTQSPFSLSASVGDRVTITCSVSSSVSYMHWYQQKPAKAPKLFIYDTSKLASGVPSRFSGSGSGTDYTLTISSLQPEDFATYYCQQWSSNPQYTFGQGTKLEIK

SEQ ID NO: 3194 - protein, 107 aa, synthetic construct
DIQLTQSPSSLSASVGDRVTITCSVSSSVSYMHWYQQKPGKAPKLLIYDTSKLASGVPSRFSGSGSGTDYTFTISSLQPEDIATYYCQQWSSNPQYTFGQGTKLEIK

SEQ ID NO: 3195 - protein, 107 aa, synthetic construct
EIVLTQSPATLSLSPGERATLSCSVSSSVSYMHWYQQKPGQAPKLLIYDTSKLASGIPARFSGSGSGTDYTLTISSLEPEDFAVYYCQQWSSNPQYTFGQGTKLEIK

SEQ ID NO: 3196 - protein, 107 aa, synthetic construct
DIQLTQSPSTLSASVGDRVTITCSVSSSVSYMHWYQQKPGKAPKLLIYDTSKLASGVPSRFSGSGSGTEYTLTISSLQPDDFATYYCQQWSSNPQYTFGQGTKLEIK

SEQ ID NO: 3197 - protein, 107 aa, synthetic construct
DIQLTQSPSSLSASVGDRVTITCSVSSSVSYMHWYQQKPGKTPKLLIYDTSKLASGIPSRFSGSGSGTDYTLTIRSLQPEDFATYYCQQWSSNPQYTFGQGTKLEIK

SEQ ID NO: 3198 - protein, 107 aa, synthetic construct
EIVLTQSPPTLSLSPGERVTLSCSVSSSVSYMHWYQQKPGQAPKLLIYDTSKLASGIPARFSGSGSGTDYTLTISSLQPEDFAVYYCQQWSSNPQYTFGQGTKLEIK

SEQ ID NO: 3199 - protein, 107 aa, synthetic construct
DIQLTQSPSSLSASVGDRVTITCSVSSSVSYMHWYQQKPGKAPKRLIYDTSKLASGVPSRFSGSGSGTEYTLTISSLQPEDFATYYCQQWSSNPQYTFGQGTKLEIK

SEQ ID NO: 3200 - protein, 107 aa, synthetic construct
EIVLTQSPATLSLSPGERATLSCSVSSSVSYMHWYQQKPGQAPKLLIYDTSKLASGIPARFSGSGPGTDYTLTISSLEPEDFAVYYCQQWSSNPQYTFGQGTKLEIK

SEQ ID NO: 3201 - protein, 107 aa, synthetic construct
EIVLTQSPATLSLSPGERATLSCSVSSSVSYMHWYQQKPGQAPKLLIYDTSKLASGIPARFSGSGSGTDYTLTISRLEPEDFAVYYCQQWSSNPQYTFGQGTKLEIK

SEQ ID NO: 3202 - protein, 107 aa, synthetic construct
EIVLTQSPATLSLSPGERATLSCSVSSSVSYMHWYQQKPGQAPKLLIYDTSKLASGIPARFSGSGSGTDYTLTISSLQPEDFAVYYCQQWSSNPQYTFGQGTKLEIK

SEQ ID NO: 3203 - protein, 107 aa, synthetic construct
EIVLTQSPATLSVSPGERATLSCSVSSSVSYMHWYQQKPGQAPKLLIYDTSKLASGIPARFSGSGSGTEYTLTISSLQSEDFAVYYCQQWSSNPQYTFGQGTKLEIK

SEQ ID NO: 3204 - protein, 107 aa, synthetic construct
EIVLTQSPATLSVSPGERATLSCSVSSSVSYMHWYQQKPGQAPKLLIYDTSKLASGIPARFSGSGSGTEYTLTISILQSEDFAVYYCQQWSSNPQYTFGQGTKLEIK

SEQ ID NO: 3205 - protein, 107 aa, synthetic construct
EIVLTQSPPTLSLSPGERVTLSCSVSSSVSYMHWYQQKPGQAPKLLIYDTSKLASSIPARFSGSGSGTDYTLTISSLQPEDFAVYYCQQWSSNPQYTFGQGTKLEIK

SEQ ID NO: 3206 - protein, 107 aa, synthetic construct
DIQLTQSPSSLSASVGDRVTITCSVSSSVSYMHWYQQKPGKAPKSLIYDTSKLASGVPSRFSGSGSGTDYTLTISSLQPEDFATYYCQQWSSNPQYTFGQGTKLEIK

SEQ ID NO: 3207 - protein, 107 aa, synthetic construct
DIQLTQSPSSLSASVGDRVTITCSVSSSVSYMHWYQQKPGKAPKRLIYDTSKLASGVPSRFSGSGSGTEYTLTISNLQPEDFATYYCQQWSSNPQYTFGQGTKLEIK

SEQ ID NO: 3208 - protein, 107 aa, synthetic construct
DIQLTQSPSAMSASVGDRVTITCSVSSSVSYMHWYQQKPGKVPKRLIYDTSKLASGVPSRFSGSGSGTEYTLTISSLQPEDFATYYCQQWSSNPQYTFGQGTKLEIK

SEQ ID NO: 3209 - protein, 107 aa, synthetic construct
EIVLTQSPATLSLSPGERATLSCSVSSSVSYMHWYQQKPGQAPKLLIYDTSKLASGIPDRFSGSGSGTDYTLTISRLEPEDFAVYYCQQWSSNPQYTFGQGTKLEIK

SEQ ID NO: 3210 - protein, 107 aa, synthetic construct
EIVLTQSPATLSLSPGERATLSCSVSSSVSYMHWYQQKPGLAPKLLIYDTSKLASGIPDRFSGSGSGTDYTLTISRLEPEDFAVYYCQQWSSNPQYTFGQGTKLEIK

SEQ ID NO: 3211 - protein, 107 aa, synthetic construct
EIVLTQSPGTLSLSPGERATLSCSVSSSVSYMHWYQQKPGQAPKLLIYDTSKLASGIPDRFSGSGSGTDYTLTISRLEPEDFAVYYCQQWSSNPQYTFGQGTKLEIK

SEQ ID NO: 3212 - protein, 107 aa, synthetic construct
DIQLTQSPSSLSASVGDRVTITCSVSSSVSYMHWYQQKPGKAPKSLIYDTSKLASGVPSKFSGSGSGTDYTLTISSLQPEDFATYYCQQWSSNPQYTFGQGTKLEIK

SEQ ID NO: 3213 - protein, 107 aa, synthetic construct
DIQLTQSPSSLSASVGDRVTITCSVSSSVSYMHWYQQKPEKAPKSLIYDTSKLASGVPSRFSGSGSGTDYTLTISSLQPEDFATYYCQQWSSNPQYTFGQGTKLEIK

SEQ ID NO: 3214 - protein, 107 aa, synthetic construct
DIVLTQSPDSLAVSLGERATINCSVSSSVSYMHWYQQKPGQPPKLLIYDTSKLASGVPDRFSGSGSGTDYTLTISSLQAEDVAVYYCQQWSSNPQYTFGQGTKLEIK

SEQ ID NO: 3215 - protein, 107 aa, synthetic construct
EIVLTQTPLSLSITPGEQASMSCSVSSSVSYMHWYLQKARPVPKLLIYDTSKLASGVPDRFSGSGSGTDYTLKISRVEAEDFGVYYCQQWSSNPQYTFGQGTKLEIK

SEQ ID NO: 3216 - protein, 107 aa, synthetic construct
EIVLTQTPLSLSITPGEQASISCSVSSSVSYMHWYLQKARPVPKLLIYDTSKLASGVPDRFSGSGSGTDYTLKISRVEAEDFGVYYCQQWSSNPQYTFGQGTKLEIK

SEQ ID NO: 3217 - protein, 107 aa, synthetic construct
DIVLTQSPLSLPVTPGEPASISCSVSSSVSYMHWYLQKPGQSPKLLIYDTSKLASGVPDRFSGSGSGTDYTLKISRVEAEDVGVYYCQQWSSNPQYTFGQGTKLEIK

SEQ ID NO: 3218 - protein, 107 aa, synthetic construct
DIVLTQSPLSLPVTLGQPASISCSVSSSVSYMHWYQQRPGQSPKRLIYDTSKLASGVPDRFSGSGSGTDYTLKISRVEAEDVGVYYCQQWSSNPQYTFGQGTKLEIK

SEQ ID NO: 3219 - protein, 107 aa, synthetic construct
DIVLTQTPLSLPVTPGEPASISCSVSSSVSYMHWYLQKPGQSPKLLIYDTSKLASGVPDRFSGSGSGTDYTLKISRVEAEDVGVYYCQQWSSNPQYTFGQGTKLEIK

SEQ ID NO: 3220 - protein, 107 aa, synthetic construct
DIVLTQTPLSLSVTPGQPASISCSVSSSVSYMHWYLQKPGQSPKLLIYDTSKLASGVPDRFSGSGSGTDYTLKISRVEAEDVGVYYCQQWSSNPQYTFGQGTKLEIK

SEQ ID NO: 3221 - protein, 107 aa, synthetic construct
DIVLTQTPLSLSVTPGQPASISCSVSSSVSYMHWYLQKPGQPPKLLIYDTSKLASGVPDRFSGSGSGTDYTLKISRVEAEDVGVYYCQQWSSNPQYTFGQGTKLEIK

SEQ ID NO: 3222 - protein, 107 aa, synthetic construct
DIQLIQSPSFLSASVGDRVSIICSVSSSVSYMHWYLQKPGKSPKLFIYDTSKLASGVSSRFSGRGSGTDYTLTIISLKPEDFAAYYCQQWSSNPQYTFGQGTKLEIK

SEQ ID NO: 3223 - protein, 107 aa, synthetic construct
DIVLTQTPLSSPVTLGQPASISCSVSSSVSYMHWYQQRPGQPPKLLIYDTSKLASGVPDRFSGSGAGTDYTLKISRVEAEDVGVYYCQQWSSNPQYTFGQGTKLEIK

SEQ ID NO: 3224 - protein, 107 aa, synthetic construct
EITLTQSPAFMSATPGDKVNISCSVSSSVSYMHWYQQKPGEAPKFIIYDTSKLASGIPPRFSGSGYGTDYTLTINNIESEDAAYYYCQQWSSNPQYTFGQGTKLEIK

SEQ ID NO: 3225 - protein, 117 aa, synthetic construct
EVQLVESGGGLVKPGGSLRLSCAVSGFTFRSYGMSWVRQAPGKGLEWVALISSGGSYTYYTDSVKGRFTISRDNSKNTLYLQMNSLKTEDTAVYYCSRHGGNFFDYWGQGTTVTVSS

SEQ ID NO: 3226 - protein, 117 aa, synthetic construct
EVQLVESGGALVKPGGSLRLSCAVSGFTFRSYGMSWVRQAPGKGLEWVALISSGGSYTYYTDSVKGRFTISRDNSKNTLYLQMNSLKTEDTAVYYCSRHGGNFFDYWGQGTTVTVSS

SEQ ID NO: 3227 - protein, 117 aa, synthetic construct
EVQLVESGGGLVQPGGSLRLSCAVSGFTFRSYGMSWVRQAPGKGLEWVALISSGGSYTYYTDSVKGRFTISRDNAKNTLYLQMNSLRAEDTAVYYCSRHGGNFFDYWGQGTTVTVSS

SEQ ID NO: 3228 - protein, 117 aa, synthetic construct
EVQLVESGGGLVQPGGSLRLSCAVSGFTFRSYGMSWVRQAPGKGLEWVALISSGGSYTYYTDSVKGRFTISRDNAKNSLYLQMNSLRAEDTAVYYCSRHGGNFFDYWGQGTTVTVSS

SEQ ID NO: 3229 - protein, 117 aa, synthetic construct
EVQLVESGGGLVQPGGSLRLSCAVSGFTFRSYGMSWVRQAPGKGLEWVALISSGGSYTYYTDSVKGRFTISRDNSKNSLYLQMNSLKTEDTAVYYCSRHGGNFFDYWGQGTTVTVSS

SEQ ID NO: 3230 - protein, 117 aa, synthetic construct
EVQLVESGGGLVQPGGSLRLSCAVSGFTFRSYGMSWVRQAPGKGLEWVALISSGGSYTYYTDSVKGRFTISRDNAKNSLYLQMNSLRAEDMAVYYCSRHGGNFFDYWGQGTTVTVSS

SEQ ID NO: 3231 - protein, 117 aa, synthetic construct
EVQLVESGGGLVQPGGSLRLSCAVSGFTFRSYGMSWVRQAPGKGLEWVALISSGGSYTYYTDSVKGQFTISRDNAKNTLYLQMNSLRAEDMAVYYCSRHGGNFFDYWGQGTTVTVSS

SEQ ID NO: 3232 - protein, 117 aa, synthetic construct
EVQLVESGGGLVKPGRSLRLSCTVSGFTFRSYGMSWVRQAPGKGLEWVALISSGGSYTYYTDSVKGRFTISRDNSKNILYLQMNSLKTEDTAVYYCSRHGGNFFDYWGQGTTVTVSS

SEQ ID NO: 3233 - protein, 117 aa, synthetic construct
EVQLVESGGGLVKPGGSLRLSCAVSGFTFRSYGMSWVRQAPGKGLEWVALISSGGSYTYYTDSVKGRFTISRDNAKNSLYLQMNSLRAEDTAVYYCSRHGGNFFDYWGQGTTVTVSS

SEQ ID NO: 3234 - protein, 117 aa, synthetic construct
EVQLVESGGGLVQPGGSLKLSCAVSGFTFRSYGMSWVRQASGKGLEWVALISSGGSYTYYTDSVKGRFTISRDNSKNTLYLQMNSLKTEDTAVYYCSRHGGNFFDYWGQGTTVTVSS

SEQ ID NO: 3235 - protein, 117 aa, synthetic construct
QVQLVESGGGVVQPGGSLRLSCAVSGFTFRSYGMSWVRQAPGKGLEWVALISSGGSYTYYTDSVKGRFTISRDNSKNTLYLQMNSLRAEDTAVYYCSRHGGNFFDYWGQGTTVTVSS

SEQ ID NO: 3236 - protein, 117 aa, synthetic construct
QVQLVESGGGVVQPGRSLRLSCAVSGFTFRSYGMSWVRQAPGKGLEWVALISSGGSYTYYTDSVKGRFTISRDNSKNTLYLQMSSLRAEDTAVYYCSRHGGNFFDYWGQGTTVTVSS

SEQ ID NO: 3237 - protein, 117 aa, synthetic construct
EVQLVESGGGLVQPGGSLRLSCPVSGFTFRSYGMSWVRQAPGKGLEWVALISSGGSYTYYTDSVKGRFTISRDNANNSLYLQMNSLRAEDTAVYYCSRHGGNFFDYWGQGTTVTVSS

SEQ ID NO: 3238 - protein, 117 aa, synthetic construct
EVQLVESGGGLVQPGRSLRLSCTVSGFTFRSYGMSWVRQAPGKGLEWVALISSGGSYTYYTDSVKGRFTISRDNSKNILYLQMNSLKTEDTAVYYCSRHGGNFFDYWGQGTTVTVSS

SEQ ID NO: 3239 - protein, 117 aa, synthetic construct
EVQLVESGGGLVQPGPSLRLSCTVSGFTFRSYGMSWVRQAPGKGLEWVALISSGGSYTYYTDSVKGRFTISRDNSKNILYLQMNSLKTEDTAVYYCSRHGGNFFDYWGQGTTVTVSS

SEQ ID NO: 3240 - protein, 117 aa, synthetic construct
EVQLVESGGGLVQPGGSLRLSCAVSGFTFRSYGMSWVRQAPGKGLEWVALISSGGSYTYYTDSVKGRFTISRDNSKNTLYLQMNSLRAEDTAVYYCSRHGGNFFDYWGQGTTVTVSS

SEQ ID NO: 3241 - protein, 117 aa, synthetic construct
EVQLVESGGGLVQPGGSLRLSCAVSGFTFRSYGMSWVRQAPGKGLEWVALISSGGSYTYYTDSVKGRFTISRDNAKNSLYLQMNSLRDEDTAVYYCSRHGGNFFDYWGQGTTVTVSS

SEQ ID NO: 3242 - protein, 117 aa, synthetic construct
QVQLVESGGGLVKPGGSLRLSCAVSGFTFRSYGMSWVRQAPGKGLEWVALISSGGSYTYYTDSVKGRFTISRDNAKNSLYLQMNSLRAEDTAVYYCSRHGGNFFDYWGQGTTVTVSS

SEQ ID NO: 3243 - protein, 117 aa, synthetic construct
QVQLVESGGGVVQPGRSLRLSCAVSGFTFRSYGMSWVRQAPGKGLEWVALISSGGSYTYYTDSVKGRFTISRDNSKNTLYLQMNSLRAEDTAVYYCSRHGGNFFDYWGQGTTVTVSS

SEQ ID NO: 3244 - protein, 117 aa, synthetic construct
EVQLLESGGGLVQPGGSLRLSCAVSGFTFRSYGMSWVRQAPGKGLEWVALISSGGSYTYYTDSVKGRFTISRDNSKNTLYLQMNSLRAEDTAVYYCSRHGGNFFDYWGQGTTVTVSS

SEQ ID NO: 3245 - protein, 117 aa, synthetic construct
EVQLVESGGGLVQPGGSLRLSCAVSGFTFRSYGMSWVRQAPGKGLEWVALISSGGSYTYYTDSVKGRFTISRHNSKNTLYLQMNSLRAEDTAVYYCSRHGGNFFDYWGQGTTVTVSS

SEQ ID NO: 3246 - protein, 117 aa, synthetic construct
EVQLVESGGGLIQPGGSLRLSCAVSGFTFRSYGMSWVRQPPGKGLEWVALISSGGSYTYYTDSVKGRFTISRDNSKNTLYLQMNSLRAEDTAVYYCSRHGGNFFDYWGQGTTVTVSS

SEQ ID NO: 3247 - protein, 117 aa, synthetic construct
EVQLVESGGGLIQPGGSLRLSCAVSGFTFRSYGMSWVRQAPGKGLEWVALISSGGSYTYYTDSVKGRFTISRDNSKNTLYLQMNSLRAEDTAVYYCSRHGGNFFDYWGQGTTVTVSS

SEQ ID NO: 3248 - protein, 117 aa, synthetic construct
EVQLVESGGGLVQPGRSLRLSCAVSGFTFRSYGMSWVRQAPGKGLEWVALISSGGSYTYYTDSVKGRFTISRDNAKNSLYLQMNSLRAEDTALYYCSRHGGNFFDYWGQGTTVTVSS

SEQ ID NO: 3249 - protein, 117 aa, synthetic construct
QVQLVESGGGVVQPGRSLRLSCAVSGFTFRSYGMSWVRQAPGKGLEWVALISSGGSYTYYTDSVKGRFTISRDNSKNRLYLQMNSLRAEDTAVYYCSRHGGNFFDYWGQGTTVTVSS

SEQ ID NO: 3250 - protein, 117 aa, synthetic construct
QVQLVESGGGVVQPGRSLRLSCAVSGFTFRSYGMSWVRQAPGKGLEWVALISSGGSYTYYTDSVKGRFTISRDNSKNTLYLQMNSLRAEGTAVYYCSRHGGNFFDYWGQGTTVTVSS

SEQ ID NO: 3251 - protein, 117 aa, synthetic construct
QVQLVESGGGVVQPGRSLRLSCAVSGFTFRSYGMSWVRQAPGKGLEWVALISSGGSYTYYTDSVKGRFAISRDNSKNTLYLQMNSLRAEDTAVYYCSRHGGNFFDYWGQGTTVTVSS

SEQ ID NO: 3252 - protein, 117 aa, synthetic construct
QVQLVDSGGGVVQPGRSLRLSCAVSGFTFRSYGMSWVRQAPGKGLEWVALISSGGSYTYYTDSVKGRFTISRDNSKNTLYLQMNSLRAEDTAVYYCSRHGGNFFDYWGQGTTVTVSS

SEQ ID NO: 3253 - protein, 117 aa, synthetic construct
EVQLVESGGGVVRPGGSLRLSCAVSGFTFRSYGMSWVRQAPGKGLEWVALISSGGSYTYYTDSVKGRFTISRDNAKNSLYLQMNSLRAEDTALYHCSRHGGNFFDYWGQGTTVTVSS

SEQ ID NO: 3254 - protein, 117 aa, synthetic construct
EVQLVESGGVVVQPGGSLRLSCAVSGFTFRSYGMSWVRQAPGKGLEWVALISSGGSYTYYTDSVKGRFTISRDNSKNSLYLQMNSLRAEDTALYYCSRHGGNFFDYWGQGTTVTVSS

SEQ ID NO: 3255 - protein, 117 aa, synthetic construct
EVQLVESGGGVVQPGGSLRLSCAVSGFTFRSYGMSWVRQAPGKGLEWVALISSGGSYTYYTDSVKGRFTISRDNSKNSLYLQMNSLRTEDTALYYCSRHGGNFFDYWGQGTTVTVSS

SEQ ID NO: 3256 - protein, 117 aa, synthetic construct
EVQLVESGGVVVQPGGSLRLSCAVSGFTFRSYGMSWVRQAPGKGLEWVALISSGGSYTYYTDSVKGRFTISRDNSKNSLYLQMNSLRTEDTALYYCSRHGGNFFDYWGQGTTVTVSS

SEQ ID NO: 3257 - protein, 117 aa, synthetic construct
EVQLVETGGGLIQPGGSLRLSCAVSGFTFRSYGMSWVRQAPGKGLEWVALISSGGSYTYYTDSVKGRFTISRDNSKNTLYLQMNSLRAEDTAVYYCSRHGGNFFDYWGQGTTVTVSS

SEQ ID NO: 3258 - protein, 117 aa, synthetic construct
EVQLVESGGGLVQPGGSLRLSCAVSGFTFRSYGMSWVRQATGKGLEWVALISSGGSYTYYTDSVKGRFTISRENAKNSLYLQMNSLRAGDTAVYYCSRHGGNFFDYWGQGTTVTVSS

SEQ ID NO: 3259 - protein, 117 aa, synthetic construct
EVQLVESRGVLVQPGGSLRLSCAVSGFTFRSYGMSWVRQAPGKGLEWVALISSGGSYTYYTDSVKGRFTISRDNSKNTLHLQMNSLRAEDTAVYYCSRHGGNFFDYWGQGTTVTVSS

SEQ ID NO: 3260 - protein, 117 aa, synthetic construct
EVQLVESGGGLVQPGRSLRLSCAVSGFTFRSYGMSWVRQAPGKGLEWVALISSGGSYTYYTDSVKGRFTISRDNAKNSLYLQMNSLRAEDMALYYCSRHGGNFFDYWGQGTTVTVSS

SEQ ID NO: 3261 - protein, 117 aa, synthetic construct
QVQLVESGGGLVQPGGSLRLSCSVSGFTFRSYGMSWVRQAPGKGLEWVALISSGGSYTYYTDSVKGRFTISRDNSKNTLYLQMNSLRAEDTAVYYCSRHGGNFFDYWGQGTTVTVSS

SEQ ID NO: 3262 - protein, 117 aa, synthetic construct
EVQLVESGGGLVQPGGSLRLSCSVSGFTFRSYGMSWVRQAPGKGLEWVALISSGGSYTYYTDSVKGRFTISRDNSKNTLYLQMSSLRAEDTAVYYCSRHGGNFFDYWGQGTTVTVSS

SEQ ID NO: 3263 - protein, 117 aa, synthetic construct
QVQLVESGGGVVQPGRSLRLSCAVSGFTFRSYGMSWVRQAPGKGLEWVALISSGGSYTYYTDSVKGRFTISRDNSTNTLFLQMNSLRAEDTAVYYCSRHGGNFFDYWGQGTTVTVSS

SEQ ID NO: 3264 - protein, 117 aa, synthetic construct
QVQLLESGGGLVKPGGSLRLSCAVSGFTFRSYGMSWVRQAPGKGLEWVALISSGGSYTYYTDSVKGRFTISRDNAKNSLYLQMNSLRAEDTAVYYCSRHGGNFFDYWGQGTTVTVSS

SEQ ID NO: 3265 - protein, 117 aa, synthetic construct
EVQLVESGEGLVQPGGSLRLSCAVSGFTFRSYGMSWVRQAPGKGLEWVALISSGGSYTYYTDSVKGRFTISRDNSKNTLYLQMGSLRAEDMAVYYCSRHGGNFFDYWGQGTTVTVSS

SEQ ID NO: 3266 - protein, 117 aa, synthetic construct
EVQLVESGGGLVQPGGSLRLSCAVSGFTFRSYGMSWVRQAPGKGLEWVALISSGGSYTYYTDSVKGRFTISRDNSKNTLYLQMGSLRAEDMAVYYCSRHGGNFFDYWGQGTTVTVSS

SEQ ID NO: 3267 - protein, 117 aa, synthetic construct
EVQLVESGGGLVQPGGSLRLSCSVSGFTFRSYGMSWVRQAPGKGLEWVALISSGGSYTYYTDSVKGRFTISRDNSKNTLYVQMSSLRAEDTAVYYCSRHGGNFFDYWGQGTTVTVSS

SEQ ID NO: 3268 - protein, 117 aa, synthetic construct
EVQLVESGGGLVQPGGSLRLSCAVSGFTFRSYGMSWVRQAPGKGLEWVALISSGGSYTYYTDSVKGRFIISRDNSRNSLYLQKNRRRAEDMAVYYCSRHGGNFFDYWGQGTTVTVSS

SEQ ID NO: 3269 - protein, 117 aa, synthetic construct
EVQLVESGGGLVQPGGSLRLSCAVSGFTFRSYGMSWVHQAPGKGLEWVALISSGGSYTYYTDSVKGRFIISRDNSRNTLYLQTNSLRAEDTAVYYCSRHGGNFFDYWGQGTTVTVSS

SEQ ID NO: 3270 - protein, 117 aa, synthetic construct
EVHLVESGGGLVQPGGALRLSCAVSGFTFRSYGMSWVRQATGKGLEWVALISSGGSYTYYTDSVKGRFTISRENAKNSLYLQMNSLRAGDTAVYYCSRHGGNFFDYWGQGTTVTVSS

SEQ ID NO: 3271 - protein, 117 aa, synthetic construct
EVQLVESGGGLVQPRGSLRLSCAVSGFTFRSYGMSWVRQAPGKGLEWVALISSGGSYTYYTDSVKGRFTISRDNSKNTLYLQMNNLRAEGTAVYYCSRHGGNFFDYWGQGTTVTVSS

SEQ ID NO: 3272 - protein, 117 aa, synthetic construct
EVQLVESGGGLVQPRGSLRLSCAVSGFTFRSYGMSWVRQAPGKGLEWVALISSGGSYTYYTDSVKGRFTISRDNSKNTLYLQMNNLRAEGTAAYYCSRHGGNFFDYWGQGTTVTVSS

SEQ ID NO: 3273 - protein, 117 aa, synthetic construct
QVQLVQSGAEVKKPGASVKVSCKVSGFTFRSYGMSWVRQAPGKGLEWVALISSGGSYTYYTDSVKGRFTITRDNSTNTLYMELSSLRSEDTAVYYCSRHGGNFFDYWGQGTTVTVSS

SEQ ID NO: 3274 - protein, 117 aa, synthetic construct
QVQLVQSGSELKKPGASVKVSCKVSGFTFRSYGMSWVRQAPGQGLEWVALISSGGSYTYYTDSVKGRFVISRDNSVNTLYLQISSLKAEDTAVYYCSRHGGNFFDYWGQGTTVTVSS

SEQ ID NO: 3275 - protein, 470 aa, synthetic construct
METDTLLLWVLLLWVPGSTGQVQLVESGGGVVQPGRSLRLSCAASGIDFSRYWMSWVRQAPGKGLEWVGEINPDSSTINYAPSLKDRFTISRDNSKNTLYLQMSSLRAEDTAVYYCASLYYDYGDAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3276 - protein, 503 aa, synthetic construct
METDTLLLWVLLLWVPGSTGQVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIKGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 3277 - protein, 234 aa, synthetic construct; identical to SEQ ID NO: 1007

SEQ ID NO: 3278 - protein, 469 aa, synthetic construct
METDTLLLWVLLLWVPGSTGQVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3279 - protein, 234 aa, synthetic construct
METDTLLLWVLLLWVPGSTGDIQMTQSPSSLSASVGDRVTITCKASQNVGINVVWHQQKPGKVPKALIYSSSHRYSGVPSRFSGSGSGTDFTLTISSLQPEDVATYFCQQFKSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3280 - protein, 247 aa, synthetic construct
METDTLLLWVLLLWVPGSTGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 3281 - protein, 520 aa, synthetic construct
METDTLLLWVLLLWVPGSTGEVQLVESGGGLVQPGGSLRLSCAASGFTFSNFGMHWVRQAPGKGLEWVSYISSGSSTIYYADTLKGRFTISRDNSKNTLYLQMNSLRAEDTAVYYCARRGEGAMDYWGQGTTVTVSSGGGGSGGGGSGGGGSGGGGSDNQLTQSPSFLSASVGDRVTITCRASSSVNYIYWYQQKPGKAPKLLIYYTSNLAPGVPSRFSGSGSGNEYTLTISSLQPEDFATYYCQQFTSSPFTFGQGTKLEIKGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGLNDIFEAQKIEWHE

SEQ ID NO: 3282-3283 - skipped (2 entries)

SEQ ID NO: 3284 - protein, 469 aa, synthetic construct
METDTLLLWVLLLWVPGSTGQVQLQQSGPELVKPGASVKISCKASGYAFSSSWMNWVKQRPGQGLEWIGRIYPGDGDTKYNGKFKGKATLTADKSSSTAYMHLSSLTSVDSAVYFCARRGTGGWYFDVWGAGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 3285 - protein, 238 aa, synthetic construct
METDTLLLWVLLLWVPGSTGDIVLTQSPASLAVSLGQRATISCRASESVDSSGNSFMHWYQQKPGQPPQLLIYRASNLESGIPARFSGSGSRTDFTLTINPVEADDVATFYCQQSFDDPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3286 - protein, 739 aa, synthetic construct
METDTLLLWVLLLWVPGSTGQVQLVESGGGVVQPGRSLRLSCAASGIDFSRYWMSWVRQAPGKGLEWVGEINPDSSTINYAPSLKDRFTISRDNSKNTLYLQMSSLRAEDTAVYYCASLYYDYGDAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFTFSTYAMNWVRQAPGKGLEWVSRIRSKYNNYATYYADSVKDRFTISRDDSKNTLYLQMNSLKTEDTAVYYCVRHGNFGNSYVSWFAYWGQGTLVTVSSGGGGSGGGGSGGGGSGGGGSQAVVTQEPSLTVSPGGTVTLTCRSSTGAVTTSNYANWVQQKPGQAPRGLIGGTNKRAPWTPARFSGSLLGGKAALTLSGAQPEDEAEYYCALWYSNLWVFGGGTKLTVL

SEQ ID NO: 3287 - protein, 247 aa, synthetic construct
METDTLLLWVLLLWVPGSTGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 3288 - skipped

SEQ ID NO: 3289 - protein, 107 aa, synthetic construct; identical to SEQ ID NO: 230
  VARIANT 30: note = G, E, A or D
  VARIANT 32: note = N or D
  VARIANT 34: note = R or K
  VARIANT 39: note = Y or H
  VARIANT 60: note = K or S

SEQ ID NO: 3290 - protein, 119 aa, synthetic construct; identical to SEQ ID NO: 231
  VARIANT 27: note = H, T, G or Y
  VARIANT 28: note = D, T or S
  VARIANT 30: note = H, R, D, K or T
  VARIANT 31: note = L, D, K, T or N
  VARIANT 32: note = W, F, T, I, Y or G
  VARIANT 50: note = R or W
  VARIANT 51: note = V, I or F
  VARIANT 52: note = F, S or Y
  VARIANT 53: note = A or P
  VARIANT 57: note = N or S
  VARIANT 58: note = T, V, Y or I
  VARIANT 59: note = K or R
  VARIANT 98: note = G or V
  VARIANT 100: note = Y or I
  VARIANT 103: note = Y or A
  VARIANT 104: note = D or G

SEQ ID NO: 3291 - protein, 238 aa, synthetic construct
DLKVEMMAGGTQITPLNDNVTIFCNIFYSQPLNITSMGITWFWKSLTFDKEVKVFEFFGDHQEAFRPGAIVSPWRLKSGDASLRLPGIQLEEAGEYRCEVVVTPLKAQGTVQLEVVASPASRLLLDQVGMKENEDKYMCESSGFYPEAINITWEKQTQKFPHPIEISEDVITGPTIKNMDGTFNVTSCLKLNSSQEDPGTVYQCVVRHASLHTPLRSNFTLTAARHSLSETEKTDNFS

SEQ ID NO: 3292 - protein, 284 aa, synthetic construct
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHW

SEQ ID NO: 3293 - protein, 287 aa, synthetic construct
AEPHSLRYNLMVLSQDESVQSGFLAEGHLDGQPFLRYDRQKRRAKPQGQWAEDVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKVLVLQSQRTD

SEQ ID NO: 3294 - protein, 191 aa, synthetic construct
GWVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPG

SEQ ID NO: 3295 - protein, 329 aa, synthetic construct
QDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPNTAGAGATGG

SEQ ID NO: 3296 - protein, 323 aa, synthetic construct
WPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRN

SEQ ID NO: 3297 - protein, 330 aa, synthetic construct
QNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQKDTAVEGEEIEVNCTAMASKPATTIRWFKGNTELKGKSEVEEWSDMYTVTSQLMLKVHKEDDGVPVICQVEHPAVTGNLQTQRYLEVQYKPQVHIQMTYPLQGLTREGDALELTCEAIGKPQPVMVTWVRVDDEMPQHAVLSGPNLFINNLNKTDNGTYRCEASNIVGKAHSDYMLYVYDPPTTIPPPTTTTTTTTTTTTTILTIITDSRAGEEGSIRAVDH

SEQ ID NO: 3298 - protein, 155 aa, synthetic construct
QRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 3299 - protein, 294 aa, synthetic construct
WTYHYSEKPMNWQRARRFCRDNYTDLVAIQNKAEIEYLEKTLPFSRSYYWIGIRKIGGIWTWVGTNKSLTEEAENWGDGEPNNKKNKEDCVEIYIKRNKDAGKWNDDACHKLKAALCYTASCQPWSCSGHGECVEIINNYTCNCDVGYYGPQCQFVIQCEPLEAPELGTMDCTHPLGNFSFSSQCAFSCSEGTNLTGIEETTCGPFGNWSSPEPTCQVIQCEPLSAPDLGIMNCSHPLASFSFTSACTFICSEGTELIGKKKTICESSGIWSNPSPICQKLDKSFSMIKEGDYN

SEQ ID NO: 3300 - protein, 243 aa, synthetic construct
RYLQVSQQLQQTNRVLEVTNSSLRQQLRLKITQLGQSAEDLQGSRRELAQSQEALQVEQRAHQAAEGQLQACQADRQKTKETLQSEEQQRRALEQKLSNMENRLKPFFTCGSADTCCPSGWIMHQKSCFYISLTSKNWQESQKQCETLSSKLATFSEIYPQSHSYYFLNSLLPNGGSGNSYWTGLSSNKDWKLTDDTQRTRTYAQSSKCNKVHKTWSWWTLESESCRSSLPYICEMTAFRFPD

SEQ ID NO: 3301 - protein, 124 aa, synthetic construct
KLTRDSQSLCPYDWIGFQNKCYYFSKEEGDWNSSKYNCSTQHADLTIIDNIEEMNFLRRYKCSSDHWIGLKMAKNRTGQWVDGATFTKSFGMRGSEGCAYLSDDGAATARCYTERKWICRKRIH

SEQ ID NO: 3302 - protein, 194 aa, synthetic construct
QGHLVHMTVVSGSNVTLNISESLPENYKQLTWFYTFDQKIVEWDSRKSKYFESKFKGRVRLDPQSGALYISKVQKEDNSTYIMRVLKKTGNEQEWKIKLQVLDPVPKPVIKIEKIEDMDDNCYLKLSCVIPGESVNYTWYGDKRPFPKELQNSVLETTLMPHNYSRCYTCQVSNSVSSKNGTVCLSPPCTLARS

SEQ ID NO: 3303 - protein, 133 aa, synthetic construct
QVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 3304 - protein, 149 aa, synthetic construct
MQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 3305 - protein, 205 aa, synthetic construct
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 3306 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 3307 - protein, 5 aa, synthetic construct
GGGGS

SEQ ID NO: 3308 - protein, 10 aa, synthetic construct
GGGGSGGGGS

SEQ ID NO: 3309 - protein, 15 aa, synthetic construct
GGGGSGGGGSGGGGS

SEQ ID NO: 3310 - protein, 17 aa, synthetic construct
DVPSGPGGGGGSGGGGS

SEQ ID NO: 3311 - protein, 455 aa, synthetic construct; identical to SEQ ID NO: 3306

SEQ ID NO: 3312 - protein, 413 aa, synthetic construct
FRGPLLPNRPFTTVWNANTQWCLERHGVDVDVSVFDVVANPGQTFRGPDMTIFYSSQGTYPYYTPTGEPVFGGLPQNASLIAHLARTFQDILAAIPAPDFSGLAVIDWEAWRPRWAFNWDTKDIYRQRSRALVQAQHPDWPAPQVEAVAQDQFQGAARAWMAGTLQLGRALRPRGLWGFYGFPDCYNYDFLSPNYTGQCPSGIRAQNDQLGWLWGQSRALYPSIYMPAVLEGTGKSQMYVQHRVAEAFRVAVAAGDPNLPVLPYVQIFYDTTNHFLPLDELEHSLGESAAQGAAGVVLWVSWENTRTKESCQAIKEYMDTTLGPFILNVTSGALLCSQALCSGHGRCVRRTSHPKALLLLNPASFSIQLTPGGGPLSLRGALSLEDQAQMAVEFKCRCYPGWQAPWCERKSMW

SEQ ID NO: 3313 - protein, 551 aa, synthetic construct
YNFFPRKPKWDKNQITYRIIGYTPDLDPETVDDAFARAFQVWSDVTPLRFSRIHDGEADIMINFGRWEHGDGYPFDGKDGLLAHAFAPGTGVGGDSHFDDDELWTLGEGQVVRVKYGNADGEYCKFPFLFNGKEYNSCTDTGRSDGFLWCSTTYNFEKDGKYGFCPHEALFTMGGNAEGQPCKFPFRFQGTSYDSCTTEGRTDGYRWCGTTEDYDRDKKYGFCPETAMSTVGGNSEGAPCVFPFTFLGNKYESCTSAGRSDGKMWCATTANYDDDRKWGFCPDQGYSLFLVAAHEFGHAMGLEHSQDPGALMAPIYTYTKNFRLSQDDIKGIQELYGASPDIDLGTGPTPTLGPVTPEICKQDIVFDGIAQIRGEIFFFKDRFIWRTVTPRDKPMGPLLVATFWPELPEKIDAVYEAPQEEKAVFFAGNEYWIYSASTLERGYPKPLTSLGLPPDVQRVDAAFNWSKNKKTYIFAGDKFWRYNEVKKKMDPGFPKLIADAWNAIPDNLDAVVDLQGGGHSYFFKGAYYLKLENQSLKSVKFGSIKSDWLGC

SEQ ID NO: 3314 - protein, 14 aa, synthetic construct
AEAAAKEAAAKAAA

SEQ ID NO: 3315 - protein, 19 aa, synthetic construct
AEAAAKEAAAKEAAAKAAA

SEQ ID NO: 3316 - protein, 24 aa, synthetic construct
AEAAAKEAAAKEAAAKEAAAKAAA

SEQ ID NO: 3317 - protein, 29 aa, synthetic construct
AEAAAKEAAAKEAAAKEAAAKEAAAKAAA

SEQ ID NO: 3318 - protein, 449 aa, synthetic construct
QVQLVQSGAEVKKPGASVKVSCKASGFNIKSAYMHWVRQAPGQEPGCMGRIDPATGKTKYAPKFQARVTMTADTSINTAYTELSSLRSEDTATYYCARSLNWDYGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 3319 - protein, 218 aa, synthetic construct
EIVLTQSPATLSLSPGERATLSCRASKSVSILGTHLIHWYQQKPGLAPRLLIYAASNLESGIPDRFSGSGSETDFTLTISRLEPEDFAVYFCQQSIEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3320 - skipped

SEQ ID NO: 3321 - protein, 10 aa, synthetic construct
GFNIKSAYMH

SEQ ID NO: 3322 - protein, 17 aa, synthetic construct
RIDPATGKTKYAPKFQA

SEQ ID NO: 3323 - protein, 10 aa, synthetic construct
SLNWDYGLDY

SEQ ID NO: 3324 - protein, 15 aa, synthetic construct
RASKSVSILGTHLIH

SEQ ID NO: 3325 - protein, 7 aa, synthetic construct
AASNLES

SEQ ID NO: 3326 - protein, 9 aa, synthetic construct
QQSIEDPWT

SEQ ID NO: 3327 - skipped

SEQ ID NO: 3328 - protein, 449 aa, synthetic construct
QVQLVQSGAEVKKPGASVKVSCKASGFNIKSAYMHWVRQAPGQGLEWMGRIDPATGKTKYAPKFQARVTMTADTSTNTAYMELSSLRSEDTAVYYCARSLNWDYGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 3329 - protein, 218 aa, synthetic construct
EIVLTQSPGTLSLSPGERATLSCRASKSVSILGTHLIHWYQQKPGQAPRLLIYAASNLESGIPDRFSGSGSETDFTLTISRLEPEDFAVYFCQQSIEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3330-3337 - skipped (8 entries)

SEQ ID NO: 3338 - protein, 448 aa, synthetic construct
QVQLVQSGAEVKKPGASVKVSCKASGYTFTDYNIHWVRQAPGQGLEWMGYINPYNGRTGYNQKFKARVTMTVDKSTSTAYMELSSLRSEDTAVYYCARWDGSSYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 3339 - protein, 213 aa, synthetic construct
EIVLTQSPATLSVSPGERATLSCSASSSVSYMHWYQQKPGQAPRPLIYEISKLASGIPARFSGSGSGTEYTLTISSLQSEDFAVYYCQQWNYPLLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3340 - protein, 370 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3341 - protein, 10 aa, synthetic construct
GYTFTDYNIH

SEQ ID NO: 3342 - protein, 17 aa, synthetic construct
YINPYNGRTGYNQKFKA

SEQ ID NO: 3343 - protein, 9 aa, synthetic construct
WDGSSYFDY

SEQ ID NO: 3344 - protein, 10 aa, synthetic construct
SASSSVSYMH

SEQ ID NO: 3345 - protein, 7 aa, synthetic construct
EISKLAS

SEQ ID NO: 3346 - protein, 9 aa, synthetic construct
QQWNYPLLT

SEQ ID NO: 3347 - skipped

SEQ ID NO: 3348 - protein, 447 aa, synthetic construct
QVQLVQSGAEVKKPGSSVKVSCKASGHDFRLTYIHWVRQAPGQGLEWMGRVSAGSGNVKYNEKFKGRVTITADTSTSTAYMELSSLRSEDTAVYYCAVSYYSYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 3349 - protein, 214 aa, synthetic construct
DIQMTQSPSFLSASVGDRVTITCKASQNVADRVVWHQQKPGKAPKALIYSSSHRYKGVPSRFSGSGSGTEFTLTISSLQPEDFATYFCQQFKSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3350 - protein, 368 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 3351 - protein, 10 aa, synthetic construct
GHDFRLTYIH

SEQ ID NO: 3352 - protein, 17 aa, synthetic construct
RVSAGSGNVKYNEKFKG

SEQ ID NO: 3353 - protein, 10 aa, synthetic construct
SYYSYDVLDY

SEQ ID NO: 3354 - protein, 11 aa, synthetic construct
KASQNVADRVV

SEQ ID NO: 3355 - protein, 7 aa, synthetic construct
SSSHRYK

SEQ ID NO: 3356 - protein, 9 aa, synthetic construct
QQFKSYPLT

SEQ ID NO: 3357 - skipped

SEQ ID NO: 3358 - protein, 451 aa, synthetic construct
QIQLQQSGPEVVKPGASVQISCKASGYTFSGSWMNWVKQRPGKGLEWIGRIYPGDGDTDYSGKFKGRATLTADKSSSTAYMRLSSLTSEDSAVYFCARSGYFNYVPVFDYWGQGTTLSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 3359 - protein, 212 aa, synthetic construct
QIVLTQSPAIMSASLGEEIALTCSANSTVGYIHWYQQKSGTSPKLLIYTTSNLASGVPSRFSGSGSGTFYSLTISSVEAEDAADYFCHQWSFYPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3360-3367 - skipped (8 entries)

SEQ ID NO: 3368 - protein, 450 aa, synthetic construct
NVQLQESGPGLVKPSQSLSLTCSVAGYSITSGYFWNWIRQFPGNKLEWMGYISYDGSNNYNPSLKNRISITRDTSKNQFFLKLNSVTTEDTATYYCASPSPGTGYAVDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 3369 - protein, 219 aa, synthetic construct
NVVMTQTPLSLPVSLGDQASISCRSSQSLVHSNGNTYLHWYLQKPGQSPKFLIYKVSNRFSGVPDRFSGGGSGTEFTLKISRVEAEDLGVYFCSQSTHVPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3370 - skipped

SEQ ID NO: 3371 - protein, 9 aa, synthetic construct
SITSGYFWN

SEQ ID NO: 3372 - protein, 16 aa, synthetic construct
YISYDGSNNYNPSLKN

SEQ ID NO: 3373 - protein, 11 aa, synthetic construct
PSPGTGYAVDY

SEQ ID NO: 3374 - protein, 16 aa, synthetic construct
RSSQSLVHSNGNTYLH

SEQ ID NO: 3375 - protein, 7 aa, synthetic construct
KVSNRFS

SEQ ID NO: 3376 - protein, 9 aa, synthetic construct
SQSTHVPFT

SEQ ID NO: 3377 - skipped

SEQ ID NO: 3378 - protein, 451 aa, synthetic construct
QVQLQQPGAELVRPGASVKLSCKASGYTFTNYWMNWVKQRPGQGLEWIGMIHPSDSATRLNQKFKDKATLTVDKSSSTAYMQLSSPTSEDSAVYYCARWAYYGNLYAIDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVCVLPPPEEEMTKKQVTLSCAVTDFMPEDIYVEWTNNGKTELNYKNTEPVLDSDGSYFMVSKLRVEKKNWVERNSYSCSVVHEGLHNHHTTKSFSRTPGK

SEQ ID NO: 3379 - protein, 218 aa, synthetic construct
DIVLTQSPASLAVSLGQRATISCKASQSVDYDGYSYMHWFQQKPGQPPKLLIYAASNLESGIPARFIGSGSGTDFTLNIHPVEEEDAATYYCQQSNEDPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3380 - protein, 371 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPCEEEMTKKQVTLWCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 3381 - protein, 10 aa, synthetic construct
GYTFTNYWMN

SEQ ID NO: 3382 - protein, 17 aa, synthetic construct
MIHPSDSATRLNQKFKD

SEQ ID NO: 3383 - protein, 12 aa, synthetic construct
WAYYGNLYAIDY

SEQ ID NO: 3384 - protein, 15 aa, synthetic construct
KASQSVDYDGYSYMH

SEQ ID NO: 3385 - skipped

SEQ ID NO: 3386 - protein, 9 aa, synthetic construct
QQSNEDPWT

SEQ ID NO: 3387 - skipped

SEQ ID NO: 3388 - protein, 451 aa, synthetic construct
EVQLVESGGGLVQPGASLKLSCVASGFTFSDYWMSWVRQTPGKTMEWIGNIKYDGSHTNYAPSLKNRFTISRDNAKSTLFLQMSNVRSEDTATYYCTRESHYGYIYPFDYWGQGVMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVCVLPPPEEEMTKKQVTLSCAVTDFMPEDIYVEWTNNGKTELNYKNTEPVLDSDGSYFMVSKLRVEKKNWVERNSYSCSVVHEGLHNHHTTKSFSRTPGK

SEQ ID NO: 3389 - protein, 214 aa, synthetic construct
DIQMTQSPSSMSVSLGDTVTITCRASQDVGIYVNWFQQKPGKSPRRMIYRATNLADGVPSRFSGSRSGSDYSLTISSLESEDVADYHCLQYDESPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3390 - protein, 371 aa, synthetic construct; identical to SEQ ID NO: 3380

SEQ ID NO: 3391 - protein, 10 aa, synthetic construct
GFTFSDYWMS

SEQ ID NO: 3392 - protein, 17 aa, synthetic construct
NIKYDGSHTNYAPSLKN

SEQ ID NO: 3393 - protein, 12 aa, synthetic construct
ESHYGYIYPFDY

SEQ ID NO: 3394 - protein, 11 aa, synthetic construct
RASQDVGIYVN

SEQ ID NO: 3395 - protein, 7 aa, synthetic construct
RATNLAD

SEQ ID NO: 3396 - protein, 9 aa, synthetic construct
LQYDESPFT

SEQ ID NO: 3397 - skipped

SEQ ID NO: 3398 - protein, 452 aa, synthetic construct
QVQLQQSGTELMKPGASVKISCKASGYTFSNYWIEWIKQRPGHGLEWVGEILPGAGPTNYNEKFKGKATFTADSSSNTAYMQLSSLTSEDSAVYYCARTDYDYDWFAYWGQGTLVTVSAAKTTAPSVYPLAPVSGDTTGSSVTLGCLVKGYFPEPVTLTWNSGSLSSGVHTFPAVLQSDLYTLSSSVTVTSSTWPSQSITCNVAHPASSTKVDKKIEPKSCDKTHTCPPCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVCVLPPPEEEMTKKQVTLSCAVTDFMPEDIYVEWTNNGKTELNYKNTEPVLDSDGSYFMVSKLRVEKKNWVERNSYSCSVVHEGLHNHHTTKSFSRTPGKEPEA

SEQ ID NO: 3399 - protein, 220 aa, synthetic construct
DIVMSQSPSSLAVSVGEKVTMSCKSSQSLLYSGNQKNYLAWYQQKPGQSPKLLIYWASTRESGVPDRFTGSGSGTDFTLTINSVKAEDLTVYYCQQYYGYPRTFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3400 - protein, 383 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSDKTHTCPPCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPCEEEMTKKQVTLWCMVTDFMPEDIYVEWTNNGKTELNYKNTEPVLDSDGSYFMYSKLRVEKKNWVERNSYSCSVVHEGLHNHHTTKSFSRTPGKGGGGSHHHHHHHH

SEQ ID NO: 3401 - protein, 10 aa, synthetic construct
GYTFSNYWIE

SEQ ID NO: 3402 - protein, 17 aa, synthetic construct
EILPGAGPTNYNEKFKG

SEQ ID NO: 3403 - protein, 10 aa, synthetic construct
TDYDYDWFAY

SEQ ID NO: 3404 - protein, 17 aa, synthetic construct
KSSQSLLYSGNQKNYLA

SEQ ID NO: 3405 - protein, 7 aa, synthetic construct
WASTRES

SEQ ID NO: 3406 - protein, 9 aa, synthetic construct
QQYYGYPRT

SEQ ID NO: 3407 - skipped

SEQ ID NO: 3408 - protein, 446 aa, synthetic construct
QIQLVQSGPELKKPGETVKISCKASGYTFTDYSMHWMKQAPGKGLKWMAWINTDTGEPTYADDFKGRFAFSLETSVSTAYLQINNLKNEDTATYFCARSSRRFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 3409 - protein, 214 aa, synthetic construct
NIVMTQSPKSMSMSVGERVTLSCKASENVGTYVSWYQQKPEQSPKLLIYGASNRYTGVPDRFTGSGSAKDFTLTIRSVQAEDLADYHCGQSYISPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3410 - skipped

SEQ ID NO: 3411 - protein, 10 aa, synthetic construct
GYTFTDYSMH

SEQ ID NO: 3412 - protein, 17 aa, synthetic construct
WINTDTGEPTYADDFKG

SEQ ID NO: 3413 - protein, 7 aa, synthetic construct
SSRRFDY

SEQ ID NO: 3414 - protein, 11 aa, synthetic construct
KASENVGTYVS

SEQ ID NO: 3415 - protein, 7 aa, synthetic construct
GASNRYT

SEQ ID NO: 3416 - protein, 9 aa, synthetic construct
GQSYISPYT

SEQ ID NO: 3417-3419 - skipped (3 entries)

SEQ ID NO: 3420 - protein, 449 aa, synthetic construct
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3421 - protein, 5 aa, synthetic construct
LTYIH

SEQ ID NO: 3422-3427 - skipped (6 entries)

SEQ ID NO: 3428 - protein, 214 aa, synthetic construct
DIQMTQSPSFLSASVGDRVTITCKASQNVADRVVWHQQKPGKAPKALIYAASYLQSGVPSRFSGSGSGTEFTLTISSLQPEDFATYFCQQFKSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3429-3436 - skipped (8 entries)

SEQ ID NO: 3437 - protein, 214 aa, synthetic construct
DIQMTQSPSFLSASVGDRVTITCKASQNVADRVVWHQQKPGKAPKALIFAASYLQSGVPSRFSGSGSGTEFTLTISSLQPEDFATYFCQQFKSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3438 - protein, 117 aa, synthetic construct
EVQLVESGGGLVQPGGSLRLSCAASGFTFSNFGMHWVRQAPGKGLEWVSYISSGSSTIYYADTLKGRFTISRDNAKNSLYLQMNSLRAEDTAVYYCARRGEGAMDYWGQGTTVTVSS

SEQ ID NO: 3439-3442 - skipped (4 entries)

SEQ ID NO: 3443 - protein, 7 aa, synthetic construct
AASYLQS

SEQ ID NO: 3444 - skipped

SEQ ID NO: 3445 - protein, 118 aa, synthetic construct
QVQLVQSGAEVKKPGASVKVSCKASGYTFTDYNIHWVRQAPGQGLEWMGYINPYNGRTGYNQKFKARVTMTVDKSTSTAYMELSSLRSEDTAVYYCARWDGSSYFDYWGQGTLVTVSS

SEQ ID NO: 3446 - protein, 106 aa, synthetic construct
EIVLTQSPATLSVSPGERATLSCSASSSVSYMHWYQQKPGQAPRPLIYEISKLASGIPARFSGSGSGTEYTLTISSLQSEDFAVYYCQQWNYPLLTFGQGTKLEIK

SEQ ID NO: 3447 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 3448 - protein, 119 aa, synthetic construct
QVQLVQSGAEVKKPGASVKVSCKASGFNIKSAYMHWVRQAPGQEPGCMGRIDPATGKTKYAPKFQARVTMTADTSINTAYTELSSLRSEDTATYYCARSLNWDYGLDYWGQGTLVTVSS

SEQ ID NO: 3449 - protein, 111 aa, synthetic construct
EIVLTQSPATLSLSPGERATLSCRASKSVSILGTHLIHWYQQKPGLAPRLLIYAASNLESGIPDRFSGSGSETDFTLTISRLEPEDFAVYFCQQSIEDPWTFGGGTKVEIK

SEQ ID NO: 3450 - protein, 119 aa, synthetic construct
QVQLVQSGAEVKKPGASVKVSCKASGFNIKSAYMHWVRQAPGQGLEWMGRIDPATGKTKYAPKFQARVTMTADTSTNTAYMELSSLRSEDTAVYYCARSLNWDYGLDYWGQGTLVTVSS

SEQ ID NO: 3451 - protein, 111 aa, synthetic construct
EIVLTQSPGTLSLSPGERATLSCRASKSVSILGTHLIHWYQQKPGQAPRLLIYAASNLESGIPDRFSGSGSETDFTLTISRLEPEDFAVYFCQQSIEDPWTFGGGTKVEIK

SEQ ID NO: 3452 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 3453 - protein, 225 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 3454 - protein, 225 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 3455 - protein, 120 aa, synthetic construct
NVQLQESGPGLVKPSQSLSLTCSVAGYSITSGYFWNWIRQFPGNKLEWMGYISYDGSNNYNPSLKNRISITRDTSKNQFFLKLNSVTTEDTATYYCASPSPGTGYAVDYWGQGTSVTVSS

SEQ ID NO: 3456 - protein, 112 aa, synthetic construct
NVVMTQTPLSLPVSLGDQASISCRSSQSLVHSNGNTYLHWYLQKPGQSPKFLIYKVSNRFSGVPDRFSGGGSGTEFTLKISRVEAEDLGVYFCSQSTHVPFTFGSGTKLEIK

SEQ ID NO: 3457 - protein, 121 aa, synthetic construct
QVQLQQPGAELVRPGASVKLSCKASGYTFTNYWMNWVKQRPGQGLEWIGMIHPSDSATRLNQKFKDKATLTVDKSSSTAYMQLSSPTSEDSAVYYCARWAYYGNLYAIDYWGQGTSVTVSS

SEQ ID NO: 3458 - protein, 111 aa, synthetic construct
DIVLTQSPASLAVSLGQRATISCKASQSVDYDGYSYMHWFQQKPGQPPKLLIYAASNLESGIPARFIGSGSGTDFTLNIHPVEEEDAATYYCQQSNEDPWTFGGGTKLEIK

SEQ ID NO: 3459 - protein, 330 aa, synthetic construct
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVCVLPPPEEEMTKKQVTLSCAVTDFMPEDIYVEWTNNGKTELNYKNTEPVLDSDGSYFMVSKLRVEKKNWVERNSYSCSVVHEGLHNHHTTKSFSRTPGK

SEQ ID NO: 3460 - protein, 228 aa, synthetic construct
TIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVCVLPPPEEEMTKKQVTLSCAVTDFMPEDIYVEWTNNGKTELNYKNTEPVLDSDGSYFMVSKLRVEKKNWVERNSYSCSVVHEGLHNHHTTKSFSRTPGK

SEQ ID NO: 3461 - protein, 107 aa, synthetic construct
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3462 - protein, 228 aa, synthetic construct
TIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPCEEEMTKKQVTLWCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 3463 - protein, 121 aa, synthetic construct
EVQLVESGGGLVQPGASLKLSCVASGFTFSDYWMSWVRQTPGKTMEWIGNIKYDGSHTNYAPSLKNRFTISRDNAKSTLFLQMSNVRSEDTATYYCTRESHYGYIYPFDYWGQGVMVTVSS

SEQ ID NO: 3464 - protein, 107 aa, synthetic construct
DIQMTQSPSSMSVSLGDTVTITCRASQDVGIYVNWFQQKPGKSPRRMIYRATNLADGVPSRFSGSRSGSDYSLTISSLESEDVADYHCLQYDESPFTFGSGTKLEIK

SEQ ID NO: 3465 - protein, 119 aa, synthetic construct
QVQLQQSGTELMKPGASVKISCKASGYTFSNYWIEWIKQRPGHGLEWVGEILPGAGPTNYNEKFKGKATFTADSSSNTAYMQLSSLTSEDSAVYYCARTDYDYDWFAYWGQGTLVTVSA

SEQ ID NO: 3466 - protein, 113 aa, synthetic construct
DIVMSQSPSSLAVSVGEKVTMSCKSSQSLLYSGNQKNYLAWYQQKPGQSPKLLIYWASTRESGVPDRFTGSGSGTDFTLTINSVKAEDLTVYYCQQYYGYPRTFGGGTKVEIK

SEQ ID NO: 3467 - protein, 333 aa, synthetic construct
AKTTAPSVYPLAPVSGDTTGSSVTLGCLVKGYFPEPVTLTWNSGSLSSGVHTFPAVLQSDLYTLSSSVTVTSSTWPSQSITCNVAHPASSTKVDKKIEPKSCDKTHTCPPCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVCVLPPPEEEMTKKQVTLSCAVTDFMPEDIYVEWTNNGKTELNYKNTEPVLDSDGSYFMVSKLRVEKKNWVERNSYSCSVVHEGLHNHHTTKSFSRTPGKEPEA

SEQ ID NO: 3468 - protein, 231 aa, synthetic construct
DKTHTCPPCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVCVLPPPEEEMTKKQVTLSCAVTDFMPEDIYVEWTNNGKTELNYKNTEPVLDSDGSYFMVSKLRVEKKNWVERNSYSCSVVHEGLHNHHTTKSFSRTPGKEPEA

SEQ ID NO: 3469 - protein, 240 aa, synthetic construct
DKTHTCPPCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPCEEEMTKKQVTLWCMVTDFMPEDIYVEWTNNGKTELNYKNTEPVLDSDGSYFMYSKLRVEKKNWVERNSYSCSVVHEGLHNHHTTKSFSRTPGKGGGGSHHHHHHHH

SEQ ID NO: 3470 - protein, 116 aa, synthetic construct
QIQLVQSGPELKKPGETVKISCKASGYTFTDYSMHWMKQAPGKGLKWMAWINTDTGEPTYADDFKGRFAFSLETSVSTAYLQINNLKNEDTATYFCARSSRRFDYWGQGTTLTVSS

SEQ ID NO: 3471 - protein, 107 aa, synthetic construct
NIVMTQSPKSMSMSVGERVTLSCKASENVGTYVSWYQQKPEQSPKLLIYGASNRYTGVPDRFTGSGSAKDFTLTIRSVQAEDLADYHCGQSYISPYTFGGGTKLEIK

SEQ ID NO: 3472 - protein, 78 aa, synthetic construct
ITCPPPMSVEHADIWVKSYSLYSRERYICNSGFKRKAGTSSLTECVLNKATNVAHWTTPSLKCIRDPALVHQRPAPPS

SEQ ID NO: 3473 - protein, 20 aa, synthetic construct
GGSGGGGSGGGSGGGGSGGG

SEQ ID NO: 3474 - protein, 212 aa, synthetic construct
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3475 - protein, 107 aa, synthetic construct
DIQMTQSPSFLSASVGDRVTITCKASQNVADRVVWHQQKPGKAPKALIYAASYLQSGVPSRFSGSGSGTEFTLTISSLQPEDFATYFCQQFKSYPLTFGQGTKLEIK

SEQ ID NO: 3476 - protein, 107 aa, synthetic construct
DIQMTQSPSFLSASVGDRVTITCKASQNVADRVVWHQQKPGKAPKALIFAASYLQSGVPSRFSGSGSGTEFTLTISSLQPEDFATYFCQQFKSYPLTFGQGTKLEIK

SEQ ID NO: 3477 - protein, 27 aa, synthetic construct
  VARIANT 7..26: note = EAAAK repeats may be deleted
AEAAAKEAAAKEAAAKEAAAKEAAAKA

SEQ ID NO: 3478-3499 - skipped (22 entries)

SEQ ID NO: 3500 - protein, 370 aa, synthetic construct; identical to SEQ ID NO: 3340

SEQ ID NO: 3501 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3502 - protein, 449 aa, synthetic construct
QVQLVQSGAEVKKPGSSVKVSCKASGHDFRLTYIHWVRQAPGQGLEWMGRVSAGSGNVKYNEKFKGRVTITADTSTSTAYMELSSLRSEDTAVYYCAVSYYSYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 3503 - protein, 370 aa, synthetic construct; identical to SEQ ID NO: 3340

SEQ ID NO: 3504 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 3505 - protein, 227 aa, synthetic construct; identical to SEQ ID NO: 3447

SEQ ID NO: 3506-3641 - skipped (136 entries)

SEQ ID NO: 3642 - protein, 243 aa, synthetic construct
EVQLVESGGGLVQPGGSLRLSCAASGFTFSNFGMHWVRQAPGKGLEWVSYISSGSSTIYYADTLKGRFTISRDNSKNTLYLQMNSLRAEDTAVYYCARRGEGAMDYWGQGTTVTVSSGGGGSGGGGSGGGGSGGGGSDNQLTQSPSFLSASVGDRVTITCRASSSVNYIYWYQQKPGKAPKLLIYYTSNLAPGVPSRFSGSGSGNEYTLTISSLQPEDFATYYCQQFTSSPFTFGQGTKLEIK

SEQ ID NO: 3643 - protein, 16 aa, synthetic construct
GGGGSGGGGSGGGGGS

SEQ ID NO: 3644 - protein, 107 aa, synthetic construct [sequence omitted]

SEQ ID NO: 3645 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3646 - protein, 334 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKEPEA

SEQ ID NO: 3647 - protein, 330 aa, synthetic construct; identical to SEQ ID NO: 41

SEQ ID NO: 3648 - protein, 227 aa, synthetic construct; identical to SEQ ID NO: 3501

SEQ ID NO: 3649 - protein, 330 aa, synthetic construct; identical to SEQ ID NO: 3504

SEQ ID NO: 3650 - protein, 10 aa, synthetic construct
GHDFRLTYIH

SEQ ID NO: 3651 - protein, 17 aa, synthetic construct
RVSAGSGNVKYNEKFKG

SEQ ID NO: 3652 - protein, 11 aa, synthetic construct
KASQNVGNRVV

SEQ ID NO: 3653 - protein, 7 aa, synthetic construct
SSSHRYK

SEQ ID NO: 3654 - protein, 17 aa, synthetic construct
RIYAGSGNVKYNEKFKG

SEQ ID NO: 3655 - protein, 11 aa, synthetic construct
KASQNVADRVV

SEQ ID NO: 3656 - protein, 10 aa, synthetic construct
GHDFKLTYIH

SEQ ID NO: 3657 - protein, 17 aa, synthetic construct
RVSAGSGNTKYNEKFKG

SEQ ID NO: 3658 - protein, 11 aa, synthetic construct
KASQNVGDRVA

SEQ ID NO: 3659 - protein, 17 aa, synthetic construct
RVYAGSGNTKYNEKFKG

SEQ ID NO: 3660 - protein, 11 aa, synthetic construct
KASQNVEDRVV

SEQ ID NO: 3661 - protein, 17 aa, synthetic construct
RISAGSGNTKYNEKFKG

SEQ ID NO: 3662 - protein, 11 aa, synthetic construct
KASQNVGDRVV

SEQ ID NO: 3663 - protein, 17 aa, synthetic construct
RIYAGSGNTKYNEKFKG

SEQ ID NO: 3664 - protein, 9 aa, synthetic construct
LQHNSYPLT

SEQ ID NO: 3665 - protein, 17 aa, synthetic construct
RVSAGSGNVNYAQKFQG

SEQ ID NO: 3666 - protein, 11 aa, synthetic construct
RASQNVDNRLG

SEQ ID NO: 3667 - protein, 11 aa, synthetic construct
KASQNVDNRVA

SEQ ID NO: 3668 - protein, 7 aa, synthetic construct
AASSLQK

SEQ ID NO: 3669-3701 - skipped (33 entries)

SEQ ID NO: 3702 - protein, 7 aa, synthetic construct
FRLTYIH